(19) United States
(12) Patent Application Publication
(10) Pub. No.: US 2024/0398963 A1
(43) Pub. Date: Dec. 5, 2024

(54) DRUG DELIVERY SYSTEM FOR LOCALLY DELIVERING THERAPEUTIC AGENTS AND USES THEREOF

(71) Applicant: COVAL BIOPHARMA (SHANGHAI) CO., LTD., Shanghai (CN)

(72) Inventors: Guolong WU, Shanghai (CN); Weijiang ZHANG, Redwood City, CA (US); Lichun FENG, Shanghai (CN); Hao ZHANG, Foster City, CA (US); Dafeng LI, Shanghai (CN)

(21) Appl. No.: 18/692,340

(22) PCT Filed: Sep. 14, 2022
(86) PCT No.: PCT/CN2022/118658

§ 371 Date: Mar. 15, 2024

(30) Foreign Application Priority Data
Sep. 16, 2021 (WO) PCT/CN2021/118666

(51) Int. Cl. A61K 47/61; A61K 47/54; A61P 19/02

(52) U.S. Cl. CPC A61K 47/61; A61K 47/542; A61P 19/02

(57) ABSTRACT

Disclosed herein are drug delivery systems and methods for locally delivering therapeutic agents, and methods for using such drug delivery systems for the treatment of diseases.

### FIELD OF THE DISCLOSURE

The present disclosure relates to drug delivery systems and methods for locally delivering therapeutic agents, and methods for using such drug delivery systems for the treatment of diseases.

### BACKGROUND OF THE DISCLOSURE

Most of the therapeutic agents are delivered to the body systemically via oral/GI absorption or systemic injection. These delivery routes are convenient and suitable for treating systemic illnesses. However, more diseases are local disorders. Even though the theraputica agents administered systemically can effectively treat these disorders, they may also target other tissues or binding sites that can result in side effects or adverse effects. To reduce systemic side effects, a locally administered drug delivery system is desirable. Delivering therapeutic agents to the desirable sites is not as easy as taking drugs orally or via injections. Therefore, a long term, sustained release drug delivery system for locally delivering drug is a must for such a product to be acceptable by the doctors and patients. In addition, the release profile of the therapeutic agents to maintain an effective concentration at the delivery site after the drug being administered to a subject may dramatically affect the therapeutic agents' effectiveness. Thus, drug delivery of a therapeutic agent at a specific target tissue or site within the body represents a long-time challenge in the pharmaceutic industry.

Numerous drug delivery systems have been developed to provide controlled drug delivery with tissue specificity or desired release profile. The most common local drug delivery system is to use biodegradable polymers to control the release rate of the therapeutic agents. These drug delivery systems release drugs via both polymer erosion and drug molecule diffusion. This complicated release control has imposed a great challenge in drug product manufacturing and quality control.

There are three critical properties for a successful local drug delivery system: the ability to maintain the delivery system at the delivery site; the ability to release the therapeutic agent at a desirable rate and profile; and the ability to treat local disorder with the therapeutic agent. The present disclosure provides a different approach to fulfill these critical properties for a local drug delivery system, i.e., the biopolymers, due to their large molecular sizes, are to hold the drug delivery system at the delivery site; the therapeutic agents, are to be selected from marketed products or the activities have been proven by late stage clinical studies; and the linkers, covalently binding to the biopolymers and the therapeutic agents, are not stable chemically and upon degrading, release the therapeutic agents at a desirable rate for a specific delivery site and a specific disease.

### SUMMARY OF THE DISCLOSURE

In one aspect, the present disclosure provides a drug delivery system for locally delivering a therapeutic agent at a controlled rate, the drug delivery system comprising:
a biopolymer comprising at least a first binding group BG1 selected from the group consisting of carboxylic group, amino group and a combination thereof;
a therapeutic agent comprising at least a second binding group BG2 selected from the group consisting of hydroxyl group, carboxylic group, amino group, amide group, amine group and a combination thereof, and
a linker covalently linking the biopolymer to the therapeutic agent and capable of retaining the therapeutic agent in the location of administration;
wherein the linker comprises a structure of formula (I):

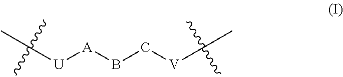

wherein
U is connected to the biopolymer through BG1 such that at least one amide linkage is formed, and U is selected from —N(R¹)— or

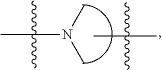

wherein

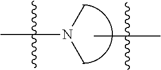

is a nitrogen-containing heteroaryl or a nitrogen-containing heterocyclyl optionally comprising one or more additional heteroatoms selected from N, O or S;
A is selected from a direct bond, alkyl and —(CH₂CH₂O)ₘ—, wherein said alkyl is optionally substituted with one or more R² groups;
B is selected from the group consisting of a direct bond, cycloalkyl, heterocyclyl, aryl, and heteroaryl, wherein each of cycloalkyl, heterocyclyl, aryl and heteroaryl is optionally substituted with one or more R³ groups;
C is selected from a direct bond, alkyl, or —[C(═O)NHCH₂]ₙ—, wherein said alkyl is optionally substituted with one or more R⁴ groups;
V is connected to the therapeutic agent through BG2 such that at least one linkage selected from the group consisting of a direct bond, ester, carbonate, carbamate, —C(═O)NHCH₂O— or —C(═O)OCH₂O— is formed, and V is selected from the group consisting of —C(═O)—, —OC(═O)—, —NHC(═O)—, —C(═O)NHCH₂— and —C(═O)OCH₂—;
R¹ is selected from the group consisting of hydrogen, alkyl, alkenyl and alkynyl;
each of R², R³, and R⁴ is independently selected from the group consisting of halogen, hydroxyl, amino, cyano, alkyl, alkoxyl, and —C(═O)OR⁵;
R⁵ is an alkyl;
m is an integer from 0 to 5; and
n is an integer from 1 to 4,
   provided that when the biopolymer is chondroitin sulfate, V is not —C(═O)—.

In some embodiments, the linker in the drug delivery system provided herein comprises a structure of formula (Ia) to (Ie):

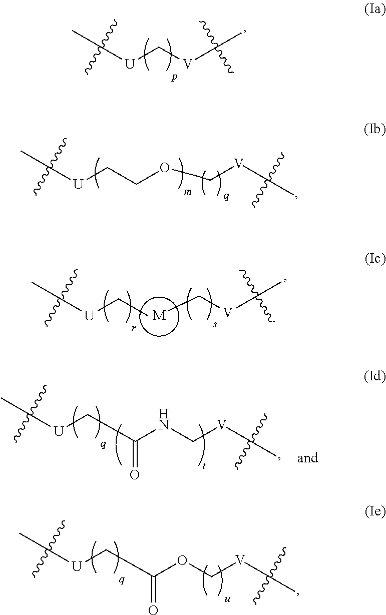

wherein,
U and V are as defined as supra;
M is selected from the group consisting of cycloalkyl, heterocyclyl, aryl, and heteroaryl, each of which is optionally substituted with one or more R³ groups;
each of

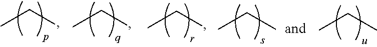

is optionally substituted with one or more group independently selected from alkyl or —C(═O)OCH₃;
p is an integer ranging from 0 to 10;
m and t are independently an integer ranging from 1 to 5;
q, r and s are independently an integer ranging from 0 to 5; and
t is an integer ranging from 1 to 5.

In a further aspect, the present disclosure provides a pharmaceutical composition comprising the drug delivery system provided herein and a pharmaceutically acceptable excipient.

In another aspect, the present disclosure provides a method of treating a disorder in a subject in need thereof, comprising administering to the subject a therapeutic effective amount of the drug delivery system or the pharmaceutical composition provided herein.

### BRIEF DESCRIPTION OF THE FIGURES

FIG. 1 illustrates the plasma and synovium time-concentration data of triamcinolone acetonide after a single IA dose of Example 2 (HA MW 1000 KDa).

FIG. 2 illustrates the plasma and synovium time-concentration data of triamcinolone acetonide after a single IA dose of Example 2 (HA MW 2000 KDa).

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, 2^{nd} Edition, University Science Books, Sausalito, 2006; Smith and March March's Advanced Organic Chemistry, 6^{th} Edition, John Wiley & Sons, Inc., New York, 2007; Larock, Comprehensive Organic Transformations, 3^{rd} Edition, VCH Publishers, Inc., New York, 2018; Carruthers, Some Modern Methods of Organic Synthesis, 4^{th} Edition, Cambridge University Press, Cambridge, 2004; the entire contents of each of which are incorporated herein by reference.

At various places in the present disclosure, linking substituents are described. It is specifically intended that each linking substituent includes both the forward and backward forms of the linking substituent. For example, —NR(CR′R″)— includes both —NR(CR′R″)— and —(CR′R″)NR—. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms. In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl (allyl), butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkenyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “alkoxyl”, whether as part of another term or used independently, refers to an alkyl group, as previously defined, attached to the parent molecule through an oxygen atom. The term “Cᵢ₋ⱼ alkoxy” means that the alkyl moiety of the alkoxy group has i to j carbon atoms. In some embodiments, alkoxy groups contain 1 to 10 carbon atoms. In some embodiments, alkoxy groups contain 1 to 9 carbon atoms. In some embodiments, alkoxy groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₆ alkoxyl” include, but are not limited to, methoxy, ethoxy, propoxy (e.g. n-propoxy and isopropoxy), t-butoxy, neopentoxy, n-hexoxy, and the like.

As used herein, the term “amide” refers to —C(═O)NR′—, wherein R′ represents hydrogen, an N-protecting group, alkyl, alkenyl, alkynyl, alkoxy, cycloalkyl, heterocyclyl, aryl, heteroaryl and other suitable organic groups.

As used herein, the term “amine” refers to derivatives of ammonia, wherein one or more hydrogen atoms are replaced by a substituent, and can be represented by N(H)ₙ(R′)₃₋ₙ wherein n is 0, 1, or 2, and each R′ is independently hydroxyl, nitro, an N-protecting group, alkyl, alkenyl, alkynyl, alkoxy, cycloalkyl, heterocyclyl, aryl, heteroaryl and other suitable organic groups, or two R′ together with the nitrogen atom to which they are attached form an optionally substituted heterocyclyl or heteroaryl.

As used herein, the term “amino” refers to —NH₂.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthyl, and the like. Aryl groups can be substituted at one or more ring positions with substituents as described above.

As used herein, the term “carbamate” refers to —N(R′)C(═O)O—, wherein R′ represents hydrogen, an N-protecting group, alkyl, alkenyl, alkynyl, alkoxy, cycloalkyl, heterocyclyl, aryl, heteroaryl and other suitable organic groups.

As used herein, the term “carbonate” refers to —OC(═O)O—.

As used herein, the term “carboxylic group” or “carboxyl” refers to —COOH.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system. In some embodiments, the cycloalkyl group may be monocyclic or polycyclic. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Examples of polycyclic cycloalkyl group include, but are not limited to, adamantyl, norbornyl, fluorenyl, spiro-pentadienyl, spiro[3.6]-decanyl, bicyclo[1,1,1]pentenyl, bicyclo[2,2,1]heptenyl, and the like.

As used herein, the term “cyano” refers to —CN.

As used herein, the term “ester” refers to —C(═O)O—.

As used herein, the term “halogen” refers to an atom selected from fluorine (or fluoro), chlorine (or chloro), bromine (or bromo) and iodine (or iodo).

As used herein, the term “heteroatom” refers to nitrogen, oxygen, or sulfur, and includes any oxidized form of nitrogen or sulfur, and any quaternized form of a basic nitrogen (including N-oxides).

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. “Heterocyclyl” also includes radicals wherein the heterocyclyl radicals are fused with a saturated, partially unsaturated, or fully unsaturated (i.e., aromatic) carbocyclic or heterocyclic ring. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

In some embodiments, the term “3- to 12-membered heterocyclyl” refers to a 3- to 12-membered saturated or partially unsaturated monocyclic or polycyclic heterocyclic ring system having 1 to 3 heteroatoms independently selected from nitrogen, oxygen, or sulfur. The fused, spiro and bridged ring systems are also included within the scope of this definition. Examples of monocyclic heterocyclyl include, but are not limited to oxetanyl, 1,1-dioxothietanylpyrrolidyl, tetrahydrofuryl, tetrahydrothienyl, pyrrolyl, furanyl, thienyl, pyrazolyl, imidazolyl, triazolyl, oxazolyl, thiazolyl, piperidyl, piperazinyl, piperidinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like. Examples of fused heterocyclyl include, but are not limited to, phenyl fused ring or pyridinyl fused ring, such as quinolinyl, isoquinolinyl, tetrahydroquinolinyl, tetrahydroisoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, imidazo[1,2-a]pyridinyl, [1,2,4]triazolo[4,3-a]pyridinyl, [1,2,3]triazolo[4,3-a]pyridinyl groups, and the like. Examples of spiro heterocyclyl include, but are not limited to, spiropyranyl, spirooxazinyl, and the like. Examples of bridged heterocyclyl include, but are not limited to, morphanyl, hexamethylenetetraminyl, 3-aza-bicyclo[3.1.0]hexane, 8-aza-bicyclo[3.2.1]octane, 1-aza-bicyclo[2.2.2]octane, 1,4-diazabicyclo[2.2.2]octane (DABCO), and the like.

As used herein, the term “hydroxyl” refers to —OH.

As used herein, the term “binding group” or “BG” refers to a group at a particular position within a first entity (e.g., biopolymer, therapeutic agent as provided herein), which is capable of reacting with another group from a second entity (e.g., linker as provided herein) to form a linkage, thereby joining the two entities together to form one entity. For example, carboxyl groups included in one entity may react with amino groups included in another entity to form amide linkage that links the two entities together, wherein the carboxyl and amino groups can be regarded as binding groups.

As used herein, the term “linkage” or “linker” refers to bonds or chemical moiety formed from a chemical reaction between the functional groups of at least two entities to be linked, thereby forming one molecule or maintaining association of the entities in sufficiently close proximity. A linker can be integrated in the resulting linked molecule or structure, with or without its reacted functional groups. Such linkages may be covalent or non-covalent. Hydrolytically unstable or degradable linkages mean that the linkages are degradable in water or in aqueous solutions, including for example, body fluid such as blood. Enzymatically unstable or degradable linkages mean that the linkage can be degraded by one or more enzymes. Such degradable linkages include, but are not limited to ester linkages formed by the carboxylic acid in one entity with alcohol groups on a biologically active agent, wherein such ester groups generally hydrolyze under physiological conditions to release the biologically active agent. Other hydrolytically degradable linkages include but are not limited to carbonate linkages, imine linkages resulted from reaction of an amine and an aldehyde, phosphate ester linkages resulted from reaction of a phosphate group and an alcohol, hydrazone linkages resulted from reaction of a hydrazide and an aldehyde, acetal linkages resulted from reaction of an aldehyde and an alcohol, amide linkages resulted from reaction of an amine group and a carboxyl group.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “pharmaceutically acceptable” indicates that the substance or composition is compatible chemically and/or toxicologically, with the other ingredients comprising a formulation, and/or the subjects being treated therewith.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

As used herein, the terms “therapeutic agent”, “drug”, “biologically active molecule”, “biologically active agent”, “active agent” and the like refer to any substance which can affect any physical or biochemical properties of a biological organism, including but not limited to viruses, bacteria, fungi, plants, animals, and human. In particular, as used herein, therapeutic agents include any substance intended for diagnosis, cure, mitigation, treatment, or prevention of disease in humans or other animals, or to otherwise enhance physical or mental well-being of humans or animals.

Drug delivery of therapeutic agents to specific tissues or sites within a body presents a variety of challenges, particularly where local delivery of a high dose of a therapeutic agent having poor aqueous solubility to a specific tissue is desired, and where avoidance of high systemic concentration of the therapeutic agent leading to toxic side effects is desired.

Therefore, the present disclosure in one aspect provides a drug delivery system capable of locally delivering a therapeutic agent at a controlled rate. In some embodiments, the drug delivery system comprises a biopolymer, a therapeutic agent and a linker covalently linking the biopolymer to the therapeutic agent and capable of retaining the therapeutic agent in the location of administration.

### Biopolymer

Biopolymers are natural polymers produced by living organisms, and contain monomeric units that are covalently bonded to form larger structures. There are three main classes of biopolymers, classified according to the monomeric units used and the structure of the biopolymer formed: polynucleotides, polypeptides, and polysaccharides. More specifically, polynucleotides, such as RNA and DNA, are long polymers composed of 13 or more nucleotide monomers. Polypeptides or proteins, are short polymers of amino acids and some major examples include collagen, actin, and fibrin. Polysaccharides, are often linear bonded polymeric carbohydrate structures and some examples include cellulose and alginate. Other examples of biopolymers include rubber, suberin, melanin and lignin.

A variety of biopolymers are useful as polymeric delivery vehicles for delivering the therapeutic agents to target cells or tissues. Biopolymers suitable for a particular application are selected based on their ability to target particular tissues, organs or cells, and their in vivo stability, i.e., the in vivo residence time in the circulatory system, or specific tissues, cells or organs.

In some embodiments, the biopolymer is selected from biocompatible polymers comprising at least a first binding group BG1, which is capable of reacting with a reactive functional group from a second entity (e.g., linker as provided herein) to form a linkage, thereby linking a biopolymer to the second entity (e.g., the linker). The term “biocompatible”, as used herein, refers to a substance that has no medically unacceptable toxic or injurious effects on biological function, or which is tolerated by the body.

In some embodiments, the biopolymer is selected from biocompatible polymers comprising at least a first binding group BG1, wherein BG1 is selected from the group consisting of hydroxyl group, carboxylic group, amino group, and a combination thereof. The BG1 serves as binding sites for the conjugation of linkers suitable for linking the therapeutic agents to the biopolymer. The BG1 may be present at any site within the backbone of the biopolymer, and thus the linkages formed between the biopolymer and the linker may be present at any part of the biopolymer.

In some embodiments, the BG1 for reacting with the reactive functional group from the linker can be the same or different. In certain embodiments, the BG1 of the biopolymer is the same. In certain embodiments, the BG1 of the biopolymer is different.

In some embodiments, the biopolymers are biocompatible polymers comprising carboxylic group as BG1, which is capable of reacting with a reactive functional group of a suitable linker to form a linkage connecting the carboxylic group-containing biopolymer to the linker.

In certain embodiments, the reactive functional group of the linker is amino or amine, which reacts with the carboxylic group of the biopolymer such that an amide linkage is formed.

In some embodiments, the biopolymers are biocompatible polymers comprising amino or amine group as BG1, which is capable of reacting with a reactive functional group of a suitable linker to form a linkage, thereby producing a biopolymer-linker conjugate.

In certain embodiments, the reactive functional group of the linker is a carboxylic group, which reacts with the amino or amine group of the biopolymer such that an amide linkage is formed.

In some embodiments, the linkage formed from the reaction between the BG1 of the biopolymer and the reactive functional group of the linker is selected from —C(O)N(R′)— or

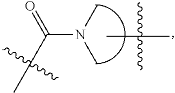

wherein R¹ is selected from the group consisting of hydrogen, alkyl, alkenyl

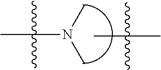

and alkynyl is a nitrogen-containing heteroaryl or a nitrogen-containing heterocyclyl optionally comprising one or more additional heteroatoms selected from N, O or S.

In certain embodiments, R¹ is hydrogen.

In certain embodiments, R¹ is alkyl. In certain embodiments, R¹ is C₁₋₆ alkyl, C₁₋₅ alkyl, C₁₋₄ alkyl, or C₁₋₃ alkyl. In certain embodiments, R¹ is methyl.

In certain embodiments, R¹ is alkenyl. In certain embodiments, R¹ is C₂₋₆ alkenyl, C₂₋₅ alkenyl, C₂₋₄ alkenyl, or C₂₋₃ alkenyl. In certain embodiments, R¹ is vinyl.

In certain embodiments, R¹ is alkynyl. In certain embodiments, R¹ is C₂₋₆ alkynyl, C₂₋₅ alkynyl, C₂₋₄ alkynyl, or C₂₋₃ alkynyl. In certain embodiments, R¹ is ethynyl.

In certain embodiments,

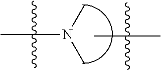

selected from the group consisting of:

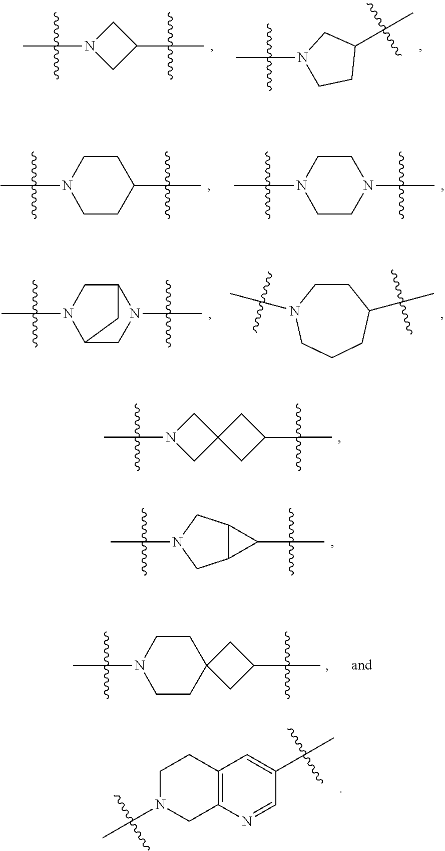

In some embodiments, the biopolymer may be selected from the group consisting of hyaluronic acid (HA), dextran, cellulose, amylose, chitosan, chitin, chondroitin, chondroitin sulfate (CS), gelatin, alginate, carrageenan, gellan, guar gum, pectin, scleroglucan, xanthan, or derivatives thereof.

In some embodiments, the biopolymer may have a number average molecular weight ranging from 400 to 3,000,000 Da, for example, from 1,000 to 3,000,000 Da, from 5,000 to 3,000,000 Da, from 10,000 to 3,000,000 Da, from 20,000 to 3,000,000 Da, from 30,000 to 3,000,000 Da, from 40,000 to 3,000,000 Da, from 50,000 to 3,000,000 Da, or from 50,000 to 2,000,000 Da.

In some embodiments, the biopolymer may be selected from the group consisting of HA, chitosan, chondroitin sulfate, or derivatives thereof.

In certain embodiments, the biopolymer is HA. In certain embodiments, the HA can derive from any source.

In certain embodiments, the biopolymer is CS. In certain embodiments, the HA can derive from any source.

In certain embodiments, the HA may have a number average molecular weight ranging from 400 to 3,000,000 Da, for example, from 1,000 to 3,000,000 Da, from 5,000 to 3,000,000 Da, from 10,000 to 3,000,000 Da, from 20,000 to 3,000,000 Da, from 30,000 to 3,000,000 Da, from 40,000 to 3,000,000 Da, from 50,000 to 3,000,000 Da, or from 50,000 to 2,000,000 Da.

In certain embodiments, the biopolymer is chondroitin sulfate. In certain embodiments, the chondroitin sulfate can derive from any source.

In certain embodiments, the chondroitin sulfate may have a number average molecular weight ranging from 400 to 3,000,000 Da, for example, from 1,000 to 3,000,000 Da, from 5,000 to 3,000,000 Da, from 10,000 to 3,000,000 Da, from 20,000 to 3,000,000 Da, from 30,000 to 3,000,000 Da, from 40,000 to 3,000,000 Da, from 50,000 to 3,000,000 Da, or from 50,000 to 2,000,000 Da.

### Therapeutic Agent

The present disclosure provides improved delivery system for local delivery of a variety of therapeutic agent.

In some embodiments, the therapeutic agent comprises at least a second binding group BG2, which is capable of reacting with a reactive functional group from a second entity (e.g., linker as provided herein) and an optional co-reactant to form a linkage, thereby linking the therapeutic agent to the second entity (e.g., the linker).

In some embodiments, the therapeutic agent comprises at least a second binding group BG2 selected from the group consisting of hydroxyl group, carboxylic group, amino group, amide group, amine group and a combination thereof. The BG2 serves as a binding site for the conjugation of linkers suitable for linking the therapeutic agents to the biopolymer.

In some embodiments, the therapeutic agent comprises hydroxyl group as BG2, which is capable of reacting with a reactive functional group of a suitable linker and an optional co-reactant to form a linkage connecting the hydroxyl-containing therapeutic agent to the linker.

In certain embodiments, the hydroxyl group in the therapeutic agent reacts with the reactive functional group of a linker and an optional co-reactant such that the therapeutic agent is linked to the linker via an ester linkage.

In certain embodiments, the hydroxyl group in the therapeutic agent reacts with the reactive functional group of a linker and an optional co-reactant such that the therapeutic agent is linked to the linker via a carbonate linkage.

In certain embodiments, the hydroxyl group in the therapeutic agent reacts with the reactive functional group of a linker and an optional co-reactant such that the therapeutic agent is linked to the linker via a carbamate linkage.

In certain embodiments, the hydroxyl group in the therapeutic agent reacts with the reactive functional group of a linker and an optional co-reactant such that the therapeutic agent is linked to the linker via a —C(═O)NHCH₂O— linkage.

In certain embodiments, the hydroxyl group in the therapeutic agent reacts with the reactive functional group of a linker and an optional co-reactant such that the therapeutic agent is linked to the linker via a —C(═O)OCH₂O— linkage.

In some embodiments, the therapeutic agent comprises carboxylic group as BG2, which is capable of reacting with a reactive functional group of a suitable linker and an optional co-reactant to form a linkage connecting the carboxylic group-containing therapeutic agent to the linker.

In certain embodiments, the carboxylic group in the therapeutic agent reacts with the reactive functional group of a linker and an optional co-reactant such that the therapeutic agent is linked to the linker via an ester linkage.

In some embodiments, the therapeutic agent comprises amino or amine group as BG2, which is capable of reacting with a reactive functional group of a suitable linker and an optional co-reactant to form a linkage connecting the amino/amine-containing therapeutic agent to the linker.

In certain embodiments, the amine group in the therapeutic agent reacts with the reactive functional group of a linker and an optional co-reactant such that the therapeutic agent is linked to the linker via a direct bond, an amide linkage, a urea linkage, a thiourea linkage, a carbamate linkage, a thiocarbamate linkage, an aza-acetal linkage, a phosphoramidate linkage, and the like.

In some embodiments, the therapeutic agent to be delivered is anti-inflammatory drugs,

In some embodiments, the therapeutic agent to be delivered is antiflammatory drugs selected from the group consisting of triamcinolone acetonide, meprednisone, prednisolone, hydrocortisone, cortisone, fluocinonide, methylprednisolone, betamethason, and dexamethasone.

### Linker

The improved local delivery of therapeutic agents is achieved by linking a therapeutic agent to a biopolymer via a suitable linker. By selecting suitable linkers, the releasing rate of the therapeutic agent from the biopolymer can be controlled, thereby providing improved delivery of the therapeutic agent to target cells or tissues.

In some embodiments, a plurality of the linkers can be attached to a therapeutic agent via a cleavable linkage which is cleaved under biological conditions, thereby releasing the therapeutic agent.

A “cleavable linkage” is a relatively labile bond that cleaves under physiological conditions. An exemplary releasable linkage is a hydrolyzable bond that cleaves upon reaction with water (i.e., is hydrolyzed). The tendency of a bond to hydrolyze in water may depend not only on the general type of linkage connecting two atoms but also on the substituents attached to these atoms. Appropriate hydrolytically unstable or weak linkages include but are not limited to carboxylate ester, phosphate ester, anhydrides, acetals, ketals, acyloxyalkyl ether, imines, orthoesters, peptides, oligonucleotides, thioesters, urea, thiourea, carbamate, thiocabamate, phosphoramidate and carbonates. Certain functional groups have atoms that may be chemically degraded by a process other than hydrolysis. Exemplary releaseable linkages in this category include certain carbamates and Fmoc derivatives. Certain molecules containing these kinds of functionalities appropriately bonded may undergo chemical degradation (release) upon action of a base. In such cases “cleave” may occur at higher values of pH or through the action of biological molecules that contain basic moieties (e.g. histidines). Another exemplary cleavable linkage is an enzymatically cleavable linkage. An “enzymatically cleavable linkage” means a linkage that is subject to cleavage by one or more enzymes.

In some embodiments, the linker is attached to the biopolymer via a linkage formed from a reactive functional group of the linker and the BG1 in the biopolymer, and is attached to the therapeutic agent via a linkage formed from another reactive functional group of the linker and the BG2 in the therapeutic agent.

In some embodiments, the linker comprises a structure of formula (I):

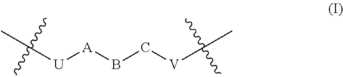

wherein
U is connected to the biopolymer through BG1 such that at least one amide linkage is formed, and U is selected from —N(R′)— or

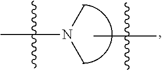

wherein

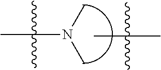

is a nitrogen-containing heteroaryl or a nitrogen-containing heterocyclyl optionally comprising one or more additional heteroatoms selected from N, O or S;
A is selected from a direct bond, alkyl and —(CH₂CH₂O)ₘ—, wherein said alkyl is optionally substituted with one or more R² groups;
B is selected from the group consisting of a direct bond, cycloalkyl, heterocyclyl, aryl, and heteroaryl, wherein each of cycloalkyl, heterocyclyl, aryl and heteroaryl is optionally substituted with one or more R³ groups;
C is selected from a direct bond, alkyl, or —[C(═O)NHCH₂]ₙ—, wherein said alkyl is optionally substituted with one or more R⁴ groups;
V is connected to the therapeutic agent through BG2 such that at least one linkage selected from the group consisting of a direct bond, ester, carbonate, carbamate, —C(═O)NHCH₂O— or —C(═O)OCH₂O— is formed, and V is selected from the group consisting of —C(═O)—, —OC(═O)—, —NHC(═O)—, —C(═O)NHCH₂— and —C(═O)OCH₂—;
R¹ is selected from the group consisting of hydrogen, alkyl, alkenyl and alkynyl;
each of R², R³, and R⁴ is independently selected from the group consisting of halogen, hydroxyl, amino, cyano, alkyl, alkoxyl, and —C(═O)OR⁵;
R⁵ is an alkyl;
m is an integer from 0 to 5; and
n is an integer from 1 to 4,
provided that when the biopolymer is chondroitin sulfate, V is not —C(═O)—.

In some embodiments, BG1 is a carboxylic group and U is —N(R¹)—, such that an amide linkage is formed to attach the biopolymer to the linker.

In some embodiments, BG1 is a carboxylic group and U is

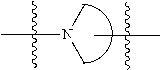

such that an amide linkage is formed to attach the biopolymer to the linker.

In certain embodiments, BG1 is a carboxylic group and U is

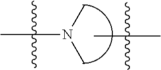

selected from the group consisting of:

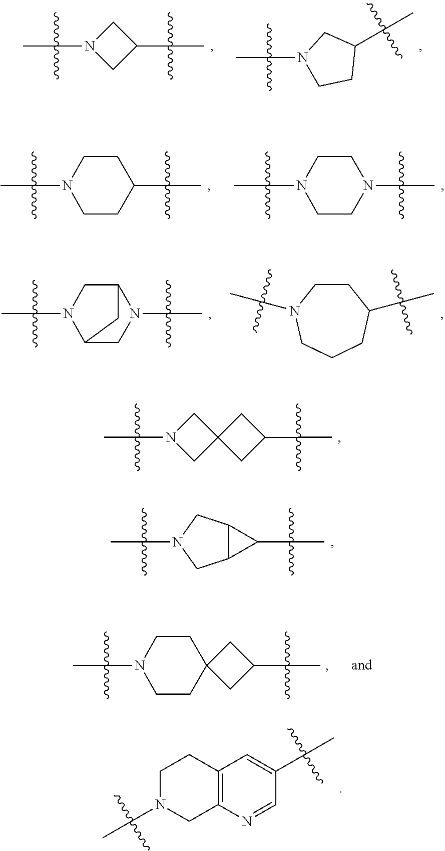

In some embodiments, BG2 is a hydroxyl group, and V is selected from one of the following:
(a) —C(═O)— which is connected to the therapeutic agent through BG2 such that an ester linkage is formed;
(b) —OC(═O)— which is connected to the therapeutic agent through BG2 such that a carbonate linkage is formed;
(c) —NHC(═O)— which is connected to the therapeutic agent through BG2 such that a carbamate linkage is formed;
(d) —C(═O)NHCH₂— which is connected to the therapeutic agent through BG2 such that —C(═O)NHCH₂O— linkage is formed; or
(e) —C(═O)OCH₂— which is connected to the therapeutic agent through BG2 such that —C(═O)OCH₂O— linkage is formed.

In some embodiments, A is a direct bond.

In some embodiments, A is alkyl optionally substituted with one or more R² groups. In certain embodiments, A is C₁₋₁₀ alkyl optionally substituted with one or more R² groups. In certain embodiments, A is C₁₋₉ alkyl optionally substituted with one or more R² groups. In certain embodiments, A is C₁₋₈ alkyl optionally substituted with one or more R² groups. In certain embodiments, A is C₁₋₇ alkyl optionally substituted with one or more R² groups.

In certain embodiments, each R² is independently selected from alkyl or —C(═O)OR⁵.

In certain embodiments, each R² is independently C₁₋₆ alkyl, C₁₋₅ alkyl, C₁₋₄ alkyl, or C₁₋₃ alkyl. In certain embodiments, R² is methyl.

In certain embodiments, each R² is independently —C(═O)OR⁵, wherein R⁵ is C₁₋₆ alkyl, C₁₋₅ alkyl, C₁₋₄ alkyl, or C₁₋₃ alkyl. In certain embodiments, R² is —C(═O)OCH₃.

In some embodiments, A is —(CH₂CH₂O)ₘ—.

In certain embodiments, m is an integer from 0 to 4. In certain embodiments, m is 1. In certain embodiments, m is 2. In certain embodiments, m is 3. In certain embodiments, m is 4.

In some embodiments, B is a direct bond.

In some embodiments, B is a cycloalkyl.

In certain embodiments, B is 3 to 8 membered cycloalkyl, 3 to 7 membered cycloalkyl, 3 to 6 membered cycloalkyl, 3 to 5 membered cycloalkyl, or 3 to 4 membered cycloalkyl.

In certain embodiments, B is selected from the group consisting of cyclobutyl, cyclopentyl, cyclohexyl, and bicycle[2.2.2]octyl.

In some embodiments, B is an aryl. In certain embodiments, B is 5 to 12 membered aryl, 5 to 10 membered aryl, 5 to 8 membered aryl, or 5 to 6 membered aryl.

In certain embodiments, B is a phenyl.

In some embodiments, B is a heteroaryl. In certain embodiments, B is 5 to 12 membered heteroaryl, 5 to 10 membered heteroaryl, 5 to 8 membered heteroaryl, or 5 to 6 membered heteroaryl.

In certain embodiments, B is a pyridinyl or furyl.

In some embodiments, A is a direct bond, and B is selected from the group consisting of a direct bond, cycloalkyl, and aryl.

In certain embodiments, A is a direct bond, and B is a direct bond.

In certain embodiments, A is a direct bond, and B is 3 to 8 membered cycloalkyl, 3 to 7 membered cycloalkyl, 3 to 6 membered cycloalkyl, 3 to 5 membered cycloalkyl, or 3 to 4 membered cycloalkyl. In certain embodiments, A is a direct bond, and B is a cyclobutyl, cyclohexyl or bicycle[2.2.2]octyl.

In certain embodiments, A is a direct bond, and B is 5 to 12 membered aryl, 5 to 10 membered aryl, 5 to 8 membered aryl, or 5 to 6 membered aryl. In certain embodiments, A is a direct bond, and B is a phenyl.

In certain embodiments, A is a direct bond, and B is 5 to 12 membered heteroaryl, 5 to 10 membered heteroaryl, 5 to 8 membered heteroaryl, or 5 to 6 membered heteroaryl. In certain embodiments, A is a direct bond, and B is a pyridyl or furyl.

In some embodiments, A is an alkyl optionally substituted with one or more R² groups, and B is selected from the group consisting of a direct bond, aryl, and heteroaryl.

In certain embodiments, A is C₁₋₈ alkyl optionally substituted with one or more R² groups, and B is a direct bond, wherein R² is alkyl or —C(═O)OR⁵, wherein R⁵ is an alkyl.

In certain embodiments, A is C₁₋₈ alkyl optionally substituted with one or more R² groups, and B is 5 to 12 membered aryl, 5 to 10 membered aryl, 5 to 8 membered aryl, or 5 to 6 membered aryl. In certain embodiments, A is C₁₋₈ alkyl optionally substituted with one or more R² groups, and B is phenyl.

In certain embodiments, A is C₁₋₈ alkyl optionally substituted with one or more R² groups, and B is a heteroaryl. In certain embodiments, A is C₁₋₈ alkyl optionally substituted with one or more R² groups, and B is a pyridinyl or furyl.

In some embodiments, A is —(CH₂CH₂O)ₘ—, wherein m is an integer from 0 to 4, and B is a direct bond.

In some embodiment, C is a direct bond.

In some embodiments, C is alkyl optionally substituted with one or more R⁴ groups. In certain embodiments, C is C₁₋₆ alkyl optionally substituted with one or more R⁴ groups. In certain embodiments, C is C₁₋₅ alkyl optionally substituted with one or more R⁴ groups. In certain embodiments, C is C₁₋₄ alkyl optionally substituted with one or more R⁴ groups. In certain embodiments, C is C₁₋₃ alkyl optionally substituted with one or more R⁴ groups. In certain embodiments, C is a methyl or ethyl.

In certain embodiments, each R⁴ is independently selected from alkyl or —C(═O)OR⁵.

In certain embodiments, each R⁴ is independently C₁₋₆ alkyl, C₁₋₅ alkyl, C₁₋₄ alkyl, or C₁₋₃ alkyl. In certain embodiments, R⁴ is a methyl.

In certain embodiments, each R⁴ is independently —C(═O)OR⁵, wherein R⁵ is C₁₋₆ alkyl, C₁₋₅ alkyl, C₁₋₄ alkyl, or C₁₋₃ alkyl. In certain embodiments, R⁴ is —C(═O)OCH₃.

In some embodiment, C is —[C(═O)NHCH₂]ⁿ—.

In some embodiments, A is a direct bond, B is a direct bond, cycloalkyl or aryl, and C is selected from the group consisting of a direct bond or alkyl optionally substituted with one or more R⁴ groups.

In certain embodiments, A is a direct bond, B is a direct bond, and C is a direct bond.

In certain embodiments, A is a direct bond, B is a cycloalkyl, and C is a direct bond.

In certain embodiments, A is a direct bond, B is an aryl, and C is a direct bond or alkyl optionally substituted with one or more R⁴ groups.

In some embodiments, A is an alkyl optionally substituted with one or more R² groups, B is selected from the group consisting of a direct bond, aryl, and heteroaryl, and C is selected from a direct bond, alkyl optionally substituted with one or more R⁴ groups, or —[C(═O)NHCH₂]ₙ—.

In certain embodiments, A is C₁₋₈ alkyl optionally substituted with one or more R² groups, B is a direct bond, and C is a direct bond or —[C(═O)NHCH₂]ₙ—.

In certain embodiments, A is C₁₋₈ alkyl optionally substituted with one or more R² groups, B is 5 to 12 membered aryl, 5 to 10 membered aryl, 5 to 8 membered aryl, or 5 to 6 membered aryl, and C is a direct bond. In certain embodiments, A is C₁₋₈ alkyl optionally substituted with one or more R² groups, B is phenyl, and C is a direct bond.

In certain embodiments, A is C₁₋₈ alkyl optionally substituted with one or more R² groups, B is heteroaryl, and C is a direct bond. In certain embodiments, A is C₁₋₈ alkyl optionally substituted with one or more R² groups, B is pyridinyl or furyl, and C is a direct bond.

In some embodiments, A is —(CH₂CH₂O)ₘ—, B is an alkyl, and C is a direct bond.

In some embodiments, A is —(CH₂CH₂O)ₘ—, wherein m is 1, 2, 3, or 4, B is C₁₋₆ alkyl, C₁₋₅ alkyl, C₁₋₄ alkyl, C₁₋₃ alkyl, or C₁₋₂ alkyl, and C is a direct bond.

In some embodiments, A is —(CH₂CH₂O)ₘ—, B is a direct bond, and C is a direct bond.

In some embodiments, the linker provided herein comprises a structure of formula (Ia) to (Ie):

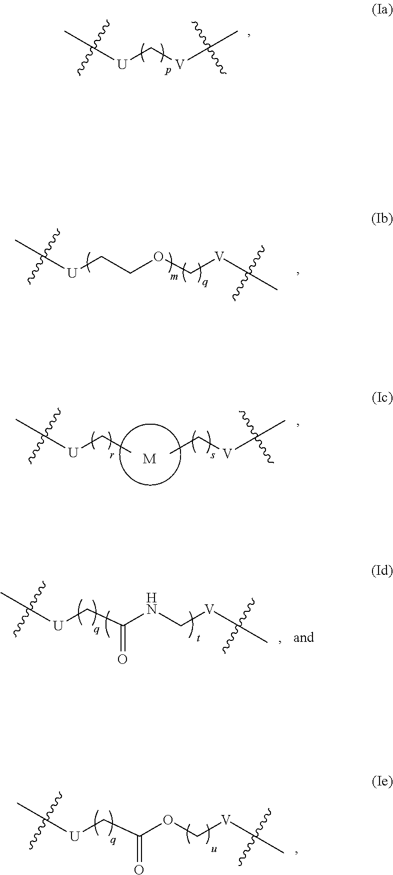

wherein,
U and V are defined as supra;
M is selected from the group consisting of cycloalkyl, heterocyclyl, aryl, and heteroaryl, each of which is optionally substituted with one or more R³ groups;
each of

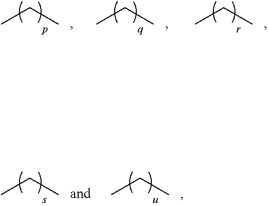

is optionally substituted with one or more group independently selected from alkyl or —C(═O)OCH₃;
p is an integer ranging from 0 to 10;
m and t are independently integers ranging from 1 to 5;
q, r and s are independently integers ranging from 0 to 5; and
t is an integer ranging from 1 to 5.

In some embodiments, M is selected from the group consisting of cyclobutyl, cyclopentyl, cyclohexyl, bicycle[2.2.2]octyl, phenyl, pyridyl and furyl.

In some embodiments, the linker provided herein comprises a structure selected from the group consisting of:

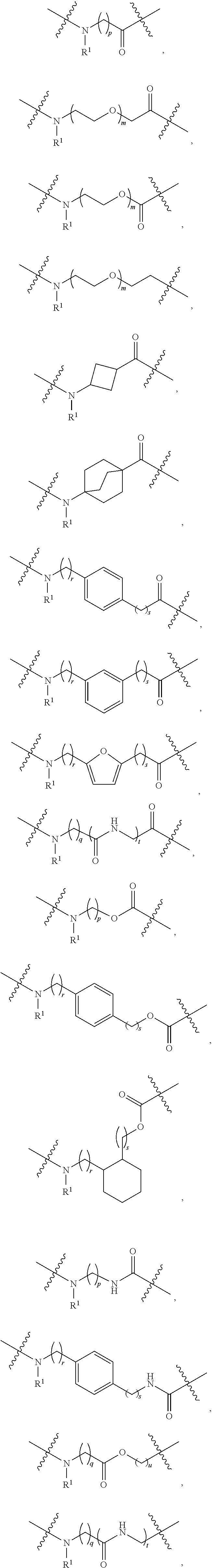

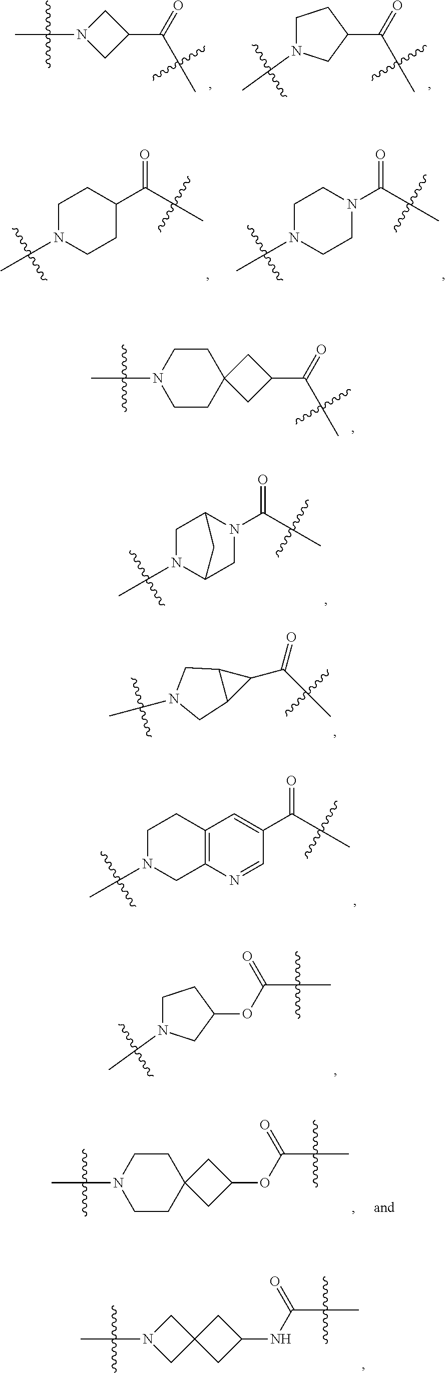

wherein each of

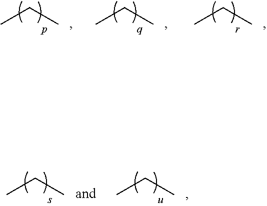

is optionally substituted with one or more group independently selected from alkyl or —C(═O)OCH₃.

### Drug Delivery System

In an aspect of the present disclosure, the therapeutic agent is attached to the biopolymer via a linker, thereby providing the drug delivery system for local delivery of the therapeutic agent to target sites.

The biopolymer of the drug delivery system provided herein may have one or more therapeutic agents conjugated via the linker. The biopolymer may be conjugated to the one or more therapeutic agents via one or more linkers at carboxylic group and/or amino group in the backbone of the biopolymer.

The drug delivery system of the present disclosure is obtained by conjugation between the biopolymer and the therapeutic agent by means of the linker through the formation of linkages between the biopolymer and the linker and linkage between the therapeutic agent and the linker.

In some embodiments, a reactive functional group of the linker, may first react with the BG2 of the therapeutic agent to form a linkage between the therapeutic agent and the linker, thereby providing a therapeutic agent-linker conjugate. The therapeutic agent-linker conjugate, which contains another reactive functional group at the terminal of the linker, may subsequently react with the BG1 of the biopolymer to form a linkage between the biopolymer and the linker, thereby providing the drug delivery system of the present disclosure.

In certain embodiments, it is possible to first react the BG1 of the biopolymer with a reactive functional group of the linker to form a biopolymer-linker conjugate, and subsequently react the BG2 of the therapeutic agent with another functional group of the linker in the biopolymer-linker conjugate, thereby providing the drug delivery system of the present disclosure.

In some embodiments, the biopolymer selected for the drug delivery system provided herein is hyaluronic acid or chondroitin sulfate, and the therapeutic agent selected for the drug delivery system provided herein is selected from the group consisting of triamcinolone acetonide, meprednisone, prednisolone, hydrocortisone, cortisone, fluocinonide, methylprednisolone, betamethason, and dexamethasone.

In certain embodiments, the biopolymer selected for the drug delivery system provided herein is hyaluronic acid, and the therapeutic agent selected for the drug delivery system provided herein is selected from the group consisting of triamcinolone acetonide, meprednisone, prednisolone, hydrocortisone, cortisone, fluocinonide, methylprednisolone, betamethason, and dexamethasone.

In certain embodiments, the drug delivery system provided herein is selected from the group consisting of:

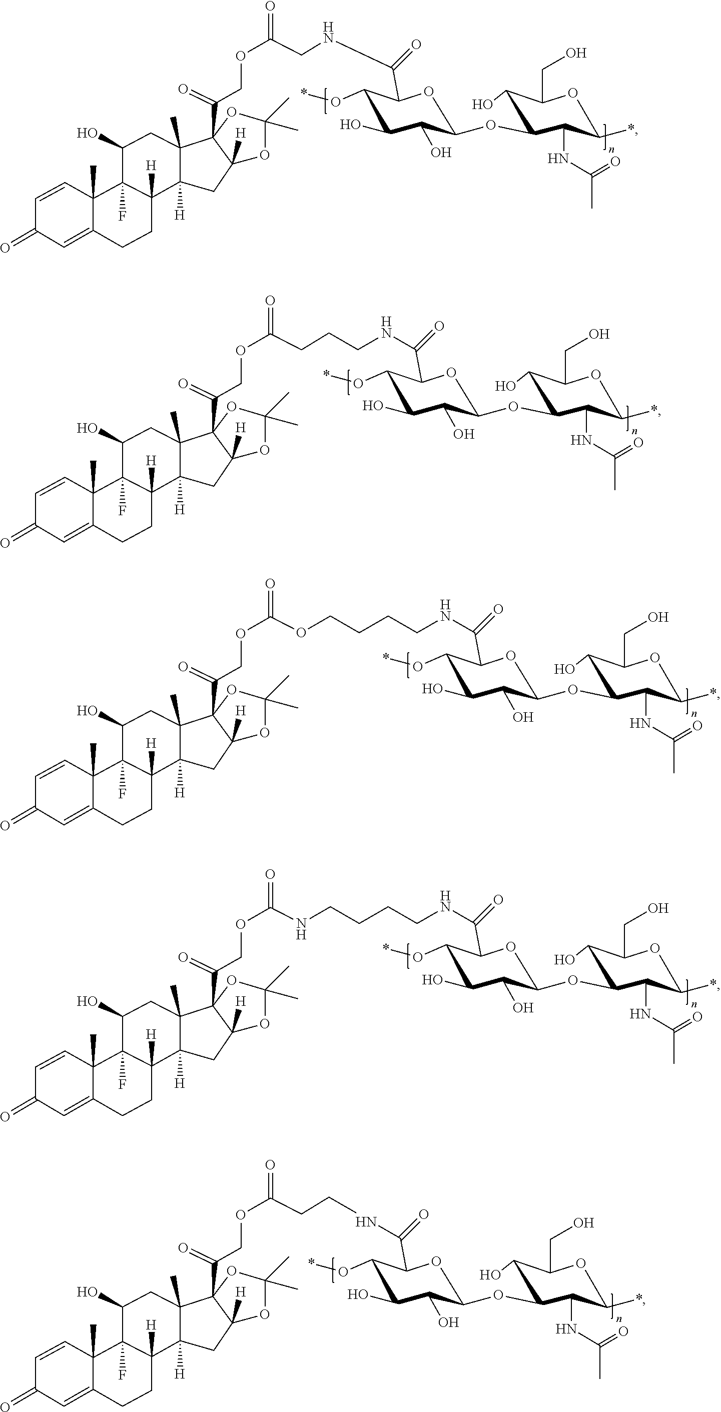

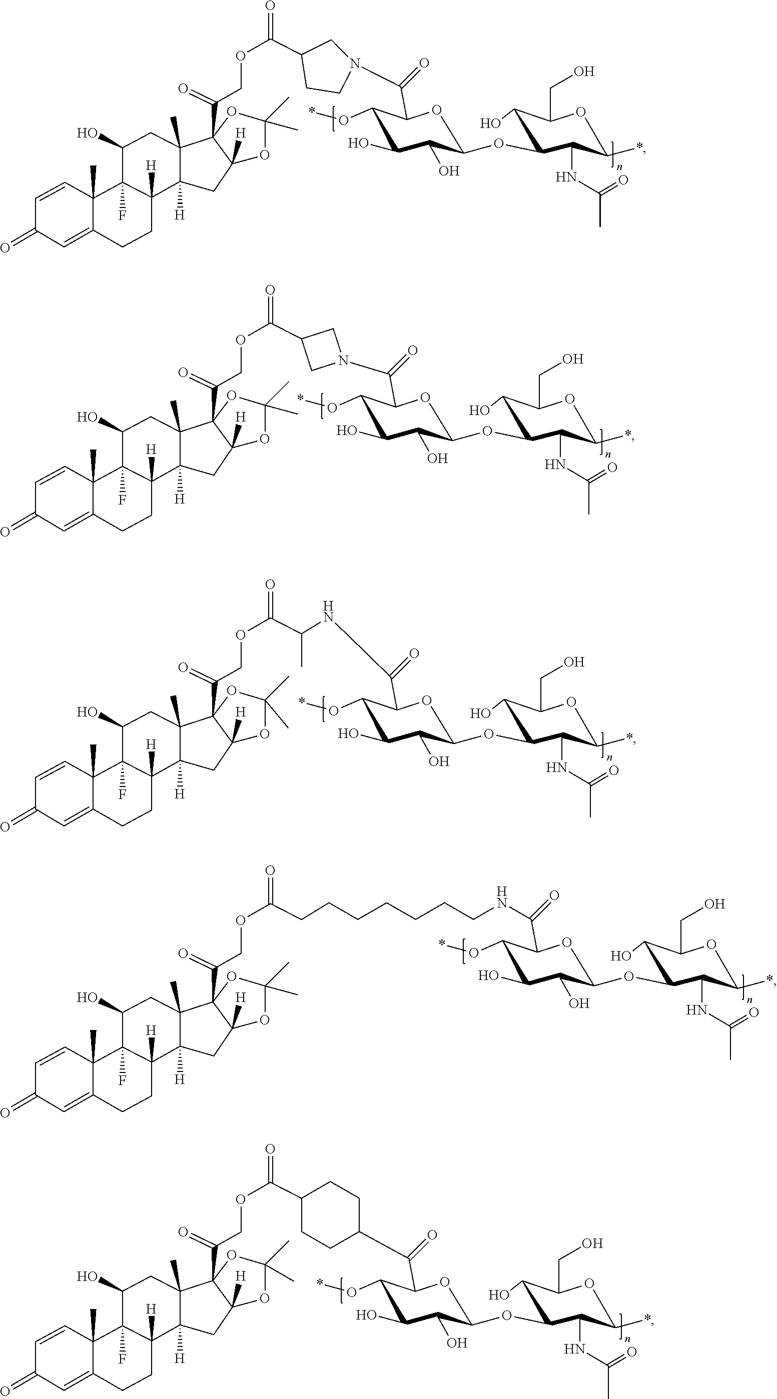

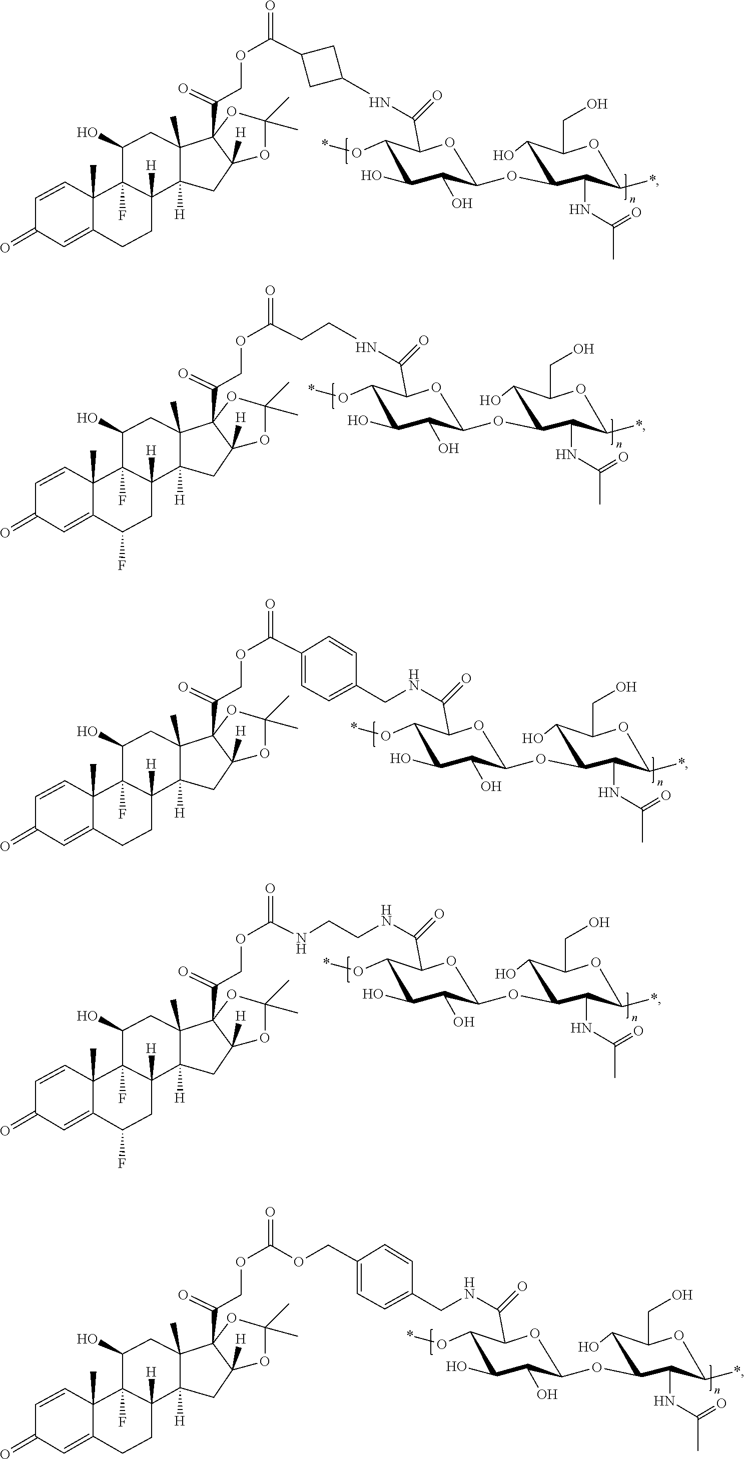

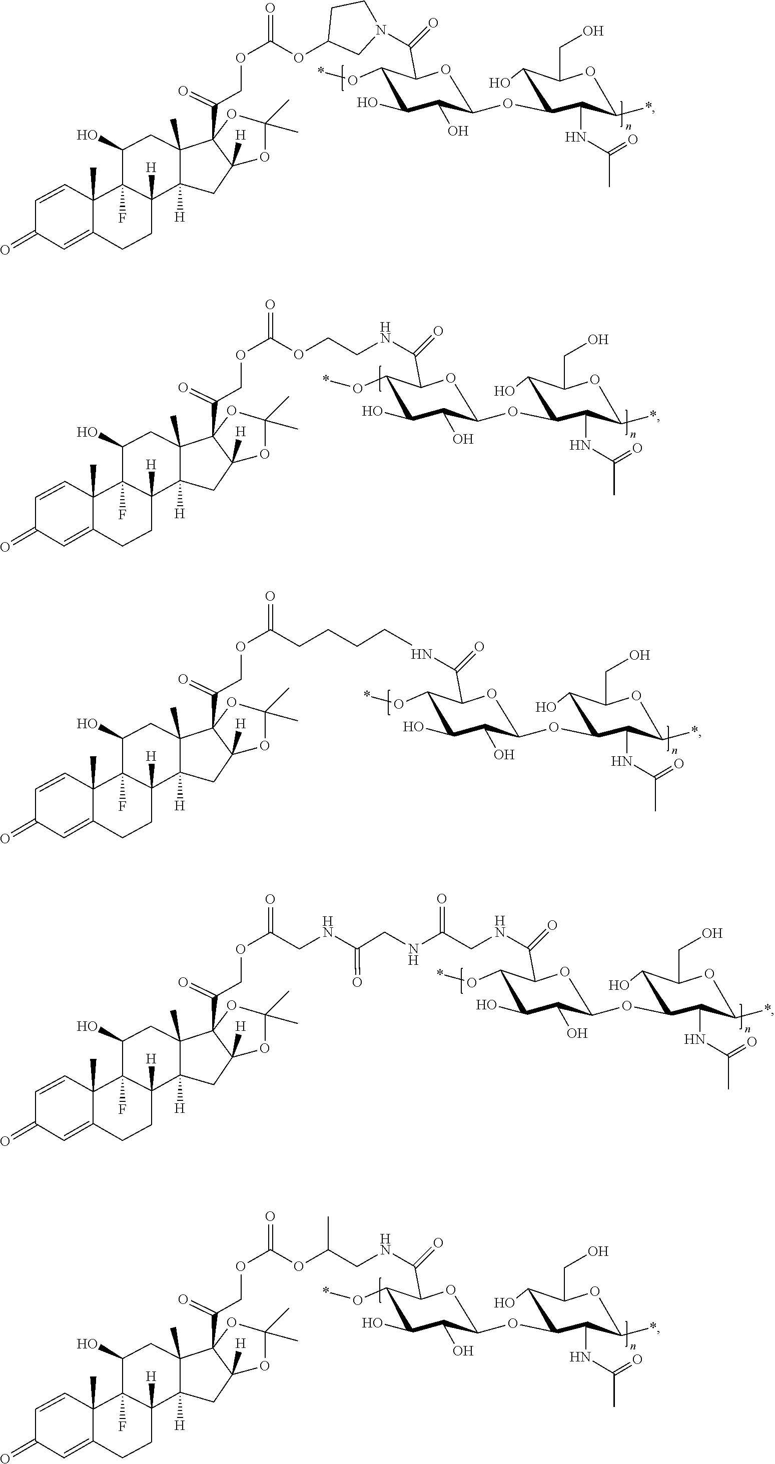

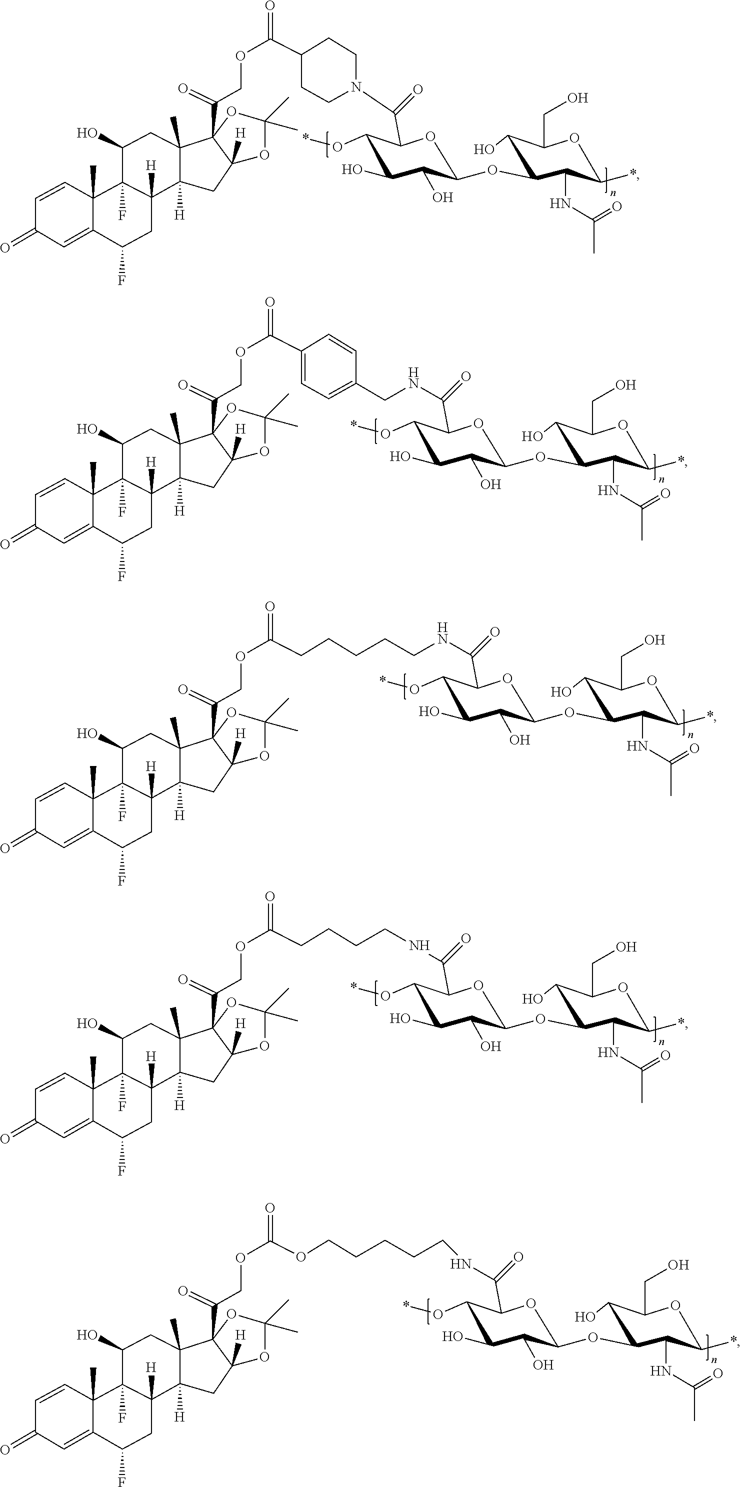

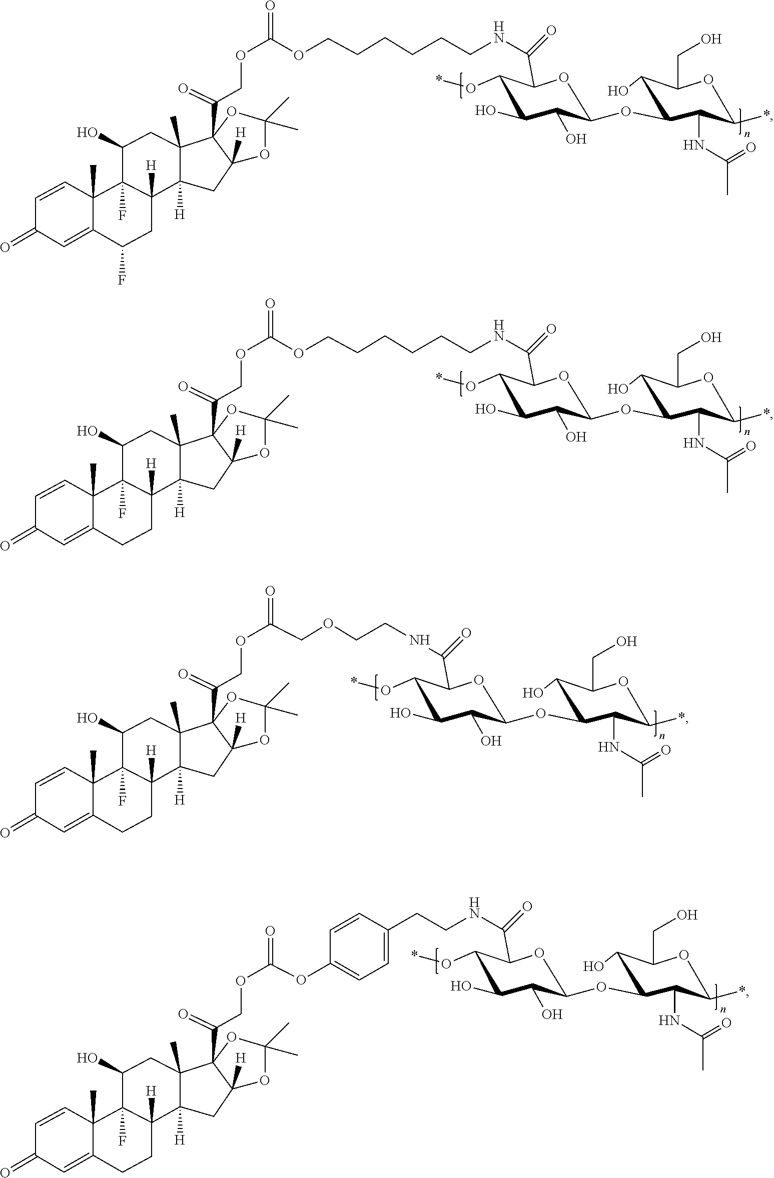

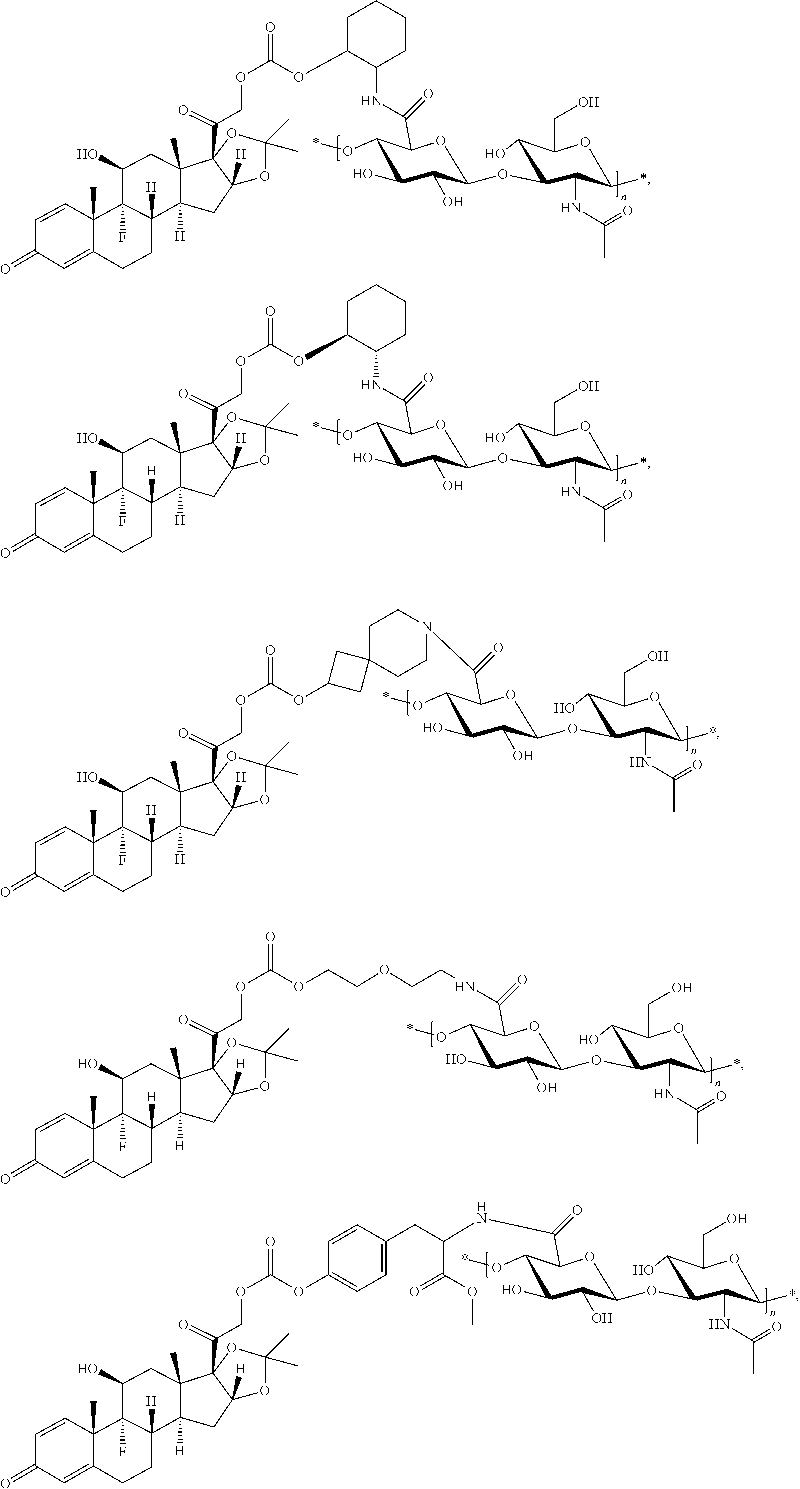

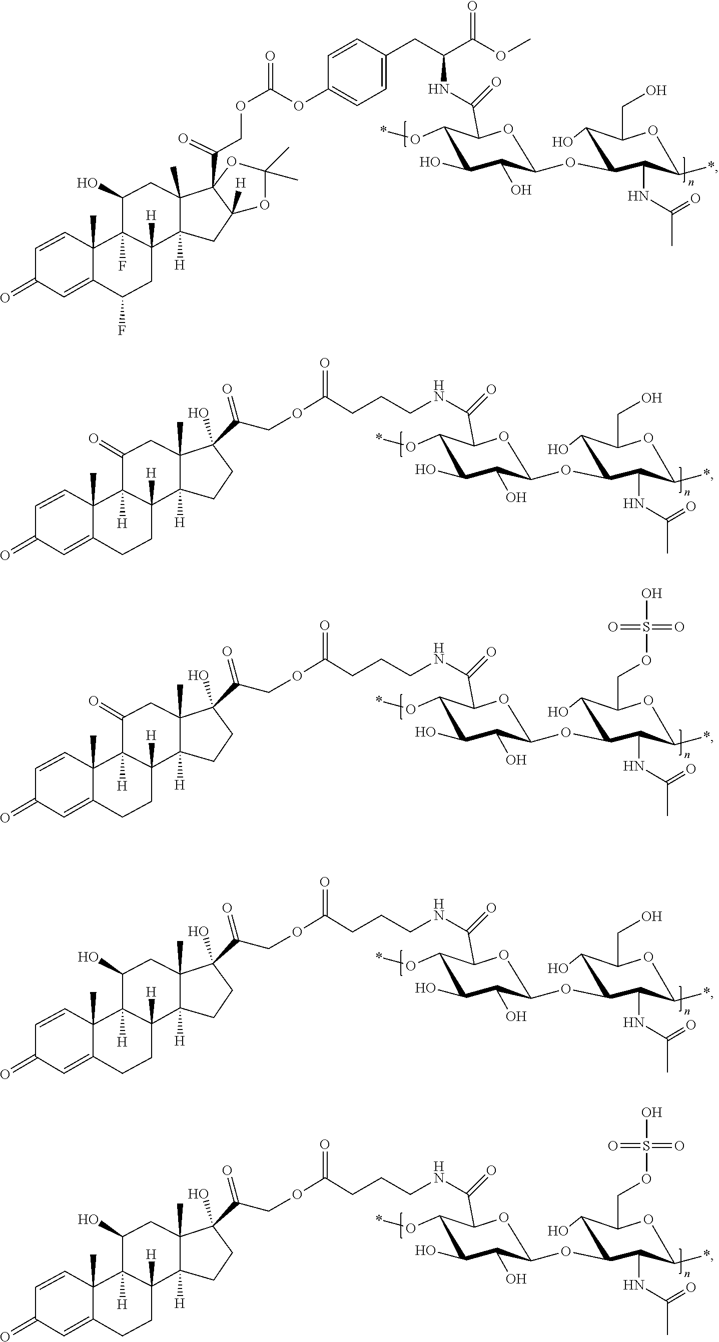

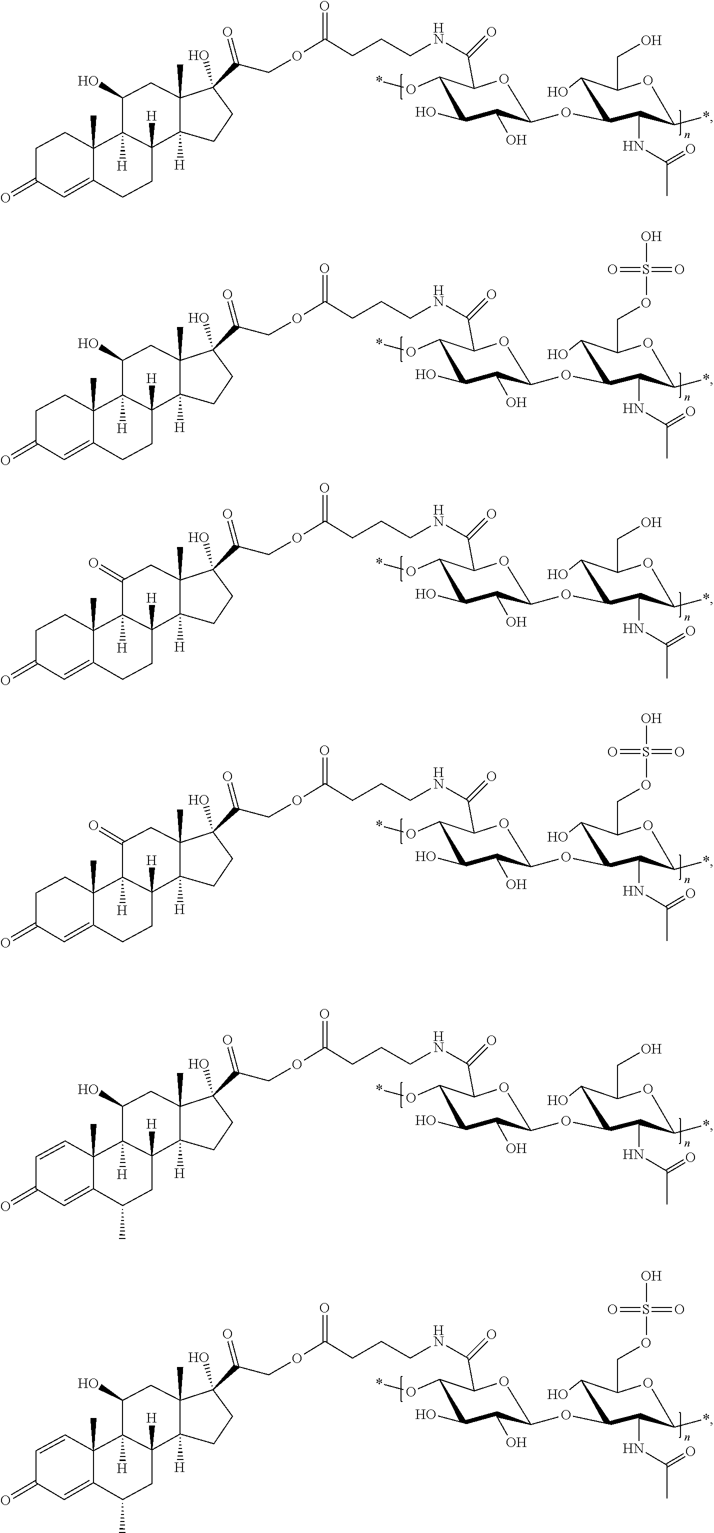

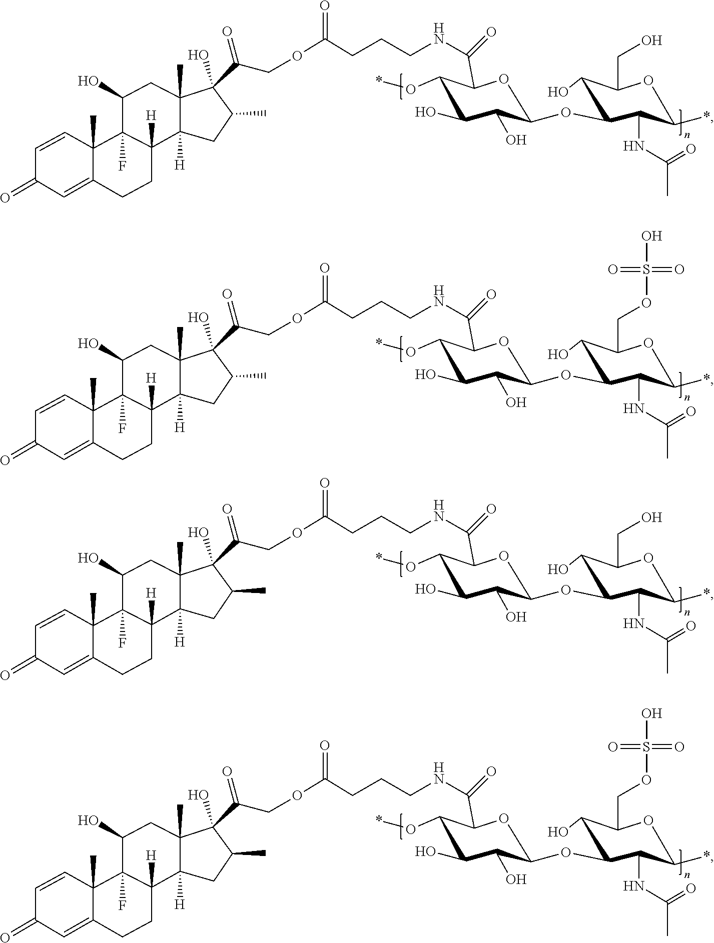

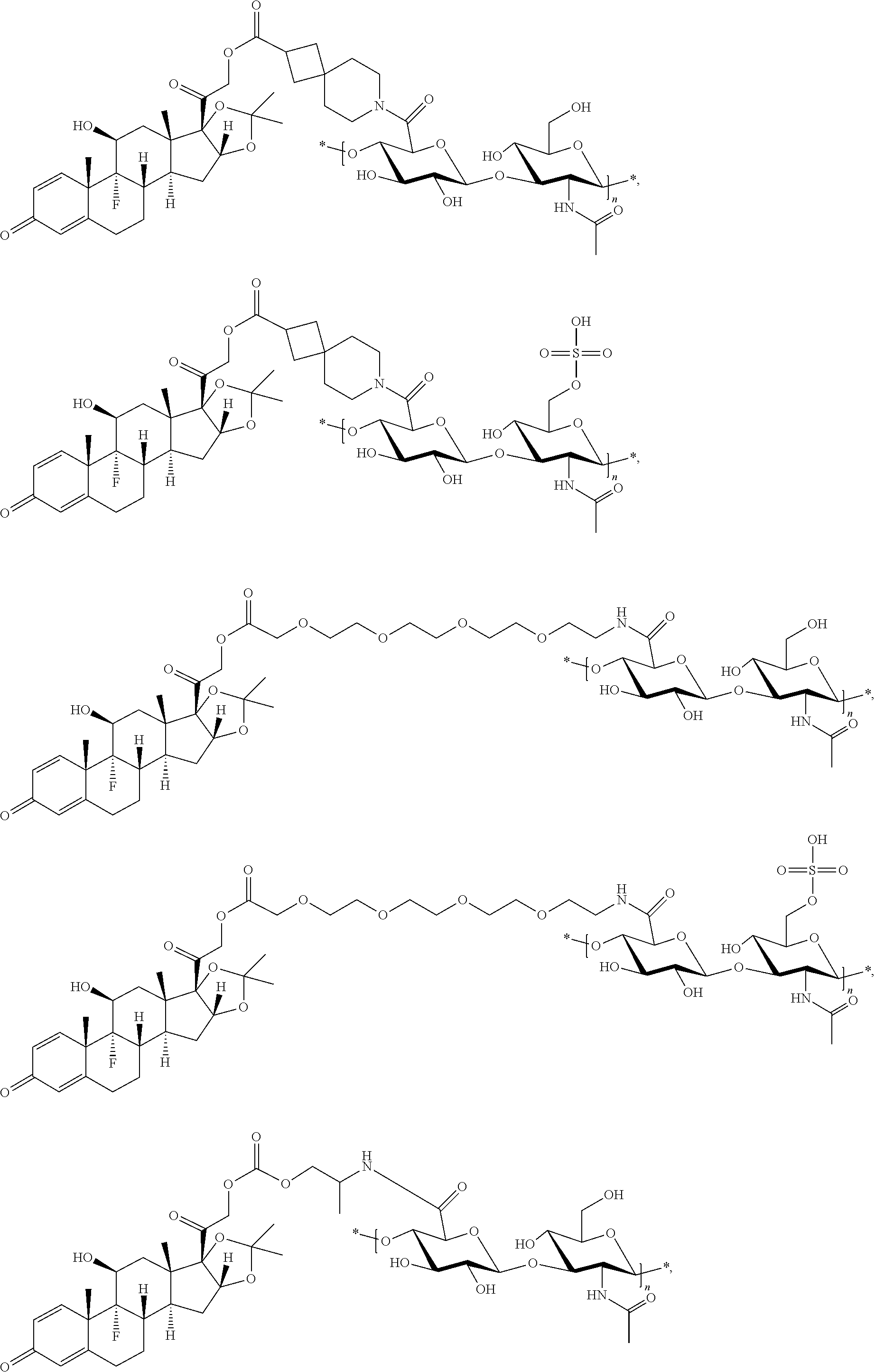

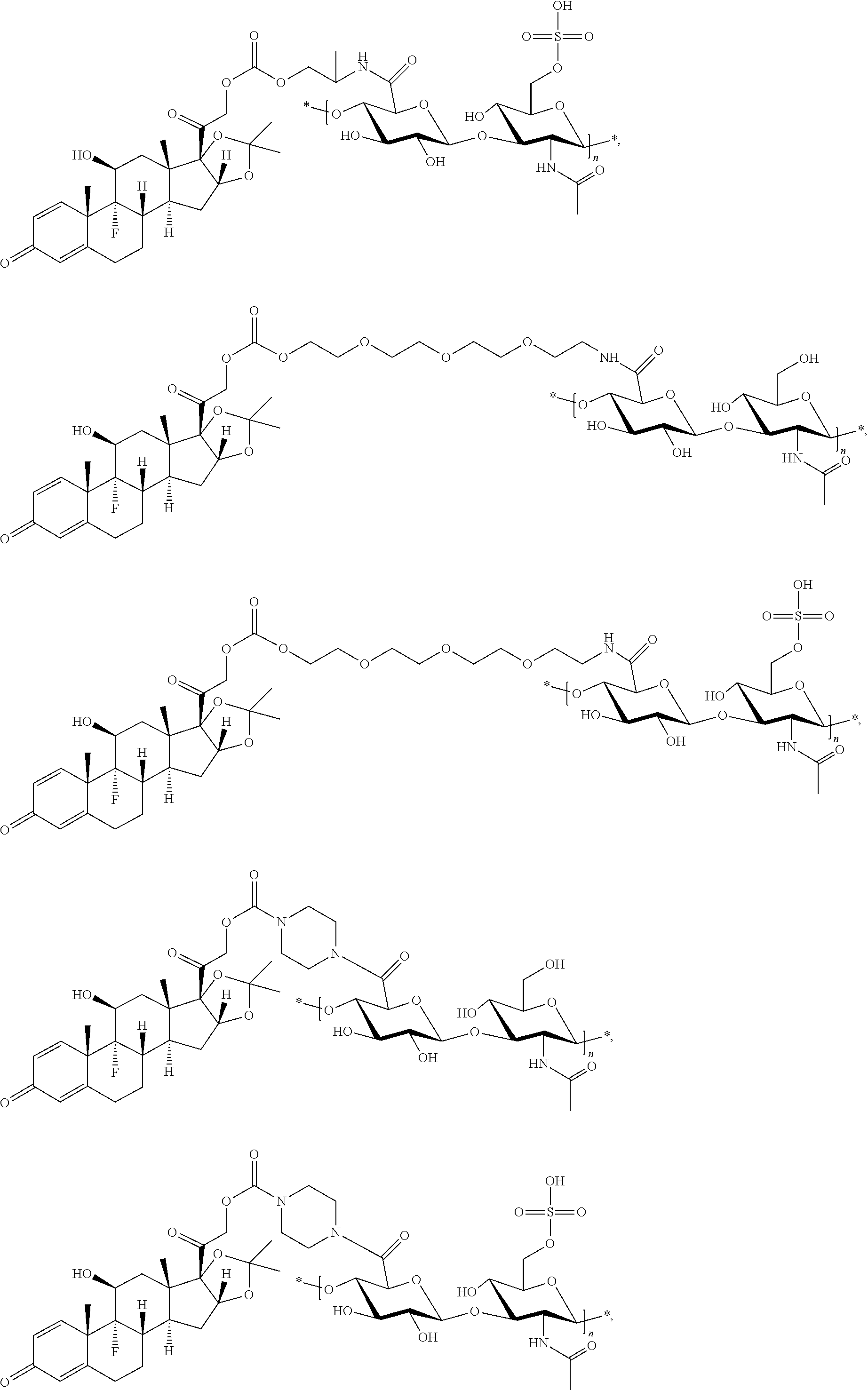

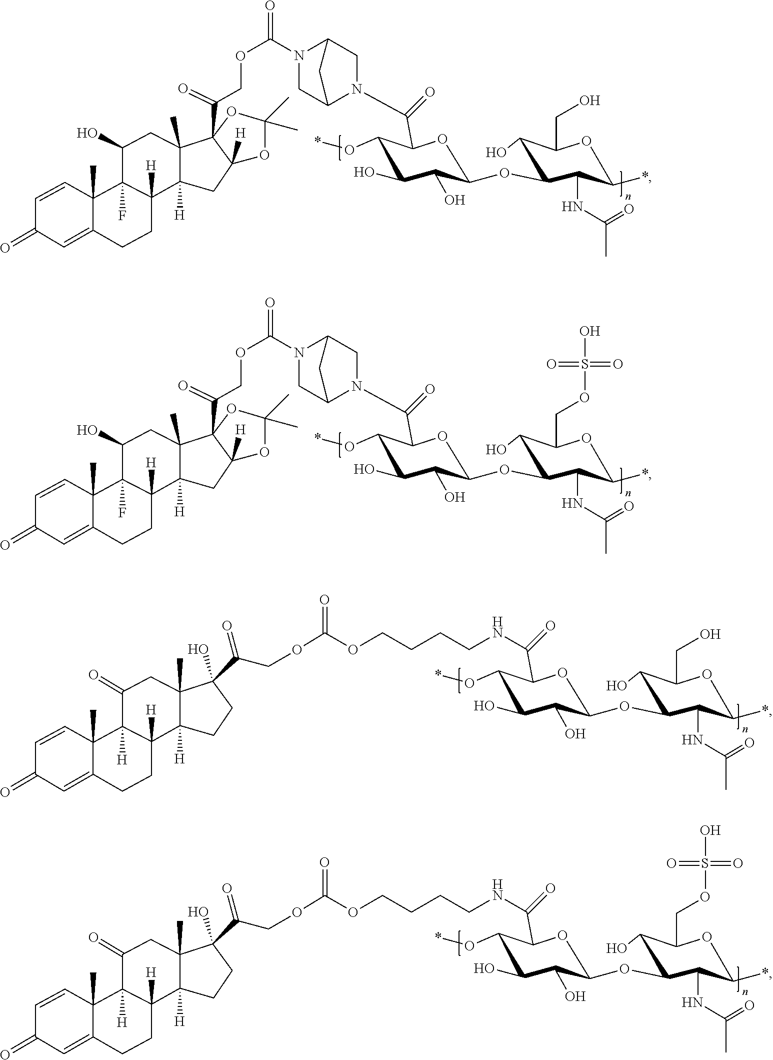

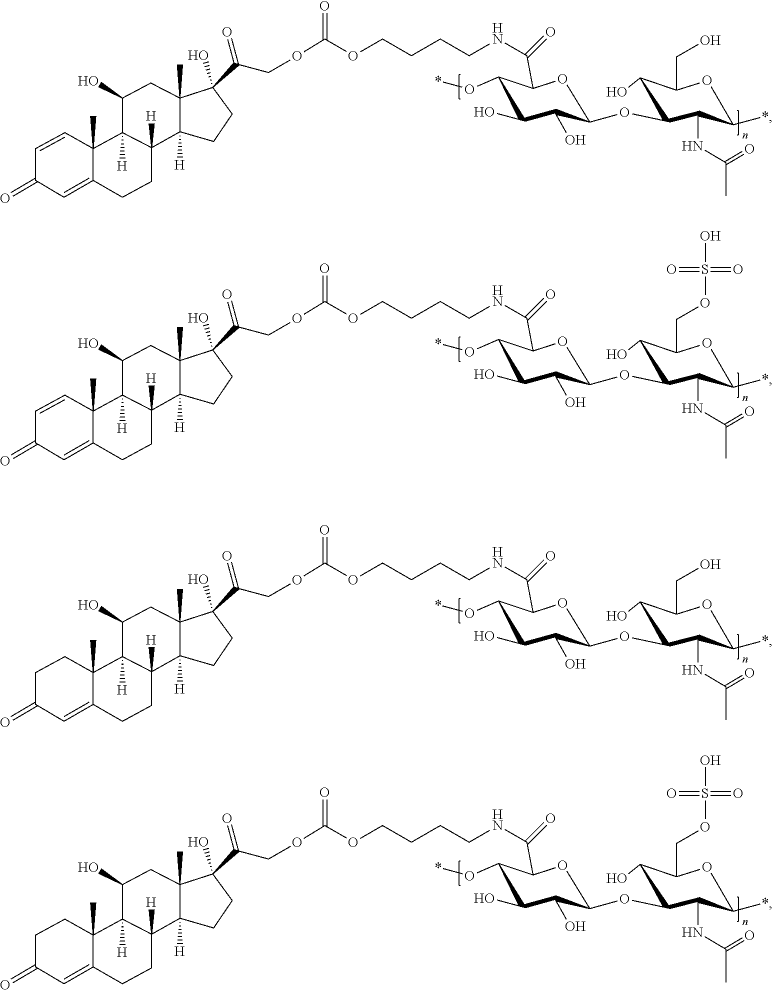

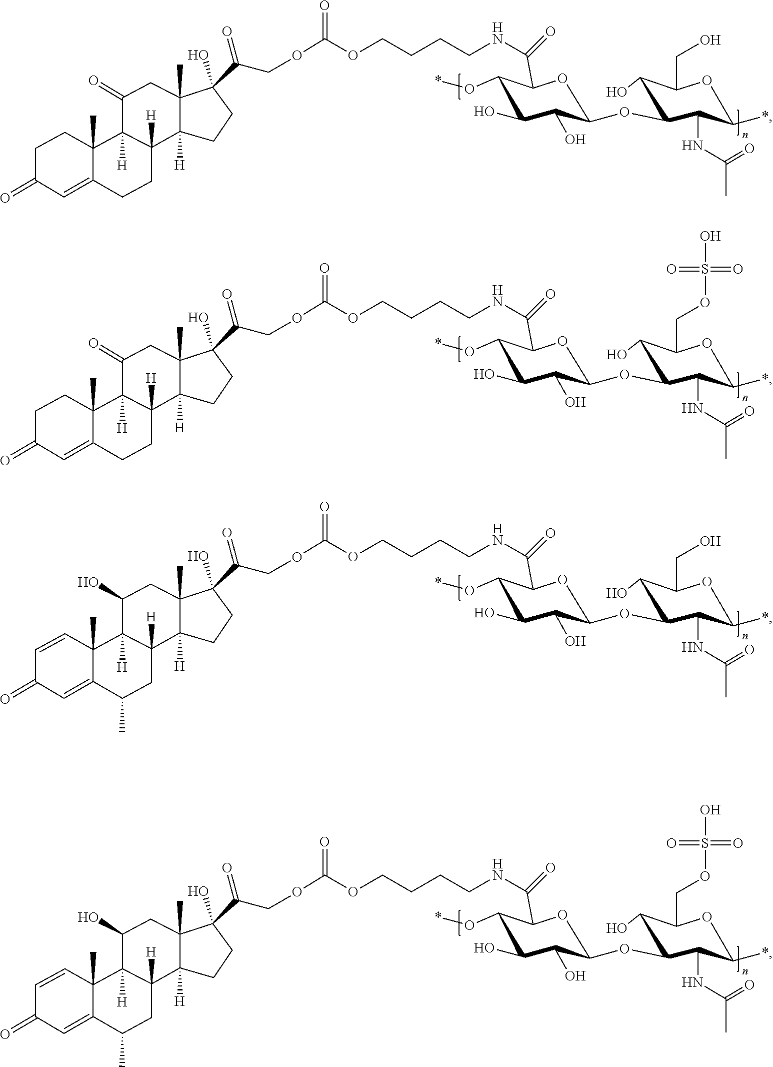

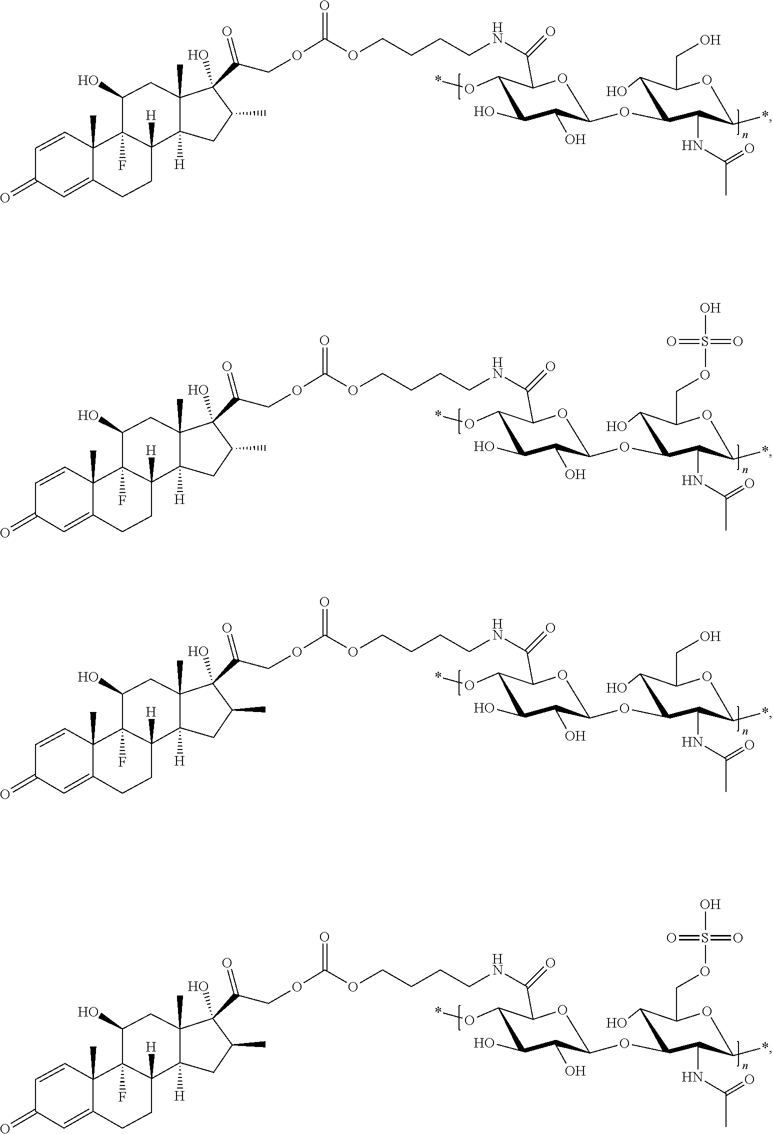

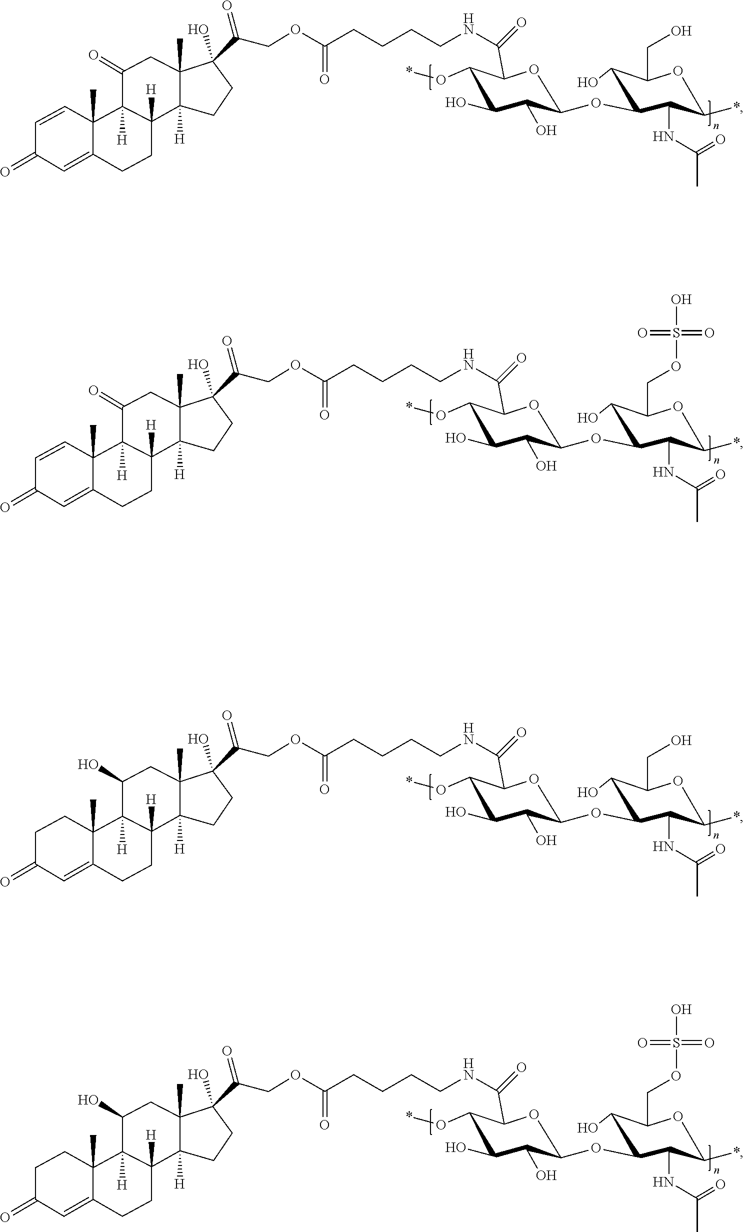

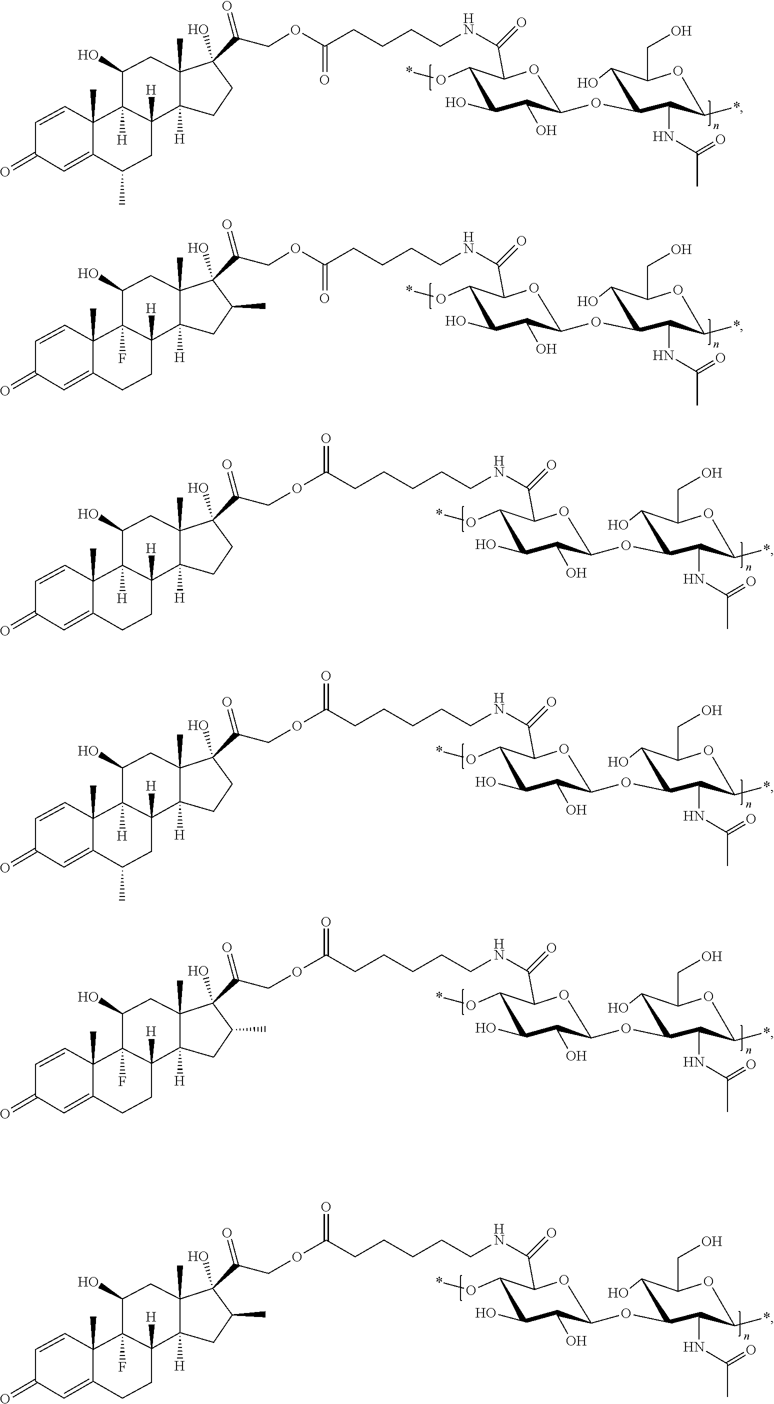

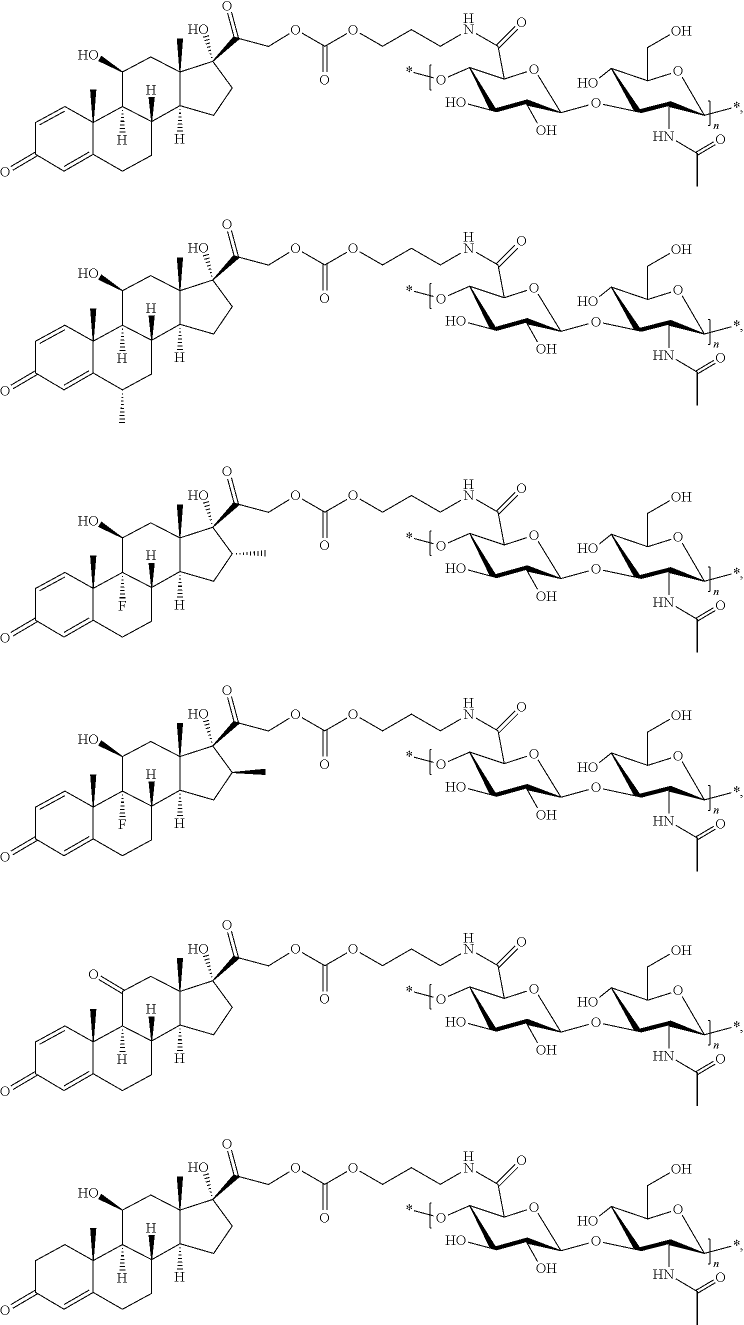

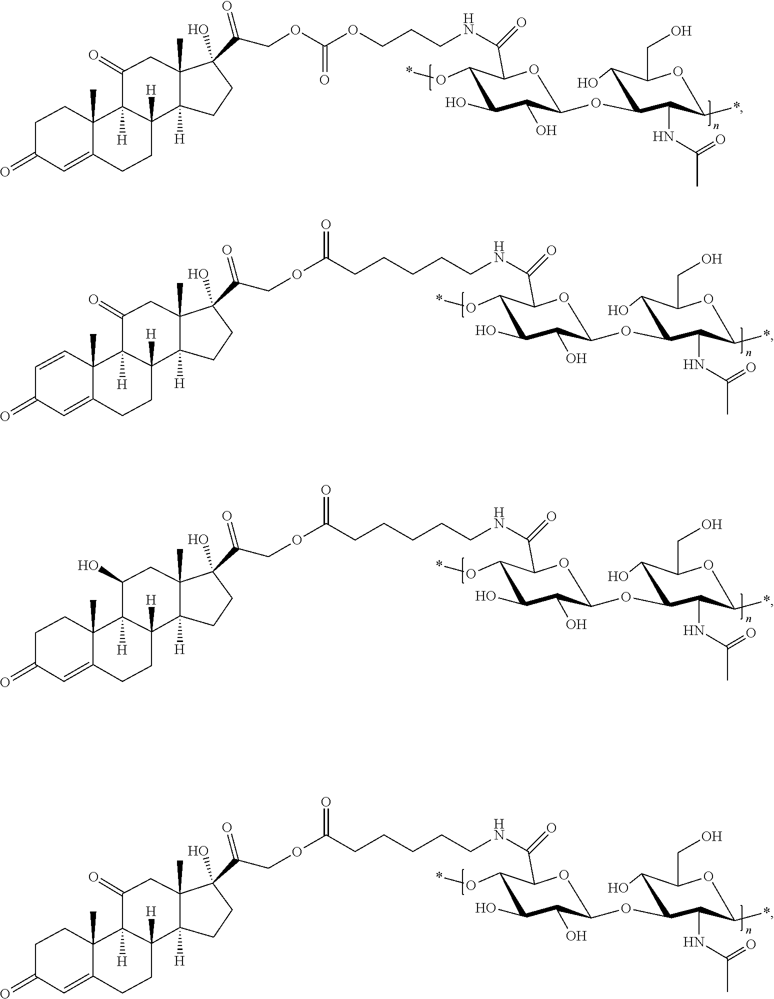

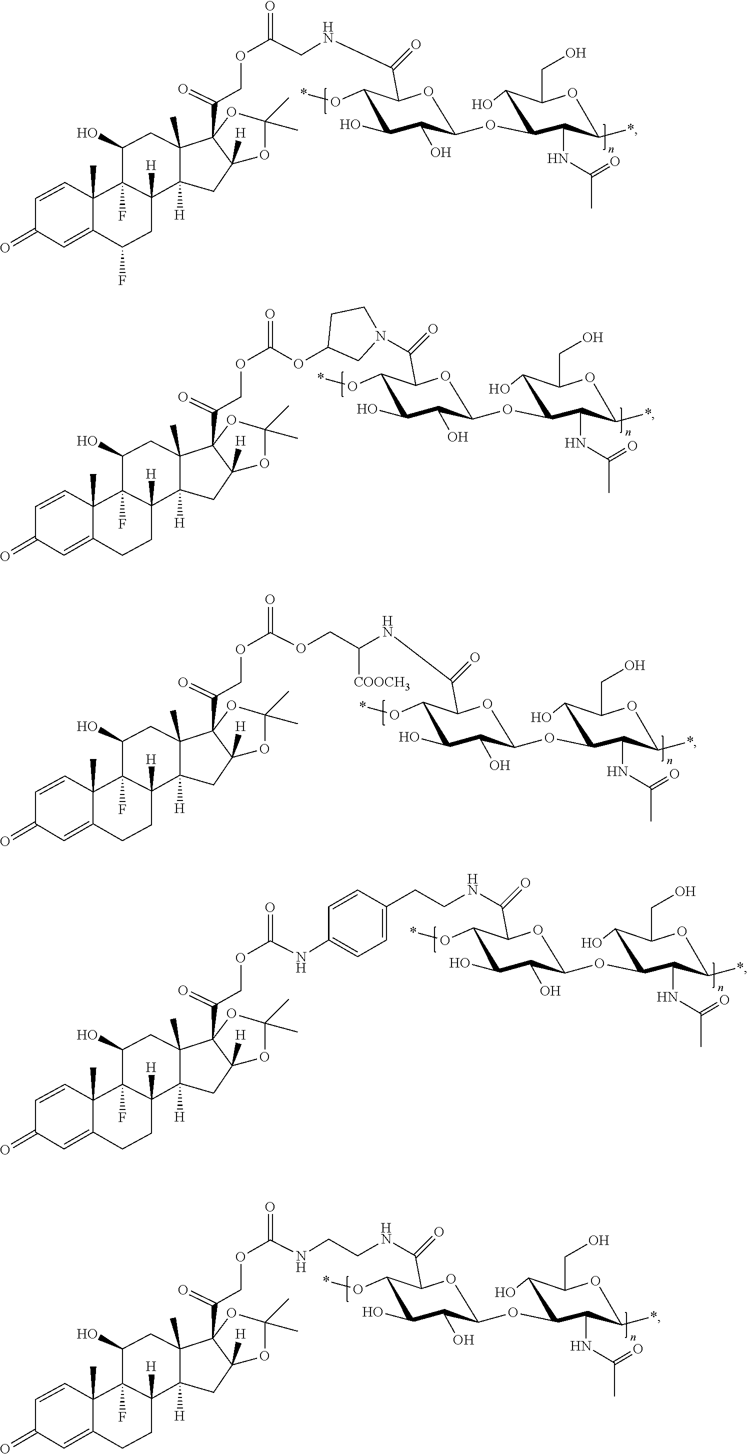

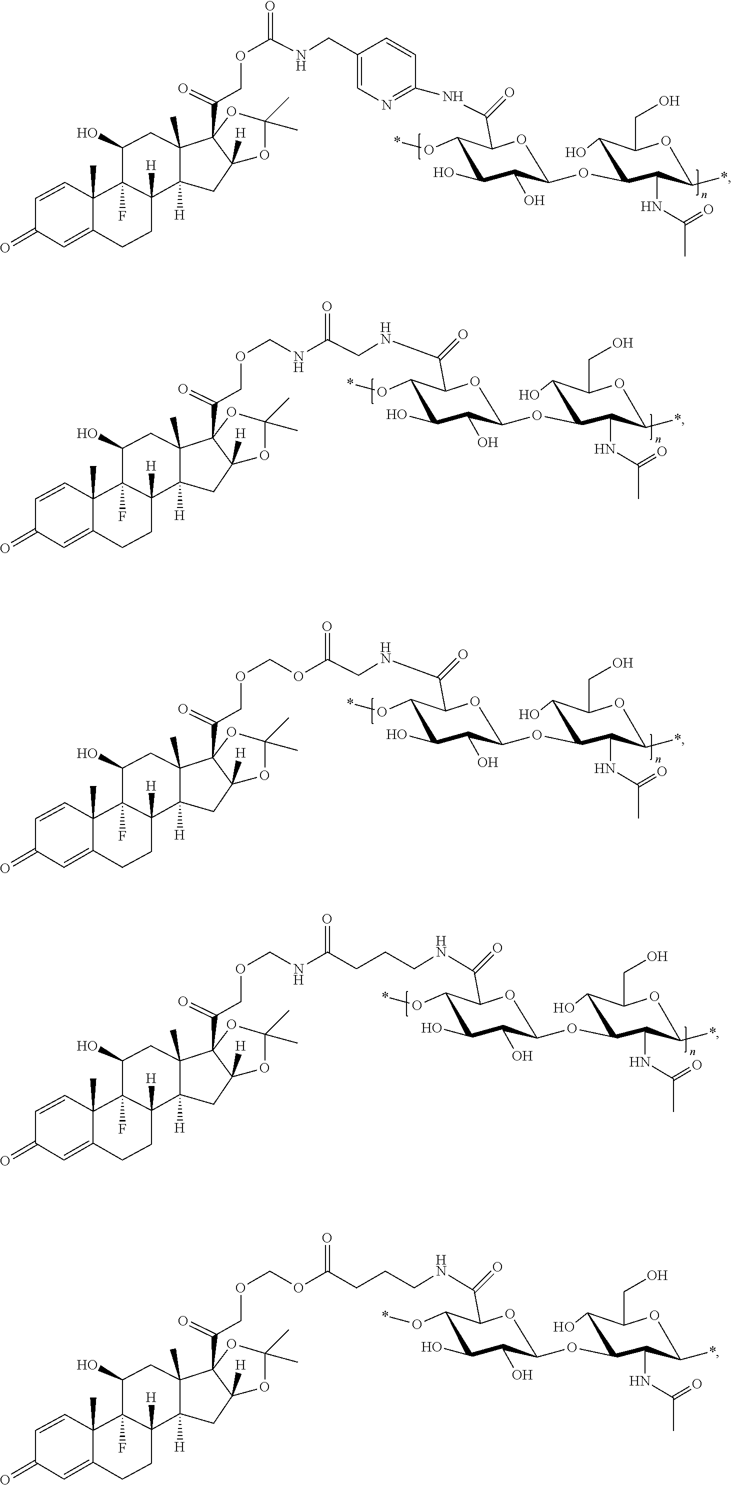

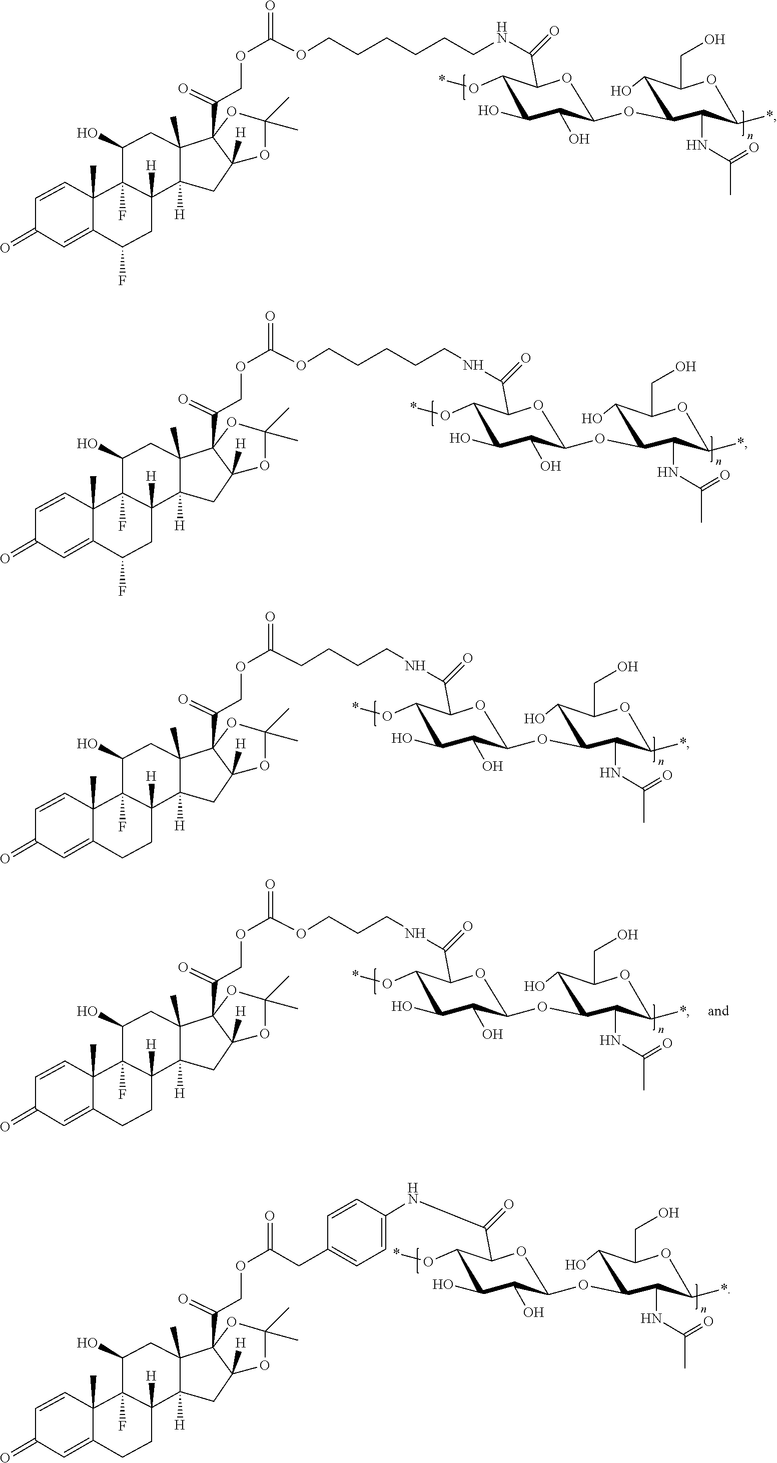

In some embodiments, the therapeutic agent can be conjugated to the biopolymer via the linker with a drug substitution rate to the biopolymer (DSR) as measured by NMR of at least 1%, at least 2%, at least 3%, at least 5%, at least 8%, at least 10%, at least 15%, at least 20%, at least 25%, at least 30%, at least 35%, at least 40%, at least 45%, at least 50%, wherein the drug substitution rate to the biopolymer (DSR) refers to the ratio of the molar amount of groups on the biopolymer which are substituted with drugs to the total molar amount of groups on the biopolymer which are capable of being substituted with drugs.

The therapeutic agent may be released from the drug delivery system provided herein through the cleavage of the linkage between the linker and the biopolymer or the therapeutic agent. In some embodiments, the release of the therapeutic agent occurs where the linkage between the biopolymer and the linker is cleaved to release a therapeutic agent-linker conjugate, which may be considered as a prodrug. Subsequent release of the therapeutic agent from the linker may involve enzymatic or non-enzymatic cleavage of the linkage between the therapeutic agent and the linker. In some embodiments, the release of the therapeutic agent occurs where the linkage between the therapeutic agent and the linker is cleaved without or prior to the cleavage of the linkage between the biopolymer and the linker. The release of the therapeutic agent may also involve enzymatic or non-enzymatic processes.

The release of therapeutic agents may be affected by a variety of factors, for example, the selection of specific therapeutic agent, linker and the biopolymer, the administration of the drug delivery system. The present disclosure contemplates biopolymers with varying molecular weight, binding group BG1, linkage with the linker; linkers with varying reactive functional groups and subunits; and therapeutic agents with varying binding group BG2, linkage with the linker.

The present disclosure also contemplates varying local administration of the drug delivery system provided herein. In some embodiments, the drug delivery system provided herein is locally administered to a subject in need thereof. In certain embodiments, the drug delivery system provided herein is locally administered to a subject in need thereof via injection. In certain embodiments, the drug delivery system provided herein is locally administered to a subject in need thereof via oral dosage form. In certain embodiments, the drug delivery system provided herein is locally administered to a subject in need thereof via inhalation. In certain embodiments, the drug delivery system provided herein is locally administered to a subject in need thereof via implant. In certain embodiments, the drug delivery system provided herein is locally administered to a subject in need thereof via topical application. Depending on the specific therapeutic agent, linker and the biopolymer combination, release of therapeutic agents may occur in a variety of locations upon administration to a subject. For example, release of therapeutic agents may occur at a site of administration.

In some embodiments, the administration of the drug delivery system provided herein to a subject may provide release of the therapeutic agent over a period of at least a few days to at least a few months.

The release of the therapeutic agent from the drug delivery system provided herein may be characterized by the percent of the therapeutic agent released per day from the drug delivery system. In some embodiments, the release rate of the therapeutic agent may vary in a range of about 0.01% to about 20% per day, about 0.01% to about 15% per day, about 0.01% to about 10% per day, about 0.01% to about 9% per day, about 0.01% to about 8% per day, about 0.01% to about 7% per day, about 0.01% to about 6% per day, about 0.01% to about 5% per day, about 0.01% to about 4% per day, about 0.01% to about 3% per day, about 0.01% to about 2% per day, about 0.01% to about 1% per day, about 0.01% to about 0.5% per day, about 0.01% to about 0.4% per day, about 0.01% to about 0.3% per day, about 0.01% to about 0.2% per day, about 0.01% to about 0.1% per day, about 0.01% to about 0.05% per day, about 0.01% to about 0.04% per day, about 0.01% to about 0.03% per day, or about 0.01% to about 0.02% per day.

### Pharmaceutical Compositions

In a further aspect, there is provided pharmaceutical compositions comprising the drug delivery system of the present disclosure.

In another aspect, there is provided pharmaceutical compositions comprising the drug delivery system of the present disclosure, and at least one pharmaceutical acceptable excipient.

As used herein, the term “pharmaceutical composition” refers to a formulation containing the drug delivery system of the present disclosure in a form suitable for administration to a subject.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used herein includes both one and more than one such excipient. The term “pharmaceutically acceptable excipient” also encompasses “pharmaceutically acceptable carrier” and “pharmaceutically acceptable diluent”.

The pharmaceutical compositions provided herein can be in any form that allows for the composition to be administered to a subject, including, but not limited to a human, and formulated to be compatible with an intended route of administration.

A variety of routes are contemplated for the pharmaceutical compositions provided herein, and accordingly the pharmaceutical composition provided herein may be supplied in bulk or in unit dosage form depending on the intended administration route. For example, for oral, buccal, and sublingual administration, powders, granules, tablets, pills, capsules, gelcaps, and caplets may be acceptable as solid dosage forms, and emulsions, syrups, elixirs, suspensions, and solutions may be acceptable as liquid dosage forms. For injection administration, gel, solutions, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form. For implant administration, solid, semi-solid, gel may be acceptable dosage form.

In some embodiments, the pharmaceutical compositions of the present disclosure may be in a form of formulation for oral administration.

In some embodiments, the pharmaceutical compositions of the present disclosure may be in a form of formulation for injection administration.

In some embodiments, the pharmaceutical compositions of the present disclosure may be in a form of formulation for inhalation administration.

In some embodiments, the pharmaceutical compositions of the present disclosure may be in a form of formulation for topical administration.

In certain embodiments, the pharmaceutical compositions provided herein may be formulated in the form of skin patches that are well known to those of ordinary skill in the art.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

In some embodiments, the pharmaceutical compositions of the present disclosure can be formulated as a single dose. The amount of the compounds provided herein in the single dose will vary depending on the subject treated and particular mode of administration.

In some embodiments, the pharmaceutical compositions of the present disclosure can be formulated to be administered to a subject at a time interval of a few days, a few weeks, a few months or even longer.

In a further aspect, there is also provided pharmaceutical compositions comprise the drug delivery system of the present disclosure, as two or more combination therapy.

### Synthesis of the Drug Delivery System

Synthesis of the drug delivery system provided herein is illustrated in the synthetic schemes in the examples. The drug delivery system provided herein can be prepared using any known organic synthesis techniques and can be synthesized according to any of numerous possible synthetic routes, and thus these schemes are illustrative only and are not meant to limit other possible methods that can be used to prepare the compounds provided herein. Additionally, the steps in the schemes are for better illustration and can be changed as appropriate. The embodiments of the compounds in examples were synthesized for the purposes of research and potentially submission to regulatory agencies.

The reactions for preparing the drug delivery system of the present disclosure can be carried out in suitable solvents, which can be readily selected by one skilled in the art of organic synthesis. Suitable solvents can be substantially non-reactive with the starting materials (reactants), the intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G. M. Wuts, Greene's Protective Groups in Organic Synthesis, 5^{th} Edition, Wiley, 2014, all of which are incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

The known starting materials of the present disclosure can be synthesized by using or according to the known methods in the art, or can be purchased from commercial suppliers. Unless otherwise noted, analytical grade solvents and commercially available reagents were used without further purification.

Unless otherwise specified, the reactions of the present disclosure were all done under a positive pressure of nitrogen or argon or with a drying tube in anhydrous solvents, and the reaction flasks were typically fitted with rubber septa for the introduction of substrates and reagents via syringe. Glassware was oven dried and/or heat dried.

### Method of Treatment of Disease

In a further aspect, there is provided a method of treating a disorder in a subject in need thereof, comprising administering to the subject a therapeutic effective amount of the drug delivery system or the pharmaceutical composition provided herein.

The disorder to be treated depends on the selected therapeutic agent in the drug delivery system or the pharmaceutical composition provided herein. In some embodiments, the disorder can be allergic diseases, autoimmune diseases, or inflammatory diseases. In certain embodiments, the disorder can be selected from the group consisting of allergic rhinitis, systemic lupus erythematosus, rheumatism, nephrotic syndrome, autoimmune hemolytic anemia, idiopathic thrombocytopenic purpura, Addison disease, neurodermatitis, cutaneous pruritus, tendinitis, inflammation, respiratory disease, osteoarthritis, Neovascular (Wet) Age-Related Macular Degeneration (AMD), Macular Edema Following Retinal Vein Occlusion (RVO), Diabetic Macular Edema (DME), Uveitic Macular Edema (UME), Diabetic Retinopathy (DR), Myopic Choroidal Neovascularization (mCNV), dermatitis, psoriasis, chronic obstructive pulmonary disease, and asthma.

In this context, the term “therapeutically effective amount” refers to an amount of a therapeutic agent selected in the drug delivery system provided herein or pharmaceutically acceptable salts thereof which is effective to provide “therapy” in a subject, or to “treat” discorders, diseases or conditions in a subject.

### EXAMPLES

For the purpose of illustration, the following examples are included. However, it is to be understood that these examples do not limit the present disclosure and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents and building blocks known in the art other than those described, and/or by making routine modifications of reaction conditions. Alternatively, other reactions disclosed herein or known in the art will be recognized as having applicability for preparing other compounds of the present disclosure.

### Example 1

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl glycinate

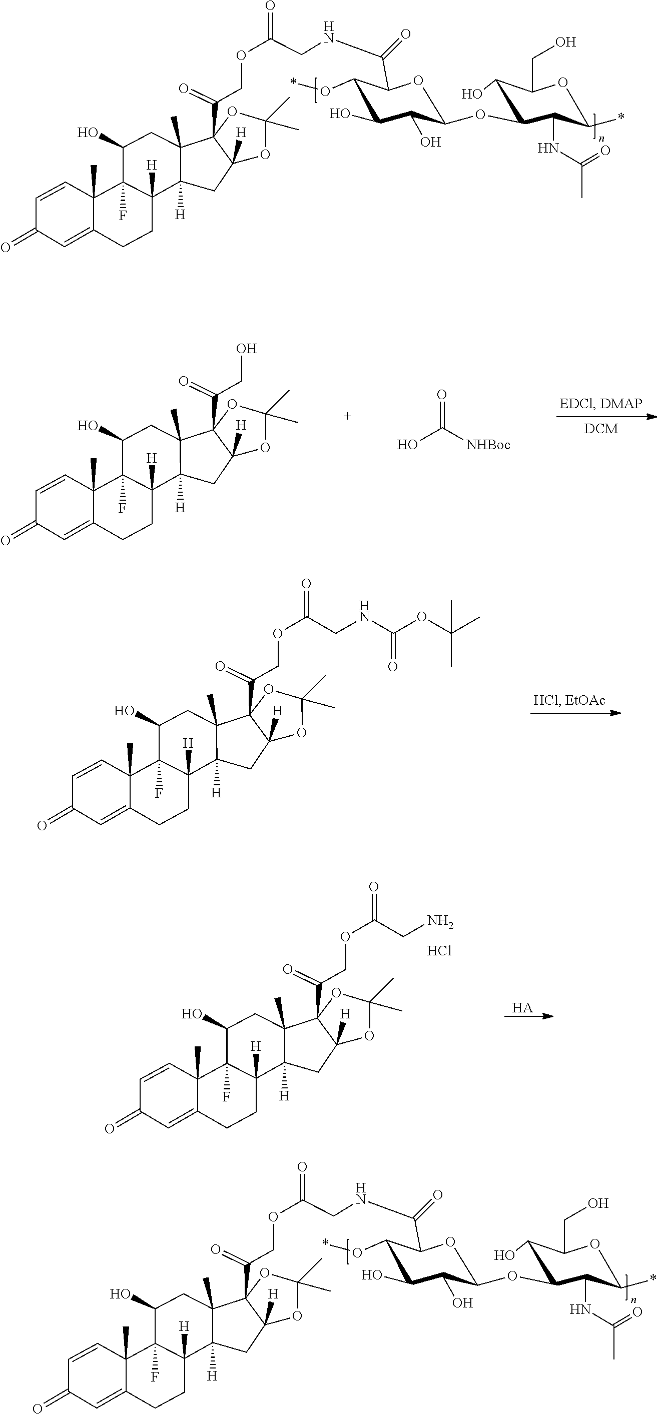

### Step 1: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (tert-butoxycarbonyl)glycinate

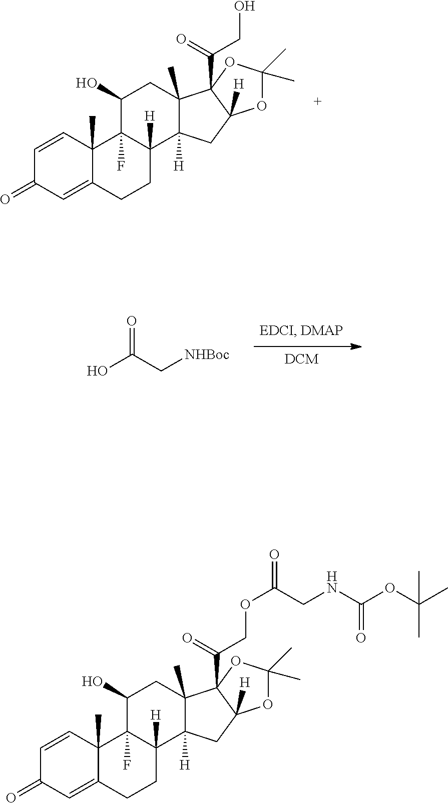

9α-Fluoro-11β,16α,17,21-tetrahydroxy-1,4-pregnadiene-3,20-dione (Triamcinolone acetonide, 1086 mg, 2.5 mmol), (tert-butoxycarbonyl)glycine (438 mg, 2.5 mmol), 1-ethyl-3-(3-dimethylaminopropyl)carbodiimide (EDCI, 527 mg, 2.75 mmol), N,N-dimethylpyridin-4-amine (DMAP, 31 mg, 0.25 mmol) were dissolved in DCM (25 mL). The reaction mixture was stirred at room temperature for 3 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=2:1˜1:1) to afford the title compound (1120 mg, Yield: 77.3%). MS (m/z): [M+H]+ calcd for C₃₁H₄₂FNO₉, 592.67; found, 536.2 (M+H−56). ¹H NMR (400 MHz, Chloroform-d) δ 7.21 (d, J=10.2 Hz, 1H), 6.35 (dd, J=10.1, 1.9 Hz, 1H), 6.13 (t, J=1.7 Hz, 1H), 5.05 (d, J=9.4 Hz, 1H), 5.01-4.87 (m, 3H), 4.42 (d, J=8.9 Hz, 1H), 4.11-3.97 (m, 2H), 2.63 (tdd, J=13.9, 6.1, 1.8 Hz, 1H), 2.54-2.29 (m, 4H), 2.17-2.05 (m, 1H), 1.86 (dt, J=11.8, 5.2 Hz, 1H), 1.77-1.59 (m, 4H), 1.55 (s, 3H), 1.44 (d, J=9.8 Hz, 12H), 1.21 (s, 3H), 0.93 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl glycinate hydrochloride

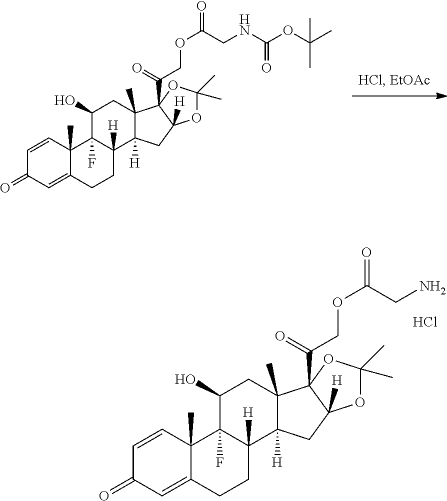

To a stirred solution of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (tert-butoxycarbonyl)glycinate (500 mg, 0.85 mmol) in EtOAc (10 mL) was slowly added 4M HCl in ethyl acetate (2 mL) at ice-bath. The reaction mixture was allowed to stir at room temperature for 18 hours. The solution was diluted with EtOAc and concentrated under reduced pressure to afford the title compound as a white solid (0.43 g, yield: 80.6%) that was used without further purification. MS (m/z): [M+H]+ calcd for C₂₆H₃₄FNO₇, 492.56; found, 492.3.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl glycinate

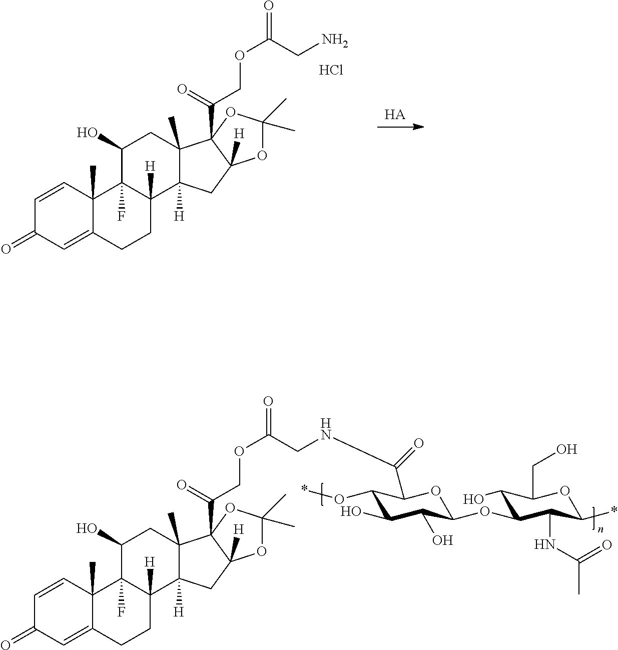

Hyaluronic acid (201.5 mg, 0.5 mmol carboxylic acid) was dissolved in 40 mL of deionized water in a 100 mL round-bottomed flask followed by the dropwise addition of 26 mL of acetonitrile while stirring. To the solution was added 4-methylmorpholine (NMM, 35 mg, 0.35 mmol) and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl glycinate hydrochloride (185 mg, 0.35 mmol) causing the viscosity to increase temporarily. The solution was then cooled to 0° C., and 4-(4,6-Dimethoxy-1,3,5-triazin-2-yl)-4-methylmorpholinium chloride (DMTMM, 111 mg, 0.4 mmol) was added and stirred at room temperature for 72 hours at room temperature. NaCl (293 mg, 5 mmol) was then added to the reaction mixture, which was stirred for 1 hour and followed by the dropwise addition of anhydrous alcohol (200 mL) while stirring. The mixture was filtered. The filter cake was collected, washed with anhydrous alcohol and dried under vacuo to give the title compound as a white solid. (sodium hyaluronate MW 50 KDa 0.255 g, yield: 68.3%, DSR=32%); ¹H NMR (400 MHz, Deuterium Oxide) δ 7.69-7.56 (m, 0.32H), 6.56-6.45 (m, 0.32H), 6.35-6.27 (m, 0.32H), 5.51-5.35 (m, 0.32H), 5.19-5.00 (m, 0.64H), 4.77-4.36 (m, 2.32H), 4.28-3.15 (m, 10.64H), 2.90-2.45 (m, 1.28H), 2.31-2.18 (m, 0.32H), 2.17-1.73 (m, 4.28H), 1.69-1.47 (m, 1.92H), 1.41-1.26 (m, 0.96H), 1.08-0.89 (m, 0.96H).

With This procedure, reaction of sodium hyaluronate (MW 500 KDa) provided corresponding product (0.2 g, yield: 53.6%, DSR=33%). ¹H NMR (400 MHz, Deuterium Oxide) δ 7.69-7.55 (m, 0.33H), 6.57-6.45 (m, 0.33H), 6.35-6.25 (m, 0.33H), 5.51-5.34 (m, 0.33H), 5.18-5.01 (m, 0.66H), 4.77-4.36 (m, 2.33H), 4.29-3.17 (m, 10.66H), 2.89-2.46 (m, 1.32H), 2.30-2.19 (m, 0.33H), 2.17-1.71 (m, 4.32H), 1.65-1.44 (m, 1.98H), 1.38-1.23 (m, 0.99H), 1.06-0.88 (m, 0.99H).

With this procedure, reaction of sodium hyaluronate (MW 2000 KDa) provided corresponding product (0.21 g, yield: 56.3%, DSR=30%). ¹H NMR (400 MHz, Deuterium Oxide) δ 7.67-7.55 (m, 0.3H), 6.56-6.45 (m, 0.3H), 6.36-6.27 (m, 0.3H), 5.51-5.34 (m, 0.3H), 5.18-5.01 (m, 0.6H), 4.78-4.35 (m, 2.3H), 4.31-3.22 (m, 10.6H), 2.89-2.46 (m, 1.2H), 2.33-2.19 (m, 0.3H), 2.18-1.73 (m, 4.2H), 1.70-1.48 (m, 1.8H), 1.40-1.26 (m, 0.9H), 1.10-0.90 (m, 0.9H).

### Example 2

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-aminobutanoate

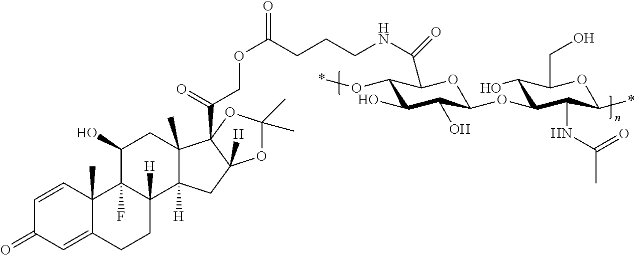

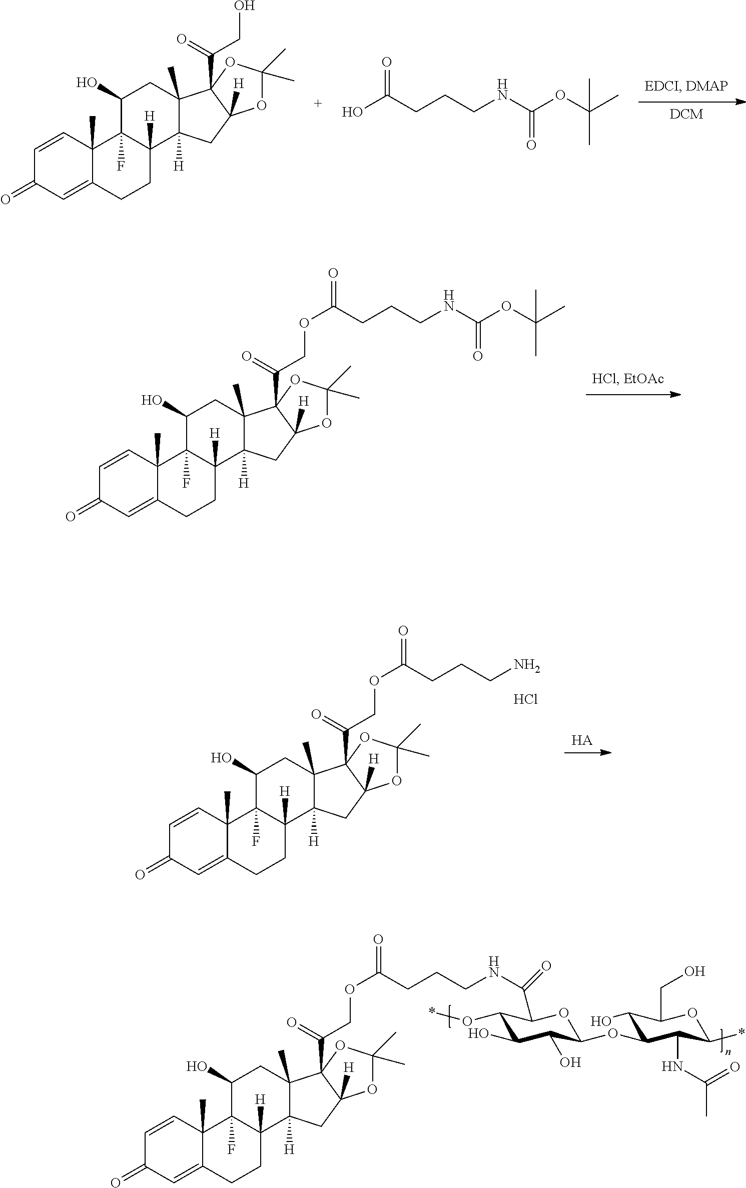

### Step 1: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-((tert-butoxycarbonyl)amino)butanoate

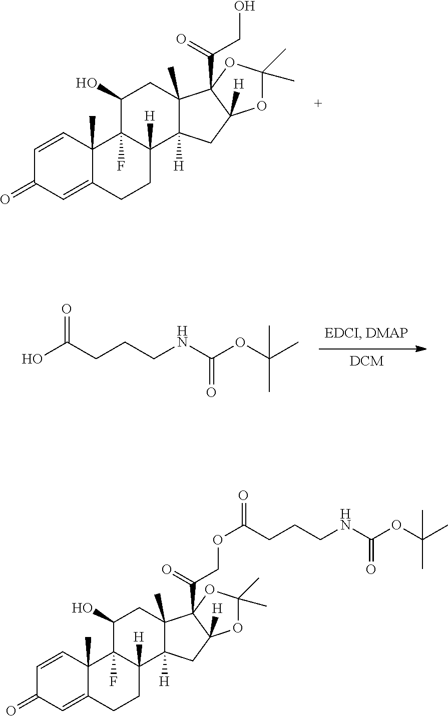

9α-Fluoro-11β,16α,17,21-tetrahydroxy-1,4-pregnadiene-3,20-dione (Triamcinolone acetonide, 4345 mg, 10 mmol), 4-((tert-butoxycarbonyl)amino)butanoic acid (2642 mg, 13 mmol), 1-ethyl-3-(3-dimethylaminopropyl)carbodiimide (EDCI, 2492 mg, 13 mmol), N,N-dimethylpyridin-4-amine (DMAP, 122 mg, 1 mmol) were dissolved in DCM (100 mL). The reaction mixture was stirred at room temperature for 24 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=5:1˜2:1) to afford the title compound (6000 mg, Yield: 96.9%). MS (m/z): [M+H]⁺ calcd for C₃₃H₄₆FNO₉, 620.73; found, 564.3 (M+H−56). ¹H NMR (400 MHz, Chloroform-d) δ 7.20 (d, J=10.1 Hz, 1H), 6.35 (dd, J=10.2, 1.9 Hz, 1H), 6.13 (d, J=1.8 Hz, 1H), 4.98 (d, J=4.9 Hz, 1H), 4.90 (s, 2H), 4.68 (s, 1H), 4.42 (d, J=8.7 Hz, 1H), 3.20 (q, J=6.7 Hz, 2H), 2.62 (tt, J=14.2, 7.3 Hz, 1H), 2.54-2.35 (m, 5H), 2.17-2.06 (m, 1H), 1.92-1.82 (m, 3H), 1.74-1.58 (m, 5H), 1.55 (s, 3H), 1.44 (d, J=4.0 Hz, 12H), 1.21 (s, 3H), 0.94 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-aminobutanoate hydrochloride

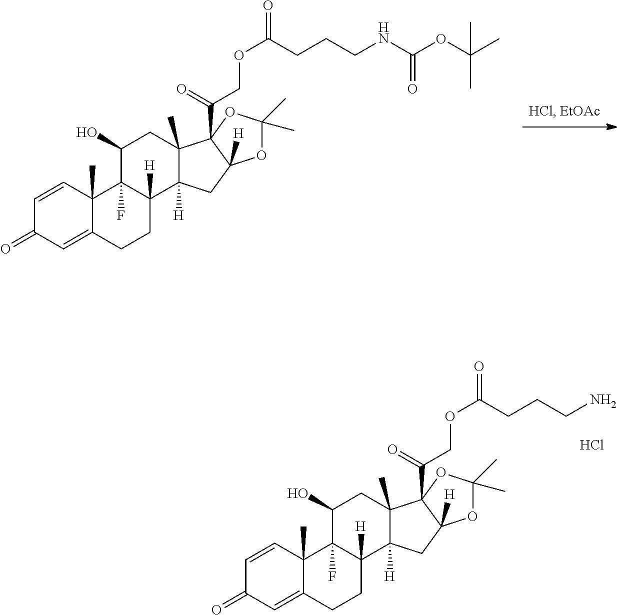

To a stirred solution of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-((tert-butoxycarbonyl)amino)butanoate (600 mg, 0.97 mmol) in EtOAc (24 mL) was slowly added 4M HCl in ethyl acetate (4.8 mL) at ice-bath. The reaction mixture was allowed to stir at room temperature for 18 hours. The solution was diluted with EtOAc and concentrated under reduced pressure to afford the title compound as a white solid (0.53 g, yield: 98.3%) that was used without further purification. MS (m/z): [M+H]+ calcd for C₂₈H₃₈FNO₇, 520.61; found, 520.1.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-aminobutanoate

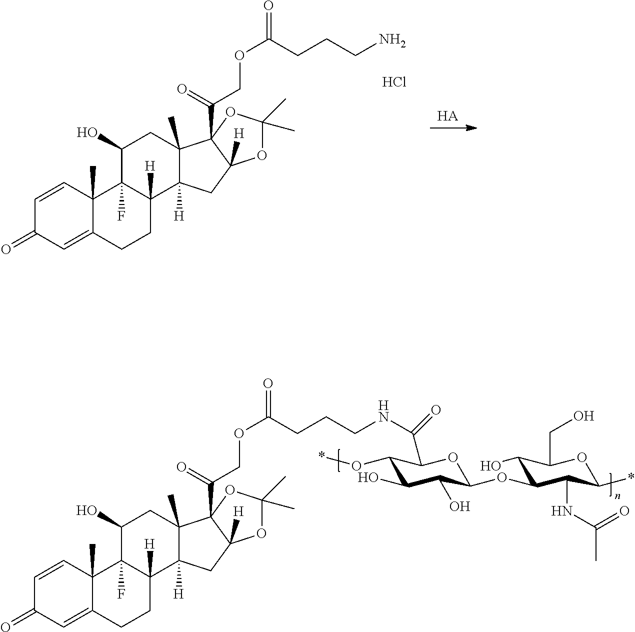

Hyaluronic acid (201.5 mg, 0.5 mmol carboxylic acid) was dissolved in 40 mL of deionized water in a 100 mL round-bottomed flask followed by the dropwise addition of 26 mL of acetonitrile while stirring. To the solution was added 4-methylmorpholine (NMM, 35 mg, 0.35 mmol) and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-aminobutanoate hydrochloride (195 mg, 0.35 mmol) causing the viscosity to increase temporarily. The solution was then cooled to 0° C., and 4-(4,6-Dimethoxy-1,3,5-triazin-2-yl)-4-methylmorpholinium chloride (DMTMM, 111 mg, 0.4 mmol) was added and stirred at room temperature for 72 hours at room temperature. NaCl (293 mg, 5 mmol) was then added to the reaction mixture, which was stirred for 1 hour and followed by the dropwise addition of anhydrous alcohol (200 mL) while stirring. The mixture was filtered. The filter cake was collected, washed with anhydrous alcohol and dried under vacuo to give the title compound as a white solid. (sodium hyaluronate MW 50 KDa 0.263 g, yield: 68.6%, DSR=32%); ¹H NMR (400 MHz, Deuterium Oxide) δ 7.70-7.56 (m, 0.32H), 6.59-6.47 (m, 0.32H), 6.39-6.26 (m, 0.32H), 5.43-5.31 (m, 0.32H), 5.20-5.01 (m, 0.64H), 4.78-4.44 (m, 2.32H), 4.21-3.16 (m, 10.64H), 2.91-2.51 (m, 1.6H), 2.35-2.26 (m, 0.32H), 2.22-1.72 (m, 5.56H), 1.70-1.47 (m, 1.92H), 1.41-1.25 (m, 0.96H), 1.06-0.92 (m, 0.96H).

With this procedure, reaction of sodium hyaluronate (MW 500 KDa) provided corresponding product (0.196 g, yield: 51.1%, DSR=30%). ¹H NMR (400 MHz, Deuterium Oxide) δ 7.70-7.56 (m, 0.3H), 6.58-6.48 (m, 0.3H), 6.38-6.26 (m, 0.3H), 5.42-5.28 (m, 0.3H), 5.18-5.00 (m, 0.6H), 4.77-4.41 (m, 2.3H), 4.23-3.13 (m, 10.6H), 2.87-2.51 (m, 1.5H), 2.35-2.19 (m, 0.3H), 2.18-1.71 (m, 5.4H), 1.69-1.43 (m, 1.8H), 1.39-1.21 (m, 0.9H), 1.06-0.90 (m, 0.9H).

With this procedure, reaction of sodium hyaluronate (MW 2000 KDa) provided corresponding product (0.191 g, yield: 49.8%, DSR=39%). ¹H NMR (400 MHz, Deuterium Oxide) δ 7.70-7.54 (m, 0.39H), 6.58-6.47 (m, 0.39H), 6.39-6.25 (m, 0.39H), 5.43-5.30 (m, 0.39H), 5.19-5.00 (m, 0.78H), 4.78-4.44 (m, 2.39H), 4.25-3.15 (m, 10.78H), 2.89-2.50 (m, 1.95H), 2.34-2.23 (m, 0.39H), 2.21-1.72 (m, 6.12H), 1.70-1.46 (m, 2.34H), 1.41-1.23 (m, 1.17H), 1.06-0.91 (m, 1.17H).

### Example 3

### Preparation of Conjugate of HA and 4-aminobutyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

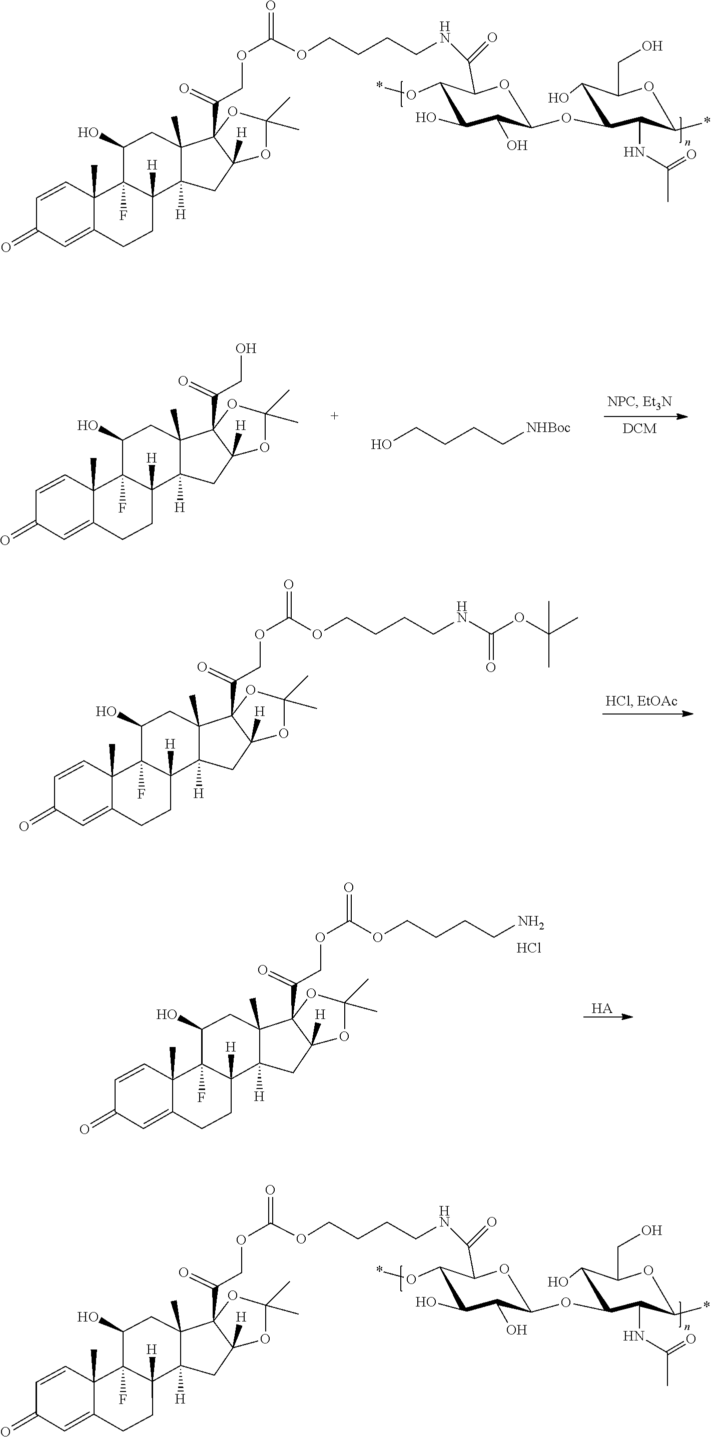

### Step 1: Preparation of tert-butyl (4-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12b-dodecahydro-8βH-naphtho[2′,1′:4,5]indeno[1,2-d][1,3]dioxol-8β-yl)-2-oxoethoxy)carbonyl)oxy)butyl)carbamate

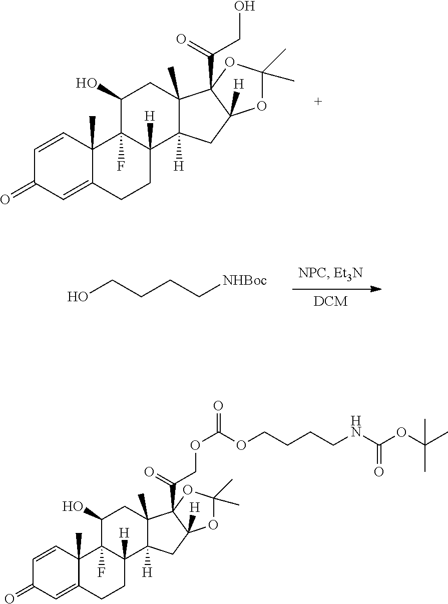

To a mixture of 9α-Fluoro-11β,16α,17,21-tetrahydroxy-1,4-pregnadiene-3,20-dione (Triamcinolone acetonide, 1304 mg, 3 mmol), and Bis (4-nitrophenyl) carbonate (1094 mg, 3.6 mmol) in dichloromethane (27 mL) was added triethylamine (1 mL, 7.2 mmol) under N₂, the reaction mixture was heated to reflux for 6 hours. Then tert-butyl (4-hydroxybutyl)carbamate (738 mg, 3.9 mmol) was added and the resulting mixture was stirred at room temperature for 16 hours. The solution was diluted with DCM and washed with saturated NaHCO₃ solution, water and saturated brine solution. The organic layer was dried over sodium sulfate, filtered and then concentrated under reduced pressure. The residue was purified by silical gel chromatography to give the title product (1.4 g, yield: 71.9%); MS (m/z): [M+H]⁺ calcd for C₃₄H₄₈FNO₁₀, 650.75; found, 594.3 (M+H−56).

### Step 2: Preparation of 4-aminobutyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride

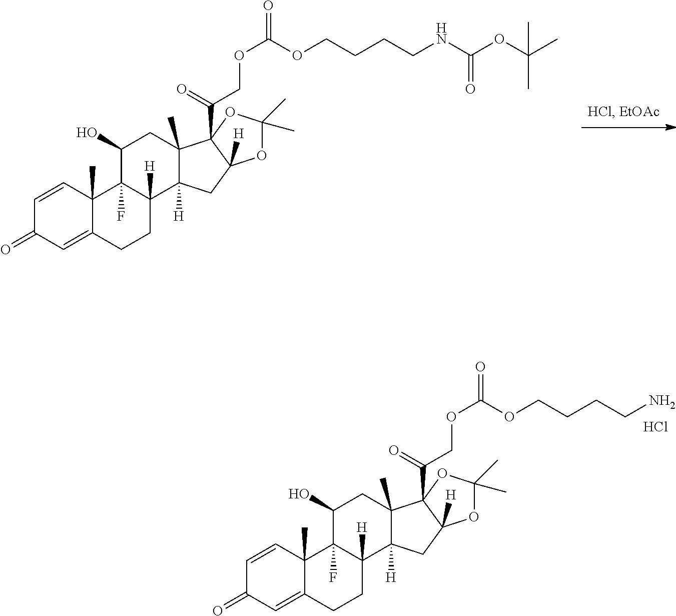

To a stirred solution of tert-butyl (4-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12b-dodecahydro-8βH-naphtho[2′,1′:4,5]indeno[1,2-d][1,3]dioxol-8β-yl)-2-oxoethoxy)carbonyl)oxy)butyl)carbamate (1000 mg, 1.54 mmol) in EtOAc (30 mL) was slowly added 4M HCl in ethyl acetate (6 mL) at ice-bath. The reaction mixture was allowed to stir at room temperature for 18 hours. The solution was diluted with EtOAc and concentrated under reduced pressure to afford the title compound as a white solid (0.9 g, yield: 99.8%) that was used without further purification. MS (m/z): [M+H]+ calcd for C₂₉H₄₀FNO₈, 550.64; found, 550.3.

### Step 3: Preparation of Conjugate of HA and 4-aminobutyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

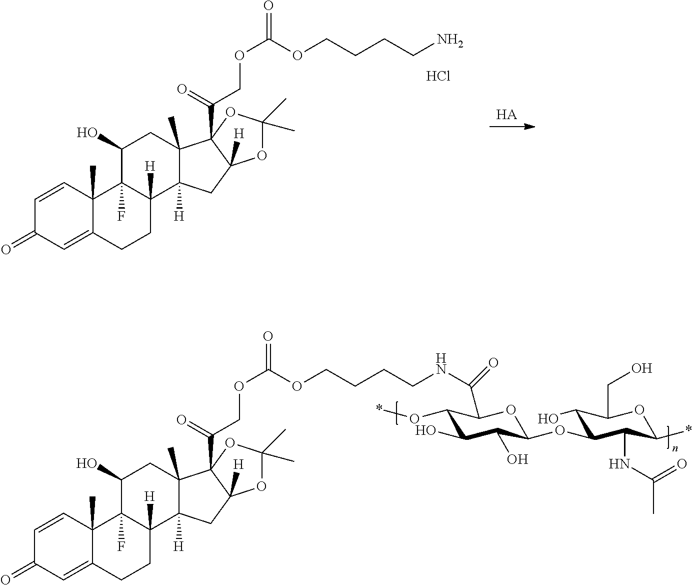

Hyaluronic acid (101 mg, 0.25 mmol carboxylic acid) was dissolved in 20 mL of deionized water in a 100 mL round-bottomed flask followed by the dropwise addition of 13 mL of acetonitrile while stirring. To the solution was added 4-methylmorpholine (NMM, 18 mg, 0.175 mmol) and 4-aminobutyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride (103 mg, 0.175 mmol) causing the viscosity to increase temporarily. The solution was then cooled to 0° C., and 4-(4,6-Dimethoxy-1,3,5-triazin-2-yl)-4-methylmorpholinium chloride (DMTMM, 49 mg, 0.175 mmol) was added and stirred at room temperature for 72 hours at room temperature. NaCl (147 mg, 2.5 mmol) was then added to the reaction mixture, which was stirred for 1 hour and followed by the dropwise addition of anhydrous alcohol (100 mL) while stirring. The mixture was filtered. The filter cake was collected, washed with anhydrous alcohol and dried in vacuo to give the title compound as a white solid. (sodium hyaluronate MW 50 KDa 0.096 g, yield: 48.7%, DSR=27%); ¹H NMR (400 MHz, Deuterium Oxide) δ 7.67-7.58 (m, 0.27H), 6.56-6.49 (m, 0.27H), 6.34-6.27 (m, 0.27H), 5.42-5.34 (m, 0.27H), 5.19-5.01 (m, 0.54H), 4.80-4.45 (m, 2.81H), 4.39-3.07 (m, 10.54H), 2.92-2.49 (m, 1.35H), 2.32-1.68 (m, 3.54H), 1.67-1.45 (m, 3.51H), 1.38-1.27 (m, 0.81H), 1.05-0.90 (m, 0.81H).

With this procedure, reaction of sodium hyaluronate (MW 500 KDa) provided corresponding product (0.068 g, yield: 34.5%, DSR=25%). ¹H NMR (400 MHz, Deuterium Oxide) δ 7.66-7.54 (m, 0.25H), 6.55-6.47 (m, 0.25H), 6.35-6.25 (m, 0.25H), 5.42-5.29 (m, 0.25H), 5.18-4.97 (m, 0.5H), 4.79-4.42 (m, 2.75H), 4.39-3.05 (m, 10.5H), 2.89-2.48 (m, 1.25H), 2.37-1.67 (m, 3.5H), 1.66-1.46 (m, 3.25H), 1.40-1.25 (m, 0.75H), 1.07-0.90 (m, 0.75H).

With this procedure, reaction of sodium hyaluronate (MW 2000 KDa) provided corresponding product (0.068 g, yield: 34.5%, DSR=15%). ¹H NMR (400 MHz, Deuterium Oxide) δ 7.66-7.54 (m, 0.15H), 6.57-6.43 (m, 0.15H), 6.34-6.25 (m, 0.15H), 5.43-5.29 (m, 0.15H), 5.18-4.97 (m, 0.3H), 4.79-4.43 (m, 2.45H), 4.40-3.01 (m, 10.3H), 2.86-2.50 (m, 0.75H), 2.37-1.66 (m, 3.3H), 1.65-1.46 (m, 1.95H), 1.39-1.24 (m, 0.45H), 1.07-0.91 (m, 0.45H).

### Example 4

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (4-aminobutyl)carbamate

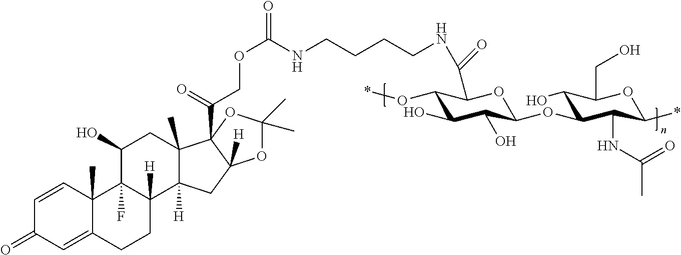

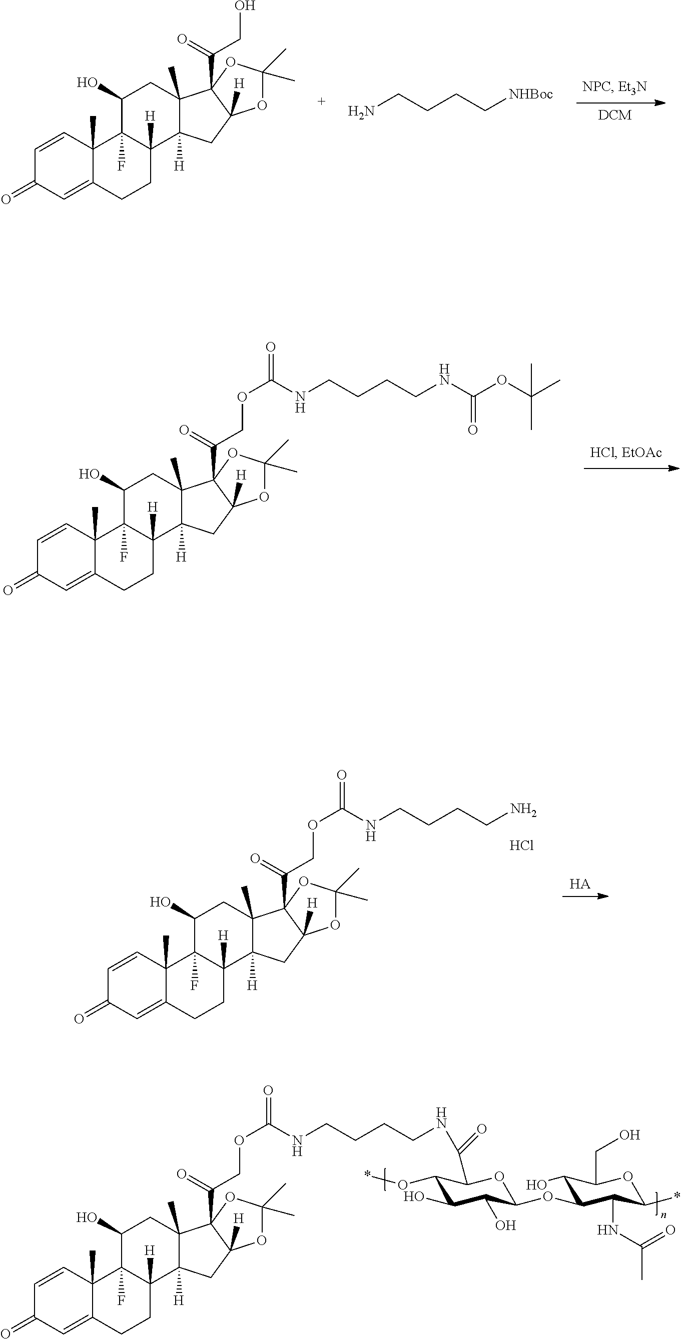

### Step 1: Preparation of tert-butyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) butane-1,4-diyldicarbamate

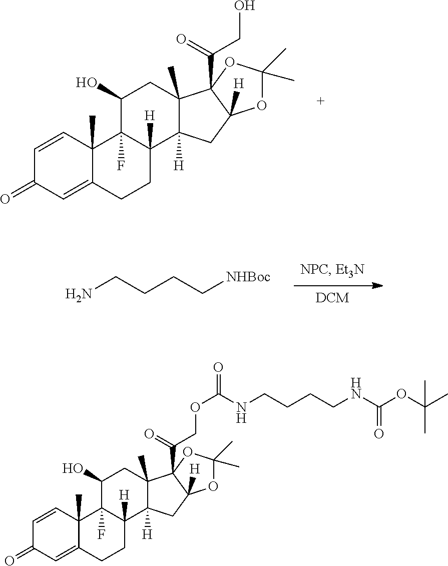

To a mixture of 9α-Fluoro-11β,16α,17,21-tetrahydroxy-1,4-pregnadiene-3,20-dione (Triamcinolone acetonide, 1090 mg, 2.5 mmol), and Bis (4-nitrophenyl) carbonate (912 mg, 3 mmol) in dichloromethane (80 mL) was added triethylamine (0.85 mL, 6 mmol) under N₂, the reaction mixture was heated to reflux for 6 hours. Then tert-butyl (4-aminobutyl) carbamate (612 mg, 3.25 mmol) was added and the resulting mixture was stirred at room temperature for 16 hours. The solution was diluted with DCM and washed with saturated NaHCO₃ solution, water and saturated brine solution. The organic layer was dried over sodium sulfate, filtered and then concentrated under reduced pressure. The residue was purified by silical gel chromatography to give the title product (1.24 g, yield: 76.5%); MS (m/z): [M+H]⁺ calcd for C₃₄H₄₉FN₂O₉, 649.77; found, 593.2 (M+H−56). ¹H NMR (400 MHz, Chloroform-d) δ 7.23 (d, J=10.1 Hz, 1H), 6.34 (dd, J=10.1, 1.9 Hz, 1H), 6.13 (t, J=1.7 Hz, 1H), 5.14 (t, J=5.9 Hz, 1H), 4.99 (d, J=4.8 Hz, 1H), 4.88 (t, J=13.2 Hz, 2H), 4.69-4.58 (m, 1H), 4.41 (dt, J=10.0, 2.5 Hz, 1H), 3.17 (dt, J=30.1, 6.1 Hz, 4H), 2.68-2.59 (m, 1H), 2.55-2.28 (m, 4H), 2.10 (d, J=4.8 Hz, 2H), 1.93-1.75 (m, 2H), 1.70-1.53 (m, 9H), 1.44 (d, J=6.8 Hz, 12H), 1.21 (s, 3H), 0.96 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (4-aminobutyl)carbamate hydrochloride

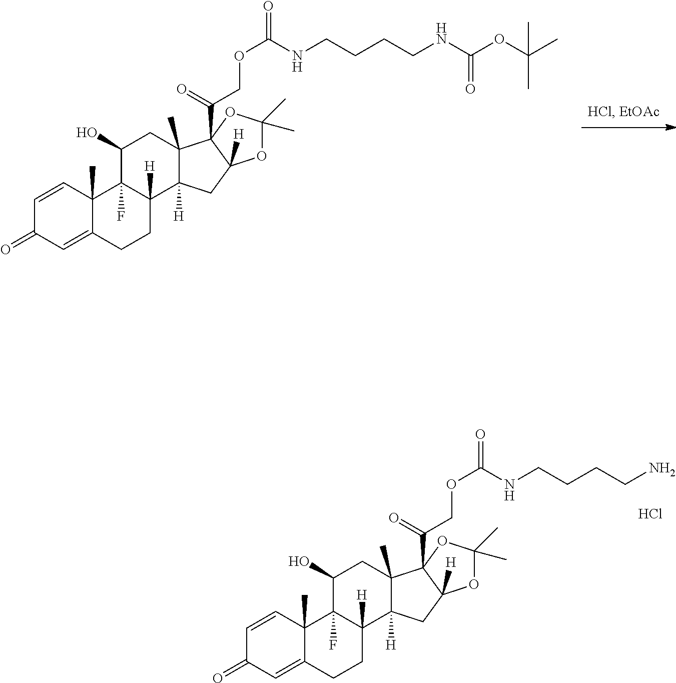

To a stirred solution of tert-butyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) butane-1,4-diyldicarbamate (400 mg, 0.62 mmol) in EtOAc (8 mL) was slowly added 4M HCl in ethyl acetate (1.6 mL) at ice-bath. The reaction mixture was allowed to stir at room temperature for 18 hours. The solution was diluted with EtOAc and concentrated under reduced pressure to afford the title compound as a white solid (0.3 g, yield: 82.8%) that was used without further purification. MS (m/z): [M+H]+ calcd for C₂₉H₄₁FN₂O₇, 549.65; found, 549.3.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (4-aminobutyl)carbamate

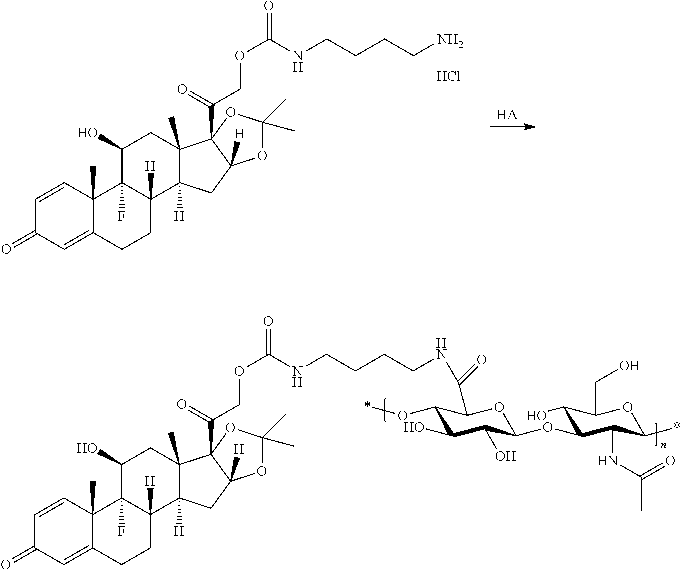

Hyaluronic acid (161 mg, 0.4 mmol carboxylic acid) was dissolved in 30 mL of deionized water in a 100 mL round-bottomed flask followed by the dropwise addition of 21 mL of acetonitrile while stirring. To the solution was added 4-methylmorpholine (NMM, 35 mg, 0.35 mmol) and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (4-aminobutyl)carbamate hydrochloride (164 mg, 0.28 mmol) causing the viscosity to increase temporarily. The solution was then cooled to 0° C., and 4-(4,6-Dimethoxy-1,3,5-triazin-2-yl)-4-methylmorpholinium chloride (DMTMM, 78 mg, 0.28 mmol) was added and stirred at room temperature for 72 hours at room temperature. NaCl (234 mg, 4 mmol) was then added to the reaction mixture, which was stirred for 1 hour and followed by the dropwise addition of anhydrous alcohol (200 mL) while stirring. The mixture was filtered. The filter cake was collected, washed with anhydrous alcohol and dried in vacuo to give the title compound as a white solid. (sodium hyaluronate MW 50 KDa 0.151 g, yield: 48%, DSR=27%); ¹H NMR (400 MHz, Deuterium Oxide) δ 7.69-7.56 (m, 0.27H), 6.59-6.46 (m, 0.27H), 6.36-6.25 (m, 0.27H), 5.36-5.20 (m, 0.27H), 5.19-4.90 (m, 0.54H), 4.76-4.42 (m, 2.27H), 4.26-3.13 (m, 11.08H), 2.92-2.48 (m, 1.08H), 2.39-2.22 (m, 0.54H), 2.21-1.98 (m, 3.54H), 1.95-1.48 (m, 3.24H), 1.42-1.26 (m, 0.81H), 1.10-0.92 (m, 0.81H).

With this procedure, reaction of sodium hyaluronate (MW 500 KDa) provided corresponding product (0.183 g, yield: 58.1%, DSR=25%). ¹H NMR (400 MHz, Deuterium Oxide) δ 7.69-7.56 (m, 0.25H), 6.59-6.45 (m, 0.25H), 6.37-6.23 (m, 0.25H), 5.37-5.05 (m, 0.75H), 4.76-4.41 (m, 2.25H), 4.25-3.09 (m, 11H), 2.91-2.46 (m, 1H), 2.34-2.20 (m, 0.5H), 2.19-1.98 (m, 3.5H), 1.94-1.46 (m, 3H), 1.41-1.27 (m, 0.75H), 1.09-0.92 (m, 0.75H).

With this procedure, reaction of sodium hyaluronate (MW 2000 KDa) provided corresponding product (0.142 g, yield: 45.1%, DSR=15%). ¹H NMR (400 MHz, Deuterium Oxide) δ 7.69-7.58 (m, 0.15H), 6.60-6.49 (m, 0.15H), 6.40-6.26 (m, 0.15H), 5.39-5.00 (m, 0.45H), 4.80-4.47 (m, 2.15H), 4.20-3.12 (m, 10.6H), 2.92-2.47 (m, 0.6H), 2.40-2.23 (m, 0.3H), 2.21-1.97 (m, 3.3H), 1.95-1.46 (m, 1.8H), 1.41-1.25 (m, 0.45H), 1.08-0.93 (m, 0.45H).

### Example 5

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-aminopropanoate

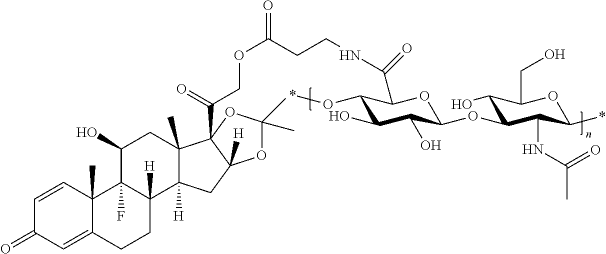

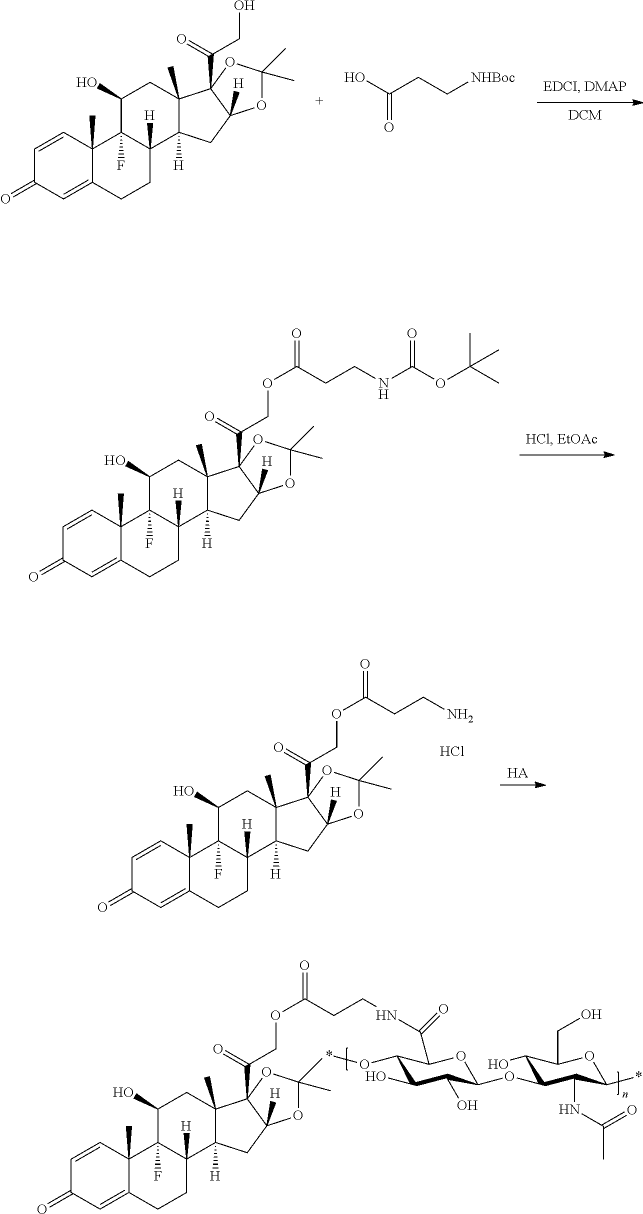

### Step 1: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-((tert-butoxycarbonyl)amino)propanoate

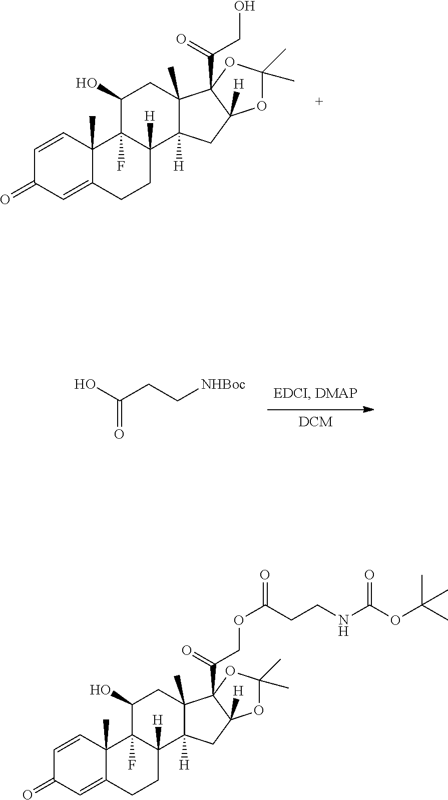

9α-Fluoro-11β,16α,17,21-tetrahydroxy-1,4-pregnadiene-3,20-dione (Triamcinolone acetonide, 679 mg, 1.56 mmol), 3-((tert-butoxycarbonyl)amino)propanoic acid (387.5 mg, 1.8 mmol), 1-ethyl-3-(3-dimethylaminopropyl)carbodiimide (EDCI, 432 mg, 2.25 mmol), N,N-dimethylpyridin-4-amine (DMAP, 18 mg, 0.15 mmol) were dissolved in DCM (20 mL). The reaction mixture was stirred at room temperature for 24 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=5:1˜2:1) to afford the title compound (650 mg, Yield: 68.8%). MS (m/z): [M+H]⁺ calcd for C₃₂H₄₄FNO₉, 606.70; found, 606.2. ¹H NMR (400 MHz, Chloroform-d) δ 7.22 (d, J=10.1 Hz, 1H), 6.35 (dd, J=10.2, 1.9 Hz, 1H), 6.14 (t, J=1.7 Hz, 1H), 5.17 (s, 1H), 4.99 (dd, J=11.3, 6.4 Hz, 2H), 4.88 (d, J=17.6 Hz, 1H), 4.42 (dt, J=8.4, 2.8 Hz, 1H), 3.46 (t, J=6.4 Hz, 2H), 2.70-2.58 (m, 3H), 2.53-2.29 (m, 4H), 2.11 (td, J=12.6, 5.9 Hz, 1H), 1.87 (dt, J=11.9, 5.4 Hz, 1H), 1.73-1.60 (m, 4H), 1.58 (d, J=1.5 Hz, 3H), 1.44 (d, J=4.0 Hz, 12H), 1.22 (s, 3H), 0.94 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-aminopropanoate hydrochloride

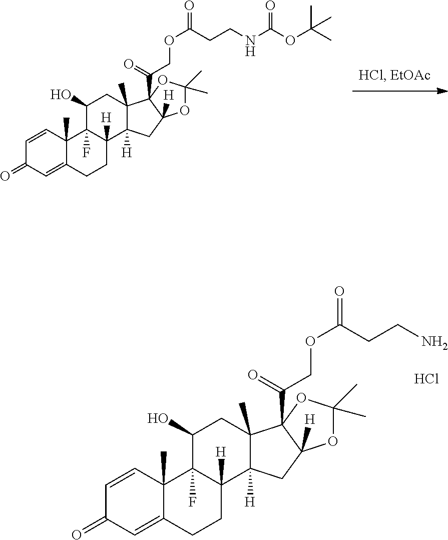

To a stirred solution of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-((tert-butoxycarbonyl)amino)propanoate (605 mg, 1 mmol) in EtOAc (25 mL) was slowly added 4M HCl in ethyl acetate (5 mL) at ice-bath. The reaction mixture was allowed to stir at room temperature for 40 hours. The solution was diluted with EtOAc and concentrated under reduced pressure to afford the title compound as a white solid (0.54 g, yield: 99.7%) that was used without further purification. MS (m/z): [M+H]+ calcd for C₂₇H₃₆FNO₇, 506.58; found, 506.1.

### Step 3: Preparation of Conjugate of HA (Hyaluronic acid) and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-aminopropanoate

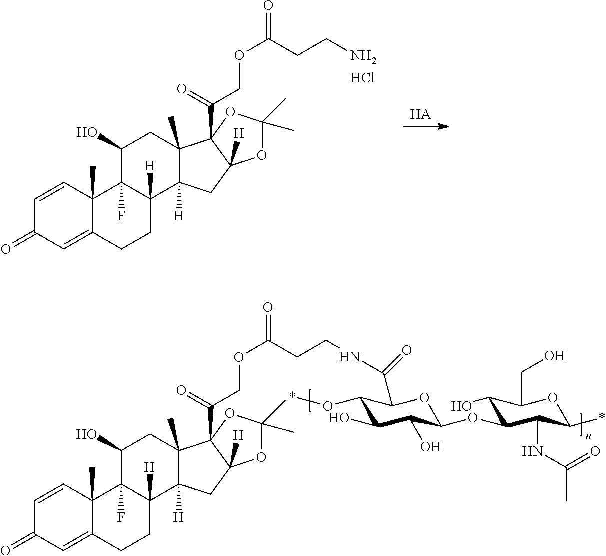

Hyaluronic acid (161 mg, 0.4 mmol carboxylic acid) was dissolved in 30 mL of deionized water in a 100 mL round-bottomed flask followed by the dropwise addition of 21 mL of acetonitrile while stirring. To the solution was added 4-methylmorpholine (NMM, 28 mg, 0.28 mmol) and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-aminopropanoate hydrochloride (152 mg, 0.28 mmol) causing the viscosity to increase temporarily. The solution was then cooled to 0° C., and 4-(4,6-Dimethoxy-1,3,5-triazin-2-yl)-4-methylmorpholinium chloride (DMTMM, 78 mg, 0.28 mmol) was added and stirred at room temperature for 72 hours at room temperature. NaCl (234 mg, 4 mmol) was then added to the reaction mixture, which was stirred for 1 hour and followed by the dropwise addition of anhydrous alcohol (200 mL) while stirring. The mixture was filtered. The filter cake was collected, washed with anhydrous alcohol and dried in vacuo to give the title compound as a white solid. (sodium hyaluronate MW 50 KDa 0.194 g, yield: 64.1%, DSR=26%); ¹H NMR (400 MHz, Deuterium Oxide) δ 7.67-7.51 (m, 0.26H), 6.55-6.39 (m, 0.26H), 6.33-6.19 (m, 0.26H), 5.40-5.23 (m, 0.26H), 5.16-4.94 (m, 0.52H), 4.76-4.32 (m, 2.26H), 4.18-3.12 (m, 10.52H), 2.99-2.39 (m, 1.56H), 2.30-2.17 (m, 0.26H), 2.16-1.67 (m, 4.3H), 1.65-1.42 (m, 1.56H), 1.35-1.18 (m, 0.78H), 1.04-0.86 (m, 0.78H).

With this procedure, reaction of sodium hyaluronate (MW 500 KDa) provided corresponding product (0.23 g, yield: 76%, DSR=22%). ¹H NMR (400 MHz, Deuterium Oxide) δ 7.66-7.52 (m, 0.22H), 6.55-6.39 (m, 0.22H), 6.32-6.18 (m, 0.22H), 5.36-5.23 (m, 0.22H), 5.16-4.95 (m, 0.44H), 4.77-4.32 (m, 2.22H), 4.19-3.09 (m, 10.44H), 2.94-2.40 (m, 1.32H), 2.29-2.18 (m, 0.22H), 2.16-1.91 (m, 4.1H), 1.90-1.42 (m, 1.32H), 1.37-1.18 (m, 0.66H), 1.02-0.85 (m, 0.66H).

With this procedure, reaction of sodium hyaluronate (MW 2000 KDa) provided corresponding product (0.099 g, yield: 32.7%, DSR=30%). ¹H NMR (400 MHz, Deuterium Oxide) δ 7.66-7.51 (m, 0.3H), 6.55-6.40 (m, 0.3H), 6.32-6.21 (m, 0.3H), 5.39-5.25 (m, 0.3H), 5.14-4.94 (m, 0.6H), 4.76-4.35 (m, 2.3H), 4.21-3.14 (m, 10.6H), 2.95-2.45 (m, 1.8H), 2.38-2.22 (m, 0.3H), 2.18-1.68 (m, 4.5H), 1.65-1.43 (m, 1.8H), 1.38-1.20 (m, 0.9H), 1.05-0.87 (m, 0.9H).

### Example 6

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl pyrrolidine-3-carboxylate

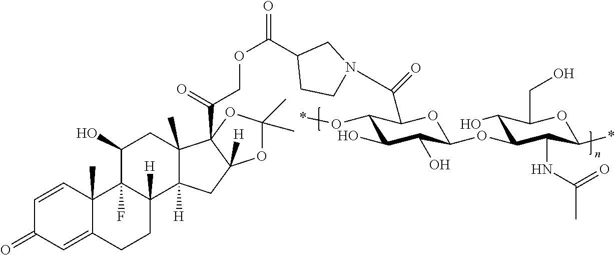

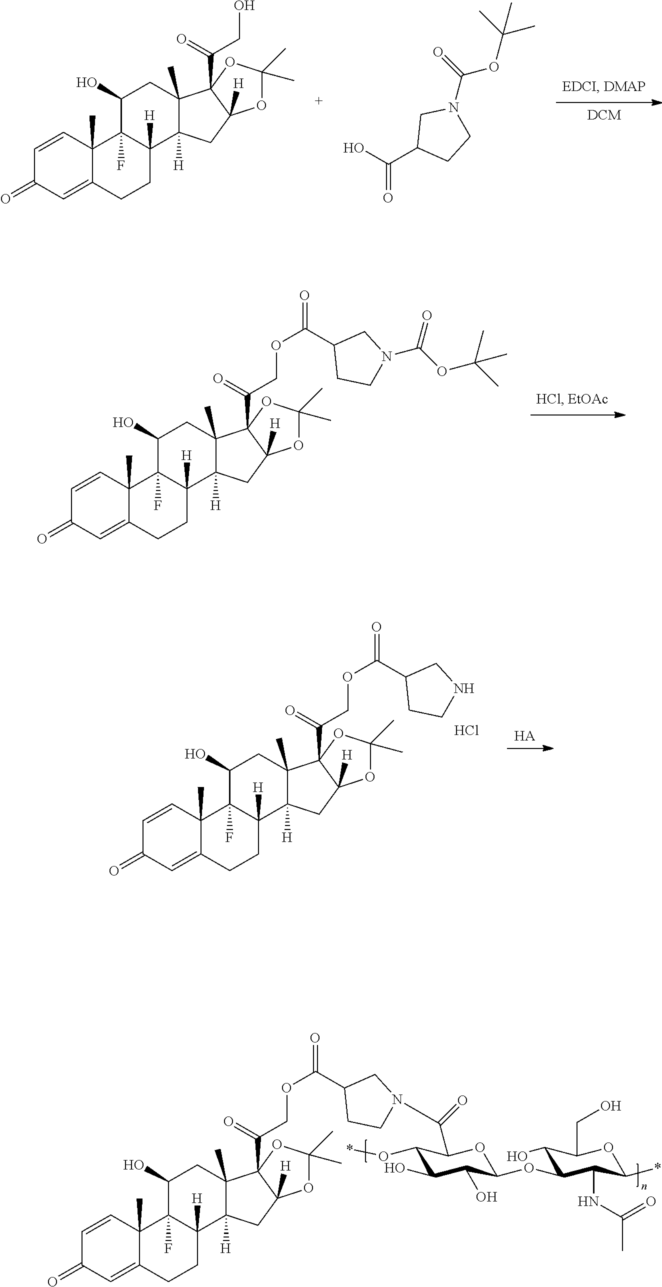

### Step 1: Preparation of 1-(tert-butyl) 3-(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) pyrrolidine-1,3-dicarboxylate

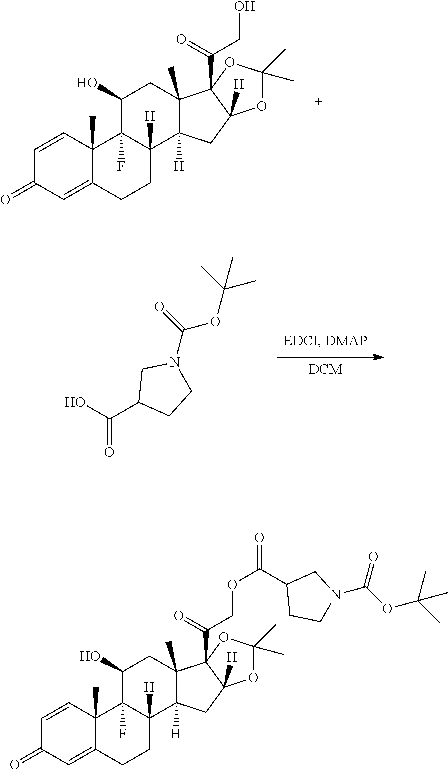

9α-Fluoro-11β, 16α, 17,21-tetrahydroxy-1,4-pregnadiene-3,20-dione (Triamcinolone acetonide, 652 mg, 1.5 mmol), 1-(tert-butoxycarbonyl)pyrrolidine-3-carboxylic acid (420 mg, 1.95 mmol), 1-ethyl-3-(3-dimethylaminopropyl)carbodiimide (EDCI, 375 mg, 1.95 mmol), N,N-dimethylpyridin-4-amine (DMAP, 18 mg, 0.15 mmol) were dissolved in DCM (20 mL). The reaction mixture was stirred at room temperature for 3 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=5:1˜2:1) to afford the title compound (910 mg, Yield: 96.1%). MS (m/z): [M+H]⁺ calcd for C₃₄H₄₆FNO₉, 632.74; found, 632.2. ¹H NMR (400 MHz, Chloroform-d) δ 7.22 (d, J=10.1 Hz, 1H), 6.35 (dd, J=10.1, 1.9 Hz, 1H), 6.13 (t, J=1.7 Hz, 1H), 5.05-4.80 (m, 3H), 4.42 (s, 1H), 3.70-3.33 (m, 4H), 3.19 (q, J=7.2, 6.8 Hz, 1H), 2.69-2.44 (m, 2H), 2.39 (dd, J=12.9, 5.4 Hz, 2H), 2.30-1.99 (m, 4H), 1.91-1.82 (m, 1H), 1.70-1.58 (m, 4H), 1.55 (s, 3H), 1.46 (s, 9H), 1.43 (s, 3H), 1.22 (s, 3H), 0.93 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl pyrrolidine-3-carboxylate hydrochloride

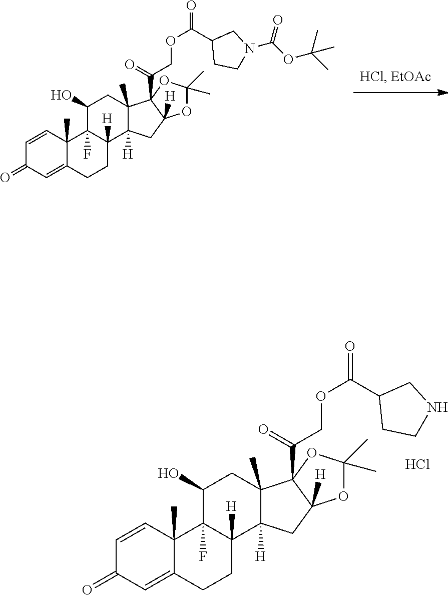

To a stirred solution of 1-(tert-butyl) 3-(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) pyrrolidine-1,3-dicarboxylate (600 mg, 0.95 mmol) in EtOAc (24 mL) was slowly added 4M HCl in ethyl acetate (4.8 mL) at ice-bath. The reaction mixture was allowed to stir at room temperature for 18 hours. The solution was diluted with EtOAc and concentrated under reduced pressure to afford the title compound as a white solid (0.53 g, yield: 97.6%) that was used without further purification. MS (m/z): [M+H]+ calcd for C₂₉H₃₈FNO₇, 532.62; found, 532.3.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl pyrrolidine-3-carboxylate

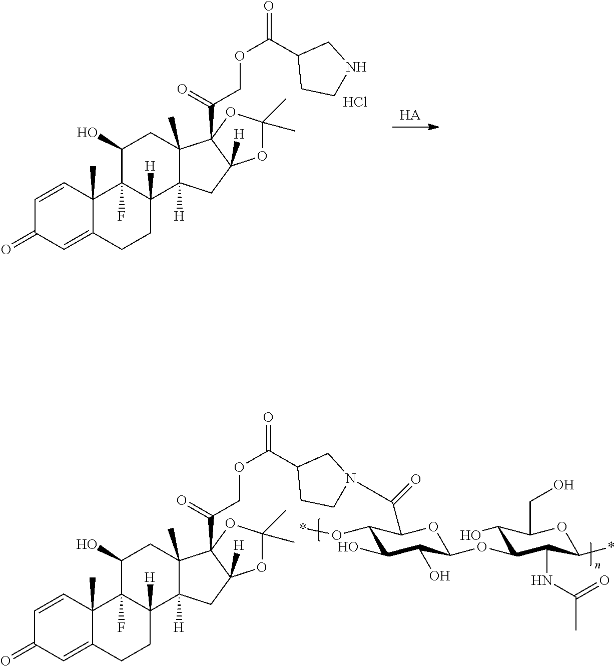

Hyaluronic acid (201 mg, 0.5 mmol carboxylic acid) was dissolved in 40 mL of deionized water in a 100 mL round-bottomed flask followed by the dropwise addition of 20 mL of acetonitrile while stirring. To the solution was added 4-methylmorpholine (NMM, 35 mg, 0.35 mmol) and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl pyrrolidine-3-carboxylate hydrochloride (199 mg, 0.35 mmol) causing the viscosity to increase temporarily. The solution was then cooled to 0° C., and 4-(4,6-Dimethoxy-1,3,5-triazin-2-yl)-4-methylmorpholinium chloride (DMTMM, 97 mg, 0.35 mmol) was added and stirred at room temperature for 72 hours at room temperature. NaCl (439 mg, 7.5 mmol) was then added to the reaction mixture, which was stirred for 1 hour and followed by the dropwise addition of anhydrous alcohol (200 mL) while stirring. The mixture was filtered. The filter cake was collected, washed with anhydrous alcohol and dried under vacuo to give the title compound as a white solid. (sodium hyaluronate MW 50 KDa 0.199 g, yield: 51.4%, DSR=22%); ¹H NMR (400 MHz, Deuterium Oxide) δ 7.68-7.48 (m, 0.22H), 6.57-6.38 (m, 0.22H), 6.33-6.17 (m, 0.22H), 5.47-5.23 (m, 0.22H), 5.17-4.98 (m, 0.44H), 4.77-4.23 (m, 2.22H), 4.19-2.99 (m, 11.1H), 2.87-2.38 (m, 0.88H), 2.32-1.68 (m, 4.76H), 1.66-1.42 (m, 1.32H), 1.38-1.16 (m, 0.66H), 1.03-0.82 (m, 0.66H).

With this procedure, reaction of sodium hyaluronate (MW 500 KDa) provided corresponding product (0.235 g, yield: 60.6%, DSR=7%). ¹H NMR (400 MHz, Deuterium Oxide) δ 7.71-7.62 (m, 0.07H), 6.61-6.53 (m, 0.07H), 6.42-6.33 (m, 0.07H), 5.47-5.37 (m, 0.07H), 5.28-5.00 (m, 0.14H), 4.77-4.43 (m, 2.07H), 4.26-3.02 (m, 10.35H), 2.91-2.58 (m, 0.28H), 2.42-1.77 (m, 3.56H), 1.74-1.58 (m, 0.42H), 1.44-1.35 (m, 0.21H), 1.08-0.95 (m, 0.21H).

With this procedure, reaction of sodium hyaluronate (MW 2000 KDa) provided corresponding product (0.188 g, yield: 48.5%, DSR=5%). ¹H NMR (400 MHz, Deuterium Oxide) δ 7.72-7.62 (m, 0.05H), 6.62-6.52 (m, 0.05H), 6.41-6.33 (m, 0.05H), 5.48-5.38 (m, 0.05H), 5.26-5.01 (m, 0.1H), 4.80-4.40 (m, 2.05H), 4.33-2.98 (m, 10.25H), 2.89-2.55 (m, 0.2H), 2.42-1.75 (m, 3.4H), 1.73-1.55 (m, 0.3H), 1.44-1.33 (m, 0.15H), 1.10-0.97 (m, 0.15H).

### Example 7

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl azetidine-3-carboxylate

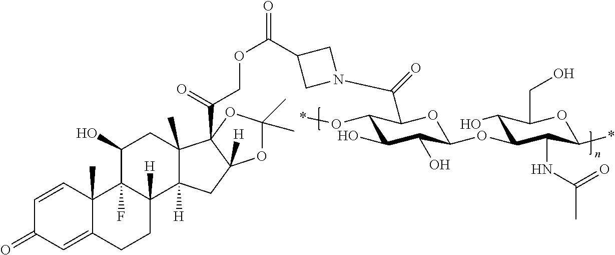

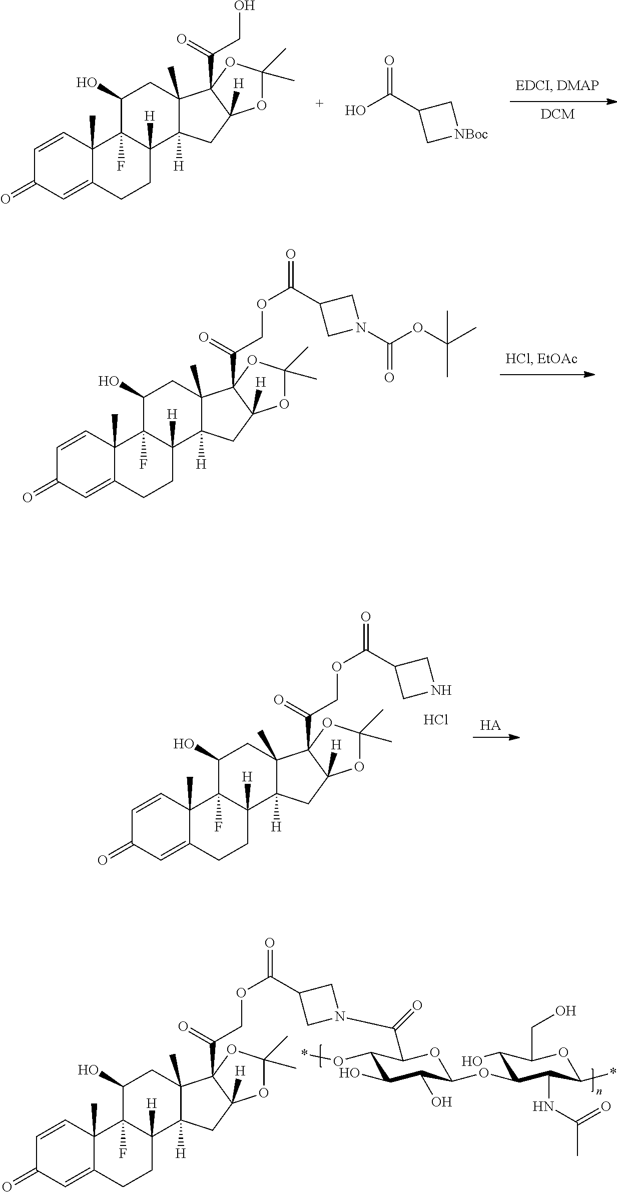

### Step 1: Preparation of 1-(tert-butyl) 3-(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) azetidine-1,3-dicarboxylate

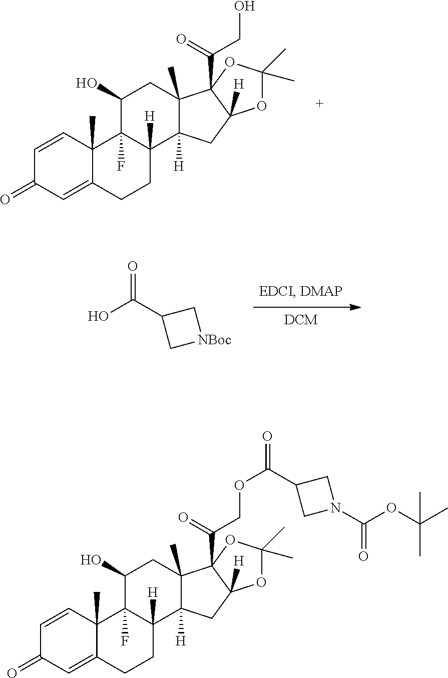

9α-Fluoro-11β,16α,17,21-tetrahydroxy-1,4-pregnadiene-3,20-dione (Triamcinolone acetonide, 434.5 mg, 1 mmol), 1-(tert-butoxycarbonyl)azetidine-3-carboxylic acid (262 mg, 1.3 mmol), 1-ethyl-3-(3-dimethylaminopropyl)carbodiimide (EDCI, 249 mg, 1.3 mmol), N,N-dimethylpyridin-4-amine (DMAP, 13 mg, 0.1 mmol) were dissolved in DCM (20 mL). The reaction mixture was stirred at room temperature for 2 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=5:1˜2:1) to afford the title compound (600 mg, Yield: 97.2%). MS (m/z): [M+H]⁺ calcd for C₃₃H₄₄FNO₉, 618.71; found, 618.2. ¹H NMR (400 MHz, Chloroform-d) δ 7.22 (d, J=10.1 Hz, 1H), 6.36 (dd, J=10.2, 1.9 Hz, 1H), 6.14 (t, J=1.7 Hz, 1H), 5.10-4.96 (m, 2H), 4.90 (d, J=17.6 Hz, 1H), 4.43 (q, J=3.4 Hz, 1H), 4.24-4.09 (m, 4H), 3.48 (tt, J=8.5, 6.4 Hz, 1H), 2.70-2.57 (m, 1H), 2.55-2.27 (m, 4H), 2.11 (td, J=12.5, 5.9 Hz, 1H), 1.87 (dt, J=11.6, 5.1 Hz, 1H), 1.77-1.59 (m, 4H), 1.58 (s, 3H), 1.44 (d, J=2.6 Hz, 12H), 1.22 (s, 3H), 0.95 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12αa,12β-dodecahydro-8βH-naphtho[2′,1′:4,5]indeno[1,2-d][1,3]dioxol-8β-yl)-2-oxoethyl azetidine-3-carboxylate hydrochloride

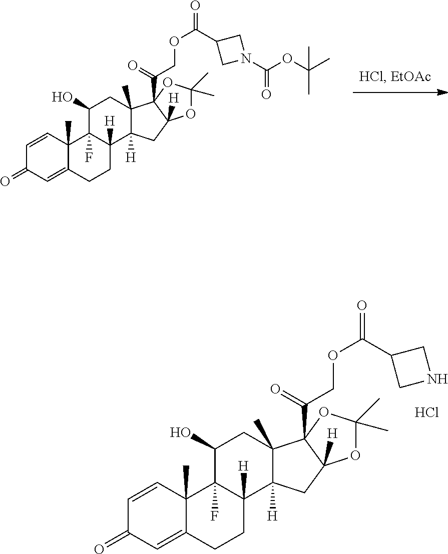

To a stirred solution of 1-(tert-butyl) 3-(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) azetidine-1,3-dicarboxylate (300 mg, 0.49 mmol) in EtOAc (12 mL) was slowly added 4M HCl in ethyl acetate (2.4 mL) at ice-bath. The reaction mixture was allowed stir at room temperature for 18 hours. The solution was diluted with EtOAc and concentrated under reduced pressure to afford the title compound as a white solid (0.25 g, yield: 92.9%) that was used without further purification. MS (m/z): [M+H]+ calcd for C₂₈H₃₆FNO₇, 518.59; found, 518.3.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl azetidine-3-carboxylate

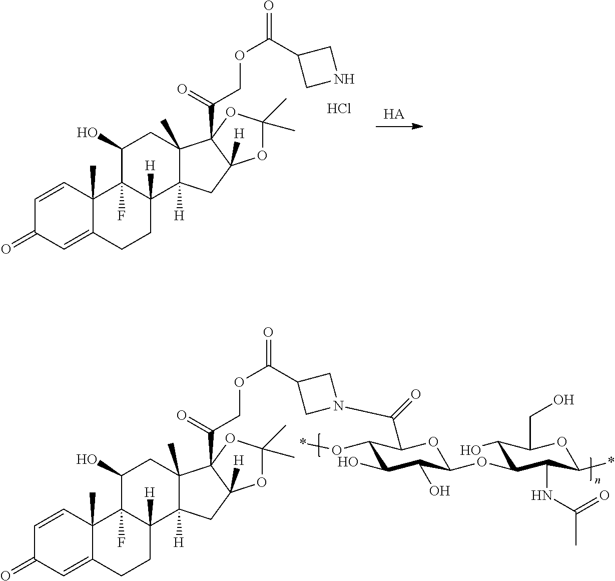

Hyaluronic acid (161 mg, 0.4 mmol carboxylic acid) was dissolved in 30 mL of deionized water in a 100 mL round-bottomed flask followed by the dropwise addition of 21 mL of acetonitrile while stirring. To the solution was added 4-methylmorpholine (NMM, 28 mg, 0.28 mmol) and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl azetidine-3-carboxylate hydrochloride (155 mg, 0.28 mmol) causing the viscosity to increase temporarily. The solution was then cooled to 0° C., and 4-(4,6-Dimethoxy-1,3,5-triazin-2-yl)-4-methylmorpholinium chloride (DMTMM, 78 mg, 0.28 mmol) was added and stirred at room temperature for 72 hours at room temperature. NaCl (234 mg, 4 mmol) was then added to the reaction mixture, which was stirred for 1 hour and followed by the dropwise addition of anhydrous alcohol (200 mL) while stirring. The mixture was filtered. The filter cake was collected, washed with anhydrous alcohol and dried under vacuo to give the title compound as a white solid. (sodium hyaluronate MW 50 KDa 0.24 g, yield: 78.4%, DSR=15%); ¹H NMR (400 MHz, Deuterium Oxide) δ 7.75-7.61 (m, 0.15H), 6.65-6.53 (m, 0.15H), 6.43-6.32 (m, 0.15H), 5.58-5.44 (m, 0.15H), 5.26-5.11 (m, 0.3H), 4.81-4.41 (m, 2.15H), 4.34-3.38 (m, 10.75H), 2.99-2.57 (m, 0.3H), 2.45-1.80 (m, 4.2H), 1.77-1.52 (m, 0.9H), 1.48-1.37 (m, 0.45H), 1.14-1.00 (m, 0.45H).

With this procedure, reaction of sodium hyaluronate (MW 500 KDa) provided corresponding product (0.236 g, yield: 77%, DSR=19%). ¹H NMR (400 MHz, Deuterium Oxide) δ 7.73-7.62 (m, 0.19H), 6.64-6.52 (m, 0.19H), 6.43-6.31 (m, 0.19H), 5.58-5.42 (m, 0.19H), 5.27-5.12 (m, 0.38H), 4.81-4.40 (m, 2.19H), 4.30-3.19 (m, 10.95H), 2.95-2.55 (m, 0.38H), 2.41-1.75 (m, 4.52H), 1.74-1.53 (m, 1.14H), 1.47-1.35 (m, 0.57H), 1.12-0.97 (m, 0.57H).

With this procedure, reaction of sodium hyaluronate (MW 2000 KDa) provided corresponding product (0.166 g, yield: 54.2%, DSR=20%). ¹H NMR (400 MHz, Deuterium Oxide) δ 7.76-7.61 (m, 0.2H), 6.64-6.52 (m, 0.2H), 6.43-6.32 (m, 0.2H), 5.56-5.44 (m, 0.2H), 5.27-5.09 (m, 0.4H), 4.80-4.43 (m, 2.2H), 4.26-3.11 (m, 11H), 2.96-2.50 (m, 0.4H), 2.37-1.75 (m, 4.6H), 1.74-1.50 (m, 1.2H), 1.45-1.34 (m, 0.6H), 1.11-0.95 (m, 0.6H).

### Example 8

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl alaninate

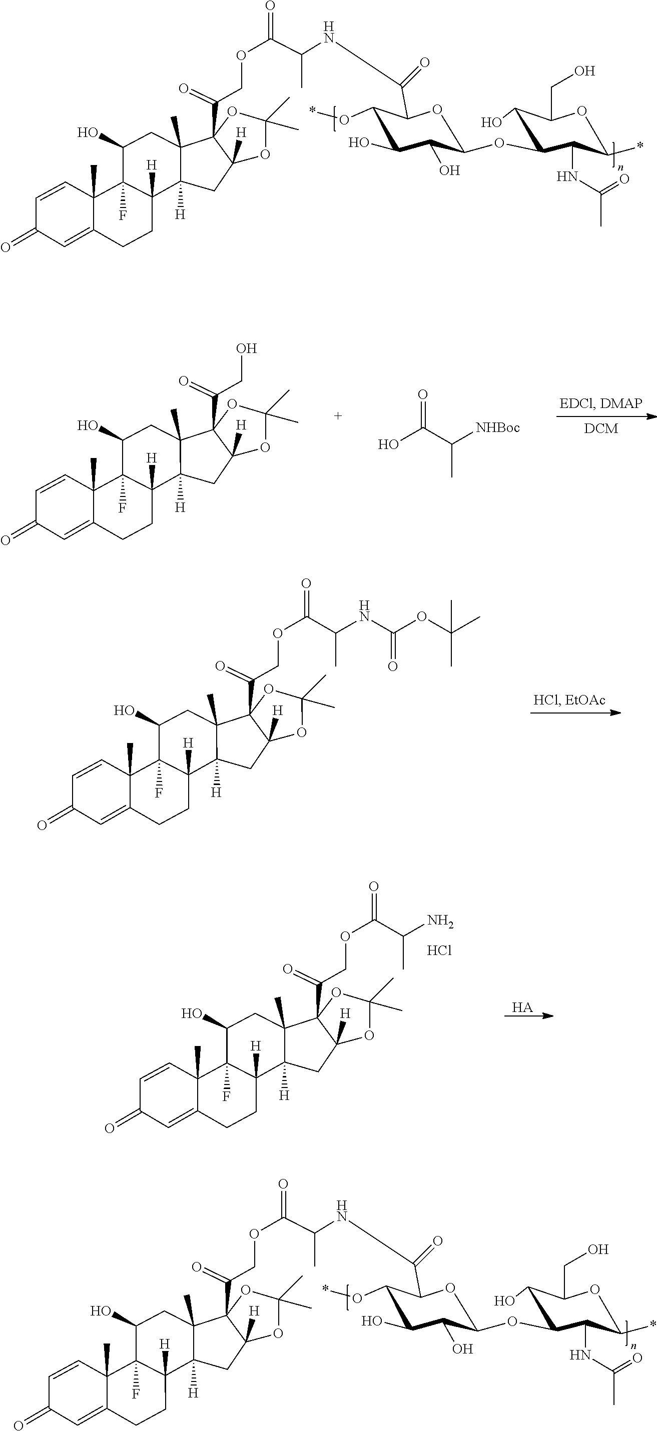

### Step 1: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (tert-butoxycarbonyl)alaninate

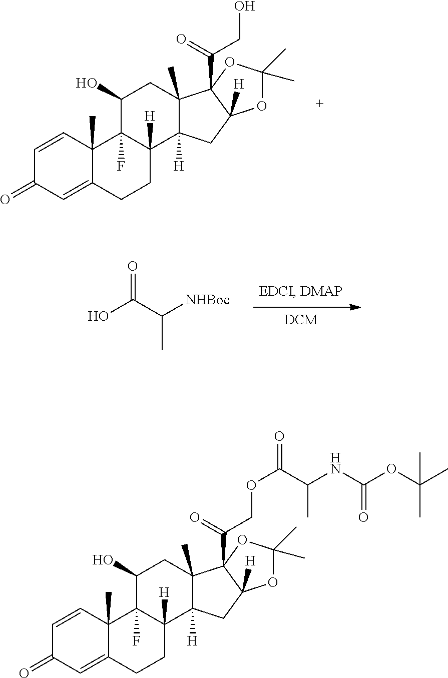

9α-Fluoro-11β,16α,17,21-tetrahydroxy-1,4-pregnadiene-3,20-dione (Triamcinolone acetonide, 652 mg, 1.5 mmol), (tert-butoxycarbonyl)alanine (369 mg, 1.95 mmol), 1-ethyl-3-(3-dimethylaminopropyl)carbodiimide (EDCI, 374 mg, 1.95 mmol), N,N-dimethylpyridin-4-amine (DMAP, 18 mg, 0.15 mmol) were dissolved in DCM (30 mL). The reaction mixture was stirred at room temperature for 2 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=5:1˜2:1) to afford the title compound (900 mg, Yield: 99.1%). MS (m/z): [M+H]⁺ calcd for C₃₂H₄₄FNO₉, 606.70; found, 606.2. ¹H NMR (400 MHz, Chloroform-d) δ 7.22 (dd, J=10.1, 2.1 Hz, 1H), 6.35 (dd, J=10.2, 1.9 Hz, 1H), 6.14 (t, J=1.7 Hz, 1H), 5.13-4.78 (m, 4H), 4.42 (d, J=8.4 Hz, 2H), 2.72-2.56 (m, 1H), 2.57-2.21 (m, 4H), 2.10 (td, J=12.5, 5.8 Hz, 1H), 1.92-1.81 (m, 1H), 1.79-1.58 (m, 4H), 1.55 (m, 3H), 1.51 (dd, J=11.0, 7.4 Hz, 3H), 1.44 (d, J=6.6 Hz, 12H), 1.22 (d, J=3.2 Hz, 3H), 0.93 (d, J=1.9 Hz, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl alaninate hydrochloride

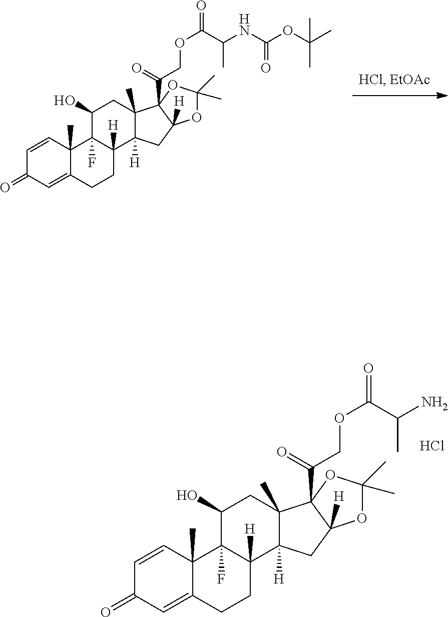

To a stirred solution of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (tert-butoxycarbonyl)alaninate (600 mg, 0.99 mmol) in EtOAc (12 mL) was slowly added 4M HCl in ethyl acetate (2.4 mL) at ice-bath. The reaction mixture was allowed to stir at room temperature for 18 hours. The solution was diluted with EtOAc and concentrated under reduced pressure to afford the title compound as a white solid (0.51 g, yield: 95.1%) that was used without further purification. MS (m/z): [M+H]+ calcd for C₂₇H₃₆FNO₇, 506.58; found, 506.4.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl alaninate

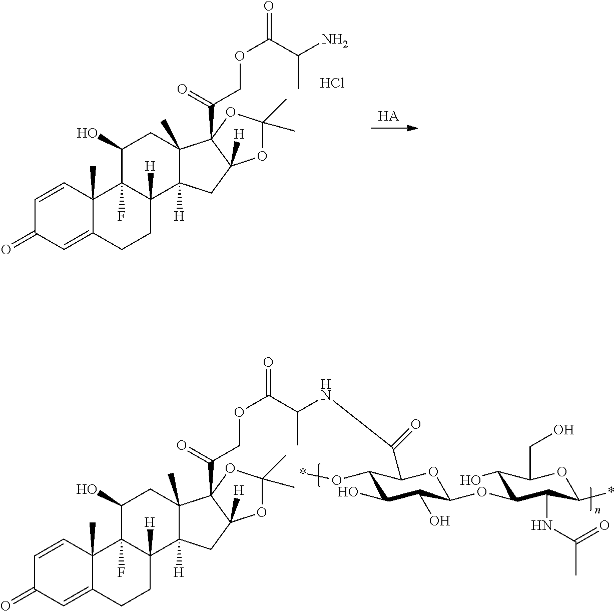

Hyaluronic acid (161 mg, 0.4 mmol carboxylic acid) was dissolved in 30 mL of deionized water in a 100 mL round-bottomed flask followed by the dropwise addition of 21 mL of acetonitrile while stirring. To the solution was added 4-methylmorpholine (NMM, 28 mg, 0.28 mmol) and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl alaninate hydrochloride (155 mg, 0.28 mmol) causing the viscosity to increase temporarily. The solution was then cooled to 0° C., and 4-(4,6-Dimethoxy-1,3,5-triazin-2-yl)-4-methylmorpholinium chloride (DMTMM, 78 mg, 0.28 mmol) was added and stirred at room temperature for 72 hours at room temperature. NaCl (234 mg, 4 mmol) was then added to the reaction mixture, which was stirred for 1 hour and followed by the dropwise addition of anhydrous alcohol (200 mL) while stirring. The mixture was filtered. The filter cake was collected, washed with anhydrous alcohol and dried in vacuo to give the title compound as a white solid. (sodium hyaluronate MW 50 KDa 0.208 g, yield: 68.7%, DSR=20%); ¹H NMR (400 MHz, Deuterium Oxide) δ 7.74-7.60 (m, 0.2H), 6.64-6.53 (m, 0.2H), 6.43-6.31 (m, 0.2H), 5.57-5.41 (m, 0.2H), 5.22-5.05 (m, 0.4H), 4.85-4.47 (m, 2.4H), 4.24-3.36 (m, 10H), 2.98-2.57 (m, 0.8H), 2.42-1.79 (m, 4.8H), 1.77-1.50 (m, 1.2H), 1.45-1.31 (m, 0.6H), 1.14-0.98 (m, 0.6H).

With this procedure, reaction of sodium hyaluronate (MW 500 KDa) provided corresponding product (0.168 g, yield: 55.5%, DSR=25%). ¹H NMR (400 MHz, Deuterium Oxide) δ 7.75-7.61 (m, 0.25H), 6.62-6.52 (m, 0.25H), 6.42-6.32 (m, 0.25H), 5.56-5.40 (m, 0.25H), 5.25-5.07 (m, 0.5H), 4.84-4.46 (m, 2.5H), 4.26-3.32 (m, 10H), 2.98-2.58 (m, 1H), 2.42-1.78 (m, 5.25H), 1.77-1.51 (m, 1.5H), 1.45-1.30 (m, 0.75H), 1.14-0.95 (m, 0.75H).

With this procedure, reaction of sodium hyaluronate (MW 500 KDa) provided corresponding product (0.202 g, yield: 66.7%, DSR=10%). ¹H NMR (400 MHz, Deuterium Oxide) δ 7.74-7.63 (m, 0.1H), 6.63-6.52 (m, 0.1H), 6.42-6.34 (m, 0.1H), 5.53-5.10 (m, 0.3H), 4.82-4.47 (m, 2.2H), 4.27-3.27 (m, 10H), 2.94-2.57 (m, 0.4H), 2.42-1.78 (m, 3.9H), 1.75-1.58 (m, 0.6H), 1.43-1.34 (m, 0.3H), 1.11-0.98 (m, 0.3H).

### Example 9

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 8-aminooctanoate

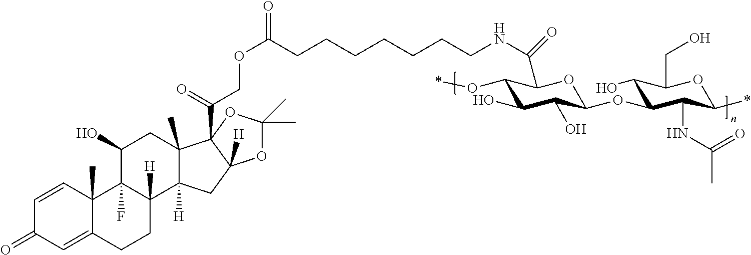

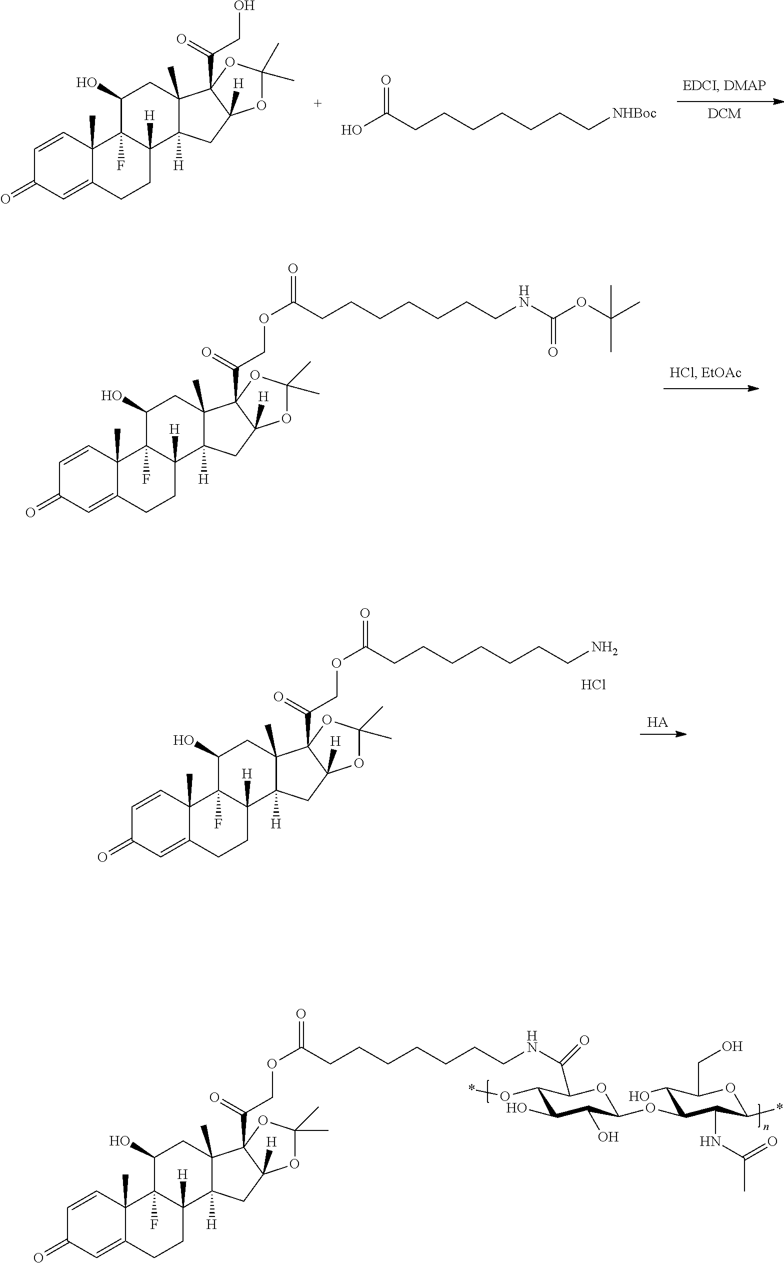

### Step 1: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 8-(((tert-butoxycarbonyl)amino)octanoate

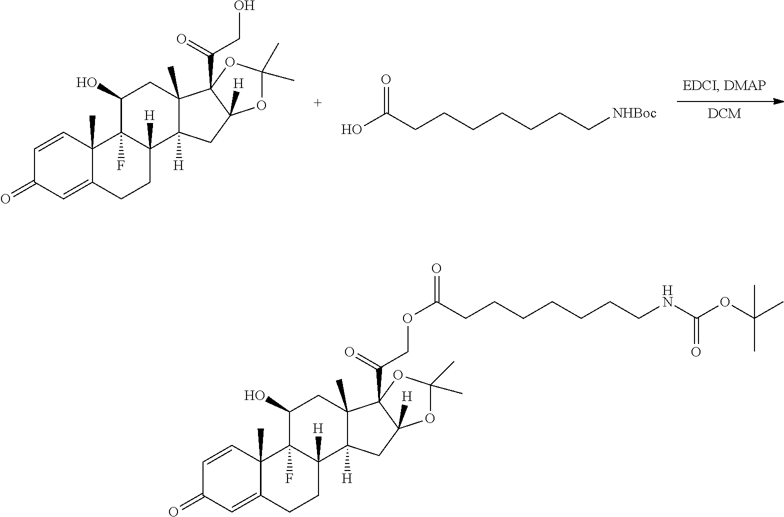

9α-Fluoro-11β,16α,17,21-tetrahydroxy-1,4-pregnadiene-3,20-dione (Triamcinolone acetonide, 652 mg, 1.5 mmol), 8-(((tert-butoxycarbonyl)amino)octanoic acid (506 mg, 1.95 mmol), 1-ethyl-3-(3-dimethylaminopropyl)carbodiimide (EDCI, 374 mg, 1.95 mmol), N,N-dimethylpyridin-4-amine (DMAP, 18 mg, 0.15 mmol) were dissolved in DCM (18 mL). The reaction mixture was stirred at room temperature for 24 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=2:1˜5:3) to afford the title compound (720 mg, Yield: 54.7%). MS (m/z): [M+H]+ calcd for C₃₇H₅₄FNO₉, 676.84; found, 620.2 (M+H−56). ¹H NMR (400 MHz, Chloroform-d) δ 7.21 (d, J=10.1 Hz, 1H), 6.34 (dd, J=10.1, 1.9 Hz, 1H), 6.13 (d, J=1.9 Hz, 1H), 5.03-4.95 (m, 1H), 4.95-4.78 (m, 2H), 4.55 (t, J=5.9 Hz, 1H), 4.42 (d, J=8.8 Hz, 1H), 3.09 (t, J=6.9 Hz, 2H), 2.70-2.57 (m, 1H), 2.55-2.32 (m, 5H), 2.16-2.05 (m, 1H), 1.92-1.82 (m, 1H), 1.78-1.60 (m, 6H), 1.55 (m, 3H), 1.44 (d, J=5.7 Hz, 14H), 1.39-1.29 (m, 7H), 1.22 (s, 3H), 0.95 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 8-aminooctanoate hydrochloride

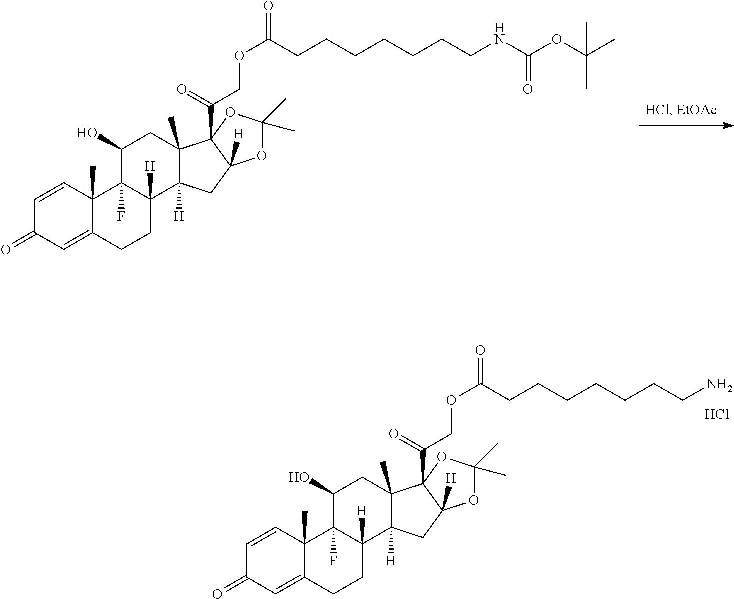

To a stirred solution of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 8-((tert-butoxycarbonyl)amino)octanoate (600 mg, 0.89 mmol) in EtOAc (24 mL) was slowly added 4M HCl in ethyl acetate (4.8 mL) at ice-bath. The reaction mixture was allowed to stir at room temperature for 18 hours. The solution was diluted with EtOAc and concentrated under reduced pressure to afford the title compound as a white solid (0.533 g, yield: 97.9%) that was used without further purification. MS (m/z): [M+H]+ calcd for C₃₂H₄₆FNO₇, 576.72; found, 576.1.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 8-aminooctanoate

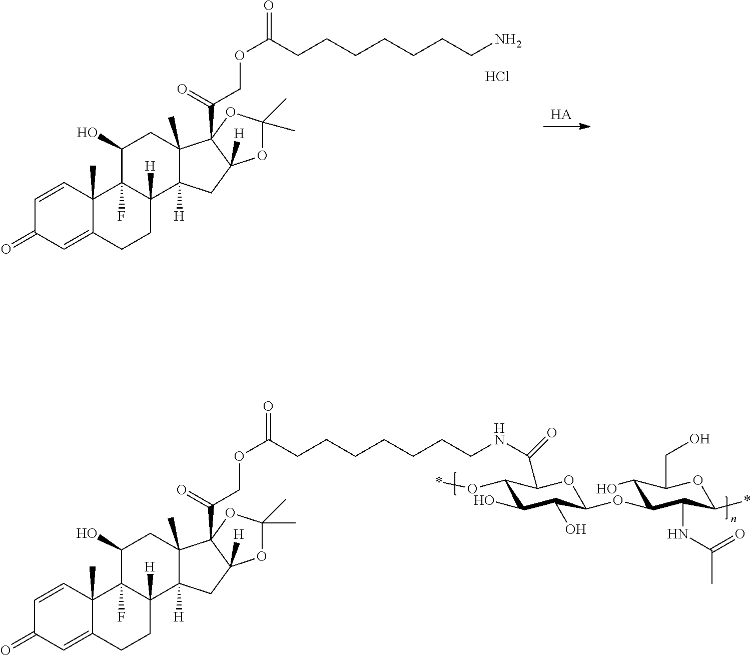

Hyaluronic acid (161 mg, 0.4 mmol carboxylic acid) was dissolved in 32 mL of deionized water in a 100 mL round-bottomed flask followed by the dropwise addition of 21 mL of acetonitrile while stirring. To the solution was added 4-methylmorpholine (NMM, 28 mg, 0.28 mmol) and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 8-aminooctanoate hydrochloride (172 mg, 0.28 mmol) causing the viscosity to increase temporarily. The solution was then cooled to 0° C., and 4-(4,6-Dimethoxy-1,3,5-triazin-2-yl)-4-methylmorpholinium chloride (DMTMM, 78 mg, 0.28 mmol) was added and stirred at room temperature for 72 hours at room temperature. NaCl (234 mg, 4 mmol) was then added to the reaction mixture, which was stirred for 1 hour and followed by the dropwise addition of anhydrous alcohol (200 mL) while stirring. The mixture was filtered. The filter cake was collected, washed with anhydrous alcohol and dried in vacuo to give the title compound as a white solid. (sodium hyaluronate MW 50 KDa 0.2 g, yield: 62.1%, DSR=8%); ¹H NMR (400 MHz, Deuterium Oxide) δ 7.65-7.56 (m, 0.08H), 6.57-6.46 (m, 0.08H), 6.34-6.26 (m, 0.08H), 5.35-5.25 (m, 0.08H), 5.17-5.01 (m, 0.16H), 4.83-4.43 (m, 2.08H), 4.28-3.07 (m, 10.16H), 2.85-2.52 (m, 0.08H), 2.35-1.69 (m, 4.04H), 1.67-1.50 (m, 0.72H), 1.48-1.36 (m, 0.72H), 1.05-0.92 (m, 0.24H).

With this procedure, reaction of sodium hyaluronate (MW 500 KDa) provided corresponding product (0.2 g, yield: 62.1%, DSR=12%). ¹H NMR (400 MHz, Deuterium Oxide) δ 7.70-7.58 (m, 0.12H), 6.60-6.50 (m, 0.12H), 6.41-6.30 (m, 0.12H), 5.41-5.28 (m, 0.12H), 5.20-5.02 (m, 0.24H), 4.79-4.42 (m, 2.12H), 4.31-3.08 (m, 10.24H), 2.90-2.48 (m, 0.12H), 2.38-1.74 (m, 4.56H), 1.70-1.54 (m, 1.08H), 1.50-1.39 (m, 1.08H), 1.08-0.94 (m, 0.36H).

With this procedure, reaction of sodium hyaluronate (MW 2000 KDa) provided corresponding product (0.121 g, yield: 37.5%, DSR=22%). ¹H NMR (400 MHz, Deuterium Oxide) δ 7.66-7.53 (m, 0.22H), 6.61-6.45 (m, 0.22H), 6.39-6.26 (m, 0.22H), 5.35-5.03 (m, 0.66H), 4.83-4.38 (m, 2.22H), 4.11-3.09 (m, 10.44H), 2.82-2.41 (m, 0.22H), 2.29-1.69 (m, 5.86H), 1.67-1.49 (m, 1.98H), 1.48-1.37 (m, 1.98H), 1.01-0.92 (m, 0.66H).

### Example 10

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl piperidine-4-carboxylate

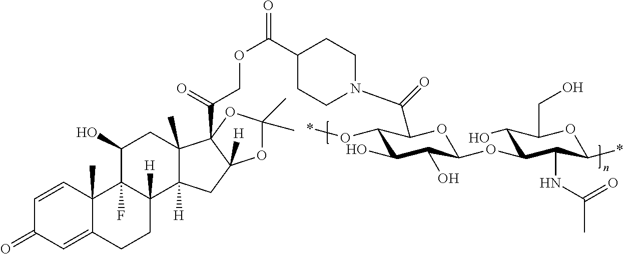

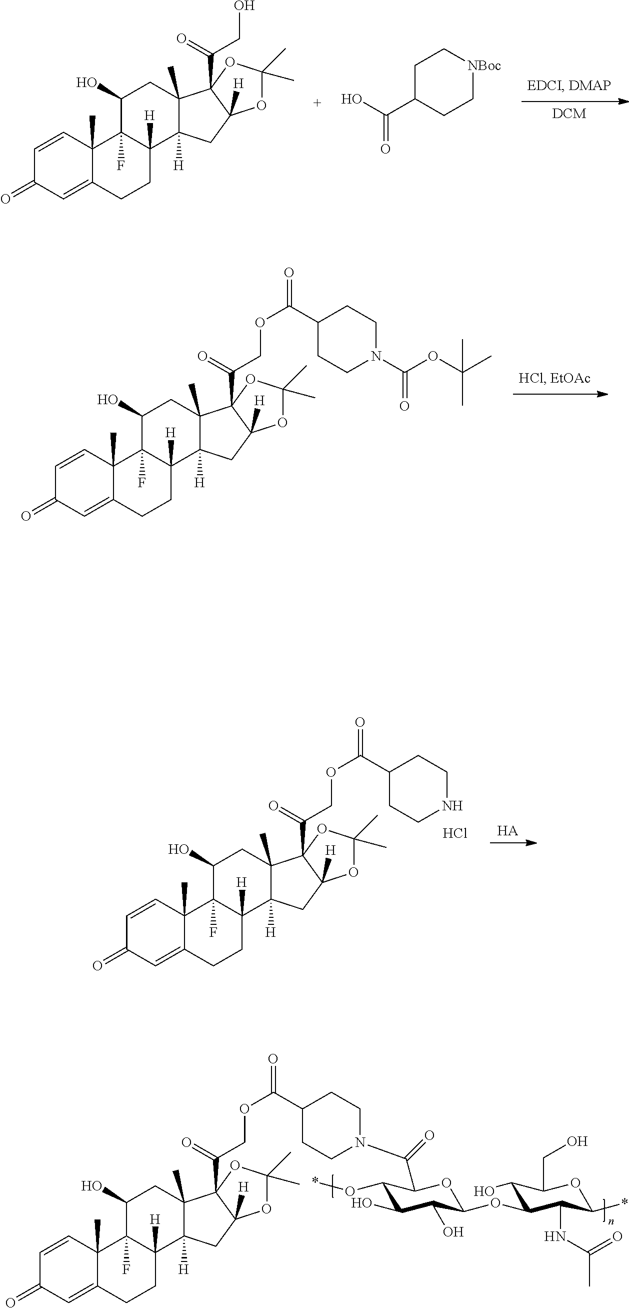

### Step 1: Preparation of 1-(tert-butyl) 4-(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) piperidine-1,4-dicarboxylate

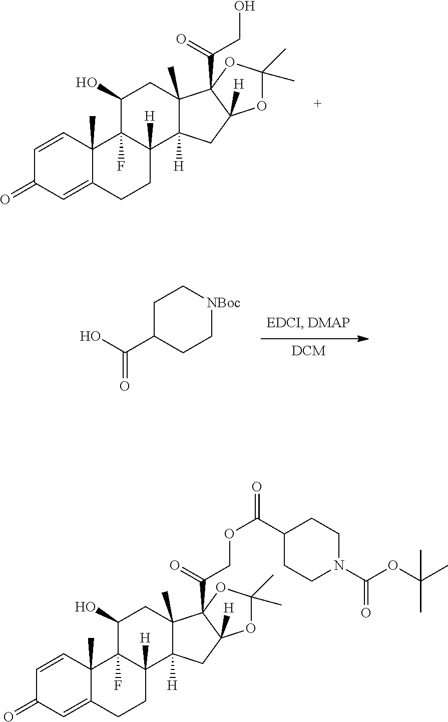

9α-Fluoro-11β,16α,17,21-tetrahydroxy-1,4-pregnadiene-3,20-dione (Triamcinolone acetonide, 652 mg, 1.5 mmol), 1-(tert-butoxycarbonyl)piperidine-4-carboxylic acid (447 mg, 1.95 mmol), 1-ethyl-3-(3-dimethylaminopropyl)carbodiimide (EDCI, 374 mg, 1.95 mmol), N,N-dimethylpyridin-4-amine (DMAP, 18 mg, 0.15 mmol) were dissolved in DCM (18 mL). The reaction mixture was stirred at room temperature for 24 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=5:1˜2:1) to afford the title compound (920 mg, Yield: 94.9%). MS (m/z): [M+H]⁺ calcd for C₃₅H₄₈FNO₉, 646.774; found, 590.3 (M+H−56). ¹H NMR (400 MHz, Chloroform-d) δ 7.22 (d, J=10.1 Hz, 1H), 6.35 (dd, J=10.1, 1.9 Hz, 1H), 6.13 (t, J=1.7 Hz, 1H), 5.07-4.92 (m, 2H), 4.86 (d, J=17.7 Hz, 1H), 4.42 (dq, J=8.9, 2.9 Hz, 1H), 4.01 (d, J=12.7 Hz, 2H), 2.91 (t, J=12.3 Hz, 2H), 2.62 (tt, J=10.1, 4.1 Hz, 2H), 2.55-2.27 (m, 4H), 2.10 (td, J=12.6, 5.9 Hz, 1H), 2.03-1.83 (m, 3H), 1.77-1.57 (m, 6H), 1.55 (s, 3H), 1.45 (d, J=10.9 Hz, 12H), 1.22 (s, 3H), 0.94 (s, 3H).

### Step 2: Preparation of 2-((6αS,6βR,7S,8αS,8βS,11αR,12αS,12βS)-6βb-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl piperidine-4-carboxylate hydrochloride

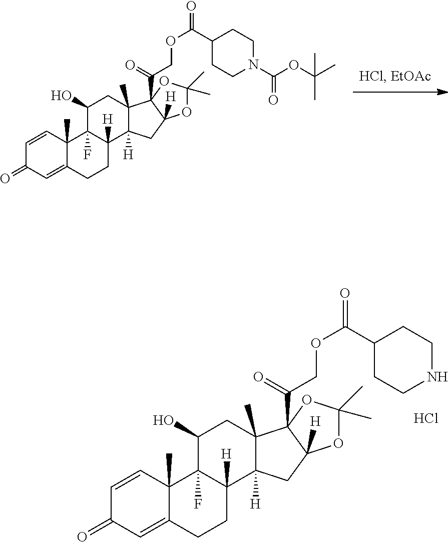

To a stirred solution of 1-(tert-butyl) 4-(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) piperidine-1,4-dicarboxylate (700 mg, 1.08 mmol) in EtOAc (28 mL) was slowly added 4M HCl in ethyl acetate (5.6 mL) at ice-bath. The reaction mixture was allowed to stir at room temperature for 40 hours. The solution was diluted with EtOAc and concentrated under reduced pressure to afford the title compound as a white solid (0.556 g, yield: 93.9%) that was used without further purification. MS (m/z): [M+H]+ calcd for C₃₀H₄₀FNO₇, 546.65; found, 546.4.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl piperidine-4-carboxylate

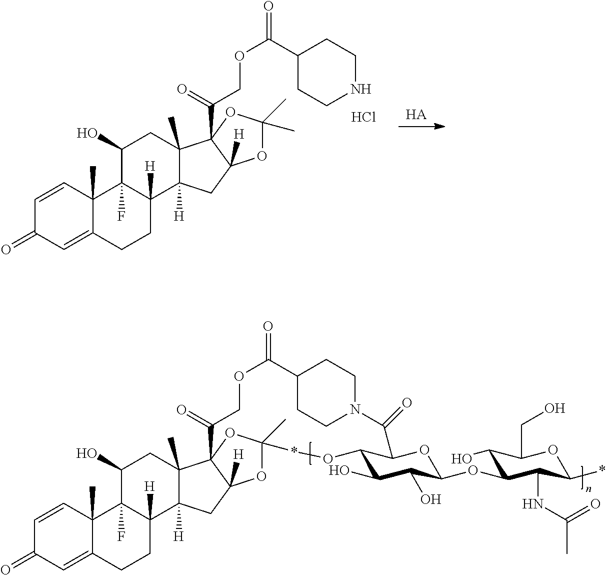

Hyaluronic acid (161 mg, 0.4 mmol carboxylic acid) was dissolved in 32 mL of deionized water in a 100 mL round-bottomed flask followed by the dropwise addition of 21 mL of acetonitrile while stirring. To the solution was added 4-methylmorpholine (NMM, 28 mg, 0.28 mmol) and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl piperidine-4-carboxylate hydrochloride (163 mg, 0.28 mmol) causing the viscosity to increase temporarily. The solution was then cooled to 0° C., and 4-(4,6-Dimethoxy-1,3,5-triazin-2-yl)-4-methylmorpholinium chloride (DMTMM, 78 mg, 0.28 mmol) was added and stirred at room temperature for 72 hours at room temperature. NaCl (234 mg, 4 mmol) was then added to the reaction mixture, which was stirred for 1 hour and followed by the dropwise addition of anhydrous alcohol (200 mL) while stirring. The mixture was filtered. The filter cake was collected, washed with anhydrous alcohol and dried in vacuo to give the title compound as a white solid. (sodium hyaluronate MW 50 KDa 0.2 g, yield: 63.7%, DSR=27%); ¹H NMR (400 MHz, Deuterium Oxide) δ 7.66-7.54 (m, 0.27H), 6.57-6.47 (m, 0.27H), 6.36-6.27 (m, 0.27H), 5.45-5.33 (m, 0.27H), 5.16-4.93 (m, 0.54H), 4.82-4.39 (m, 2.27H), 4.28-3.21 (m, 10.54H), 3.14-2.51 (m, 1.08H), 2.38-1.73 (m, 4.89H), 1.70-1.46 (m, 3.24H), 1.41-1.24 (m, 0.81H), 1.08-0.92 (m, 0.81H).

With this procedure, reaction of sodium hyaluronate (MW 500 KDa) provided corresponding product (0.18 g, yield: 57.3%, DSR=16%). ¹H NMR (400 MHz, Deuterium Oxide) δ 7.67-7.53 (m, 0.16H), 6.59-6.47 (m, 0.16H), 6.35-6.27 (m, 0.16H), 5.47-5.31 (m, 0.16H), 5.15-4.95 (m, 0.32H), 4.84-4.39 (m, 2.16H), 4.26-3.19 (m, 10.32H), 3.12-2.49 (m, 0.64H), 2.37-1.73 (m, 4.12H), 1.70-1.46 (m, 1.92H), 1.42-1.24 (m, 0.48H), 1.07-0.91 (m, 0.48H).

With this procedure, reaction of sodium hyaluronate (MW 2000 KDa) provided corresponding product (0.16 g, yield: 51%, DSR=5%). ¹H NMR (400 MHz, Deuterium Oxide) δ 7.67-7.56 (m, 0.05H), 6.57-6.49 (m, 0.05H), 6.35-6.29 (m, 0.05H), 5.45-5.33 (m, 0.05H), 5.18-4.98 (m, 0.1H), 4.83-4.39 (m, 2.05H), 4.22-3.17 (m, 10.1H), 2.95-2.39 (m, 0.2H), 2.32-1.71 (m, 3.35H), 1.68-1.50 (m, 0.6H), 1.41-1.27 (m, 0.15H), 1.05-0.90 (m, 0.15H).

### Example 11

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12βb-dodecahydro-8βH-naphtho[2′,1′:4,5]indeno[1,2-d][1,3]dioxol-8β-yl)-2-oxoethyl 3-aminocyclobutane-1-carboxylate

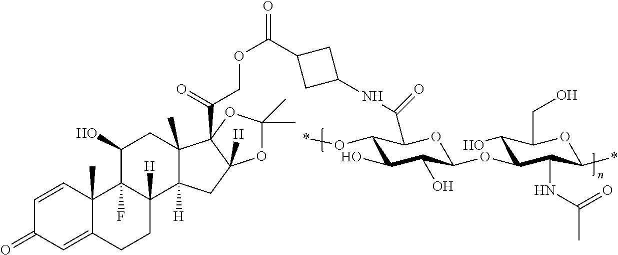

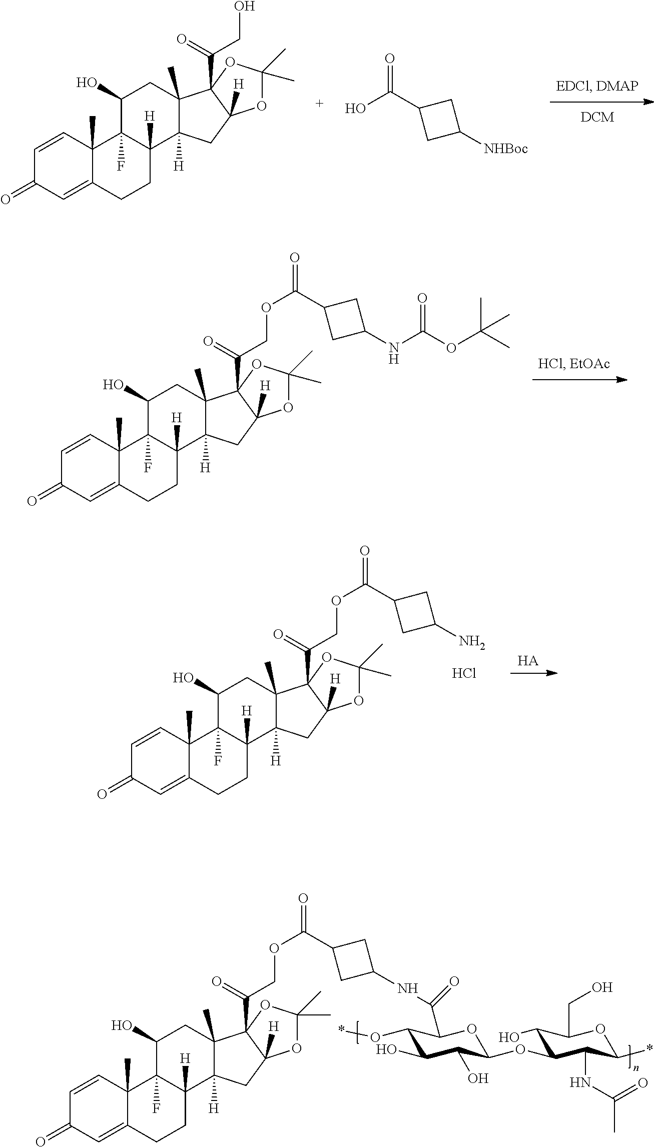

### Step 1: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-((tert-butoxycarbonyl)amino)cyclobutane-1-carboxylate

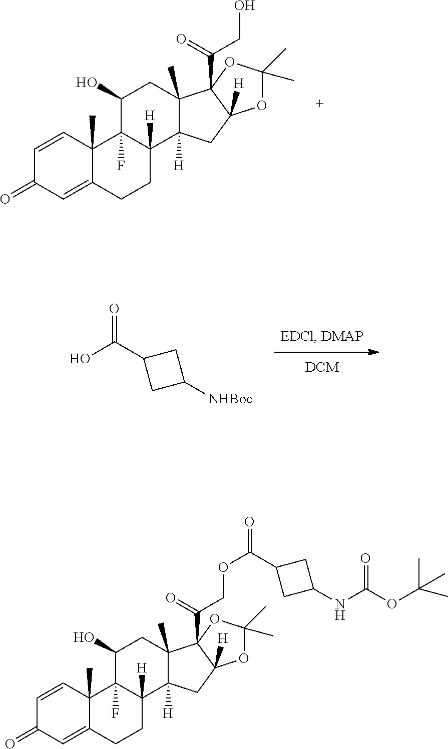

9α-Fluoro-11β,16α,17,21-tetrahydroxy-1,4-pregnadiene-3,20-dione (Triamcinolone acetonide, 1004 mg, 2.31 mmol), 3-((tert-butoxycarbonyl)amino)cyclobutane-1-carboxylic acid (3649 mg, 3 mmol), 1-ethyl-3-(3-dimethylaminopropyl)carbodiimide (EDCI, 663 mg, 3.46 mmol), N,N-dimethylpyridin-4-amine (DMAP, 28.3 mg, 0.23 mmol) were dissolved in DCM (30 mL). The reaction mixture was stirred at room temperature for 24 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=5:1˜2:1) to afford the title compound (1420 mg, Yield: 97.5%). MS (m/z): [M+H]⁺ calcd for C₃₄H₄₆FNO₉, 632.74; found, 576.3 (M+H−56). ¹H NMR (400 MHz, DMSO-d₆) δ 7.30 (d, J=10.2 Hz, 1H), 6.24 (dd, J=10.1, 1.9 Hz, 1H), 6.02 (t, J=1.7 Hz, 1H), 5.48 (dt, J=4.8, 2.1 Hz, 1H), 5.15 (dd, J=17.9, 11.2 Hz, 1H), 4.87 (t, J=5.4 Hz, 1H), 4.76 (t, J=18.2 Hz, 1H), 4.27-4.16 (m, 1H), 4.00-3.83 (m, 1H), 3.10-2.82 (m, 1H), 2.69-2.57 (m, 1H), 2.47-2.19 (m, 4H), 2.16-2.01 (m, 3H), 1.97-1.68 (m, 3H), 1.64-1.45 (m, 5H), 1.43-1.32 (m, 13H), 1.24 (s, 1H), 1.16 (d, J=4.8 Hz, 3H), 0.84 (d, J=6.9 Hz, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-aminocyclobutane-1-carboxylate hydrochloride

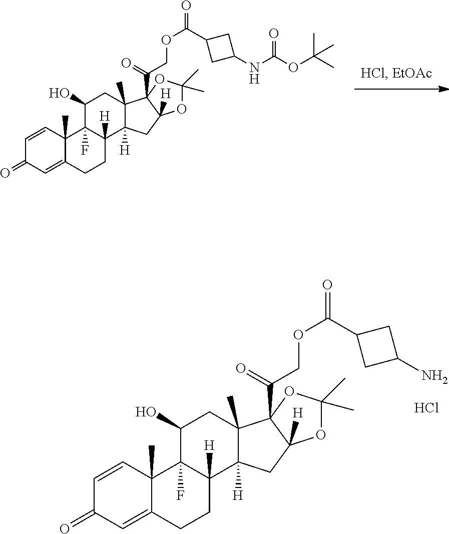

To a stirred solution of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-((tert-butoxycarbonyl)amino)cyclobutane-1-carboxylate (900 mg, 1.42 mmol) in EtOAc (36 mL) was slowly added 4M HCl in ethyl acetate (commercially available) (7.2 mL) at ice-bath. The reaction mixture was allowed to stir at room temperature for 40 hours. The solution was diluted with EtOAc and concentrated under reduced pressure to afford the title compound as a white solid (0.7 g, yield: 92.8%) that was used without further purification. MS (m/z): [M+H]+ calcd for C₂₉H₃₈FNO₇, 532.62; found, 532.2.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12βb-dodecahydro-8βH-naphtho[2′,1′:4,5]indeno[1,2-d][1,3]dioxol-8β-yl)-2-oxoethyl 3-aminocyclobutane-1-carboxylate

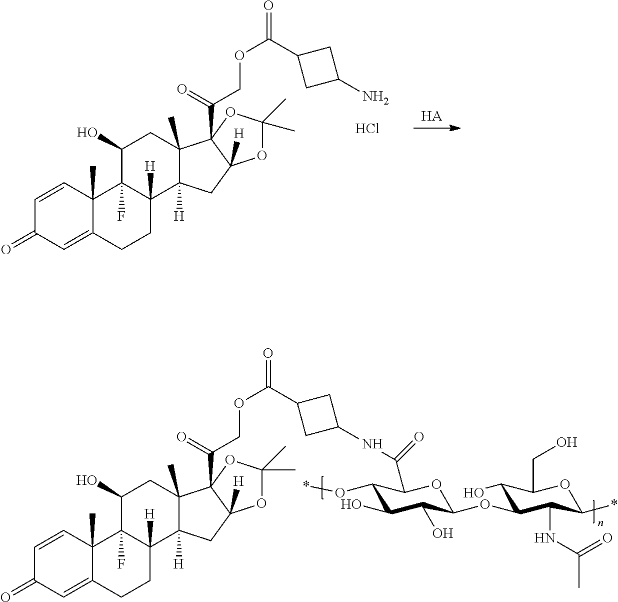

Hyaluronic acid (201 mg, 0.5 mmol carboxylic acid) was dissolved in 40 mL of deionized water in a 100 mL round-bottomed flask followed by the dropwise addition of 20 mL of acetonitrile while stirring. To the solution was added 4-methylmorpholine (NMM, 35 mg, 0.35 mmol) and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12βb-dodecahydro-8βH-naphtho[2′,1′:4,5]indeno[1,2-d][1,3]dioxol-8β-yl)-2-oxoethyl 3-aminocyclobutane-1-carboxylate hydrochloride (199 mg, 0.35 mmol) causing the viscosity to increase temporarily. The solution was then cooled to 0° C., and 4-(4,6-Dimethoxy-1,3,5-triazin-2-yl)-4-methylmorpholinium chloride (DMTMM, 97 mg, 0.35 mmol) was added and stirred at room temperature for 72 hours at room temperature. NaCl (439 mg, 7.5 mmol) was then added to the reaction mixture, which was stirred for 1 hour and followed by the dropwise addition of anhydrous alcohol (200 mL) while stirring. The mixture was filtered. The filter cake was collected, washed with anhydrous alcohol and dried in vacuo to give the title compound as a white solid. (sodium hyaluronate MW 50 KDa 0.18 g, yield: 46.5%, DSR=35%); ¹H NMR (400 MHz, Deuterium Oxide) δ 7.68-7.57 (m, 0.35H), 6.59-6.47 (m, 0.35H), 6.36-6.29 (m, 0.35H), 5.45-5.30 (m, 0.35H), 5.18-4.96 (m, 0.7H), 4.81-4.33 (m, 2.35H), 4.21-3.11 (m, 10.35H), 2.95-2.46 (m, 0.7H), 2.33-1.72 (m, 6.85H), 1.69-1.46 (m, 2.8H), 1.39-1.20 (m, 1.05H), 1.04-0.89 (m, 1.05H).

With this procedure, reaction of sodium hyaluronate (MW 500 KDa) provided corresponding product (0.19 g, yield: 49%, DSR=34%). ¹H NMR (400 MHz, Deuterium Oxide) δ 7.68-7.58 (m, 0.34H), 6.57-6.46 (m, 0.34H), 6.38-6.26 (m, 0.34H), 5.41-5.30 (m, 0.34H), 5.18-4.97 (m, 0.68H), 4.80-4.35 (m, 2.34H), 4.19-3.15 (m, 10.34H), 2.95-2.52 (m, 0.68H), 2.33-1.72 (m, 6.74H), 1.69-1.45 (m, 2.72H), 1.39-1.18 (m, 1.02H), 1.07-0.90 (m, 1.02H).

With this procedure, reaction of sodium hyaluronate (MW 2000 KDa) provided corresponding product (0.13 g, yield: 33.5%, DSR=25%). ¹H NMR (400 MHz, Deuterium Oxide) δ 7.70-7.56 (m, 0.25H), 6.61-6.48 (m, 0.25H), 6.37-6.23 (m, 0.25H), 5.42-5.30 (m, 0.25H), 5.18-4.97 (m, 0.5H), 4.80-4.43 (m, 2.25H), 4.22-3.07 (m, 10.25H), 2.93-2.60 (m, 0.5H), 2.38-1.92 (m, 5.75H), 1.72-1.43 (m, 2H), 1.40-1.19 (m, 0.75H), 1.06-0.87 (m, 0.75H).

### Example 12

### Preparation of Conjugate of HA and 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-aminopropanoate

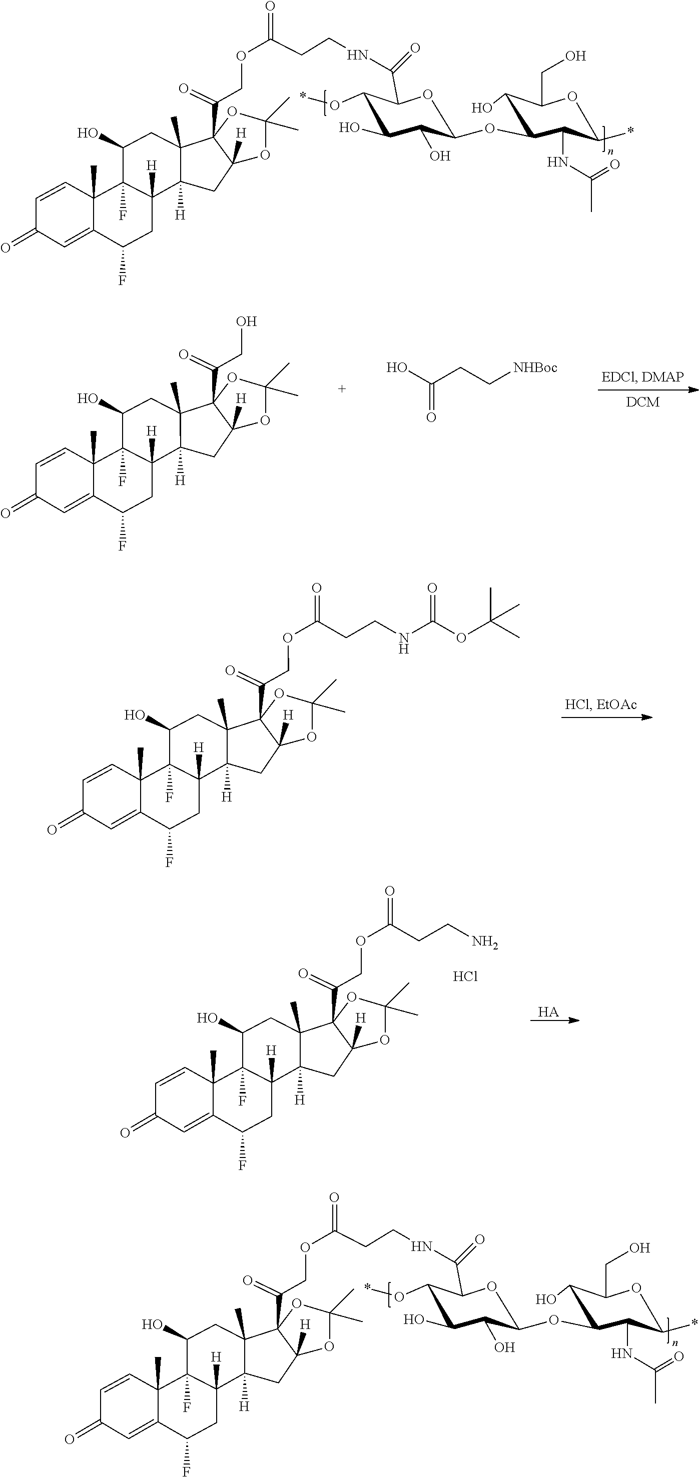

### Step 1: Preparation of 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-((tert-butoxycarbonyl)amino)propanoate

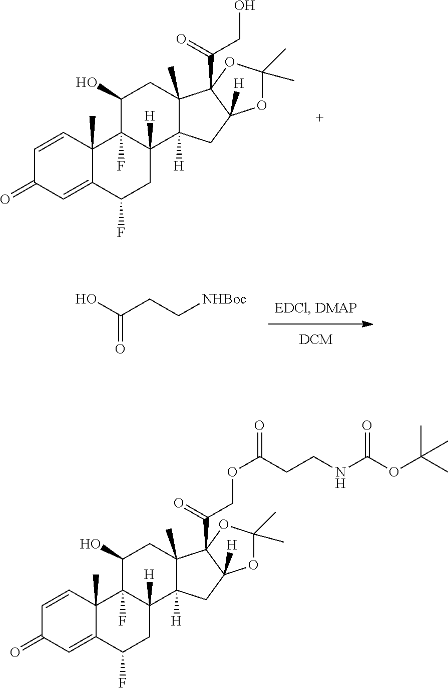

(6α,9α,11β,16β)-6,9-difluoro-11,21-dihydroxy-16,17-[(1-methylethylidene)bis(oxy)]pregna-1,4-diene-3,20-dione (Fluocinolone acetonide, 453 mg, 1 mmol), 4-((tert-butoxycarbonyl)amino)butanoic acid (246 mg, 1.3 mmol), 1-ethyl-3-(3-dimethylaminopropyl)carbodiimide (EDCI, 249 mg, 1.3 mmol), N,N-dimethylpyridin-4-amine (DMAP, 12 mg, 0.1 mmol) were dissolved in DCM (20 mL). The reaction mixture was stirred at room temperature for 24 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=5:1˜2:1) to afford the title compound (620 mg, Yield: 99.5%). MS (m/z): [M+H]⁺ calcd for C₃₂H₄₃F₂NO₉, 624.69; found, 568.2 (M+H−56). ¹H NMR (400 MHz, Chloroform-d) δ 7.15 (dd, J=10.2, 1.5 Hz, 1H), 6.44 (d, J=2.0 Hz, 1H), 6.38 (dd, J=10.1, 1.9 Hz, 1H), 5.53-5.29 (m, 1H), 5.17 (s, 1H), 5.07-4.85 (m, 3H), 4.43 (dq, J=8.8, 2.8 Hz, 1H), 3.46 (q, J=6.2 Hz, 2H), 2.66 (t, J=6.1 Hz, 2H), 2.58-2.34 (m, 3H), 2.34-2.24 (m, 1H), 2.19 (td, J=12.4, 5.8 Hz, 1H), 1.88-1.59 (m, 4H), 1.54 (s, 3H), 1.51-1.39 (m, 12H), 1.22 (s, 3H), 0.93 (s, 3H).

### Step 2: Preparation of 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-aminopropanoate hydrochloride

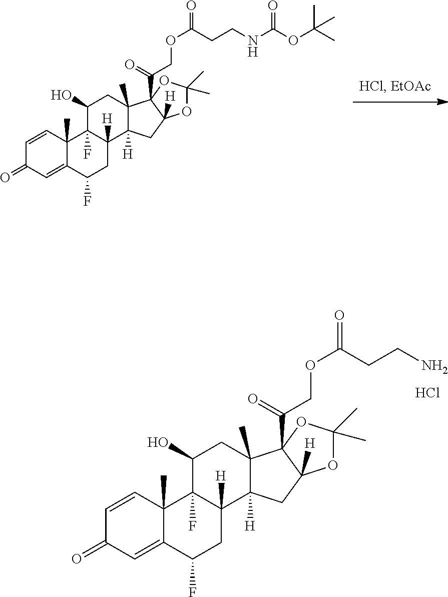

To a stirred solution of 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-((tert-butoxycarbonyl)amino)propanoate (400 mg, 0.64 mmol) in EtOAc (16 mL) was slowly added 4M HCl in ethyl acetate (3.2 mL) at ice-bath. The reaction mixture was allowed to stir at room temperature for 40 hours. The solution was diluted with EtOAc and concentrated under reduced pressure to afford the title compound as a white solid (0.375 g, yield: 97.9%) that was used without further purification. MS (m/z): [M+H]+ calcd for C₂₇H₃₅F₂NO₇, 524.57; found, 524.3

### Step 3: Preparation of Conjugate of HA and 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-aminopropanoate

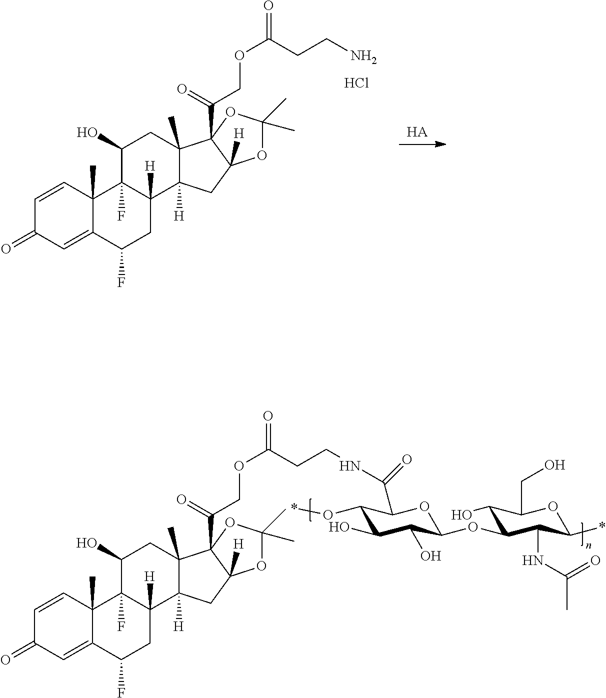

Hyaluronic acid (161 mg, 0.4 mmol carboxylic acid) was dissolved in 32 mL of deionized water in a 100 mL round-bottomed flask followed by the dropwise addition of 21 mL of acetonitrile while stirring. To the solution was added 4-methylmorpholine (NMM, 28 mg, 0.28 mmol) and 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 3-aminopropanoate hydrochloride (157 mg, 0.28 mmol) causing the viscosity to increase temporarily. The solution was then cooled to 0° C., and 4-(4,6-Dimethoxy-1,3,5-triazin-2-yl)-4-methylmorpholinium chloride (DMTMM, 78 mg, 0.28 mmol) was added and stirred at room temperature for 72 hours at room temperature. NaCl (234 mg, 4 mmol) was then added to the reaction mixture, which was stirred for 1 hour and followed by the dropwise addition of anhydrous alcohol (200 mL) while stirring. The mixture was filtered. The filter cake was collected, washed with anhydrous alcohol and dried in vacuo to give the title compound as a white solid. (sodium hyaluronate MW 50 KDa 0.198 g, yield: 64.4%, DSR=30%); ¹H NMR (400 MHz, Deuterium Oxide) δ 7.67-7.54 (m, 0.3H), 6.67-6.49 (m, 0.6H), 5.91-5.63 (m, 0.3H), 5.44-5.32 (m, 0.3H), 5.23-5.01 (m, 0.6H), 4.81-4.45 (m, 2.3H), 4.21-3.31 (m, 10.6H), 3.02-2.47 (m, 0.9H), 2.38-1.74 (m, 4.5H), 1.72-1.49 (m, 2.4H), 1.42-1.27 (m, 1.5H), 1.09-0.90 (m, 1.5H).

### Example 13

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-(aminomethyl)benzoate

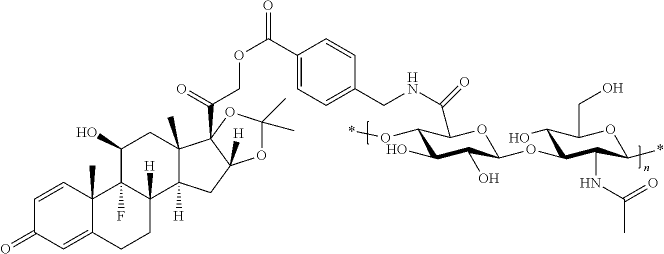

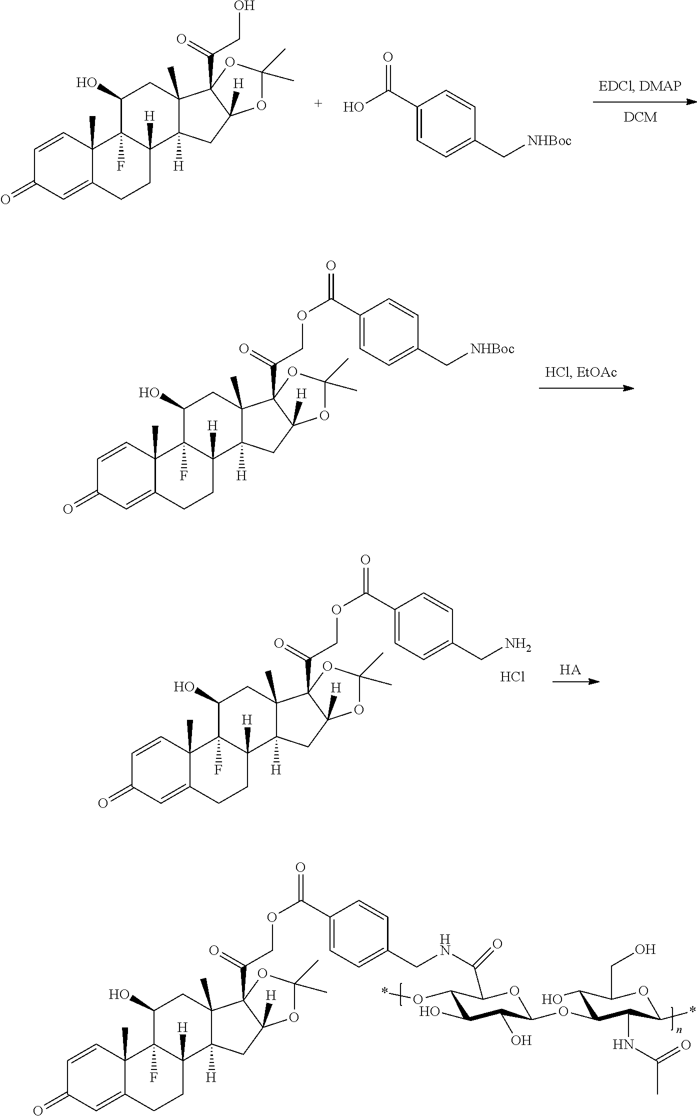

### Step 1: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-(((tert-butoxycarbonyl)amino)methyl)benzoate

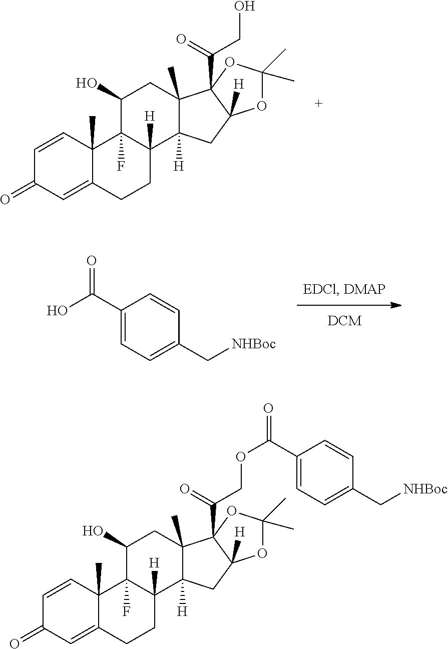

9α-Fluoro-11β,16α,17,21-tetrahydroxy-1,4-pregnadiene-3,20-dione (Triamcinolone acetonide, 679 mg, 1.56 mmol), 4-(((tert-butoxycarbonyl)amino)methyl)benzoic acid (387.5 mg, 1.8 mmol), 1-ethyl-3-(3-dimethylaminopropyl)carbodiimide (EDCI, 432 mg, 2.25 mmol), N,N-dimethylpyridin-4-amine (DMAP, 18 mg, 0.15 mmol) were dissolved in DCM (20 mL). The reaction mixture was stirred at room temperature for 24 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=5:1˜2:1) to afford the title compound (650 mg, Yield: 62.4%). MS (m/z): [M+H]⁺ calcd for C₃₇H₄₆FNO₉, 668.77; found, 668.2. ¹H NMR (400 MHz, DMSO-d₆) δ 7.97 (d, J=8.0 Hz, 2H), 7.42 (d, J=8.0 Hz, 2H), 7.31 (d, J=10.2 Hz, 1H), 6.25 (dd, J=10.1, 1.9 Hz, 1H), 6.03 (t, J=1.7 Hz, 1H), 5.51 (d, J=4.1 Hz, 1H), 5.41 (d, J=18.0 Hz, 1H), 5.01 (d, J=17.9 Hz, 1H), 4.89 (d, J=4.6 Hz, 1H), 4.22 (d, J=6.1 Hz, 3H), 2.73-2.60 (m, 1H), 2.58-2.42 (m, 2H), 2.39-2.30 (m, 1H), 2.15-2.06 (m, 1H), 2.03-1.92 (m, 2H), 1.82 (t, J=8.9 Hz, 2H), 1.54 (d, J=21.4 Hz, 5H), 1.40 (d, J=8.5 Hz, 12H), 1.23 (s, 3H), 0.90 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-(aminomethyl)benzoate hydrochloride

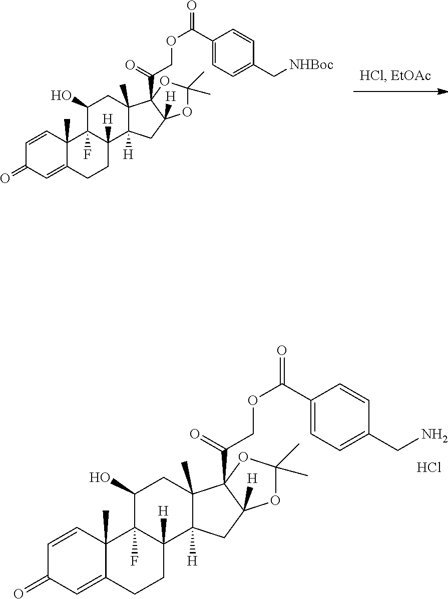

To a stirred solution of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-(((tert-butoxycarbonyl)amino)methyl)benzoate (200 mg, 0.3 mmol) in EtOAc (20 mL) was slowly added 4M HCl in ethyl acetate (4 mL) at ice-bath. The reaction mixture was allowed to stir at room temperature for 40 hours. The solution was diluted with EtOAc and concentrated under reduced pressure to afford the title compound as a white solid (0.18 g, yield: 99%) that was used without further purification. MS (m/z): [M+H]+ calcd for C₃₂H₃₈FNO₇, 568.65; found, 568.3.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-(aminomethyl)benzoate

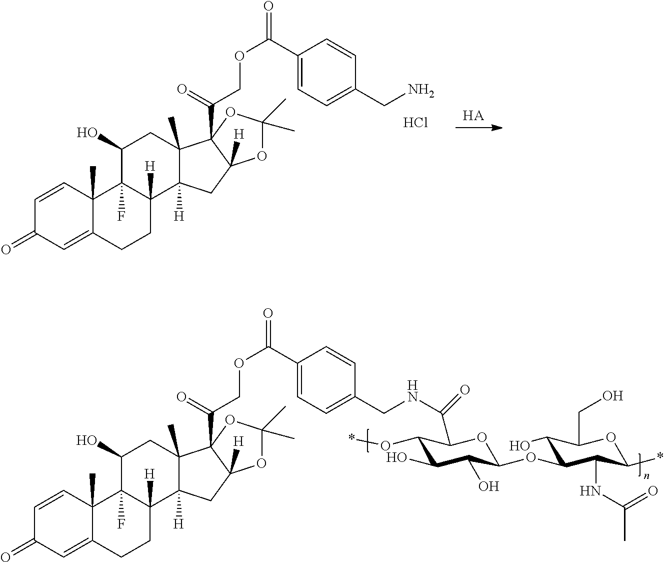

Hyaluronic acid (201 mg, 0.5 mmol carboxylic acid) was dissolved in 22 mL of deionized water in a 100 mL round-bottomed flask followed by the dropwise addition of 22 mL of 1,4-Dioxane while stirring. To the solution was added 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-(aminomethyl)benzoate hydrochloride (120 mg, 0.2 mmol), N-Hydroxysuccinimide (NHS, 46 mg, 0.4 mmol) and 1-ethyl-3-(3-dimethylaminopropyl)carbodiimide (EDCI, 38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (5 mL) was added to the reaction mixture, after stirring for 1 hour, 1M HCl solution (4.3 mL) was added. NaCl (293 mg, 5 mmol) was then added to the reaction mixture, which was stirred for 1 hour and followed by the dropwise addition of anhydrous alcohol (200 mL) while stirring at −10° C. The mixture was filtered. The filter cake was collected, washed with anhydrous alcohol and dried in vacuo to give the title compound as a white solid. (sodium hyaluronate MW 50 KDa 0.2 g, yield: 63.4%, DSR=30%); ¹H NMR (400 MHz, Deuterium Oxide) δ 8.24-8.12 (m, 0.6H), 7.72-7.57 (m, 0.9H), 6.62-6.50 (m, 0.3H), 6.40-6.30 (m, 0.3H), 5.65-5.56 (m, 0.3H), 5.38-5.16 (m, 0.9H), 4.78-4.43 (m, 2.6H), 4.23-3.24 (m, 10.9H), 2.93-2.49 (m, 1.2H), 2.45-1.77 (m, 4.2H), 1.72-1.54 (m, 2.4H), 1.49-1.38 (m, 0.9H), 1.14-0.97 (m, 0.9H).

With this procedure, reaction of sodium hyaluronate (MW 500 KDa) provided corresponding product (0.13 g, yield: 41.2%, DSR=25%). ¹H NMR (400 MHz, Deuterium Oxide) δ 8.27-8.13 (m, 0.5H), 7.76-7.57 (m, 0.75H), 6.64-6.50 (m, 0.25H), 6.41-6.26 (m, 0.25H), 5.71-5.56 (m, 0.25H), 5.39-5.13 (m, 0.75H), 4.80-4.43 (m, 2.5H), 4.21-3.18 (m, 10.75H), 2.99-2.54 (m, 1H), 2.44-1.77 (m, 4H), 1.75-1.55 (m, 2H), 1.50-1.38 (m, 0.75H), 1.18-0.99 (m, 0.75H).

With this procedure, reaction of sodium hyaluronate (MW 2000 KDa) provided corresponding product (0.13 g, yield: 41.2%, DSR=25%). ¹H NMR (400 MHz, Deuterium Oxide) δ 8.26-8.12 (m, 0.5H), 7.74-7.55 (m, 0.75H), 6.65-6.50 (m, 0.25H), 6.40-6.27 (m, 0.25H), 5.70-5.51 (m, 0.25H), 5.36-5.09 (m, 0.75H), 4.84-4.41 (m, 2.5H), 4.27-3.08 (m, 10.75H), 2.97-2.54 (m, 1H), 2.43-1.78 (m, 4H), 1.75-1.56 (m, 2H), 1.50-1.37 (m, 0.75H), 1.17-0.99 (m, 0.75H).

### Example 14

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (2-aminoethyl)carbamate

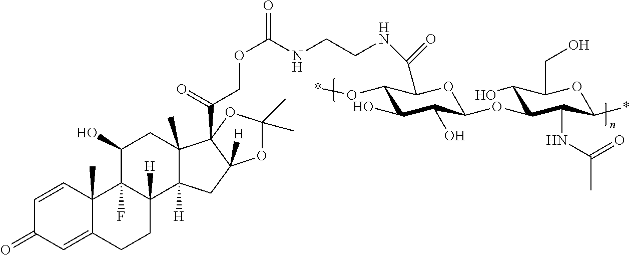

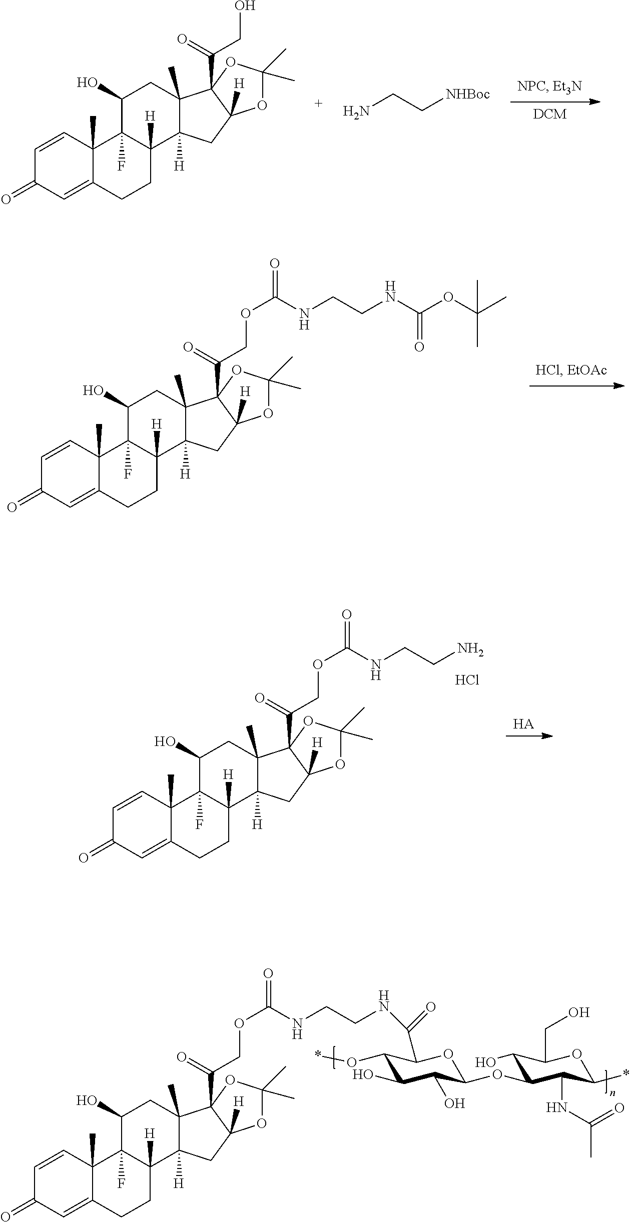

### Step 1: Preparation of tert-butyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) ethane-1,2-diyldicarbamate

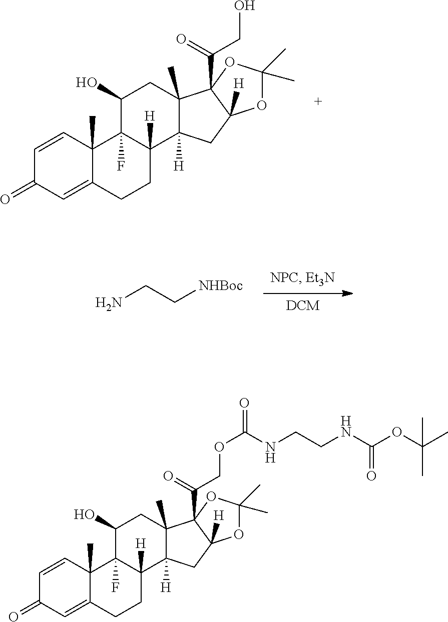

To a mixture of 9α-Fluoro-11β,16α,17,21-tetrahydroxy-1,4-pregnadiene-3,20-dione (Triamcinolone acetonide, 652 mg, 1.5 mmol), and Bis (4-nitrophenyl) carbonate (547 mg, 1.8 mmol) in dichloromethane (80 mL) was added triethylamine (0.42 mL, 3 mmol) under N₂, the reaction mixture was heated to reflux for 2 hours. Then tert-butyl (2-aminoethyl)carbamate (312 mg, 1.95 mmol) was added and the resulting mixture was stirred at room temperature for 2 hours. The solution was diluted with DCM and washed with saturated NaHCO₃ solution, water and saturated brine solution. The organic layer was dried over sodium sulfate, filtered and then concentrated under reduced pressure. The residue was purified by silical gel chromatography to give the title product (0.65 g, yield: 69.8%); MS (m/z): [M+H]⁺ calcd for C₃₂H₄₅FN₂O₉, 621.72; found, 565.3 (M+H−56). ¹H NMR (400 MHz, Chloroform-d) δ 7.22 (d, J=10.2 Hz, 1H), 6.34 (dd, J=10.1, 1.9 Hz, 1H), 6.13 (t, J=1.8 Hz, 1H), 5.56-5.32 (m, 1H), 4.99 (d, J=4.9 Hz, 1H), 4.94-4.80 (d, J=16.9 Hz, 3H), 4.45-4.37 (m, 1H), 3.40-3.20 (m, 4H), 2.72-2.56 (m, 1H), 2.56-2.32 (m, 4H), 2.17-2.03 (m, 2H), 1.95-1.82 (m, 3H), 1.75 (d, J=14.0 Hz, 1H), 1.70-1.59 (m, 3H), 1.45 (s, 9H), 1.43 (s, 3H), 1.21 (s, 3H), 0.96 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (2-aminoethyl)carbamate hydrochloride

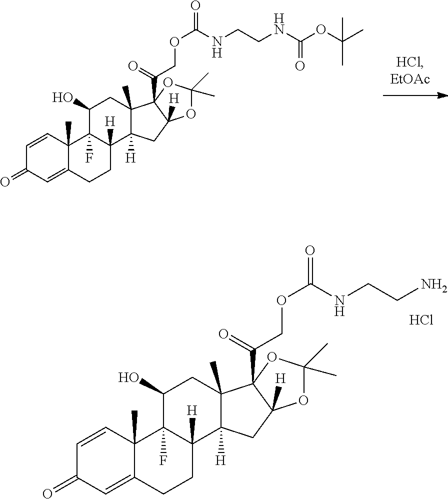

To a stirred solution of tert-butyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) ethane-1,2-diyldicarbamate (500 mg, 0.8 mmol) in EtOAc (20 mL) was slowly added 4M HCl in ethyl acetate (4 mL) at ice-bath. The reaction mixture was allowed to stir at room temperature for 40 hours. The solution was diluted with EtOAc and concentrated under reduced pressure to afford the title compound as a white solid (0.386 g, yield: 97.9%) that was used without further purification. MS (m/z): [M+H]⁺ calcd for C₂₇H₃₇FN₂O₇, 521.60; found, 521.3

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (2-aminoethyl)carbamate

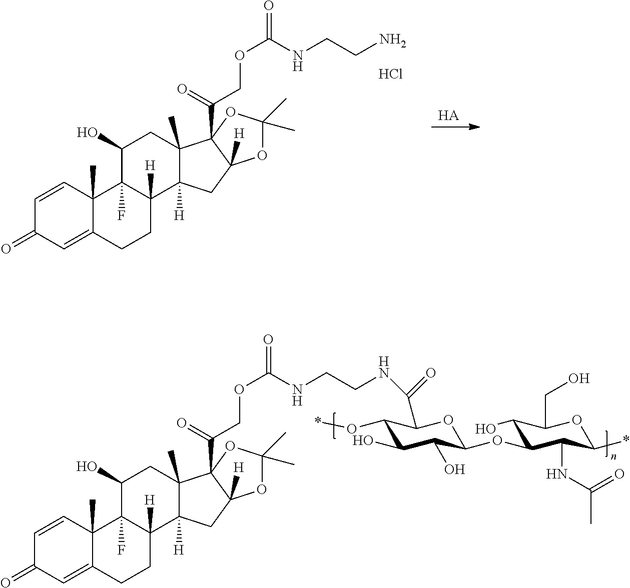

Hyaluronic acid (201 mg, 0.5 mmol carboxylic acid) was dissolved in 22 mL of deionized water in a 100 mL round-bottomed flask followed by the dropwise addition of 22 mL of 1,4-Dioxane while stirring. To the solution was added 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (2-aminoethyl)carbamate hydrochloride (111 mg, 0.2 mmol), N-Hydroxysuccinimide (NHS, 46 mg, 0.4 mmol) and 1-ethyl-3-(3-dimethylaminopropyl)carbodiimide (EDCI, 38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (5 mL) was added to the reaction mixture, after stirring for 1 hour, 1M HCl solution (4.3 mL) was added. NaCl (1000 mg, 17 mmol) was then added to the reaction mixture, which was stirred for 1 hour and followed by the dropwise addition of anhydrous alcohol (200 mL) while stirring at −10° C. The mixture was filtered. The filter cake was collected, washed with anhydrous alcohol and dried in vacuo to give the title compound as a white solid. (sodium hyaluronate MW 50 KDa 0.2 g, yield: 65.5%, DSR=18%); ¹H NMR (400 MHz, Deuterium Oxide) δ 7.68-7.58 (m, 0.18H), 6.59-6.48 (m, 0.18H), 6.37-6.29 (m, 0.18H), 5.40-5.24 (m, 0.18H), 5.21-5.09 (m, 0.18H), 4.82-4.40 (m, 2.54H), 4.26-3.13 (m, 10.72H), 2.95-2.51 (m, 0.72H), 2.37-1.72 (m, 4.08H), 1.71-1.47 (m, 1.08H), 1.40-1.30 (m, 0.54H), 1.09-0.92 (m, 0.54H).

With this procedure, reaction of sodium hyaluronate (MW 500 KDa) provided corresponding product (0.2 g, yield: 65.5%, DSR=19%). ¹H NMR (400 MHz, Deuterium Oxide) δ 7.70-7.58 (m, 0.19H), 6.61-6.52 (m, 0.19H), 6.41-6.30 (m, 0.19H), 5.44-5.24 (m, 0.19H), 5.22-5.09 (m, 0.19H), 4.81-4.40 (m, 2.38H), 4.27-3.11 (m, 10.76H), 2.96-2.53 (m, 0.76H), 2.41-1.73 (m, 4.14H), 1.71-1.48 (m, 1.14H), 1.44-1.31 (m, 0.57H), 1.10-0.95 (m, 0.57H).

With this procedure, reaction of sodium hyaluronate (MW 2000 KDa) provided corresponding product (0.19 g, yield: 62.2%, DSR=14%). ¹H NMR (400 MHz, Deuterium Oxide) δ 7.69-7.56 (m, 0.14H), 6.61-6.49 (m, 0.14H), 6.40-6.28 (m, 0.14H), 5.44-5.24 (m, 0.14H), 5.22-5.07 (m, 0.14H), 4.81-4.39 (m, 2.28H), 4.26-3.11 (m, 10.56H), 2.92-2.49 (m, 0.56H), 2.39-1.75 (m, 3.84H), 1.72-1.48 (m, 0.84H), 1.43-1.30 (m, 0.42H), 1.09-0.93 (m, 0.42H).

### Example 15

### Preparation of Conjugate of HA and 4-(aminomethyl)benzyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

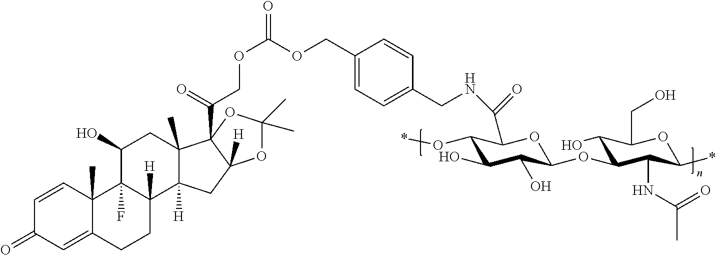

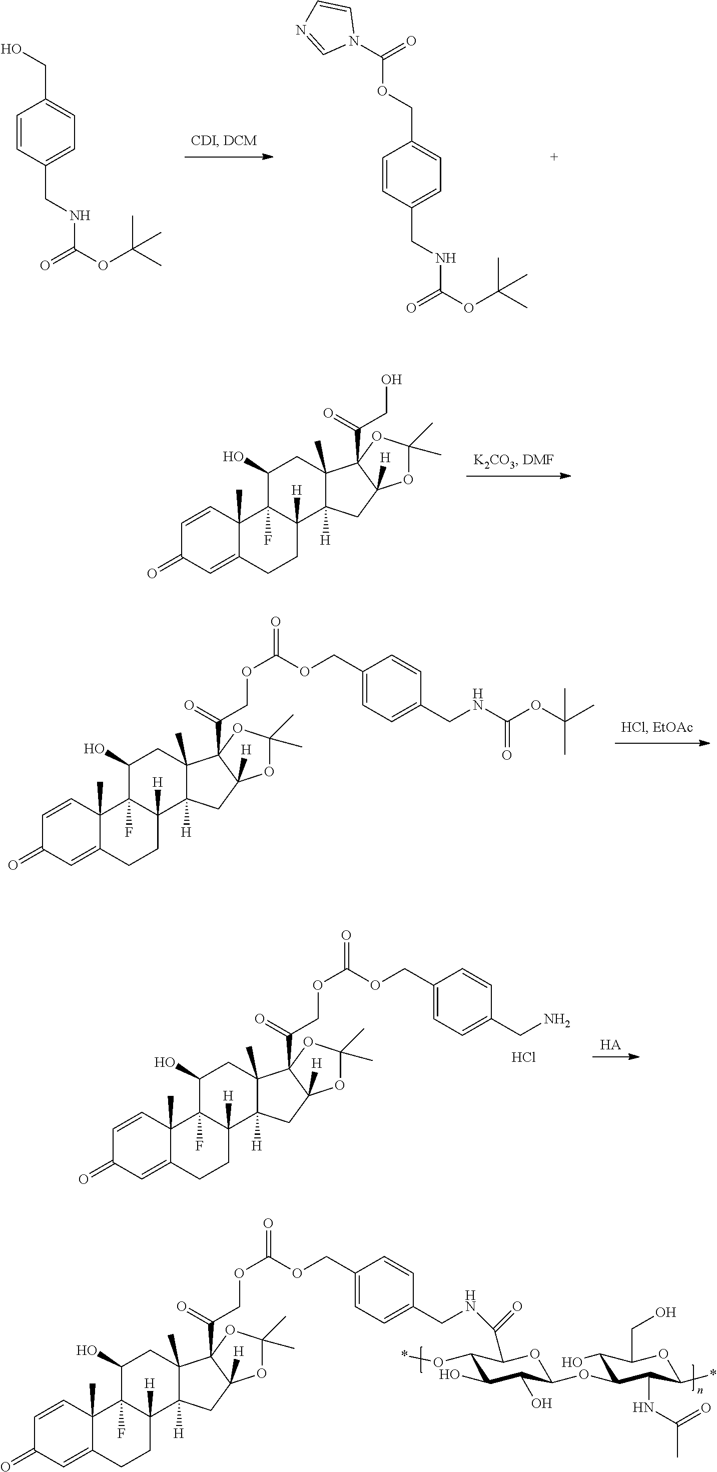

### Step 1: Preparation of 4-(((tert-butoxycarbonyl)amino)methyl)benzyl 16-imidazole-n-carboxylate

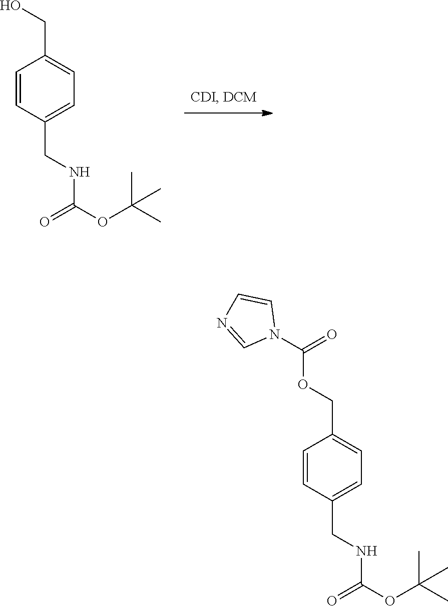 [SMILES: CC(C)(C)OC(=O)NCc1ccc(CO)cc1 ; CC(C)(C)OC(=O)NCc1ccc(COC(=O)n2ccnc2)cc1]

To a solution of tert-butyl (4-(hydroxymethyl)benzyl)carbamate (356 mg, 1.5 mmol) in DCM (10 mL) was added 1,1′-Carbonyldiimidazole (CDI, 486 mg, 3 mmol). The resulting mixture was stirred at room temperature for 16 hours. The solution was diluted with DCM and washed with saturated NaHCO₃ solution, water and saturated brine solution. The organic layer was dried over sodium sulfate, filtered and then concentrated under reduced pressure to afford the title compound as a yellow oil (0.49 g, yield: 98.6%) that was used without further purification.

### Step 2: Preparation of tert-butyl (4-((((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)methyl)benzyl)carbamate

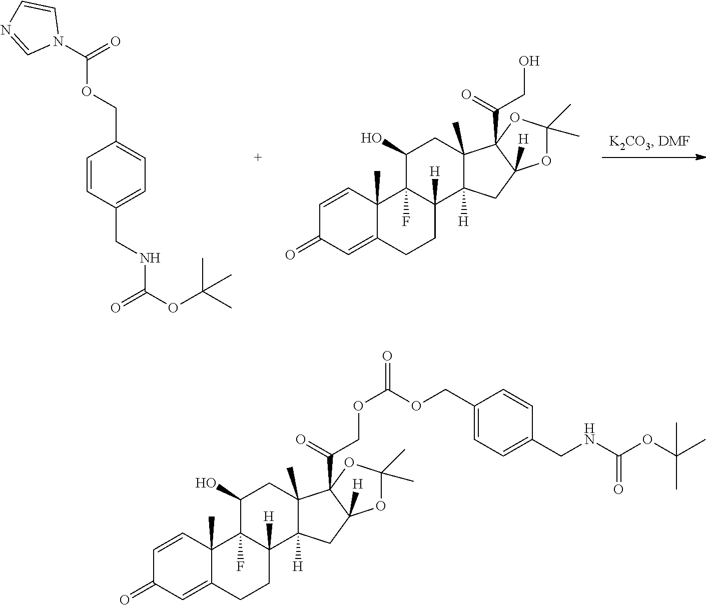

To a solution of 9α-Fluoro-11β3,16α,17,21-tetrahydroxy-1,4-pregnadiene-3,20-dione (Triamcinolone acetonide, 521 mg, 1.2 mmol) in DMF (10 mL) was added 4-(((tert-butoxycarbonyl)amino)methyl)benzyl 1H-imidazole-1-carboxylate (477 mg, 1.44 mmol) and K₂CO₃ (166 mg, 1.2 mmol) under N₂. The resulting mixture was stirred at room temperature for 16 hours. The reaction mixture was poured into water (30 mL) at room temperature, filtered and the filter cake was dissolved in DCM (40 mL). The solution was dried over sodium sulfate, filtered and then concentrated under reduced pressure. The residue was purified by silical gel chromatography to give the title product (0.42 g, yield: 50.2%); MS (m/z): [M+H]⁺ calcd for C₃₈H₄₈FNO₁₀, 698.80; found, 642.0 (M+H−56). ¹H NMR (400 MHz, Chloroform-d) δ 7.35 (d, J=8.0 Hz, 2H), 7.29 (d, J=8.0 Hz, 2H), 7.18 (d, J=10.1 Hz, 1H), 6.34 (dd, J=10.1, 1.9 Hz, 1H), 6.13 (t, J=1.7 Hz, 1H), 5.18 (s, 2H), 5.12-4.98 (m, 2H), 4.89 (s, 1H), 4.79 (d, J=17.8 Hz, 1H), 4.41 (dt, J=9.1, 2.9 Hz, 1H), 4.32 (d, J=6.0 Hz, 2H), 2.71-2.57 (m, 1H), 2.53-2.34 (m, 3H), 2.10 (dt, J=12.2, 6.2 Hz, 1H), 1.94-1.81 (m, 2H), 1.72-1.59 (m, 4H), 1.54 (s, 3H), 1.46 (s, 9H), 1.42 (s, 3H), 1.21 (s, 3H), 0.94 (s, 3H).

### Step 3: Preparation of 4-(aminomethyl)benzyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride

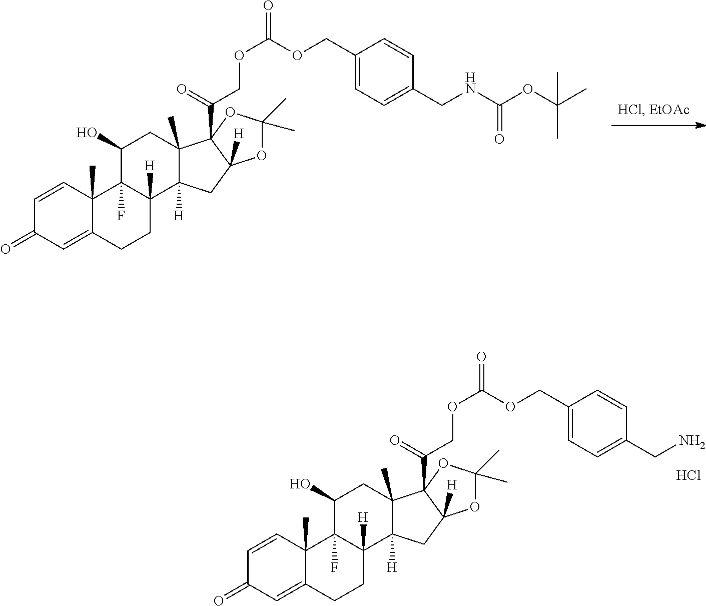

To a stirred solution of tert-butyl (4-((((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)methyl)benzyl)carbamate (418 mg, 0.6 mmol) in EtOAc (20 mL) was slowly added 4M HCl in ethyl acetate (commercially available) (4 mL) at ice-bath. The reaction mixture was allowed to stir at room temperature for 40 hours.

The solution was diluted with EtOAc and concentrated under reduced pressure to afford the title compound as a white solid (0.3 g, yield: 83.6%) that was used without further purification. MS (m/z): [M+H]⁺ calcd for C₃₃H₄₀FNO₈, 598.68; found, 598.3.

### Step 4: Preparation of Conjugate of HA and 4-(aminomethyl)benzyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

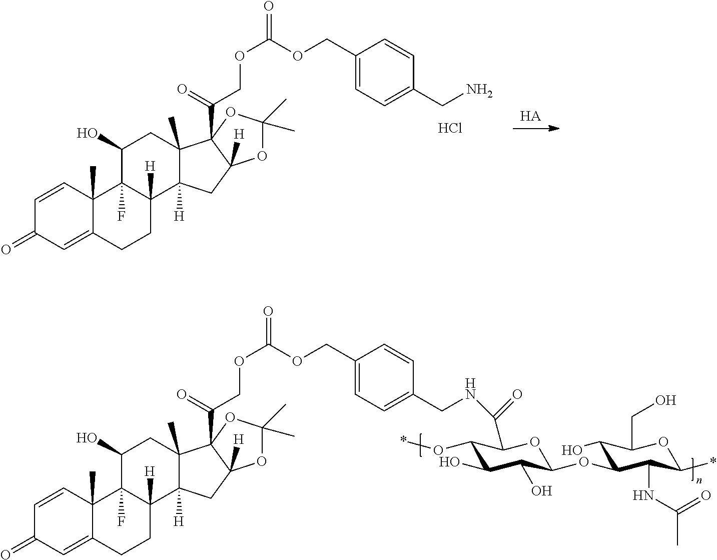

Hyaluronic acid (201 mg, 0.5 mmol carboxylic acid) was dissolved in 22 mL of deionized water in a 100 mL round-bottomed flask followed by the dropwise addition of 22 mL of 1,4-Dioxane while stirring. To the solution was added 4-(aminomethyl)benzyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride (127 mg, 0.2 mmol), N-Hydroxysuccinimide (NHS, 46 mg, 0.4 mmol) and 1-ethyl-3-(3-dimethylaminopropyl)carbodiimide (EDCI, 38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (5 mL) was added to the reaction mixture, after stirring for 1 hour, 1M HCl solution (4.3 mL) was added. NaCl (293 mg, 5 mmol) was then added to the reaction mixture, which was stirred for 1 hour and followed by the dropwise addition of anhydrous alcohol (200 mL) while stirring at −10° C. The mixture was filtered. The filter cake was collected, washed with anhydrous alcohol and dried in vacuo to give the title compound as a white solid. (sodium hyaluronate MW 50 KDa 0.21 g, yield: 47.7%, DSR=18%); ¹H NMR (400 MHz, Deuterium Oxide) δ 7.66-7.39 (m, 0.9H), 6.59-6.46 (m, 0.18H), 6.37-6.25 (m, 0.18H), 5.47-5.24 (m, 0.54H), 5.16-5.01 (m, 0.36H), 4.81-4.31 (m, 2.54H), 4.19-3.13 (m, 10H), 2.93-2.49 (m, 0.72H), 2.33-1.73 (m, 4.08H), 1.70-1.43 (m, 1.08H), 1.36-1.28 (m, 0.54H), 1.04-0.87 (m, 0.54H).

With this procedure, reaction of sodium hyaluronate (MW 500 KDa) provided corresponding product (0.19 g, yield: 43.1%, DSR=18%). ¹H NMR (400 MHz, Deuterium Oxide) δ 7.69-7.38 (m, 0.9H), 6.56-6.45 (m, 0.18H), 6.35-6.24 (m, 0.18H), 5.46-5.25 (m, 0.54H), 5.17-4.99 (m, 0.36H), 4.80-4.35 (m, 2.54H), 4.20-3.06 (m, 10H), 2.91-2.50 (m, 0.72H), 2.35-1.72 (m, 4.08H), 1.70-1.44 (m, 1.08H), 1.36-1.27 (m, 0.54H), 1.04-0.89 (m, 0.54H).

With this procedure, reaction of sodium hyaluronate (MW 2000 KDa) provided corresponding product (0.2 g, yield: 45.4%, DSR=15%). ¹H NMR (400 MHz, Deuterium Oxide) δ 7.70-7.43 (m, 0.75H), 6.63-6.50 (m, 0.15H), 6.41-6.32 (m, 0.15H), 5.50-5.31 (m, 0.45H), 5.22-5.03 (m, 0.3H), 4.82-4.40 (m, 2.45H), 4.25-3.09 (m, 10H), 2.94-2.52 (m, 0.6H), 2.38-1.76 (m, 3.9H), 1.72-1.51 (m, 0.9H), 1.42-1.32 (m, 0.45H), 1.07-0.90 (m, 0.45H).

### Example 16

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl pyrrolidin-3-yl carbonate

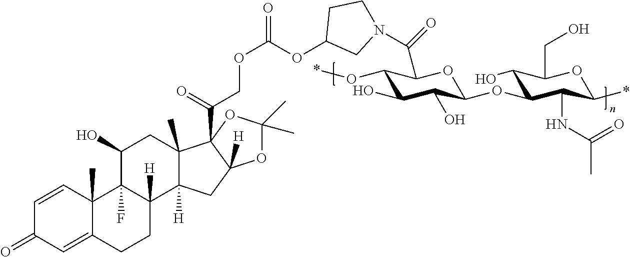

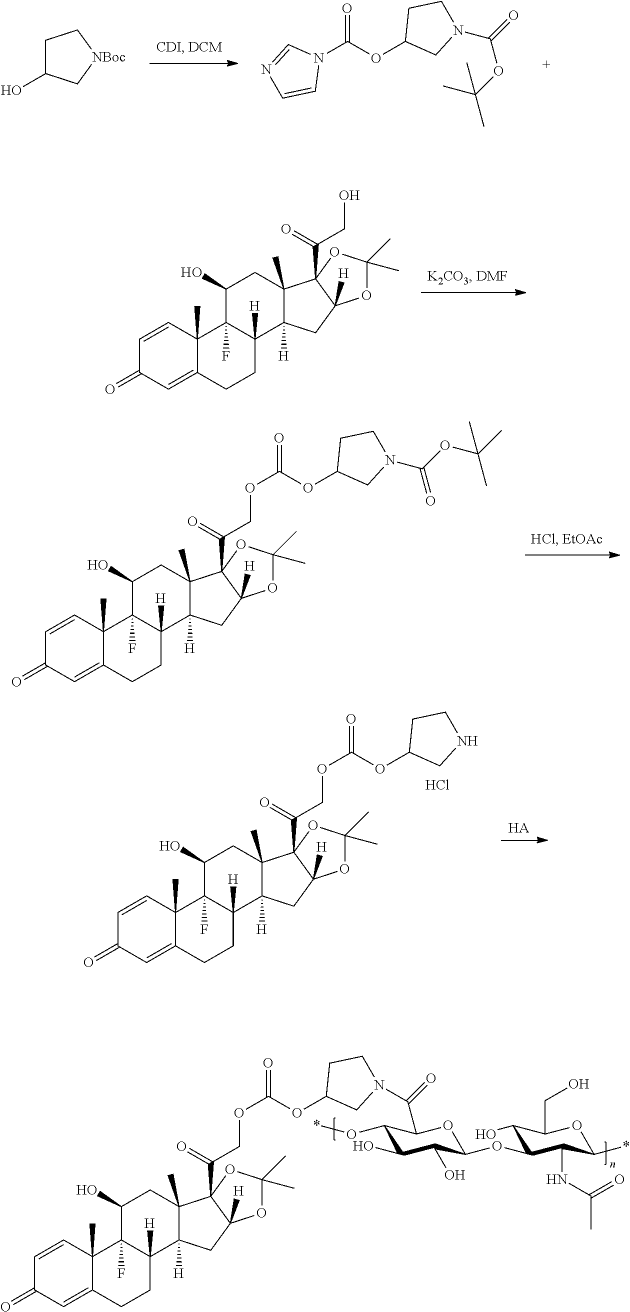

### Step 1: Preparation of 1-(tert-butoxycarbonyl)pyrrolidin-3-yl 1H-imidazole-1-carboxylate

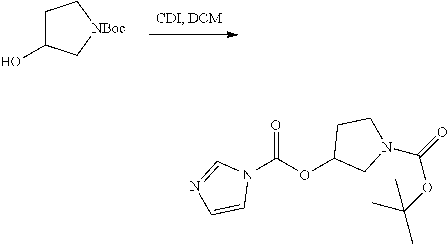 [SMILES: CC(C)(C)OC(=O)N1CCC(O)C1 ; CC(C)(C)OC(=O)N1CCC(OC(=O)n2ccnc2)C1]

To a solution of tert-butyl 3-hydroxypyrrolidine-1-carboxylate (281 mg, 1.5 mmol) in DCM (10 mL) was added 1,1′-Carbonyldiimidazole (CDI, 486 mg, 3 mmol). The resulting mixture was stirred at room temperature for 16 hours. The solution was diluted with DCM and washed with saturated NaHCO₃ solution, water and saturated brine solution. The organic layer was dried over sodium sulfate, filtered and then concentrated under reduced pressure to afford the title compound as a yellow oil (0.35 g, yield: 83%) that was used without further purification. MS (m/z): [M+H]⁺ calcd for C₁₃H₁₉N₃O₄, 282.31; found, 282.1.

### Step 2: Preparation of tert-butyl 3-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)pyrrolidine-1-carboxylate

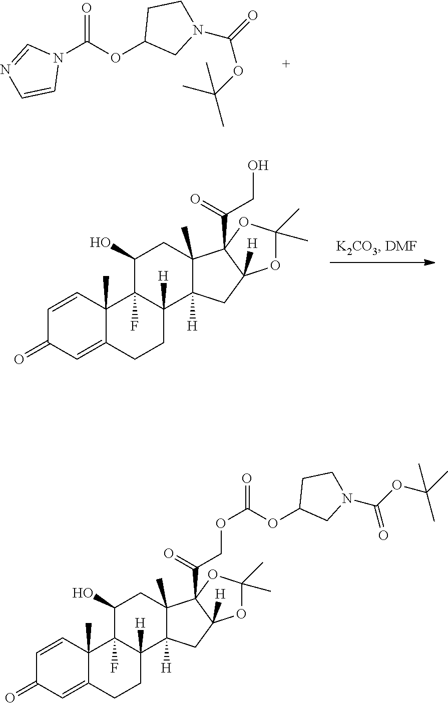

To a solution of 9α-Fluoro-11β,16α,17,21-tetrahydroxy-1,4-pregnadiene-3,20-dione (Triamcinolone acetonide, 564 mg, 1.3 mmol) in DMF (10 mL) was added 1-(tert-butoxycarbonyl)pyrrolidin-3-yl 1H-imidazole-1-carboxylate (365 mg, 1.3 mmol) and K₂CO₃ (179 mg, 1.3 mmol) under N₂. The resulting mixture was stirred at room temperature for 16 hours. The reaction mixture was poured into water (30 mL) at room temperature, filtered and the filter cake was dissolved in DCM (40 mL). The solution was dried over sodium sulfate, filtered and then concentrated under reduced pressure. The residue was purified by silical gel chromatography to give the title product (0.7 g, yield: 50.2%); MS (m/z): [M+H]⁺ calcd for C₃₄H₄₆FNO₁₀, 648.74; found, 592.2 (M+H−56). ¹H NMR (400 MHz, Chloroform-d) δ 7.24-7.18 (m, 1H), 6.35 (dd, J=10.1, 1.9 Hz, 1H), 6.13 (t, J=1.8 Hz, 1H), 5.21 (t, J=4.1 Hz, 1H), 5.13 (d, J=17.8 Hz, 1H), 5.00 (dd, J=5.2, 2.2 Hz, 1H), 4.90-4.69 (m, 1H), 4.48-4.35 (m, 1H), 3.53 (d, J=29.5 Hz, 4H), 2.70-2.30 (m, 5H), 2.26-2.06 (m, 3H), 1.87 (dt, J=11.8, 5.4 Hz, 1H), 1.76-1.52 (m, 7H), 1.47 (s, 9H), 1.43 (s, 3H), 1.21 (d, J=6.8 Hz, 3H), 0.95 (d, J=6.5 Hz, 3H).

### Step 3: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl pyrrolidin-3-yl carbonate hydrochloride

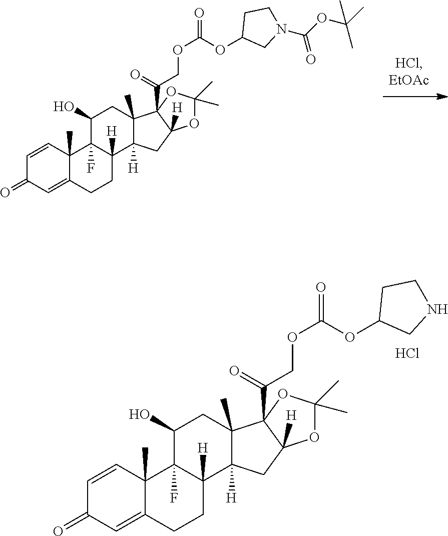

To a stirred solution of tert-butyl 3-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)pyrrolidine-1-carboxylate (421 mg, 0.65 mmol) in EtOAc (20 mL) was slowly added 4M HCl in ethyl acetate (commercially available) (4 mL) at ice-bath. The reaction mixture was allowed to stir at room temperature for 40 hours. The solution was diluted with EtOAc and concentrated under reduced pressure to afford the title compound as a white solid (0.3 g, yield: 79%) that was used without further purification. MS (m/z): [M+H]⁺ calcd for C₂₉H₃₈FNO₈, 548.62; found, 548.2.

### Step 4: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl pyrrolidin-3-yl carbonate

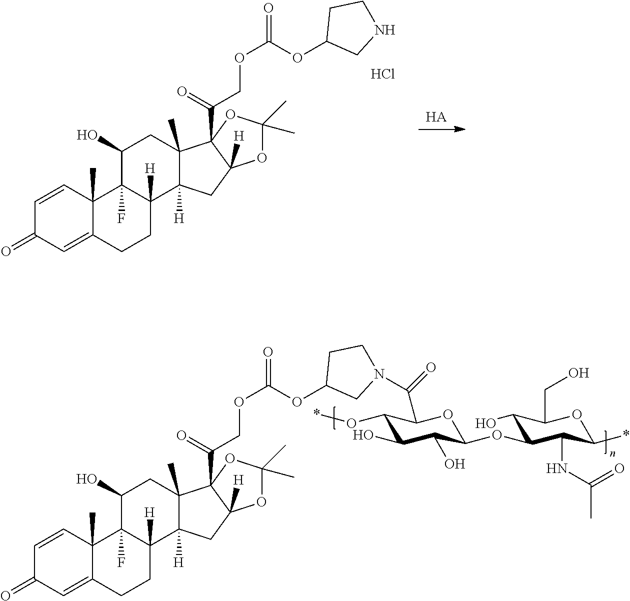

Hyaluronic acid (201 mg, 0.5 mmol carboxylic acid) was dissolved in 22 mL of deionized water in a 100 mL round-bottomed flask followed by the dropwise addition of 22 mL of 1,4-Dioxane while stirring. To the solution was added 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl pyrrolidin-3-yl carbonate hydrochloride (117 mg, 0.2 mmol), N-Hydroxysuccinimide (NHS, 46 mg, 0.4 mmol) and 1-ethyl-3-(3-dimethylaminopropyl)carbodiimide (EDCI, 38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (5 mL) was added to the reaction mixture, after stirring for 1 hour, 1M HCl solution (4.3 mL) was added. NaCl (293 mg, 5 mmol) was then added to the reaction mixture, which was stirred for 1 hour and followed by the dropwise addition of anhydrous alcohol (200 mL) while stirring at −10° C. The mixture was filtered. The filter cake was collected, washed with anhydrous alcohol and dried in vacuo to give the title compound as a white solid. (sodium hyaluronate MW 50 KDa 0.19 g, yield: 61%, DSR=9%); ¹H NMR (400 MHz, Deuterium Oxide) δ 7.66-7.57 (m, 0.09H), 6.57-6.49 (m, 0.09H), 6.38-6.29 (m, 0.09H), 5.54-5.30 (m, 0.27H), 5.18-5.03 (m, 0.09H), 4.82-4.38 (m, 2.09H), 4.23-3.20 (m, 10.36H), 2.96-2.50 (m, 0.36H), 2.45-1.72 (m, 3.36H), 1.70-1.46 (m, 0.9H), 1.40-1.27 (m, 0.27H), 1.06-0.89 (m, 0.27H).

With this procedure, reaction of sodium hyaluronate (MW 500 KDa) provided corresponding product (0.15 g, yield: 48.2%, DSR=10%). ¹H NMR (400 MHz, Deuterium Oxide) δ 7.65-7.56 (m, 0.1H), 6.57-6.48 (m, 0.1H), 6.38-6.28 (m, 0.1H), 5.52-5.30 (m, 0.3H), 5.22-5.04 (m, 0.1H), 4.81-4.37 (m, 2.1H), 4.27-3.10 (m, 10.4H), 2.93-2.51 (m, 0.4H), 2.43-1.72 (m, 3.4H), 1.69-1.46 (m, 1H), 1.39-1.27 (m, 0.3H), 1.06-0.88 (m, 0.3H).

With this procedure, reaction of sodium hyaluronate (MW 2000 KDa) provided corresponding product (0.16 g, yield: 51.4%, DSR=6%). ¹H NMR (400 MHz, Deuterium Oxide) δ 7.69-7.61 (m, 0.06H), 6.61-6.51 (m, 0.06H), 6.40-6.31 (m, 0.06H), 5.52-5.33 (m, 0.18H), 5.24-5.06 (m, 0.06H), 4.82-4.40 (m, 2.06H), 4.25-3.14 (m, 10.24H), 2.94-2.59 (m, 0.24H), 2.52-1.73 (m, 3.24H), 1.71-1.50 (m, 0.6H), 1.41-1.32 (m, 0.18H), 1.06-0.92 (m, 0.18H).

### Example 17

### Preparation of Conjugate of HA and 2-aminoethyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

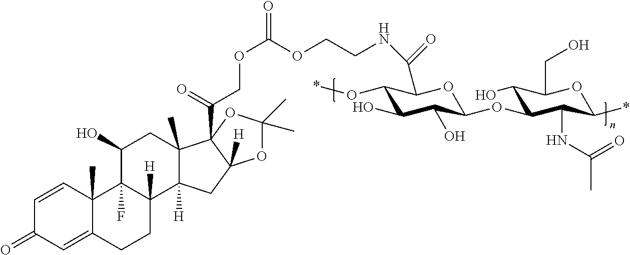

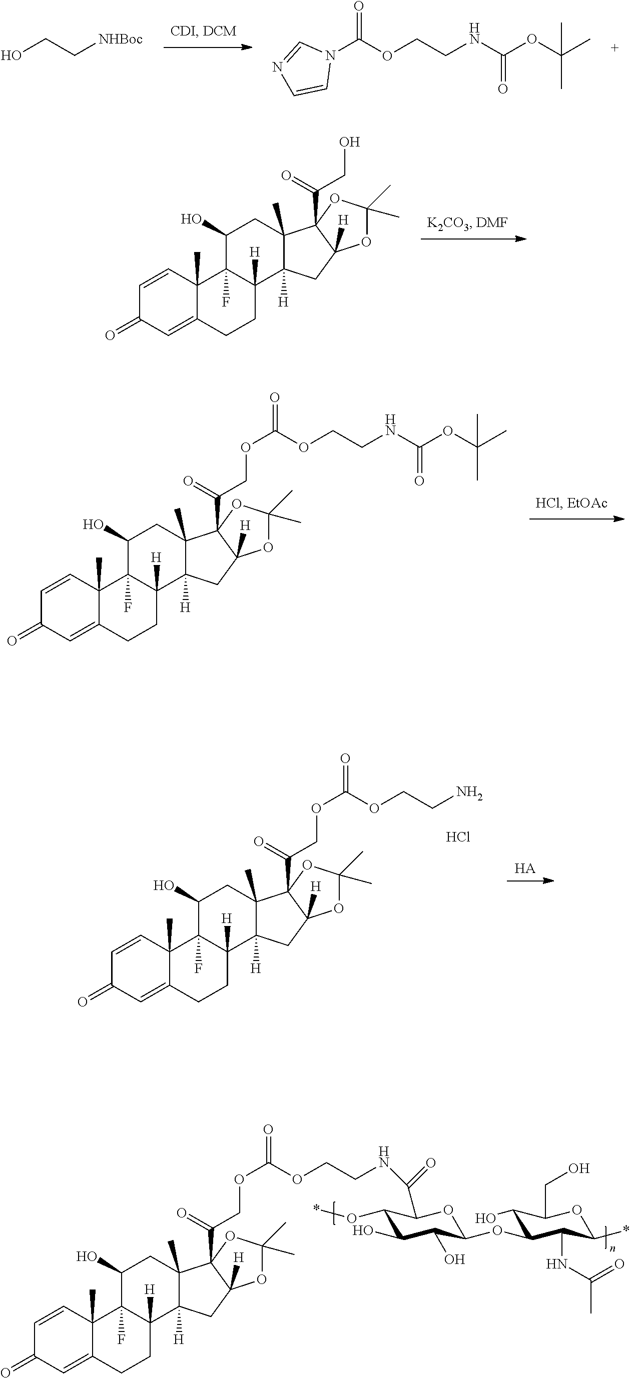

### Step 1: Preparation of 2-((tert-butoxycarbonyl)amino)ethyl 1H-imidazole-1-carboxylate

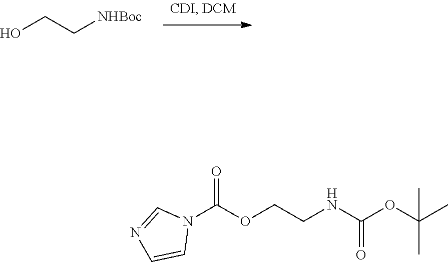 [SMILES: CC(C)(C)OC(=O)NCCO ; CC(C)(C)OC(=O)NCCOC(=O)n1ccnc1]

To a solution of tert-butyl (2-hydroxyethyl)carbamate (1600 mg, 10 mmol) in DCM (100 mL) was added 1,1′-Carbonyldiimidazole (CDI, 3200 mg, 20 mmol). The resulting mixture was stirred at room temperature for 16 hours. The solution was diluted with DCM and washed with saturated NaHCO₃ solution, water and saturated brine solution. The organic layer was dried over sodium sulfate, filtered and then concentrated under reduced pressure to afford the title compound as a yellow oil (2.5 g, yield: 98%) that was used without further purification. MS (m/z): [M+H]⁺ calcd for C₁₁H₁₇N₃O₄, 256.27; found, 256.2.

### Step 2: Preparation of tert-butyl (2-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)ethyl)carbamate

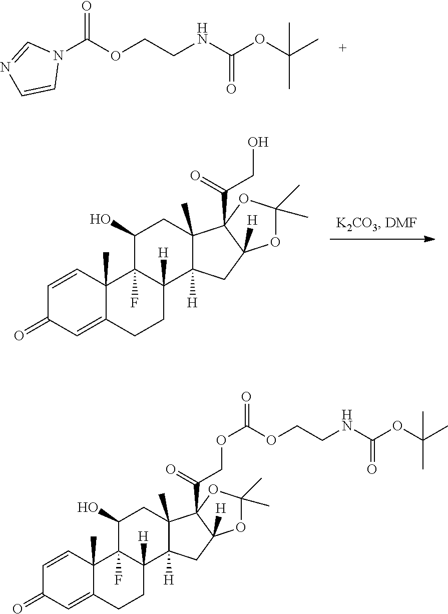

To a solution of 9α-Fluoro-11β,16α,17,21-tetrahydroxy-1,4-pregnadiene-3,20-dione (Triamcinolone acetonide, 434 mg, 1 mmol) in DMF (10 mL) was added 2-((tert-butoxycarbonyl)amino)ethyl 1H-imidazole-1-carboxylate (505 mg, 2 mmol) and K₂CO₃ (138 mg, 1 mmol) under N₂. The resulting mixture was stirred at room temperature for 16 hours. The reaction mixture was poured into water (30 mL) at room temperature, filtered and the filter cake was dissolved in DCM (40 mL). The solution was dried over sodium sulfate, filtered and then concentrated under reduced pressure. The residue was purified by silical gel chromatography to give the title product (0.26 g, yield: 41.8%); MS (m/z): [M+H]⁺ calcd for C₃₂H₄₄FNO₁₀, 622.70; found, 566.2 (M+H−56). ¹H NMR (400 MHz, Chloroform-d) δ 7.21 (d, J=10.2 Hz, 1H), 6.35 (dd, J=10.1, 1.9 Hz, 1H), 6.13 (d, J=1.8 Hz, 1H), 5.11-4.99 (m, 2H), 4.92 (s, 1H), 4.81 (d, J=17.8 Hz, 1H), 4.43 (dq, J=8.9, 2.7 Hz, 1H), 4.24 (q, J=5.8 Hz, 2H), 3.54-3.37 (m, 2H), 2.71-2.28 (m, 5H), 2.10 (td, J=12.5, 5.8 Hz, 1H), 1.87 (dt, J=12.0, 5.3 Hz, 1H), 1.74-1.60 (m, 4H), 1.55 (s, 3H), 1.45 (s, 9H), 1.43 (s, 3H), 1.21 (s, 3H), 0.95 (s, 3H).

### Step 3: Preparation of 2-aminoethyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride

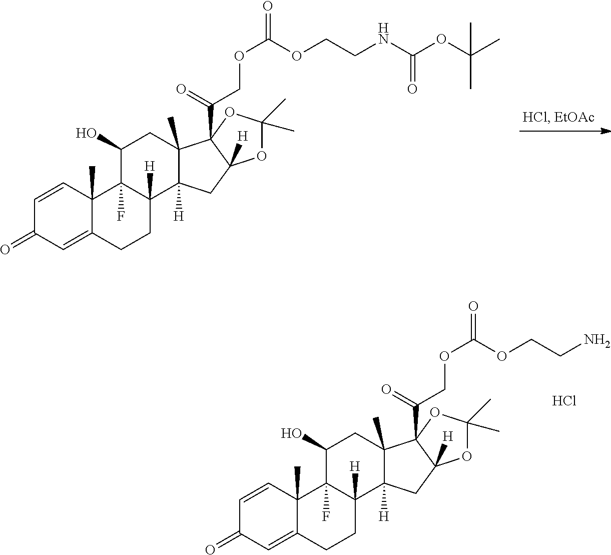

To a stirred solution of tert-butyl (2-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)ethyl)carbamate (249 mg, 0.4 mmol) in EtOAc (15 mL) was slowly added 4M HCl in ethyl acetate (3 mL) at ice-bath. The reaction mixture was allowed to stir at room temperature for 40 hours. The solution was diluted with EtOAc and concentrated under reduced pressure to afford the title compound as a white solid (0.22 g, yield: 98.6%) that was used without further purification. MS (m/z): [M+H]⁺ calcd for C₂₇H₃₆FNO₈, 522.58; found, 522.2.

### Step 3: Preparation of Conjugate of HA and 2-aminoethyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

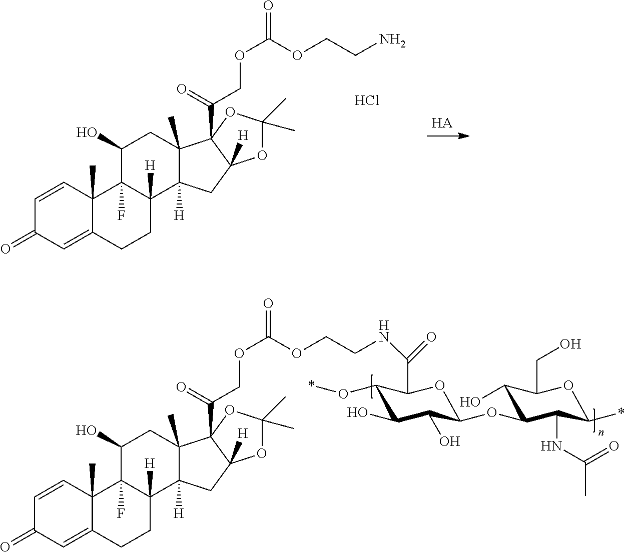

Hyaluronic acid (201 mg, 0.5 mmol carboxylic acid) was dissolved in 22 mL of deionized water in a 100 mL round-bottomed flask followed by the dropwise addition of 22 mL of 1,4-Dioxane while stirring. To the solution was added 2-aminoethyl (2-((6αS,6βR,7S,8αS,8S,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride (112 mg, 0.2 mmol), N-Hydroxysuccinimide (NHS, 46 mg, 0.4 mmol) and 1-ethyl-3-(3-dimethylaminopropyl)carbodiimide (EDCI, 38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (5 mL) was added to the reaction mixture, after stirring for 1 hour, 1M HCl solution (4.3 mL) was added. NaCl (293 mg, 5 mmol) was then added to the reaction mixture, which was stirred for 1 hour and followed by the dropwise addition of anhydrous alcohol (200 mL) while stirring at −10° C. The mixture was filtered. The filter cake was collected, washed with anhydrous alcohol and dried in vacuo to give the title compound as a white solid. (sodium hyaluronate MW 50 KDa 0.18 g, yield: 59%, DSR=14%); ¹H NMR (400 MHz, Deuterium Oxide) δ 7.68-7.59 (m, 0.14H), 6.58-6.50 (m, 0.14H), 6.37-6.28 (m, 0.14H), 5.42-5.29 (m, 0.14H), 5.24-5.09 (m, 0.28H), 4.80-4.34 (m, 2.42H), 4.26-3.20 (m, 10.28H), 2.96-2.51 (m, 0.7H), 2.35-1.73 (m, 3.28H), 1.70-1.49 (m, 1.26H), 1.43-1.31 (m, 0.42H), 1.08-0.94 (m, 0.42H).

With this procedure, reaction of sodium hyaluronate (MW 500 KDa) provided corresponding product (0.18 g, yield: 59%, DSR=16%). ¹H NMR (400 MHz, Deuterium Oxide) δ 7.69-7.59 (m, 0.16H), 6.60-6.51 (m, 0.16H), 6.40-6.30 (m, 0.16H), 5.50-5.32 (m, 0.16H), 5.24-5.01 (m, 0.32H), 4.81-4.38 (m, 2.48H), 4.23-3.15 (m, 10.32H), 2.94-2.49 (m, 0.8H), 2.37-1.74 (m, 3.32H), 1.71-1.50 (m, 1.44H), 1.43-1.32 (m, 0.48H), 1.09-0.93 (m, 0.48H).

With this procedure, reaction of sodium hyaluronate (MW 2000 KDa) provided corresponding product (0.19 g, yield: 62.2%, DSR=14%). ¹H NMR (400 MHz, Deuterium Oxide) δ 7.70-7.59 (m, 0.14H), 6.60-6.50 (m, 0.14H), 6.40-6.32 (m, 0.14H), 5.52-5.32 (m, 0.14H), 5.24-5.02 (m, 0.28H), 4.82-4.34 (m, 2.42H), 4.27-3.13 (m, 10.28H), 3.02-2.49 (m, 0.7H), 2.38-1.75 (m, 3.28H), 1.73-1.48 (m, 1.26H), 1.42-1.32 (m, 0.42H), 1.10-0.95 (m, 0.42H).

### Example 18

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 5-aminopentanoate

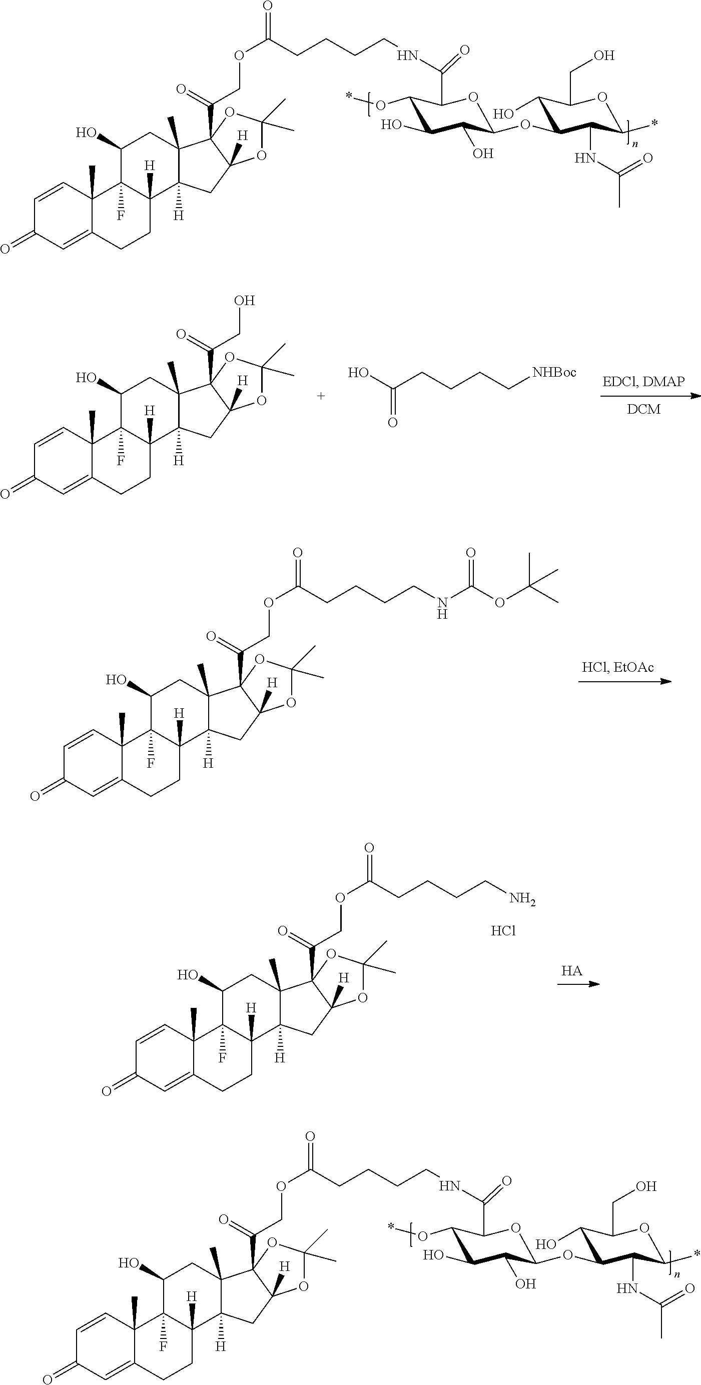

### Step 1: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 5-((tert-butoxycarbonyl)amino)pentanoate

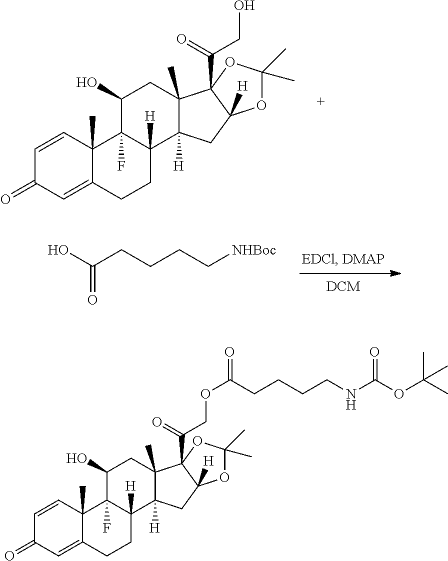

9α-Fluoro-11β,16α,17,21-tetrahydroxy-1,4-pregnadiene-3,20-dione (Triamcinolone acetonide, 435 mg, 1 mmol), 5-((tert-butoxycarbonyl)amino)pentanoic acid (282 mg, 1.3 mmol), 1-ethyl-3-(3-dimethylaminopropyl)carbodiimide (EDCI, 249 mg, 1.3 mmol), N,N-dimethylpyridin-4-amine (DMAP, 12 mg, 0.1 mmol) were dissolved in DCM (20 mL). The reaction mixture was stirred at room temperature for 24 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=5:1˜2:1) to afford the title compound (600 mg, Yield: 94.7%). MS (m/z): [M+H]⁺ calcd for C₃₄H₄₈FNO₉, 634.75; found, 578.3 (M+H−56). ¹H NMR (400 MHz, Chloroform-d) δ 7.21 (d, J=10.1 Hz, 1H), 6.35 (dd, J=10.1, 1.9 Hz, 1H), 6.13 (t, J=1.7 Hz, 1H), 4.98 (d, J=4.9 Hz, 1H), 4.90 (d, J=2.4 Hz, 2H), 4.63 (t, J=5.8 Hz, 1H), 4.42 (d, J=8.7 Hz, 1H), 3.14 (q, J=6.7 Hz, 2H), 2.69-2.58 (m, 1H), 2.55-2.43 (m, 3H), 2.43-2.32 (m, 3H), 2.28-2.18 (m, 1H), 2.11 (td, J=12.5, 5.9 Hz, 1H), 1.87 (dt, J=11.9, 5.3 Hz, 1H), 1.80-1.53 (m, 10H), 1.49-1.39 (m, 12H), 1.21 (s, 3H), 0.94 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 5-aminopentanoate hydrochloride

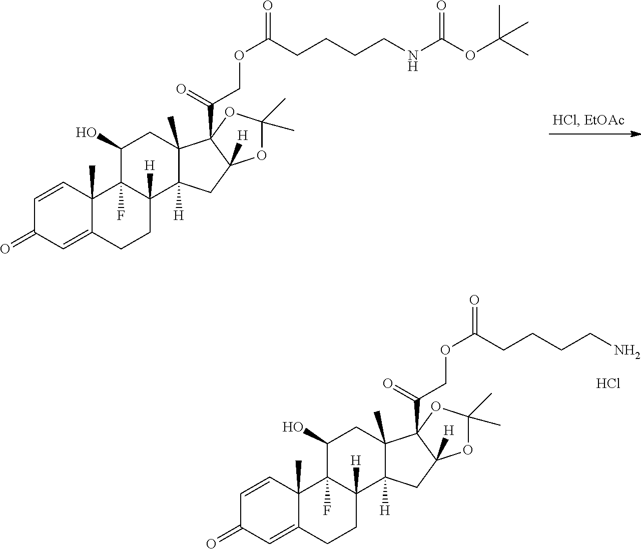

To a stirred solution of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 5-((tert-butoxycarbonyl)amino)pentanoate (600 mg, 0.95 mmol) in EtOAc (24 mL) was slowly added 4M HCl in ethyl acetate (4.8 mL) at ice-bath. The reaction mixture was allowed to stir at room temperature for 18 hours. The solution was diluted with EtOAc and concentrated under reduced pressure to afford the title compound as a white solid (0.51 g, yield: 94.2%) that was used without further purification. MS (m/z): [M+H]⁺ calcd for C₂₉H₄₀FNO₇, 534.64; found, 534.3.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 5-aminopentanoate

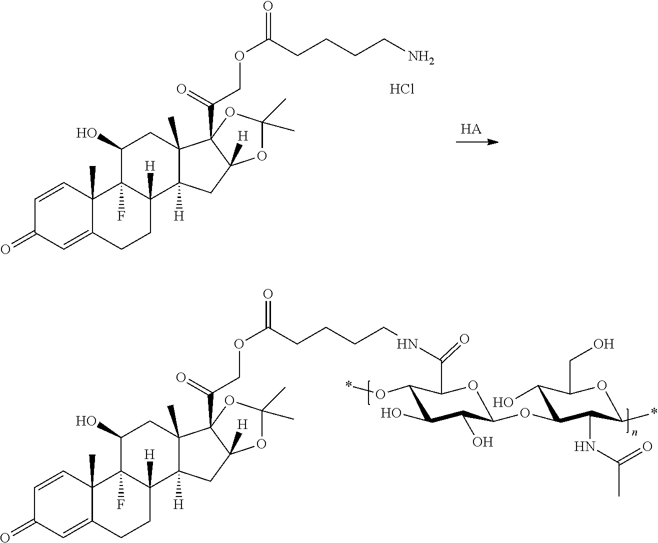

Hyaluronic acid (201 mg, 0.5 mmol carboxylic acid) was dissolved in 22 mL of deionized water in a 100 mL round-bottomed flask followed by the dropwise addition of 22 mL of 1,4-Dioxane while stirring. To the solution was added 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 5-aminopentanoate hydrochloride (114 mg, 0.2 mmol), N-Hydroxysuccinimide (NHS, 46 mg, 0.4 mmol) and 1-ethyl-3-(3-dimethylaminopropyl)carbodiimide (EDCI, 38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (5 mL) was added to the reaction mixture, after stirring for 1 hour, 1M HCl solution (4.3 mL) was added. NaCl (293 mg, 5 mmol) was then added to the reaction mixture, which was stirred for 1 hour and followed by the dropwise addition of anhydrous alcohol (200 mL) while stirring at −10° C. The mixture was filtered. The filter cake was collected, washed with anhydrous alcohol and dried in vacuo to give the title compound as a white solid. (sodium hyaluronate MW 50 KDa 0.17 g, yield: 55.1%, DSR=10%); ¹H NMR (400 MHz, Deuterium Oxide) δ 7.65-7.56 (m, 0.1H), 6.56-6.47 (m, 0.1H), 6.36-6.27 (m, 0.1H), 5.37-5.26 (m, 0.1H), 5.19-5.01 (m, 0.2H), 4.80-4.38 (m, 2.1H), 4.29-3.09 (m, 10.2H), 2.89-2.46 (m, 0.5H), 2.37-1.67 (m, 3.3H), 1.66-1.49 (m, 1.4H), 1.40-1.30 (m, 0.3H), 1.05-0.90 (m, 0.3H).

With this procedure, reaction of sodium hyaluronate (MW 500 KDa) provided corresponding product (0.19 g, yield: 61.6%, DSR=16%). ¹H NMR (400 MHz, Deuterium Oxide) δ 7.67-7.57 (m, 0.16H), 6.57-6.49 (m, 0.16H), 6.37-6.28 (m, 0.16H), 5.40-5.28 (m, 0.16H), 5.20-5.02 (m, 0.32H), 4.79-4.40 (m, 2.16H), 4.29-3.08 (m, 10.32H), 2.94-2.51 (m, 0.8H), 2.39-1.68 (m, 3.48H), 1.66-1.50 (m, 2.24H), 1.42-1.30 (m, 0.48H), 1.06-0.93 (m, 0.48H).

With this procedure, reaction of sodium hyaluronate (MW 500 KDa) provided corresponding product (0.17 g, yield: 55.1%, DSR=8%). ¹H NMR (400 MHz, Deuterium Oxide) δ 7.67-7.55 (m, 0.08H), 6.56-6.46 (m, 0.08H), 6.36-6.26 (m, 0.08H), 5.38-5.27 (m, 0.08H), 5.20-5.01 (m, 0.16H), 4.79-4.36 (m, 2.08H), 4.27-3.04 (m, 10.16H), 2.89-2.43 (m, 0.4H), 2.35-1.68 (m, 3.24H), 1.67-1.45 (m, 1.12H), 1.40-1.27 (m, 0.24H), 1.05-0.90 (m, 0.24H).

### Example 19

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl glycylglycylglycinate

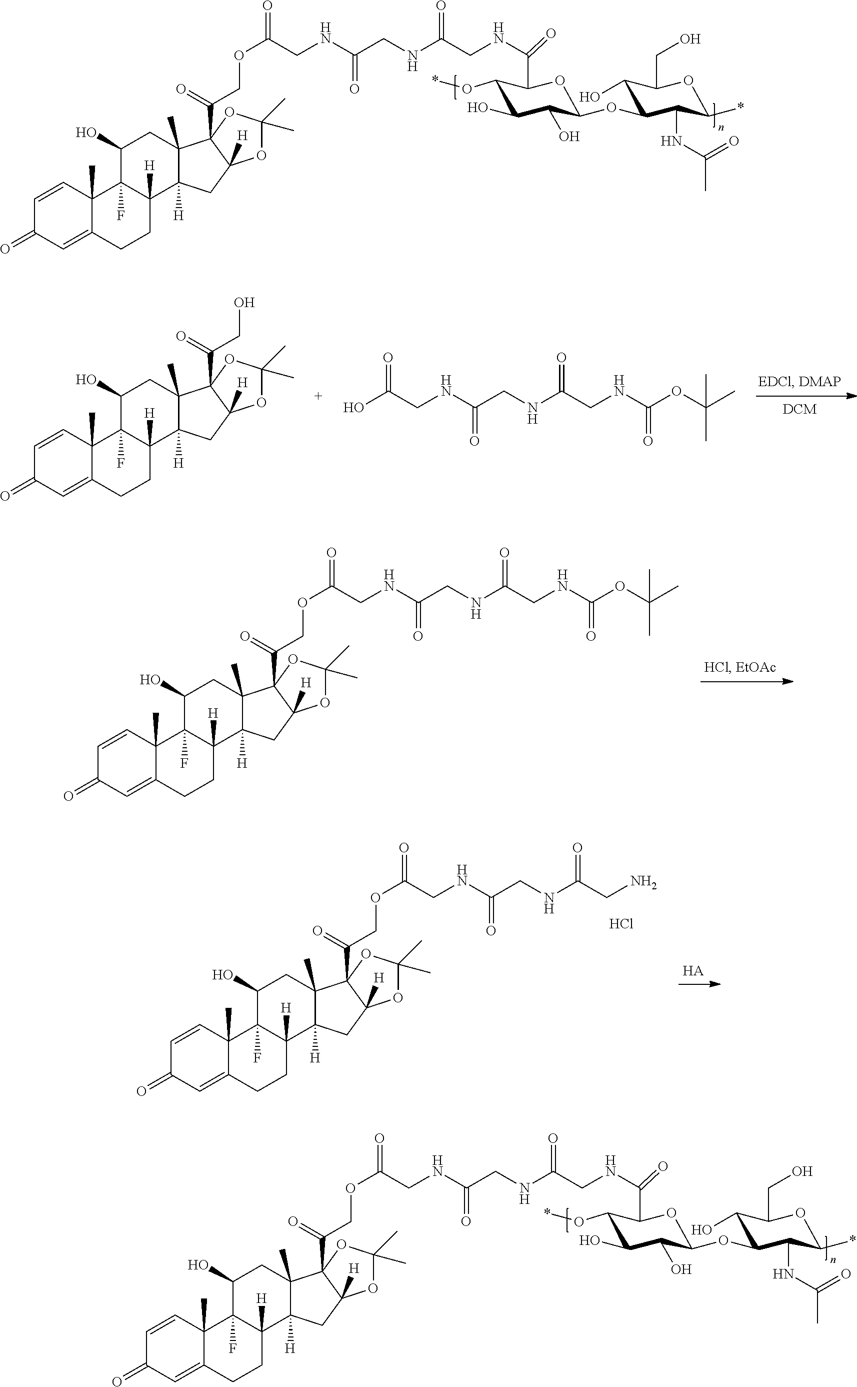

### Step 1: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (tert-butoxycarbonyl)glycylglycylglycinate

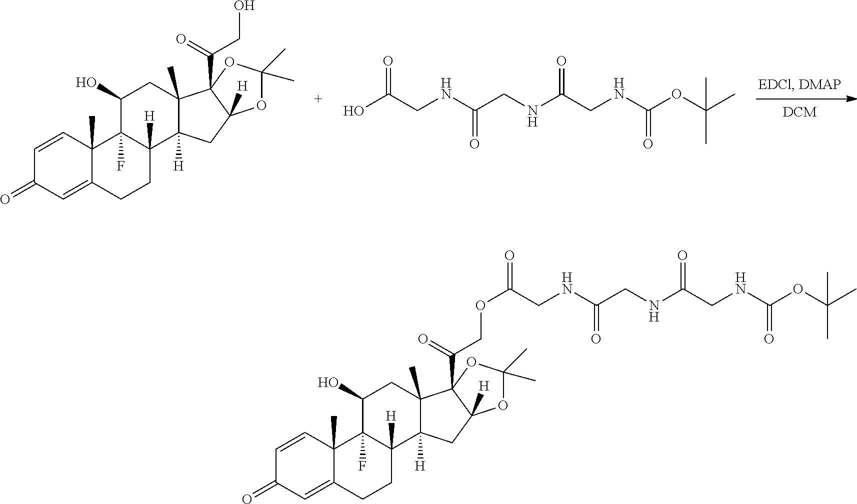

9α-Fluoro-11β,16α,17,21-tetrahydroxy-1,4-pregnadiene-3,20-dione (Triamcinolone acetonide, 435 mg, 1 mmol), (tert-butoxycarbonyl)glycylglycylglycine (376 mg, 1.3 mmol), 1-ethyl-3-(3-dimethylaminopropyl)carbodiimide (EDCI, 249 mg, 1.3 mmol), N,N-dimethylpyridin-4-amine (DMAP, 12 mg, 0.1 mmol) were dissolved in DCM (20 mL). The reaction mixture was stirred at room temperature for 4 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=5:1˜2:1) to afford the title compound (690 mg, Yield: 97.8%). MS (m/z): [M+H]⁺ calcd for C₃₅H₄₈FN₃O₁₁, 706.78; found, 706.2. ¹H NMR (400 MHz, Chloroform-d) δ 7.57-7.37 (m, 2H), 6.34 (dd, J=10.1, 1.9 Hz, 1H), 6.13 (t, J=1.7 Hz, 1H), 5.61 (s, 1H), 5.06-4.93 (m, 3H), 4.43 (dt, J=9.3, 2.9 Hz, 1H), 4.39-4.19 (m, 2H), 3.96 (ddt, J=41.3, 22.8, 10.7 Hz, 4H), 3.79-3.65 (m, 1H), 2.63 (td, J=13.6, 5.8 Hz, 1H), 2.54-2.34 (m, 2H), 2.29 (dt, J=14.0, 3.4 Hz, 1H), 2.15-2.01 (m, 2H), 1.87 (dd, J=12.5, 5.8 Hz, 1H), 1.77-1.53 (m, 7H), 1.44 (d, J=9.4 Hz, 12H), 1.20 (s, 3H), 0.89 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl glycylglycylglycinate hydrochloride

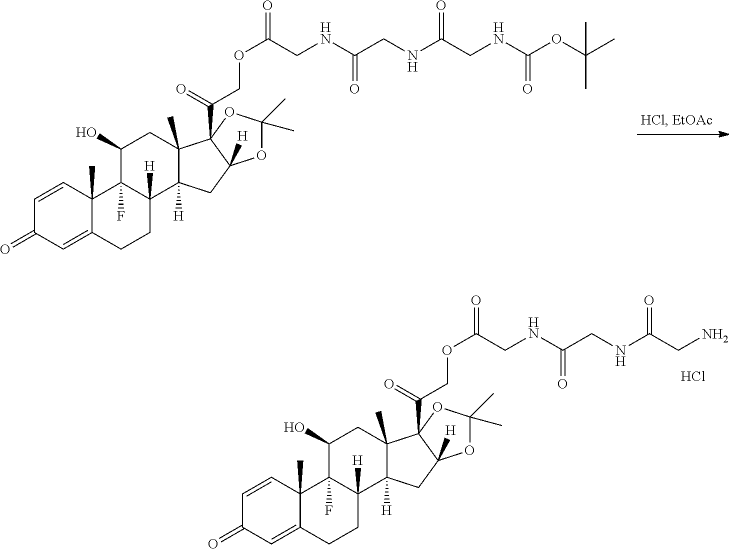

To a stirred solution of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (tert-butoxycarbonyl)glycylglycylglycinate (600 mg, 0.85 mmol) in EtOAc (24 mL) was slowly added 4M HCl in ethyl acetate (4.8 mL) at ice-bath. The reaction mixture was allowed to stir at room temperature for 18 hours. The solution was diluted with EtOAc and concentrated under reduced pressure to afford the title compound as a white solid (0.5 g, yield: 91.7%) that was used without further purification. MS (m/z): [M+H]⁺ calcd for C₃₀H₄₀FN₃O₉, 606.66; found, 606.3.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl glycylglycylglycinate

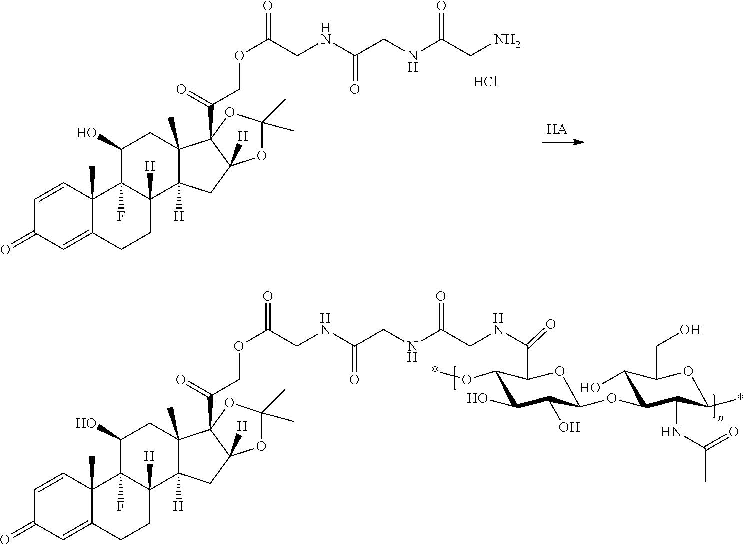

Hyaluronic acid (201 mg, 0.5 mmol carboxylic acid) was dissolved in 22 mL of deionized water in a 100 mL round-bottomed flask followed by the dropwise addition of 22 mL of 1,4-Dioxane while stirring. To the solution was added 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl glycylglycylglycinate hydrochloride (128 mg, 0.2 mmol), N-Hydroxysuccinimide (NHS, 46 mg, 0.4 mmol) and 1-ethyl-3-(3-dimethylaminopropyl)carbodiimide (EDCI, 38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (5 mL) was added to the reaction mixture, after stirring for 1 hour, 1M HCl solution (4.3 mL) was added. NaCl (1000 mg, 17 mmol) was then added to the reaction mixture, which was stirred for 1 hour and followed by the dropwise addition of anhydrous alcohol (200 mL) while stirring at −10° C. The mixture was filtered. The filter cake was collected, washed with anhydrous alcohol and dried in vacuo to give the title compound as a white solid. (sodium hyaluronate MW 50 KDa 0.18 g, yield: 55.9%, DSR=13%); ¹H NMR (400 MHz, Deuterium Oxide) δ 7.66-7.56 (m, 0.13H), 6.56-6.45 (m, 0.13H), 6.35-6.27 (m, 0.13H), 5.43-5.25 (m, 0.13H), 5.22-4.98 (m, 0.39H), 4.81-4.41 (m, 2.13H), 4.35-3.27 (m, 10.65H), 2.90-2.51 (m, 0.52H), 2.36-1.71 (m, 3.39H), 1.69-1.46 (m, 1.17H), 1.39-1.30 (m, 0.39H), 1.07-0.90 (m, 0.39H).

With this procedure, reaction of sodium hyaluronate (MW 500 KDa) provided corresponding product (0.2 g, yield: 62.1%, DSR=16%). ¹H NMR (400 MHz, Deuterium Oxide) δ 7.69-7.58 (m, 0.16H), 6.60-6.49 (m, 0.16H), 6.39-6.29 (m, 0.16H), 5.50-5.28 (m, 0.16H), 5.24-5.08 (m, 0.48H), 4.80-4.48 (m, 2.16H), 4.39-3.20 (m, 10.8H), 2.98-2.55 (m, 0.64H), 2.40-1.73 (m, 3.48H), 1.71-1.49 (m, 1.44H), 1.42-1.31 (m, 0.48H), 1.09-0.93 (m, 0.48H).

With this procedure, reaction of sodium hyaluronate (MW 2000 KDa) provided corresponding product (0.2 g, yield: 62.1%, DSR=13%). ¹H NMR (400 MHz, Deuterium Oxide) δ 7.70-7.59 (m, 0.13H), 6.61-6.50 (m, 0.13H), 6.41-6.31 (m, 0.13H), 5.51-5.28 (m, 0.13H), 5.25-5.08 (m, 0.39H), 4.82-4.44 (m, 2.13H), 4.37-3.15 (m, 10.65H), 2.96-2.54 (m, 0.52H), 2.40-1.75 (m, 3.39H), 1.72-1.48 (m, 1.17H), 1.43-1.33 (m, 0.39H), 1.10-0.92 (m, 0.39H).

### Example 20

### Preparation of Conjugate of HA and 1-aminopropan-2-yl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

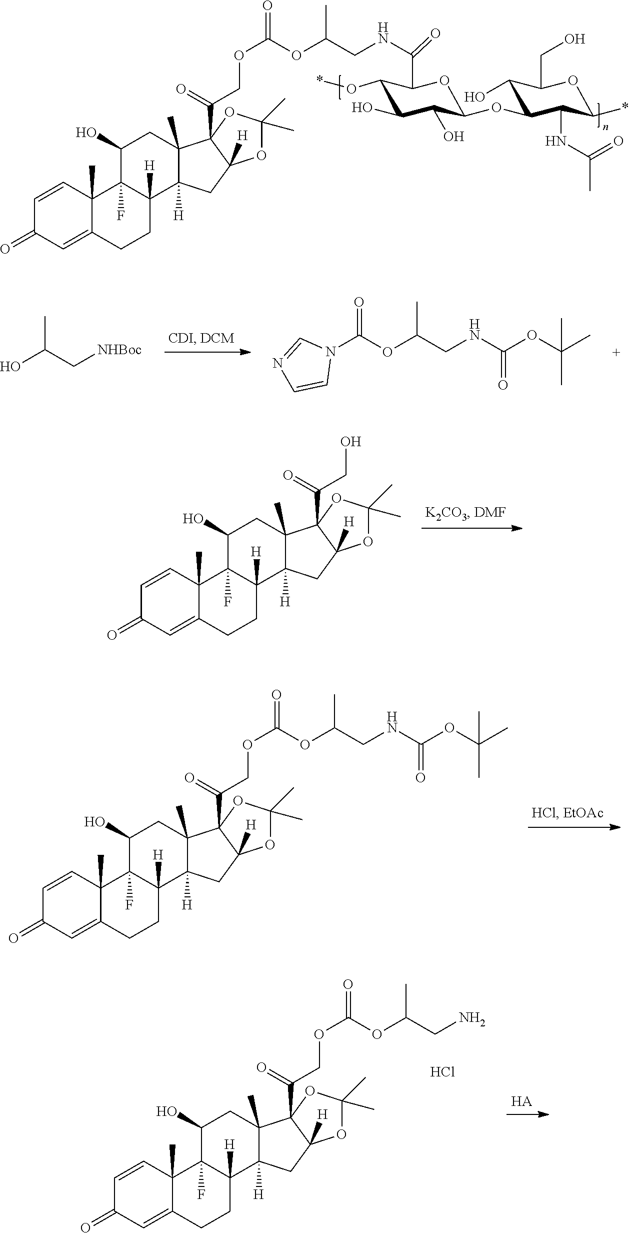

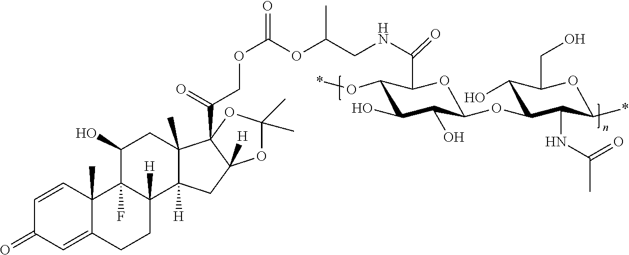

### Step 1: Preparation of 1-((tert-butoxycarbonyl)amino)propan-2-yl 1H-imidazole-1-carboxylate

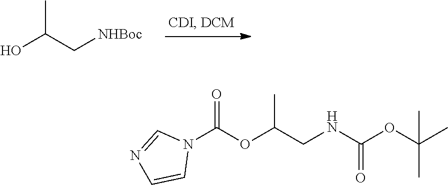 [SMILES: CC(O)CNC(=O)OC(C)(C)C ; CC(CNC(=O)OC(C)(C)C)OC(=O)n1ccnc1]

To a solution of tert-butyl (2-hydroxypropyl)carbamate (525 mg, 3 mmol) in DCM (30 mL) was added 1,1′-Carbonyldiimidazole (CDI, 972 mg, 6 mmol). The resulting mixture was stirred at room temperature for 16 hours. The solution was diluted with DCM and washed with saturated NaHCO₃ solution, water and saturated brine solution. The organic layer was dried over sodium sulfate, filtered and then concentrated under reduced pressure to afford the title compound as a yellow oil (0.8 g, yield: 98.7%) that was used without further purification. MS (m/z): [M+H]+ calcd for C₁₂H₁₉N₃O₄, 270.14; found, 270.2.

### Step 2: Preparation of tert-butyl (2-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)propyl)carbamate

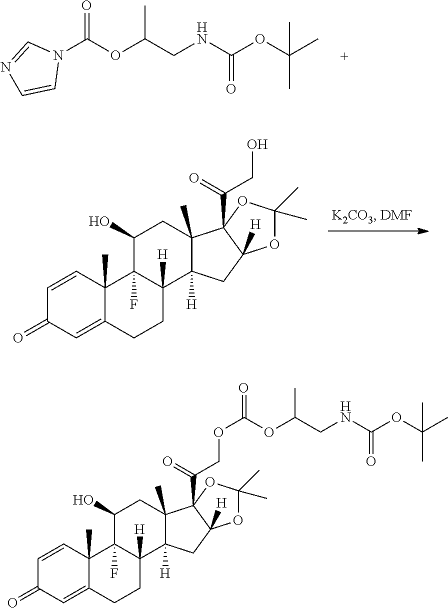

To a solution of 9α-Fluoro-11β,16α,17,21-tetrahydroxy-1,4-pregnadiene-3,20-dione (Triamcinolone acetonide, 1041 mg, 2.4 mmol) in DMF (30 mL) was added 1-((tert-butoxycarbonyl)amino)propan-2-yl 1H-imidazole-1-carboxylate (800 mg, 3 mmol) and K₂CO₃ (414 mg, 13 mmol) under N₂. The resulting mixture was stirred at room temperature for 16 hours. The reaction mixture was poured into water (90 mL) at room temperature, filtered and the filter cake was dissolved in DCM (40 mL). The solution was dried over sodium sulfate, filtered and then concentrated under reduced pressure. The residue was purified by silical gel chromatography to give the title product (0.7 g, yield: 45.9%); MS (m/z): [M+H]⁺ calcd for C₃₃H₄₆FNO₁₀, 636.31; found, 580.2 (M+H−56). ¹H NMR (400 MHz, Chloroform-d) δ 7.26 (s, 1H), 6.34 (dd, J=10.1, 1.9 Hz, 1H), 6.13 (t, J=1.8 Hz, 1H), 5.10-4.97 (m, 2H), 4.94-4.78 (m, 3H), 4.47-4.37 (m, 1H), 3.49-3.17 (m, 2H), 2.63 (td, J=13.7, 5.9 Hz, 1H), 2.53-2.30 (m, 4H), 2.17-2.07 (m, 2H), 1.86 (dt, J=11.9, 5.4 Hz, 1H), 1.77-1.58 (m, 6H), 1.55 (d, J=1.8 Hz, 3H), 1.49-1.38 (m, 9H), 1.31 (dd, J=7.9, 6.4 Hz, 3H), 1.20 (d, J=4.0 Hz, 3H), 0.96 (d, J=7.1 Hz, 3H).

### Step 3: Preparation of 1-aminopropan-2-yl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride

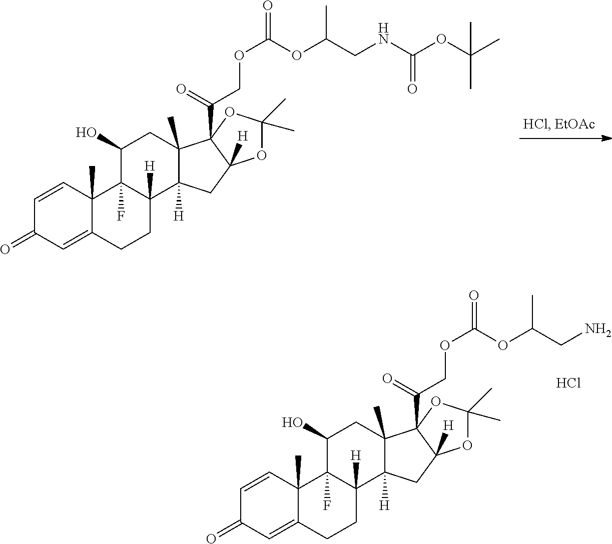

To a stirred solution of tert-butyl (2-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)propyl)carbamate (699 mg, 1.1 mmol) in EtOAc (20 mL) was slowly added 4M HCl in ethyl acetate (4 mL) at ice-bath. The reaction mixture was allowed to stir at room temperature for 18 hours. The solution was diluted with EtOAc and concentrated under reduced pressure to afford the title compound as a white solid (0.62 g, yield: 98.6%) that was used without further purification. MS (m/z): [M+H]⁺ calcd for C₂₈H₃₈FNO₈, 536.26; found, 536.3.

### Step 4: Preparation of Conjugate of HA and 1-aminopropan-2-yl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

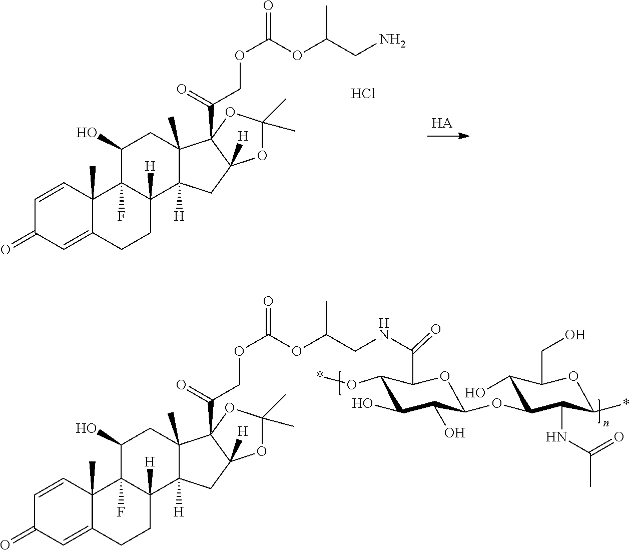

Hyaluronic acid (201 mg, 0.5 mmol carboxylic acid) was dissolved in 22 mL of deionized water in a 100 mL round-bottomed flask followed by the dropwise addition of 20 mL of 1,4-Dioxane while stirring. To the solution was added 1-aminopropan-2-yl (2-((6αS,6βR,7S,8αS,8S,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride (114 mg, 0.2 mmol), N-Hydroxysuccinimide (NHS, 46 mg, 0.4 mmol) and 1-ethyl-3-(3-dimethylaminopropyl)carbodiimide (EDCI, 38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (5 mL) was added to the reaction mixture, after stirring for 1 hour, 1M HCl solution (4.3 mL) was added. NaCl (293 mg, 5 mmol) was then added to the reaction mixture, which was stirred for 1 hour and followed by the dropwise addition of anhydrous alcohol (200 mL) while stirring at −10° C. The mixture was filtered. The filter cake was collected, washed with anhydrous alcohol and dried in vacuo to give the title compound as a white solid. (sodium hyaluronate MW 50 KDa 0.22 g, yield: 71.4%, DSR=17%); ¹H NMR (400 MHz, Deuterium Oxide) δ 7.71-7.61 (m, 0.17H), 6.64-6.50 (m, 0.17H), 6.43-6.31 (m, 0.17H), 5.49-5.33 (m, 0.17H), 5.26-4.98 (m, 0.68H), 4.80-4.44 (m, 2.17H), 4.25-3.09 (m, 10.34H), 2.96-2.50 (m, 0.68H), 2.41-1.73 (m, 3.34H), 1.72-1.53 (m, 1.7H), 1.48-1.34 (m, 1.02H), 1.10-0.91 (m, 0.51H).

With this procedure, reaction of sodium hyaluronate (MW 500 KDa) provided corresponding product (0.19 g, yield: 61.6%, DSR=14%). ¹H NMR (400 MHz, Deuterium Oxide) δ 7.71-7.60 (m, 0.14H), 6.64-6.51 (m, 0.14H), 6.43-6.32 (m, 0.14H), 5.52-5.32 (m, 0.14H), 5.26-4.98 (m, 0.56H), 4.81-4.43 (m, 2.14H), 4.26-3.20 (m, 10.28H), 3.04-2.53 (m, 0.56H), 2.40-1.74 (m, 3.28H), 1.73-1.52 (m, 1.4H), 1.50-1.33 (m, 0.84H), 1.10-0.93 (m, 0.42H).

With this procedure, reaction of sodium hyaluronate (MW 2000 KDa) provided corresponding product (0.16 g, yield: 51.9%, DSR=14%). ¹H NMR (400 MHz, Deuterium Oxide) δ 7.72-7.59 (m, 0.14H), 6.66-6.53 (m, 0.14H), 6.42-6.30 (m, 0.14H), 5.51-5.32 (m, 0.14H), 5.25-4.95 (m, 0.56H), 4.80-4.40 (m, 2.14H), 4.32-3.08 (m, 10.28H), 2.96-2.52 (m, 0.56H), 2.28-1.74 (m, 3.28H), 1.73-1.52 (m, 1.4H), 1.51-1.34 (m, 0.84H), 1.11-0.90 (m, 0.42H).

### Example 21

### Preparation of Conjugate of HA and 2-((2S,6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) piperazine-4-carboxylate

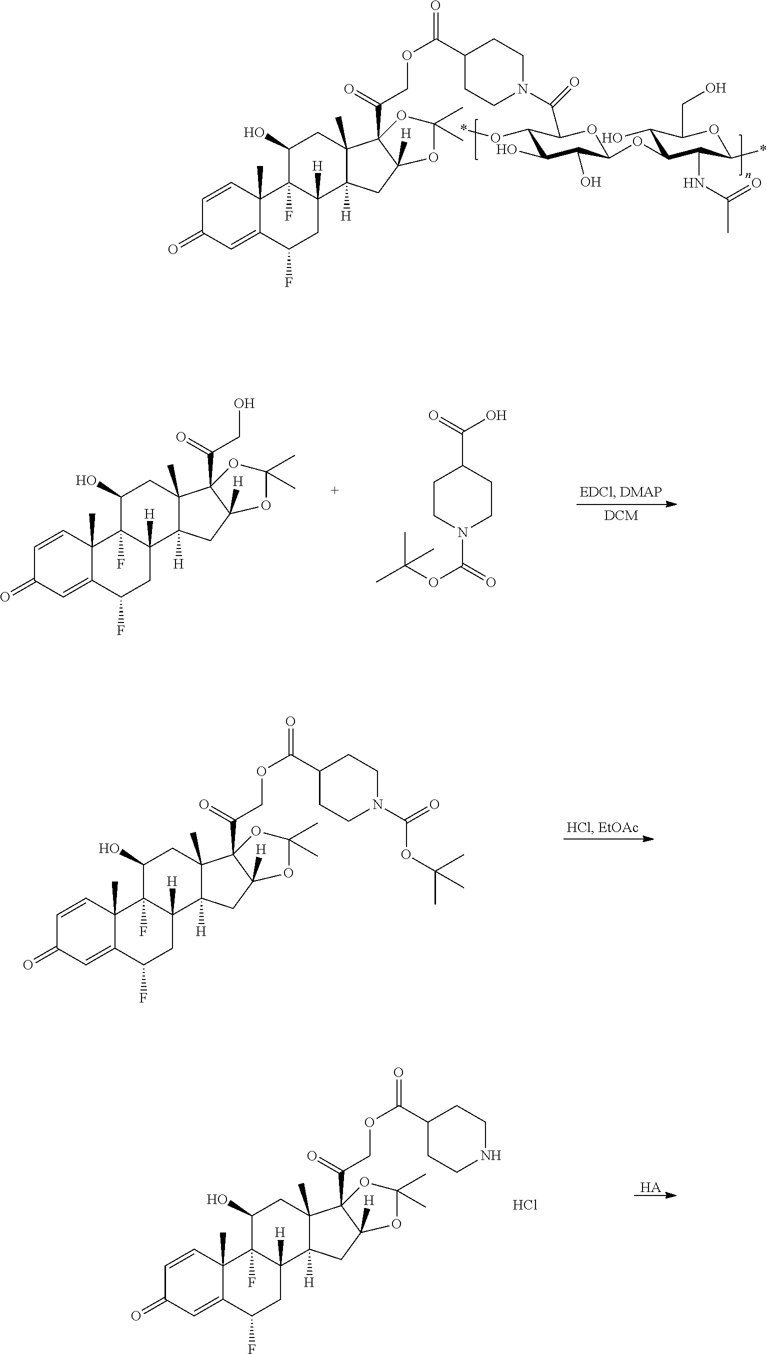

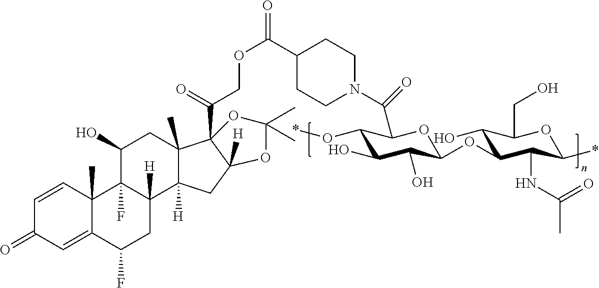

### Step 1: Preparation of 1-(tert-butyl) 4-(2-((2S,6αS,6βR,7S,8αS,8βS,11αR,12αS,12βS)-6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) piperazine-1,4-dicarboxylate

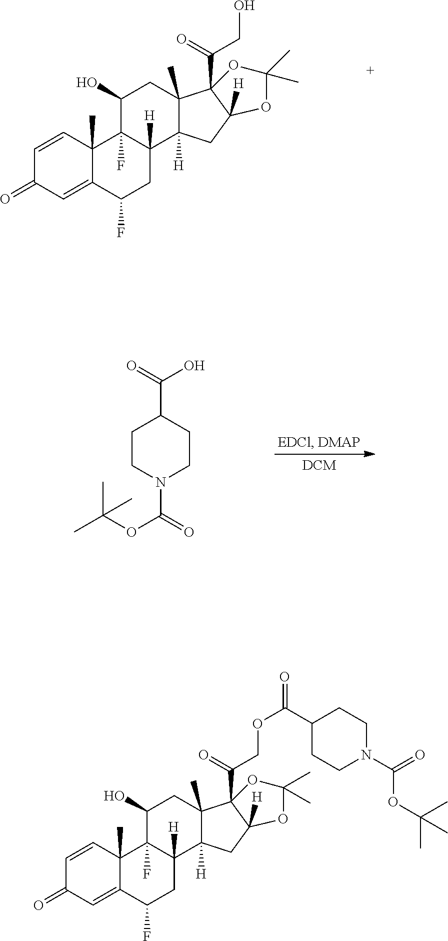

(6α,9α,11β,16β)-6,9-difluoro-11,21-dihydroxy-16,17-[(1-methylethylidene)bis(oxy)]pregna-1,4-diene-3,20-dione (679 mg, 1.5 mmol), 1-(tert-butoxycarbonyl)piperidine-4-carboxylic acid (447 mg, 1.95 mmol), 1-ethyl-3-(3-dimethylaminopropyl)carbodiimide (431 mg, 2.25 mmol), N,N-dimethylpyridin-4-amine (18 mg, 0.15 mmol) was dissolved in DCM (30 mL). The reaction mixture was stirred at room temperature for 3 hours. The solution was diluted with DCM and washed with water and saturated brine solution. The organic layer was dried over sodium sulfate, filtered and then concentrated under reduced pressure. The crude residue was purified by column chromatography (PE/EtOAc=2:1˜1:1) to afford the title compound (940 mg, Yield: 94%). MS (m/z): [M+H]⁺ calcd for C₃₅H₄₇F₂NO₉, 664.32; found, 608.3 (M+H−56). ¹H NMR (400 MHz, Chloroform-d) δ 7.14 (dd, J=10.1, 1.5 Hz, 1H), 6.44 (d, J=2.2 Hz, 1H), 6.38 (dd, J=10.2, 1.9 Hz, 1H), 5.53-5.28 (m, 1H), 5.04-4.84 (m, 3H), 4.42 (dd, J=7.4, 4.3 Hz, 1H), 4.01 (d, J=12.4 Hz, 2H), 2.98-2.85 (m, 2H), 2.66-2.58 (m, 1H), 2.57-2.35 (m, 3H), 2.29 (dd, J=7.1, 5.0 Hz, 1H), 2.19 (td, J=12.5, 6.0 Hz, 1H), 2.02-1.88 (m, 2H), 1.83-1.59 (m, 6H), 1.54 (s, 3H), 1.50-1.40 (m, 12H), 1.23 (s, 3H), 0.93 (s, 3H).

### Step 2: Preparation of 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl piperidine-4-carboxylate hydrochloride

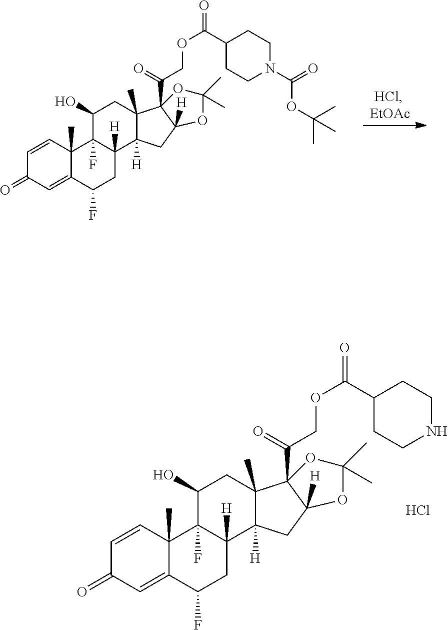

To a stirred solution of 1-(tert-butyl) 4-(2-((2S,6αS,6βR,7S,8αS,8βS,11αR,12αS,12βS)-2,6β-difluoro-7-hydroxy-6α,8α,10,10-tetramethyl-4-oxo-1,2,4,6α,6,7,8,8α,11α,12,12α,12β-dodecahydro-8βH-naphtho[2′,1′:4,5]indeno[1,2-d][1,3]dioxol-8β-yl)-2-oxoethyl) piperidine-1,4-dicarboxylate (800 mg, 1.2 mmol) in EtOAc (32 mL) was slowly added 4M HCl in ethyl acetate (6.4 mL) at ice-bath. The reaction mixture was allowed to cool to room temperature and then was stirred at room temperature for 19 hours. The solution was diluted with EtOAc and concentrated under reduced pressure to afford the title compound as a white solid (0.6 g, yield: 88%) that was used without further purification. MS (m/z): [M+H]⁺ calcd for C₃₀H₃₉F₂NO₇, 564.27; found, 564.3.

### Step 3: Preparation of Conjugate of HA and 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl piperidine-4-carboxylate

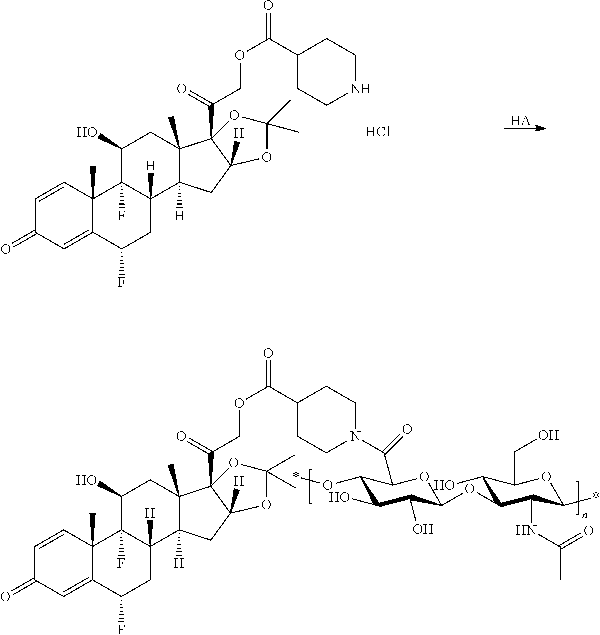

Hyaluronic acid (201 mg, 0.5 mmol carboxylic acid) was dissolved in 22 mL of deionized water in a 100 mL round-bottomed flask followed by the dropwise addition of 22 mL of 1,4-dioxane while stirring. To the solution was added 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl piperidine-4-carboxylate hydrochloride (120 mg, 0.2 mmol), N-Hydroxysuccinimide (46 mg, 0.4 mmol) and 1-ethyl-3-(3-dimethylaminopropyl)carbodiimide (38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (2 mL) was added to the reaction mixture, after stirring for 1 hour, 1M HCl solution (1 mL) was added. NaCl (293 mg, 5 mmol) was then added to the reaction mixture, which was stirred for 1 hour and followed by the dropwise addition of anhydrous alcohol (270 mL) while stirring at −10° C. The mixture was filtered. The filter cake was collected, washed with anhydrous alcohol and dried in vacuo to give the title compound as a white solid. (sodium hyaluronate MW 50 KDa 0.204 g, yield: 65%, DSR=6%); ¹H NMR (400 MHz, Deuterium Oxide) δ 7.55-7.45 (m, 0.06H), 6.55-6.43 (m, 0.12H), 579-5.57 (m, 0.06H), 5.36-5.27 (m, 0.06H), 5.18-4.98 (m, 0.12H), 4.72-4.27 (m, 2.06H), 4.22-3.15 (m, 10.12H), 3.11-2.66 (m, 0.18H), 2.55-2.41 (m, 0.24H), 2.35-1.65 (m, 3.48H), 1.64-1.54 (m, 0.18H), 1.53-1.46 (m, 0.18H), 1.35-1.27 (m, 0.18H), 1.00-0.89 (m, 0.18H).

### Example 22

### Preparation of Conjugate of HA and 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-(aminomethyl)benzoate

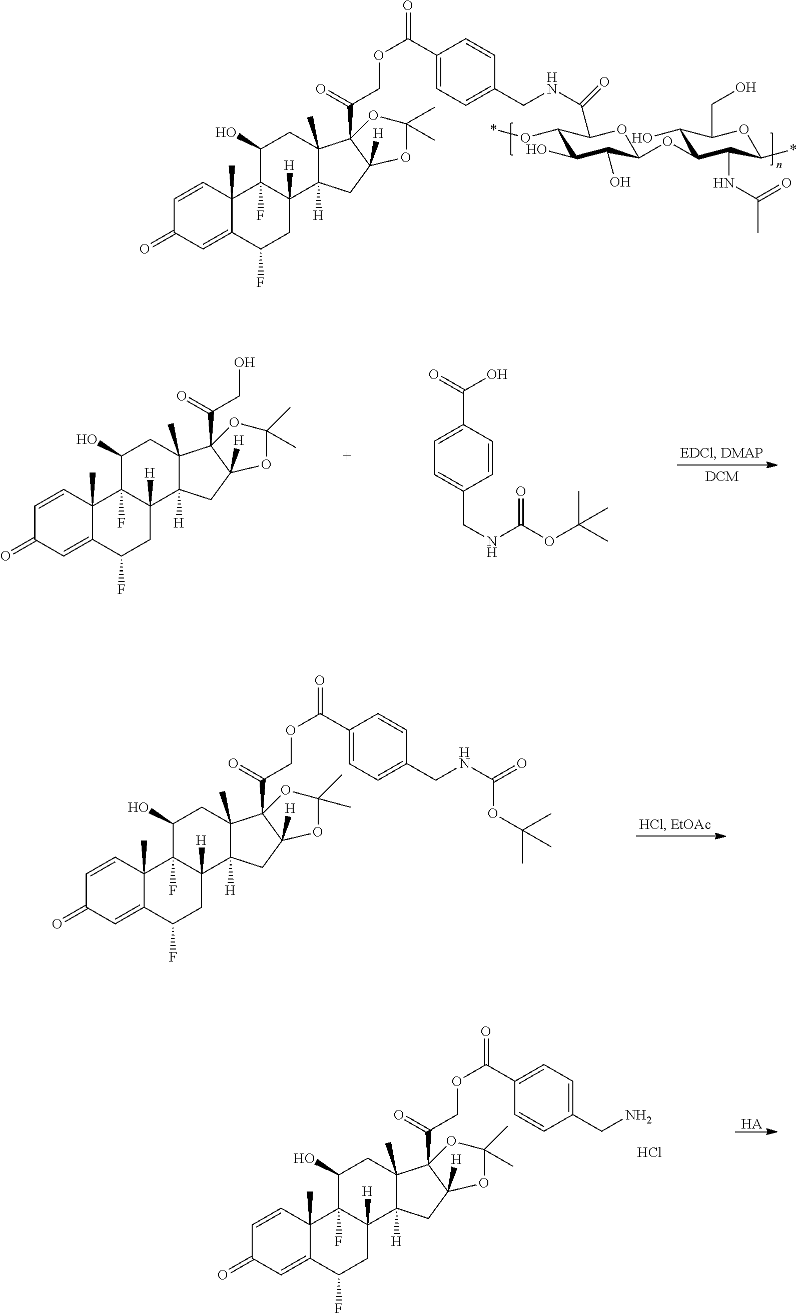

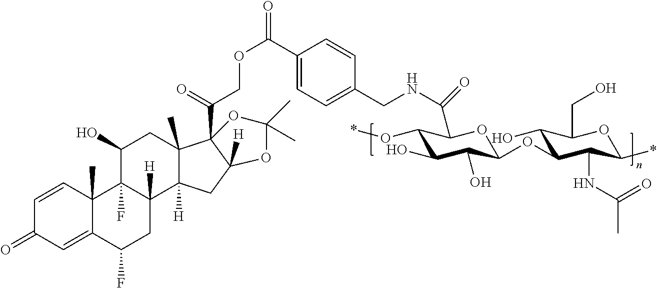

### Step 1: Preparation of 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-(((tert-butoxycarbonyl)amino)methyl)benzoate

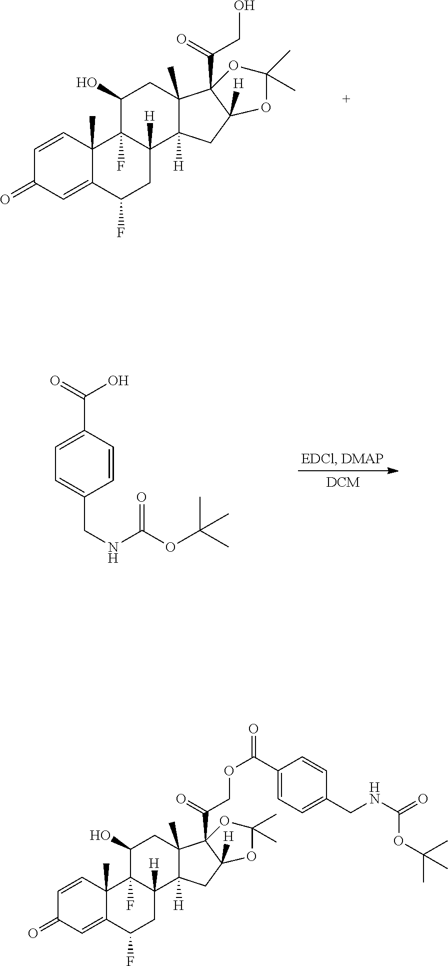

(6α,9α,11β,16β)-6,9-difluoro-11,21-dihydroxy-16,17-[(1-methylethylidene)bis(oxy)]pregna-1,4-diene-3,20-dione (679 mg, 1.5 mmol), 4-(((tert-butoxycarbonyl)amino)methyl)benzoic acid (490 mg, 1.95 mmol), 1-ethyl-3-(3-dimethylaminopropyl)carbodiimide (432 mg, 2.25 mmol), N,N-dimethylpyridin-4-amine (18 mg, 0.15 mmol) was dissolved in DCM (30 mL). The reaction mixture was stirred at room temperature for 3 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=5:1˜2:1) to afford the title compound (1000 mg, Yield: 97%). MS (m/z): [M+H]⁺ calcd for C₃₇H₄₅F₂NO₉, 686.31; found, 630.2 (M+H−56). ¹H NMR (400 MHz, Chloroform-d) δ 8.09-8.00 (m, 2H), 7.37 (d, J=8.0 Hz, 2H), 7.13 (dd, J=10.1, 1.4 Hz, 1H), 6.45 (s, 1H), 6.38 (dd, J=10.1, 1.9 Hz, 1H), 5.45 (ddd, J=11.6, 6.6, 1.9 Hz, 1H), 5.13 (d, J=3.3 Hz, 2H), 5.07-4.92 (m, 2H), 4.50-4.29 (m, 3H), 2.61-2.40 (m, 2H), 2.40-2.16 (m, 3H), 1.90-1.61 (m, 4H), 1.54 (s, 3H), 1.51-1.42 (m, 12H), 1.29 (s, 3H), 1.00 (s, 3H).

### Step 2: Preparation of 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-(aminomethyl)benzoate hydrochloride

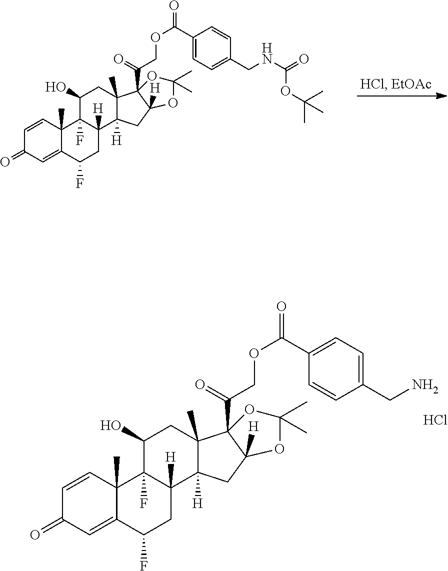

To a stirred solution of 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-(((tert-butoxycarbonyl)amino)methyl)benzoate (800 mg, 1.17 mmol) in EtOAc (32 mL) was slowly added 4M HCl in ethyl acetate (6.4 mL) at ice-bath. The reaction mixture was allowed to cool to room temperature and then was stirred at room temperature for 40 hours. The solution was diluted with EtOAc and concentrated under reduced pressure to afford the title compound as a white solid (0.54 g, yield: 78%) that was used without further purification. MS (m/z): [M+H]⁺ calcd for C₃₂H₃₇F₂NO₇, 586.25; found, 586.4.

### Step 3: Preparation of Conjugate of HA and 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-(aminomethyl)benzoate

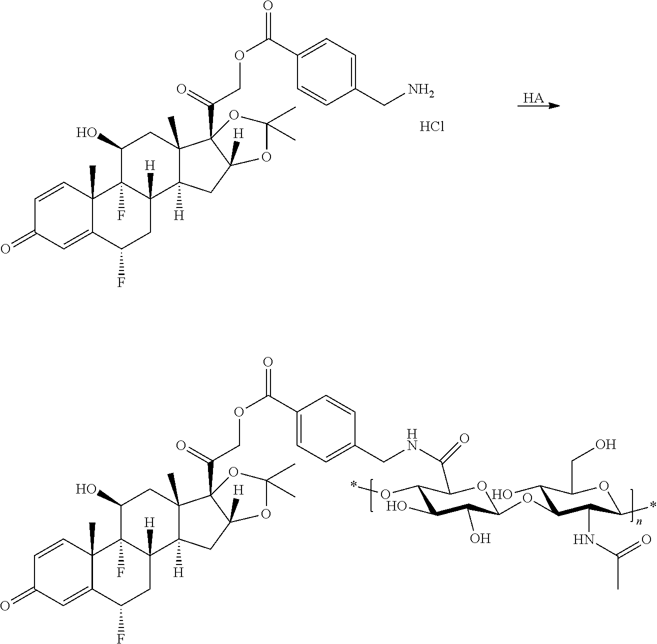

Hyaluronic acid (201 mg, 0.5 mmol carboxylic acid) was dissolved in 22 mL of deionized water in a 100 mL round-bottomed flask followed by the dropwise addition of 22 mL of 1,4-dioxane while stirring. To the solution was added 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-(aminomethyl)benzoate hydrochloride (124 mg, 0.2 mmol), N-hydroxysuccinimide (46 mg, 0.4 mmol) and 1-ethyl-3-(3-dimethylaminopropyl)carbodiimide (38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (2 mL) was added to the reaction mixture, after stirring for 1 hour, 1M HCl solution (1 mL) was added. NaCl (293 mg, 5 mmol) was then added to the reaction mixture, which was stirred for 1 hour and followed by the dropwise addition of anhydrous alcohol (270 mL) while stirring at −10° C. The mixture was filtered. The filter cake was collected, washed with anhydrous alcohol and dried in vacuo to give the title compound as a white solid (sodium hyaluronate MW 50 KDa 0.218 g, yield: 68%, DSR=35%); ¹H NMR (400 MHz, Deuterium Oxide) δ 8.20-8.04 (m, 0.7H), 7.67-7.41 (m, 1.05H), 6.57-6.38 (m, 0.7H), 5.81-5.42 (m, 1.05H), 5.31-5.02 (m, 0.7H), 4.76-4.31 (m, 2.7H), 4.14-3.02 (m, 10H), 2.59-1.66 (m, 5.8H), 1.65-1.43 (m, 2.1H), 1.40-1.27 (m, 1.05H), 1.07-0.87 (m, 1.05H).

### Example 23

### Preparation of Conjugate of HA and 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-aminohexanoate

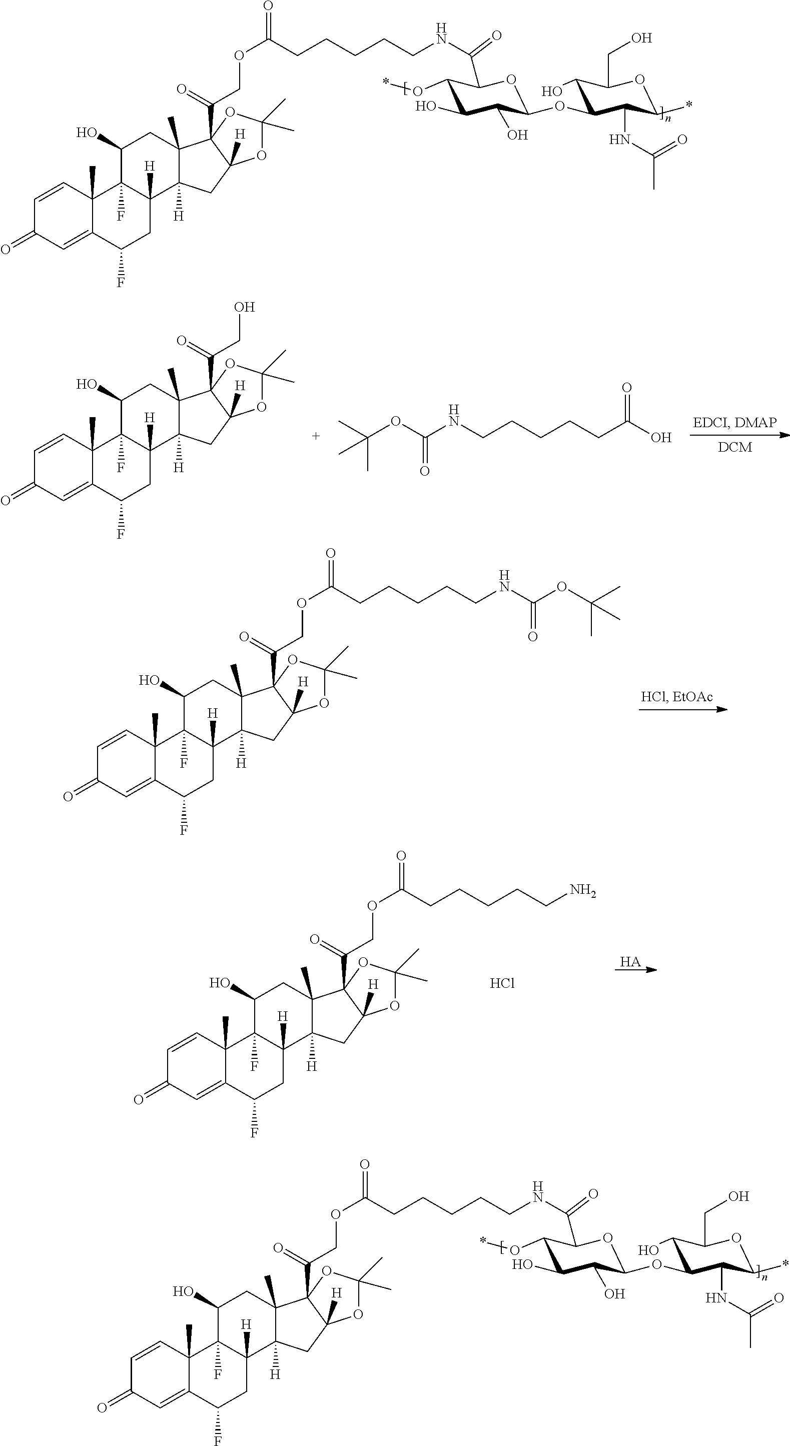

### Step 1: Preparation of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate

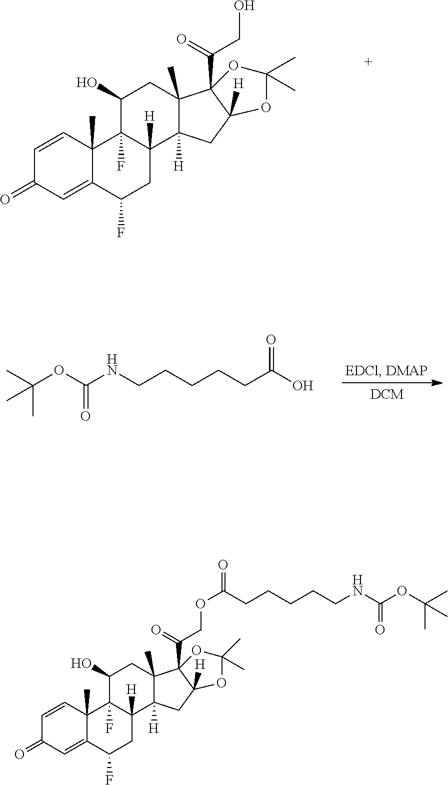

(6α,9α,11β,16β)-6,9-difluoro-11,21-dihydroxy-16,17-[(1-methylethylidene)bis(oxy)]pregna-1,4-diene-3,20-dione (679 mg, 1.5 mmol), 6-((tert-butoxycarbonyl)amino)hexanoic acid (451 mg, 1.95 mmol), 1-ethyl-3-(3-dimethylaminopropyl)carbodiimide (432 mg, 2.25 mmol), N,N-dimethylpyridin-4-amine (18 mg, 0.15 mmol) was dissolved in DCM (20 mL). The reaction mixture was stirred at room temperature for 3 hours. The solution was diluted with DCM and washed with water and saturated brine solution. The organic layer was dried over sodium sulfate, filtered and then concentrated under reduced pressure. The crude residue was purified by column chromatography (PE/Ethyl acetate=2:1˜1:1) to afford the title compound (922 mg, Yield: 92%). MS (m/z): [M+H]⁺ calcd for C₃₅H₄₉F₂NO₉, 666.34; found, 566.3 (M+H−100). ¹H NMR (400 MHz, Chloroform-d) δ 7.15 (dd, J=10.1, 1.4 Hz, 1H), 6.44 (d, J=2.2 Hz, 1H), 6.37 (dd, J=10.2, 1.9 Hz, 1H), 5.33 (ddd, J=11.6, 6.6, 1.9 Hz, 1H), 4.99 (d, J=4.8 Hz, 1H), 4.90 (s, 2H), 4.62 (s, 1H), 4.42 (dt, J=8.5, 2.9 Hz, 1H), 3.12 (q, J=6.8 Hz, 2H), 2.67 (s, 1H), 2.58-2.34 (m, 4H), 2.33-2.25 (m, 1H), 2.24-2.13 (m, 1H), 1.84-1.74 (m, 2H), 1.73-1.59 (m, 4H), 1.57-1.49 (m, 5H), 1.48-1.36 (m, 14H), 1.22 (s, 3H), 0.94 (s, 3H).

### Step 2: Preparation of 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-aminohexanoate hydrochloride

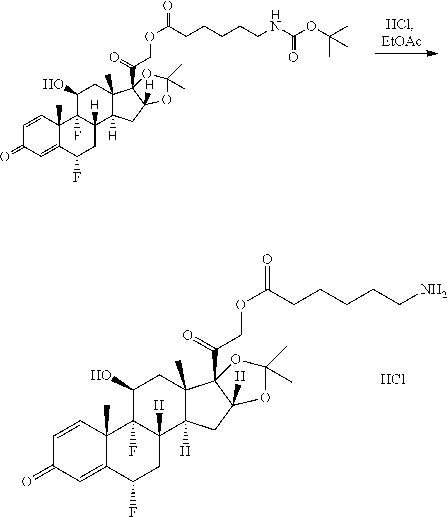

To a stirred solution of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate (800 mg, 1.2 mmol) in EtOAc (32 mL) was slowly added 4M HCl in ethyl acetate (6.4 mL) at ice-bath. The reaction mixture was allowed to cool to room temperature and then was stirred at room temperature for 18 hours. The solution was diluted with EtOAc and concentrated under reduced pressure to afford the title compound as a white solid (0.669 g, yield: 98%) that was used without further purification. MS (m/z): [M+H]⁺ calcd for C₃₀H₄₁F₂NO₇, 566.29; found, 566.3.

### Step 3: Preparation of Conjugate of HA and 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-aminohexanoate

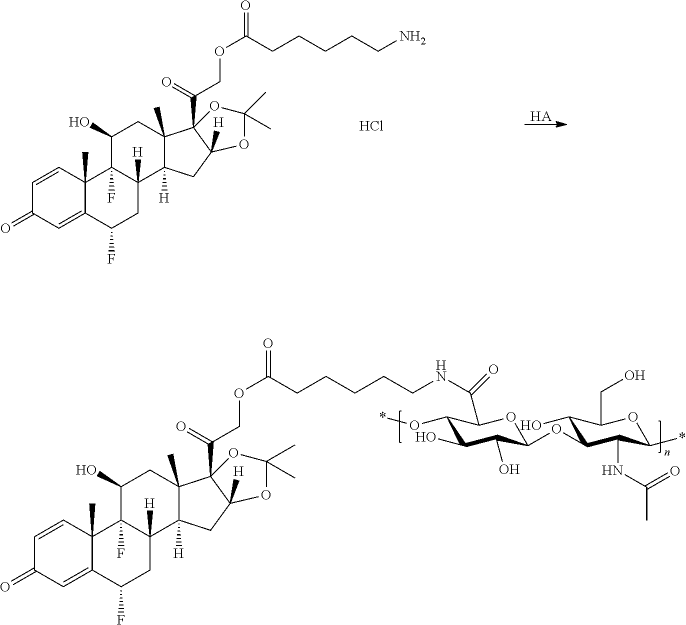

Hyaluronic acid (101 mg, 0.25 mmol carboxylic acid) was dissolved in 11 mL of deionized water in a 100 mL round-bottomed flask followed by the dropwise addition of 11 mL of 1,4-dioxane while stirring. To the solution was added 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-aminohexanoate hydrochloride (150 mg, 0.25 mmol), N-hydroxysuccinimide (58 mg, 0.5 mmol) and 1-ethyl-3-(3-dimethylaminopropyl)carbodiimide (48 mg, 0.25 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (2 mL) was added to the reaction mixture, after stirring for 1 hour, 1M HCl solution (1 mL) was added. NaCl (293 mg, 5 mmol) was then added to the reaction mixture, which was stirred for 1 hour and followed by the dropwise addition of anhydrous alcohol (140 mL) while stirring at −10° C. The mixture was filtered. The filter cake was collected, washed with anhydrous alcohol and dried in vacuo to give the title compound as a white solid. (sodium hyaluronate MW 50 KDa 0.09 g, yield: 37%, DSR=21%); ¹H NMR (400 MHz, Deuterium Oxide) δ 7.56-7.38 (m, 0.21H), 6.55-6.34 (m, 0.42H), 5.78-5.53 (m, 0.21H), 5.33-5.18 (m, 0.42H), 5.13-4.90 (m, 0.42H), 4.70-4.26 (m, 2.21H), 4.19-2.99 (m, 10.42H), 2.60-2.35 (m, 1.05H), 2.28-1.92 (m, 3.21H), 1.90-1.32 (m, 3.15H), 1.31-1.24 (m, 0.63H), 1.00-0.82 (m, 0.63H).

### Example 24

### Preparation of Conjugate of HA and 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 5-aminopentanoate

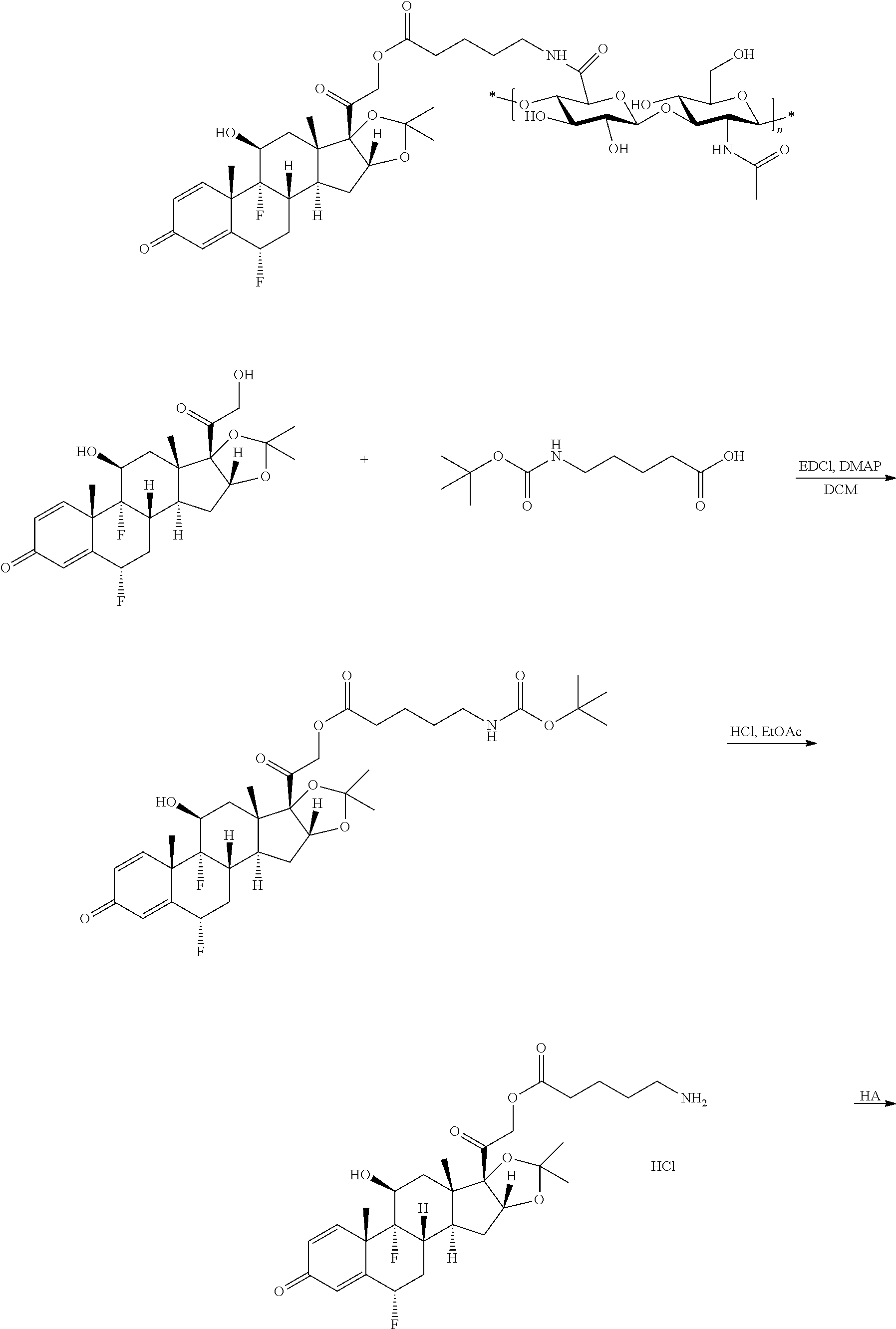

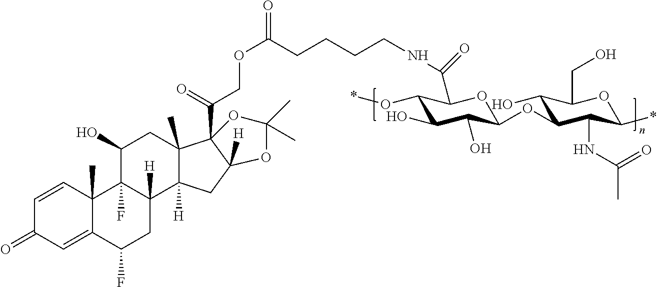

### Step 1: Preparation of 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 5-((tert-butoxycarbonyl)amino)pentanoate

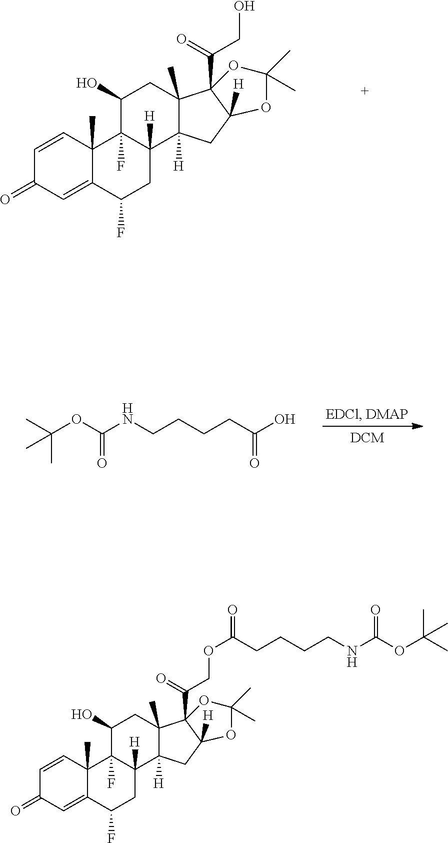

(6α,9α,11β,16β)-6,9-difluoro-11,21-dihydroxy-16,17-[(1-methylethylidene)bis(oxy)]pregna-1,4-diene-3,20-dione (679 mg, 1.5 mmol), 5-((tert-butoxycarbonyl)amino)pentanoic acid (424 mg, 1.95 mmol), 1-ethyl-3-(3-dimethylaminopropyl)carbodiimide (432 mg, 2.25 mmol), N,N-dimethylpyridin-4-amine (18 mg, 0.15 mmol) was dissolved in DCM (20 mL). The reaction mixture was stirred at room temperature for 3 hours. The solution was diluted with DCM and washed with water and saturated brine solution. The organic layer was dried over sodium sulfate, filtered and then concentrated under reduced pressure. The crude residue was purified by column chromatography (PE/EtOAc=2:1˜1:1) to afford the title compound (950 mg, Yield: 97%). MS (m/z): [M+H]⁺ calcd for C₃₄H₄₇F₂NO₉, 652.32; found, 596.3 (M+H−56). ¹H NMR (400 MHz, Chloroform-d) δ 7.15 (dd, J=10.1, 1.5 Hz, 1H), 6.43 (d, J=2.1 Hz, 1H), 6.37 (dd, J=10.2, 1.9 Hz, 1H), 5.50-5.31 (m, 1H), 4.99 (d, J=4.8 Hz, 1H), 4.90 (d, J=6.5 Hz, 2H), 4.65 (s, 1H), 4.42 (dt, J=8.7, 2.8 Hz, 1H), 3.14 (q, J=6.8 Hz, 2H), 2.69 (s, 1H), 2.58-2.40 (m, 3H), 2.40-2.33 (m, 1H), 2.33-2.25 (m, 1H), 2.24-2.13 (m, 1H), 1.86-1.66 (m, 6H), 1.61-1.51 (m, 5H), 1.49-1.39 (m, 12H), 1.22 (s, 3H), 0.94 (s, 3H).

### Step 2: Preparation of 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 5-aminopentanoate hydrochloride

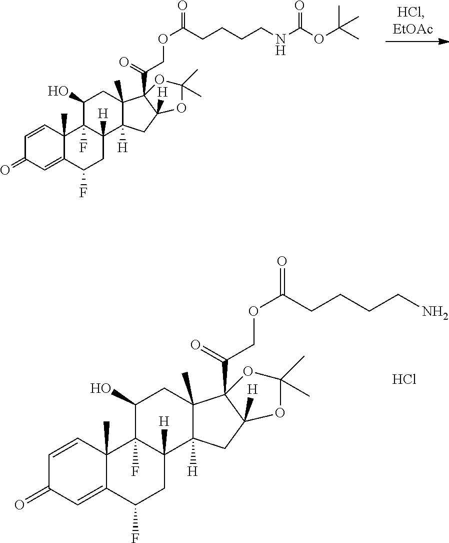

To a stirred solution of 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 5-((tert-butoxycarbonyl)amino)pentanoate (800 mg, 1.23 mmol) in EtOAc (32 mL) was slowly added 4M HCl in ethyl acetate (6.4 mL) at ice-bath. The reaction mixture was allowed to cool to room temperature and then was stirred at room temperature for 18 hours. The solution was diluted with EtOAc and concentrated under reduced pressure to afford the title compound as a white solid (0.67 g, yield: 98%) that was used without further purification. MS (m/z): [M+H]⁺ calcd for C₂₉H₃₉F₂NO₇, 552.27; found, 552.3.

### Step 3: Preparation of Conjugate of HA and 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 5-aminopentanoate

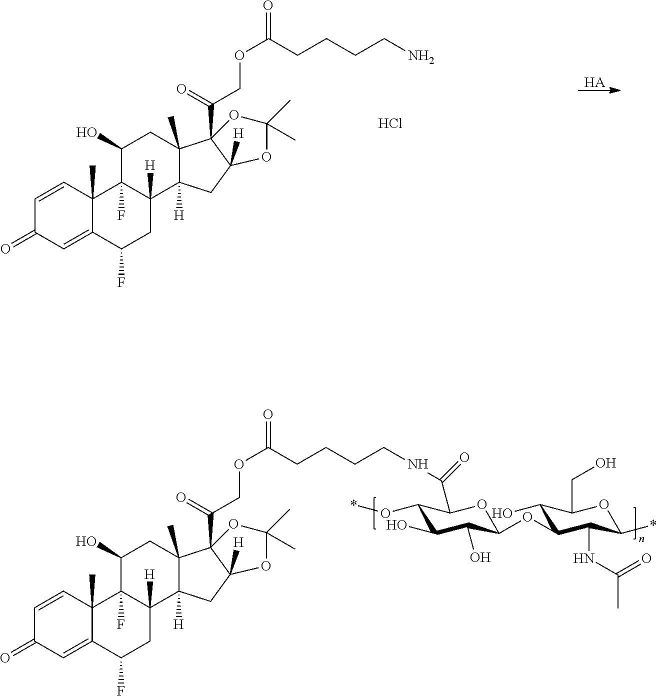

Hyaluronic acid (201 mg, 0.5 mmol carboxylic acid) was dissolved in 22 mL of deionized water in a 100 mL round-bottomed flask followed by the dropwise addition of 22 mL of 1,4-dioxane while stirring. To the solution was added 2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 5-aminopentanoate hydrochloride (118 mg, 0.2 mmol), N-hydroxysuccinimide (46 mg, 0.4 mmol) and 1-ethyl-3-(3-dimethylaminopropyl)carbodiimide (38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (2 mL) was added to the reaction mixture, after stirring for 1 hour, 1M HCl solution (1 mL) was added. NaCl (293 mg, 5 mmol) was then added to the reaction mixture, which was stirred for 1 hour and followed by the dropwise addition of anhydrous alcohol (270 mL) while stirring at −10° C. The mixture was filtered. The filter cake was collected, washed with anhydrous alcohol and dried in vacuo to give the title compound as a white solid. (sodium hyaluronate MW 50 KDa 0.19 g, yield: 61%, DSR=22%); ¹H NMR (400 MHz, Deuterium Oxide) δ 7.59-7.45 (m, 0.22H), 6.59-6.41 (m, 0.44H), 5.82-5.55 (m, 0.22H), 5.36-5.23 (m, 0.22H), 5.16-4.93 (m, 0.66H), 4.73-4.34 (m, 2H), 4.19-3.10 (m, 10.44H), 2.68-2.36 (m, 0.66H), 2.32-1.92 (m, 3.66H), 1.91-1.56 (m, 2.42H), 1.55-1.42 (m, 0.66H), 1.36-1.25 (m, 0.66H), 1.02-0.85 (m, 0.66H).

### Example 25

### Preparation of Conjugate of HA and 5-aminopentyl (2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

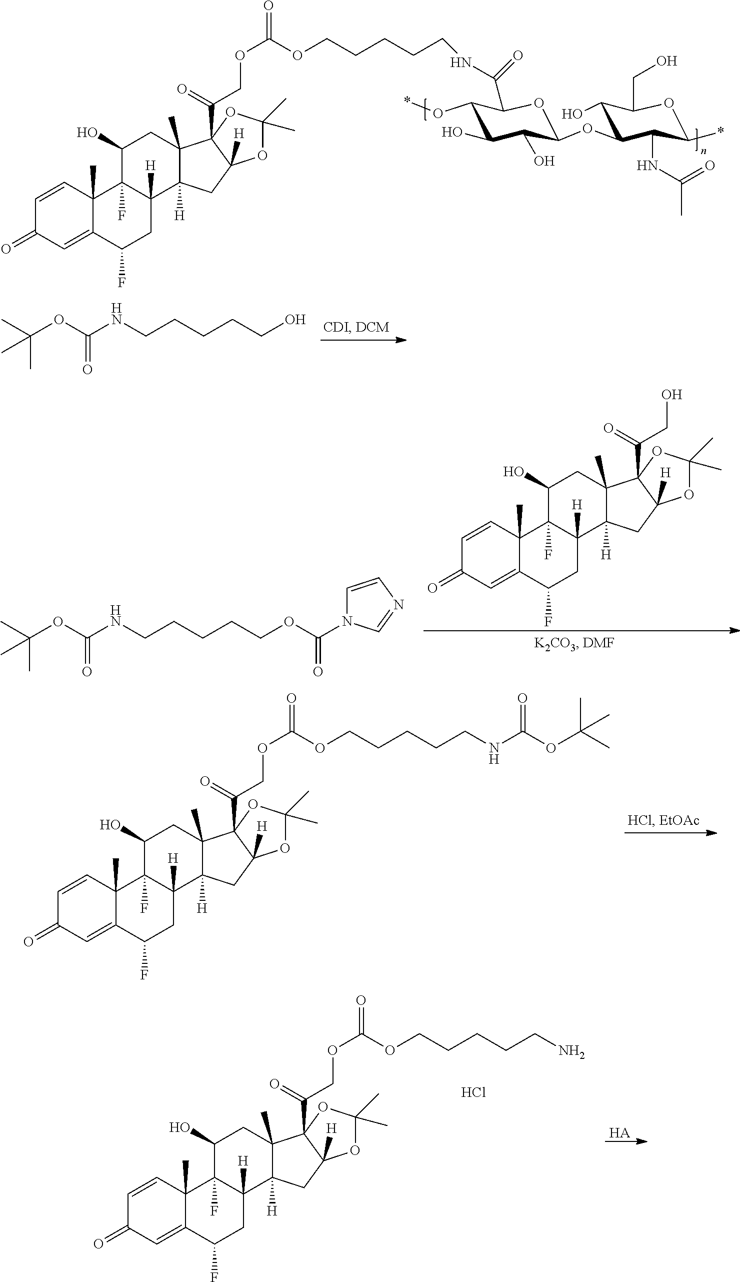

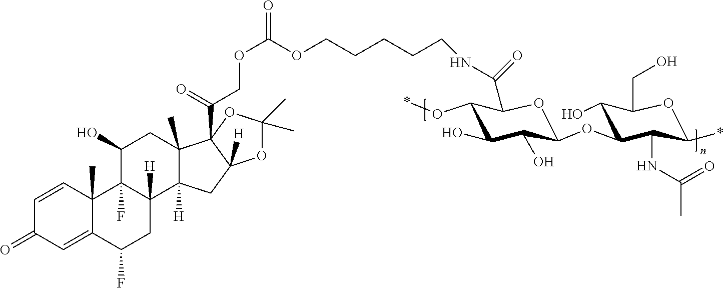

### Step 1: Preparation of 5-((tert-butoxycarbonyl)amino)pentyl 1H-imidazole-1-carboxylate

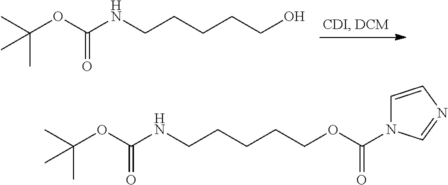 [SMILES: CC(C)(C)OC(=O)NCCCCCO ; CC(C)(C)OC(=O)NCCCCCOC(=O)n1ccnc1]

To a solution of tert-butyl (5-hydroxypentyl)carbamate (407 mg, 2 mmol) in DCM (30 mL) was added 1,1′-carbonyldiimidazole (648 mg, 4 mmol). The resulting mixture was stirred at room temperature for 16 hours. The solution was diluted with DCM and washed with saturated NaHCO₃ solution, water, and saturated brine solution. The organic layer was dried over sodium sulfate, filtered, and then concentrated under reduced pressure to afford the title compound as a yellow oil (0.58 g, yield: 97%) that was used without further purification. MS (m/z): [M+H]⁺ calcd for C₁₋₄H₂₃N₃O₄, 298.17; found, 298.4.

### Step 2: Preparation of tert-butyl (5-(((2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)pentyl)carbamate

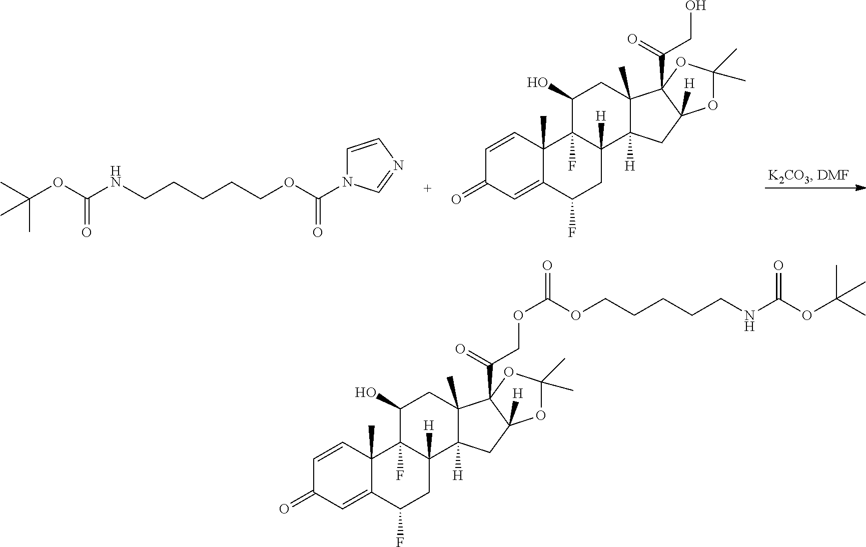

To a solution of (6α,9α,11β,16β)-6,9-difluoro-11,21-dihydroxy-16,17-[(1-methylethylidene)bis(oxy)]pregna-1,4-diene-3,20-dione (905 mg, 2 mmol), in DMF (20 mL) was added 5-((tert-butoxycarbonyl)amino)pentyl 1H-imidazole-1-carboxylate (594 mg, 2 mmol) and K₂CO₃ (276 mg, 2 mmol) under N₂, The resulting mixture was stirred at room temperature for 16 hours. The reaction mixture was poured into water (90 mL) at room temperature, filtered and the filter cake was dissolved in DCM (40 mL). The solution was dried over sodium sulfate, filtered, and then concentrated under reduced pressure. The residue was purified by silical gel chromatography to give the title product (0.8 g, yield: 58%); MS (m/z): [M+H]⁺ calcd for C₃₅H₄₉F₂NO₁₀, 682.33; found, 626.3 (M+H−56). ¹H NMR (400 MHz, Chloroform-d) δ 7.19-7.11 (m, 1H), 6.44 (d, J=2.2 Hz, 1H), 6.37 (dd, J=10.1, 1.9 Hz, 1H), 5.50-5.28 (m, 1H), 5.04-4.94 (m, 2H), 4.84 (d, J=17.9 Hz, 1H), 4.60 (s, 1H), 4.42 (dq, J=8.8, 2.7 Hz, 1H), 4.19 (t, J=6.4 Hz, 2H), 3.12 (q, J=6.7 Hz, 2H), 2.68 (s, 1H), 2.59-2.41 (m, 1H), 2.40-2.25 (m, 2H), 2.23-2.12 (m, 1H), 1.84-1.59 (m, 6H), 1.58-1.49 (m, 5H), 1.48-1.39 (m, 14H), 1.21 (s, 3H), 0.95 (s, 3H).

### Step 3: Preparation of 5-aminopentyl (2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride

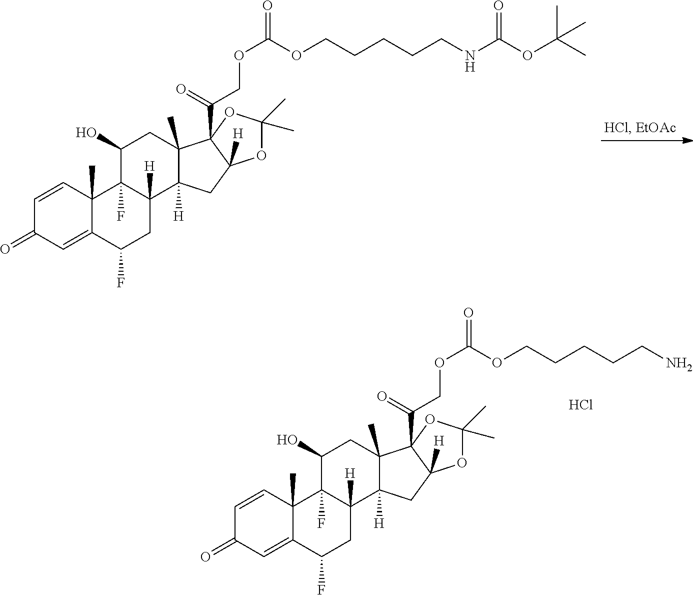

To a stirred solution of tert-butyl (5-(((2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)pentyl)carbamate (600 mg, 0.88 mmol) in EtOAc (24 mL) was slowly added 4M HCl in ethyl acetate (4.8 mL) at ice-bath. The reaction mixture was allowed to cool to room temperature and then was stirred at room temperature for 20 hours. The solution was diluted with EtOAc and concentrated under reduced pressure to afford the title compound as a white solid (0.5 g, yield: 97%) that was used without further purification. MS (m/z): [M+H]⁺ calcd for C₃₀H₄₁F₂NO₈, 582.28; found, 582.3.

### Step 4: Preparation of Conjugate of HA and 5-aminopentyl (2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

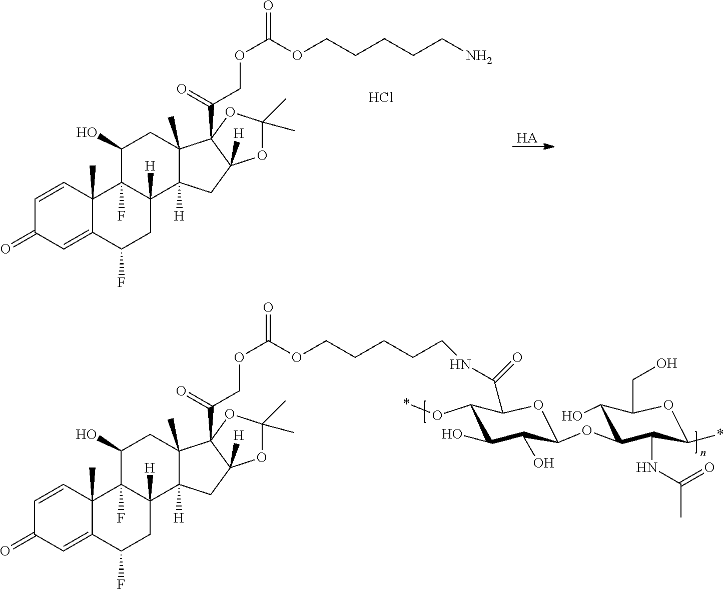

Hyaluronic acid (201 mg, 0.5 mmol carboxylic acid) was dissolved in 22 mL of deionized water in a 100 mL round-bottomed flask followed by the dropwise addition of 22 mL of 1,4-dioxane while stirring. To the solution was added 5-aminopentyl (2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride (124 mg, 0.2 mmol), N-hydroxysuccinimide (46 mg, 0.4 mmol) and 1-ethyl-3-(3-dimethylaminopropyl)carbodiimide (38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (2 mL) was added to the reaction mixture, after stirring for 1 hour, 1M HCl solution (1 mL) was added. NaCl (293 mg, 5 mmol) was then added to the reaction mixture, which was stirred for 1 hour and followed by the dropwise addition of anhydrous alcohol (270 mL) while stirring at −10° C. The mixture was filtered. The filter cake was collected, washed with anhydrous alcohol and dried in vacuo to give the title compound as a white solid. (sodium hyaluronate MW 50 KDa 0.19 g, yield: 59%, DSR=22%); ¹H NMR (400 MHz, Deuterium Oxide) δ 7.58-7.43 (m, 0.22H), 6.59-6.40 (m, 0.44H), 5.82-5.56 (m, 0.22H), 5.39-5.25 (m, 0.22H), 5.17-4.92 (m, 0.44H), 4.72-4.39 (m, 2.66H), 4.37-3.02 (m, 10.44H), 2.61-2.37 (m, 0.66H), 2.30-1.92 (m, 3.22H), 1.91-1.38 (m, 3.52H), 1.37-1.24 (m, 0.66H), 1.02-0.83 (m, 0.66H).

### Example 26

### Preparation of Conjugate of HA and 6-aminohexyl (2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

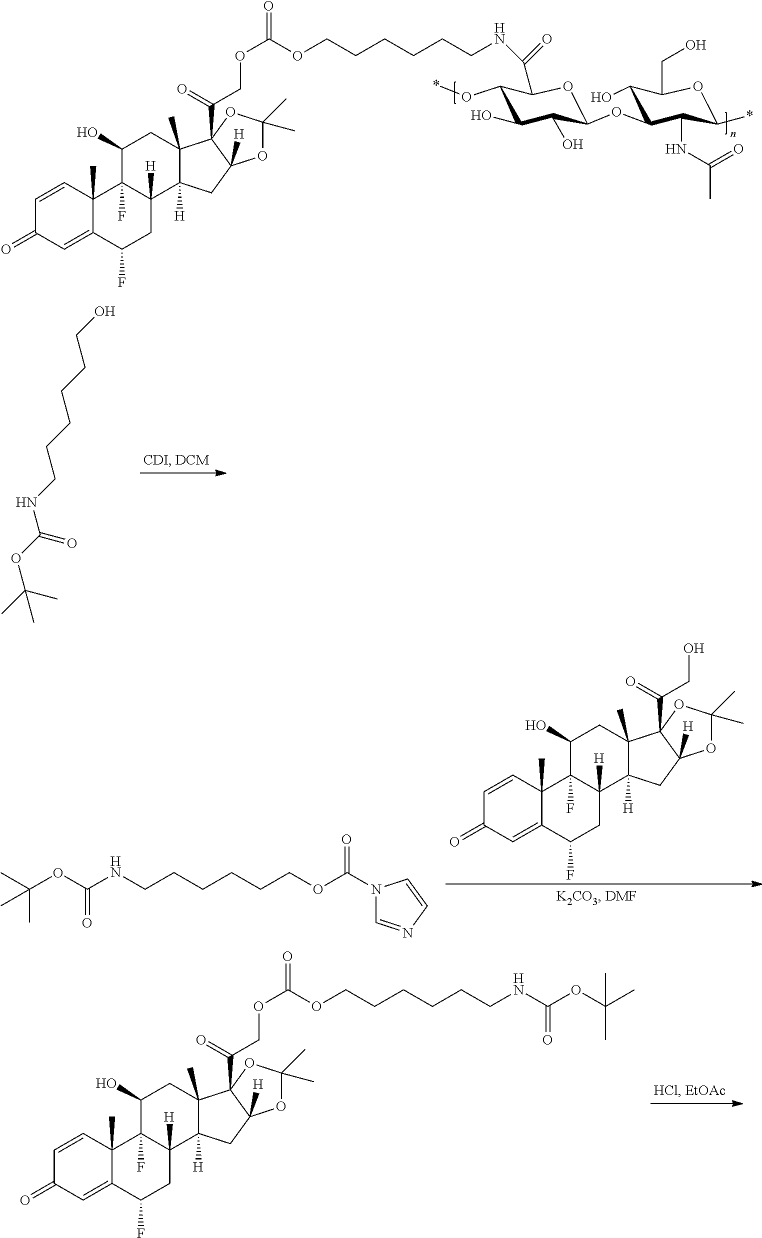

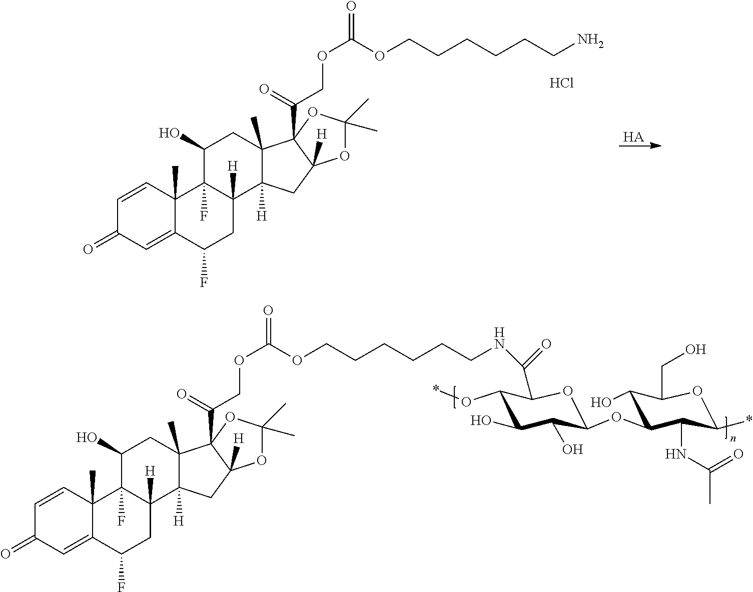

### Step 1: Preparation of 6-((tert-butoxycarbonyl)amino)hexyl 1-imidazole-1-carboxylate

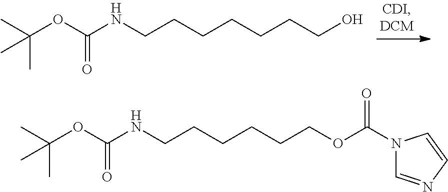 [SMILES: CC(C)(C)OC(=O)NCCCCCCCO ; CC(C)(C)OC(=O)NCCCCCCOC(=O)n1ccnc1]

To a solution of 6-((tert-butoxycarbonyl)amino)hexyl 1H-imidazole-1-carboxylate (435 mg, 2 mmol) in DCM (20 mL) was added 1,1′-carbonyldiimidazole (648 mg, 4 mmol). The resulting mixture was stirred at room temperature for 16 hours. The solution was diluted with DCM and washed with water. The organic layer was dried over sodium sulfate, filtered and then concentrated under reduced pressure to afford the title compound as a yellow oil (0.61 g, yield: 97%) that was used without further purification. MS (m/z): [M+H]⁺ calcd for C₁₅H₂₅N₃O₄, 312.18; found, 312.2

### Step 2: Preparation of tert-butyl (6-(((2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)hexyl)carbamate

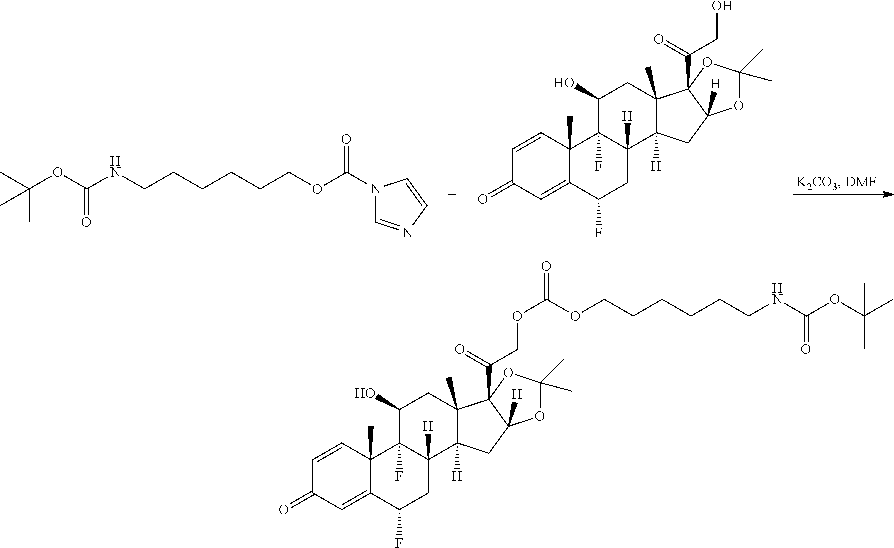

To a solution of (6α,9α,11β,16β)-6,9-difluoro-11,21-dihydroxy-16,17-[(1-methylethylidene)bis(oxy)]pregna-1,4-diene-3,20-dione (311 mg, 1 mmol), in DMF (8 mL) was added 6-((tert-butoxycarbonyl)amino)hexyl 1H-imidazole-1-carboxylate (452 mg, 1 mmol) and K₂CO₃ (138 mg, 1 mmol) under N₂, The resulting mixture was stirred at room temperature for 16 hours. The reaction mixture was poured into water (24 mL) at room temperature, filtered and the filter cake was dissolved in DCM (20 mL). The solution was dried over sodium sulfate, filtered, and then concentrated under reduced pressure. The residue was purified by silical gel chromatography to give the title product (0.45 g, yield: 64%); MS (m/z): [M+H]⁺ calcd for C₃₆H₅₁F₂NO₁₀, 696.35; found, 640.4 (M+H−56). ¹H NMR (400 MHz, Chloroform-d) δ 7.19-7.10 (m, 1H), 6.44 (d, J=2.1 Hz, 1H), 6.37 (dd, J=10.1, 1.9 Hz, 1H), 5.50-5.31 (m, 1H), 5.05-4.94 (m, 2H), 4.83 (d, J=17.8 Hz, 1H), 4.57 (t, J=5.9 Hz, 1H), 4.48-4.36 (m, 1H), 4.19 (t, J=6.4 Hz, 2H), 3.11 (q, J=6.6 Hz, 2H), 2.65-2.42 (m, 2H), 2.37 (d, J=13.9 Hz, 1H), 2.34-2.25 (m, 1H), 2.24-2.13 (m, 1H), 1.88-1.58 (m, 8H), 1.54 (s, 3H), 1.52-1.31 (m, 16H), 1.21 (s, 3H), 0.96 (s, 3H).

### Step 3: Preparation of 6-aminohexyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

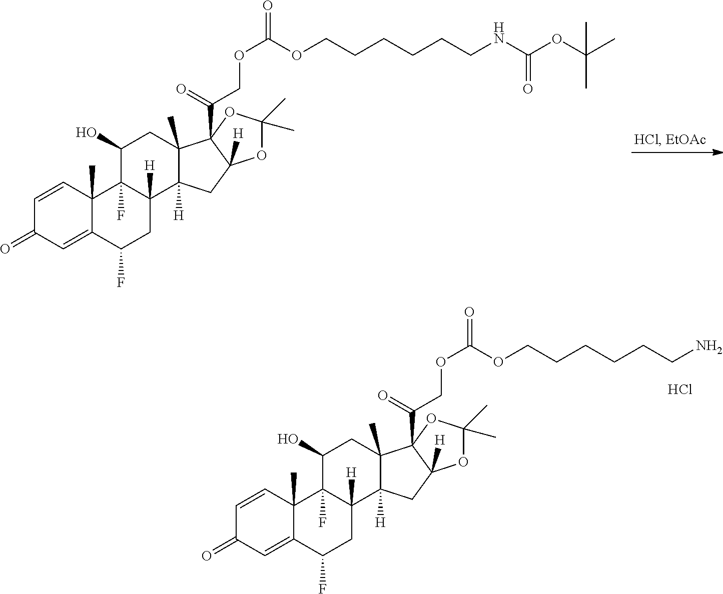

To a stirred solution of tert-butyl (6-(((2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)hexyl)carbamate (430 mg, 0.62 mmol) in EtOAc (17.2 mL) was slowly added 4M HCl in ethyl acetate (3.4 mL) at ice-bath. The reaction mixture was allowed to cool to room temperature and then was stirred at room temperature for 21 hours. The solution was diluted with EtOAc and concentrated under reduced pressure to afford the title compound as a white solid (0.39 g, yield: 99%) that was used without further purification. MS (m/z): [M+H]⁺ calcd for C₃₁H₄₃F₂NO₈, 596.30; found, 596.3.

### Step 4: Preparation of Conjugate of HA and 6-aminohexyl (2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

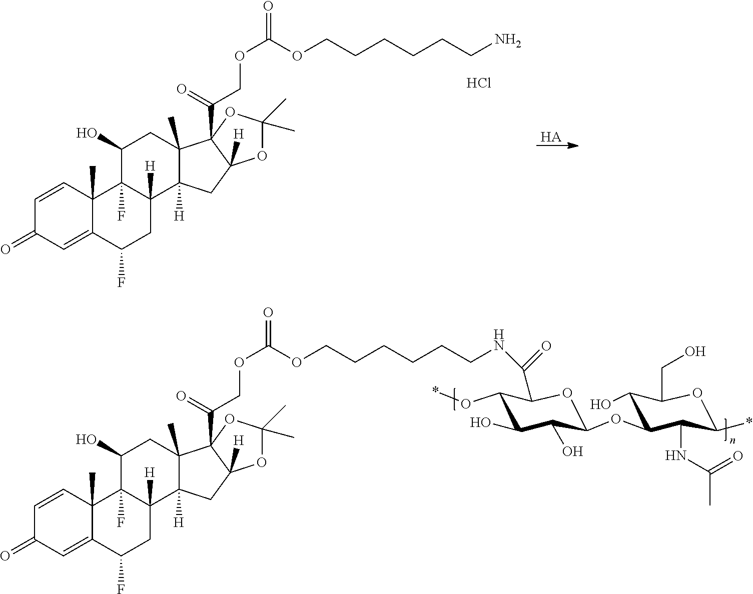

Hyaluronic acid (201 mg, 0.5 mmol carboxylic acid) was dissolved in 22 mL of deionized water in a 100 mL round-bottomed flask followed by the dropwise addition of 22 mL of 1,4-dioxane while stirring. To the solution was added 6-aminohexyl (2-((2S,6αS,6βR,7S,8αS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride (126 mg, 0.2 mmol), N-hydroxysuccinimide (46 mg, 0.4 mmol) and 1-ethyl-3-(3-dimethylaminopropyl)carbodiimide (38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (2 mL) was added to the reaction mixture, after stirring for 1 hour, 1M HCl solution (1 mL) was added. NaCl (293 mg, 5 mmol) was then added to the reaction mixture, which was stirred for 1 hour and followed by the dropwise addition of anhydrous alcohol (300 mL) while stirring at −10° C. The mixture was filtered. The filter cake was collected, washed with anhydrous alcohol and dried in vacuo to give the title compound as a white solid. (sodium hyaluronate MW 500 KDa 0.19 g, yield: 59%, DSR=15%); ¹H NMR (400 MHz, Deuterium Oxide) δ 7.59-7.47 (m, 0.15H), 6.59-6.43 (m, 0.3H), 5.79-5.58 (m, 0.15H), 5.39-5.25 (m, 0.15H), 5.16-4.91 (m, 0.3H), 4.71-4.37 (m, 2.45H), 4.36-2.88 (m, 10.3H), 2.59-2.39 (m, 0.45H), 2.32-1.66 (m, 3.15H), 1.65-1.34 (m, 2.7H), 1.33-1.26 (m, 0.45H), 1.02-0.86 (m, 0.45H).

### Example 27

### Preparation of Conjugate of HA and 6-aminohexyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

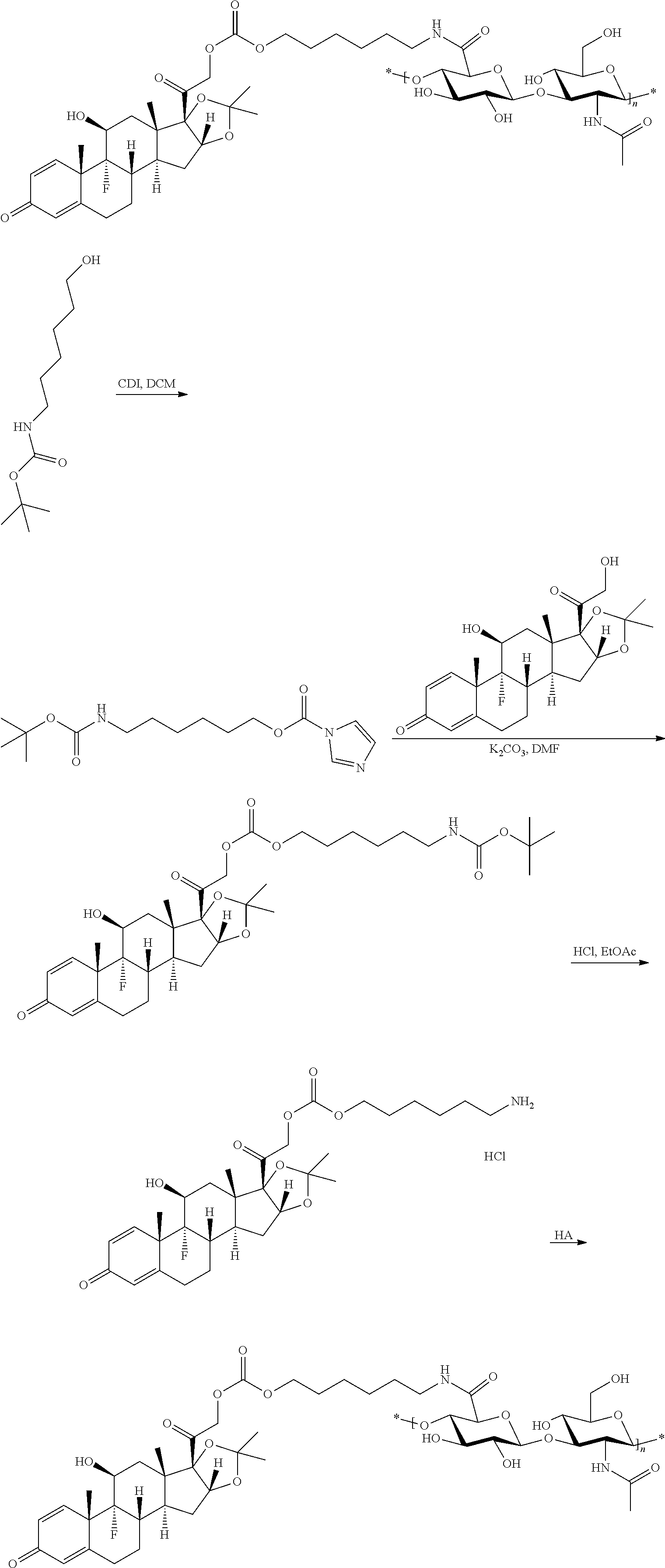

### Step 1: Preparation of 6-((tert-butoxycarbonyl)amino)hexyl 1H-imidazole-1-carboxylate

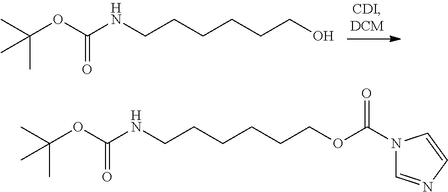 [SMILES: CC(C)(C)OC(=O)NCCCCCCO ; CC(C)(C)OC(=O)NCCCCCCOC(=O)n1ccnc1]

To a solution of 6-((tert-butoxycarbonyl)amino)hexyl 1H-imidazole-1-carboxylate (435 mg, 2 mmol) in DCM (20 mL) was added 1,1′-carbonyldiimidazole (648 mg, 4 mmol). The resulting mixture was stirred at room temperature for 16 hours. The solution was diluted with DCM and washed with water. The organic layer was dried over sodium sulfate, filtered and then concentrated under reduced pressure to afford the title compound as a yellow oil (0.61 g, yield: 97%) that was used without further purification. MS (m/z): [M+H]⁺ calcd for C₁₅H₂₅N₃O₄, 312.18; found, 312.2.

### Step 2: Preparation of tert-butyl (6-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)hexyl)carbamate

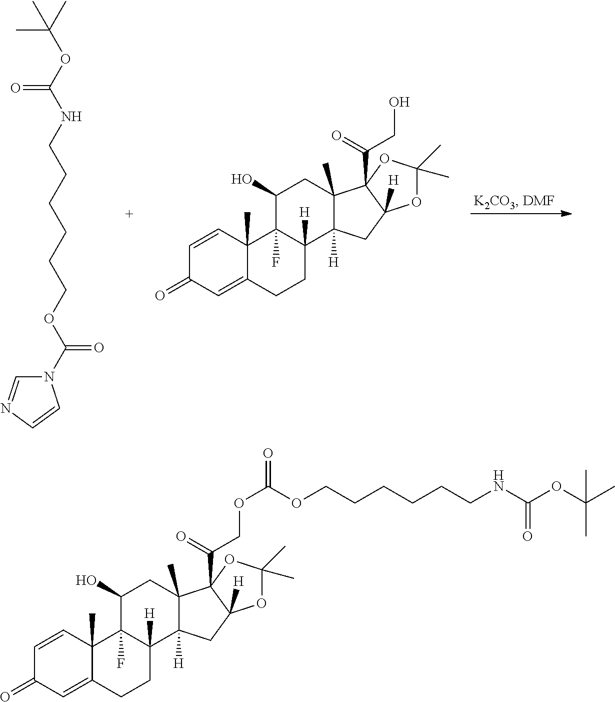

To a solution of 9α-fluoro-11β,16α,17,21-tetrahydroxy-1,4-pregnadiene-3,20-dione (435 mg, 1 mmol) in DMF (8 mL) was added 6-((tert-butoxycarbonyl)amino)hexyl 1H-imidazole-1-carboxylate (311 mg, 1 mmol) and K₂CO₃ (414 mg, 13 mmol) under N₂, the resulting mixture was stirred at room temperature for 16 hours. The reaction mixture was poured into water (25 mL) at room temperature, filtered and the filter cake was dissolved in DCM (20 mL). The solution was dried over sodium sulfate, filtered, and then concentrated under reduced pressure. The residue was purified by silical gel chromatography to give the title product (0.35 g, yield: 51%); MS (m/z): [M+H]⁺ calcd for C₃₆H₅₂FNO₁₀, 678.36; found, 622.4 (M+H−56). ¹H NMR (400 MHz, Chloroform-d) δ 7.21 (d, J=10.1 Hz, 1H), 6.34 (dd, J=10.2, 1.9 Hz, 1H), 6.14 (t, J=1.7 Hz, 1H), 5.05-4.95 (m, 2H), 4.83 (d, J=17.8 Hz, 1H), 4.55 (s, 1H), 4.42 (d, J=8.8 Hz, 1H), 4.19 (t, J=6.5 Hz, 2H), 3.11 (q, J=6.7 Hz, 2H), 2.72-2.59 (m, 1H), 2.55-2.34 (m, 3H), 2.21 (s, 1H), 2.16-2.06 (m, 5.9 Hz, 1H), 1.92-1.82 (m, 1H), 1.75-1.59 (m, 8H), 1.55 (s, 3H), 1.52-1.32 (m, 16H), 1.21 (s, 3H), 0.96 (s, 3H).

### Step 3: Preparation of 6-aminohexyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

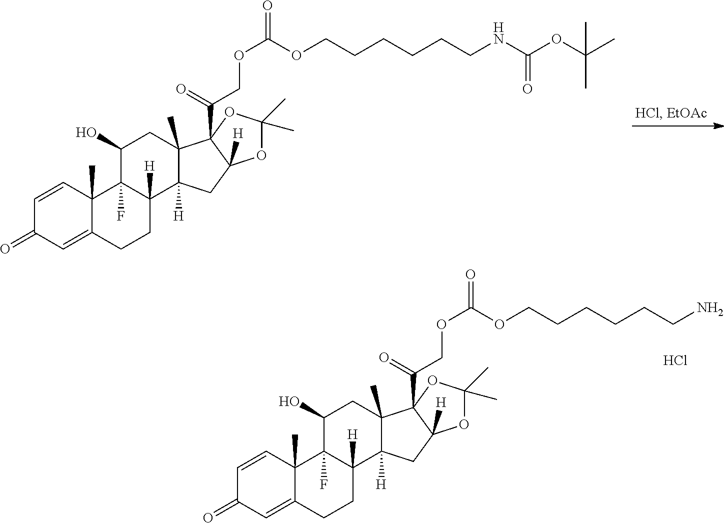

To a stirred solution of tert-butyl (6-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)hexyl)carbamate (330 mg, 0.48 mmol) in EtOAc (13.2 mL) was slowly added 4M HCl in ethyl acetate (2.6 mL) at ice-bath. The reaction mixture was allowed to cool to room temperature and then was stirred at room temperature for 21 hours. The solution was diluted with EtOAc and concentrated under reduced pressure to afford the title compound as a white solid (0.28 g, yield: 94%) that was used without further purification. MS (m/z): [M+H]⁺ calcd for C₃₁H₄₄FNO₈, 578.31; found, 578.3.

### Step 4: Preparation of Conjugate of HA and 6-aminohexyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

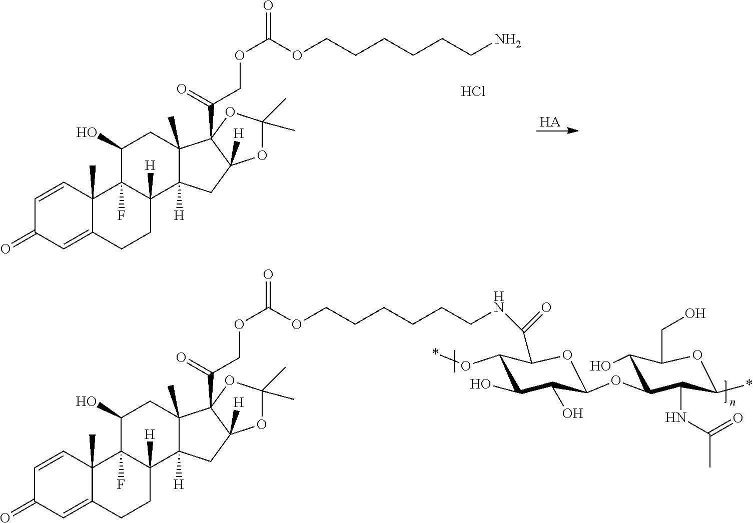

Hyaluronic acid (201 mg, 0.5 mmol carboxylic acid) was dissolved in 22 mL of deionized water in a 100 mL round-bottomed flask followed by the dropwise addition of 22 mL of 1,4-dioxane while stirring. To the solution was added 6-aminohexyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride (123 mg, 0.2 mmol), N-hydroxysuccinimide (46 mg, 0.4 mmol) and 1-ethyl-3-(3-dimethylaminopropyl)carbodiimide (38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (2 mL) was added to the reaction mixture, after stirring for 1 hour, 1M HCl solution (1.7 mL) was added. NaCl (293 mg, 5 mmol) was then added to the reaction mixture, which was stirred for 1 hour and followed by the dropwise addition of anhydrous alcohol (300 mL) while stirring at −10° C. The mixture was filtered. The filter cake was collected, washed with anhydrous alcohol and dried in vacuo to give the title compound as a white solid. (sodium hyaluronate MW 500 KDa 0.21 g, yield: 66%, DSR=15%); ¹H NMR (400 MHz, Deuterium Oxide) δ 7.62-7.51 (m, 0.15H), 6.53-6.42 (m, 0.15H), 6.33-6.22 (m, 0.15H), 5.38-5.24 (m, 0.15H), 5.15-4.92 (m, 0.3H), 4.74-4.21 (m, 2.45H), 4.20-3.00 (m, 10.3H), 2.87-2.45 (m, 0.6H), 2.34-1.66 (m, 3H), 1.65-1.35 (m, 3H), 1.34-1.27 (m, 0.45H), 1.02-0.86 (m, 0.45H).

### Example 28

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2-(2-aminoethoxy)acetate

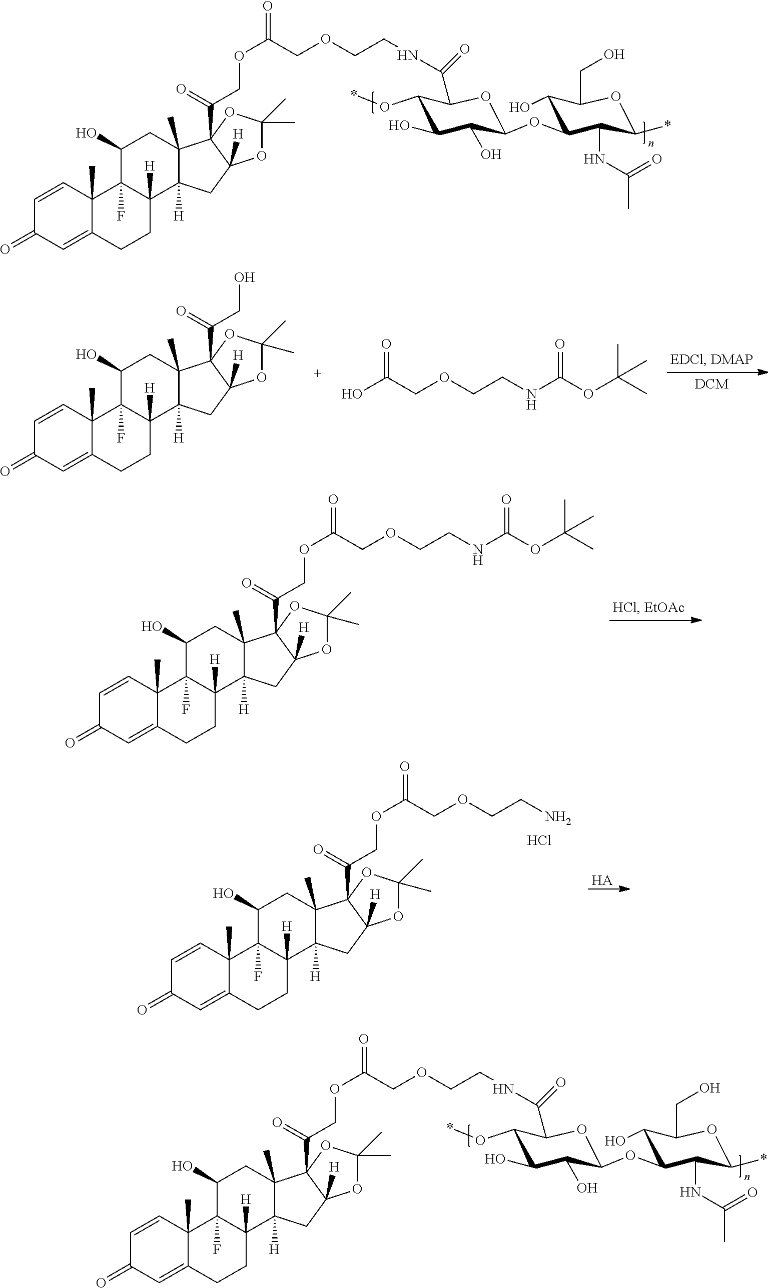

### Step 1: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2-(2-((tert-butoxycarbonyl)amino)ethoxy)acetate

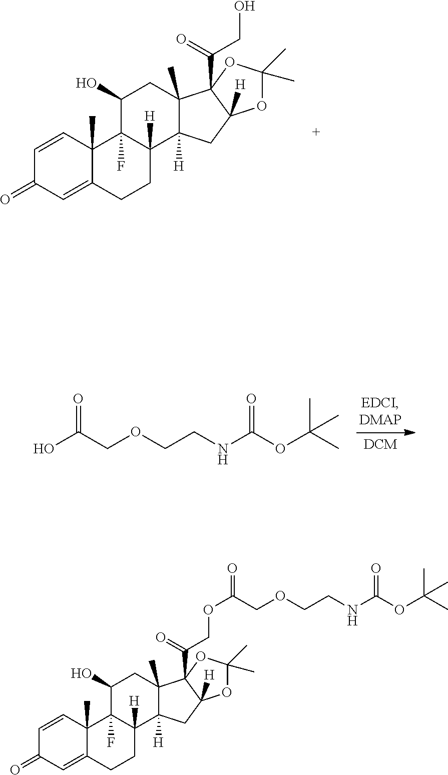

To a mixture of 9α-fluoro-11β,16α,17,21-tetrahydroxy-1,4-pregnadiene-3,20-dione (652 mg, 1.5 mmol), 2-(2-((tert-butoxycarbonyl)amino)ethoxy)acetic acid (428 mg, 1.95 mmol), 1-ethyl-3-(3-dimethylaminopropyl)carbodiimide (432 mg, 2.25 mmol), N,N-dimethylpyridin-4-amine (18 mg, 0.15 mmol) was dissolved in DCM (20 mL). The reaction mixture was stirred at room temperature for 3 hours. The solution was diluted with DCM and washed with water and saturated brine solution. The organic layer was dried over sodium sulfate, filtered, and then concentrated under reduced pressure. The crude residue was purified by column chromatography (PE/EtOAc=2:1˜1:1) to afford the title compound (900 mg, Yield: 95%). MS (m/z): [M+H]⁺ calcd for C₃₃H₄₆FNO₁₀, 636.31; found, 580.3 (M+H−56). ¹H NMR (400 MHz, Chloroform-d) δ 7.22 (d, J=10.3 Hz, 1H), 6.35 (dd, J=10.1, 1.9 Hz, 1H), 6.13 (t, J=1.8 Hz, 1H), 5.17-4.87 (m, 3H), 4.53-4.37 (m, 1H), 4.26 (d, J=4.3 Hz, 2H), 3.65 (t, J=5.0 Hz, 2H), 3.36 (s, 2H), 2.72-2.57 (m, 1H), 2.54-2.26 (m, 3H), 2.17-2.06 (m, 1H), 1.96-1.81 (m, 1H), 1.79-1.59 (m, 6H), 1.56 (s, 3H), 1.49-1.40 (m, 12H), 1.22 (s, 3H), 0.95 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2-(2-aminoethoxy)acetate

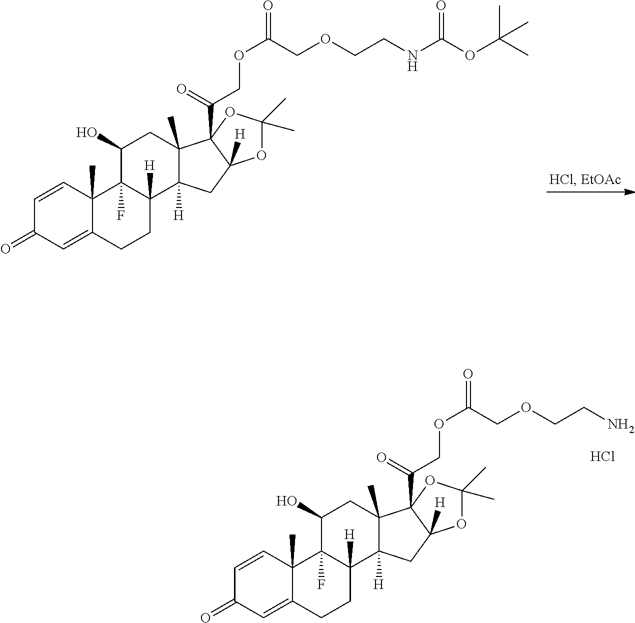

To a stirred solution of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2-(2-((tert-butoxycarbonyl)amino)ethoxy)acetate (600 mg, 0.94 mmol) in EtOAc (24 mL) was slowly added 4M HCl in ethyl acetate (4.8 mL) at ice-bath. The reaction mixture was allowed to cool to room temperature and then was stirred at room temperature for 21 hours. The solution was diluted with EtOAc and concentrated under reduced pressure to afford the title compound as a white solid (0.46 g, yield: 91%) that was used without further purification. MS (m/z): [M+H]⁺ calcd for C₂₈H₃₈FNO₈, 536.26; found, 536.1.

### Step 3: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2-(2-aminoethoxy)acetate

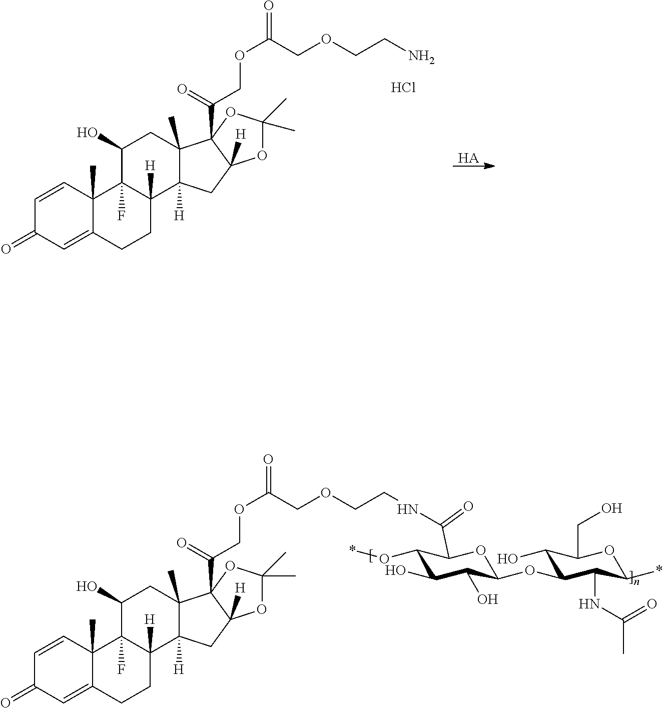

Hyaluronic acid (201 mg, 0.5 mmol carboxylic acid) was dissolved in 22 mL of deionized water in a 100 mL round-bottomed flask followed by the dropwise addition of 22 mL of 1,4-dioxane while stirring. To the solution was added 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2-(2-aminoethoxy)acetate hydrochloride (114 mg, 0.2 mmol), N-hydroxysuccinimide (46 mg, 0.4 mmol) and 1-ethyl-3-(3-dimethylaminopropyl)carbodiimide (38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (2 mL) was added to the reaction mixture, after stirring for 1 hour, 1M HCl solution (1.3 mL) was added. NaCl (293 mg, 5 mmol) was then added to the reaction mixture, which was stirred for 1 hour and followed by the dropwise addition of anhydrous alcohol (300 mL) while stirring at −10° C. The mixture was filtered. The filter cake was collected, washed with anhydrous alcohol, and dried in vacuo to give the title compound as a white solid. (sodium hyaluronate MW 50 KDa 0.199 g, yield: 64%, DSR=15%); ¹H NMR (400 MHz, Deuterium Oxide) δ 7.65-7.55 (m, 0.15H), 6.55-6.44 (m, 0.15H), 6.34-6.21 (m, 0.15H), 5.45-5.32 (m, 0.15H), 5.17-5.01 (m, 0.3H), 4.71-4.37 (m, 2.45H), 4.23-3.03 (m, 10.6H), 2.89-2.45 (m, 0.6H), 2.32-1.69 (m, 3.9H), 1.66-1.46 (m, 0.9H), 1.38-1.29 (m, 0.45H), 1.03-0.89 (m, 0.45H).

### Example 29

### Preparation of Conjugate of HA and 4-(2-aminoethyl)phenyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

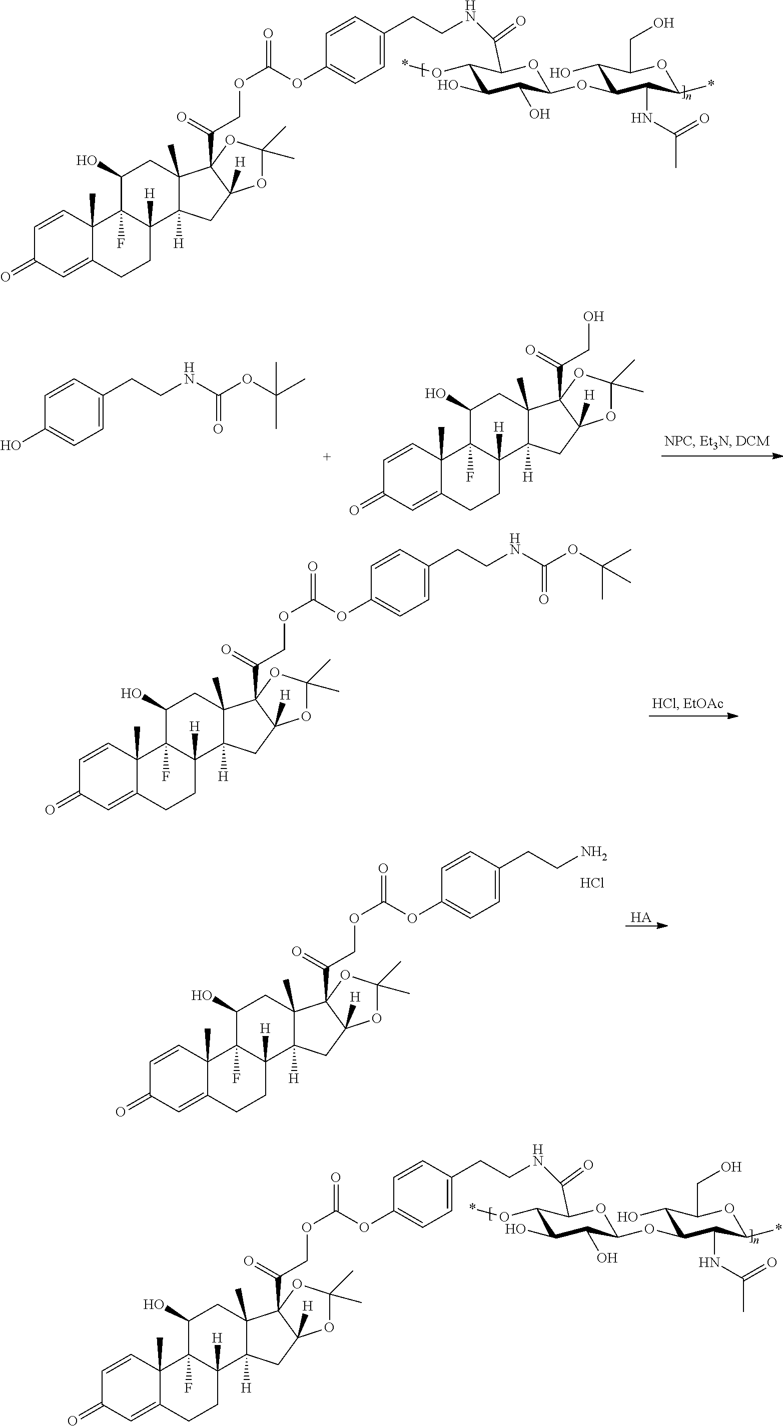

### Step 1: Preparation of tert-butyl (4-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12b-dodecahydro-8βH-naphtho[2′,1′:4,5]indeno[1,2-d][1,3]dioxol-8β-yl)-2-oxoethoxy)carbonyl)oxy)butyl)carbamate

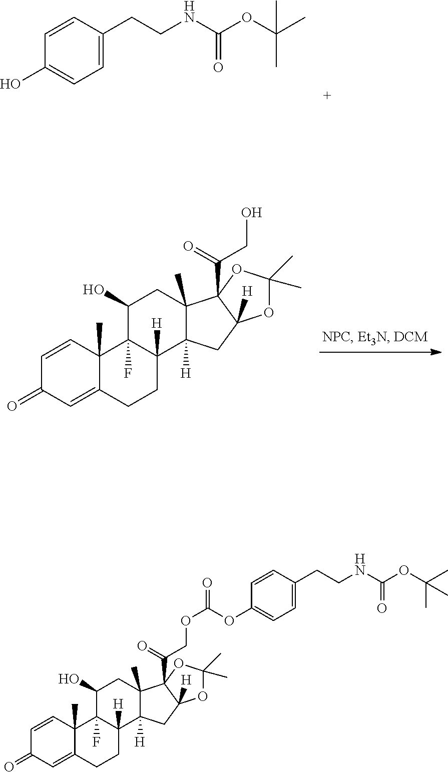

To a mixture of tert-butyl (4-hydroxyphenethyl)carbamate (711 mg, 3 mmol), and bis (4-nitrophenyl) carbonate (1094 mg, 3.6 mmol) in dichloromethane (30 mL) was added triethylamine (758 mg, 7.5 mmol) under N₂, the reaction mixture was heated to reflux for 7 hours. Then 9α-fluoro-11β,16α,17,21-tetrahydroxy-1,4-pregnadiene-3,20-dione (1042 mg, 2.4 mmol) was added and the resulting mixture was stirred at room temperature for 16 hours. The solution was diluted with DCM and washed with saturated NaHCO₃ solution, water and saturated brine solution. The organic layer was dried over sodium sulfate, filtered, and then concentrated under reduced pressure. The residue was purified by silical gel chromatography to give the title product (0.65 g, yield: 38%); MS (m/z): [M+H]⁺ calcd for C₃₈H₄₈FNO₁₀, 698.33; found, 642.3 (M+H−56). ¹H NMR (400 MHz, Chloroform-d) δ 7.25-7.10 (m, 5H), 6.34 (dd, J=10.1, 2.0 Hz, 1H), 6.13 (t, J=1.8 Hz, 1H), 5.13 (d, J=17.7 Hz, 1H), 5.02 (d, J=4.9 Hz, 1H), 4.90 (d, J=17.7 Hz, 1H), 4.57 (s, 1H), 4.47-4.35 (m, 1H), 3.36 (d, J=7.5 Hz, 2H), 2.80 (t, J=7.0 Hz, 2H), 2.63 (td, J=13.6, 5.9 Hz, 1H), 2.55-2.30 (m, 3H), 2.18-2.02 (m, 2H), 1.92-1.82 (m, 1H), 1.75-1.60 (m, 4H), 1.53 (s, 3H), 1.48-1.39 (m, 12H), 1.23 (s, 3H), 0.95 (s, 3H).

### Step 2: Preparation of 4-(2-aminoethyl)phenyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

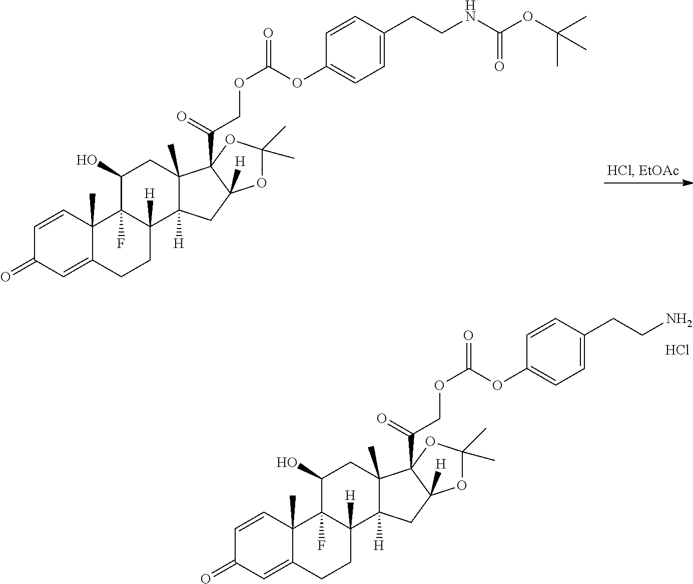

To a stirred solution of tert-butyl (4-(((2-((6αS,6βR,7S,8αS,8βS,11αR,12αS,12βS)-6β-fluoro-7-hydroxy-6α,8α,10,10-tetramethyl-4-oxo-1,2,4,6α,6,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)phenethyl)carbamate (488 mg, 0.7 mmol) in EtOAc (20 mL) was slowly added 4M HCl in ethyl acetate (5 mL) at ice-bath. The reaction mixture was allowed to cool to room temperature and then was stirred at room temperature for 16 hours. The solution was diluted with EtOAc and concentrated under reduced pressure to afford the title compound as a white solid (0.4 g, yield: 95%) that was used without further purification. MS (m/z): [M+H]⁺ calcd for C₃₃H₄₀FNO₈, 598.27; found, 598.3.

### Step 3: Preparation of Conjugate of HA and 4-(2-aminoethyl)phenyl(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

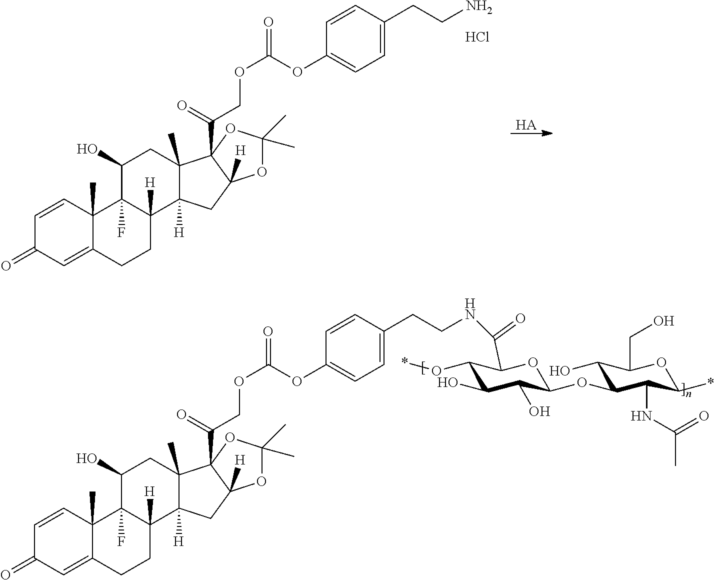

Hyaluronic acid (201 mg, 0.5 mmol carboxylic acid) was dissolved in 22.5 mL of deionized water in a 100 mL round-bottomed flask followed by the dropwise addition of 22.5 mL of 1,4-dioxane while stirring. To the solution was added 4-(2-aminoethyl)phenyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride (127 mg, 0.2 mmol), N-hydroxysuccinimide (46 mg, 0.4 mmol) and 1-ethyl-3-(3-dimethylaminopropyl)carbodiimide (38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (7.5 mL) was added to the reaction mixture, after stirring for 1 hour, 1M HCl solution (4.3 mL) was added. NaCl (293 mg, 5 mmol) was then added to the reaction mixture, which was stirred for 1 hour and followed by the dropwise addition of anhydrous alcohol (300 mL) while stirring at −10° C. The mixture was filtered. The filter cake was collected, washed with anhydrous alcohol, and dried in vacuo to give the title compound as a white solid. (sodium hyaluronate MW 500 KDa 0.19 g, yield: 59%, DSR=20%); ¹H NMR (400 MHz, Deuterium Oxide) δ 7.64-7.52 (m, 0.2H), 7.51-7.36 (m, 0.2H), 7.35-7.14 (m, 0.4H), 6.98-6.85 (m, 0.2H), 6.55-6.42 (m, 0.2H), 6.35-6.21 (m, 0.2H), 5.53-5.41 (m, 0.2H), 5.19-5.04 (m, 0.4H), 4.70-4.32 (m, 2.2H), 4.25-3.03 (m, 10.4H), 3.01-2.40 (m, 1.2H), 2.33-1.66 (m, 4.2H), 1.65-1.41 (m, 1.2H), 1.36-1.20 (m, 0.6H), 1.03-0.87 (m, 0.6H).

### Example 30

### Preparation of Conjugate of HA and (1S,2S)-2-aminocyclohexyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

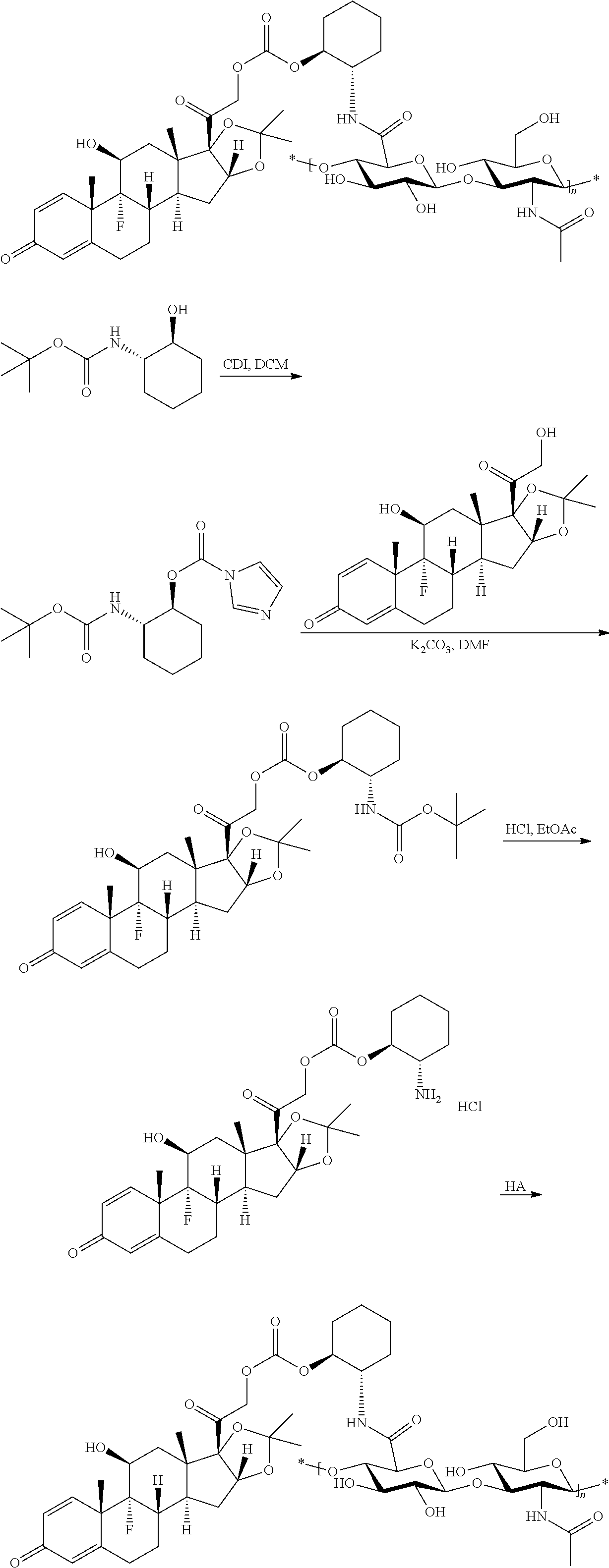

### Step 1: Preparation of (1S,2S)-2-((tert-butoxycarbonyl)amino)cyclohexyl 1H-imidazole-1-carboxylate

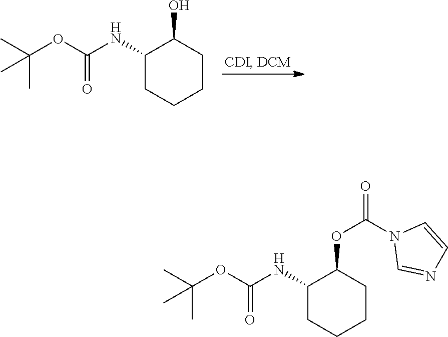 [SMILES: CC(C)(C)OC(=O)N[C@H]1CCCC[C@@H]1O ; CC(C)(C)OC(=O)N[C@H]1CCCC[C@@H]1OC(=O)n1ccnc1]

To a solution of tert-butyl ((1S,2S)-2-hydroxycyclohexyl)carbamate (215 mg, 1 mmol) in DCM (10 mL) was added 1,1′-carbonyldiimidazole (324 mg, 2 mmol). The resulting mixture was stirred at room temperature for 19 hours. The solution was diluted with DCM and washed with water. The organic layer was dried over sodium sulfate, filtered, and then concentrated under reduced pressure to afford the title compound as a white solid (0.3 g, yield: 48%) that was used without further purification. MS (m/z): [M+H]⁺ calcd for C₁₅H₂₃N₃O₄, 310.17; found, 310.2

### Step 2: Preparation of tert-butyl ((1S,2S)-2-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)cyclohexyl)carbamate

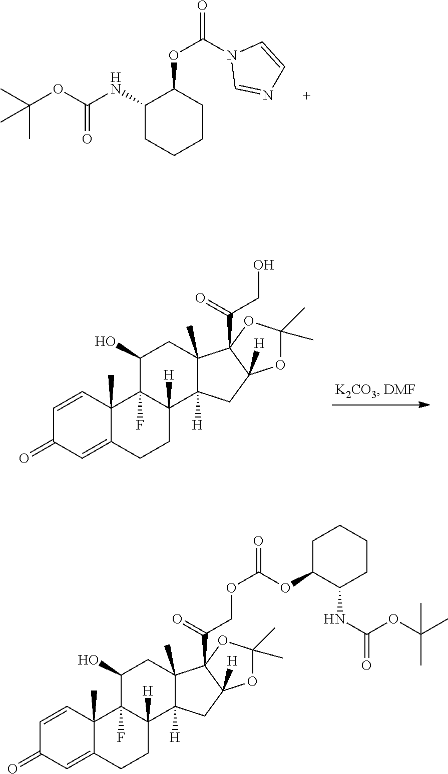

To a solution of 9α-fluoro-11β,16α,17,21-tetrahydroxy-1,4-pregnadiene-3,20-dione (435 mg, 1 mmol) in DMF (10 mL) was added (1S,2S)-2-((tert-butoxycarbonyl)amino)cyclohexyl 1H-imidazole-1-carboxylate (309 mg, 1 mmol) and K₂CO₃ (138 mg, 1 mmol) under N₂, the resulting mixture was stirred at room temperature for 19 hours. The reaction mixture was poured into water (40 mL) at room temperature, filtered and the filter cake was dissolved in DCM (40 mL). The solution was dried over sodium sulfate, filtered and then concentrated under reduced pressure. The residue was purified by silical gel chromatography to give the title product (0.3 g, yield: 44%); MS (m/z): [M+H]⁺ calcd for C₃₆H₅₀FNO₁₀, 676.34; found, 620.4 (M+H−56). ¹H NMR (400 MHz, Chloroform-d) δ 7.24 (d, J=10.3 Hz, 1H), 6.33 (dd, J=10.1, 1.9 Hz, 1H), 6.12 (d, J=1.8 Hz, 1H), 5.09-4.95 (m, 2H), 4.76 (d, J=17.7 Hz, 1H), 4.64 (s, 1H), 4.51-4.38 (m, 2H), 3.59 (s, 1H), 2.81-2.75 (m, 1H), 2.69-2.57 (m, 1H), 2.54-2.29 (m, 3H), 2.15-2.01 (m, 3H), 1.91-1.74 (s, 2H), 1.72-1.51 (m, 8H), 1.48-1.40 (m, 12H), 1.38-1.28 (m, 4H), 1.20 (s, 3H), 0.94 (s, 3H).

### Step 3: Preparation of (1S,2S)-2-aminocyclohexyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

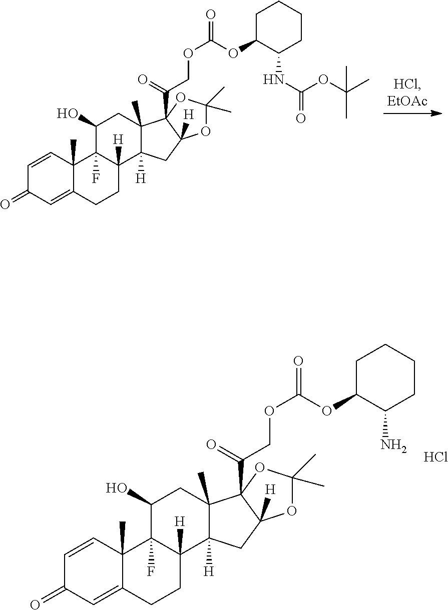

To a stirred solution of tert-butyl ((1S,2S)-2-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)cyclohexyl)carbamate (300 mg, 0.44 mmol) in EtOAc (10 mL) was slowly added 4M HCl in ethyl acetate (2 mL) at ice-bath. The reaction mixture was allowed to cool to room temperature and then was stirred at room temperature for 21 hours. The solution was diluted with EtOAc and concentrated under reduced pressure to afford the title compound as a white solid (0.26 g, yield: 96%) that was used without further purification. MS (m/z): [M+H]⁺ calcd for C₃₁H₄₂FNO₈, 576.29; found, 576.2.

### Step 4: Preparation of (1S,2S)-2-aminocyclohexyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

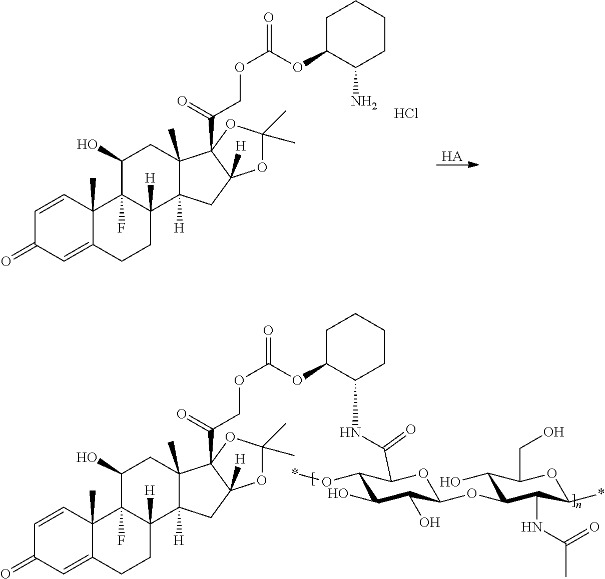

Hyaluronic acid (202 mg, 0.5 mmol carboxylic acid) was dissolved in 22.5 mL of deionized water in a 100 mL round-bottomed flask followed by the dropwise addition of 22.5 mL of 1,4-dioxane while stirring. To the solution was added (1S,2S)-2-aminocyclohexyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride (122 mg, 0.2 mmol), N-hydroxysuccinimide (46 mg, 0.4 mmol) and 1-ethyl-3-(3-dimethylaminopropyl)carbodiimide (38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (7.5 mL) was added to the reaction mixture, after stirring for 1 hour, 1M HCl solution (4.3 mL) was added. NaCl (293 mg, 5 mmol) was then added to the reaction mixture, which was stirred for 1 hour and followed by the dropwise addition of anhydrous alcohol (225 mL) while stirring at −10° C. The mixture was filtered. The filter cake was collected, washed with anhydrous alcohol and dried in vacuo to give the title compound as a white solid. (sodium hyaluronate MW 500 KDa 0.195 g, yield: 61%, DSR=11%); ¹H NMR (400 MHz, Deuterium Oxide) δ 7.67-7.53 (m, 0.11H), 6.58-6.43 (m, 0.11H), 6.33-6.21 (m, 0.11H), 5.39-5.24 (m, 0.11H), 5.15-4.91 (m, 0.22H), 4.74-4.33 (m, 2.22H), 4.32-3.01 (m, 10.11H), 2.95-2.41 (m, 0.55H), 2.40-1.66 (m, 3.99H), 1.65-1.47 (m, 0.66H), 1.46-1.25 (m, 0.77H), 1.03-0.87 (m, 0.33H).

### Example 31

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (7-azaspiro[3.5]nonan-2-yl) carbonate

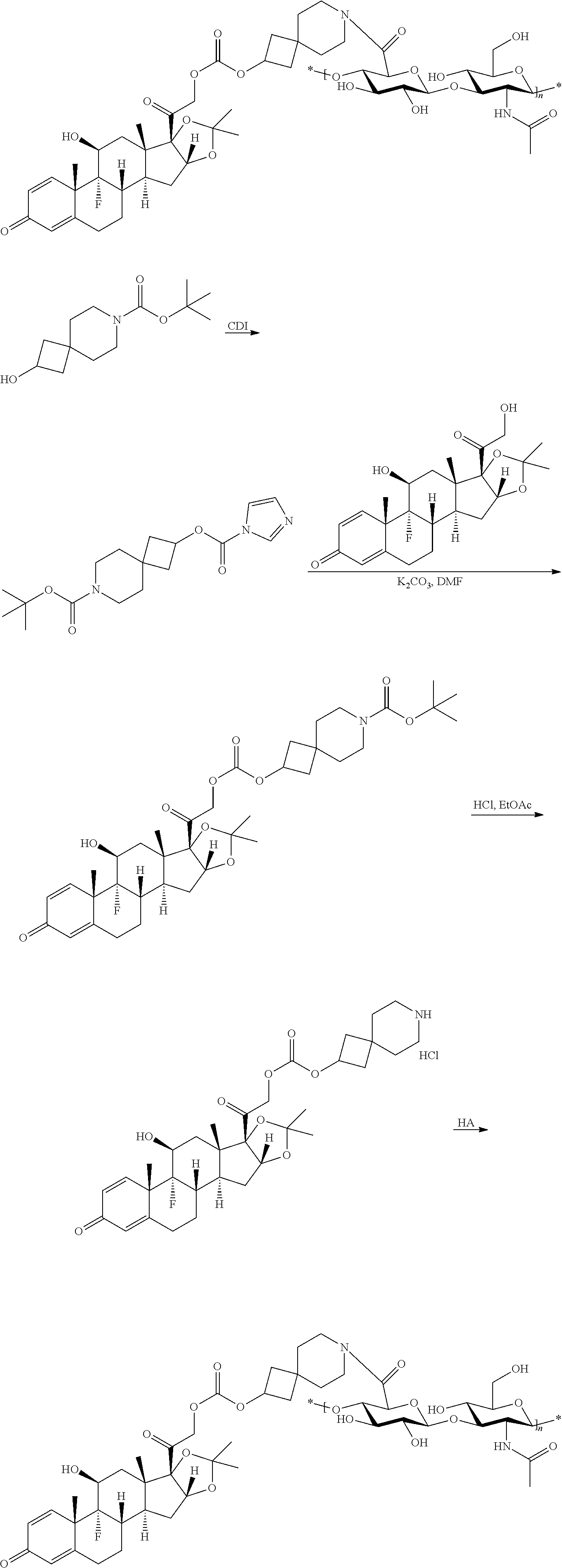

### Step 1: Preparation of tert-butyl 2-((1H-imidazole-1-carbonyl)oxy)-7-azaspiro[3.5]nonane-7-carboxylate

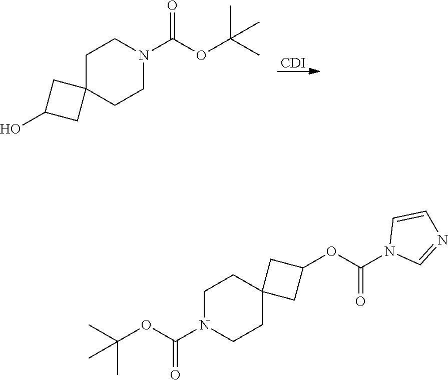

To a solution of tert-butyl 2-hydroxy-7-azaspiro[3.5]nonane-7-carboxylate (362 mg, 1.5 mmol) in DCM (10 mL) was added 1,1′-carbonyldiimidazole (486 mg, 3 mmol). The resulting mixture was stirred at room temperature for 18 hours. The solution was diluted with DCM and washed with water. The organic layer was dried over sodium sulfate, filtered, and then concentrated under reduced pressure to afford the title compound as a yellow oil (0.5 g, yield: 99%) that was used without further purification. MS (m/z): [M+H]⁺ calcd for C₁₇H₂₅N₃O₄, 336.18; found, 336.3

### Step 2: Preparation of tert-butyl 2-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)-7-azaspiro[3.5]nonane-7-carboxylate

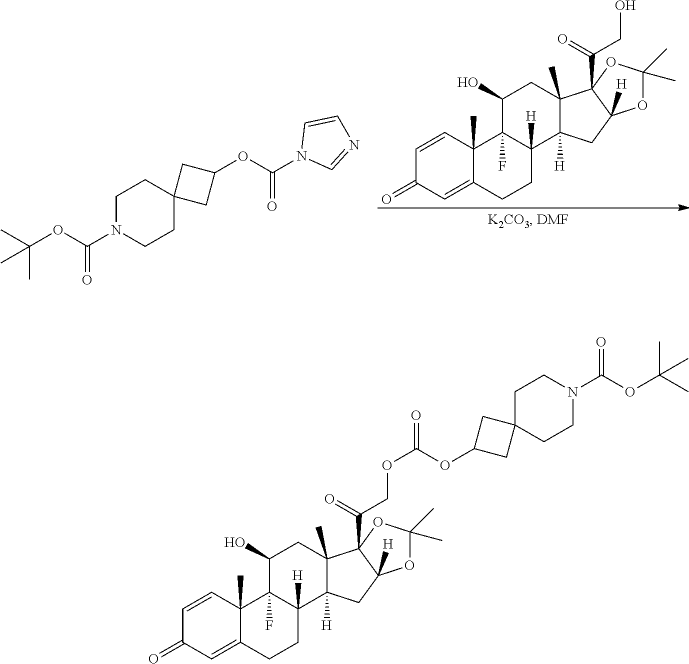

To a solution of 9α-fluoro-11β,16α,17,21-tetrahydroxy-1,4-pregnadiene-3,20-dione (652 mg, 1.5 mmol) in DMF (15 mL) was added tert-butyl 2-((1H-imidazole-1-carbonyl)oxy)-7-azaspiro[3.5]nonane-7-carboxylate (503 mg, 1.5 mmol) and K₂CO₃ (207 mg, 1.5 mmol) under N₂, The resulting mixture was stirred at room temperature for 3 hours. The reaction mixture was poured into water (45 mL) at room temperature, filtered and the filter cake was dissolved in DCM (40 mL). The solution was dried over sodium sulfate, filtered and then concentrated under reduced pressure. The residue was purified by silical gel chromatography to give the title product (0.27 g, yield: 25%); MS (m/z): [M+H]⁺ calcd for C₃₈H₅₂FNO₁₀, 702.36; found, 646.3 (M+H−56)). ¹H NMR (400 MHz, Chloroform-d) δ 7.19 (d, J=10.2 Hz, 1H), 6.34 (dd, J=10.2, 1.9 Hz, 1H), 6.13 (t, J=1.7 Hz, 1H), 5.13-4.92 (m, 3H), 4.77 (d, J=17.8 Hz, 1H), 4.42 (dt, J=9.2, 2.9 Hz, 1H), 3.44-3.22 (m, 4H), 2.71-2.56 (m, 1H), 2.55-2.27 (m, 5H), 2.15-2.04 (m, 2H), 2.01-1.93 (m, 2H), 1.91-1.83 (m, 1H), 1.73-1.66 (m, 4H), 1.65-1.51 (m, 7H), 1.45 (s, 9H), 1.43 (s, 3H), 1.21 (s, 3H), 0.94 (s, 3H).

### Step 3: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (7-azaspiro[3.5]nonan-2-yl) carbonate

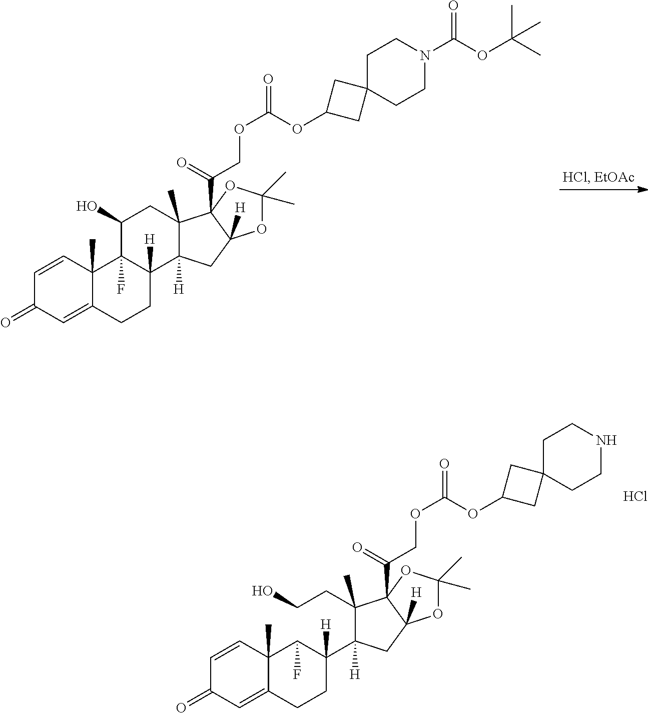

To a stirred solution of tert-butyl 2-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)-7-azaspiro[3.5]nonane-7-carboxylate (270 mg, 0.38 mmol) in EtOAc (10.8 mL) was slowly added 4M HCl in ethyl acetate (2.2 mL) at ice-bath. The reaction mixture was allowed to cool to room temperature and then was stirred at room temperature for 21 hours. The solution was diluted with EtOAc and concentrated under reduced pressure to afford the title compound as a white solid (0.23 g, yield: 95%) that was used without further purification. MS (m/z): [M+H]⁺ calcd for C₃₃H₄₄FNO₈, 602.31; found, 602.4.

### Step 4: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (7-azaspiro[3.5]nonan-2-yl) carbonate

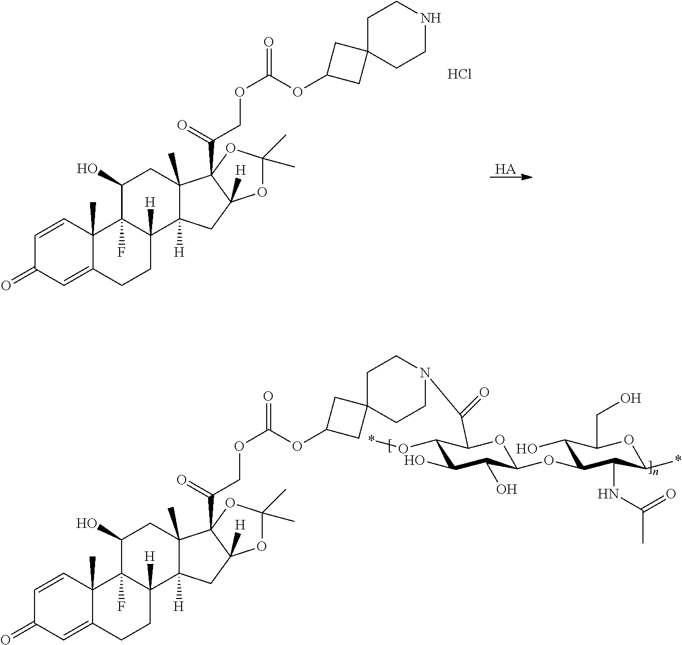

Hyaluronic acid (161 mg, 0.4 mmol carboxylic acid) was dissolved in 17.8 mL of deionized water in a 100 mL round-bottomed flask followed by the dropwise addition of 17.8 mL of 1,4-dioxane while stirring. To the solution was added 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (7-azaspiro[3.5]nonan-2-yl) carbonate hydrochloride (102 mg, 0.16 mmol), N-hydroxysuccinimide (37 mg, 0.32 mmol) and 1-ethyl-3-(3-dimethylaminopropyl)carbodiimide (31 mg, 0.16 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (2 mL) was added to the reaction mixture, after stirring for 1 hour, 1M HCl solution (1 mL) was added. NaCl (293 mg, 5 mmol) was then added to the reaction mixture, which was stirred for 1 hour and followed by the dropwise addition of anhydrous alcohol (250 mL) while stirring at −10° C. The mixture was filtered. The filter cake was collected, washed with anhydrous alcohol and dried in vacuo to give the title compound as a white solid. (sodium hyaluronate MW 500 KDa 0.129 g, yield: 50%, DSR=6%); ¹H NMR (400 MHz, Deuterium Oxide) δ 7.63-7.52 (m, 0.06H), 6.51-6.44 (m, 0.06H), 6.31-6.24 (m, 0.06H), 5.37-5.26 (m, 0.06H), 5.15-4.94 (m, 0.12H), 4.74-4.31 (m, 2.06H), 4.29-3.02 (m, 10.24H), 2.88-2.42 (m, 0.36H), 2.32-1.64 (m, 3.3H), 1.63-1.46 (m, 0.84H), 1.34-1.24 (m, 0.18H), 1.02-0.88 (m, 0.18H).

### Example 32

### Preparation of Conjugate of HA and 2-(2-aminoethoxy)ethyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

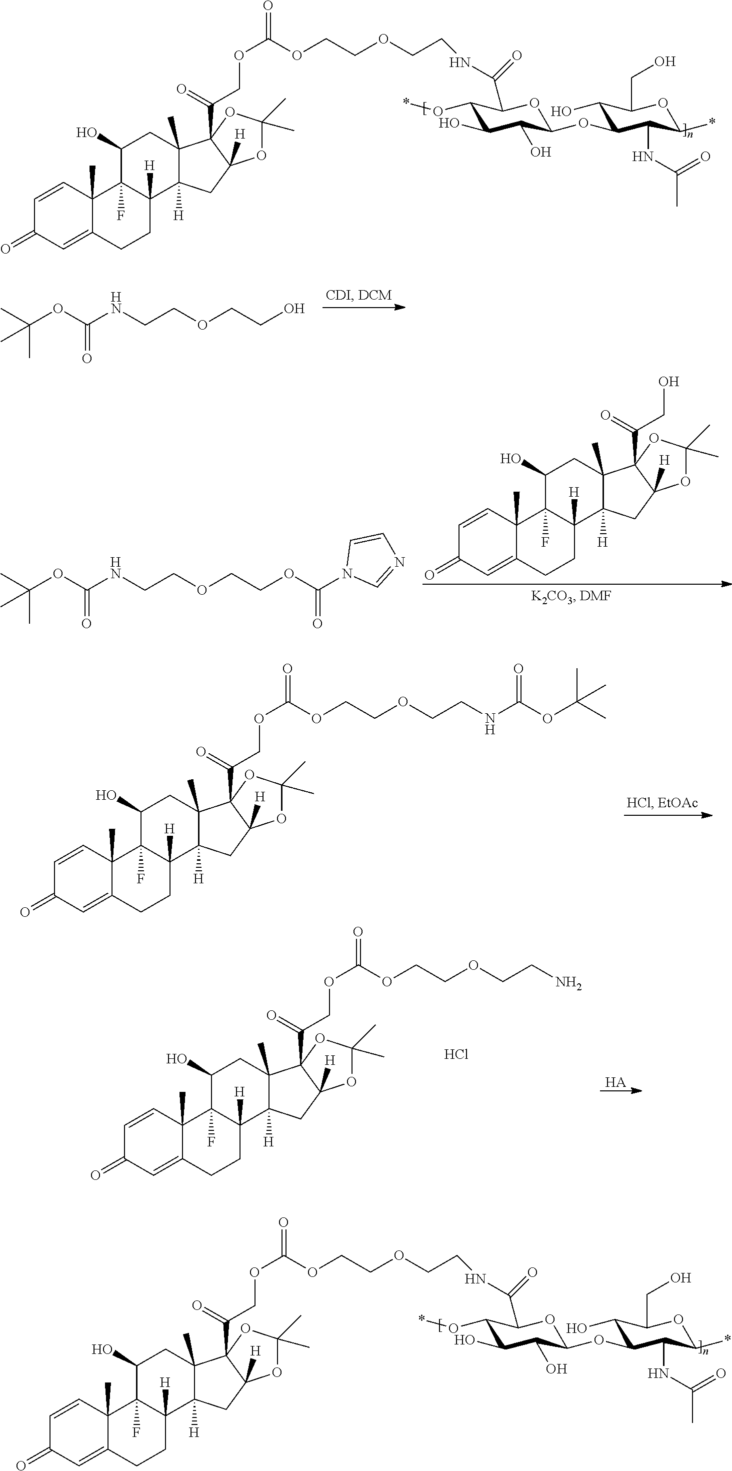

### Step 1: Preparation of 2-(2-((tert-butoxycarbonyl)amino)ethoxy)ethyl 1-imidazole-1-carboxylate

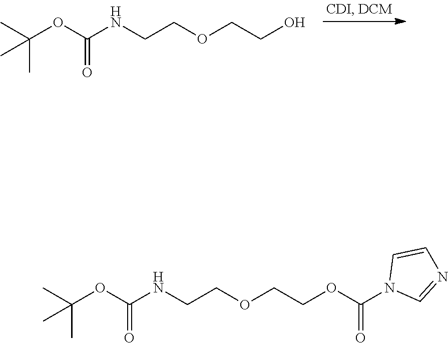 [SMILES: CC(C)(C)OC(=O)NCCOCCO ; CC(C)(C)OC(=O)NCCOCCOC(=O)n1ccnc1]

To a solution of tert-butyl (2-(2-hydroxyethoxy)ethyl)carbamate (308 mg, 1.5 mmol) in DCM (10 mL) was added 1,1′-carbonyldiimidazole (486 mg, 3 mmol). The resulting mixture was stirred at room temperature for 18 hours. The solution was diluted with DCM and washed with water. The organic layer was dried over sodium sulfate, filtered, and then concentrated under reduced pressure to afford the title compound as a yellow oil (0.44 g, yield: 98%) that was used without further purification. MS (m/z): [M+H]⁺ calcd for C₁₃H₂₁N₃O₅, 300.15; found, 300.2

### Step 2: Preparation of tert-butyl (2-(2-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)ethoxy)ethyl)carbamate

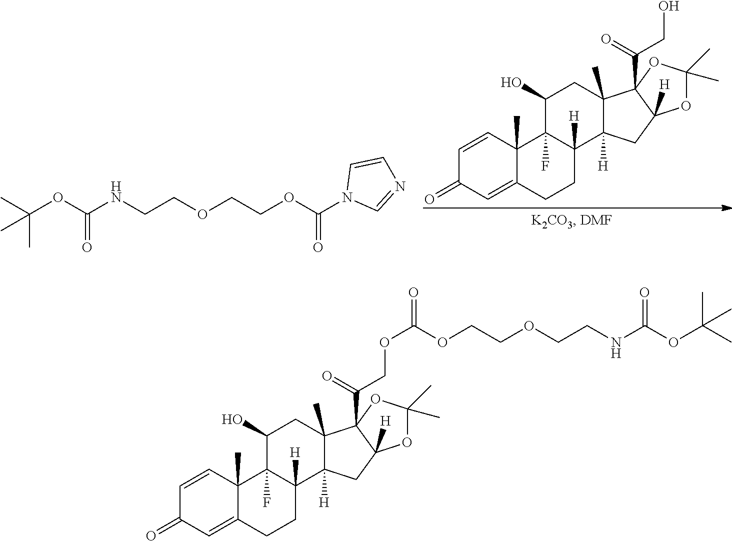

To a solution of 9α-fluoro-11β,16α,17,21-tetrahydroxy-1,4-pregnadiene-3,20-dione (652 mg, 1.5 mmol) in DMF (15 mL) was added 2-(2-((tert-butoxycarbonyl)amino)ethoxy)ethyl 1H-imidazole-1-carboxylate (449 mg, 1.5 mmol) and K₂CO₃ (207 mg, 1.5 mmol) under N₂, the resulting mixture was stirred at room temperature for 3 hours. The reaction mixture was poured into water (45 mL) at room temperature, filtered and the filter cake was dissolved in DCM (40 mL). The solution was dried over sodium sulfate, filtered and then concentrated under reduced pressure. The residue was purified by silical gel chromatography to give the title product (0.36 g, yield: 36%); MS (m/z): [M+H]⁺ calcd for C₃₄H₄₈FNO₁₁, 666.32; found, 610.3 (M+H−56). ¹H NMR (400 MHz, Chloroform-d) δ 7.20 (d, J=10.1 Hz, 1H), 6.34 (dd, J=10.1, 1.9 Hz, 1H), 6.13 (t, J=1.8 Hz, 1H), 4.92 (dd, J=71.2, 11.0 Hz, 4H), 4.38 (d, J=30.1 Hz, 2H), 3.72 (s, 2H), 3.56 (dt, J=5.5, 3.8 Hz, 2H), 3.33 (s, 2H), 2.70-2.57 (m, 1H), 2.55-2.34 (m, 3H), 2.19-2.07 (m, 1H), 1.93-1.78 (m, 1H), 1.73-1.59 (m, 6H), 1.56 (s, 3H), 1.46 (s, 9H), 1.43 (s, 3H), 1.21 (s, 3H), 0.97 (s, 3H).

### Step 3: Preparation of 2-(2-aminoethoxy)ethyl(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

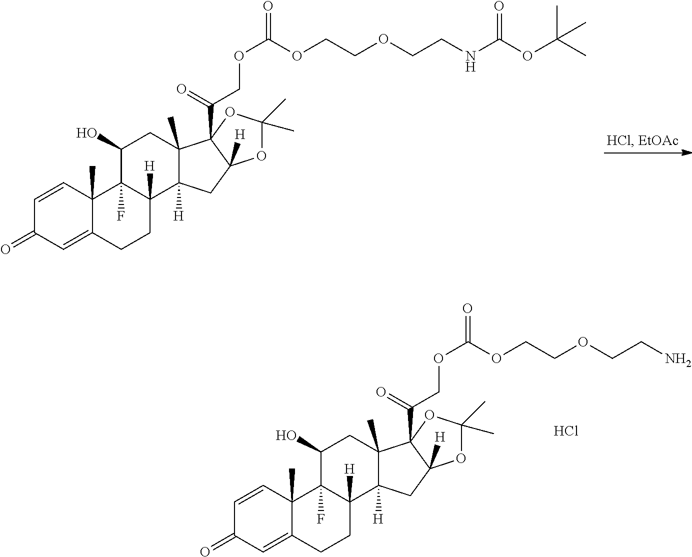

To a stirred solution of tert-butyl (2-(2-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)ethoxy)ethyl)carbamate (360 mg, 0.54 mmol) in EtOAc (14.4 mL) was slowly added 4M HCl in ethyl acetate (2.9 mL) at ice-bath. The reaction mixture was allowed to cool to room temperature and then was stirred at room temperature for 20 hours. The solution was diluted with EtOAc and concentrated under reduced pressure to afford the title compound as a white solid (0.3 g, yield: 92%) that was used without further purification. MS (m/z): [M+H]⁺ calcd for C₂₉H₄₀FNO₉, 566.27; found, 566.4.

### Step 4: Preparation of Conjugate of HA and 2-(2-aminoethoxy)ethyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

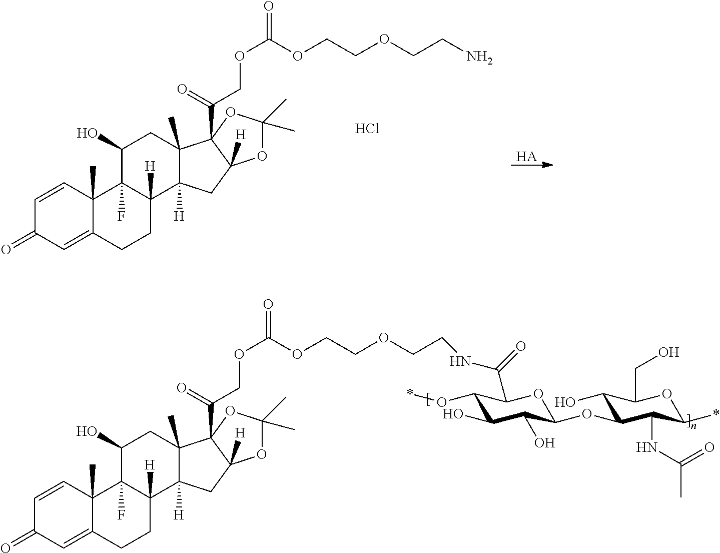

Hyaluronic acid (161 mg, 0.4 mmol carboxylic acid) was dissolved in 17.8 mL of deionized water in a 100 mL round-bottomed flask followed by the dropwise addition of 17.8 mL of 1,4-dioxane while stirring. To the solution was added 2-(2-aminoethoxy)ethyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride (96 mg, 0.16 mmol), N-hydroxysuccinimide (37 mg, 0.32 mmol) and 1-ethyl-3-(3-dimethylaminopropyl)carbodiimide (31 mg, 0.16 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (2 mL) was added to the reaction mixture, after stirring for 1 hour, 1M HCl solution (1 mL) was added. NaCl (293 mg, 5 mmol) was then added to the reaction mixture, which was stirred for 1 hour and followed by the dropwise addition of anhydrous alcohol (250 mL) while stirring at −10° C. The mixture was filtered. The filter cake was collected, washed with anhydrous alcohol and dried in vacuo to give the title compound as a white solid. (sodium hyaluronate MW 500 KDa 0.13 g, yield: 51%, DSR=8%); ¹H NMR (400 MHz, Deuterium Oxide) δ 7.63-7.50 (m, 0.08H), 6.52-6.43 (m, 0.08H), 6.33-6.21 (m, 0.08H), 5.40-5.28 (m, 0.08H), 5.17-4.94 (m, 0.16H), 4.73-4.34 (m, 2.16H), 4.19-2.98 (m, 10.48H), 2.85-2.42 (m, 0.32H), 2.33-1.65 (m, 3.16H), 1.64-1.42 (m, 0.88H), 1.35-1.24 (m, 0.24H), 1.02-0.87 (m, 0.24H).

### Example 33

### Preparation of Conjugate of HA and methyl (S)-2-amino-3-(4-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)phenyl)propanoate

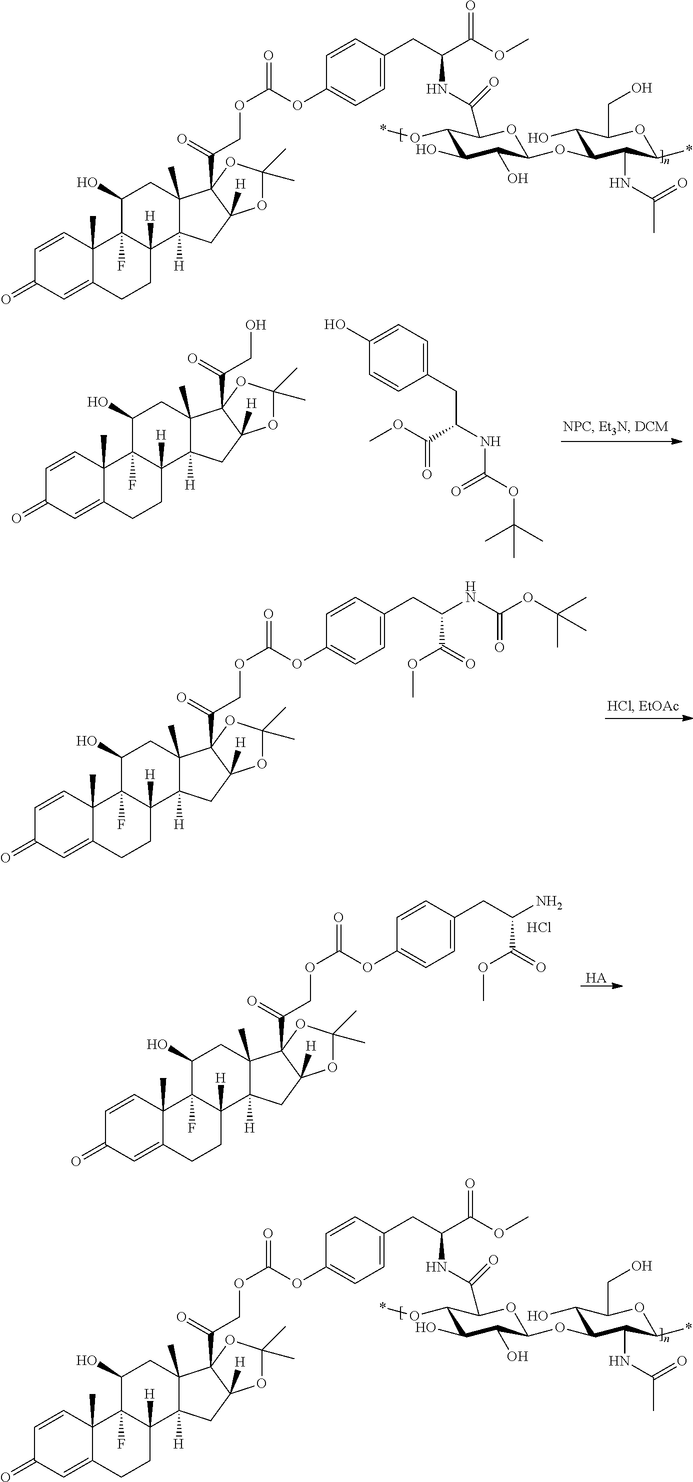

### Step 1: Preparation of methyl (S)-2-((tert-butoxycarbonyl)amino)-3-(4-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)phenyl)propanoate

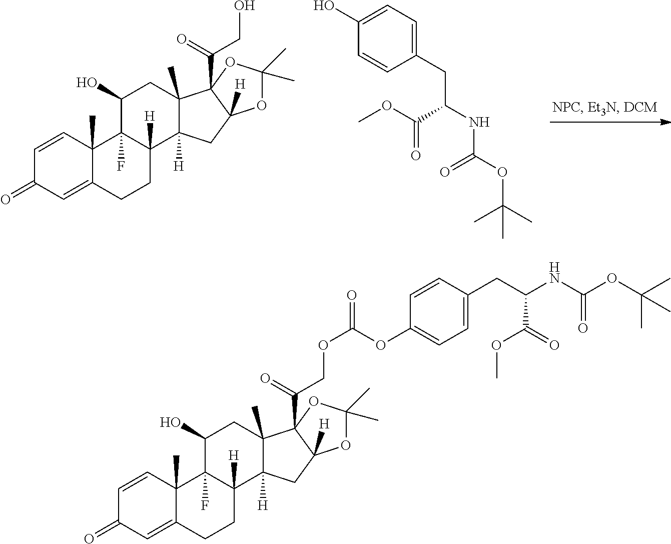

To a mixture of 9α-fluoro-11β,16α,17,21-tetrahydroxy-1,4-pregnadiene-3,20-dione (694 mg, 1.6 mmol), and bis (4-nitrophenyl) carbonate (730 mg, 2.4 mmol) in dichloromethane (20 mL) was added triethylamine (0.7 mL, 5 mmol) under N₂, the reaction mixture was heated to reflux for 18 hours. Then methyl (tert-butoxycarbonyl)-L-tyrosinate (590 mg, 2 mmol) was added and the resulting mixture was stirred at room temperature for 7 hours. The solution was diluted with DCM and washed with saturated NaHCO₃ solution, water and saturated brine solution. The organic layer was dried over sodium sulfate, filtered, and then concentrated under reduced pressure. The residue was purified by silical gel chromatography to give the title product (0.4 g, yield: 33%); MS (m/z): [M+H]⁺ calcd for C₄₀H₅₀FNO₁₂, 756.33; found, 700.3 (M+H−56). ¹H NMR (400 MHz, Chloroform-d) δ 7.23-7.09 (m, 5H), 6.34 (dd, J=10.1, 1.9 Hz, 1H), 6.13 (t, J=1.7 Hz, 1H), 5.11 (d, J=17.6 Hz, 1H), 5.02 (d, J=4.9 Hz, 2H), 4.91 (d, J=17.6 Hz, 1H), 4.63-4.52 (m, 1H), 4.45-4.35 (m, 1H), 3.73 (s, 3H), 3.20-2.95 (m, 2H), 2.69-2.56 (m, 1H), 2.55-2.30 (m, 3H), 2.20-2.07 (m, 2H), 1.93-1.82 (m, 1H), 1.75-1.56 (m, 4H), 1.54 (s, 3H), 1.47-1.36 (m, 12H), 1.23 (s, 3H), 0.95 (s, 3H).

### Step 2: Preparation of methyl (S)-2-amino-3-(4-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)phenyl)propanoate

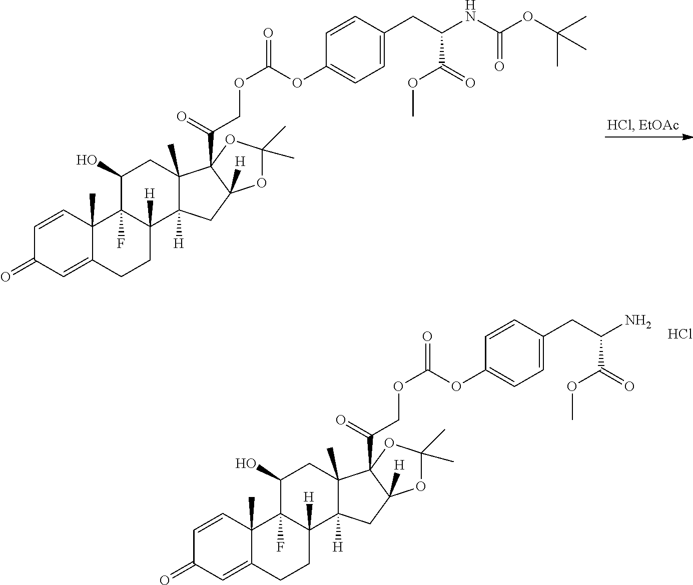

To a stirred solution of methyl (S)-2-((tert-butoxycarbonyl)amino)-3-(4-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)phenyl)propanoate (378 mg, 0.5 mmol) in EtOAc (15 mL) was slowly added 4M HCl in ethyl acetate (3 mL) at ice-bath. The reaction mixture was allowed to cool to room temperature and then was stirred at room temperature for 20 hours. The solution was diluted with EtOAc and concentrated under reduced pressure to afford the title compound as a white solid (0.34 g, yield: 98%) that was used without further purification. MS (m/z): [M+H]⁺ calcd for C₃₅H₄₂FNO₁₀, 656.28; found, 656.3.

### Step 3: Preparation of Conjugate of HA and methyl (S)-2-amino-3-(4-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)phenyl)propanoate

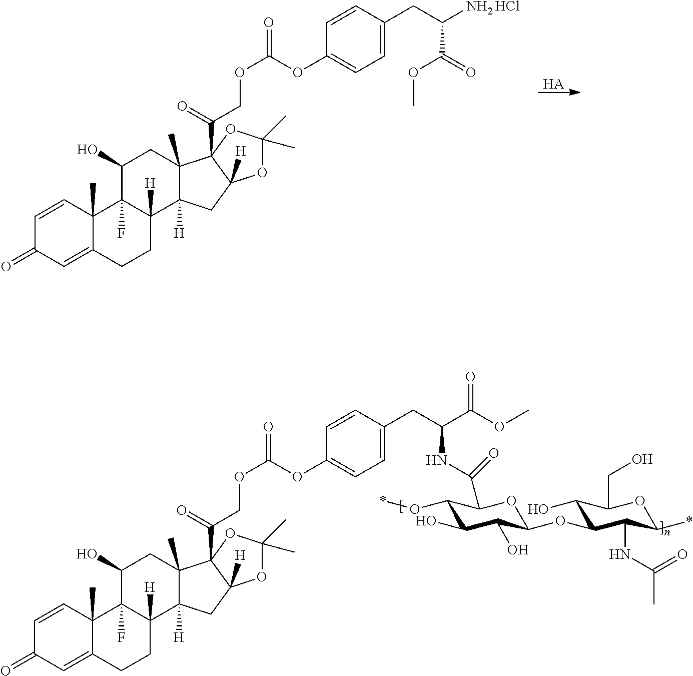

Hyaluronic acid (201 mg, 0.5 mmol carboxylic acid) was dissolved in 22.5 mL of deionized water in a 100 mL round-bottomed flask followed by the dropwise addition of 22.5 mL of 1,4-dioxane while stirring. To the solution was added methyl (S)-2-amino-3-(4-(((2-((6αS,6βR,7S,8αS,8βS,11αR,12αS,120 S)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethoxy)carbonyl)oxy)phenyl)propanoate hydrochloride (138 mg, 0.2 mmol), N-hydroxysuccinimide (46 mg, 0.4 mmol) and 1-ethyl-3-(3-dimethylaminopropyl)carbodiimide (38 mg, 0.2 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (7.5 mL) was added to the reaction mixture, after stirring for 1 hour, 1M HCl solution (4.3 mL) was added. NaCl (293 mg, 5 mmol) was then added to the reaction mixture, which was stirred for 1 hour and followed by the dropwise addition of anhydrous alcohol (300 mL) while stirring at −10° C. The mixture was filtered. The filter cake was collected, washed with anhydrous alcohol and dried in vacuo to give the title compound as a white solid. (sodium hyaluronate MW 500 KDa 0.19 g, yield: 57%, DSR=30%); ¹H NMR (400 MHz, Deuterium Oxide) δ 7.61-7.50 (m, 0.3H), 7.47-7.14 (m, 1.2H), 6.51-6.42 (m, 0.3H), 6.32-6.20 (m, 0.3H), 5.51-5.34 (m, 0.3H), 5.17-5.02 (m, 0.9H), 4.72-4.35 (m, 2.3H), 4.18-2.98 (m, 11.5H), 2.82-2.39 (m, 1.2H), 2.30-1.64 (m, 4.8H), 1.63-1.41 (m, 1.8H), 1.34-1.23 (m, 0.9H), 1.02-0.82 (m, 0.9H).

### Example 34

### Preparation of Conjugate of HA and 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate

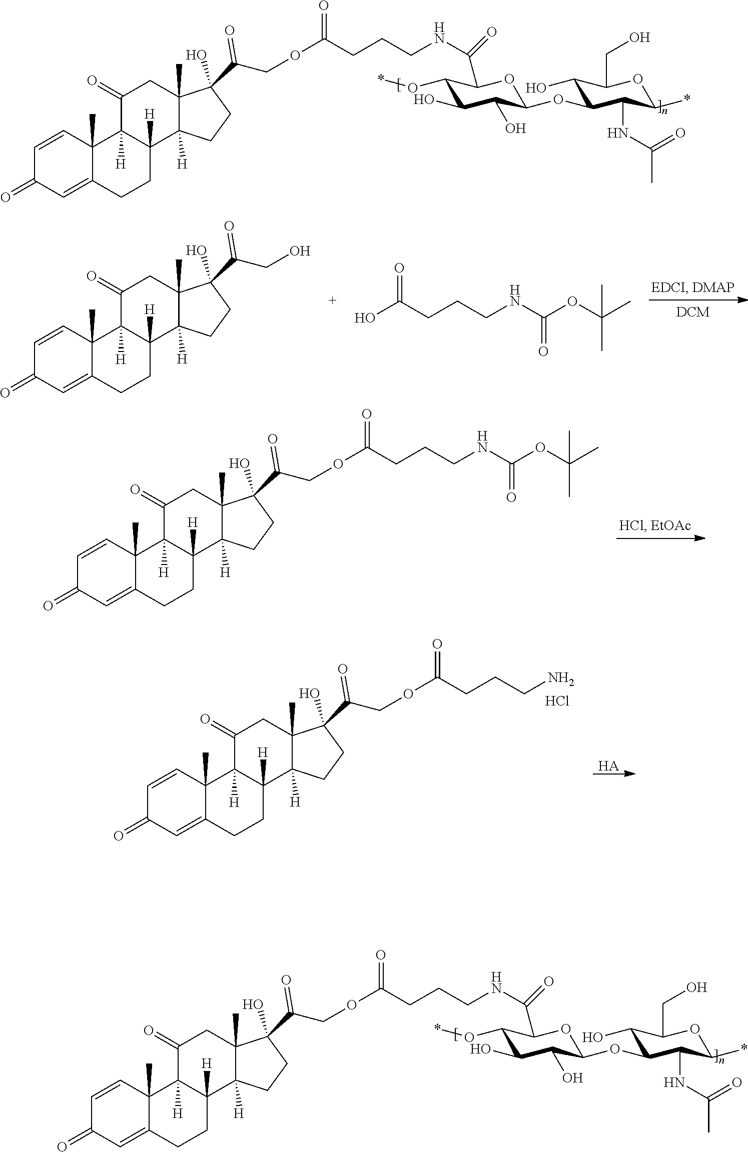

### Step 1: Preparation of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-((tert-butoxycarbonyl)amino)butanoate

The title compound was prepared in analogy to example 2, step 1 by using (8S,9S,10R,13S,14S,17R)-17-hydroxy-17-(2-hydroxyacetyl)-10,13-dimethyl-7,8,9,10,12,13,14,15,16,17-decahydro-3H-cyclopenta[a]phenanthrene-3,11(6H)-dione instead of 9α-fluoro-11β,16α,17,21-tetrahydroxy-1,4-pregnadiene-3,20-dione was obtained as a white solid (0.274 g, yield: 50.4%). MS (m/z): [M+H]⁺ calcd for C₃₀H₄₁NO₈, 544.28; found, 566.2 [M+Na]⁺.

### Step 2: Preparation of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate hydrochloride

The title compound was prepared in analogy to example 2, step 2 by using 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-((tert-butoxycarbonyl)amino)butanoate instead of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-((tert-butoxycarbonyl)amino)butanoate was obtained as a white solid (0.21 g, yield: 98%). MS (m/z): [M+H]⁺ calcd for C₂₅H₃₃NO₆, 444.23; found, 444.2. ¹H NMR (400 MHz, Deuterium Oxide) δ 7.88 (d, J=10.2 Hz, 1H), 6.32 (dd, J=10.2, 2.0 Hz, 1H), 6.24 (t, J=1.6 Hz, 1H), 5.15 (d, J=18.1 Hz, 1H), 5.03 (d, J=18.1 Hz, 1H), 3.12 (t, J=7.8 Hz, 2H), 2.94 (d, J=12.1 Hz, 1H), 2.72-2.64 (m, 3H), 2.58-2.48 (m, 1H), 2.46-2.15 (m, 5H), 2.14-1.90 (m, 3H), 1.83 (ddd, J=15.1, 9.5, 5.6 Hz, 1H), 1.58 (qd, J=12.1, 5.7 Hz, 1H), 1.48 (s, 3H), 1.42-1.20 (m, 2H), 0.69 (s, 3H).

### Step 3: Preparation of Conjugate of HA and 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate

Hyaluronic acid (227 mg, 0.564 mmol carboxylic acid) was dissolved in 25 mL of deionized water in a 100 mL round-bottomed flask followed by the dropwise addition of 25 mL of 1,4-dioxane while stirring. To the solution was added 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate hydrochloride (100 mg, 0.226 mmol), N-Hydroxysuccinimide (452 mg, 0.451 mmol) and 1-ethyl-3-(3-dimethylaminopropyl)carbodiimide (45 mg, 0.226 mmol) causing the viscosity to increase temporarily. The pH of the reaction mixture was adjusted to 6˜6.5 with 5% NaHCO₃ solution. Then the reaction mixture was stirred for 24 hours at room temperature. 5% NaHCO₃ solution (2 mL) was added to the reaction mixture, after stirring for 1 hour, 1M HCl solution (1 mL) was added. NaCl (340 mg) was then added to the reaction mixture, which was stirred for 1 hour and followed by the dropwise addition of anhydrous alcohol (300 mL) while stirring at −10° C. The mixture was filtered. The filter cake was collected, washed with anhydrous alcohol and dried in vacuo to give the title compound as a white solid. (sodium hyaluronate MW 500 KDa 0.255 g, yield: 83.9%, DSR=17%). ¹H NMR (400 MHz, D₂O) δ 7.89 (d, J=9.8 Hz, 0.17H), 6.32 (d, J=10.1 Hz, 0.17H), 6.23 (s, 0.17H), 5.20-4.91 (m, 0.34H), 4.68-4.33 (m, 2H), 4.27-3.05 (m, 10H), 2.95-2.83 (m, 0.34H), 2.72-2.14 (m, 1.7H), 2.13-1.67 (m, 3.68H), 1.66-1.26 (m, 1.02H), 0.69 (s, 0.51H).

With this procedure, reaction of sodium hyaluronate (MW 50 KDa) provided corresponding product (0.251 g, yield: 82.6%, DSR=20%). ¹H NMR (400 MHz, D₂O) δ 7.89 (d, J=9.7 Hz, 0.2H), 6.31 (d, J=10.0 Hz, 0.2H), 6.20 (s, 0.2H), 5.07 (dd, J=46.2, 18.0 Hz, 0.4H), 4.68-4.33 (m, 2H), 4.22-3.10 (m, 10H), 2.93 (d, J=11.5 Hz, 0.4H), 2.73-2.13 (m, 2H), 2.12-1.65 (m, 3.8H), 1.64-1.24 (m, 1.2H), 0.68 (s, 0.6H).

### Example 35

### Preparation of Conjugate of CS and 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate

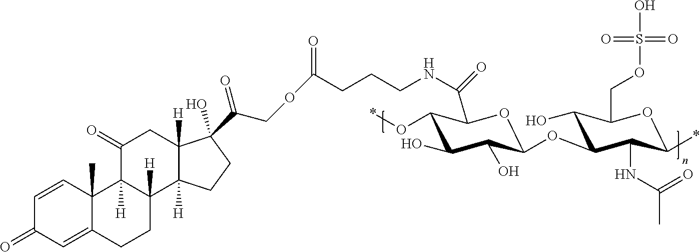

The title compound was prepared in analogy to example 34, step 3 by using CS (Chondroitin sulfate) instead of HA (Hyaluronic acid) was obtained as a white solid (0.238 g, yield: 70%, DSR=32%). ¹H NMR (400 MHz, D₂O) δ 7.89 (d, J=9.6 Hz, 0.32H), 6.31 (d, J=10.1 Hz, 0.32H), 6.23 (s, 0.32H), 5.10 (dd, J=34.0, 21.8 Hz, 0.64H), 4.69-4.38 (m, 2H), 4.31-3.13 (m, 10H), 3.04-2.82 (m, 0.64H), 2.77-2.14 (m, 3.2H), 2.13-1.75 (m, 4.28H), 1.70-1.15 (m, 1.92H), 0.68 (d, J=8.5 Hz, 0.96H).

### Example 36

### Preparation of Conjugate of HA and 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 4-aminobutanoate

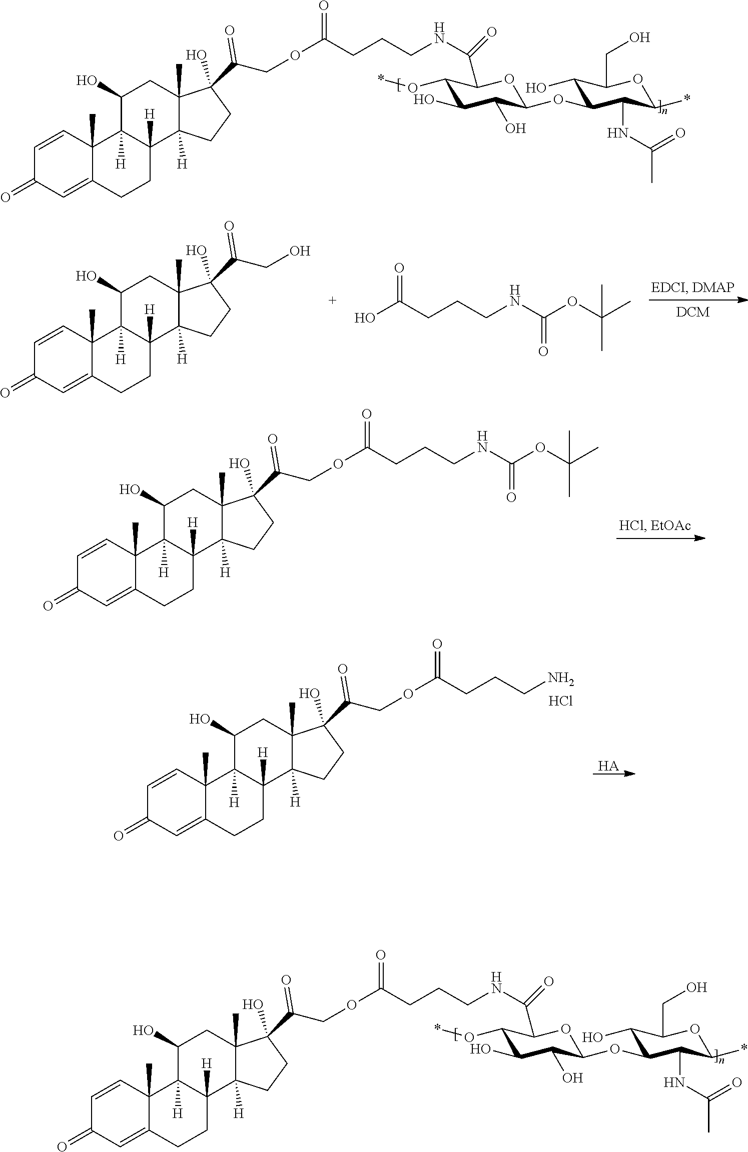

### Step 1: Preparation of 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 4-((tert-butoxycarbonyl)amino)butanoate

The title compound was prepared in analogy to example 2, step 1 by using (8S,9S,10R,11S,13S,14S,17R)-11,17-dihydroxy-17-(2-hydroxyacetyl)-10,13-dimethyl-6,7,8,9,10,11,12,13,14,15,16,17-dodecahydro-3H-cyclopenta[a]phenanthren-3-one instead of 9α-fluoro-11β,16α,17,21-tetrahydroxy-1,4-pregnadiene-3,20-dione was obtained as a white solid (0.283 g, yield: 78.6%). MS (m/z): [M+H]⁺ calcd for C₃₀H₄₃NO₈, 546.30; found, 568.2 [M+Na]⁺.

### Step 2: Preparation of 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 4-aminobutanoate hydrochloride

The title compound was prepared in analogy to example 2, step 2 by using 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 4-((tert-butoxycarbonyl)amino)butanoate instead of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-((tert-butoxycarbonyl)amino)butanoate was obtained as a white solid (0.22 g, yield: 99%). MS (m/z): [M+H]⁺ calcd for C₂₅H₃₅NO₆, 446.25; found, 446.2. ¹H NMR (400 MHz, Deuterium Oxide) δ 7.60 (d, J=10.0 Hz, 1H), 6.39 (dd, J=10.0, 1.9 Hz, 1H), 6.16 (s, 1H), 5.19 (d, J=18.0 Hz, 1H), 5.06 (d, J=18.0 Hz, 1H), 4.54 (d, J=3.5 Hz, 1H), 3.12 (q, J=6.3, 4.9 Hz, 2H), 2.72-2.55 (m, 4H), 2.46 (d, J=12.2 Hz, 1H), 2.21 (d, J=10.5 Hz, 1H), 2.10-1.96 (m, 3H), 1.83 (d, J=13.5 Hz, 2H), 1.73-1.52 (m, 3H), 1.46 (s, 3H), 1.38-1.29 (m, 1H), 1.17-1.12 (m, 2H), 0.90 (s, 3H).

### Step 3: Preparation of Conjugate of HA and 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 4-aminobutanoate

The title compound was prepared in analogy to example 34, step 3 by using 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 4-aminobutanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.204 g, yield: 67%, DSR=18%) ¹H NMR (400 MHz, D₂O) δ 7.61 (d, J=10.0 Hz, 0.18H), 6.39 (d, J=10.6 Hz, 0.18H), 6.17 (s, 0.18H), 5.20-4.96 (m, 0.36H), 4.69-4.35 (m, 2.18H), 4.06-2.97 (m, 10.36H), 2.76-2.41 (m, 0.9H), 2.25-1.82 (m, 4.26H), 1.73-1.40 (m, 1.08H), 1.26-1.10 (m, 0.36H), 0.91 (s, 0.54H).

With this procedure, reaction of sodium hyaluronate (MW 50 KDa) provided corresponding product (0.224 g, yield: 73.6%, DSR=23%). ¹H NMR (400 MHz, D₂O) δ 7.61 (d, J=10.1 Hz, 0.23H), 6.39 (d, J=10.1 Hz, 0.23H), 6.16 (s, 0.23H), 5.21-4.95 (m, 0.46H), 4.68-4.43 (m, 2.23H), 4.15-3.13 (m, 10.46H), 2.72-2.39 (m, 1.15H), 2.31-1.77 (m, 4.61H), 1.72-1.43 (m, 1.38H), 1.26-1.10 (m, 0.46H), 0.91 (s, 0.69H).

### Example 37

### Preparation of Conjugate of CS and 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 4-aminobutanoate

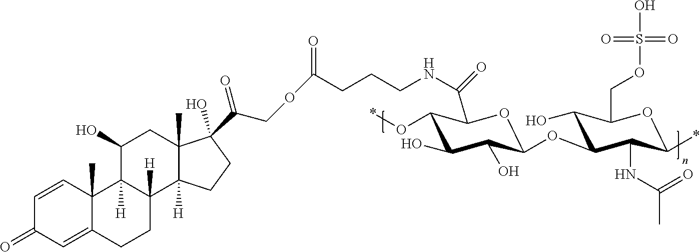

The title compound was prepared in analogy to example 36, step 3 by using CS (Chondroitin sulfate) instead of HA (Hyaluronic acid) was obtained as a white solid (0.232 g, yield: 68.5%, DSR=34%). ¹H NMR (400 MHz, D₂O) δ 7.68-7.57 (m, 0.34H), 6.41-6.34 (m, 0.34H), 6.16 (s, 0.34H), 5.24-4.95 (m, 0.68H), 4.68-4.43 (m, 2.34H), 4.27-3.18 (m, 10.68H), 2.70-2.41 (m, 1.7H), 2.24-1.82 (m, 5.38H), 1.71-1.41 (m, 2.04H), 1.22-1.15 (m, 0.68H), 0.91 (s, 1.02H).

### Example 38

### Preparation of Conjugate of HA and 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 4-aminobutanoate

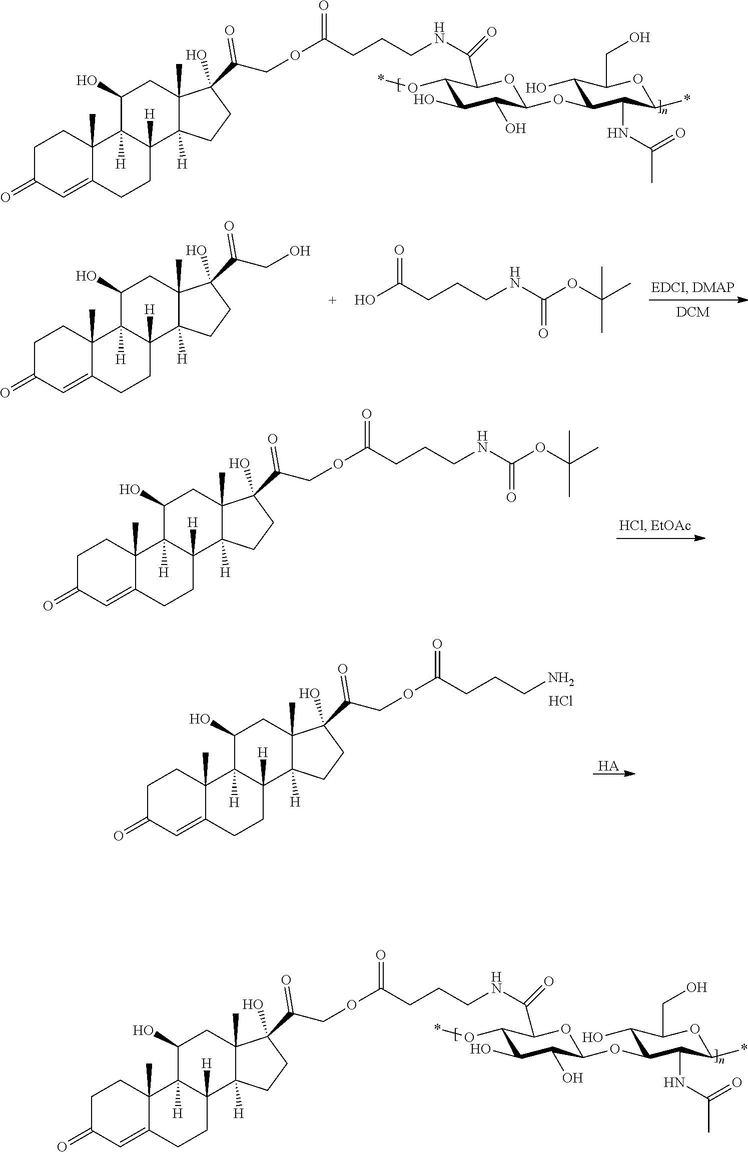

### Step 1: Preparation of 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 4-((tert-butoxycarbonyl)amino)butanoate

The title compound was prepared in analogy to example 2, step 1 by using (8S,9S,10R,11S,13S,14S,17R)-11,17-dihydroxy-17-(2-hydroxyacetyl)-10,13-dimethyl-1,2,6,7,8,9,10,11,12,13,14,15,16,17-tetradecahydro-3H-cyclopenta[a]phenanthren-3-one instead of 9α-fluoro-11β,16α,17,21-tetrahydroxy-1,4-pregnadiene-3,20-dione was obtained as a white solid (0.327 g, yield: 59.7%). MS (m/z): [M+H]⁺ calcd for C₃₀H₄₅NO₈, 548.31; found, 570.2 [M+Na]⁺.

### Step 2: Preparation of 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 4-aminobutanoate hydrochloride

The title compound was prepared in analogy to example 2, step 2 by using 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 4-((tert-butoxycarbonyl)amino)butanoate instead of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-((tert-butoxycarbonyl)amino)butanoate was obtained as a white solid (0.28 g, yield: 97%). MS (m/z): [M+H]⁺ calcd for C₂₅H₃₇NO₆, 448.26; found, 448.2. ¹H NMR (400 MHz, D₂O) δ 5.81 (s, 1H), 5.20 (d, J=18.0 Hz, 1H), 5.07 (d, J=18.0 Hz, 1H), 4.53 (d, J=2.8 Hz, 1H), 3.19-3.08 (m, 2H), 2.78-2.53 (m, 5H), 2.47-2.30 (m, 2H), 2.22 (dt, J=13.2, 4.6 Hz, 1H), 2.19-1.81 (m, 8H), 1.80-1.70 (m, 1H), 1.62 (ddd, J=13.9, 8.5, 5.5 Hz, 1H), 1.56-1.37 (m, 4H), 1.21-1.10 (m, 2H), 0.88 (s, 3H).

### Step 3: Preparation of Conjugate of HA and 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 4-aminobutanoate

The title compound was prepared in analogy to example 34, step 3 by using 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 4-aminobutanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.237 g, yield: 77.7%, DSR=28%). ¹H NMR (400 MHz, D₂O) δ 5.79 (s, 0.28H), 5.26-4.95 (m, 0.56H), 4.71-4.33 (m, 2.28H), 4.12-3.06 (m, 10H), 2.95-2.83 (m, 0.56H), 2.70-2.28 (m, 1.96H), 2.26-1.56 (m, 6.08H), 1.54-1.35 (m, 1.12H), 1.23-1.09 (m, 0.56H), 0.87 (s, 0.84H).

With this procedure, reaction of sodium hyaluronate (MW 50 KDa) provided corresponding product (0.196 g, yield: 64.3%, DSR=11%). ¹H NMR (400 MHz, D₂O) δ 5.79 (s, 0.11H), 5.11 (dd, J=51.2, 17.7 Hz, 0.22H), 4.66-4.24 (m, 2.11H), 4.17-3.02 (m, 10H), 2.97-2.80 (m, 0.22H), 2.72-2.27 (m, 0.77H), 2.26-1.56 (m, 4.21H), 1.54-1.37 (m, 0.44H), 1.23-1.11 (m, 0.22H), 0.87 (s, 0.33H).

### Example 39

### Preparation of Conjugate of CS and 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 4-aminobutanoate

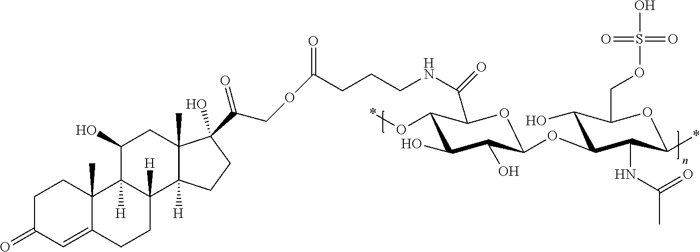

The title compound was prepared in analogy to example 38, step 3 by using CS (Chondroitin sulfate) instead of HA (Hyaluronic acid) was obtained as a white solid (0.275 g, yield: 81.1%, DSR=30%). ¹H NMR (400 MHz, D₂O) δ 5.80 (s, 0.3H), 5.13 (dd, J=47.6, 17.8 Hz, 0.6H), 4.68-4.38 (m, 2.3H), 4.30-3.15 (m, 10H), 2.97-2.88 (m, 0.6H), 2.68-2.29 (m, 2.1H), 2.28-1.58 (m, 6.3H), 1.52-1.33 (m, 1.2H), 1.29-1.06 (m, 0.6H), 0.87 (s, 0.9H).

### Example 40

### Preparation of Conjugate of HA and 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 4-aminobutanoate

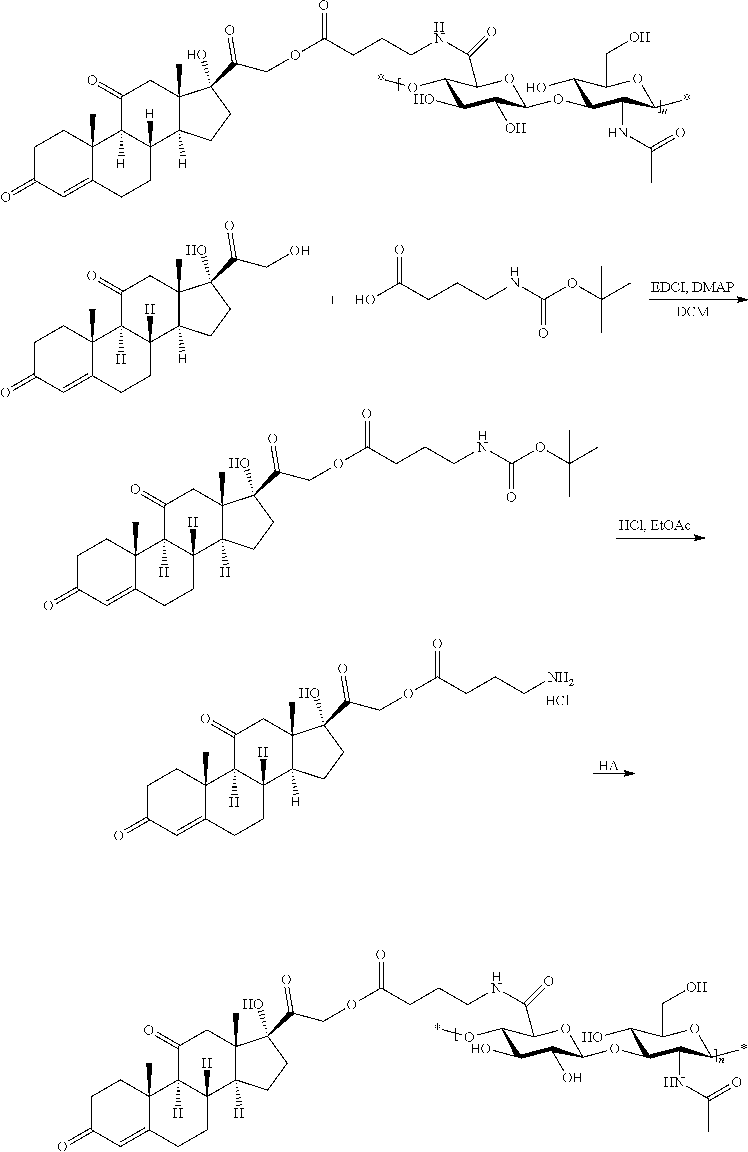

### Step 1: Preparation of 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 4-((tert-butoxycarbonyl)amino)butanoate

The title compound was prepared in analogy to example 2, step 1 by using (8S,9S,10R,13S,14S,17R)-17-hydroxy-17-(2-hydroxyacetyl)-10,13-dimethyl-1,6,7,8,9,10,12,13,14,15,16,17-dodecahydro-3H-cyclopenta[a]phenanthrene-3,11(2H)-dione instead of 9α-fluoro-11β,16α,17,21-tetrahydroxy-1,4-pregnadiene-3,20-dione was obtained as a white solid (0.68 g, yield: 89.7%). MS (m/z): [M+H]⁺ calcd for C₃₀H₄₃NO₈, 546.30; found, 568.2 [M+Na]⁺.

### Step 2: Preparation of 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 4-aminobutanoate hydrochloride

The title compound was prepared in analogy to example 2, step 2 by using 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 4-((tert-butoxycarbonyl)amino)butanoate instead of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-((tert-butoxycarbonyl)amino)butanoate was obtained as a white solid (0.48 g, yield: 98%). MS (m/z): [M+H]⁺ calcd for C₂₅H₃₅NO₆, 446.25; found, 446.2. ¹H NMR (400 MHz, D₂O) δ 5.85 (s, 1H), 5.16 (d, J=18.1 Hz, 1H), 5.03 (d, J=18.1 Hz, 1H), 3.19-3.07 (m, 2H), 2.97 (d, J=12.2 Hz, 1H), 2.70-2.51 (m, 6H), 2.43-2.25 (m, 5H), 2.22-1.94 (m, 5H), 1.87-1.66 (m, 2H), 1.56 (ddd, J=18.5, 12.2, 6.1 Hz, 1H), 1.51-1.32 (m, 4H), 0.66 (s, 3H).

### Step 3: Preparation of Conjugate of HA and 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 4-aminobutanoate

The title compound was prepared in analogy to example 34, step 3 by using 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 4-aminobutanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.243 g, yield: 79.8%, DSR=12%). ¹H NMR (400 MHz, D₂O) δ 5.84 (s, 0.12H), 5.19-4.98 (m, 0.24H), 4.64-4.35 (m, 2H), 4.09-2.98 (m, 10.24H), 2.92-2.85 (m, 0.12H), 2.73-2.27 (m, 1.32H), 2.26-1.49 (m, 3.96H), 1.48-1.25 (m, 0.48H), 0.64 (s, 0.36H).

With this procedure, reaction of sodium hyaluronate (MW 50 KDa) provided corresponding product (0.262 g, yield: 86.1%, DSR=8%). ¹H NMR (400 MHz, D₂O) δ 5.85 (s, 0.08H), 5.18-4.95 (m, 0.16H), 4.66-4.29 (m, 2H), 4.23-3.13 (m, 10.16H), 3.00-2.93 (m, 0.08H), 2.74-2.28 (m, 0.88H), 2.27-1.46 (m, 3.64H), 1.45-1.25 (m, 0.32H), 0.65 (s, 0.24H).

### Example 41

### Preparation of Conjugate of CS and 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 4-aminobutanoate

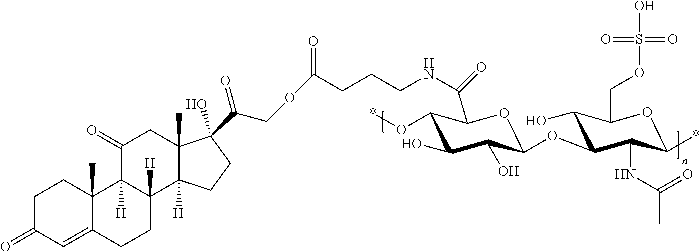

The title compound was prepared in analogy to example 40, step 3 by using CS (Chondroitin sulfate) instead of HA (Hyaluronic acid) was obtained as a white solid (0.235 g, yield: 69.4%, DSR=16%). ¹FH NMR (400 MHz, D₂O) δ 5.77 (s, 0.16H), 5.13-4.89 (m, 0.32H), 4.65-4.33 (m, 2H), 4.21-3.02 (m, 10.32H), 2.96-2.88 (m, 0.16H), 2.65-2.22 (m, 1.76H), 2.19-1.41 (m, 4.28H), 1.40-1.25 (m, 0.64H), 0.58 (s, 0.48H).

### Example 42

### Preparation of Conjugate of HA and 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 4-aminobutanoate

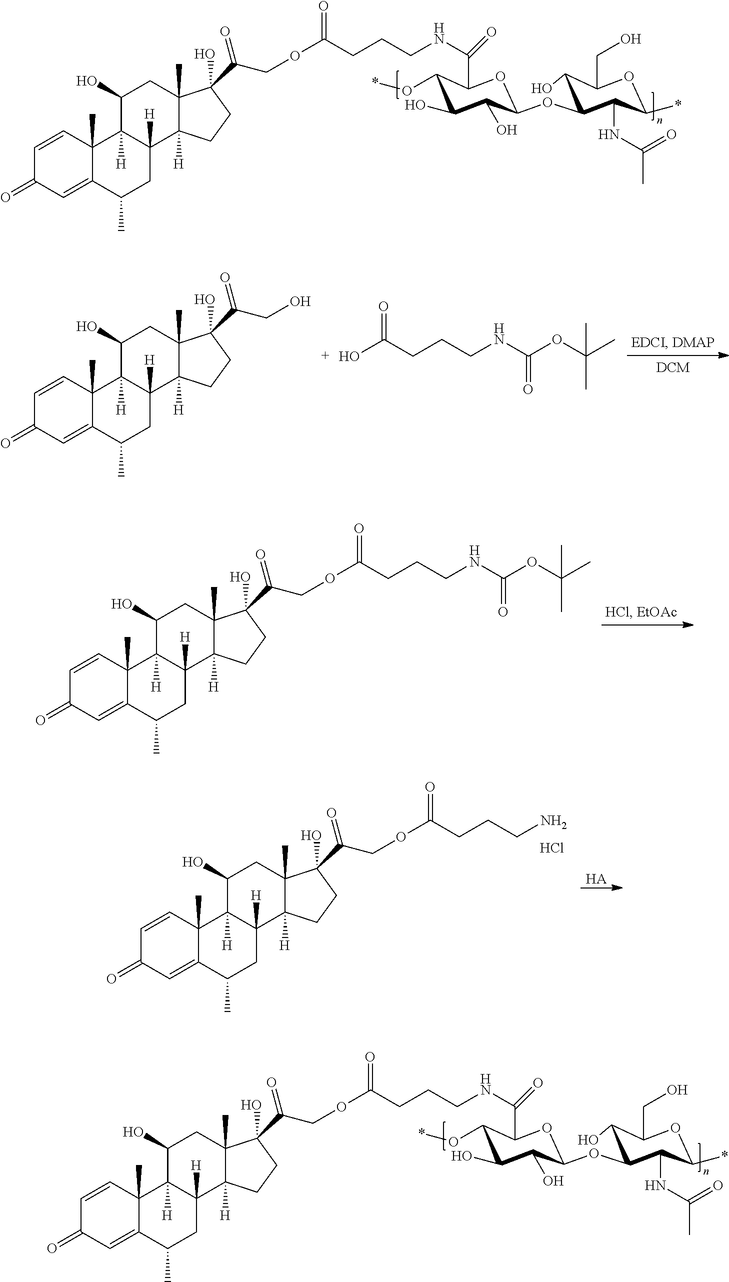

### Step 1: Preparation of 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 4-((tert-butoxycarbonyl)amino)butanoate

The title compound was prepared in analogy to example 2, step 1 by using (6S,8S,9S,10R,11S,13S,14S,17R)-11,17-dihydroxy-17-(2-hydroxyacetyl)-6,10,13-trimethyl-6,7,8,9,10,11,12,13,14,15,16,17-dodecahydro-3H-cyclopenta[a]phenanthren-3-one instead of 9α-fluoro-11β,16α,17,21-tetrahydroxy-1,4-pregnadiene-3,20-dione was obtained as a white solid (0.612 g, yield: 81.9%). MS (m/z): [M+H]⁺ calcd for C₃₁H₄₅NO₈, 560.31; found, 582.2 [M+Na]⁺.

### Step 2: Preparation of 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 4-aminobutanoate hydrochloride

The title compound was prepared in analogy to example 2, step 2 by using 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 4-((tert-butoxycarbonyl)amino)butanoate instead of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-((tert-butoxycarbonyl)amino)butanoate was obtained as a white solid (0.52 g, yield: 97.8%). MS (m/z): [M+H]⁺ calcd for C₂₆H₃₇NO₆, 460.26; found, 460.2. ¹H NMR (400 MHz, DMSO) δ 7.57 (d, J=10.0 Hz, 1H), 6.37 (d, J=10.1 Hz, 1H), 6.14-6.01 (m, 1H), 5.24-5.11 (m, 1H), 5.03 (d, J=17.9 Hz, 1H), 4.54-4.38 (m, 1H), 3.16-3.04 (m, 2H), 2.78-2.54 (m, 4H), 2.28-1.92 (m, 5H), 1.86-1.38 (m, 8H), 1.13 (d, J=5.6 Hz, 3H), 1.02-0.71 (m, 5H).

### Step 3: Preparation of Conjugate of HA and 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 4-aminobutanoate

The title compound was prepared in analogy to example 34, step 3 by using 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 4-aminobutanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.28 g, yield: 91%, DSR=22%). ¹H NMR (400 MHz, D₂O) δ 7.67-7.55 (m, 0.22H), 6.46-6.32 (m, 0.22H), 6.14-6.08 (m, 0.22H), 5.21-5.00 (m, 0.44H), 4.65-4.24 (m, 2.22H), 4.10-3.05 (m, 10.44H), 2.85-2.44 (m, 0.88H), 2.34-1.75 (m, 4.54H), 1.71-1.39 (m, 1.32H), 1.21-1.09 (m, 0.66H), 0.98-0.81 (m, 1.1H).

With this procedure, reaction of sodium hyaluronate (MW 50 KDa) provided corresponding product (0.277 g, yield: 90%, DSR=26%). ¹H NMR (400 MHz, D₂O) δ 7.67-7.56 (m, 0.26H), 6.44-6.35 (m, 0.26H), 6.15-6.08 (m, 0.26H), 5.17-4.97 (m, 0.52H), 4.65-4.40 (m, 2.26H), 4.16-3.12 (m, 10.52H), 2.86-2.39 (m, 1.04H), 2.35-1.72 (m, 4.82H), 1.71-1.38 (m, 1.56H), 1.20-1.08 (m, 0.78H), 0.98-0.81 (s, 1.3H).

### Example 43

### Preparation of Conjugate of CS and 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 4-aminobutanoate

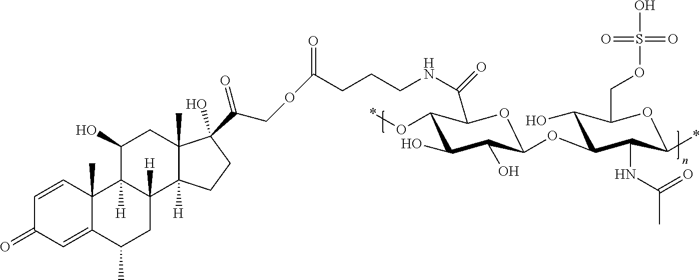

The title compound was prepared in analogy to example 42, step 3 by using CS (Chondroitin sulfate) instead of HA (Hyaluronic acid) was obtained as a white solid (0.27 g, yield: 79%, DSR=32%). ¹H NMR (400 MHz, D₂O) δ 7.66-7.54 (m, 0.32H), 6.45-6.34 (m, 0.32H), 6.15-6.06 (m, 0.32H), 5.22-4.96 (m, 0.64H), 4.67-4.41 (m, 2.32H), 4.32-3.13 (m, 10.64H), 2.83-2.41 (m, 1.28H), 2.33-1.70 (m, 5.24H), 1.68-1.32 (m, 1.92H), 1.19-1.06 (m, 0.96H), 0.98-0.80 (m, 1.6H).

### Example 44

### Preparation of Conjugate of HA and 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-aminobutanoate

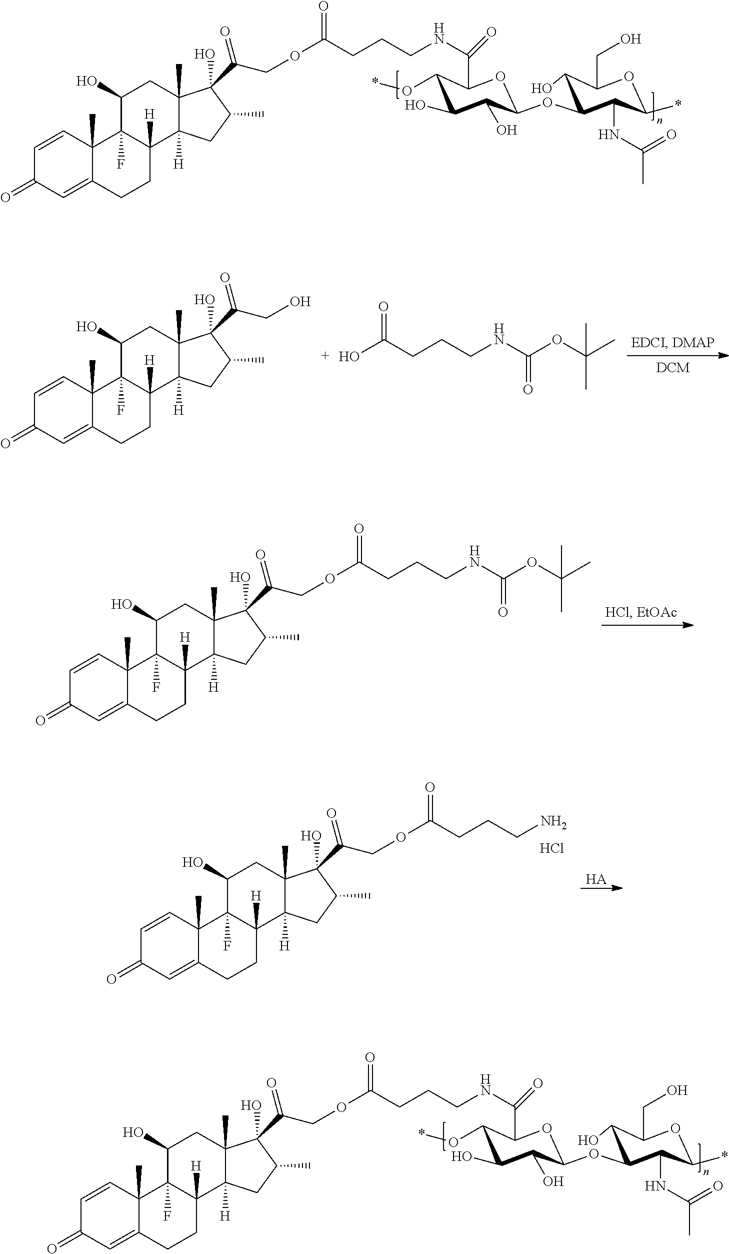

### Step 1: Preparation of 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-((tert-butoxycarbonyl)amino)butanoate

The title compound was prepared in analogy to example 2, step 1 by using (8S,9R,10S,11S,13S,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one instead of 9α-fluoro-11β,16α,17,21-tetrahydroxy-1,4-pregnadiene-3,20-dione was obtained as a white solid (0.61 g, yield: 82.8%). MS (m/z): [M+H]⁺ calcd for C₃₁H₄₄FNO₈, 578.31; found, 600.2 [M+Na]⁺.

### Step 2: Preparation of 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-aminobutanoate hydrochloride

The title compound was prepared in analogy to example 2, step 2 by using 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-((tert-butoxycarbonyl)amino)butanoate instead of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-((tert-butoxycarbonyl)amino)butanoate was obtained as a white solid (0.52 g, yield: 98.9%). MS (m/z): [M+H]⁺ calcd for C₂₆H₃₆FNO₆, 478.25; found, 478.2. ¹H NMR (400 MHz, D₂O) δ 7.55 (d, J=10.1 Hz, 1H), 6.45 (dd, J=10.2, 2.0 Hz, 1H), 6.26 (s, 1H), 5.10 (s, 2H), 4.48-4.30 (m, 1H), 3.12 (t, J=7.7 Hz, 2H), 2.90-2.43 (m, 5H), 2.29-1.91 (m, 7H), 1.58 (s, 4H), 1.34-1.17 (m, 2H), 1.12 (d, J=7.3 Hz, 3H), 1.02 (s, 3H).

### Step 3: Preparation of Conjugate of HA and 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-aminobutanoate

The title compound was prepared in analogy to example 34, step 3 by using 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-aminobutanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.251 g, yield: 80.5%, DSR=22%). ¹H NMR (400 MHz, D₂O) δ 7.56 (d, J=9.8 Hz, 0.22H), 6.45 (d, J=9.8 Hz, 0.22H), 6.25 (s, 0.22H), 5.08 (s, 0.44H), 4.66-4.29 (m, 2.22H), 4.17-3.03 (m, 10.44H), 2.95-2.35 (m, 1.1H), 2.31-1.73 (m, 4.54H), 1.72-1.44 (m, 0.88H), 1.33-1.27 (m, 0.44H), 1.11 (d, J=7.0 Hz, 0.66H), 1.03 (s, 0.66H).

With this procedure, reaction of sodium hyaluronate (MW 50 KDa) provided corresponding product (0.262 g, yield: 84.1%, DSR=15%). ¹H NMR (400 MHz, D₂O) δ 7.57 (d, J=9.9 Hz, 0.15H), 6.45 (d, J=10.0 Hz, 0.15H), 6.25 (s, 0.15H), 5.13 (s, 0.3H), 4.65-4.21 (m, 2.15H), 4.19-3.06 (m, 10.3H), 3.01-2.43 (m, 0.75H), 2.37-1.74 (m, 4.05H), 1.72-1.44 (m, 0.6H), 1.27-1.19 (m, 0.3H), 1.12 (d, J=7.1 Hz, 0.45H), 1.03 (s, 0.45H).

### Example 45

### Preparation of Conjugate of CS and 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-aminobutanoate

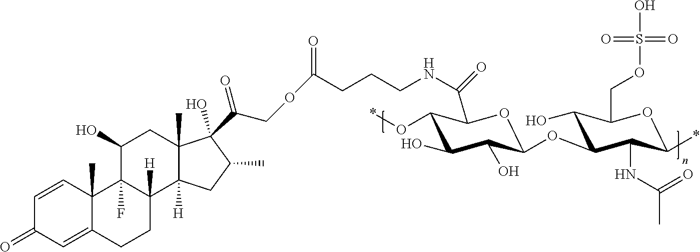

The title compound was prepared in analogy to Example 44, step 3 by using CS (Chondroitin sulfate) instead of HA (Hyaluronic acid) was obtained as a white solid (0.223 g, yield: 64.5%, DSR=25%). ¹FH NMR (400 MHz, D₂O) δ 7.57 (d, J=9.8 Hz, 0.25H), 6.45 (d, J=9.9 Hz, 0.25H), 6.25 (s, 0.25H), 5.08 (s, 0.5H), 4.68-4.35 (m, 2.25H), 4.32-3.16 (m, 10.5H), 3.00-2.42 (m, 1.25H), 2.38-1.80 (m, 4.75H), 1.74-1.47 (m, 1H), 1.32-1.17 (m, 0.5H), 1.12 (d, J=6.5 Hz, 0.75H), 1.02 (s, 0.75H).

### Example 46

### Preparation of Conjugate of HA and 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-aminobutanoate

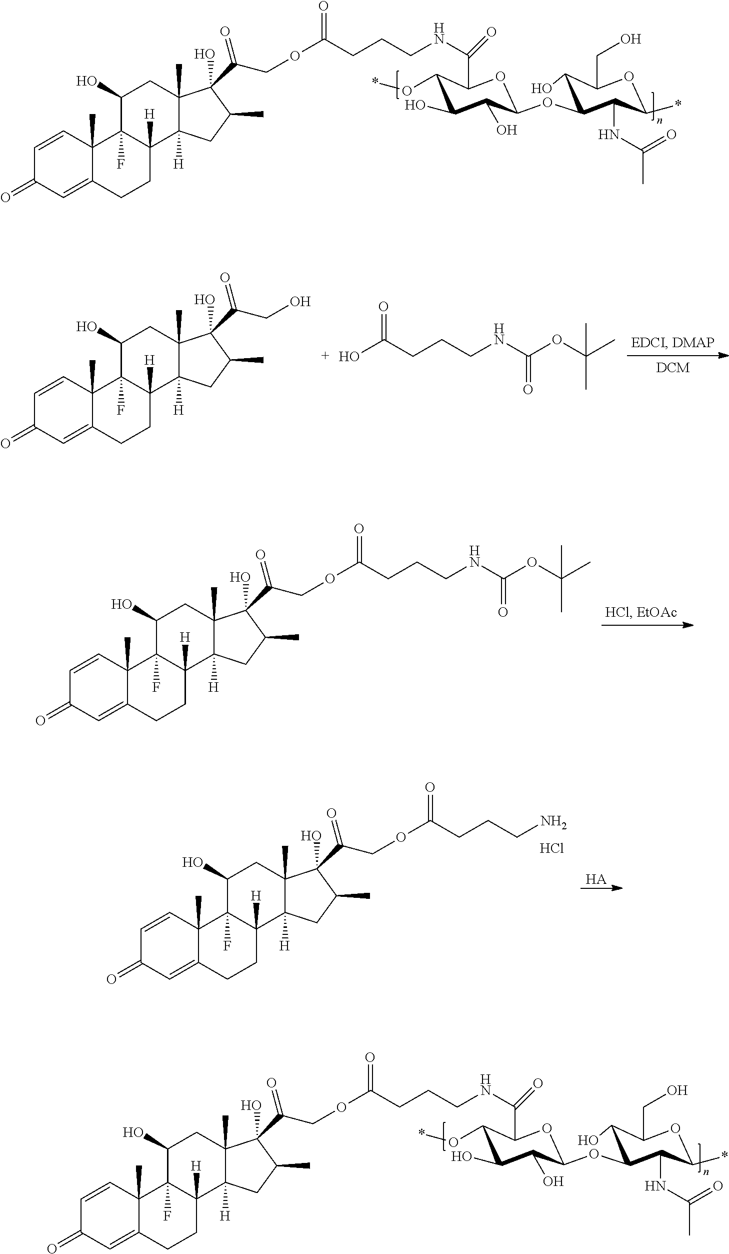

### Step 1: Preparation of 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-((tert-butoxycarbonyl)amino)butanoate

The title compound was prepared in analogy to example 2, step 1 by using (8S,9R,10S,11S,13S,14S,16S,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one instead of 9α-fluoro-11β,16α,17,21-tetrahydroxy-1,4-pregnadiene-3,20-dione was obtained as a white solid (0.37 g, yield: 94.2%). MS (m/z): [M+H]⁺ calcd for C₃₁H₄₄FNO₈, 578.31; found, 600.2 [M+Na]⁺.

### Step 2: Preparation of 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-aminobutanoate hydrochloride

The title compound was prepared in analogy to example 2, step 2 by using 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-((tert-butoxycarbonyl)amino)butanoate instead of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 4-((tert-butoxycarbonyl)amino)butanoate was obtained as a white solid (0.29 g, yield: 95.7%). MS (m/z): [M+H]⁺ calcd for C₂₆H₃₆FNO₆, 478.25; found, 478.2. ¹H NMR (400 MHz, D₂O) δ 7.56 (d, J=10.1 Hz, 1H), 6.46 (dd, J=10.1, 1.4 Hz, 1H), 6.26 (s, 1H), 5.17-5.02 (m, 2H), 4.43 (d, J=9.6 Hz, 1H), 3.19-3.04 (m, 2H), 2.82-2.42 (m, 5H), 2.37-1.96 (m, 5H), 1.95-1.71 (m, 2H), 1.70-1.44 (m, 4H), 1.29 (d, J=6.6 Hz, 2H), 1.01 (s, 3H), 0.91 (d, J=7.2 Hz, 3H).

### Step 3: Preparation of Conjugate of HA and 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-aminobutanoate

The title compound was prepared in analogy to example 34, step 3 by using 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-aminobutanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.238 g, yield: 76.3%, DSR=22%). ¹H NMR (400 MHz, D₂O) δ 7.57 (d, J=9.8 Hz, 0.22H), 6.45 (d, J=10.1 Hz, 0.22H), 6.25 (s, 0.22H), 5.09 (dd, J=32.2, 17.9 Hz, 0.44H), 4.65-4.21 (m, 2.22H), 4.20-2.82 (m, 10.44H), 2.58 (m, 1.1H), 2.37-1.67 (m, 4.54H), 1.64-1.40 (m, 0.88H), 1.35-1.21 (m, 0.44H), 1.01 (s, 0.66H), 0.90 (d, J=7.0 Hz, 0.66H).

With this procedure, reaction of sodium hyaluronate (MW 50 KDa) provided corresponding product (0.224 g, yield: 71.9%, DSR=12%). ¹H NMR (400 MHz, D₂O) δ 7.57 (d, J=10.0 Hz, 0.12H), 6.45 (d, J=9.8 Hz, 0.12H), 6.25 (s, 0.12H), 5.08 (q, J=18.0 Hz, 0.24H), 4.68-4.25 (m, 2.12H), 4.22-2.86 (m, 10.24H), 2.85-2.39 (m, 0.6H), 2.38-1.68 (m, 3.84H), 1.65-1.41 (m, 0.48H), 1.37-1.24 (m, 0.24H), 1.01 (s, 0.36H), 0.90 (d, J=7.0 Hz, 0.36H).

### Example 47

### Preparation of Conjugate of CS and 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 4-aminobutanoate

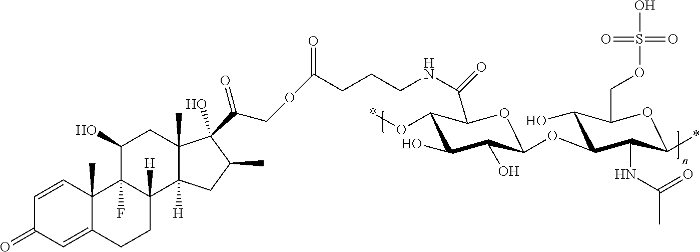

The title compound was prepared in analogy to example 46, step 3 by using CS (Chondroitin sulfate) instead of HA (Hyaluronic acid) was obtained as a white solid (0.243 g, yield: 70.3%, DSR=29%). ¹H NMR (400 MHz, D₂O) δ 7.58 (d, J=9.9 Hz, 0.29H), 6.45 (d, J=9.6 Hz, 0.29H), 6.25 (s, 0.29H), 5.09 (dd, J=33.2, 22.1 Hz, 0.58H), 4.68-4.34 (m, 2.29H), 4.32-2.84 (m, 10.58H), 2.83-2.36 (m, 1.45H), 2.35-1.66 (m, 5.03H), 1.65-1.38 (m, 1.16H), 1.34-1.12 (m, 0.58H), 1.01 (s, 0.87H), 0.90 (d, J=6.5 Hz, 0.87H).

### Example 48

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 7-azaspiro[3.5]nonane-2-carboxylate

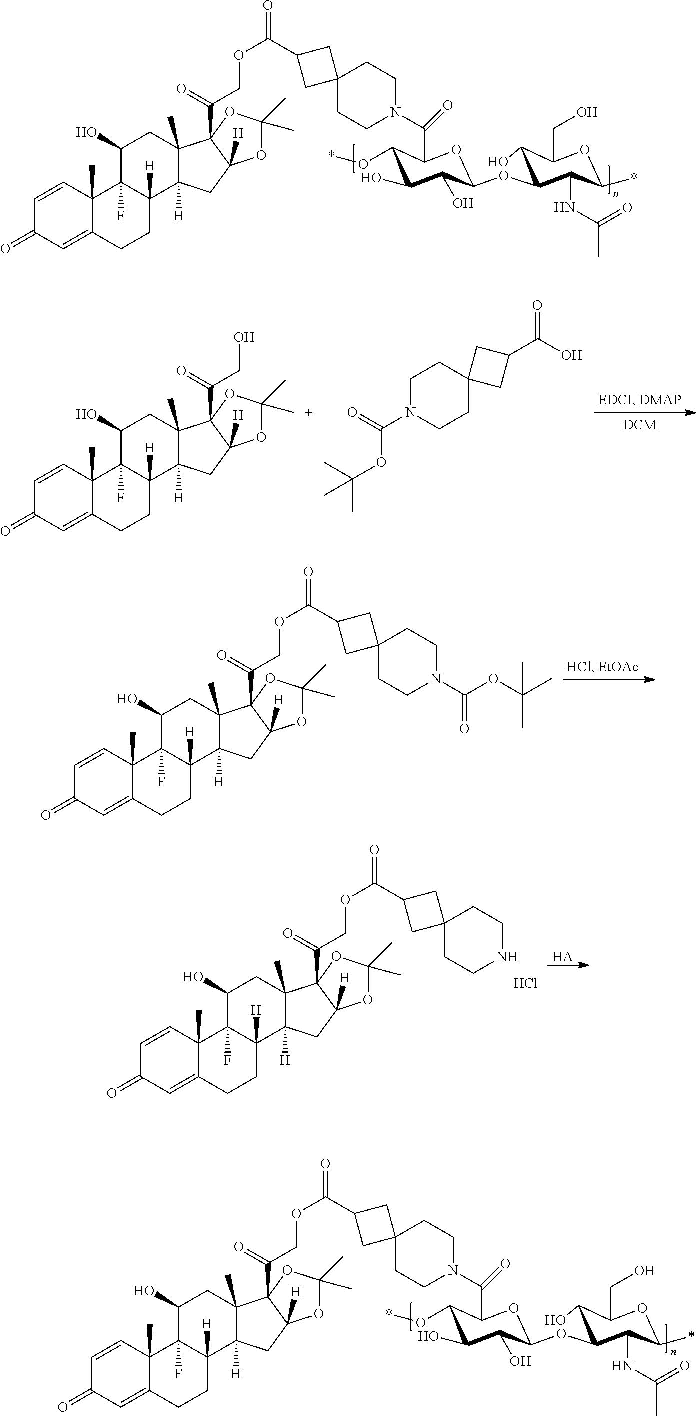

### Step 1: Preparation of 7-(tert-butyl) 2-(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) 7-azaspiro[3.5]nonane-2,7-dicarboxylate

The title compound was prepared in analogy to example 1, step 1 by using 7-(tert-butoxycarbonyl)-7-azaspiro[3.5]nonane-2-carboxylic acid instead of (tert-butoxycarbonyl)glycine was obtained as a white solid (0.525 g, yield: 97%). MS (m/z): [M+H]⁺ calcd for C₃₈H₅₂FNO₉, 686.36; found, 708.3 [M+Na]⁺. ¹H NMR (400 MHz, DMSO) δ 7.32 (d, J=10.1 Hz, 1H), 6.24 (dd, J=10.1, 1.8 Hz, 1H), 6.06-5.88 (m, 2H), 5.53 (d, J=4.0 Hz, 1H), 5.15 (d, J=17.9 Hz, 1H), 4.86 (d, J=4.7 Hz, 1H), 4.76 (d, J=17.9 Hz, 1H), 4.20 (s, 1H), 3.08-2.90 (m, 4H), 2.69-2.57 (m, 1H), 2.49-2.29 (m, 4.3 Hz, 2H), 2.16-1.79 (m, 7H), 1.77-1.64 (m, 3H), 1.63-1.47 (m, 5H), 1.46-1.28 (m, 15H), 1.19-1.08 (m, 3H), 0.90-0.75 (m, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 7-azaspiro[3.5]nonane-2-carboxylate hydrochloride

The title compound was prepared in analogy to example 1, step 1 by using 7-(tert-butyl) 2-(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) 7-azaspiro[3.5]nonane-2,7-dicarboxylate instead of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (tert-butoxycarbonyl)glycinate was obtained as a white solid (0.28 g, yield: 97%). MS (m/z): [M+H]⁺ calcd for C₃₃H₄₄FNO₇, 586.31; found, 586.3.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 7-azaspiro[3.5]nonane-2-carboxylate

The title compound was prepared in analogy to example 34, step 3 by using 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 7-azaspiro[3.5]nonane-2-carboxylate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.358 g, yield: 48.7%, DSR=9%). ¹H NMR (400 MHz, D₂O) δ 7.58-7.51 (m, 0.09H), 6.49-6.41 (m, 0.09H), 6.26 (s, 0.09H), 5.36-4.85 (m, 0.36H), 4.70-4.29 (m, 2.09H), 4.28-3.05 (m, 10.36H), 3.04-2.32 (m, 0.27H), 2.31-1.65 (m, 4.08H), 1.64-1.43 (m, 0.81H), 1.28 (s, 0.27H), 0.93 (s, 0.27H).

### Example 49

### Preparation of Conjugate of CS and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 7-azaspiro[3.5]nonane-2-carboxylate

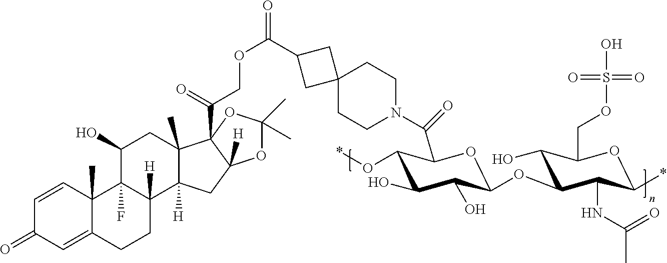

The title compound was prepared in analogy to example 48, step 3 by using CS (Chondroitin sulfate) instead of HA (Hyaluronic acid) was obtained as a white solid (0.18 g, yield: 62.9%, DSR=20%). ¹H NMR (400 MHz, D₂O) δ 7.63-7.50 (m, 0.2H), 6.47-6.40 (m, 0.2H), 6.33-6.23 (m, 0.2H), 5.21-4.87 (m, 0.8H), 4.69-4.39 (m, 2.2H), 4.38-3.02 (m, 10.8H), 3.00-2.30 (m, 0.6H), 2.29-1.67 (m, 5.4H), 1.66-1.44 (m, 1.8H), 1.29 (s, 0.6H), 0.94 (s, 0.6H).

### Example 50

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 14-amino-3,6,9,12-tetraoxatetradecanoate

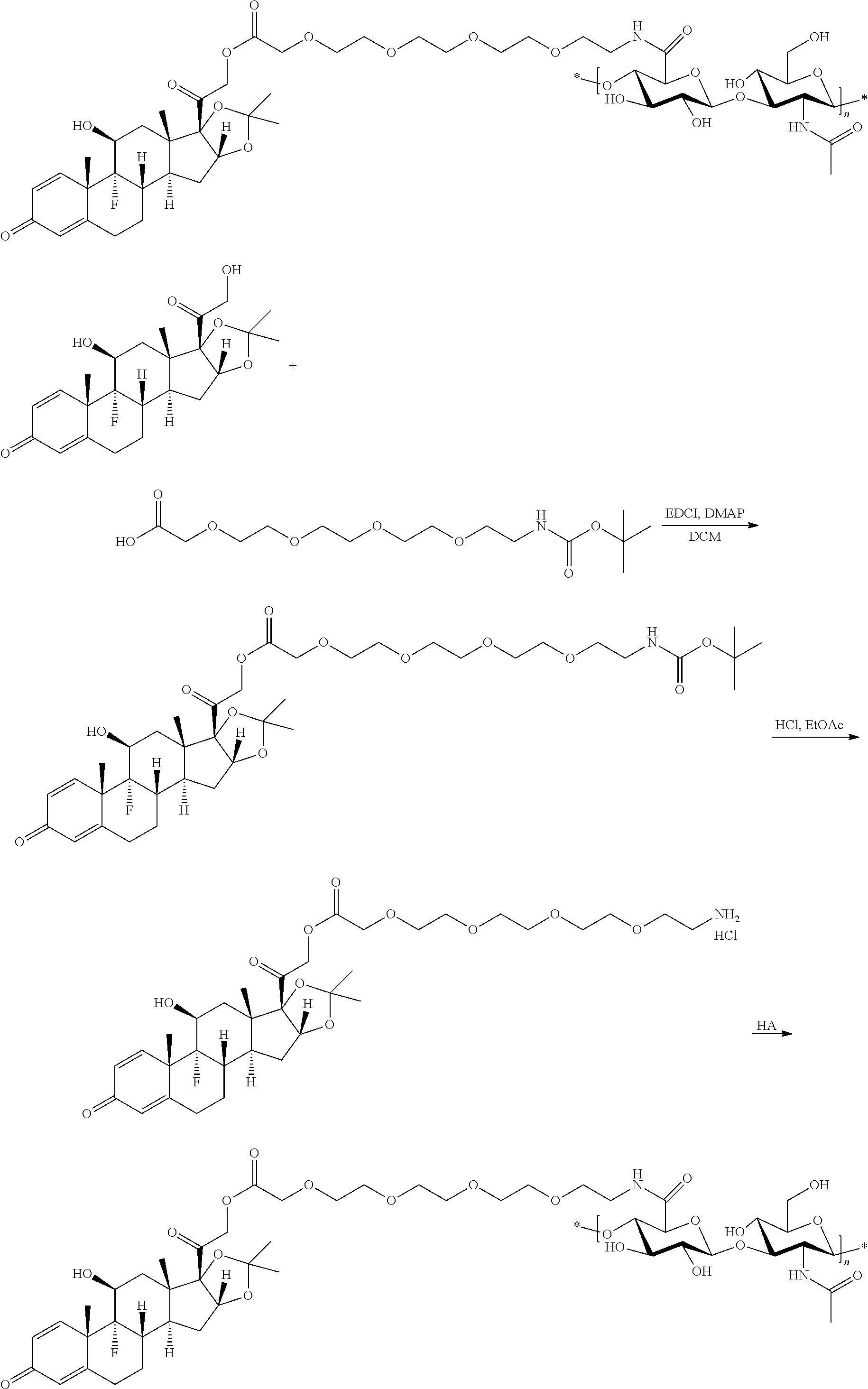

### Step 1: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2,2-dimethyl-4-oxo-3,8,11,14,17-pentaoxa-5-azanonadecan-19-oate

The title compound was prepared in analogy to example 1, step 1 by using 2,2-dimethyl-4-oxo-3,8,11,14,17-pentaoxa-5-azanonadecan-19-oic acid instead of (tert-butoxycarbonyl)glycine was obtained as a white solid (1.1 g, yield: 95.9%). MS (m/z): [M+H]⁺ calcd for C₃₉H₅₈FNO₁₃, 768.39; found, 790.3 [M+Na]⁺.

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 14-amino-3,6,9,12-tetraoxatetradecanoate hydrochloride

The title compound was prepared in analogy to example 1, step 1 by using 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2,2-dimethyl-4-oxo-3,8,11,14,17-pentaoxa-5-azanonadecan-19-oate instead of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl (tert-butoxycarbonyl)glycinate was obtained as a white solid (0.6 g, yield: 98.6%). MS (m/z): [M+H]⁺ calcd for C₃₄H₅₀FNO₁₁, 668.34; found, 668.3. ¹H NMR (400 MHz, DMSO) δ 7.91 (s, 4H), 7.34 (d, J=10.1 Hz, 1H), 6.24 (dd, J=10.1, 1.7 Hz, 1H), 6.02 (s, 1H), 5.58 (d, J=4.5 Hz, 1H), 5.23 (d, J=17.9 Hz, 1H), 5.00-4.73 (m, 2H), 4.35-4.20 (m, 3H), 3.73-3.58 (m, 14H), 3.09-2.92 (m, 2H), 2.64 (td, J=13.6, 5.8 Hz, 1H), 2.47-2.30 (m, 2H), 2.05 (d, J=13.5 Hz, 1H), 2.01-1.88 (m, 1H), 1.87-1.78 (m, 1H), 1.72 (d, J=13.2 Hz, 1H), 1.66-1.45 (m, 5H), 1.42-1.29 (m, 4H), 1.21-1.11 (m, 3H), 0.85 (d, J=8.4 Hz, 3H).

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 14-amino-3,6,9,12-tetraoxatetradecanoate

The title compound was prepared in analogy to example 34, step 3 by using 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 14-amino-3,6,9,12-tetraoxatetradecanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.235 g, yield: 93.6%, DSR=18%). ¹H NMR (400 MHz, D₂O) δ 7.62-7.52 (m, 0.18H), 6.53-6.43 (m, 0.18H), 6.27 (s, 0.18H), 5.42-5.27 (m, 0.18H), 5.19-4.90 (m, 0.36H), 4.72-4.31 (m, 2.54H), 4.30-3.05 (m, 12.88H), 3.04-2.28 (m, 0.54H), 2.27-1.65 (m, 3.72H), 1.64-1.44 (m, 1.62H), 1.26 (s, 0.54H), 0.93 (s, 0.54H).

### Example 51

### Preparation of Conjugate of CS and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 14-amino-3,6,9,12-tetraoxatetradecanoate

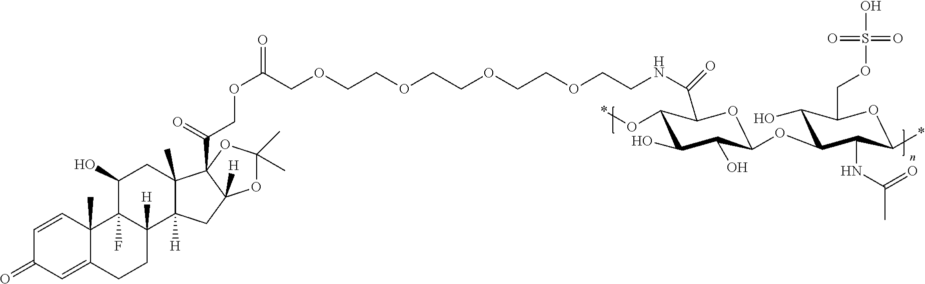

The title compound was prepared in analogy to example 50, step 3 by using CS (Chondroitin sulfate) instead of HA (Hyaluronic acid) was obtained as a white solid (0.268 g, yield: 97.8%, DSR=38%). ¹H NMR (400 MHz, D₂O) δ 7.62-7.52 (m, 0.38H), 6.53-6.42 (m, 0.38H), 6.26 (s, 0.38H), 5.41-5.28 (m, 0.38H), 5.20-4.91 (m, 0.76H), 4.75-4.37 (m, 3.14H), 4.35-3.00 (m, 16.08H), 2.98-2.28 (m, 1.14H), 2.27-1.67 (m, 4.52H), 1.66-1.44 (m, 3.42H), 1.26 (s, 1.14H), 0.92 (s, 1.14H).

### Example 52

### Preparation of Conjugate of HA and 2-aminopropyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

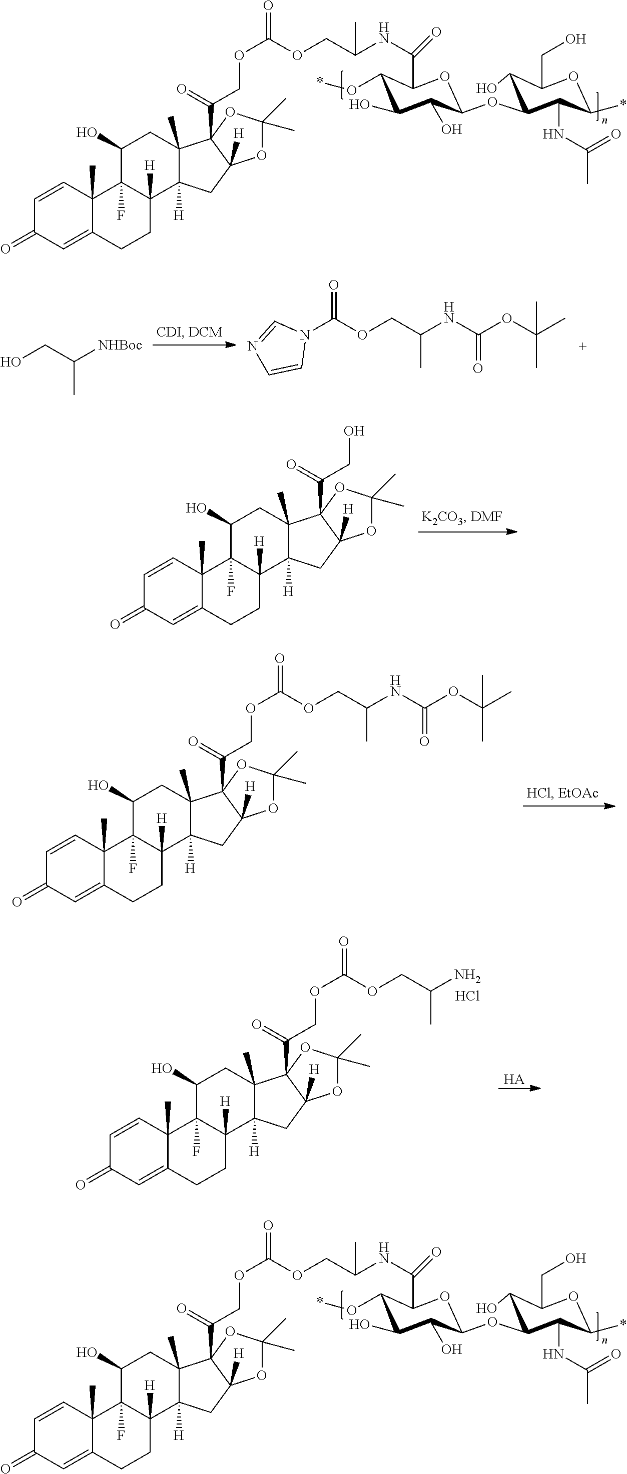

### Step 1: Preparation of 2-((tert-butoxycarbonyl)amino)propyl 1H-imidazole-1-carboxylate

The title compound was prepared in analogy to example 20, step 1 by using tert-butyl (1-hydroxypropan-2-yl)carbamate instead of tert-butyl (2-hydroxypropyl)carbamate was obtained as a white solid (0.716 g, yield: 88.7%). MS (m/z): [M+H]⁺ calcd for C₁₂H₁₉N₃O₄, 270.34; found, 270.2.

### Step 2: Preparation of tert-butyl (1-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,β1′:4,5]indeno[1,2-d][1,3]dioxol-8β-yl)-2-oxoethoxy)carbonyl)oxy)propan-2-yl)carbamate

The title compound was prepared in analogy to example 20, step 2 by using 2-((tert-butoxycarbonyl)amino)propyl 1H-imidazole-1-carboxylate instead of 1-((tert-butoxycarbonyl)amino)propan-2-yl 1H-imidazole-1-carboxylate was obtained as a white solid (0.58 g, yield: 39.7%). [M+H]⁺ calcd for C₃₃H₄₆FNO₁₀, 636.31; found, 658.3 [M+Na]⁺.

### Step 3: Preparation of 2-aminopropyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride

The title compound was prepared in analogy to example 20, step 3 by using tert-butyl (1-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,01′:4,5]indeno[1,2-d][1,3]dioxol-8β-yl)-2-oxoethoxy)carbonyl)oxy)propan-2-yl)carbamate instead of 1-aminopropan-2-yl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate was obtained as a white solid (0.5 g, yield: 98%). MS (m/z): [M+H]⁺ calcd for C₂₈H₃₈FNO₈, 536.26; found, 536.1. ¹H NMR (400 MHz, DMSO) δ 8.19 (s, 2H), 7.38 (d, J=10.1 Hz, 1H), 6.30 (dd, J=10.1, 1.4 Hz, 1H), 6.08 (s, 1H), 5.61 (s, 1H), 5.30 (dd, J=18.0, 3.4 Hz, 1H), 4.94 (d, J=4.5 Hz, 1H), 4.87 (dd, J=18.1, 2.3 Hz, 1H), 4.29 (dt, J=17.4, 9.6 Hz, 3H), 3.59 (d, J=4.6 Hz, 1H), 2.76-2.64 (m, 1H), 2.62-2.45 (m, 1H), 2.40 (d, J=9.6 Hz, 1H), 2.14-1.95 (m, 2H), 1.92-1.82 (m, 1H), 1.76 (d, J=13.1 Hz, 1H), 1.70-1.48 (m, 5H), 1.42 (s, 3H), 1.30 (t, J=8.8 Hz, 4H), 1.23-1.17 (m, 3H), 0.91 (d, J=7.7 Hz, 3H).

### Step 4: Preparation of Conjugate of HA and 2-aminopropyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

The title compound was prepared in analogy to example 34, step 3 by using 2-aminopropyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.206 g, yield: 67.1%, DSR=15%). ¹H NMR (400 MHz, Deuterium Oxide) δ 7.65-7.51 (m, 0.15H), 6.57-6.43 (m, 0.15H), 6.38-6.34 (m, 0.15H), 5.48-5.27 (m, 0.15H), 5.20-4.97 (m, 0.3H), 4.76-4.31 (m, 2.45H), 4.30-3.06 (m, 10.15H), 3.01-2.47 (m, 0.45H), 2.41-1.70 (m, 3.6H), 1.69-1.39 (m, 1.2H), 1.38-1.14 (m, 1.05H), 1.10-0.87 (m, 0.45H).

### Example 53

### Preparation of Conjugate of CS (Chondroitin sulfate) and 2-aminopropyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

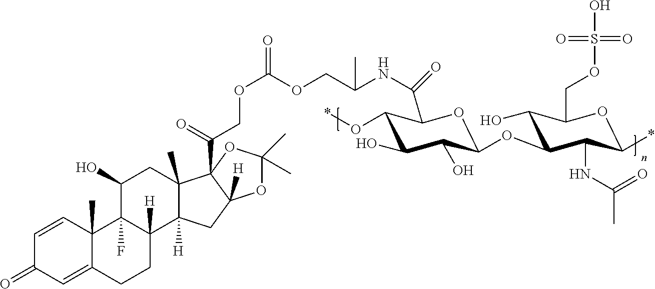

The title compound was prepared in analogy to example 52, step 4 by using CS (Chondroitin sulfate) instead of HA (Hyaluronic acid) was obtained as a white solid (0.188 g, yield: 61.4%, DSR=25%). ¹H NMR (400 MHz, Deuterium Oxide) δ 7.65-7.51 (m, 0.25H), 6.53-6.41 (m, 0.25H), 6.31-6.19 (m, 0.25H), 5.43-5.20 (m, 0.25H), 5.16-4.91 (m, 0.5H), 4.75-4.41 (m, 2.75H), 4.38-3.20 (m, 10.25H), 3.02-2.47 (m, 0.75H), 2.29-1.64 (m, 4H), 1.63-1.39 (m, 2H), 1.38-1.17 (m, 1.75H), 1.02-0.87 (m, 0.75H).

### Example 54

### Preparation of Conjugate of HA and 2-(2-(2-(2-aminoethoxy)ethoxy)ethoxy)ethyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

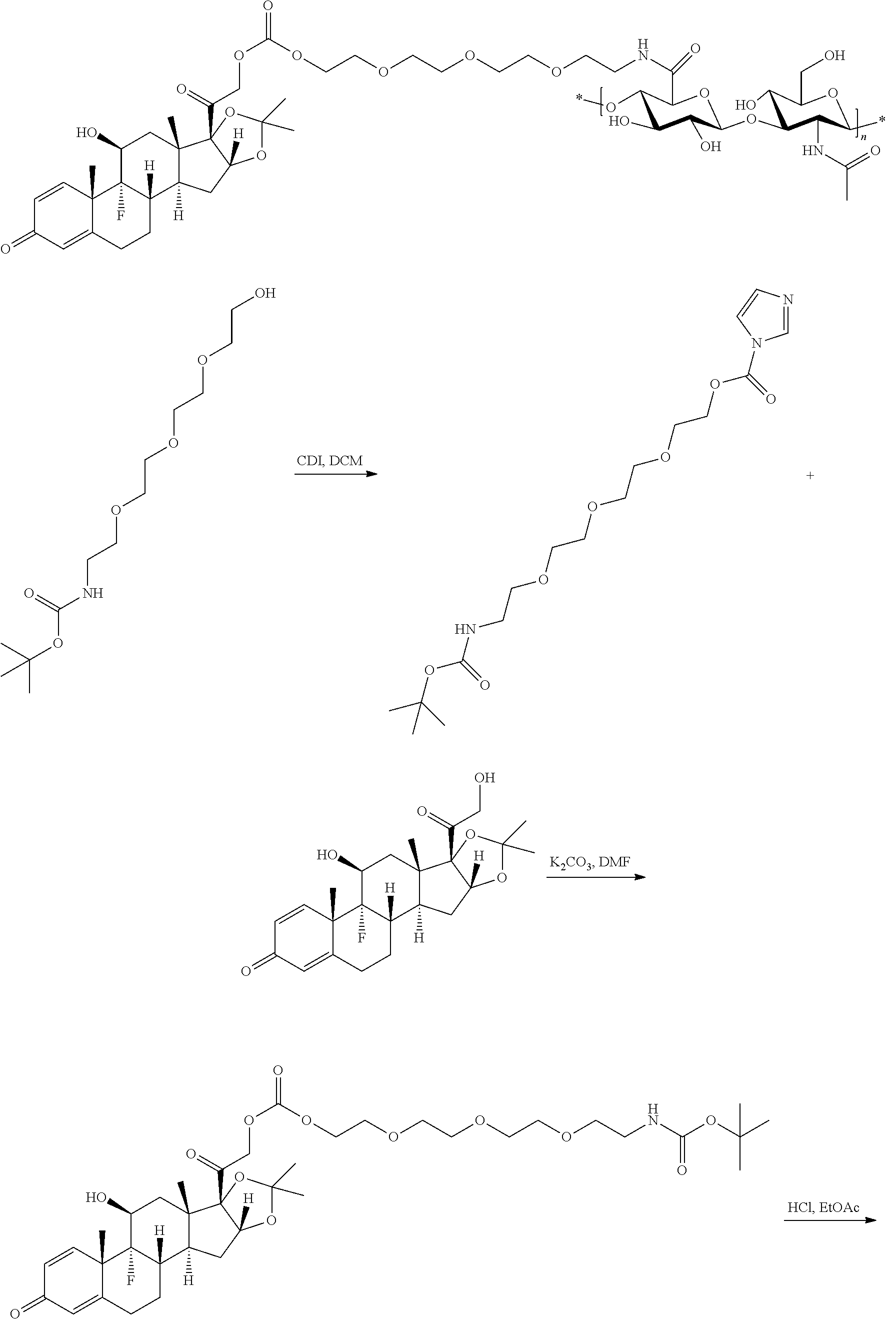

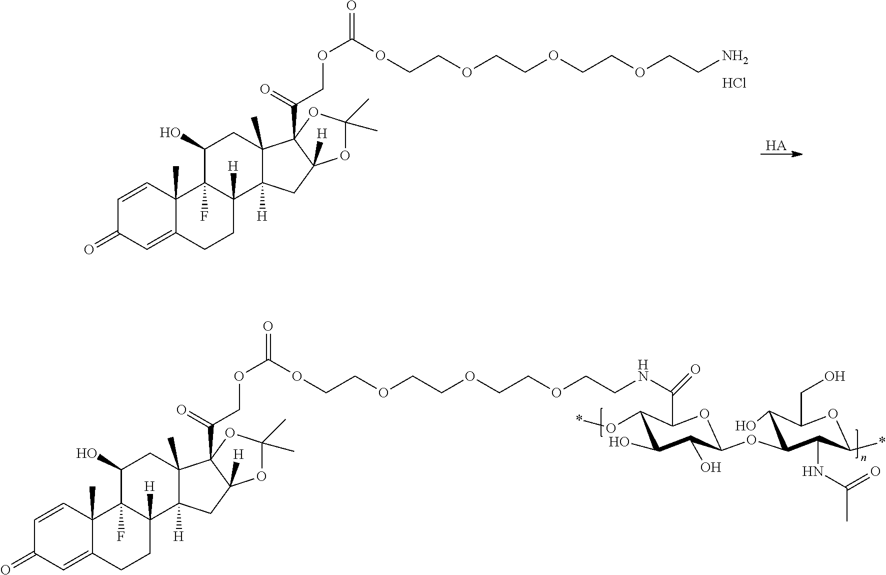

### Step 1: Preparation of 2,2-dimethyl-4-oxo-3,8,11,14-tetraoxa-5-azahexadecan-16-yl 1H-imidazole-1-carboxylate

The title compound was prepared in analogy to example 20, step 1 by using tert-butyl (2-(2-(2-(2-hydroxyethoxy)ethoxy)ethoxy)ethyl)carbamate instead of tert-butyl (2-hydroxypropyl)carbamate was obtained as a white solid (1 g, yield: 98%). MS (m/z): [M+H]⁺ calcd for C₁₇H₂₉N₃O₇, 388.20; found, 388.2.

### Step 2: Preparation of tert-butyl (1-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-1,4-dioxo-3,5,8,11,14-pentaoxahexadecan-16-yl)carbamate

The title compound was prepared in analogy to example 20, step 2 by using 2,2-dimethyl-4-oxo-3,8,11,14-tetraoxa-5-azahexadecan-16-yl 1H-imidazole-1-carboxylate instead of 1-((tert-butoxycarbonyl)amino)propan-2-yl 1H-imidazole-1-carboxylate was obtained as a white solid (0.48 g, yield: 92.3%). [M+H]⁺ calcd for C₃₈H₅₆FNO₁₃, 754.37; found, 776.3 [M+Na]⁺.

### Step 3: Preparation of 2-(2-(2-(2-aminoethoxy)ethoxy)ethoxy)ethyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate hydrochloride

The title compound was prepared in analogy to example 20, step 3 by using tert-butyl (1-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-1,4-dioxo-3,5,8,11,14-pentaoxahexadecan-16-yl)carbamate instead of 1-aminopropan-2-yl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate was obtained as a white solid (0.4 g, yield: 96%). MS (m/z): [M+H]⁺ calcd for C₃₃H₄₈FNO₁₁, 654.32; found, 654.2. ¹H NMR (400 MHz, DMSO) δ 7.90 (s, 4H), 7.32 (d, J=9.6 Hz, 1H), 6.23 (d, J=9.9 Hz, 1H), 6.01 (s, 1H), 5.56 (d, J=4.2 Hz, 1H), 5.18 (d, J=18.0 Hz, 1H), 4.87 (t, J=6.3 Hz, 1H), 4.75 (d, J=18.2 Hz, 1H), 4.30-4.07 (m, 3H), 3.76-3.45 (m, 12H), 2.92 (d, J=28.1 Hz, 2H), 2.62 (s, 1H), 2.30 (d, J=38.0 Hz, 2H), 2.16-1.86 (m, 2H), 1.73 (dd, J=33.1, 16.7 Hz, 1H), 1.64-1.43 (m, 3H), 1.42-1.20 (m, 7H), 1.14 (s, 3H), 0.87-0.72 (m, 3H).

### Step 4: Preparation of Conjugate of HA and 2-(2-(2-(2-aminoethoxy)ethoxy)ethoxy)ethyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

The title compound was prepared in analogy to example 34, step 3 by using 2-(2-(2-(2-aminoethoxy)ethoxy)ethoxy)ethyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.262 g, yield: 84.5%, DSR=7%). ¹H NMR (400 MHz, D₂O) δ 7.63-7.48 (m, 0.07H), 6.53-6.42 (m, 0.07H), 6.26 (s, 0.07H), 5.40-4.98 (m, 0.21H), 4.78-4.30 (m, 2.21H), 4.13-2.84 (m, 10.84H), 2.83-2.23 (m, 0.35H), 2.21-1.67 (m, 3.42H), 1.66-1.42 (m, 0.49H), 1.27 (s, 0.21H), 0.94 (s, 0.21H).

### Example 55

### Preparation of Conjugate of CS and 2-(2-(2-(2-aminoethoxy)ethoxy)ethoxy)ethyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) carbonate

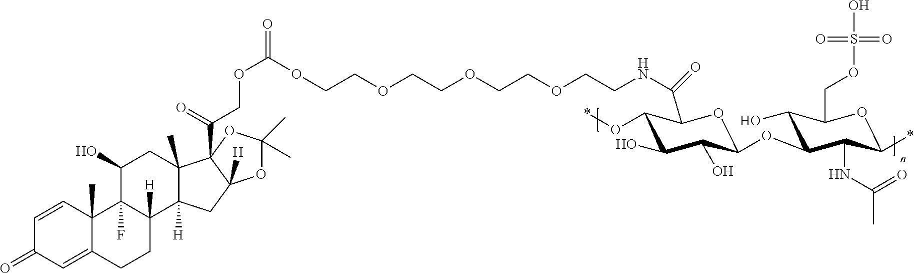

The title compound was prepared in analogy to example 54, step 3 by using CS (Chondroitin sulfate) instead of HA (Hyaluronic acid) was obtained as a white solid (0.252 g, yield: 77.3%, DSR=17%). ¹H NMR (400 MHz, D₂O) δ 7.64-7.49 (m, 0.17H), 6.54-6.42 (m, 0.17H), 6.27 (s, 0.17H), 5.40-4.98 (m, 0.51H), 4.77-4.33 (m, 2.51H), 4.31-3.02 (m, 12.04H), 3.01-2.23 (m, 0.85H), 2.21-1.67 (m, 4.02H), 1.66-1.42 (m, 1.19H), 1.29 (s, 0.51H), 0.95 (s, 0.51H).

### Example 56

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl piperazine-1-carboxylate

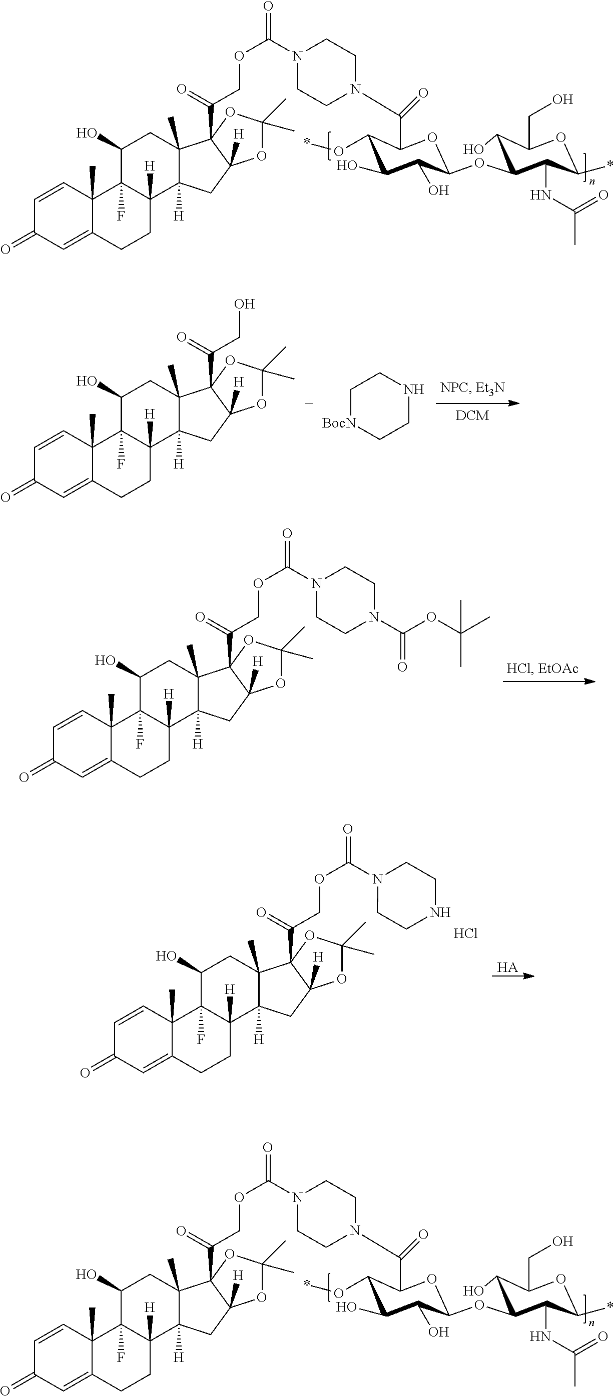

### Step 1: Preparation of 1-(tert-butyl) 4-(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) piperazine-1,4-dicarboxylate

The title compound was prepared in analogy to example 4, step 1 by using tert-butyl piperazine-1-carboxylate instead of tert-butyl (4-aminobutyl) carbamate was obtained as a white solid (0.44 g, yield: 98.6%). MS (m/z): [M+H]⁺ calcd for C₃₄H₄₇FN₂O₉, 647.33; found, 669.1 [M+Na]⁺. ¹H NMR (400 MHz, DMSO) δ 7.28 (d, J=10.1 Hz, 1H), 6.23 (dd, J=10.1, 1.7 Hz, 1H), 6.01 (s, 1H), 5.47 (d, J=4.2 Hz, 1H), 5.11 (d, J=18.1 Hz, 1H), 4.86 (t, J=9.4 Hz, 1H), 4.73 (d, J=18.1 Hz, 1H), 4.19 (s, 1H), 3.52-3.29 (m, 8H), 2.62 (td, J=13.3, 5.3 Hz, 1H), 2.48-2.27 (m, 2H), 2.03 (d, J=13.5 Hz, 1H), 1.98-1.88 (m, 1H), 1.87-1.76 (m, 1H), 1.71 (d, J=13.1 Hz, 1H), 1.61-1.45 (m, 5H), 1.44-1.20 (m, 13H), 1.16-1.08 (m, 3H), 0.83 (s, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl piperazine-1-carboxylate hydrochloride

The title compound was prepared in analogy to example 4, step 2 by using 1-(tert-butyl) 4-(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) piperazine-1,4-dicarboxylate instead of tert-butyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) butane-1,4-diyldicarbamate was obtained as a white solid (0.218 g, yield: 80%). MS (m/z): [M+H]⁺ calcd for C₂₉H₃₉FN₂O₇, 547.27; found, 547.2. ¹H NMR (400 MHz, DMSO) δ 9.31 (s, 3H), 7.38-7.27 (m, 1H), 6.24 (dd, J=10.1, 1.8 Hz, 1H), 6.02 (s, 1H), 5.58 (d, J=4.6 Hz, 1H), 5.15 (d, J=18.1 Hz, 1H), 4.87 (d, J=4.6 Hz, 1H), 4.76 (d, J=18.1 Hz, 1H), 4.20 (s, 1H), 3.65 (s, 4H), 3.11 (s, 4H), 2.70-2.57 (m, 1H), 2.49-2.39 (m, 2H), 2.34 (dd, J=13.6, 3.6 Hz, 1H), 2.10-1.89 (m, 2H), 1.87-1.77 (m, 1H), 1.71 (d, J=13.3 Hz, 1H), 1.64-1.44 (m, 5H), 1.43-1.27 (m, 4H), 1.24 (s, 3H), 0.82 (s, 3H).

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl piperazine-1-carboxylate

The title compound was prepared in analogy to example 34, step 3 by using 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl piperazine-1-carboxylate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.16 g, yield: 69.5%, DSR=30%). ¹H NMR (400 MHz, D₂O) δ 7.61-7.48 (m, 0.3H), 6.51-6.36 (m, 0.3H), 6.23 (s, 0.3H), 5.39-4.88 (m, 0.9H), 4.68-4.37 (m, 2.3H), 4.11-3.00 (m, 12.4H), 2.87-2.14 (m, 0.9H), 2.13-1.62 (m, 4.2H), 1.60-1.31 (m, 2.7H), 1.26 (s, 0.9H), 0.91 (s, 0.9H).

### Example 57

### Preparation of Conjugate of CS and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl piperazine-1-carboxylate

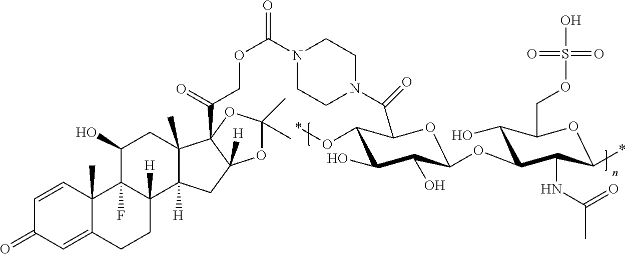

The title compound was prepared in analogy to example 56, step 3 by using CS (Chondroitin sulfate) instead of HA (Hyaluronic acid) was obtained as a white solid (0.15 g, yield: 60%, DSR=25%). ¹H NMR (400 MHz, D₂O) δ 7.61-7.49 (m, 0.25H), 6.50-6.42 (m, 0.25H), 6.26 (s, 0.25H), 5.38-4.95 (m, 0.75H), 4.73-4.38 (m, 2.25H), 4.35-3.11 (m, 12H), 3.01-2.21 (m, 0.75H), 2.17-1.67 (m, 1H), 1.66-1.42 (m, 2.25H), 1.29 (s, 0.75H), 0.94 (s, 0.75H).

### Example 58

### Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2,5-diazabicyclo[2.2.1]heptane-2-carboxylate

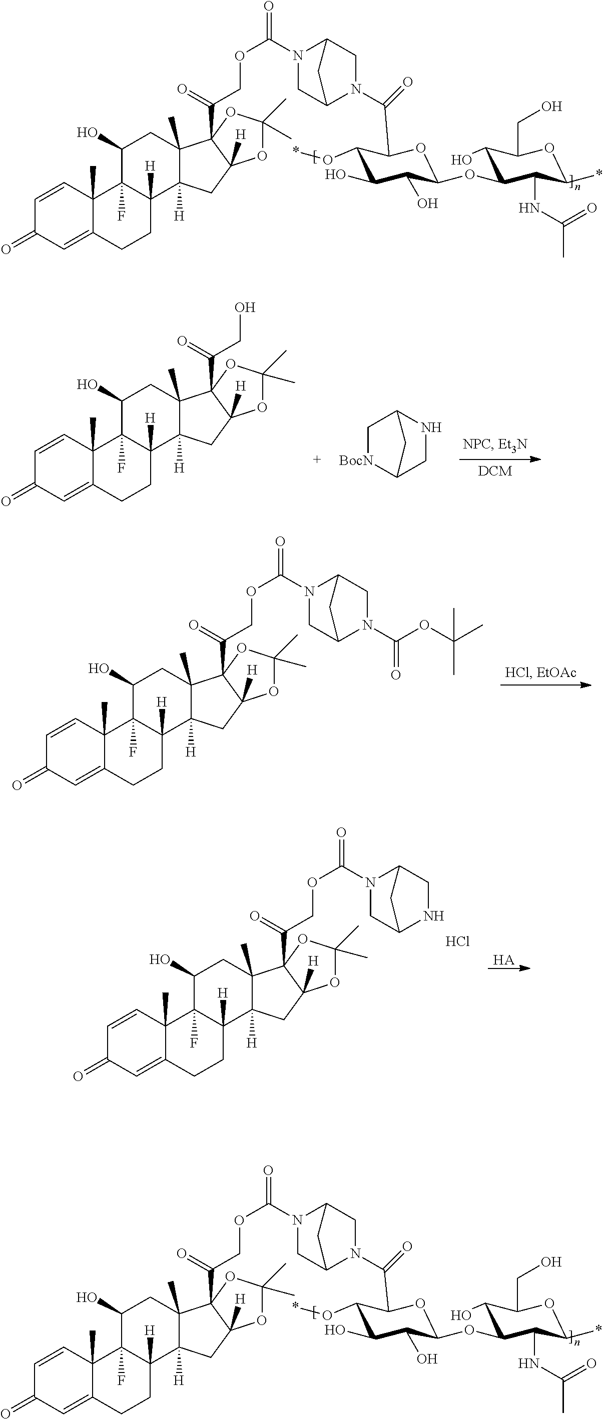

### Step 1: Preparation of 2-(tert-butyl) 5-(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) 2,5-diazabicyclo[2.2.1]heptane-2,5-dicarboxylate

The title compound was prepared in analogy to example 4, step 1 by using tert-butyl 2,5-diazabicyclo[2.2.1]heptane-2-carboxylate instead of tert-butyl (4-aminobutyl) carbamate was obtained as a white solid (0.442 g, yield: 97.3%). MS (m/z): [M+H]⁺ calcd for C₃₅H₄₇FN₂O₉, 659.33; found, 681.2 [M+Na]⁺. ¹H NMR (400 MHz, D₂O) δ 7.28 (d, J=10.1 Hz, 1H), 6.22 (dd, J=10.1, 1.8 Hz, 1H), 6.00 (s, 1H), 5.45 (s, 1H), 5.08 (dd, J=32.7, 18.4 Hz, 1H), 4.93-4.60 (m, 2H), 4.53-4.31 (m, 2H), 4.18 (s, 1H), 3.49-3.36 (m, 1H), 3.21 (d, J=10.6 Hz, 3H), 2.62 (td, J=13.3, 5.9 Hz, 1H), 2.48-2.28 (m, 2H), 2.08-1.89 (m, 2H), 1.84 (dd, J=17.7, 10.6 Hz, 3H), 1.71 (d, J=13.6 Hz, 1H), 1.62-1.46 (m, 5H), 1.40 (s, 8H), 1.37-1.29 (m, 4H), 1.22 (s, 1H), 1.14 (d, J=6.6 Hz, 3H), 0.88-0.74 (m, 3H).

### Step 2: Preparation of 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2,5-diazabicyclo[2.2.1]heptane-2-carboxylate hydrochloride

The title compound was prepared in analogy to example 4, step 2 by using 2-(tert-butyl) 5-(2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) 2,5-diazabicyclo[2.2.1]heptane-2,5-dicarboxylate instead of tert-butyl (2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl) butane-1,4-diyldicarbamate was obtained as a white solid (0.25 g, yield: 98%). MS (m/z): [M+H]⁺ calcd for C₃₀H₃₉FN₂O₇, 559.27; found, 559.1.

### Step 3: Preparation of Conjugate of HA and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2,5-diazabicyclo[2.2.1]heptane-2-carboxylate

The title compound was prepared in analogy to example 34, step 3 by using 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2,5-diazabicyclo[2.2.1]heptane-2-carboxylate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.203 g, yield: 70.9%, DSR=33%). ¹H NMR (400 MHz, D₂O) δ 7.57-7.46 (m, 0.33H), 6.45-6.36 (m, 0.33H), 6.22 (s, 0.33H), 5.33-4.92 (m, 0.99H), 4.71-4.30 (m, 2.99H), 4.11-2.97 (m, 11.32H), 2.91-2.10 (m, 0.99H), 2.09-1.61 (m, 4.98H), 1.60-1.38 (m, 2.97H), 1.24 (s, 0.99H), 0.89 (s, 0.99H).

### Example 59

### Preparation of Conjugate of CS and 2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 2,5-diazabicyclo[2.2.1]heptane-2-carboxylate

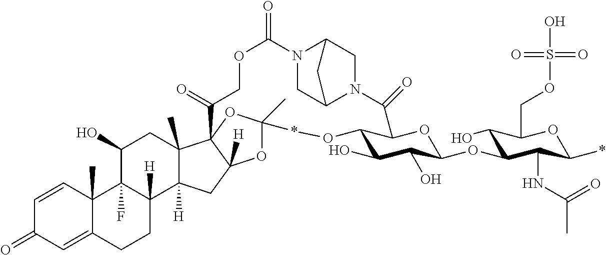

The title compound was prepared in analogy to example 58, step 3 by using CS (Chondroitin sulfate) instead of HA (Hyaluronic acid) was obtained as a white solid (0.236 g, yield: 75.3%, DSR=26%). ¹FH NMR (400 MHz, D₂O) δ 7.62-7.47 (m, 0.26H), 6.51-6.36 (m, 0.26H), 6.26 (s, 0.26H), 5.36-4.97 (m, 0.78H), 4.73-4.38 (m, 2.78H), 4.33-3.11 (m, 11.04H), 2.94-2.18 (m, 0.78H), 2.17-1.65 (m, 4.56H), 1.64-1.39 (m, 2.34H), 1.28 (s, 0.78H), 0.94 (s, 0.78H).

### Example 60

### Preparation of Conjugate of HA and 4-aminobutyl (2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl) carbonate

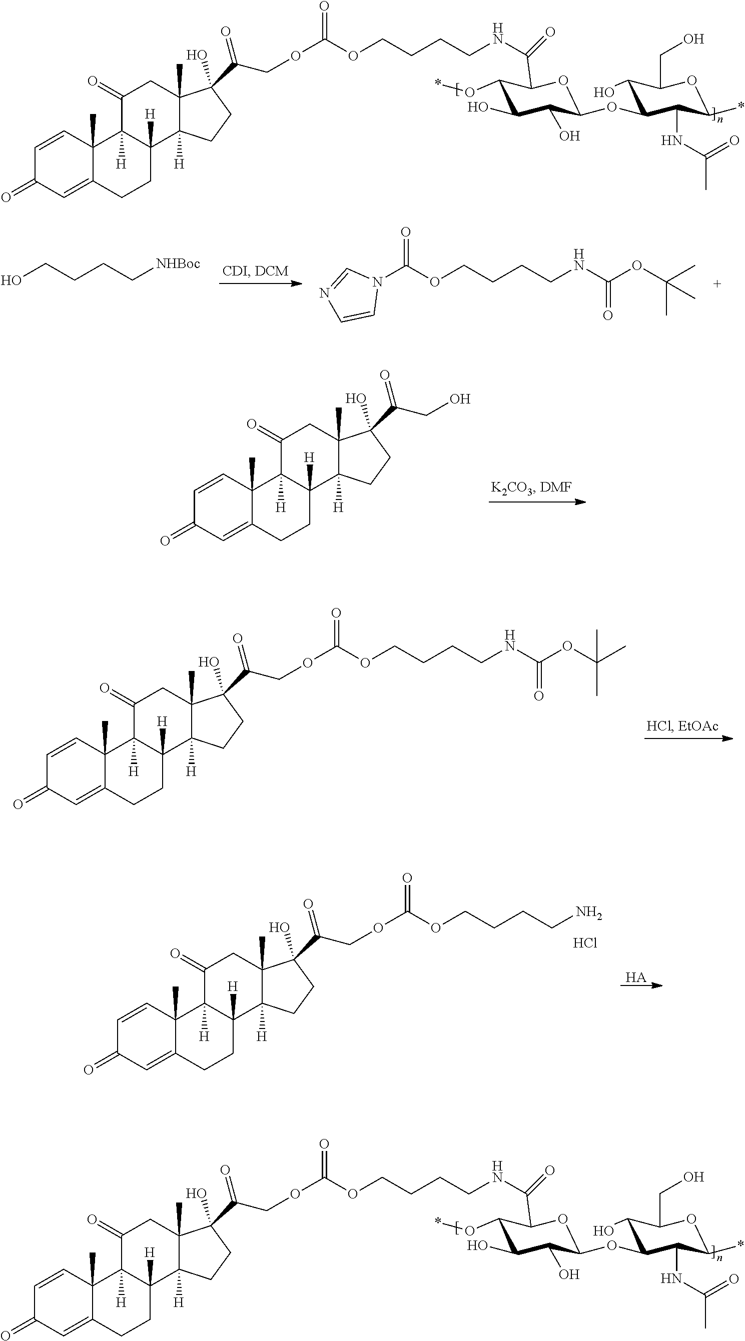

### Step 1: Preparation of 4-((tert-butoxycarbonyl)amino)butyl 1H-imidazole-1-carboxylate

The title compound was prepared in analogy to example 20, step 1 by using tert-butyl (4-hydroxybutyl)carbamate instead of tert-butyl (2-hydroxypropyl)carbamate was obtained as a white solid (10 g, yield: 95.4%). MS (m/z): [M+H]⁺ calcd for C₁₃H₂₁N₃O₄, 284.15; found, 284.2.

### Step 2: Preparation of tert-butyl (4-(((2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethoxy)carbonyl)oxy)butyl)carbamate

To a solution of (8S,9S,10R,13S,14S,17R)-17-hydroxy-17-(2-hydroxyacetyl)-10,13-dimethyl-7,8,9,10,12,13,14,15,16,17-decahydro-3H-cyclopenta[a]phenanthrene-3,11(6H)-dione (600 mg, 1.67 mmol) in DMF (20 mL) was added 4-((tert-butoxycarbonyl)amino)butyl 1H-imidazole-1-carboxylate (569 mg, 2 mmol) and K₂CO₃ (1298 mg, 8.37 mmol) under N₂. The resulting mixture was stirred at room temperature for 16 hours. The reaction mixture was poured into water (80 mL) at room temperature, filtered and the filter cake was dissolved in EtOAc(50 mL×3). The solution was dried over sodium sulfate, filtered, and then concentrated under reduced pressure. The residue was purified by silical gel chromatography to give the title product (0.8 g, yield: 83.5%); MS (m/z): [M+H]⁺ calcd for C₃₁H₄₃NO₉, 574.29; found, 474.2 (M+H−100). ¹FH NMR (400 MHz, DMSO) δ 7.60 (t, J=10.0 Hz, 1H), 6.81 (dd, J=19.6, 14.2 Hz, 1H), 6.12 (dd, J=10.3, 1.9 Hz, 1H), 6.03 (d, J=9.5 Hz, 1H), 5.84 (s, 1H), 4.98 (d, J=17.9 Hz, 1H), 4.80 (d, J=17.9 Hz, 1H), 4.21-4.00 (m, 2H), 3.44-3.35 (m, 2H), 3.03-2.85 (m, 2H), 2.57 (s, 1H), 2.42-2.26 (m, 2H), 2.25-2.15 (m, 2H), 2.14-1.93 (m, 2H), 1.85-1.72 (m, 1H), 1.71-1.53 (m, 3H), 1.52-1.32 (m, 15H), 1.31-1.17 (m, 1H), 0.53 (s, 3H).

### Step 3: Preparation of 4-aminobutyl (2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl) carbonate hydrochloride

The title compound was prepared in analogy to example 3, step 2 by using tert-butyl (4-(((2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethoxy)carbonyl)oxy)butyl)carbamate instead of tert-butyl (4-(((2-((6αS,6βR,7S,8αS,8βS,11αR,12αS,12βS)-6β-fluoro-7-hydroxy-6α,8α,10,10-tetramethyl-4-oxo-1,2,4,6α,6β,7,8,8α,11α,12,12α,12b-dodecahydro-8βH-naphtho[2′,1′:4,5]indeno[1,2-d][1,3]dioxol-8β-yl)-2-oxoethoxy)carbonyl)oxy)butyl)carbamate was obtained as a white solid (0.66 g, yield: 99%). MS (m/z): [M+H]⁺ calcd for C₂₆H₃₅NO₇, 474.24; found, 474.2. ¹H NMR (400 MHz, DMSO) δ 8.03 (s, 4H), 7.59 (t, J=9.4 Hz, 1H), 6.11 (dd, J=10.3, 1.8 Hz, 1H), 6.02 (s, 2H), 5.03 (d, J=17.9 Hz, 1H), 4.81 (d, J=17.9 Hz, 1H), 4.17-4.08 (m, 2H), 2.89 (t, J=14.7 Hz, 1), 2.85-2.72 (m, 3H), 2.38 (dd, J=20.2, 9.0 Hz, 2H), 2.19 (dd, J=11.7, 6.7 Hz, 2H), 2.11-1.96 (m, 3H), 1.82-1.62 (m, 6H), 1.42-1.32 (m, 4H), 1.24 (s, 1H), 0.52 (s, 3H).

### Step 4: Preparation of Conjugate of HA and 4-aminobutyl (2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl) carbonate

The title compound was prepared in analogy to example 34, step 3 by using 4-aminobutyl (2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.228 g, yield: 67.8%, DSR=7%). ¹H NMR (400 MHz, D₂O) δ 7.88 (d, J=10.3 Hz, 0.07H), 6.31 (d, J=9.7 Hz, 0.07H), 6.23 (s, 0.07H), 5.22-4.93 (s, 0.14H), 4.78-4.30 (m, 2H), 4.29-2.79 (m, 10.42H), 2.77-2.13 (m, 0.28H), 2.12-1.52 (m, 3.63H), 1.47 (s, 0.21H), 1.46-1.11 (m, 0.14H), 0.69 (s, 0.21H).

With this procedure, reaction of sodium hyaluronate (MW 50 KDa) provided corresponding product (0.07 g, yield: 20.8%, DSR=10%). ¹H NMR (400 MHz, D₂O) δ 7.89 (d, J=10.1 Hz, 0.1H), 6.30 (s, 0.1H), 6.24 (s, 0.1H), 5.24-4.94 (m, 0.2H), 4.69-4.40 (m, 2H), 4.35-2.84 (m, 10.6H), 2.74-2.16 (m, 0.4H), 2.15-1.54 (m, 3.9H), 1.48 (s, 0.3H), 1.46-1.11 (m, 0.2H), 0.70 (s, 0.3H).

### Example 61

### Preparation of Conjugate of CS and 4-aminobutyl (2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl) carbonate

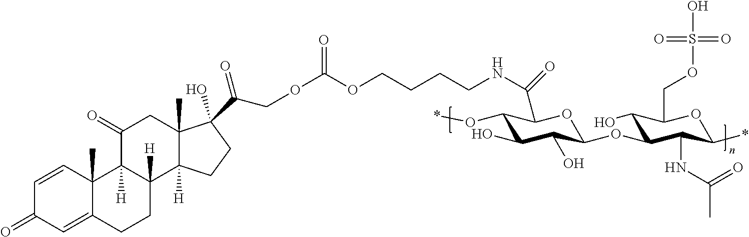

The title compound was prepared in analogy to example 60, step 4 by using CS (Chondroitin sulfate) instead of HA (Hyaluronic acid) was obtained as a white solid (0.186 g, yield: 62%, DSR=10%). ¹H NMR (400 MHz, D₂O) δ 7.87 (s, 0.1H), 6.31 (d, J=10.8 Hz, 0.1H), 6.23 (s, 0.1H), 5.23-4.95 (m, 0.2H), 4.70-4.41 (m, 2H), 4.33-3.07 (m, 10.6H), 2.92-2.15 (m, 0.4H), 2.14-1.51 (m, 3.9H), 1.47 (s, 0.3H), 1.41-0.85 (m, 0.2H), 0.69 (s, 0.3H).

### Example 62

### Preparation of Conjugate of HA and 4-aminobutyl (2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl) carbonate

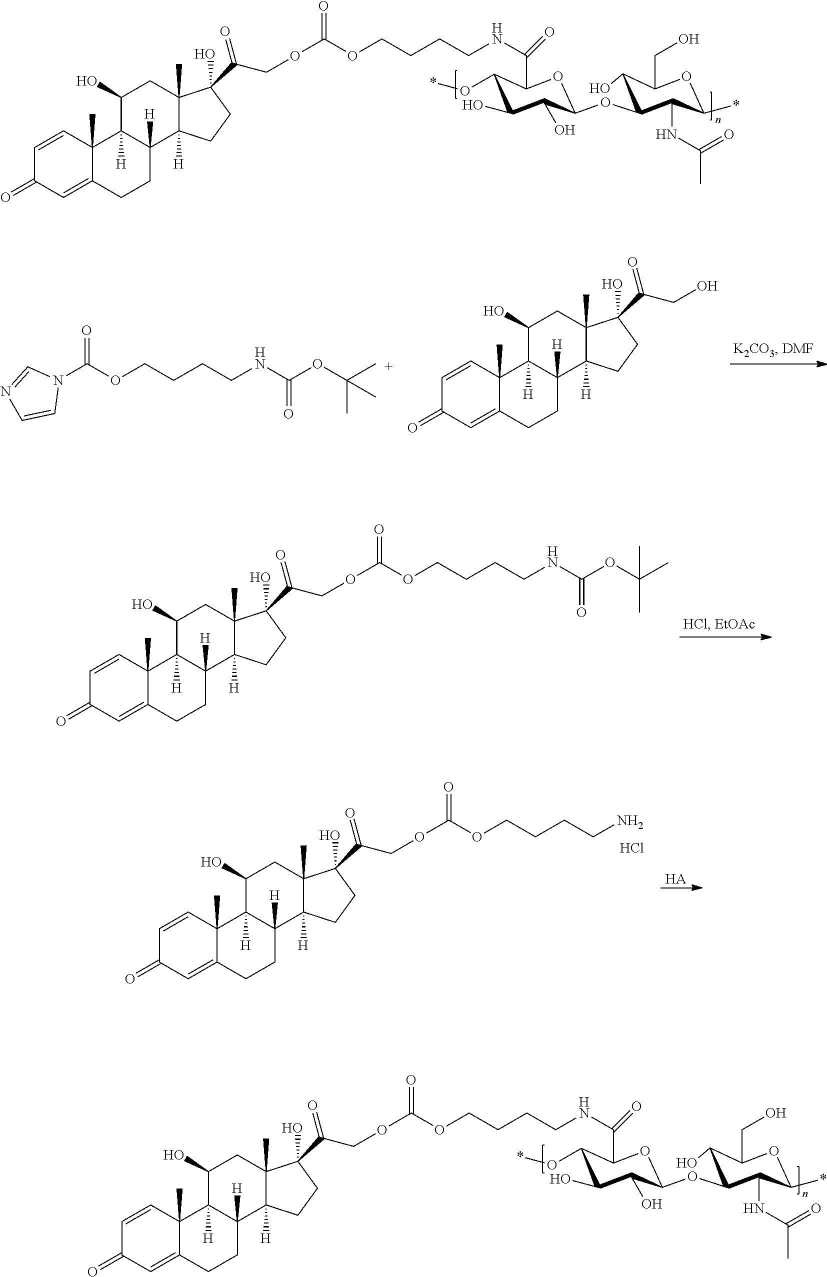

### Step 1: Preparation of tert-butyl (4-(((2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethoxy)carbonyl)oxy)butyl)carbamate

The title compound was prepared in analogy to example 60, step 2 by using (8S,9S,10R,11S,13S,14S,17R)-11,17-dihydroxy-17-(2-hydroxyacetyl)-10,13-dimethyl-6,7,8,9,10,11,12,13,14,15,16,17-dodecahydro-3H-cyclopenta[a]phenanthren-3-one instead of (8S,9S,10R,13S,14S,17R)-17-hydroxy-17-(2-hydroxyacetyl)-10,13-dimethyl-7,8,9,10,12,13,14,15,16,17-decahydro-3H-cyclopenta[a]phenanthrene-3,11(6H)-dione was obtained as a white solid (0.8 g, yield: 83.4%). MS (m/z): [M+H]⁺ calcd for C₃₁H₄₅NO₉, 576.31; found, 598.2 [M+Na]⁺. ¹H NMR (400 MHz, DMSO) δ 7.33 (d, J=10.1 Hz, 1H), 6.83 (t, J=5.5 Hz, 1H), 6.18 (dd, J=10.1, 1.6 Hz, 1H), 5.83 (s, 1H), 5.42 (s, 1H), 5.08 (d, J=17.7 Hz, 1H), 4.77 (dd, J=28.3, 13.2 Hz, 2H), 4.29 (s, 1H), 4.15-3.99 (m, 2H), 3.41-3.33 (m, 2H), 3.03-2.81 (m, 1H), 2.70-2.56 (m, 1H), 2.50-2.42 (m, 1H), 2.19-2.01 (m, 2H), 1.88 (dd, J=13.3, 3.1 Hz, 1H), 1.70-1.52 (m, 5H), 1.51-1.25 (m, 16H), 0.88-0.61 (m, 5H).

### Step 2: Preparation of 4-aminobutyl (2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl) carbonate hydrochloride

The title compound was prepared in analogy to example 60, step 3 by using tert-butyl (4-(((2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethoxy)carbonyl)oxy)butyl)carbamate instead of tert-butyl (4-(((2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethoxy)carbonyl)oxy)butyl)carbamate was obtained as a white solid (0.65 g, yield: 98%). MS (m/z): [M+H]⁺ calcd for C₂₆H₃₇NO₇, 476.26; found, 476.2.

### Step 3: Preparation of Conjugate of HA and 4-aminobutyl (2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl) carbonate

The title compound was prepared in analogy to example 34, step 3 by using 4-aminobutyl (2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.176 g, yield: 58.6%, DSR=9%). ¹H NMR (400 MHz, D₂O) δ 7.62 (s, 0.09H), 6.41-6.35 (m, 0.09H), 6.13 (s, 0.09H), 5.19-5.03 (m, 0.18H), 4.71-4.34 (m, 2H), 4.30-2.95 (m, 10.18H), 2.84-2.16 (m, 0.45H), 2.15-1.40 (m, 4.35H), 0.94-0.85 (m, 0.45H).

With this procedure, reaction of sodium hyaluronate (MW 50 KDa) provided corresponding product (0.209 g, yield: 69.6%, DSR=10%). ¹H NMR (400 MHz, D₂O) δ 7.62 (d, J=10.0 Hz, 0.1H), 6.40 (d, J=9.5 Hz, 0.1H), 6.15 (s, 0.1H), 5.12-4.99 (m, 0.2H), 4.65-4.38 (m, 2H), 4.35-2.86 (m, 10.2H), 2.85-2.34 (m, 0.5H), 2.33-1.42 (m, 4.5H), 0.96-0.84 (m, 0.5H).

### Example 63

### Preparation of Conjugate of CS and 4-aminobutyl (2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl) carbonate

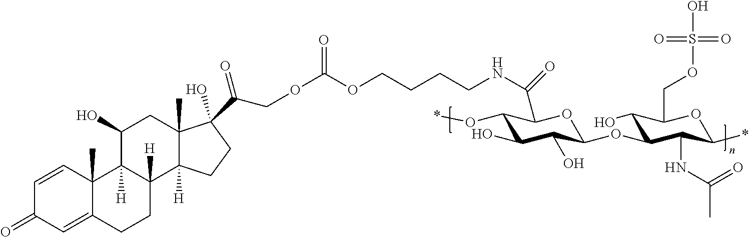

The title compound was prepared in analogy to example 62, step 3 by using CS (Chondroitin sulfate) instead of HA (Hyaluronic acid) was obtained as a white solid (0.213 g, yield: 71%, DSR=6%). ¹FH NMR (400 MHz, D₂O) δ 7.61 (s, 0.06H), 6.39 (d, J=11.0 Hz, 0.06H), 6.13 (s, 0.06H), 5.25-4.99 (m, 0.12H), 4.69-4.39 (m, 2H), 4.38-2.98 (m, 10.12H), 2.97-2.35 (m, 0.3H), 2.34-1.33 (m, 3.9H), 0.99-0.82 (m, 0.3H)

### Example 64

### Preparation of Conjugate of HA and 4-aminobutyl (2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl) carbonate

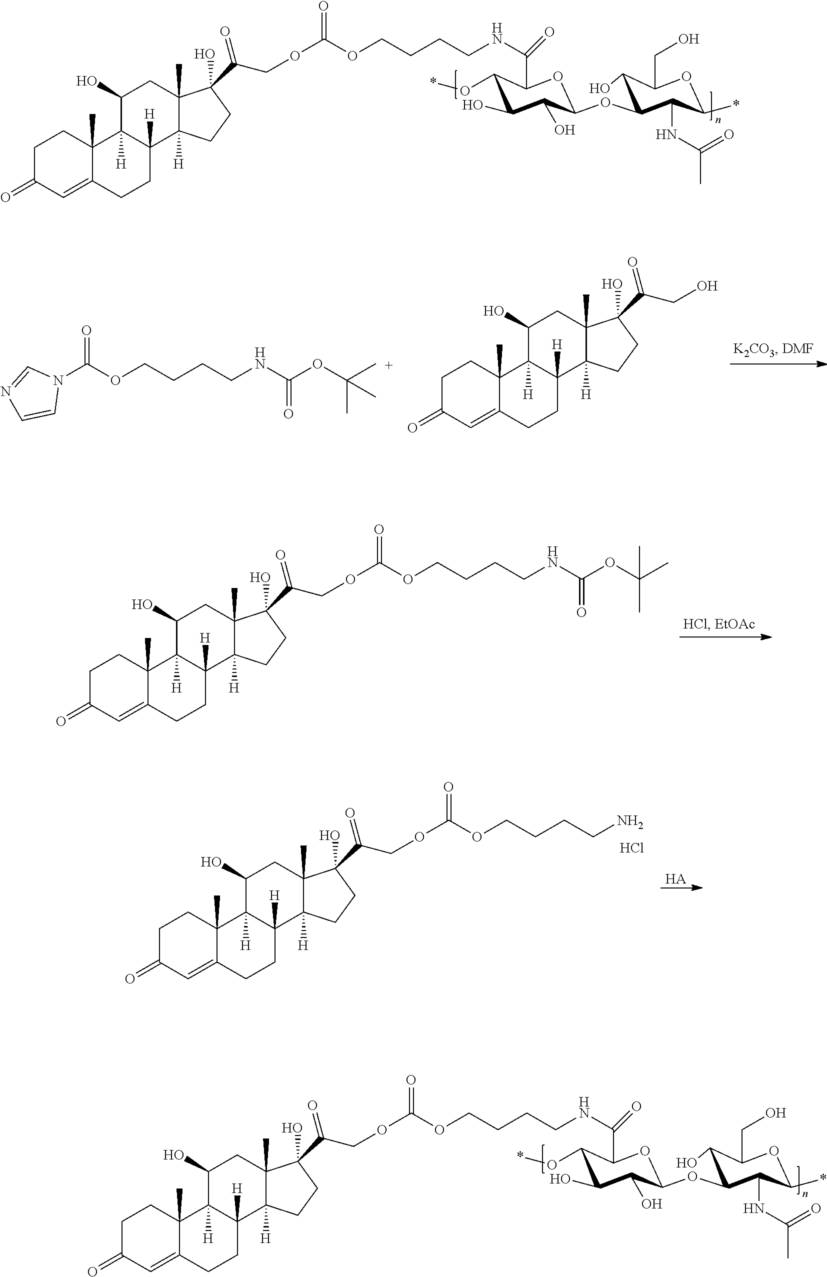

### Step 1: Preparation of tert-butyl (4-(((2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethoxy)carbonyl)oxy)butyl)carbamate

The title compound was prepared in analogy to example 60, step 2 by using (8S,9S,10R,11S,13S,14S,17R)-11,17-dihydroxy-17-(2-hydroxyacetyl)-10,13-dimethyl-1,2,6,7,8,9,10,11,12,13,14,15,16,17-tetradecahydro-3H-cyclopenta[a]phenanthren-3-one instead of (8S,9S,10R,13S,14S,17R)-17-hydroxy-17-(2-hydroxyacetyl)-10,13-dimethyl-7,8,9,10,12,13,14,15,16,17-decahydro-3H-cyclopenta[a]phenanthrene-3,11(6H)-dione was obtained as a white solid (0.7 g, yield: 87.7%). MS (m/z): [M+H]⁺ calcd for C₃₁H₄₇NO₉, 578.33; found, 600.2 [M+Na]⁺.

### Step 2: Preparation of 4-aminobutyl (2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl) carbonate hydrochloride

The title compound was prepared in analogy to example 60, step 3 by using tert-butyl (4-(((2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethoxy)carbonyl)oxy)butyl)carbamate instead of tert-butyl (4-(((2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethoxy)carbonyl)oxy)butyl)carbamate was obtained as a white solid (0.57 g, yield: 98.9%). MS (m/z): [M+H]⁺ calcd for C₂₆H₃₉NO₇, 478.27; found, 478.2. ¹H NMR (400 MHz, DMSO) δ 7.98 (s, 2H), 5.57 (s, 1H), 5.50 (s, 1H), 5.13 (d, J=17.7 Hz, 1H), 4.77 (d, J=17.7 Hz, 1H), 4.40 (d, J=3.9 Hz, 1H), 4.27 (s, 1H), 4.13 (t, J=5.9 Hz, 2H), 2.81 (m, 3H), 2.47-2.29 (m, 2H), 2.25-2.08 (m, 3H), 1.98-1.84 (m, 3H), 1.86-1.55 (m, 8H), 1.54-1.43 (m, 1H), 1.42-1.34 (m, 3H), 1.33-1.23 (m, 1H), 1.09-0.95 (m, 1H), 0.94-0.84 (m, 1H), 0.83-0.70 (m, 3H).

### Step 3: Preparation of Conjugate of HA and 4-aminobutyl (2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl) carbonate

The title compound was prepared in analogy to example 34, step 3 by using 4-aminobutyl (2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.22 g, yield: 73.3%, DSR=10%). ¹H NMR (400 MHz, D₂O) δ 5.87 (s, 0.1H), 5.32-5.04 (m, 0.2H), 4.78-4.29 (m, 2.1H), 4.21-2.91 (m, 10.4H), 2.78-2.22 (m, 0.5H), 2.21-1.58 (m, 4.2H), 1.53-1.46 (m, 0.3H), 1.42-1.18 (m, 0.3H), 1.01-0.89 (m, 0.3H).

With this procedure, reaction of sodium hyaluronate (MW 50 KDa) provided corresponding product (0.23 g, yield: 76.6%, DSR=8%). ¹H NMR (400 MHz, D₂O) δ 5.80 (s, 0.08H), 5.28-4.97 (m, 0.16H), 4.67-4.24 (m, 2.08H), 4.15-2.87 (m, 10.32H), 2.72-2.18 (m, 0.4H), 2.17-1.48 (m, 3.96H), 1.46-1.40 (m, 0.24H), 1.26-1.09 (m, 0.24H), 0.94-0.86 (m, 0.24H).

### Example 65

### Preparation of Conjugate of CS and 4-aminobutyl (2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl) carbonate

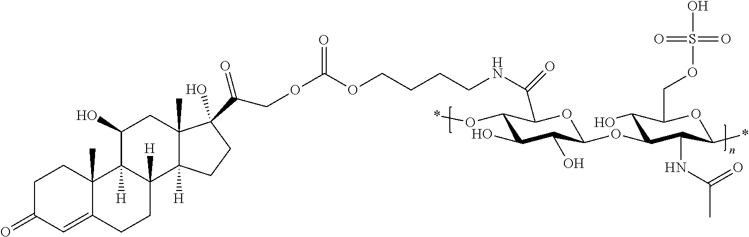

The title compound was prepared in analogy to example 64, step 3 by using CS (Chondroitin sulfate) instead of HA (Hyaluronic acid) was obtained as a white solid (0.436 g, yield: 72.6%, DSR=11%). ¹H NMR (400 MHz, D₂O) δ 5.80 (s, 0.11H), 5.25-4.97 (m, 0.22H), 4.66-4.36 (m, 2.11H), 4.32-2.91 (m, 10.44H), 2.87-2.27 (m, 0.55H), 2.24-1.53 (m, 4.32H), 1.47-1.39 (m, 0.33H), 1.24-1.10 (m, 0.33H), 0.99-0.84 (m, 0.33H).

### Example 66

### Preparation of Conjugate of HA and 4-aminobutyl (2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl) carbonate

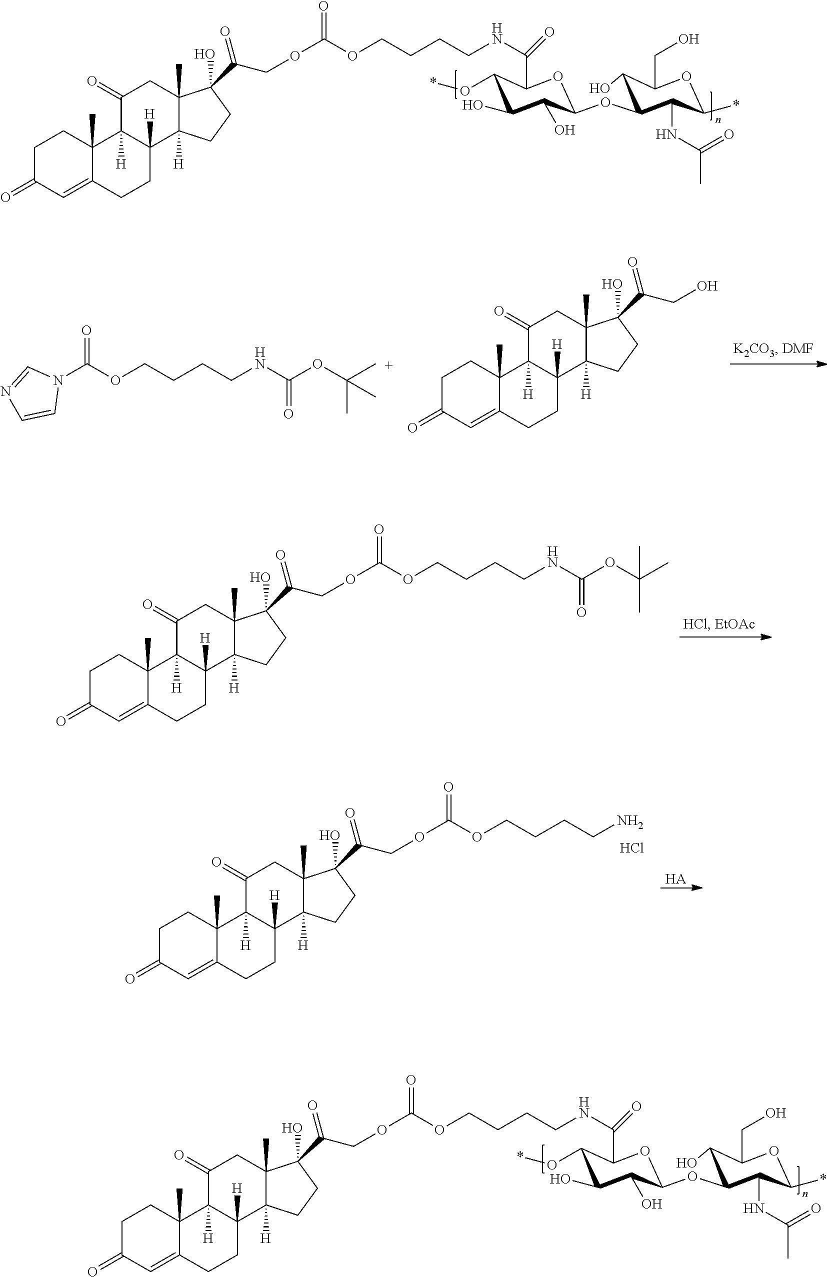

### Step 1: Preparation of tert-butyl (4-(((2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethoxy)carbonyl)oxy)butyl)carbamate

The title compound was prepared in analogy to example 60, step 2 by using (8S,9S,10R,13S,14S,17R)-17-hydroxy-17-(2-hydroxyacetyl)-10,13-dimethyl-1,6,7,8,9,10,12,13,14,15,16,17-dodecahydro-3H-cyclopenta[a]phenanthrene-3,11(2H)-dione instead of (8S,9S,10R,13S,14S,17R)-17-hydroxy-17-(2-hydroxyacetyl)-10,13-dimethyl-7,8,9,10,12,13,14,15,16,17-decahydro-3H-cyclopenta[a]phenanthrene-3,11(6H)-dione was obtained as a white solid (0.6 g, yield: 75%). MS (m/z): [M+H]⁺ calcd for C₃₁H₄₅NO₉, 576.31; found, 598.2 [M+Na]⁺. ¹H NMR (400 MHz, DMSO) δ 6.83-6.79 (m, 1H), 5.84 (s, 1H), 5.65 (d, J=9.2 Hz, 1H), 4.98 (d, J=17.9 Hz, 1H), 4.80 (d, J=17.9 Hz, 1H), 4.09 (t, J=6.5 Hz, 2H), 3.38 (t, J=5.6 Hz, 2H), 2.98-2.87 (m, 1H), 2.61-2.53 (m, 1H), 2.48-2.33 (m, 3H), 2.31-2.21 (m, 1H), 2.19-2.07 (m, 3H), 1.96-1.75 (m, 3H), 1.73-1.52 (m, 3H), 1.47-1.35 (m, 18H), 0.49 (s, 3H).

### Step 2: Preparation of 4-aminobutyl (2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl) carbonate hydrochloride

The title compound was prepared in analogy to example 60, step 3 by using tert-butyl (4-(((2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethoxy)carbonyl)oxy)butyl)carbamate instead of tert-butyl (4-(((2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethoxy)carbonyl)oxy)butyl)carbamate was obtained as a white solid (0.5 g, yield: 97%). MS (m/z): [M+H]⁺ calcd for C₂₆H₃₇NO₇, 476.26; found, 476.3.

### Step 3: Preparation of Conjugate of HA and 4-aminobutyl (2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl) carbonate

The title compound was prepared in analogy to example 34, step 3 by using 4-aminobutyl (2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid (sodium hyaluronate MW 500 KDa 0.196 g, yield: 65.3%, DSR=4%). ¹H NMR (400 MHz, D₂O) δ 5.82 (s, 0.04H), 5.20-4.94 (m, 0.08H), 4.67-4.21 (m, 2.04H), 4.23-3.06 (m, 10.08H), 2.91-2.38 (m, 0.4H), 2.28-1.60 (m, 3.24H), 1.50-1.25 (m, 0.36H), 0.66 (s, 0.12H).

With this procedure, reaction of sodium hyaluronate (MW 50 KDa) provided corresponding product (0.23 g, yield: 76.6%, DSR=10%). ¹H NMR (400 MHz, D₂O) δ 5.83 (s, 0.1H), 5.24-5.02 (m, 0.2H), 4.67-4.21 (m, 2.1H), 4.19-3.04 (m, 10.2H), 3.00-2.25 (m, 1H), 2.21-1.51 (m, 3.6H), 1.47-1.25 (m, 0.9H), 0.65 (s, 0.3H).

### Example 67

### Preparation of Conjugate of CS and 4-aminobutyl (2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl) carbonate

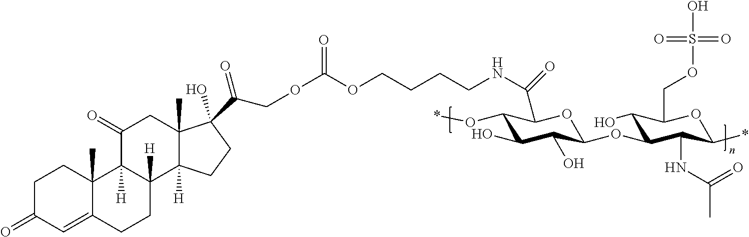

The title compound was prepared in analogy to example 66, step 3 by using CS (Chondroitin sulfate) instead of HA (Hyaluronic acid) was obtained as a white solid (0.433 g, yield: 72.1%, DSR=15%). ¹H NMR (400 MHz, D₂O) δ 5.83 (s, 0.15H), 4.97-4.91 (m, 0.3H), 4.68-4.39 (m, 2.15H), 4.29-3.08 (m, 10.3H), 2.95-2.26 (m, 1.5H), 2.21-1.50 (m, 3.9H), 1.46-1.13 (m, 1.35H), 0.64 (s, 0.45H).

### Example 68

### Preparation of Conjugate of HA and 4-aminobutyl (2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl) carbonate

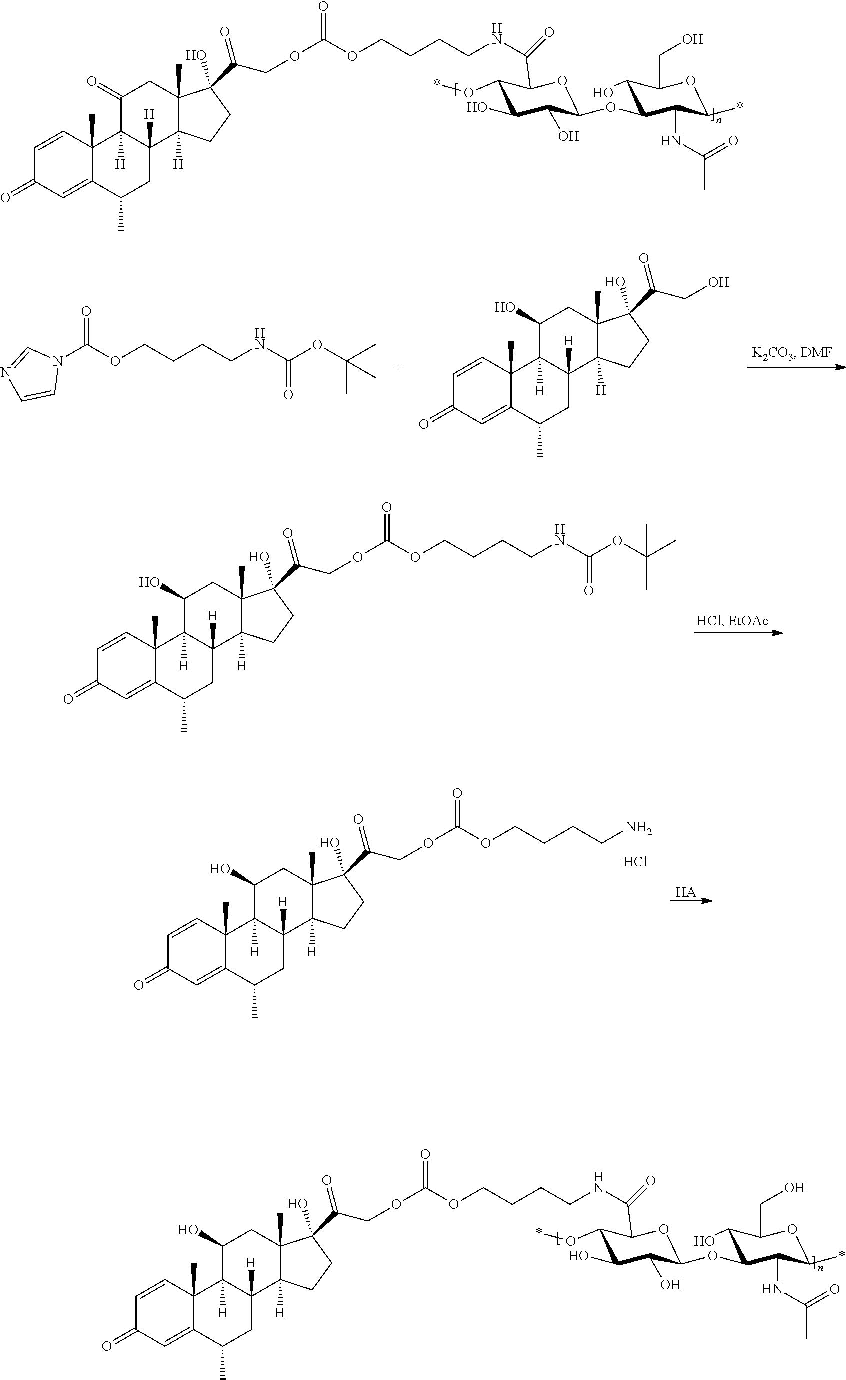

### Step 1: Preparation of tert-butyl (4-(((2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethoxy)carbonyl)oxy)butyl)carbamate

The title compound was prepared in analogy to example 60, step 2 by using (6S,8S,9S,10R,11S,13S,14S,17R)-11,17-dihydroxy-17-(2-hydroxyacetyl)-6,10,13-trimethyl-6,7,8,9,10,11,12,13,14,15,16,17-dodecahydro-3H-cyclopenta[a]phenanthren-3-one instead of (8S,9S,10R,13S,14S,17R)-17-hydroxy-17-(2-hydroxyacetyl)-10,13-dimethyl-7,8,9,10,12,13,14,15,16,17-decahydro-3H-cyclopenta[a]phenanthrene-3,11(6H)-dione was obtained as a white solid (0.7 g, yield: 88.9%). MS (m/z): [M+H]⁺ calcd for C₃₂H₄₇NO₉, 590.33; found, 611.2[M+Na]⁺. ¹H NMR (400 MHz, DMSO) δ 7.33 (d, J=10.1 Hz, 1H), 6.81-6.70 (m, 1H), 6.18 (dd, J=10.1, 1.6 Hz, 1H), 5.83 (s, 1H), 5.42 (s, 1H), 5.08 (d, J=17.7 Hz, 1H), 4.79-4.70 (m, 2H), 4.29 (s, 1H), 4.10 (t, J=6.5 Hz, 2H), 3.44-3.34 (m, 2H), 2.99-2.86 (m, 1H), 2.72-2.60 (m, 1H), 2.17-2.03 (m, 2H), 1.88 (dd, J=13.3, 3.1 Hz, 1H), 1.70-1.52 (m, 5H), 1.51-1.30 (m, 16H), 1.11-1.00 (m, 3H), 0.91-0.67 (m, 5H).

### Step 2: Preparation of 4-aminobutyl (2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl) carbonate hydrochloride

The title compound was prepared in analogy to example 60, step 3 by using tert-butyl (4-(((2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethoxy)carbonyl)oxy)butyl)carbamate instead of tert-butyl (4-(((2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethoxy)carbonyl)oxy)butyl)carbamate was obtained as a white solid (0.55 g, yield: 98%). MS (m/z): [M+H]⁺ calcd for C₂₇H₃₉NO₇, 490.27; found, 490.2.

### Step 3: Preparation of Conjugate of HA and 4-aminobutyl (2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl) carbonate

The title compound was prepared in analogy to example 34, step 3 by using 4-aminobutyl (2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.203 g, yield: 67.6%, DSR=11%). ¹H NMR (400 MHz, D₂O) δ 7.65-7.56 (m, 0.11H), 6.45-6.34 (m, 0.11H), 6.19-6.09 (m, 0.11H), 5.27-5.08 (m, 0.22H), 4.71-4.30 (m, 2.11H), 4.31-2.95 (m, 10.22H), 2.94-2.20 (m, 0.44H), 2.16-1.39 (m, 4.65H), 1.24-1.05 (m, 0.33H), 0.97-0.79 (m, 0.55H).

With this procedure, reaction of sodium hyaluronate (MW 50 KDa) provided corresponding product (0.239 g, yield: 79.6%, DSR=4%). ¹H NMR (400 MHz, D₂O) δ 7.63-7.56 (m, 0.04H), 6.42-6.34 (m, 0.04H), 6.13 (s, 0.04H), 5.27-5.00 (m, 0.08H), 4.68-4.24 (m, 2.04H), 4.20-3.06 (m, 10.08H), 2.83-2.33 (m, 0.16H), 2.32-1.41 (m, 3.6H), 1.15-1.08 (m, 0.12H), 0.94-0.82 (m, 0.2H).

### Example 69

### Preparation of Conjugate of CS and 4-aminobutyl (2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl) carbonate

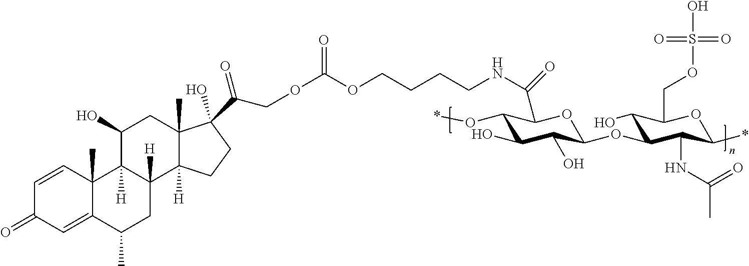

The title compound was prepared in analogy to example 68, step 3 by using CS (Chondroitin sulfate) instead of HA (Hyaluronic acid) was obtained as a white solid (0.521 g, yield: 86.8%, DSR=12%). ¹FH NMR (400 MHz, D₂O) δ 7.66-7.56 (m, 0.12H), 6.45-6.36 (m, 0.12H), 6.10 (s, 0.12H), 5.28-4.96 (m, 0.24H), 4.69-4.37 (m, 2.12H), 4.36-2.99 (m, 10.24H), 2.98-2.35 (m, 0.48H), 2.34-1.53 (m, 4.8H), 1.49-1.31 (m, 0.36H), 1.09-0.83 (m, 0.6H).

### Example 70

### Preparation of Conjugate of HA and 4-aminobutyl (2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate

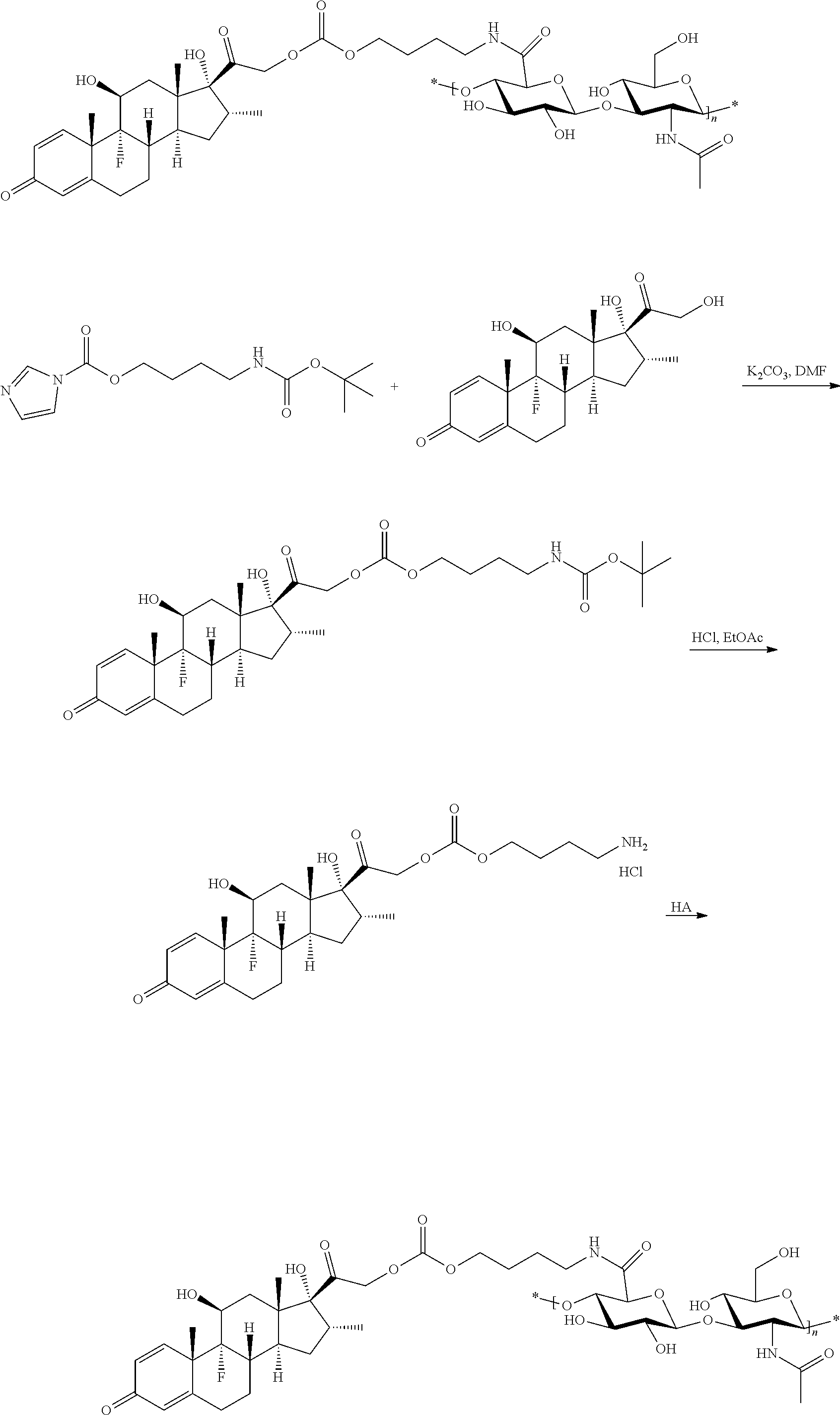

### Step 1: Preparation of tert-butyl (4-(((2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)carbonyl)oxy)butyl)carbamate

The title compound was prepared in analogy to example 60, step 2 by using (8S,9R,10S,11S,135,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one instead of (8S,9S,10R,13S,14S,17R)-17-hydroxy-17-(2-hydroxyacetyl)-10,13-dimethyl-7,8,9,10,12,13,14,15,16,17-decahydro-3H-cyclopenta[a]phenanthrene-3,11(6H)-dione was obtained as a white solid (0.6 g, yield: 77.5%). MS (m/z): [M+H]⁺ calcd for C₃₂H₄₆FNO₉, 608.32; found, 630.2 [M+Na]⁺.

### Step 2: Preparation of 4-aminobutyl (2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate hydrochloride

The title compound was prepared in analogy to example 60, step 3 by using tert-butyl (4-(((2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)carbonyl)oxy)butyl)carbamate instead of tert-butyl (4-(((2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethoxy)carbonyl)oxy)butyl)carbamate was obtained as a white solid (0.48 g, yield: 98%). MS (m/z): [M+H]⁺ calcd for C₂₇H₃₈FNO₇, 508.26; found, 508.2. ¹H NMR (400 MHz, DMSO) δ 7.98 (s, 4H), 7.35 (d, J=10.1 Hz, 1H), 6.22 (dd, J=10.1, 1.8 Hz, 1H), 6.01 (s, 1H), 5.52 (d, J=4.9 Hz, 1H), 5.22 (s, 1H), 5.10 (d, J=17.7 Hz, 1H), 4.79 (d, J=17.7 Hz, 1H), 4.19-3.99 (m, 3H), 2.97-2.74 (m, 2H), 2.62 (td, J=13.4, 6.0 Hz, 1H), 2.45-2.27 (m, 2H), 2.22-2.07 (m, 2H), 1.83-1.74 (m, 1H), 1.73-1.44 (m, 10H), 1.42-1.22 (m, 1H), 1.13-1.03 (m, 1H), 0.90 (s, 3H), 0.80 (d, J=7.2 Hz, 3H).

### Step 3: Preparation of Conjugate of HA and 4-aminobutyl (2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate

The title compound was prepared in analogy to example 34, step 3 by using 4-aminobutyl (2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.196 g, yield: 65.3%, DSR=8%). ¹H NMR (400 MHz, D₂O) δ 7.56 (d, J=10.1 Hz, 0.08H), 6.45 (d, J=9.7 Hz, 0.08H), 6.25 (s, 0.08H), 5.21-4.99 (m, 0.16H), 4.70-4.34 (m, 2.08H), 4.32-2.90 (m, 10.16H), 2.81-2.36 (m, 0.4H), 2.35-1.41 (m, 4.04H), 1.35-1.17 (m, 0.08H), 1.01 (s, 0.24H), 0.94-0.83 (m, 0.24H).

With this procedure, reaction of sodium hyaluronate (MW 50 KDa) provided corresponding product (0.167 g, yield: 55.6%, DSR=11%). ¹H NMR (400 MHz, D₂O) δ 7.57 (d, J=10.7 Hz, 0.11H), 6.46 (d, J=8.5 Hz, 0.11H), 6.26 (s, 0.11H), 5.21-4.96 (m, 0.22H), 4.70-4.39 (m, 2.11H), 4.38-2.87 (m, 10.22H), 2.86-2.34 (m, 0.55H), 2.33-1.39 (m, 4.43H), 1.38-1.12 (m, 0.22H), 1.02 (s, 0.33H), 0.94-0.85 (m, 0.33H).

### Example 71

### Preparation of Conjugate of CS and 4-aminobutyl (2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate

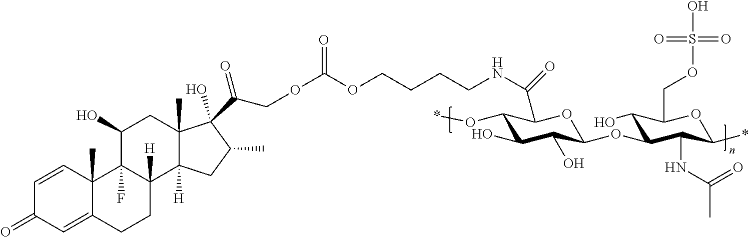

The title compound was prepared in analogy to example 70, step 3 by using CS (Chondroitin sulfate) instead of HA (Hyaluronic acid) was obtained as a white solid (0.456 g, yield: 76%, DSR=24%). ¹H NMR (400 MHz, D₂O) δ 7.56 (d, J=10.1 Hz, 0.24H), 6.44 (d, J=10.4 Hz, 0.24H), 6.24 (s, 0.24H), 5.25-4.94 (m, 0.48H), 4.70-4.37 (m, 2.24H), 4.34-2.90 (m, 10.48H), 2.89-2.22 (m, 1.2H), 2.21-1.37 (m, 6.12H), 1.34-1.16 (m, 0.48H), 1.01 (s, 0.72H), 0.93-0.83 (m, 0.72H).

### Example 72

### Preparation of Conjugate of HA and 4-aminobutyl (2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate

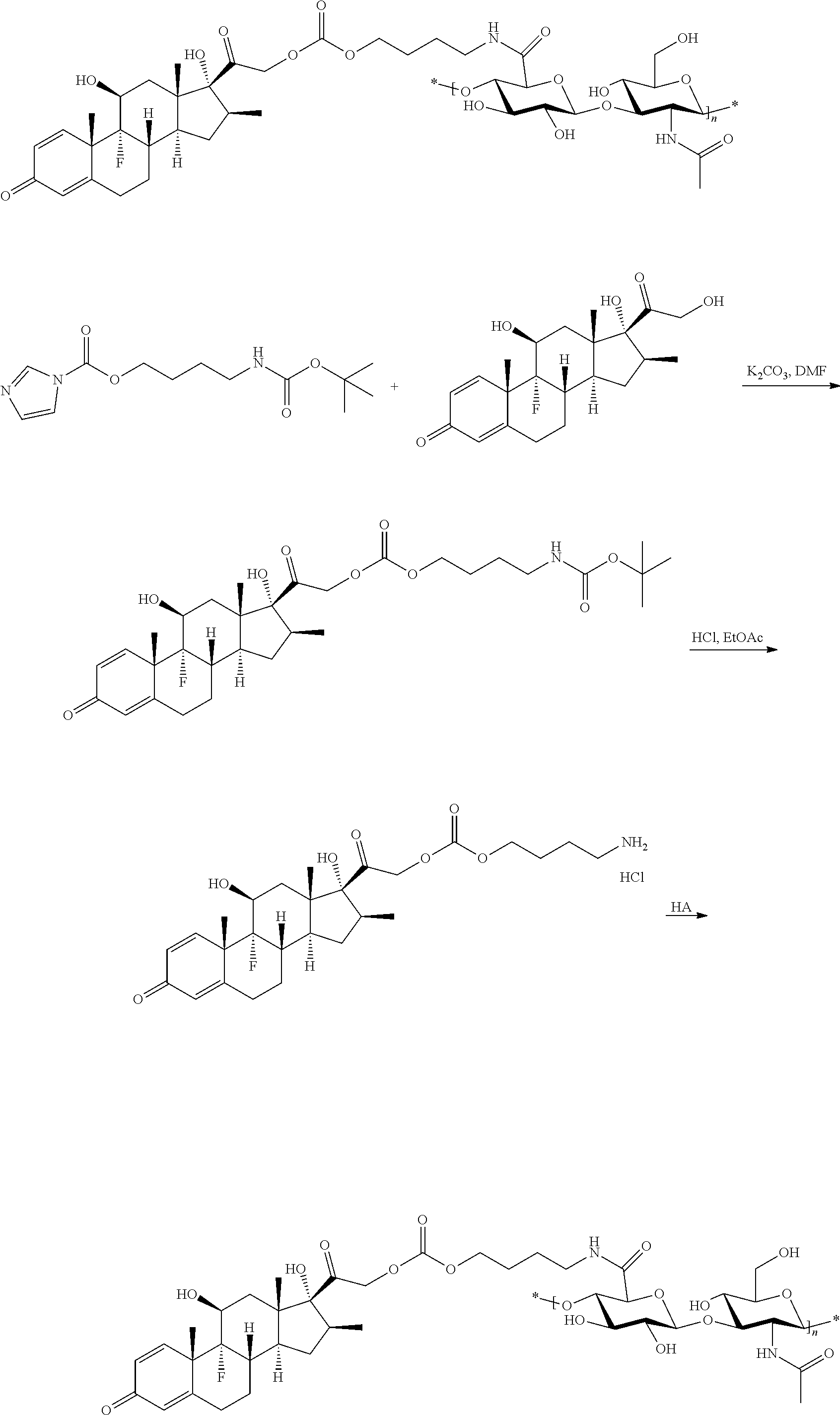

### Step 1: Preparation of tert-butyl (4-(((2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)carbonyl)oxy)butyl)carbamate

The title compound was prepared in analogy to example 60, step 2 by using (8S,9R,10S,11S,13S,14S,16S,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one instead of (8S,9S,10R,13S,14S,17R)-17-hydroxy-17-(2-hydroxyacetyl)-10,13-dimethyl-7,8,9,10,12,13,14,15,16,17-decahydro-3H-cyclopenta[a]phenanthrene-3,11(6H)-dione was obtained as a white solid (0.6 g, yield: 95%). MS (m/z): [M+H]⁺ calcd for C₃₂H₄₆FNO₉, 608.32; found, 629.2 [M+Na]⁺. ¹H NMR (400 MHz, DMSO) δ 7.29 (d, J=10.1 Hz, 1H), 6.81-6.70 (m, 1H), 6.23 (dd, J=10.1, 1.6 Hz, 1H), 6.08-5.89 (m, 1H), 5.42-5.22 (m, 2H), 4.97 (t, J=16.3 Hz, 1H), 4.83-4.74 (m, 1H), 4.24-4.05 (m, 3H), 3.37 (q, J=5.8 Hz, 2H), 2.64 (td, J=13.2, 5.5 Hz, 1H), 2.42-2.27 (m, 1H), 2.12 (dd, J=16.0, 7.7 Hz, 2H), 1.96-1.76 (m, 2H), 1.70-1.14 (m, 20H), 1.10-0.80 (m, 7H).

### Step 2: Preparation of 4-aminobutyl (2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate hydrochloride

The title compound was prepared in analogy to example 60, step 3 by using tert-butyl (4-(((2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)carbonyl)oxy)butyl)carbamate instead of tert-butyl (4-(((2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethoxy)carbonyl)oxy)butyl)carbamate was obtained as a white solid (0.4 g, yield: 98%). MS (m/z): [M+H]⁺ calcd for C₂₇H₃₈FNO₇, 508.26; found, 508.2.

### Step 3: Preparation of Conjugate of HA and 4-aminobutyl (2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate

The title compound was prepared in analogy to example 34, step 3 by using 4-aminobutyl (2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.19 g, yield: 63.3%, DSR=8%). ¹FH NMR (400 MHz, D₂O) δ 7.54 (d, J=10.1 Hz, 0.08H), 6.42 (d, J=9.7 Hz, 0.08H), 6.16 (s, 0.08H), 5.20-4.98 (m, 0.16H), 4.70-4.36 (m, 2.08H), 4.32-3.00 (m, 10.16H), 2.89-2.36 (m, 0.4H), 2.33-1.41 (m, 4.04H), 1.35-1.17 (m, 0.08H), 1.01 (s, 0.24H), 0.94-0.85 (m, 0.24H).

### Example 73

### Preparation of Conjugate of CS and 4-aminobutyl (2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate

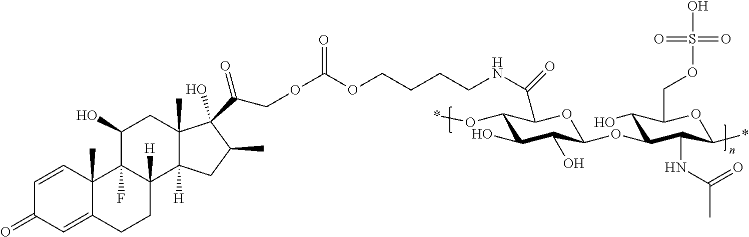

The title compound was prepared in analogy to example 72, step 3 by using CS (Chondroitin sulfate) instead of HA (Hyaluronic acid) was obtained as a white solid (0.202 g, yield: 67.3%, DSR=8%). ¹H NMR (400 MHz, D₂O) δ 7.54 (d, J=10.1 Hz, 0.08H), 6.42 (d, J=9.7 Hz, 0.08H), 6.17 (s, 0.08H), 5.22-4.97 (m, 0.16H), 4.76-4.36 (m, 2.08H), 4.33-3.00 (m, 10.16H), 2.88-2.35 (m, 0.4H), 2.32-1.38 (m, 4.04H), 1.34-1.17 (m, 0.08H), 1.01 (s, 0.24H), 0.94-0.85 (m, 0.24H).

### Example 74

### Preparation of Conjugate of HA and 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 5-aminopentanoate

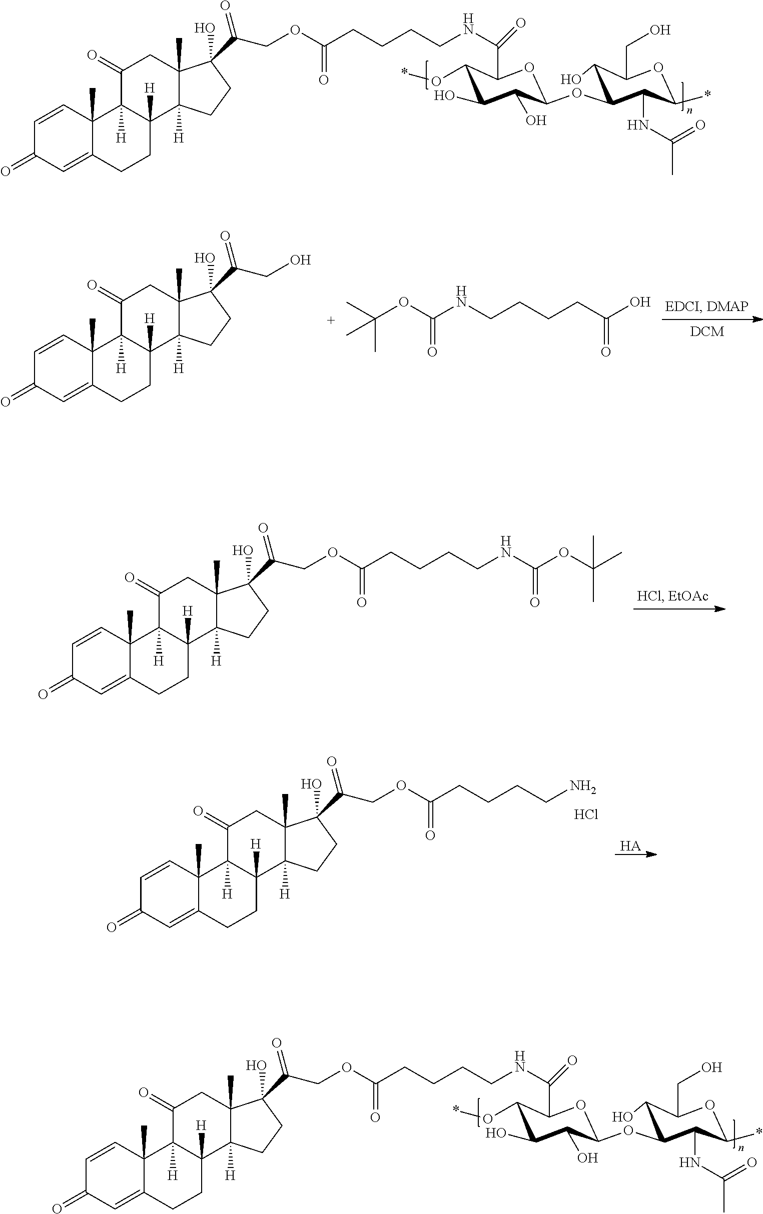

### Step 1: Preparation of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 5-((tert-butoxycarbonyl)amino)pentanoate

The title compound was prepared in analogy to example 24, step 1 by using (8S,9S,10R,13S,14S,17R)-17-hydroxy-17-(2-hydroxyacetyl)-10,13-dimethyl-7,8,9,10,12,13,14,15,16,17-decahydro-3H-cyclopenta[a]phenanthrene-3,11(6H)-dione instead of (6α,9α,11β,16β)-6,9-difluoro-11,21-dihydroxy-16,17-[(1-methylethylidene)bis(oxy)]pregna-1,4-diene-3,20-dione was obtained as a white solid (0.446 g, yield: 71.7%). MS (m/z): [M+H]⁺ calcd for C₃₁H₄₃NO₈, 558.30; found, 580.4 [M+Na]⁺. ¹H NMR (400 MHz, DMSO) δ 7.61 (d, J=10.3 Hz, 1H), 6.78 (t, J=5.2 Hz, 1H), 6.11 (dd, J=10.3, 1.9 Hz, 1H), 6.02 (s, 1H), 5.80 (s, 1H), 4.92 (d, J=17.7 Hz, 1H), 4.80 (d, J=17.7 Hz, 1H), 2.91 (dd, J=13.7, 7.9 Hz, 3H), 2.58-2.52 (m, 3H), 2.42-2.32 (m, 4H), 2.25-2.13 (m, 2H), 2.10-1.93 (m, 2H), 1.82-1.72 (m, 1H), 1.70-1.60 (m, 1H), 1.52 (dd, J=15.0, 7.4 Hz, 2H), 1.45-1.32 (m, 14H), 1.25-1.12 (m, 1H), 0.51 (s, 3H).

### Step 2: Preparation of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 5-aminopentanoate hydrochloride

The title compound was prepared in analogy to example 24, step 2 by using 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 5-aminopentanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.26 g, yield: 96.2%). MS (m/z): [M+H]⁺ calcd for C₂₆H₃₅NO₆, 458.25; found, 458.4.

### Step 3: Preparation of Conjugate of HA and 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 5-aminopentanoate

The title compound was prepared in analogy to example 34, step 3 by using 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 5-aminopentanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.543 g, yield: 86.8%, DSR=34%). ¹H NMR (400 MHz, D₂O) δ 7.90 (d, J=10.0 Hz, 0.34H), 6.32 (d, J=10.4 Hz, 0.34H), 6.24 (s, 0.34H), 5.24-4.88 (m, 0.68H), 4.67-4.43 (m, 2H), 4.14-2.78 (m, 11.02H), 2.75-2.13 (m, 3.06H), 2.07-1.26 (m, 7.08H), 0.70 (s, 1.02H).

### Example 75

### Preparation of Conjugate of HA and 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl-5-aminopentanoate

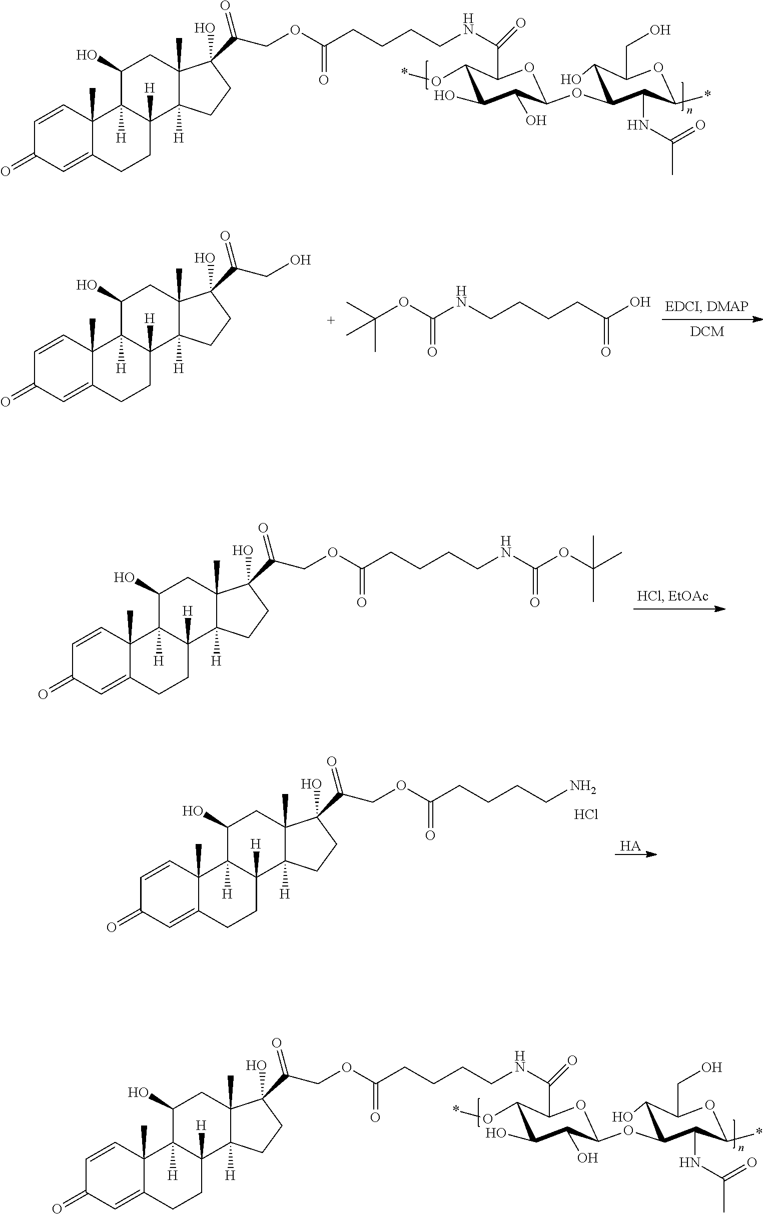

### Step 1: Preparation of 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 5-((tert-butoxycarbonyl)amino)pentanoate

The title compound was prepared in analogy to example 24, step 1 by using (8S,9S,10R,11S,13S,14S,17R)-11,17-dihydroxy-17-(2-hydroxyacetyl)-10,13-dimethyl-6,7,8,9,10,11,12,13,14,15,16,17-dodecahydro-3H-cyclopenta[a]phenanthren-3-one instead of (6α,9α,11β,16β)-6,9-difluoro-11,21-dihydroxy-16,17-[(1-methylethylidene)bis(oxy)]pregna-1,4-diene-3,20-dione was obtained as a white solid (0.556 g, yield: 89.5%). MS (m/z): [M+H]⁺ calcd for C₃₁H₄₅NO₈, 560.31; found, 582.3 [M+Na]⁺. ¹H NMR (400 MHz, DMSO) δ 7.33 (d, J=10.1 Hz, 1H), 6.80 (t, J=5.3 Hz, 1H), 6.17 (dd, J=10.1, 1.7 Hz, 1H), 5.92 (s, 1H), 5.76 (s, 1H), 5.40 (s, 1H), 5.07 (d, J=17.6 Hz, 1H), 4.74 (dd, J=13.3, 10.8 Hz, 2H), 4.29 (s, 1H), 2.92 (q, J=6.5 Hz, 2H), 2.59-2.42 (m, 2H), 2.39 (t, J=7.3 Hz, 2H), 2.34-2.25 (m, 1H), 2.10-1.99 (m, 2H), 1.95-1.82 (m, 1H), 1.73-1.60 (m, 3H), 1.59-1.49 (m, 2H), 1.48-1.22 (m, 15H), 1.10-0.95 (m, 1H), 0.94-0.82 (m, 1H), 0.79 (s, 3H).

### Step 2: Preparation of 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 5-aminopentanoate hydrochloride

The title compound was prepared in analogy to example 24, step 2 by using 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 5-((tert-butoxycarbonyl)amino)pentanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.34 g, yield: 97.7%). MS (m/z): [M+H]⁺ calcd for C₂₆H₃₇NO₆, 460.26; found, 460.2.

### Step 3: Preparation of Conjugate of HA and 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 5-aminopentanoate

The title compound was prepared in analogy to example 34, step 3 by using 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 5-aminopentanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.565 g, yield: 75.3%, DSR=22%). ¹H NMR (400 MHz, D₂O) δ 7.61 (d, J=10.0 Hz, 0.22H), 6.39 (d, J=9.9 Hz, 0.22H), 6.17 (s, 0.22H), 5.20-4.84 (m, 0.66H), 4.70-4.36 (m, 2.22H), 4.28-2.99 (m, 10.44H), 2.75-2.14 (m, 1.1H), 2.13-1.41 (m, 6.08H), 1.19 (d, J=12.2 Hz, 0.44H), 0.91 (s, 0.66H).

### Example 76

### Preparation of Conjugate of HA and 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 5-aminopentanoate

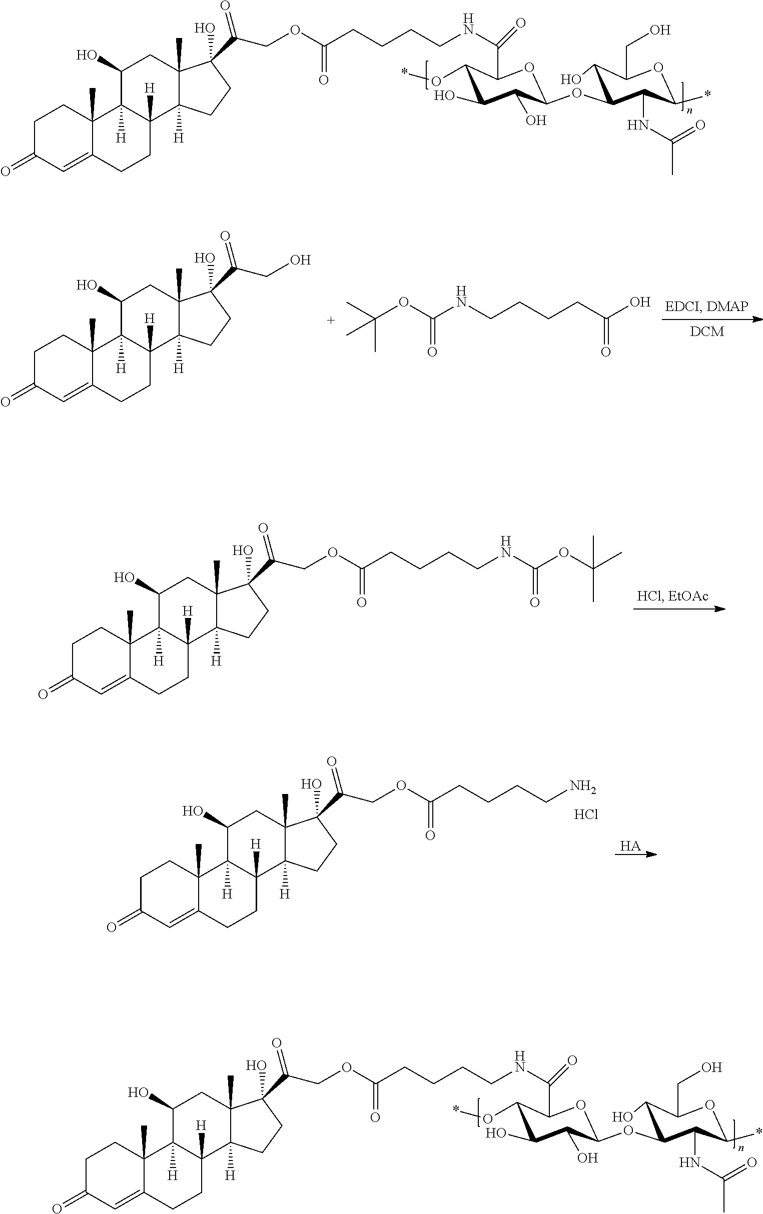

### Step 1: Preparation of 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 5-((tert-butoxycarbonyl)amino)pentanoate

The title compound was prepared in analogy to example 24, step 1 by using (8S,9S,10R,11S,13S,14S,17R)-11,17-dihydroxy-17-(2-hydroxyacetyl)-10,13-dimethyl-1,2,6,7,8,9,10,11,12,13,14,15,16,17-tetradecahydro-3H-cyclopenta[a]phenanthren-3-one instead of (6α,9α,11β,16β)-6,9-difluoro-11,21-dihydroxy-16,17-[(1-methylethylidene)bis(oxy)]pregna-1,4-diene-3,20-dione was obtained as a white solid (0.345 g, yield: 55.5%). MS (m/z): [M+H]⁺ calcd for C₃₁H₄₇NO₈, 562.33; found, 584.3 [M+Na]⁺. ¹H NMR (400 MHz, DMSO) δ 6.78 (s, 1H), 5.56 (s, 1H), 5.38 (s, 1H), 5.08 (d, J=17.5 Hz, 1H), 4.75 (d, J=17.5 Hz, 1H), 4.34 (d, J=3.9 Hz, 1H), 4.27 (s, 1H), 2.91 (q, J=6.6 Hz, 2H), 2.50-2.44 (m, 2H), 2.42-2.31 (m, 3H), 2.25-2.14 (m, 2H), 2.12-2.06 (m, 1H), 1.98-1.85 (m, 3H), 1.84-1.72 (m, 1H), 1.71-1.60 (m, 3H), 1.59-1.49 (m, 2H), 1.48-1.32 (m, 15H), 1.31-1.20 (m, 1H), 1.07-0.91 (m, 1H), 0.87 (dd, J=10.7, 2.3 Hz, 1H), 0.77 (s, 3H).

### Step 2: Preparation of 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 5-aminopentanoate hydrochloride

The title compound was prepared in analogy to example 24, step 2 by using 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 5-((tert-butoxycarbonyl)amino)pentanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.2 g, yield: 94.7%). MS (m/z): [M+H]⁺ calcd for C₂₆H₃₉NO₆, 462.28; found, 462.2. ¹H NMR (400 MHz, D₂O) δ 5.82 (s, 1H), 5.19 (d, J=18.0 Hz, 1H), 5.08 (d, J=17.9 Hz, 1H), 4.54 (d, J=2.7 Hz, 1H), 3.08 (d, J=6.5 Hz, 2H), 2.73-2.55 (m, 5H), 2.47-2.33 (m, 2H), 2.23 (dt, J=13.1, 4.5 Hz, 1H), 2.18-2.07 (m, 2H), 2.06-1.71 (m, 9H), 1.70-1.43 (m, 5H), 1.26-1.08 (m, 2H), 0.89 (s, 3H).

### Step 3: Preparation of Conjugate of HA and 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 5-aminopentanoate

The title compound was prepared in analogy to example 34, step 3 by using 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 5-aminopentanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.205 g, yield: 68.3%, DSR=18%). ¹H NMR (400 MHz, D₂O) δ 5.81 (s, 0.18H), 5.23-4.93 (m, 0.36H), 4.72-4.32 (m, 2.18H), 4.12-2.92 (m, 10.36H), 2.68-2.15 (m, 1.26H), 2.14-1.50 (m, 5.16H), 1.48-0.99 (m, 1.26H), 0.89 (s, 0.54H).

### Example 77

### Preparation of Conjugate of HA and 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 5-aminopentanoate

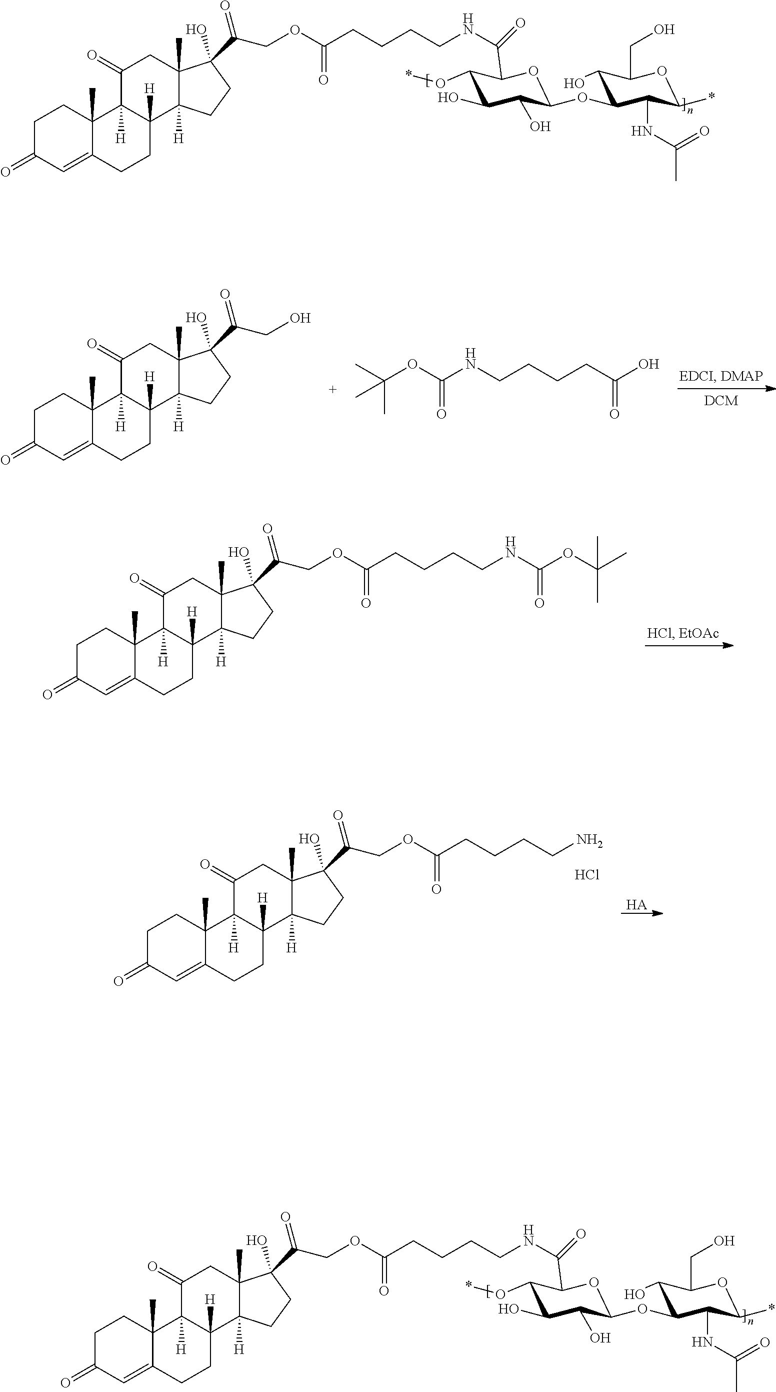

### Step 1: Preparation of 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 5-((tert-butoxycarbonyl)amino)pentanoate

The title compound was prepared in analogy to example 24, step 1 by using (8S,9S,10R,13S,14S,17R)-17-hydroxy-17-(2-hydroxyacetyl)-10,13-dimethyl-1,6,7,8,9,10,12,13,14,15,16,17-dodecahydro-3H-cyclopenta[a]phenanthrene-3,11(2H)-dione instead of (6α,9α,110,160)-6,9-difluoro-11,21-dihydroxy-16,17-[(1-methylethylidene)bis(oxy)]pregna-1,4-diene-3,20-dione was obtained as a white solid (0.5 g, yield: 81.9%). MS (m/z): [M+H]⁺ calcd for C₃₁H₄₅NO₈, 560.31; found, 582.2 [M+Na]⁺. ¹H NMR (400 MHz, DMSO) δ 6.78 (s, 1H), 5.80 (s, 1H), 5.64 (s, 1H), 4.93 (d, J=17.7 Hz, 1H), 4.80 (dd, J=17.7, 5.4 Hz, 1H), 2.95-2.85 (m, 3H), 2.62-2.44 (m, 2H), 2.43-2.32 (m, 4H), 2.31-2.21 (m, 1H), 2.20-2.06 (m, 4H), 1.91 (dt, J=17.6, 9.1 Hz, 2H), 1.83-1.73 (m, 1H), 1.72-1.60 (m, 2H), 1.57-1.46 (m, 2H), 1.45-1.28 (m, 14H), 1.27-1.14 (m, 2H), 0.47 (s, 3H).

### Step 2: Preparation of 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 5-aminopentanoate hydrochloride

The title compound was prepared in analogy to example 24, step 2 by using 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 5-((tert-butoxycarbonyl)amino)pentanoate instead of 2-((2S,6αS,6βR,7S,8αaS,83S,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.25 g, yield: 95.4%). MS (m/z): [M+H]⁺ calcd for C₂₆H₃₇NO₆, 460.26; found, 460.3. ¹H NMR (400 MHz, D₂O) δ 5.86 (s, 1H), 5.14 (d, J=18.1 Hz, 1H), 5.09-4.95 (m, 1H), 3.08 (s, 2H), 2.98 (d, J=12.1 Hz, 1H), 2.74-2.52 (m, 5H), 2.45 (dd, J=19.8, 11.6 Hz, 2H), 2.33 (d, J=12.1 Hz, 3H), 2.20-1.93 (m, 3H), 1.92-1.65 (m, 6H), 1.56 (dt, J=17.3, 9.3 Hz, 1H), 1.50-1.19 (m, 5H), 0.66 (s, 3H).

### Step 3: Preparation of Conjugate of HA and 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 5-aminopentanoate

The title compound was prepared in analogy to example 34, step 3 by using 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 5-aminopentanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid (sodium hyaluronate MW 500 KDa 0.341 g, yield: 83.9%, DSR=7%). ¹H NMR (400 MHz, D2O) δ 5.89-5.84 (m, 0.07H), 5.22-4.93 (m, 0.14H), 4.79-4.29 (m, 2H), 4.22-3.11 (m, 10.21H), 2.83-2.27 (m, 0.7H), 2.26-1.17 (m, 4.05H), 0.68 (s, 0.21H).

### Example 78

### Preparation of Conjugate of HA and 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 5-aminopentanoate

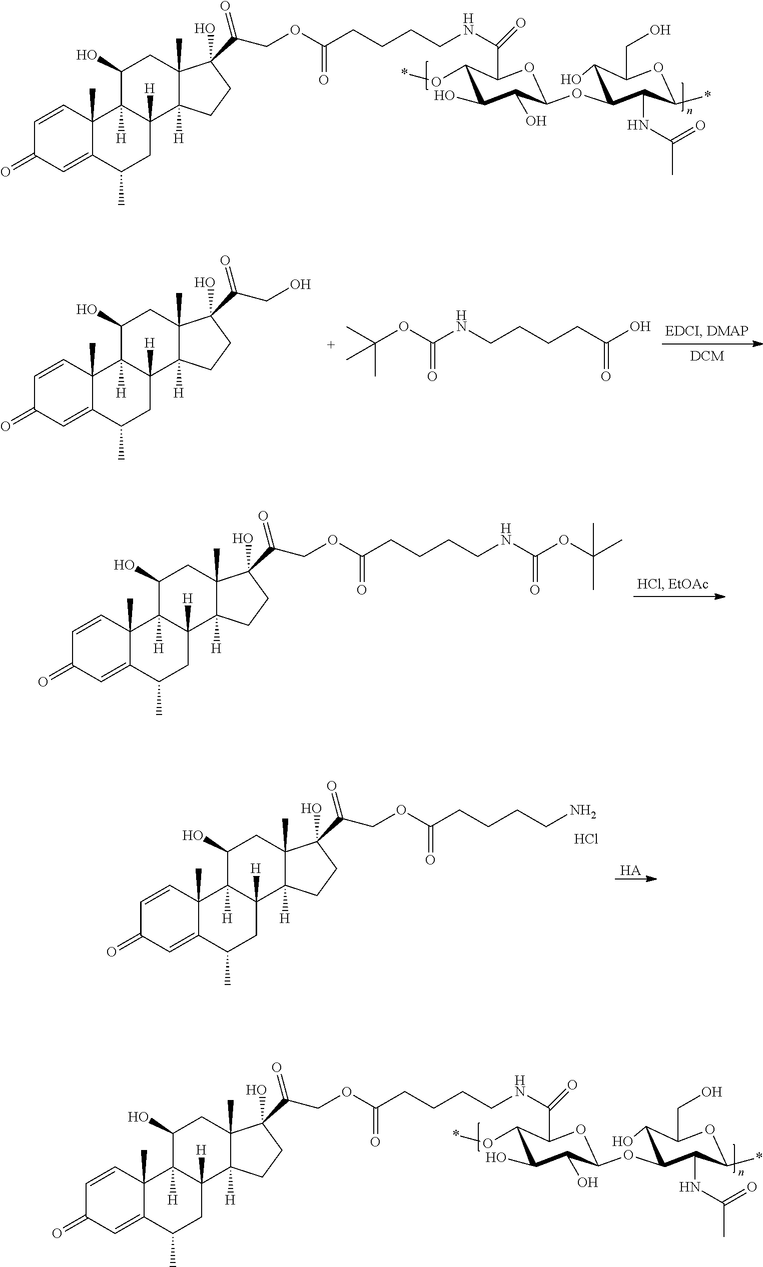

### Step 1: Preparation of 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 5-((tert-butoxycarbonyl)amino)pentanoate

The title compound was prepared in analogy to example 24, step 1 by using (6S,8S,9S,10R,11S,13S,14S,17R)-11,17-dihydroxy-17-(2-hydroxyacetyl)-6,10,13-trimethyl-6,7,8,9,10,11,12,13,14,15,16,17-dodecahydro-3H-cyclopenta[a]phenanthren-3-one instead of (6α,9α,11β,16β)-6,9-difluoro-11,21-dihydroxy-16,17-[(1-methylethylidene)bis(oxy)]pregna-1,4-diene-3,20-dione was obtained as a white solid (0.46 g, yield: 75.3%). MS (m/z): [M+H]⁺ calcd for C₃₂H₄₇NO₈, 574.33; found, 596.2 [M+Na]⁺. ¹H NMR (400 MHz, DMSO) δ 7.33 (d, J=10.1 Hz, 1H), 6.80 (t, J=5.4 Hz, 1H), 6.18 (dd, J=10.1, 1.6 Hz, 1H), 5.82 (s, 1H), 5.75 (s, 2H), 5.39 (s, 1H), 5.07 (d, J=17.6 Hz, 1H), 4.73 (dd, J=13.0, 10.8 Hz, 2H), 4.29 (s, 1H), 2.92 (q, J=6.5 Hz, 2H), 2.74-2.60 (m, 1H), 2.54-2.44 (m, 1H), 2.38 (t, J=7.3 Hz, 2H), 2.17-2.02 (m, 2H), 1.94-1.85 (m, 1H), 1.64 (dd, J=17.5, 9.2 Hz, 3H), 1.59-1.48 (m, 2H), 1.47-1.22 (m, 14H), 1.05 (d, J=6.3 Hz, 3H), 0.86 (dd, J=11.1, 3.0 Hz, 1H), 0.78 (s, 3H), 0.76-0.60 (m, 1H).

### Step 2: Preparation of 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 5-aminopentanoate hydrochloride

The title compound was prepared in analogy to example 24, step 2 by using 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 5-((tert-butoxycarbonyl)amino)pentanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.31 g, yield: 96%). MS (m/z): [M+H]⁺ calcd for C₂₇H₃₉NO₆, 474.28; found, 474.3. ¹H NMR (400 MHz, D₂O) δ 7.61 (d, J=10.0 Hz, 1H), 6.40 (d, J=10.1 Hz, 1H), 6.13 (s, 1H), 5.16 (d, J=18.0 Hz, 1H), 5.05 (d, J=17.9 Hz, 1H), 4.52 (s, 1H), 3.08 (d, J=6.4 Hz, 2H), 2.86-2.75 (m, 1H), 2.62 (d, J=6.3 Hz, 3H), 2.21 (dt, J=23.8, 15.3 Hz, 2H), 1.97 (d, J=11.0 Hz, 1H), 1.80 (dd, J=11.1, 7.9 Hz, 6H), 1.75-1.37 (m, 6H), 1.18 (t, J=9.8 Hz, 3H), 1.07 (dd, J=11.3, 2.5 Hz, 1H), 0.99-0.67 (m, 4H).

### Step 3: Preparation of Conjugate of HA and 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 5-aminopentanoate

The title compound was prepared in analogy to example 34, step 3 by using 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 5-aminopentanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.546 g, yield: 88.3%, DSR=49%). ¹H NMR (400 MHz, D₂O) δ 7.61 (d, J=9.8 Hz, 0.49H), 6.40 (d, J=10.6 Hz, 0.49H), 6.14 (s, 0.49H), 5.15-4.79 (m, 0.98H), 4.71-4.31 (m, 2.49H), 4.26-3.01 (m, 10.98H), 2.87-2.17 (m, 2.94H), 2.16-1.29 (m, 9.37H), 1.16 (d, J=5.7 Hz, 1.96H), 0.90 (s, 1.96H).

### Example 79

### Preparation of Conjugate of HA and 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 5-aminopentanoate

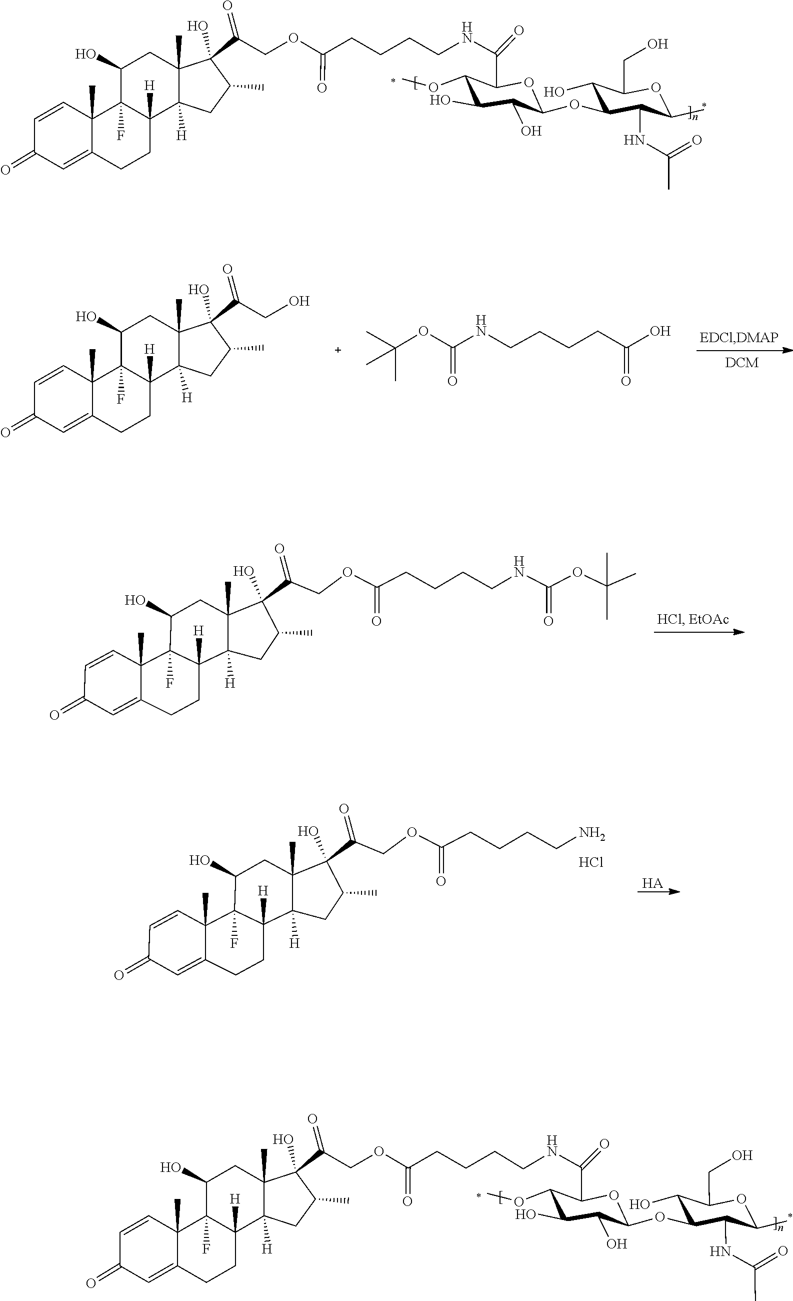

### Step 1: Preparation of 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 5-((tert-butoxycarbonyl)amino)pentanoate

The title compound was prepared in analogy to example 24, step 1 by using (8S,9R,10S,11S,13S,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one instead of (6α,9α,11β,16β)-6,9-difluoro-11,21-dihydroxy-16,17-[(1-methylethylidene)bis(oxy)]pregna-1,4-diene-3,20-dione was obtained as a white solid (0.524 g, yield: 86.9%). MS (m/z): [M+H]⁺ calcd for C₃₂H₄₆FNO₈, 592.32; found, 614.2 [M+Na]⁺. ¹H NMR (400 MHz, DMSO) δ 7.28 (d, J=10.1 Hz, 1H), 6.78 (d, J=5.4 Hz, 1H), 6.22 (dd, J=10.1, 1.7 Hz, 1H), 6.01 (s, 1H), 5.35-5.27 (m, 2H), 4.97 (d, J=17.7 Hz, 1H), 4.81 (d, J=17.7 Hz, 1H), 4.19-4.09 (m, 1H), 2.96-2.85 (m, 2H), 2.63 (td, J=13.2, 5.4 Hz, 1H), 2.44 (dd, J=11.9, 5.0 Hz, 1H), 2.35 (dt, J=13.9, 5.6 Hz, 3H), 2.15-2.03 (m, 2H), 2.02-1.79 (m, 3H), 1.61-1.32 (m, 17H), 1.29-1.19 (m, 1H), 1.10-0.95 (m, 4H), 0.94-0.80 (m, 3H).

### Step 2: Preparation of 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 5-aminopentanoate hydrochloride

The title compound was prepared in analogy to example 24, step 2 by using 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 5-((tert-butoxycarbonyl)amino)pentanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.115 g, yield: 86.4%). MS (m/z): [M+H]⁺ calcd for C₂₇H₃₈FNO₆, 492.27; found, 492.3. ¹H NMR (400 MHz, D₂O) δ 7.57 (d, J=10.1 Hz, 1H), 6.47 (dd, J=10.1, 1.7 Hz, 1H), 6.28 (s, 1H), 5.18-5.02 (m, 2H), 4.44 (d, J=9.0 Hz, 1H), 3.08 (d, J=6.6 Hz, 2H), 2.79 (td, J=13.4, 5.9 Hz, 1H), 2.73-2.46 (m, 4H), 2.32-1.99 (m, 5H), 1.85-1.65 (m, 5H), 1.64-1.48 (m, 4H), 1.33-1.22 (m, 1H), 1.13 (d, J=7.3 Hz, 3H), 1.04 (s, 3H).

### Step 3: Preparation of Conjugate of HA and 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 5-aminopentanoate

The title compound was prepared in analogy to example 34, step 3 by using 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 5-aminopentanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.256 g, yield: 72.9%, DSR=31%). ¹H NMR (400 MHz, D₂O) δ 7.58 (d, J=10.3 Hz, 0.31H), 6.46 (d, J=9.9 Hz, 0.31H), 6.27 (s, 0.31H), 5.09 (s, 0.62H), 4.69-4.32 (m, 2.31H), 4.14-2.91 (m, 10.62H), 2.85-2.30 (m, 1.55H), 2.29-1.77 (m, 4.55H), 1.76-1.19 (m, 3.1H), 1.16-0.84 (m, 1.86H).

### Example 80

### Preparation of Conjugate of HA and 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 5-aminopentanoate

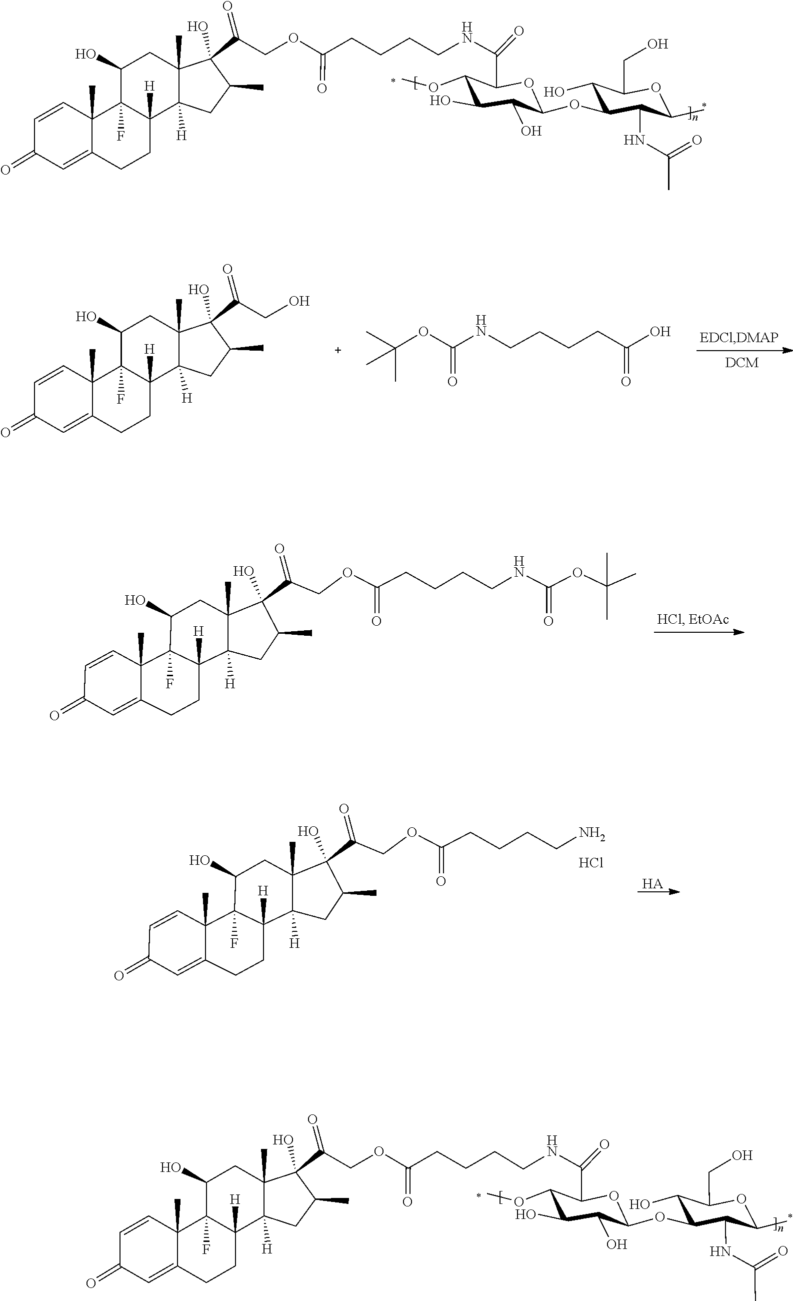

### Step 1: Preparation of 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 5-((tert-butoxycarbonyl)amino)pentanoate

The title compound was prepared in analogy to example 24, step 1 by using (8S,9R,10S,11S,13S,14S,16S,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one instead of (6α,9α,11β,16β)-6,9-difluoro-11,21-dihydroxy-16,17-[(1-methylethylidene)bis(oxy)]pregna-1,4-diene-3,20-dione was obtained as a white solid (0.527 g, yield: 87.4%). MS (m/z): [M+H]⁺ calcd for C₃₂H₄₆FNO₈, 592.32; found, 614.2 [M+Na]⁺. ¹H NMR (400 MHz, DMSO) δ 7.29 (d, J=10.1 Hz, 1H), 6.79 (s, 1H), 6.22 (dd, J=10.1, 1.7 Hz, 1H), 6.01 (s, 1H), 5.38 (d, J=4.3 Hz, 1H), 5.13 (s, 1H), 5.02 (d, J=17.6 Hz, 1H), 4.79 (d, J=17.6 Hz, 1H), 4.19-4.11 (m, 1H), 2.97-2.82 (m, 3H), 2.67-2.57 (m, 1H), 2.43-2.27 (m, 4H), 2.22-2.07 (m, 2H), 1.83-1.72 (m, 1H), 1.70-1.46 (m, 7H), 1.45-1.29 (m, 12H), 1.12-1.01 (m, 1H), 0.87 (d, J=11.8 Hz, 3H), 0.77 (t, J=14.3 Hz, 3H).

### Step 2: Preparation of 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 5-aminopentanoate hydrochloride

The title compound was prepared in analogy to example 24, step 2 by using 2-((8S,9R,105,115,135,145,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 5-((tert-butoxycarbonyl)amino)pentanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.14 g, yield: 93.6%). MS (m/z): [M+H]⁺ calcd for C₂₇H₃₈FNO₆, 492.27; found, 492.2 ¹H NMR (400 MHz, D₂O) δ 7.58 (d, J=10.1 Hz, 1H), 6.47 (d, J=10.2 Hz, 1H), 6.28 (s, 1H), 5.11 (q, J=18.1 Hz, 2H), 4.45 (d, J=8.6 Hz, 1H), 3.09 (s, 2H), 2.84-2.68 (m, 1H), 2.67-2.47 (m, 4H), 2.32-2.14 (m, 2H), 2.06-1.97 (m, 1H), 1.96-1.71 (m, 6H), 1.70-1.39 (m, 4H), 1.34-1.26 (m, 1H), 1.03 (s, 3H), 0.92 (d, J=7.2 Hz, 3H).

### Step 3: Preparation of Conjugate of HA and 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 5-aminopentanoate

The title compound was prepared in analogy to example 34, step 3 by using 2-((8S,9R,10S,11S,135,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 5-aminopentanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.475 g, yield: 83.9%, DSR=24%). ¹H NMR (400 MHz, D₂O) δ 7.58 (d, J=9.9 Hz, 0.24H), 6.46 (d, J=10.3 Hz, 0.24H), 6.26 (s, 0.24H), 5.09 (d, J=8.6 Hz, 0.48H), 4.68-4.23 (m, 2.24H), 4.21-2.90 (m, 10.72H), 2.86-2.37 (m, 1.2H), 2.31-1.77 (m, 5.16H), 1.76-1.17 (m, 1.2H), 1.02 (s, 0.72H), 0.91 (d, J=7.2 Hz, 0.72H).

### Example 81

### Preparation of Conjugate of HA and 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 6-aminohexanoate

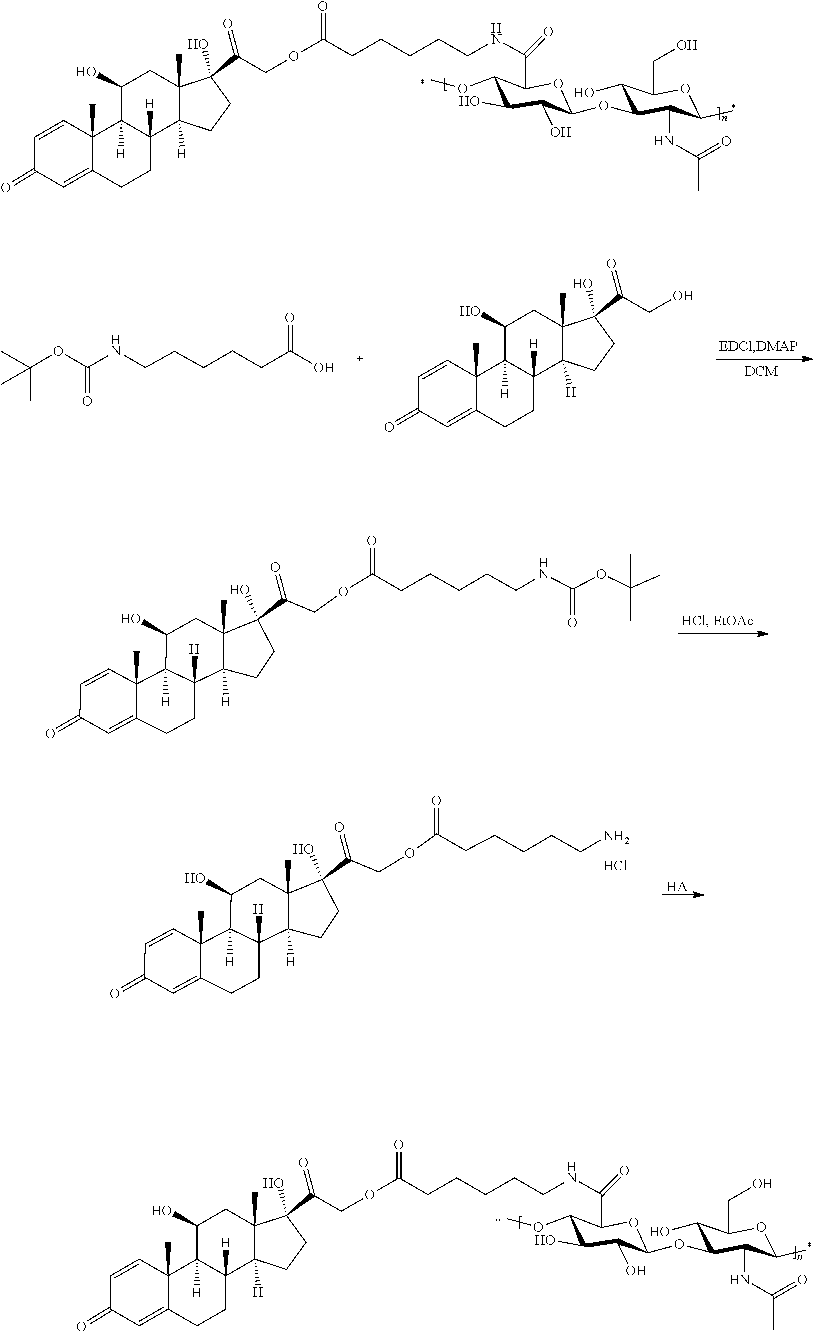

### Step 1: Preparation of 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 6-((tert-butoxycarbonyl)amino)hexanoate

The title compound was prepared in analogy to example 23, step 1 by using (8S,9S,10R,11S,13S,14S,17R)-11,17-dihydroxy-17-(2-hydroxyacetyl)-10,13-dimethyl-6,7,8,9,10,11,12,13,14,15,16,17-dodecahydro-3H-cyclopenta[a]phenanthren-3-one instead of (6α,9α,11β,16β)-6,9-difluoro-11,21-dihydroxy-16,17-[(1-methylethylidene)bis(oxy)]pregna-1,4-diene-3,20-dione was obtained as a white solid (0.436 g, yield: 91.2%). MS (m/z): [M+H]⁺ calcd for C₃₂H₄₇NO₈, 574.33; found, 473.2 (M+H−100). ¹H NMR (400 MHz, DMSO) δ 7.33 (d, J=10.1 Hz, 1H), 6.76 (t, J=5.3 Hz, 1H), 6.17 (dd, J=10.1, 1.7 Hz, 1H), 5.92 (s, 1H), 5.40 (s, 1H), 5.07 (d, J=17.6 Hz, 1H), 4.74 (dd, J=15.0, 10.7 Hz, 2H), 4.32 (d, J=24.0 Hz, 1H), 2.90 (dd, J=12.9, 6.6 Hz, 2H), 2.63-2.43 (m, 2H), 2.42-2.26 (m, 3H), 2.06 (dd, J=19.2, 8.7 Hz, 2H), 1.89 (dd, J=13.2, 2.9 Hz, 1H), 1.72-1.51 (m, 5H), 1.50-1.24 (m, 18H), 1.10-0.98 (m, 1H), 0.97-0.81 (m, 1H), 0.79 (s, 3H).

### Step 2: Preparation of 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 6-aminohexanoate hydrochloride

The title compound was prepared in analogy to example 24, step 2 by using 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 6-((tert-butoxycarbonyl)amino)hexanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.34 g, yield: 94.4%). MS (m/z): [M+H]⁺ calcd for C₂₇H₃₉NO₆, 474.28; found, 474.2.

### Step 3: Preparation of Conjugate of HA and 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 6-aminohexanoate

The title compound was prepared in analogy to example 34, step 3 by using 2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl 6-aminohexanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.236 g, yield: 78.6%, DSR=5%). ¹H NMR (400 MHz, D₂O) δ 7.66-7.53 (m, 0.05H), 6.47-6.32 (m, 0.05H), 6.23-6.11 (m, 0.05H), 5.19-4.88 (m, 0.1H), 4.69-4.32 (m, 2.05H), 4.31-3.08 (m, 10.1H), 2.75-2.18 (m, 0.25H), 2.17-1.08 (m, 3.85H), 1.03-0.85 (m, 0.25H).

### Example 82

### Preparation of Conjugate of HA and 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 6-aminohexanoate

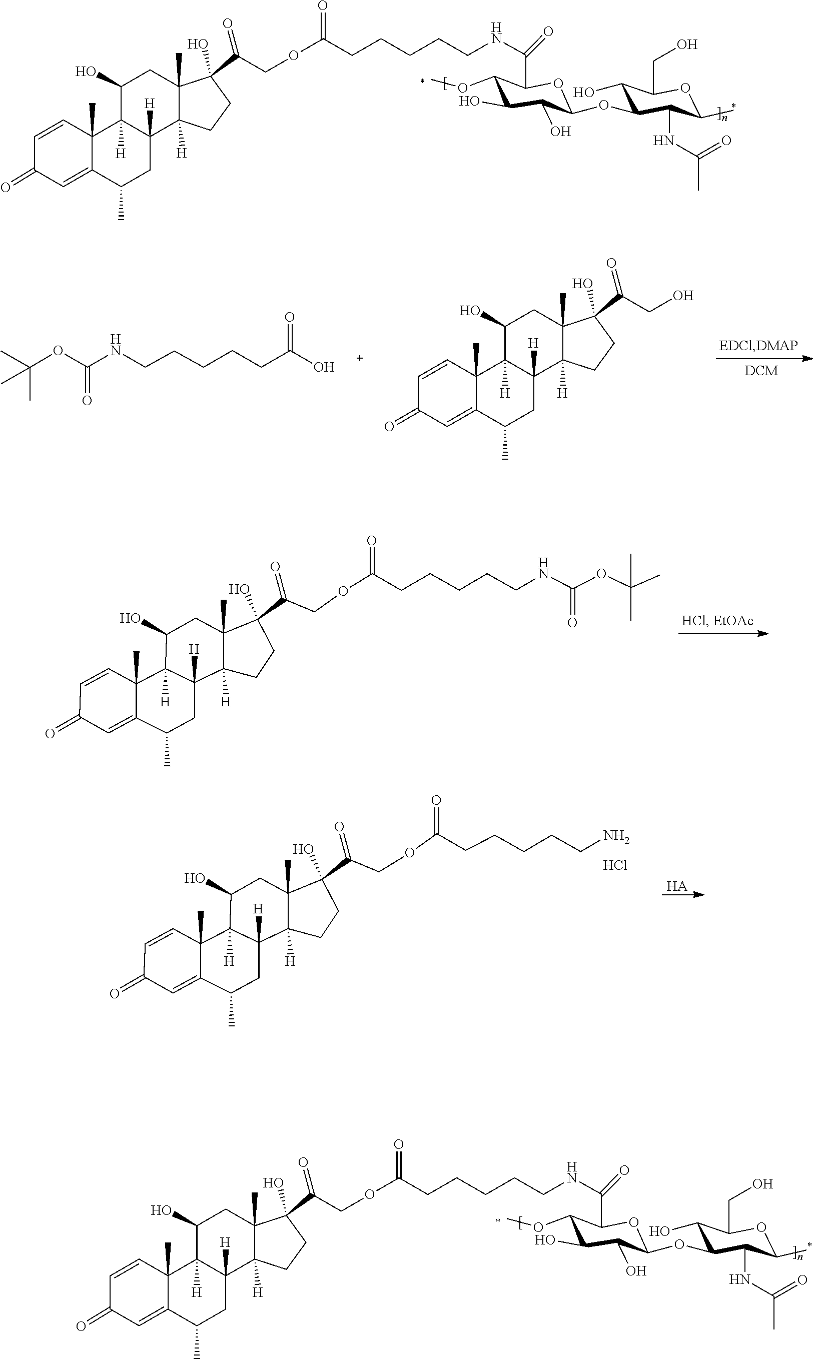

### Step 1: Preparation of 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 6-((tert-butoxycarbonyl)amino)hexanoate

The title compound was prepared in analogy to example 23, step 1 by using (6S,8S,9S,10R,11S,13S,14S,17R)-11,17-dihydroxy-17-(2-hydroxyacetyl)-6,10,13-trimethyl-6,7,8,9,10,11,12,13,14,15,16,17-dodecahydro-3H-cyclopenta[a]phenanthren-3-one instead of (6α,9α,11β,16β)-6,9-difluoro-11,21-dihydroxy-16,17-[(1-methylethylidene)bis(oxy)]pregna-1,4-diene-3,20-dione was obtained as a white solid (0.38 g, yield: 80.8%). MS (m/z): [M+H]⁺ calcd for C₃₃H₄₉NO₈, 588.35; found, 488.3 (M+H−100). ¹H NMR (400 MHz, DMSO) δ 7.33 (d, J=10.1 Hz, 1H), 6.76 (t, J=5.3 Hz, 1H), 6.18 (dd, J=10.1, 1.6 Hz, 1H), 5.83 (s, 1H), 5.39 (s, 1H), 5.06 (d, J=17.6 Hz, 1H), 4.74 (dd, J=14.2, 10.7 Hz, 2H), 4.29 (s, 1H), 2.90 (dd, J=12.9, 6.6 Hz, 2H), 2.72-2.60 (m, 1H), 2.51-2.44 (m, 1H), 2.37 (t, J=7.3 Hz, 2H), 2.08 (ddt, J=12.2, 8.8, 4.0 Hz, 2H), 1.87 (dt, J=16.7, 8.5 Hz, 1H), 1.74-1.51 (m, 5H), 1.50-1.23 (m, 18H), 1.05 (d, J=6.3 Hz, 3H), 0.91-0.84 (m, 1H), 0.83-0.76 (m, 3H), 0.68 (dd, J=24.9, 12.7 Hz, 1H).

### Step 2: Preparation of 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 6-aminohexanoate hydrochloride

The title compound was prepared in analogy to example 24, step 2 by using 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 6-((tert-butoxycarbonyl)amino)hexanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.27 g, yield: 85.7%). MS (m/z): [M+H]⁺ calcd for C₂₈H₄₁NO₆, 488.29; found, 488.2.

### Step 3: Preparation of Conjugate of HA and 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 6-aminohexanoate

The title compound was prepared in analogy to example 34, step 3 by using 2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl 6-aminohexanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.096 g, yield: 32%, DSR=12%). ¹H NMR (400 MHz, D₂O) δ 7.62-7.51 (m, 0.12H), 6.45-6.36 (m, 0.12H), 6.12 (s, 0.12H), 5.22-4.94 (m, 0.24H), 4.71-4.38 (m, 2.12H), 4.21-3.02 (m, 10.24H), 2.94-2.35 (m, 0.48H), 2.34-1.37 (m, 5.04H), 1.25-1.09 (m, 0.36), 1.06-0.81 (m, 0.6H).

### Example 83

### Preparation of Conjugate of HA and 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 6-aminohexanoate

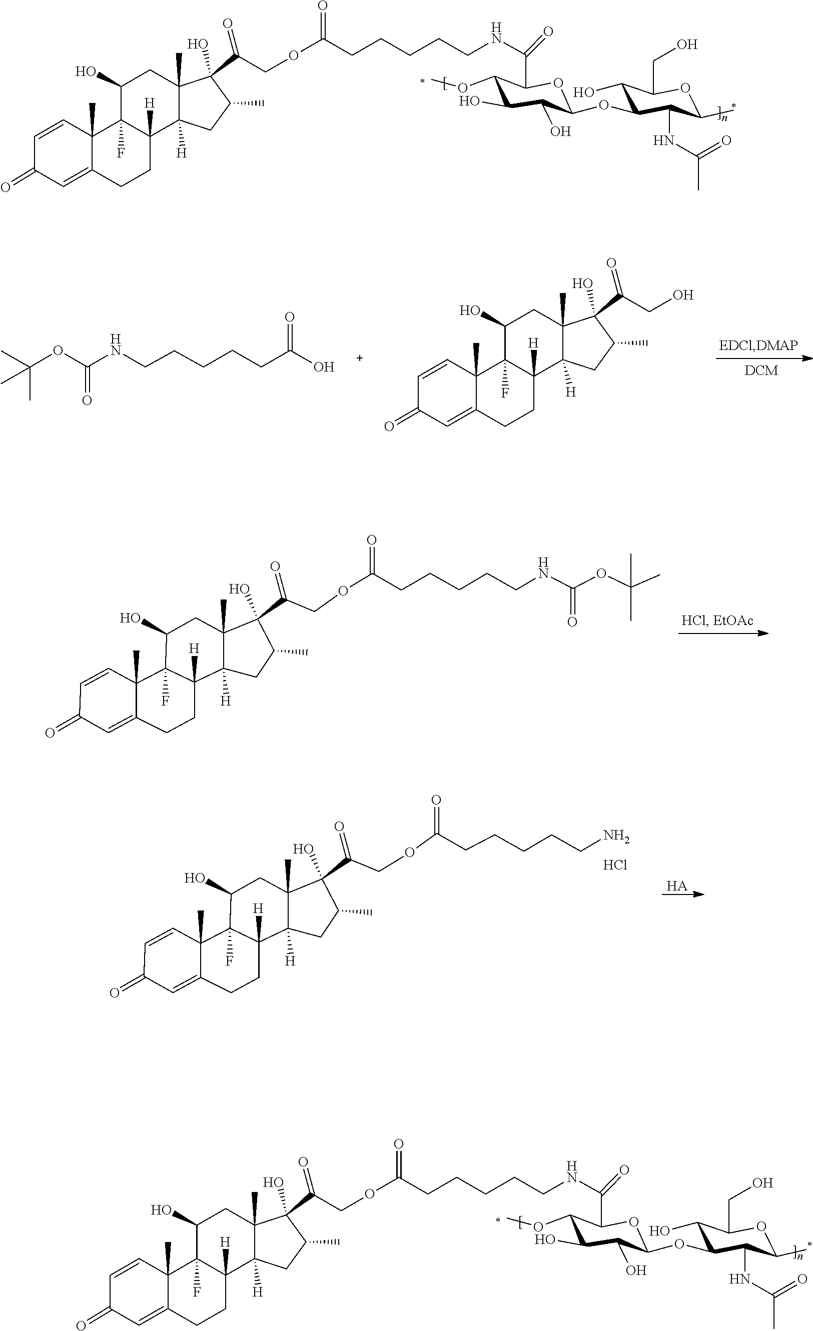

### Step 1: Preparation of 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 6-((tert-butoxycarbonyl)amino)hexanoate

The title compound was prepared in analogy to example 23, step 1 by using (8S,9R,10S,11S,13S,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one instead of (6α,9α,11β,16β)-6,9-difluoro-11,21-dihydroxy-16,17-[(1-methylethylidene)bis(oxy)]pregna-1,4-diene-3,20-dione was obtained as a white solid (0.35 g, yield: 75.5%). MS (m/z): [M+H]⁺ calcd for C₃₃H₄₈FNO₈, 606.34; found, 506.3 (M+H−100). ¹H NMR (400 MHz, DMSO) δ 7.29 (d, J=10.1 Hz, 1H), 6.75 (t, J=5.3 Hz, 1H), 6.22 (dd, J=10.1, 1.7 Hz, 1H), 6.01 (s, 1H), 5.42-5.23 (m, 2H), 4.97 (d, J=17.7 Hz, 1H), 4.82 (d, J=17.7 Hz, 1H), 4.21-4.10 (m, 1H), 2.90 (dd, J=12.9, 6.6 Hz, 2H), 2.71-2.54 (m, 1H), 2.48-2.25 (m, 4H), 2.18-2.04 (m, 2H), 1.98-1.76 (m, 2H), 1.64-1.21 (m, 21H), 1.12-0.98 (m, 4H), 0.97-0.77 (m, 3H).

### Step 2: Preparation of 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 6-aminohexanoate hydrochloride

The title compound was prepared in analogy to example 24, step 2 by using 2-((8S,9R,10S,11S,135,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 6-((tert-butoxycarbonyl)amino)hexanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.24 g, yield: 85.6%). MS (m/z): [M+H]⁺ calcd for C₂₈H₄₀FNO₆, 506.28; found, 506.2. ¹H NMR (400 MHz, D₂O) δ 7.45 (d, J=10.1 Hz, 1H), 6.35 (dd, J=10.1, 1.8 Hz, 1H), 6.15 (s, 1H), 5.07-4.87 (m, 2H), 4.31 (d, J=9.3 Hz, 1H), 3.00-2.86 (m, 2H), 2.75-2.62 (m, 1H), 2.58-2.33 (m, 4H), 2.18-2.07 (m, 2H), 1.93 (dd, J=20.8, 11.5 Hz, 3H), 1.69-1.54 (m, 5H), 1.53-1.34 (m, 6H), 1.19-1.09 (m, 1H), 1.02 (d, J=7.3 Hz, 3H), 0.89 (d, J=23.5 Hz, 3H).

### Step 3: Preparation of Conjugate of HA and 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 6-aminohexanoate

The title compound was prepared in analogy to example 34, step 3 by using 2-((8S,9R,105,115,135,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 6-aminohexanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.223 g, yield: 74.3%, DSR=8%). ¹H NMR (400 MHz, D₂O) δ 7.62-7.51 (m, 0.08H), 6.49-6.40 (m, 0.08H), 6.28 (s, 0.08H), 5.18-4.92 (m, 0.16H), 4.71-4.30 (m, 2.08H), 4.29-2.88 (m, 10.16H), 2.86-2.31 (m, 0.4H), 2.30-1.21 (m, 4.36H), 1.13 (d, J=6.9 Hz, 0.24H), 1.04 (s, 0.24H).

### Example 84

### Preparation of Conjugate of HA and 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 6-aminohexanoate

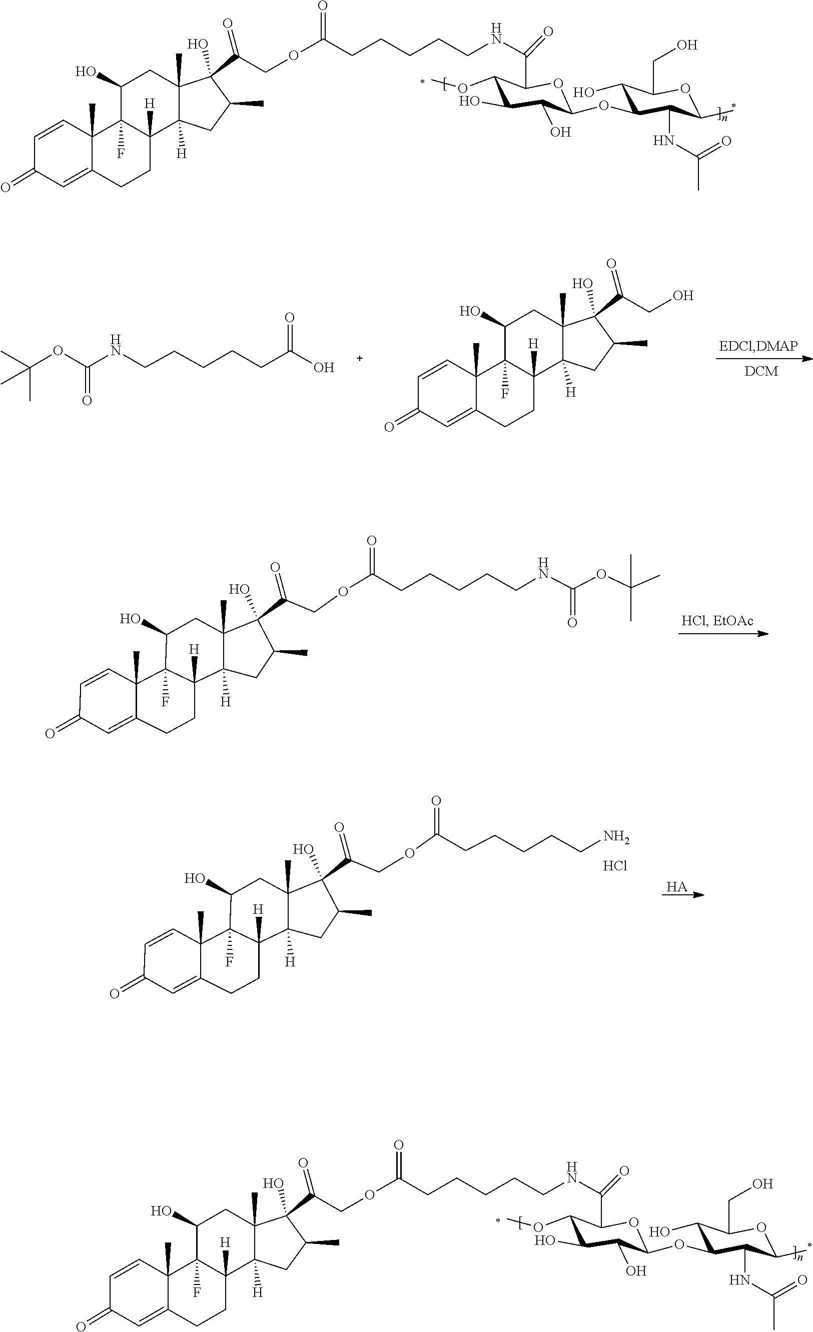

### Step 1: Preparation of 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 6-((tert-butoxycarbonyl)amino)hexanoate

The title compound was prepared in analogy to example 23, step 1 by using (8S,9R,10S,11S,13S,14S,16S,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one instead of (6α,9α,11β,16β)-6,9-difluoro-11,21-dihydroxy-16,17-[(1-methylethylidene)bis(oxy)]pregna-1,4-diene-3,20-dione was obtained as a white solid (0.42 g, yield: 90.7%). MS (m/z): [M+H]⁺ calcd for C₃₃H₄₈FNO₈, 606.34; found, 506.3 (M+H−100). ¹H NMR (400 MHz, DMSO) δ 7.30 (d, J=10.1 Hz, 1H), 6.76 (t, J=5.3 Hz, 1H), 6.23 (dd, J=10.1, 1.8 Hz, 1H), 6.01 (s, 1H), 5.40 (d, J=4.2 Hz, 1H), 5.15 (s, 1H), 5.02 (d, J=17.6 Hz, 1H), 4.80 (d, J=17.6 Hz, 1H), 4.19-4.11 (m, 1H), 2.97-2.83 (m, 3H), 2.61 (tt, J=20.7, 10.5 Hz, 1H), 2.44-2.27 (m, 4H), 2.24-2.07 (m, 2H), 1.82-1.72 (m, 1H), 1.71-1.45 (m, 7H), 1.44-1.23 (m, 14H), 1.10-1.04 (m, 1H), 0.94-0.82 (m, 3H), 0.81-0.73 (m, 3H).

### Step 2: Preparation of 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 6-aminohexanoate hydrochloride

The title compound was prepared in analogy to example 24, step 2 by using 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 6-((tert-butoxycarbonyl)amino)hexanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.3 g, yield: 85.7%). MS (m/z): [M+H]⁺ calcd for C₂₈H₄₀FNO₆, 506.28; found, 506.2. ¹H NMR (400 MHz, D₂O) δ 7.53 (d, J=10.0 Hz, 1H), 6.42 (d, J=9.9 Hz, 1H), 6.19 (d, J=14.1 Hz, 1H), 5.04 (t, J=11.5 Hz, 2H), 4.36 (t, J=17.3 Hz, 1H), 3.03 (t, J=7.4 Hz, 3H), 2.66 (d, J=27.1 Hz, 1H), 2.62-2.36 (m, 4H), 2.35-2.05 (m, 2H), 1.88 (d, J=32.2 Hz, 1H), 1.84-1.61 (m, 6H), 1.60-1.37 (m, 6H), 1.23 (m, 1H), 1.12-0.74 (m, 6H).

### Step 3: Preparation of Conjugate of HA and 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 6-aminohexanoate

The title compound was prepared in analogy to example 34, step 3 by using 2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 6-aminohexanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.196 g, yield: 65.3%, DSR=5%). ¹H NMR (400 MHz, D₂O) δ 7.60-7.53 (m, 0.05H), 6.51-6.44 (m, 0.05H), 6.27 (s, 0.05H), 5.12-4.89 (m, 0.1H), 4.69-4.28 (m, 2.05H), 4.27-2.99 (m, 10.15H), 2.98-2.34 (m, 0.25H), 2.33-1.27 (m, 3.8H), 1.02 (s, 0.15H), 0.93 (s, 0.15H).

### Example 85

### Preparation of Conjugate of HA and 5-aminopentyl (2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl) carbonate

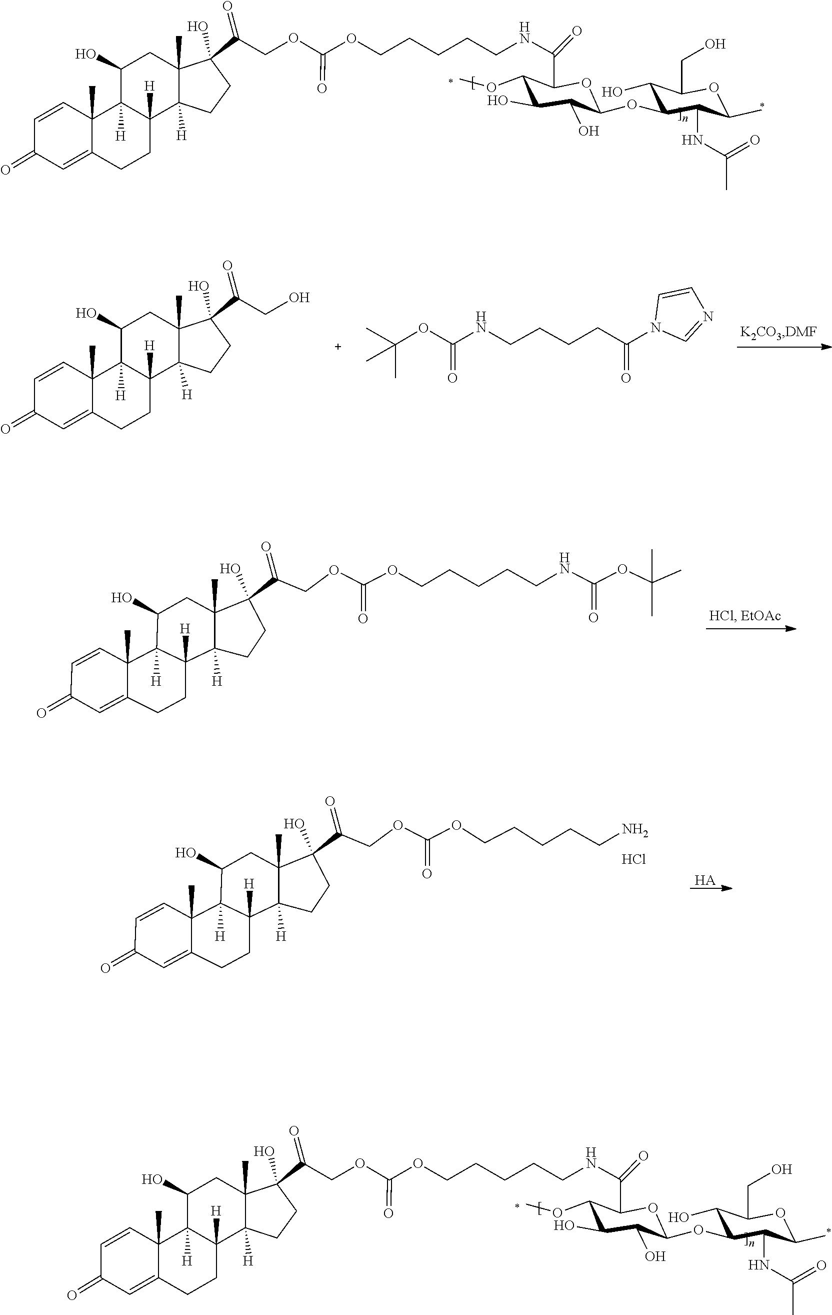

### Step 1: Preparation of tert-butyl (5-(((2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethoxy)carbonyl)oxy)pentyl)carbamate

The title compound was prepared in analogy to example 62, step 1 by using 5-((tert-butoxycarbonyl)amino)pentyl 1H-imidazole-1-carboxylate instead of 4-((tert-butoxycarbonyl)amino)butyl 1H-imidazole-1-carboxylate was obtained as a white solid (0.227 g, yield: 48.5%). MS (m/z): [M+H]⁺ calcd for C₃₂H₄₇NO₉, 590.33; found, 612.3 [M+Na]⁺.

### Step 2: Preparation of 5-aminopentyl (2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl) carbonate hydrochloride

The title compound was prepared in analogy to example 24, step 2 by using tert-butyl (5-(((2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethoxy)carbonyl)oxy)pentyl)carbamate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.17 g, yield: 90%). MS (m/z): [M+H]⁺ calcd for C₂₇H₃₉NO₇, 490.27; found, 490.2. ¹H NMR (400 MHz, D₂O) δ 7.56 (d, J=10.0 Hz, 1H), 6.35 (d, J=9.8 Hz, 1H), 6.13 (s, 1H), 5.16 (d, J=18.1 Hz, 1H), 4.99 (d, J=18.2 Hz, 1H), 4.51 (s, 1H), 4.25 (s, 2H), 3.14-2.94 (m, 2H), 2.72-2.51 (m, 2H), 2.42 (d, J=9.7 Hz, 1H), 2.14 (t, J=19.6 Hz, 2H), 1.94 (d, J=11.7 Hz, 1H), 1.89-1.06 (m, 16H), 0.87 (s, 3H).

### Step 3: Preparation of Conjugate of HA and 5-aminopentyl (2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl) carbonate

The title compound was prepared in analogy to example 34, step 3 by using 5-aminopentyl (2-((8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.333 g, yield: 73.6%, DSR=23%). ¹H NMR (400 MHz, D₂O) δ 7.74-7.56 (m, 0.23H), 6.54-6.38 (m, 0.23H), 6.19 (s, 0.23H), 5.28-4.96 (m, 0.46H), 4.75-4.18 (m, 2.69H), 4.16-2.92 (m, 10.46H), 2.91-1.06 (m, 8.06H), 0.94 (s, 0.69H).

### Example 86

### Preparation of Conjugate of HA and 5-aminopentyl (2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl) carbonate

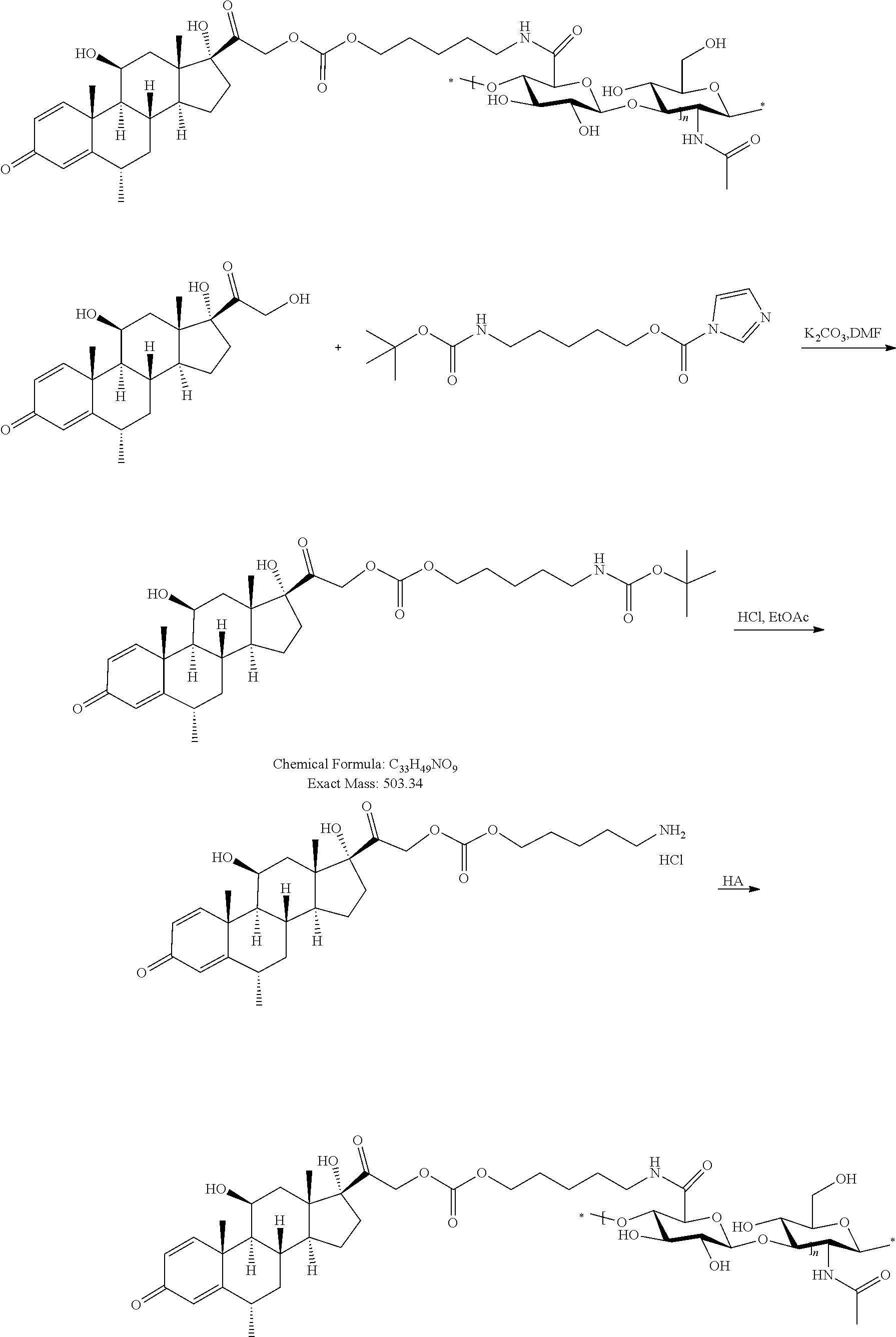

### Step 1: Preparation of tert-butyl (5-(((2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethoxy)carbonyl)oxy)pentyl)carbamate

The title compound was prepared in analogy to example 68, step 1 by using 5-((tert-butoxycarbonyl)amino)pentyl 1H-imidazole-1-carboxylate instead of 4-((tert-butoxycarbonyl)amino)butyl 1H-imidazole-1-carboxylate was obtained as a white solid (0.109 g, yield: 22.4%). MS (m/z): [M+H]⁺ calcd for C₃₃H₄₉NO₉, 604.34; found, 626.4 [M+Na]⁺.

### Step 2: Preparation of 5-aminopentyl (2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl) carbonate hydrochloride

The title compound was prepared in analogy to example 24, step 2 by using tert-butyl (5-(((2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethoxy)carbonyl)oxy)pentyl)carbamate instead of 2-((2S,6αS,6βR,7S,8αaS,83S,11αR,12αS,12βS)-2,6-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.09 g, yield: 99%). MS (m/z): [M+H]⁺ calcd for C₂₈H₄₁NO₇, 504.29; found, 504.2. ¹H NMR (400 MHz, D₂O) δ 7.60 (d, J=10.0 Hz, 1H), 6.40 (dd, J=10.0, 1.7 Hz, 1H), 6.13 (s, 1H), 5.19 (d, J=18.1 Hz, 1H), 5.02 (d, J=18.1 Hz, 1H), 4.53 (d, J=2.5 Hz, 1H), 4.37-4.22 (m, 2H), 3.06 (dd, J=9.3, 5.7 Hz, 2H), 2.82-2.73 (m, 1H), 2.64 (t, J=13.3 Hz, 1H), 2.33-2.17 (m, 2H), 1.96 (d, J=10.7 Hz, 1H), 1.89-1.39 (m, 14H), 1.24-1.03 (m, 4H), 0.96-0.80 (m, 4H).

### Step 3: Preparation of Conjugate of HA and 5-aminopentyl (2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl) carbonate

The title compound was prepared in analogy to example 34, step 3 by using 5-aminopentyl (2-((6S,8S,9S,10R,11S,13S,14S,17R)-11,17-dihydroxy-6,10,13-trimethyl-3-oxo-6,7,8,9,10,11,12,13,14,15,16,17-dodecahydro-3H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.144 g, yield: 73.8%, DSR=27%). ¹FH NMR (400 MHz, D₂O) δ 7.72-7.54 (m, 0.27H), 6.49-6.32 (m, 0.27H), 6.17 (s, 0.27H), 5.30-4.92 (m, 0.54H), 4.78-4.19 (m, 2.81H), 4.18-3.97 (m, 10.54H), 3.96-1.31 (m, 8.13H), 1.30-1.04 (m, 1.08H), 1.03-0.77 (m, 1.08H).

### Example 87

### Preparation of Conjugate of HA and 5-aminopentyl (2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate

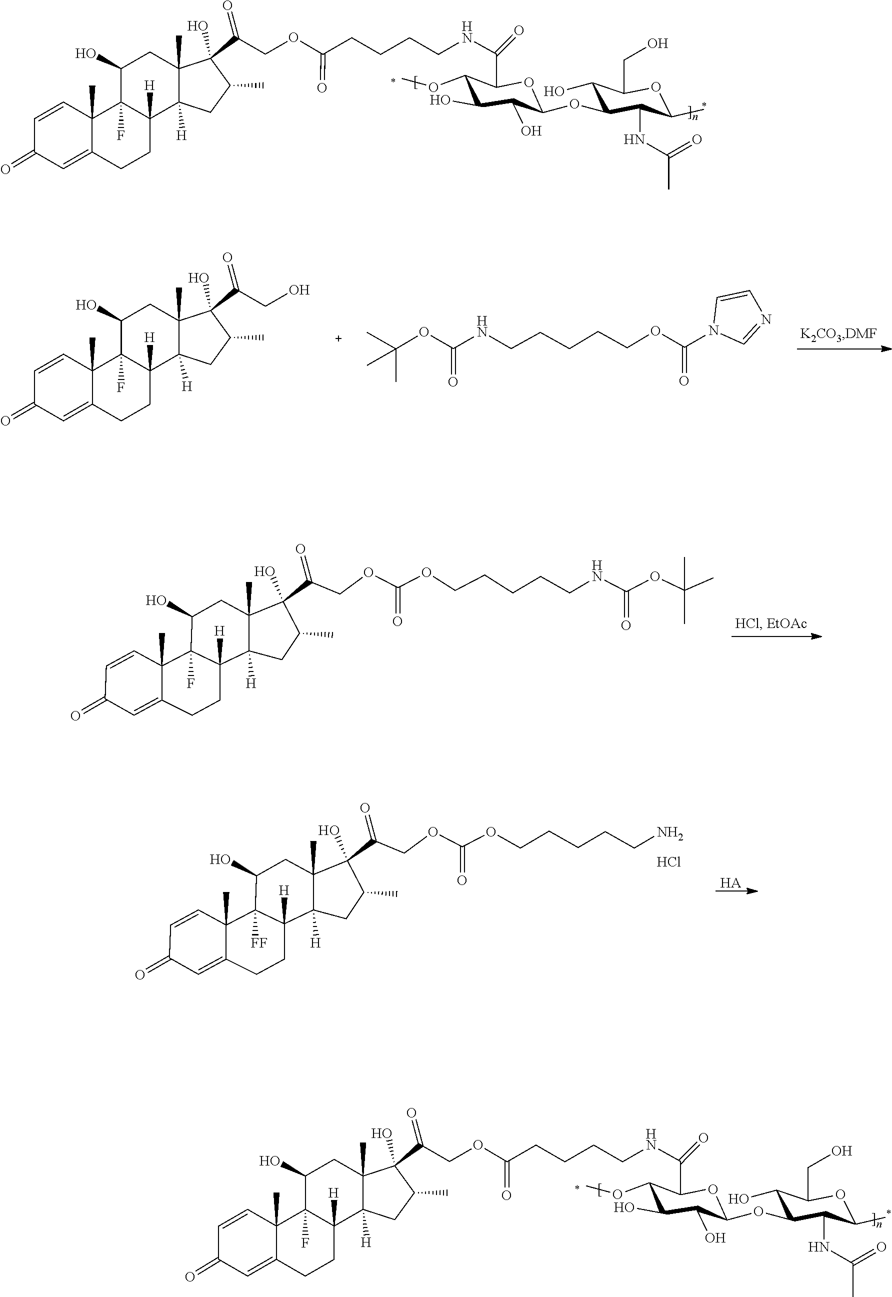

### Step 1: Preparation of tert-butyl (5-(((2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)carbonyl)oxy)pentyl)carbamate

The title compound was prepared in analogy to example 70, step 1 by using 5-((tert-butoxycarbonyl)amino)pentyl 1H-imidazole-1-carboxylate instead of 4-((tert-butoxycarbonyl)amino)butyl 1H-imidazole-1-carboxylate was obtained as a white solid (0.242 g, yield: 47.4%). MS (m/z): [M+H]⁺ calcd for C₃₃H₄₈FNO₉, 622.33; found, 644.3 [M+Na]⁺. ¹H NMR (400 MHz, D₂O) δ 7.28 (d, J=10.1 Hz, 1H), 6.75 (d, J=5.3 Hz, 1H), 6.21 (dd, J=10.1, 1.8 Hz, 1H), 6.00 (s, 1H), 5.34 (s, 1H), 5.29 (d, J=3.6 Hz, 1H), 4.97 (d, J=17.8 Hz, 1H), 4.79 (d, J=17.8 Hz, 1H), 4.18-4.00 (m, 3H), 3.39-3.24 (m, 2H), 2.62 (td, J=13.4, 5.9 Hz, 1H), 2.47-2.28 (m, 2H), 2.16-2.02 (m, 2H), 2.01-1.76 (m, 3H), 1.67-1.54 (m, 2H), 1.50 (d, J=13.8 Hz, 3H), 1.45-1.24 (m, 15H), 1.11-0.97 (m, 4H), 0.92 (s, 3H).

### Step 2: Preparation of 5-aminopentyl (2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate hydrochloride

The title compound was prepared in analogy to example 24, step 2 by using tert-butyl (5-(((2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)carbonyl)oxy)pentyl)carbamate instead of 2-((2S,6αS,6βR,7S,8αaS,83S,11αR,12αS,12βS)-2,6-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.2 g, yield: 98%). MS (m/z): [M+H]⁺ calcd for C₂₈H₄₀FNO₇, 522.28; found, 522.3. ¹H NMR (400 MHz, D₂O) δ 7.54 (d, J=10.0 Hz, 1H), 6.43 (d, J=10.1 Hz, 1H), 6.22 (s, 1H), 5.07 (q, J=18.3 Hz, 2H), 4.41 (t, J=15.5 Hz, 1H), 4.31-4.19 (m, 2H), 3.05 (d, J=9.7 Hz, 2H), 2.73 (dd, J=10.8, 5.7 Hz, 1H), 2.66-2.39 (m, 2H), 2.20 (t, J=11.1 Hz, 2H), 2.04 (d, J=10.0 Hz, 3H), 1.90-1.67 (m, 4H), 1.66-1.40 (m, 7H), 1.36-1.17 (m, 1H), 1.16-1.06 (m, 3H), 0.97 (d, J=46.3 Hz, 3H).

### Step 3: Preparation of Conjugate of HA and 5-aminopentyl (2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate

The title compound was prepared in analogy to example 34, step 3 by using 5-aminopentyl (2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.277 g, yield: 68.5%, DSR=12%). ¹H NMR (400 MHz, D₂O) δ 7.59 (d, J=10.1 Hz, 0.12H), 6.48 (d, J=10.2 Hz, 0.12H), 6.29 (s, 0.12H), 5.12 (q, J=18.4 Hz, 0.24H), 4.73-4.22 (m, 2.36H), 4.21-3.01 (m, 10.24H), 3.00-2.49 (m, 0.36H), 2.35-1.90 (m, 3.6H), 1.86-1.41 (m, 1.32H), 1.36-0.93 (m, 0.84H).

### Example 88

### Preparation of Conjugate of HA and 5-aminopentyl (2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate

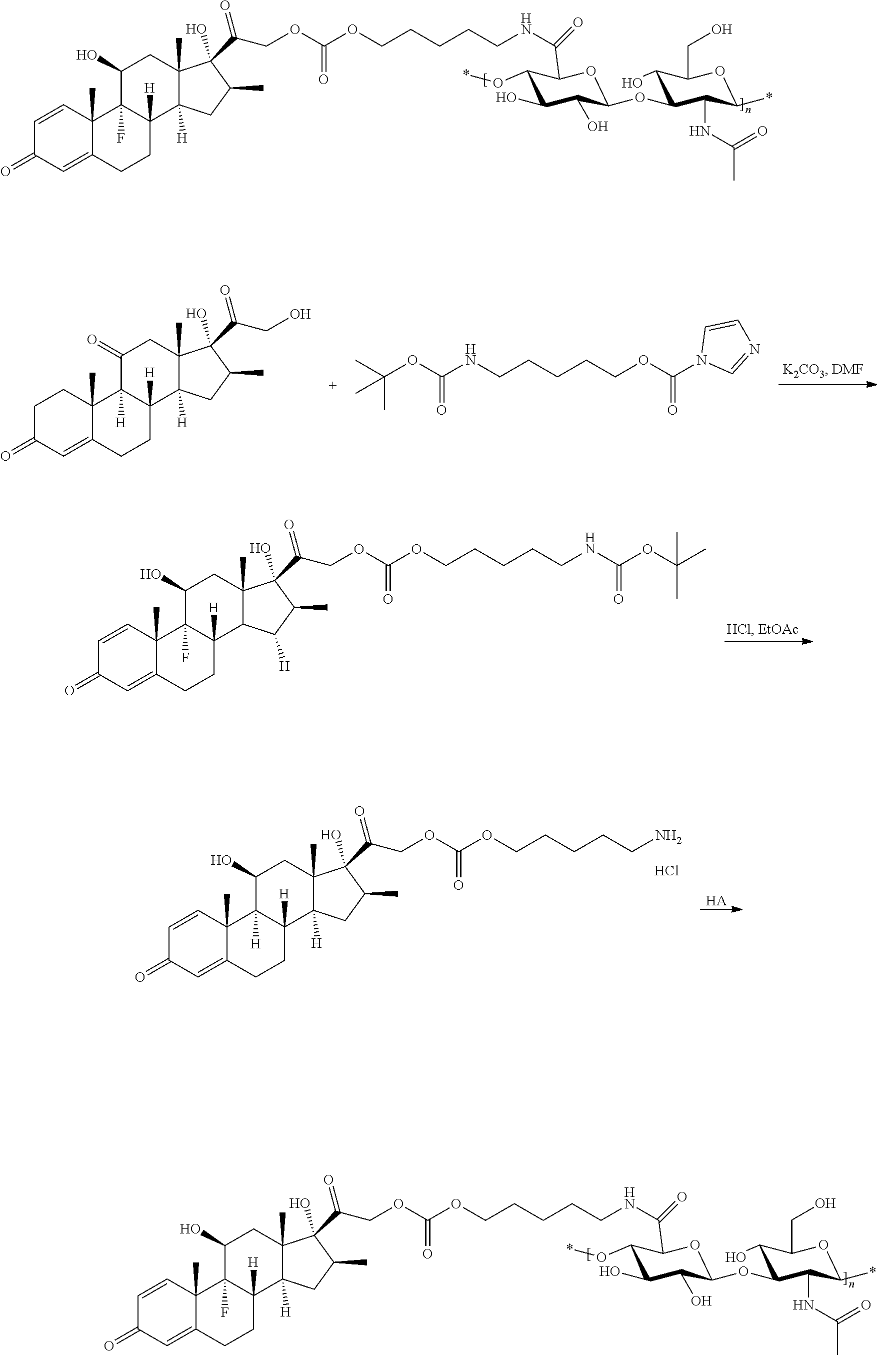

### Step 1: Preparation of tert-butyl (5-(((2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)carbonyl)oxy)pentyl)carbamate

The title compound was prepared in analogy to example 72, step 1 by using 5-((tert-butoxycarbonyl)amino)pentyl 1H-imidazole-1-carboxylate instead of 4-((tert-butoxycarbonyl)amino)butyl 1H-imidazole-1-carboxylate was obtained as a white solid (0.149 g, yield: 29.2%). MS (m/z): [M+H]⁺ calcd for C₃₃H₄₈FNO₉, 622.33; found, 644.3 [M+Na]⁺. ¹H NMR (400 MHz, DMSO) δ 7.30 (d, J=10.1 Hz, 1H), 6.81-6.69 (m, 1H), 6.23 (dd, J=10.1, 1.7 Hz, 1H), 6.01 (s, 1H), 5.41 (d, J=4.4 Hz, 1H), 5.18 (s, 1H), 5.07 (d, J=17.8 Hz, 1H), 4.77 (d, J=17.7 Hz, 1H), 4.12 (dt, J=13.0, 5.8 Hz, 3H), 3.42-3.33 (m, 2H), 2.70-2.56 (m, 1H), 2.44-2.27 (m, 2H), 2.22-2.06 (m, 2H), 1.84-1.73 (m, 1H), 1.72-1.20 (m, 22H), 1.08 (ddd, J=11.7, 7.7, 3.7 Hz, 1H), 0.90 (s, 3H), 0.80 (d, J=7.2 Hz, 3H).

### Step 2: Preparation of 5-aminopentyl (2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate hydrochloride

The title compound was prepared in analogy to example 24, step 2 by using tert-butyl (5-(((2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)carbonyl)oxy)pentyl)carbamate instead of 2-((2S,6αS,6βR,7S,8αaS,83S,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.12 g, yield: 96%). MS (m/z): [M+H]⁺ calcd for C₂₈H₄₀FNO₇, 522.28; found, 522.3. ¹H NMR (400 MHz, D2O) δ 7.90 (s, 4H), 7.33 (d, J=10.1 Hz, 1H), 6.21 (dd, J=10.1, 1.8 Hz, 1H), 5.99 (s, 1H), 5.48 (d, J=4.8 Hz, 1H), 5.19 (s, 1H), 5.08 (d, J=17.8 Hz, 1H), 4.77 (d, J=17.7 Hz, 1H), 4.19-4.05 (m, 3H), 2.87 (ddd, J=11.1, 6.6, 4.1 Hz, 1H), 2.83-2.70 (m, 1H), 2.62 (ddd, J=18.7, 16.3, 9.8 Hz, 1H), 2.44-2.26 (m, 2H), 2.24-2.03 (m, 2H), 1.74 (dd, J=22.4, 16.4 Hz, 1H), 1.68-1.20 (m, 13H), 1.06 (ddd, J=11.9, 8.1, 3.9 Hz, 1H), 0.98-0.83 (m, 3H), 0.78 (d, J=7.2 Hz, 3H).

### Step 3: Preparation of Conjugate of HA and 5-aminopentyl (2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate

The title compound was prepared in analogy to example 34, step 3 by using 5-aminopentyl (2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.223 g, yield: 74.3%, DSR=22%). ¹FH NMR (400 MHz, D₂O) δ 7.66-7.54 (m, 0.22H), 6.56-6.44 (m, 0.22H), 6.28 (s, 0.22H), 5.07 (d, J=84.3 Hz, 0.44H), 4.79-4.20 (m, 2.66H), 4.19-2.84 (m, 10.44H), 2.83-1.86 (m, 4.76H), 1.83-1.14 (m, 2.42H), 1.09-0.79 (m, 1.54H).

### Example 89

### Preparation of Conjugate of HA and 5-aminopentyl (2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl) carbonate

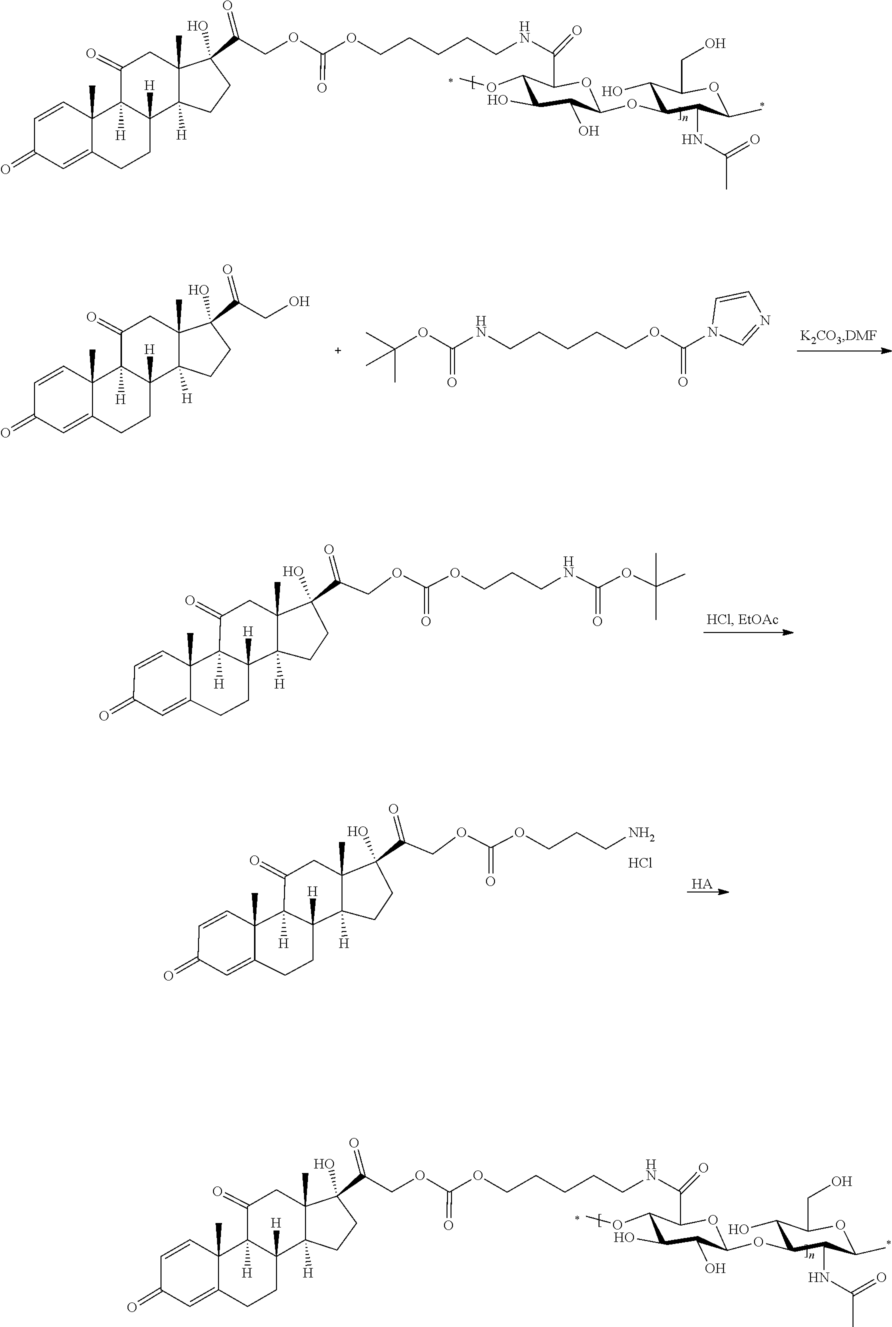

### Step 1: Preparation of tert-butyl (5-(((2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethoxy)carbonyl)oxy)pentyl)carbamate

The title compound was prepared in analogy to example 60, step 1 by using 5-((tert-butoxycarbonyl)amino)pentyl 1H-imidazole-1-carboxylate instead of 4-((tert-butoxycarbonyl)amino)butyl 1H-imidazole-1-carboxylate was obtained as a white solid (0.126 g, yield: 27%). MS (m/z): [M+H]⁺ calcd for C₃₂H₄₅NO₉, 588.31; found, 610.2 [M+Na]⁺.

### Step 2: Preparation of 5-aminopentyl (2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl) carbonate hydrochloride

The title compound was prepared in analogy to example 24, step 2 by using tert-butyl (5-(((2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethoxy)carbonyl)oxy)pentyl)carbamate instead of 2-((2S,6αS,6βR,7S,8αaS,83S,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.1 g, yield: 95%). MS (m/z): [M+H]⁺ calcd for C₂₇H₃₇NO₇, 488.26; found, 488.3. ¹H NMR (400 MHz, D₂O) δ 7.89 (d, J=10.2 Hz, 1H), 6.33 (d, J=9.9 Hz, 1H), 6.25 (s, 1H), 5.07 (dt, J=37.9, 18.9 Hz, 2H), 4.29 (m, 2H), 3.06 (t, J=7.5 Hz, 2H), 2.95 (d, J=12.3 Hz, 1H), 2.78-2.57 (m, 1H), 2.53 (d, J=13.1 Hz, 1H), 2.49-2.15 (m, 5H), 1.97 (s, 1H), 1.90-1.69 (m, 4H), 1.68-1.12 (m, 9H), 0.71 (s, 3H).

### Step 3: Preparation of Conjugate of HA and 5-aminopentyl (2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl) carbonate

The title compound was prepared in analogy to example 34, step 3 by using 5-aminopentyl (2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.159 g, yield: 62.5%, DSR=24%). ¹H NMR (400 MHz, D₂O) δ 7.96-7.86 (m, 0.24H), 6.35 (d, J=8.0 Hz, 0.24H), 6.26 (s, 0.24H), 5.30-4.94 (m, 0.48H), 4.79-4.18 (m, 2.48H), 4.15-2.88 (m, 10.72H), 2.85-1.73 (m, 5.88H), 1.72-1.10 (m, 2.16H), 0.73 (s, 0.72H).

### Example 90

### Preparation of Conjugate of HA and 5-aminopentyl (2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl) carbonate

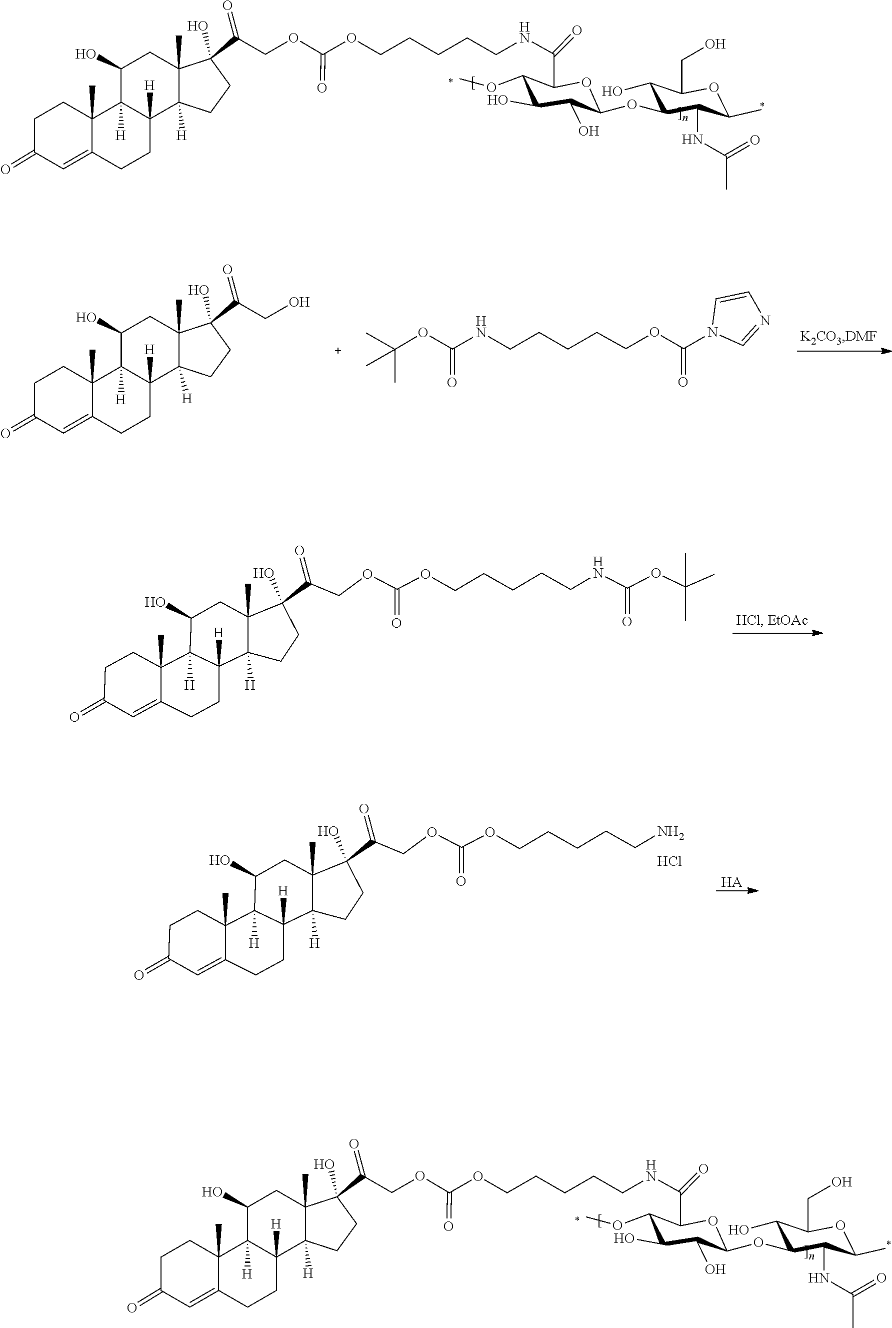

### Step 1: Preparation of tert-butyl (5-(((2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethoxy)carbonyl)oxy)pentyl)carbamate

The title compound was prepared in analogy to example 64, step 1 by using 5-((tert-butoxycarbonyl)amino)pentyl 1H-imidazole-1-carboxylate instead of 4-((tert-butoxycarbonyl)amino)butyl 1H-imidazole-1-carboxylate was obtained as a white solid (0.093 g, yield: 19.70%). MS (m/z): [M+H]⁺ calcd for C₃₂H₄₉NO₉, 592.34; found, 614.3 [M+Na]⁺.

### Step 2: Preparation of 5-aminopentyl (2-((8S,9S,1R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl) carbonate hydrochloride

The title compound was prepared in analogy to example 24, step 2 by using tert-butyl (5-(((2-((8 S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethoxy)carbonyl)oxy)pentyl)carbamate instead of 2-((2S,6αS,6βR,7S,8αaS,83S,11αR,12αS,12βS)-2,6-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.07 g, yield: 90%). MS (m/z): [M+H]⁺ calcd for C₂₇H₄₁NO₇, 492.29; found, 492.2. ¹H NMR (400 MHz, D₂O) δ 5.81 (s, 1H), 5.25-5.16 (m, 1H), 5.10-5.00 (m, 1H), 4.53 (d, J=2.8 Hz, 1H), 4.35-4.21 (m, 2H), 3.06 (t, J=7.5 Hz, 2H), 2.74-2.54 (m, 4H), 2.53-2.32 (m, 2H), 2.31-1.68 (m, 11H), 1.67-1.57 (m, 1H), 1.56-1.35 (m, 6H), 1.28-1.09 (m, 2H), 0.92 (s, 3H).

### Step 3: Preparation of Conjugate of HA and 5-aminopentyl (2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl) carbonate

The title compound was prepared in analogy to example 34, step 3 by using 5-aminopentyl (2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa, 0.116 g, yield: 76.3%, DSR=18%). ¹H NMR (400 MHz, D₂O) δ 5.83 (s, 0.18H), 5.39-4.97 (m, 0.36H), 4.73-4.17 (m, 2.54H), 4.15-3.03 (m, 10.36H), 3.01-1.29 (m, 7.32H), 1.28-1.07 (m, 0.36H), 0.91 (s, 0.54H).

### Example 91

### Preparation of Conjugate of HA and 5-aminopentyl (2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl) carbonate

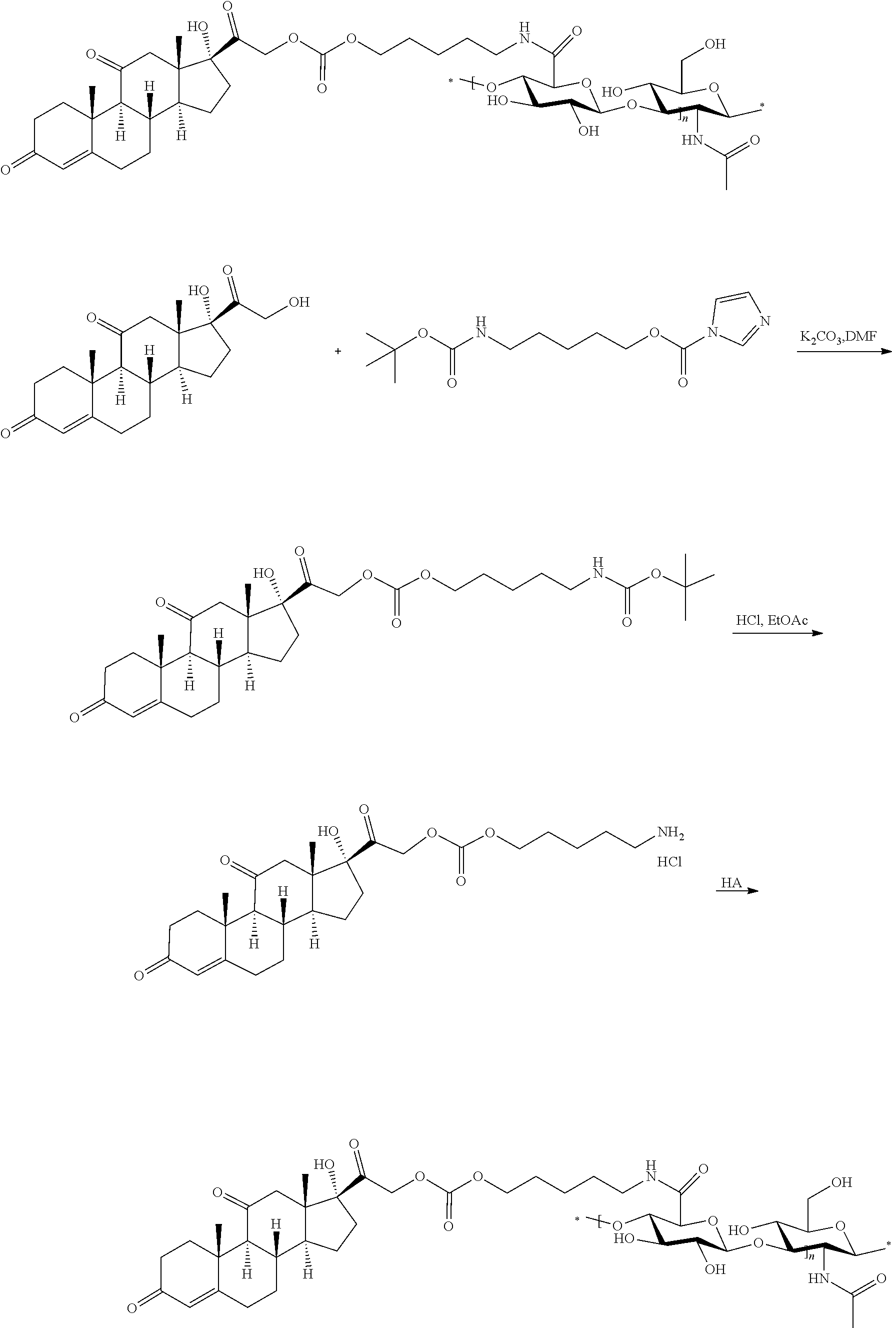

### Step 1: Preparation of tert-butyl (5-(((2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethoxy)carbonyl)oxy)pentyl)carbamate

The title compound was prepared in analogy to example 66, step 1 by using 5-((tert-butoxycarbonyl)amino)pentyl 1H-imidazole-1-carboxylate instead of 4-((tert-butoxycarbonyl)amino)butyl 1H-imidazole-1-carboxylate was obtained as a white solid (0.068 g, yield: 14.5%). MS (m/z): [M+H]⁺ calcd for C₃₂H₄₇NO₉, 590.33; found, 612.4 [M+Na]⁺.

### Step 2: Preparation of 5-aminopentyl (2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl) carbonate hydrochloride

The title compound was prepared in analogy to example 24, step 2 by using tert-butyl (5-(((2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethoxy)carbonyl)oxy)pentyl)carbamate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.05 g, yield: 88%). MS (m/z): [M+H]⁺ calcd for C₂₇H₃₉NO₇, 490.27; found, 490.3. ¹H NMR (400 MHz, D₂O) δ 5.86 (s, 1H), 5.19 (d, J=18.3 Hz, 1H), 5.02 (d, J=18.3 Hz, 1H), 4.28 (t, J=5.3 Hz, 2H), 3.06 (t, J=7.5 Hz, 2H), 2.98 (d, J=12.2 Hz, 1H), 2.77-2.50 (m, 4H), 2.49-2.25 (m, 5H), 2.24-1.98 (m, 2H), 1.97-1.69 (m, 6H), 1.66-1.34 (m, 8H), 0.68 (s, 3H).

### Step 3: Preparation of Conjugate of HA and 5-aminopentyl (2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl) carbonate

The title compound was prepared in analogy to example 34, step 3 by using 5-aminopentyl (2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl) carbonate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.092 g, yield: 71.8%, DSR=30%). ¹H NMR (400 MHz, D₂O) δ 5.88 (s, 0.3H), 5.25-4.84 (m, 0.6H), 4.73-4.18 (m, 2.6H), 4.16-3.04 (m, 10.9H), 2.99-0.85 (m, 10.5H), 0.68 (s, 0.9H).

### Example 92

### Preparation of Conjugate of HA and 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 6-aminohexanoate

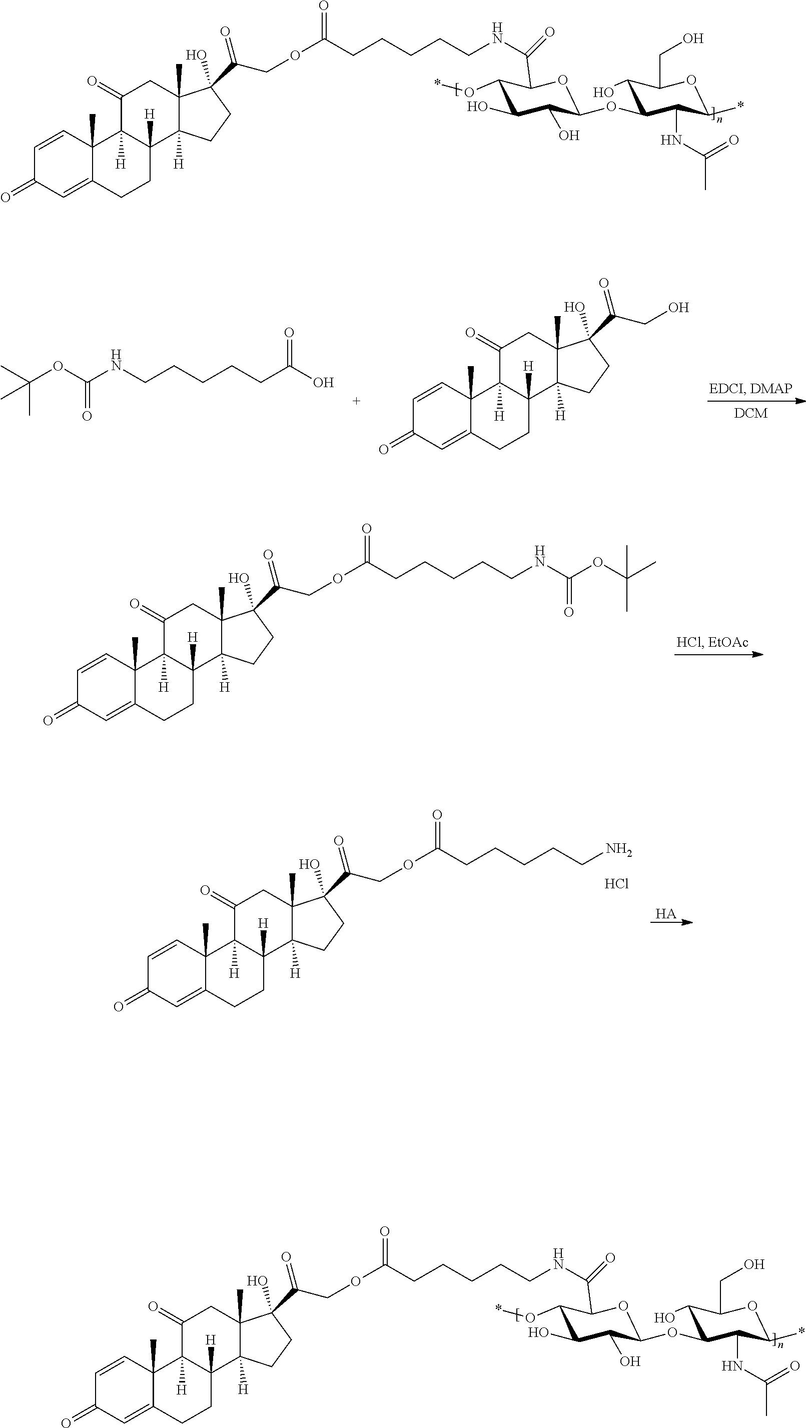

### Step 1: Preparation of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 6-((tert-butoxycarbonyl)amino)hexanoate

The title compound was prepared in analogy to example 23, step 1 by using (8S,9S,10R,13S,14S,17R)-17-hydroxy-17-(2-hydroxyacetyl)-10,13-dimethyl-7,8,9,10,12,13,14,15,16,17-decahydro-3H-cyclopenta[a]phenanthrene-3,11(6H)-dione instead of (6α,9α,11β,16β)-6,9-difluoro-11,21-dihydroxy-16,17-[(1-methylethylidene)bis(oxy)]pregna-1,4-diene-3,20-dione was obtained as a white solid (0.4 g, yield: 80.4%). MS (m/z): [M+H]⁺ calcd for C₃₂H₄₅NO₈, 572.31; found, 594.2 [M+Na]⁺. ¹H NMR (400 MHz, DMSO) δ 7.62 (d, J=10.2 Hz, 1H), 6.74 (s, 1H), 6.12 (dd, J=10.3, 1.9 Hz, 1H), 6.02 (s, 1H), 5.81 (s, 1H), 4.92 (d, J=17.7 Hz, 1H), 4.81 (d, J=17.7 Hz, 1H), 2.90 (dd, J=12.4, 5.9 Hz, 3H), 2.57-2.52 (m, 1H), 2.42-2.31 (m, 4H), 2.20 (dd, J=20.1, 8.5 Hz, 2H), 2.04 (ddd, J=17.7, 10.3, 4.4 Hz, 2H), 1.84-1.72 (m, 1H), 1.71-1.63 (m, 1H), 1.59-1.49 (m, 2H), 1.43-1.33 (m, 15H), 1.32-1.15 (m, 4H), 0.52 (s, 3H).

### Step 2: Preparation of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 6-aminohexanoate hydrochloride

The title compound was prepared in analogy to example 24, step 2 by using 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 6-((tert-butoxycarbonyl)amino)hexanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.31 g, yield: 93.9%). MS (m/z): [M+H]⁺ calcd for C₂₇H₃₇NO₆, 472.26; found, 472.2. ¹H NMR (400 MHz, D₂O) δ 7.89 (d, J=10.2 Hz, 1H), 6.33 (dd, J=10.2, 2.0 Hz, 1H), 6.24 (s, 1H), 5.12 (d, J=18.1 Hz, 1H), 5.02 (d, J=18.1 Hz, 1H), 3.06 (t, J=7.5 Hz, 2H), 2.95 (d, J=12.2 Hz, 1H), 2.76-2.50 (m, 5H), 2.48-2.34 (m, 2H), 2.33-2.16 (m, 3H), 1.98 (ddd, J=16.2, 10.8, 3.1 Hz, 1H), 1.89-1.66 (m, 5H), 1.65-1.43 (m, 6H), 1.42-1.27 (m, 1H), 0.69 (d, J=10.3 Hz, 3H).

### Step 3: Preparation of Conjugate of HA and 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 6-aminohexanoate

The title compound was prepared in analogy to example 34, step 3 by using 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 6-aminohexanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid (sodium hyaluronate MW 500 KDa 0.211 g, yield: 70.3%, DSR=14%). ¹H NMR (400 MHz, D₂O) δ 7.98-7.80 (m, 0.14H), 6.32 (s, 0.14H), 6.25 (s, 0.14H), 5.13-4.95 (m, 0.28H), 4.76-4.34 (m, 2H), 4.23-3.00 (m, 10.42H), 2.64-2.37 (m, 1.4H), 2.36-1.72 (m, 3.84H), 1.67-1.47 (m, 0.84H), 1.46-1.04 (m, 0.14H), 0.72 (s, 0.42H).

### Example 93

### Preparation of Conjugate of HA and 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 6-aminohexanoate

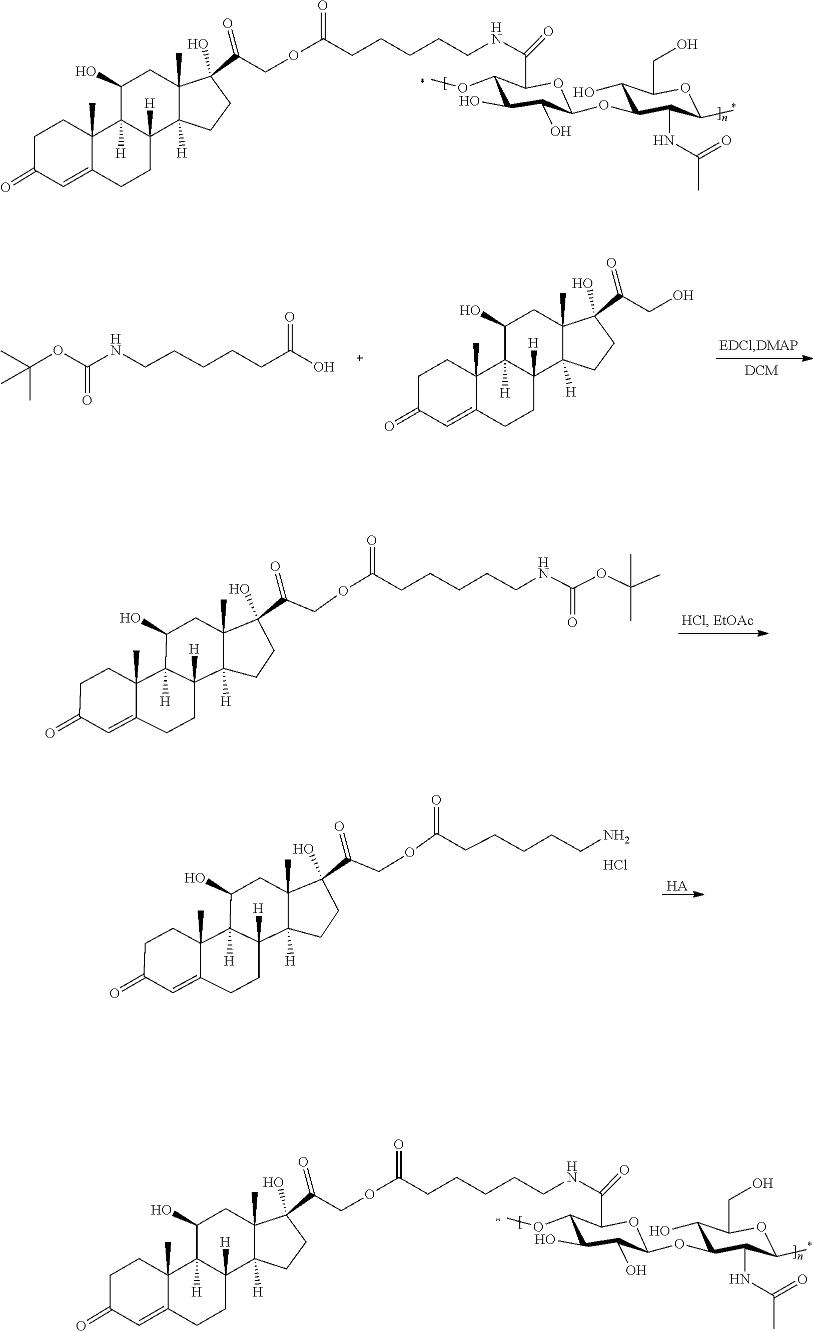

### Step 1: Preparation of 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 6-((tert-butoxycarbonyl)amino)hexanoate

The title compound was prepared in analogy to example 23, step 1 by using (8S,9S,10R,11S,13S,14S,17R)-11,17-dihydroxy-17-(2-hydroxyacetyl)-10,13-dimethyl-1,2,6,7,8,9,10,11,12,13,14,15,16,17-tetradecahydro-3H-cyclopenta[a]phenanthren-3-one instead of (6α,9α,11β,16β)-6,9-difluoro-11,21-dihydroxy-16,17-[(1-methylethylidene)bis(oxy)]pregna-1,4-diene-3,20-dione was obtained as a white solid (0.41 g, yield: 82.8%). MS (m/z): [M+H]⁺ calcd for C₃₂H₄₉NO₈, 576.35; found, 598.2 [M+Na]⁺.

### Step 2: Preparation of 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 6-aminohexanoate hydrochloride

The title compound was prepared in analogy to example 24, step 2 by using 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 6-((tert-butoxycarbonyl)amino)hexanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.33 g, yield: 97.4%). MS (m/z): [M+H]⁺ calcd for C₂₇H₄₁NO₆, 476.29; found, 476.2. ¹H NMR (400 MHz, D₂O) δ 5.72 (s, 1H), 5.03 (dd, J=36.5, 17.9 Hz, 2H), 4.44 (t, J=6.5 Hz, 1H), 3.04-2.89 (m, 2H), 2.65-2.45 (m, 6H), 2.40-2.23 (m, 2H), 2.19-2.10 (m, 1H), 2.09-1.73 (m, 3H), 1.72-1.61 (m, 6H), 1.59-1.48 (m, 1H), 1.47-1.30 (m, 7H), 1.16-0.98 (m, 2H), 0.83 (d, J=25.7 Hz, 3H).

### Step 3: Preparation of Conjugate of HA and 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 6-aminohexanoate

The title compound was prepared in analogy to example 34, step 3 by using 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 6-aminohexanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.228 g, yield: 76%, DSR=10%). ¹H NMR (400 MHz, D₂O) δ 5.82 (s, 0.1H), 4.97-4.87 (m, 0.2H), 4.72-4.29 (m, 2.1H), 4.18-3.12 (m, 10.2H), 2.64-2.34 (m, 0.6H), 2.27-1.69 (m, 3.6H), 1.66-1.28 (m, 1.4H), 1.24-1.03 (m, 0.2H), 0.89 (s, 0.3H).

### Example 94

### Preparation of Conjugate of HA and 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 6-aminohexanoate

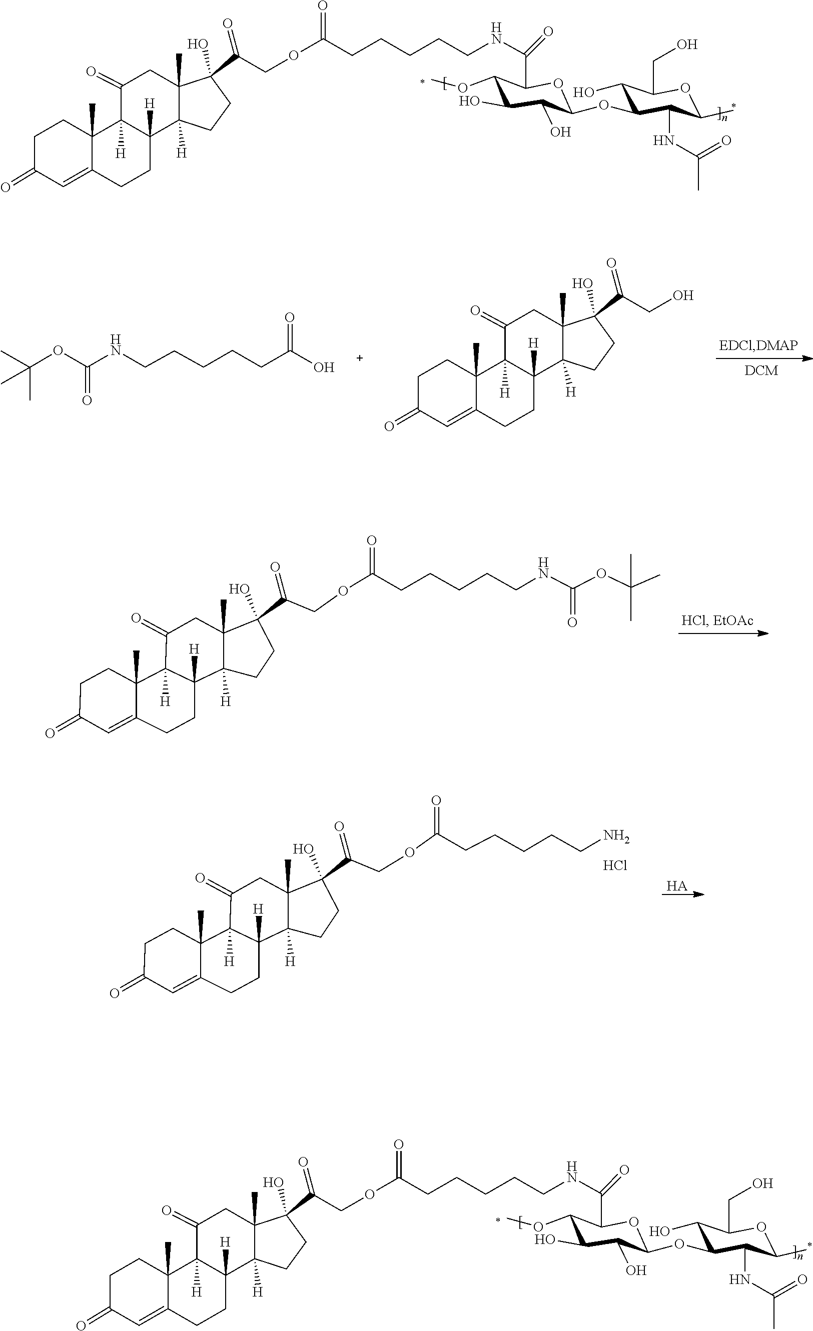

### Step 1: Preparation of 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 6-((tert-butoxycarbonyl)amino)hexanoate

The title compound was prepared in analogy to example 23, step 1 by using (8S,9S,10R,13S,14S,17R)-17-hydroxy-17-(2-hydroxyacetyl)-10,13-dimethyl-1,6,7,8,9,10,12,13,14,15,16,17-dodecahydro-3H-cyclopenta[a]phenanthrene-3,11(2H)-dione instead of (6α,9α,110,160)-6,9-difluoro-11,21-dihydroxy-16,17-[(1-methylethylidene)bis(oxy)]pregna-1,4-diene-3,20-dione was obtained as a white solid (0.403 g, yield: 81.2%). MS (m/z): [M+H]⁺ calcd for C₃₂H₄₇NO₈, 574.33; found, 596.2 [M+Na]⁺.

### Step 2: Preparation of 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 6-aminohexanoate hydrochloride

The title compound was prepared in analogy to example 24, step 2 by using 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 6-((tert-butoxycarbonyl)amino)hexanoate instead of 2-((2S,6αS,6βR,7S,8αaS,8βS,11αR,12αS,12βS)-2,6β-difluoro-7-hydroxy-6α,8α,10,10-tetramethyl-4-oxo-1,2,4,6α,6β,7,8,8α,11α,12,12α,12β-dodecahydro-8βH-naphtho[2′,1′:4,5]indeno[1,2-d][1,3]dioxol-8β-yl)-2-oxoethyl 6-((tert-butoxycarbonyl)amino)hexanoate was obtained as a white solid (0.317 g, yield: 95.2%). MS (m/z): [M+H]⁺ calcd for C₂₇H₃₉NO₆, 474.28; found, 474.2. ¹H NMR (400 MHz, D₂O) δ 5.87 (s, 1H), 5.21-4.90 (m, 2H), 3.10-2.93 (m, 3H), 2.75-2.53 (m, 6H), 2.52-2.28 (m, 5H), 2.21-1.98 (m, 3H), 1.89-1.70 (m, 6H), 1.65-1.26 (m, 7H), 0.67 (s, 3H).

### Step 3: Preparation of Conjugate of HA and 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 6-aminohexanoate

The title compound was prepared in analogy to example 34, step 3 by using 2-((8S,9S,10R,13S,14S,17R)-17-hydroxy-10,13-dimethyl-3,11-dioxo-2,3,6,7,8,9,10,11,12,13,14,15,16,17-tetradecahydro-1H-cyclopenta[a]phenanthren-17-yl)-2-oxoethyl 6-aminohexanoate instead of 2-((8S,9S,10R,13S,14S,17R)-17-hydroxy-10,13-dimethyl-3,11-dioxo-6,7,8,9,10,11,12,13,14,15,16,17-dodecahydro-3H-cyclopenta[a]phenanthren-17-yl)-2-oxoethyl 4-aminobutanoate was obtained as a white solid. (sodium hyaluronate MW 500 KDa 0.24 g, yield: 80%, DSR=16%). ¹H NMR (400 MHz, D₂O) δ 5.87 (s, 0.16H), 4.99-4.86 (m, 0.32H), 4.72-4.35 (m, 2H), 4.28-2.94 (m, 10.48H), 2.71-2.31 (m, 1.76H), 2.30-1.71 (m, 4.44H), 1.70-1.12 (m, 1.12H), 0.67 (s, 0.48H).

### Example 95

### Drug Release of the Drug Delivery System

### Method

The drug release and stability experiments were run using the test compounds. About 2.5±1.0 mg/mL (with regard to conjugate) solution of each test compound was prepared with 10 mM PBS buffer (pH=7.4) in Millipore Amicon® Ultra-0.5 ml 30 k Ultrafiltration centrifuge tube. The solution was kept swelling for 1 h at room temperature and then placed in a shaker at 100 rpm, 37° C. for continuous experiment. At the same time point of each day, samples were centrifuged at 10000 rpm for 1 h. The aliquot was transferred to HPLC vial for analysis. An 0.4 mL of 10 mM PBS buffer was added into the centrifuge tube and continue the experiment.

For the HPLC analysis at each time point, the peak areas of all relevant peaks from the chromatograms were retrieved and the concentration of free drug was calculated. Average release of free drug was calculated based on the sum of free drug and numbers of experiment days. The computing equation is as below.

$$Average release of free drug ( µg / mL / d ) = \frac{the sum of free drug ( µg / mL )}{numbers of experiment days ( d )}$$

The sample degradation rate was calculated based on the concentration and degree of substitution (NMR) for conjugated drug with regard to the initial starting point of the experiment (at t=0). The computing equation is as below.

$$Release rate of conjugate ( \% / d ) = \frac{Average release of free drug ( µg / mL / d )}{Concentration ( mg / mL ) * Degree of substitution ( NMR ) * 1000}$$

### Results

Results of the drug release rate for exemplary drug delivery systems in the present disclosure are shown in Table 1.

TABLE 1

| | Release rate |
|---|---|
| Example No. | (%/day) |
| Example 1 | 14.04 |
| Example 2 | 0.76 |
| Example 3 | 0.25 |
| Example 4 | 0.01 |
| Example 5 | 4.03 |
| Example 6 | 2.34 |
| Example 7 | 14.25 |
| Example 8 | 2.00 |
| Example 9 | 0.22 |
| Example 10 | 0.83 |
| Example 11 | 3.53 |
| Example 12 | 3.30 |
| Example 13 | 0.14 |
| Example 14 | 0.01 |
| Example 15 | 0.97 |
| Example 16 | 1.46 |
| Example 17 | 2.19 |
| Example 18 | 0.90 |
| Example 19 | 17.57 |
| Example 20 | 1.66 |
| Example 21 | 2.05 |
| Example 22 | 0.40 |
| Example 23 | 1.01 |
| Example 24 | 1.64 |
| Example 25 | 1.69 |
| Example 26 | 0.63 |
| Example 27 | 0.34 |
| Example 28 | 16.89 |
| Example 29 | 23.90 |
| Example 30 | 0.94 |
| Example 31 | 1.10 |
| Example 32 | 3.68 |
| Example 33 | 22.57 |
| Example 34 | 3.01 |
| Example 35 | 1.44 |
| Example 36 | 2.89 |
| Example 37 | 1.17 |
| Example 38 | 1.71 |
| Example 39 | 0.71 |
| Example 40 | 2.49 |
| Example 41 | 1.79 |
| Example 42 | 2.88 |
| Example 43 | 1.07 |
| Example 44 | 1.12 |
| Example 45 | 0.40 |
| Example 46 | 2.95 |
| Example 47 | 1.24 |
| Example 48 | 2.00 |
| Example 49 | 0.42 |
| Example 50 | 9.26 |
| Example 51 | 7.21 |
| Example 52 | 4.09 |
| Example 53 | 1.49 |
| Example 54 | 1.26 |
| Example 55 | 1.20 |
| Example 56 | 0.77 |
| Example 57 | 0.23 |
| Example 58 | 0.25 |
| Example 59 | 0.02 |
| Example 60 | 15.04 |
| Example 61 | 7.26 |
| Example 62 | 0.14 |
| Example 63 | 0.05 |
| Example 64 | 7.61 |
| Example 65 | 7.52 |
| Example 66 | 11.18 |
| Example 67 | 5.91 |
| Example 68 | 8.02 |
| Example 69 | 4.62 |
| Example 70 | 3.49 |
| Example 71 | 1.55 |
| Example 72 | 7.86 |
| Example 73 | 13.44 |
| Example 74 | 4.06 |
| Example 75 | 4.80 |
| Example 76 | 2.66 |
| Example 77 | 4.81 |
| Example 78 | 8.92 |
| Example 79 | 4.42 |
| Example 80 | 1.99 |
| Example 81 | 1.71 |
| Example 82 | 2.30 |
| Example 83 | 2.15 |
| Example 84 | 3.73 |
| Example 85 | 7.65 |
| Example 86 | 6.93 |
| Example 87 | 6.96 |
| Example 88 | 3.19 |
| Example 89 | 8.12 |
| Example 90 | 4.56 |
| Example 91 | 11.66 |
| Example 92 | 8.21 |
| Example 93 | 7.67 |
| Example 94 | 2.85 |

### Example 96

### In Vivo Systemic Exposure and Joint Retention Assay

LEWIS rats were employed in this study. Based on body weights, animals were randomized and assigned into G1 group (9 rats) and G2 group (9 rats). Rats in G1 group and G2 group were respectively treated with intra-articular injections of Example 2 (HA MW 1000 KDa) and Example 2 (HA MW 2000 KDa) by giving single injections of 100 μl of a solution with 1.5 mg of Example 2 (HA MW 1000 KDa) or Example 2 (HA MW 2000 KDa) per joint.

At 3, 7, 14, 30 and 60 days of injections, blood samples were collected from these rats of each group. Approximately 1.0 mL of blood samples per point were collected with EDTA-K2 anticoagulant tubes to analyze the level of triamcinolone acetonide in plasma. Apart from the systemic exposure evaluation, knee samples were also collected to estimate the level of triamcinolone acetonide in joint tissues. At the last time point (14d, 30d and 60d), the blood samples were collected then the animals were sacrificed, both knee joints were dissected and the synovial membrane was removed. After rinsed with saline and drained with filter paper, the synovial membrane was weighed. All samples were stored at −70˜−80° C. until analyzed by LC-MS/MS.

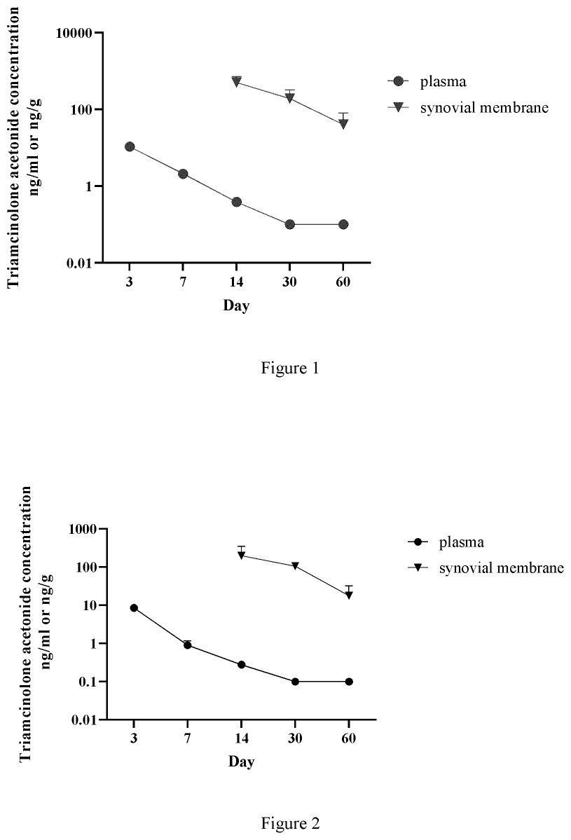

The individual plasma and synovium time-concentration data of triamcinolone acetonide (for plasma, the concentration of triamcinolone acetonide is expressed in ng/ml; for synovium, the concentration of triamcinolone acetonide is expressed in ng/g) after a single IA dose of Example 2 (HA MW 1000 KDa) and Example 2 (HA MW 2000 KDa) were shown in FIG. 1 and FIG. 2, respectively. The results indicate that the concentrations of triamcinolone acetonide in plasma was much lower than that in synovium and triamcinolone acetonide could still be detected after 2 months post IA injection.

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

What claimed is:

1. A drug delivery system for locally delivering a therapeutic agent at a controlled rate, the drug delivery system comprising:
a biopolymer comprising at least a first binding group BG1 selected from the group consisting of carboxylic group, amino group and a combination thereof;
a therapeutic agent comprising at least a second binding group BG2 selected from the group consisting of hydroxyl group, carboxylic group, amino group, amide group, amine group and a combination thereof; and
a linker covalently linking the biopolymer to the therapeutic agent and capable of retaining the therapeutic agent in the location of administration;
wherein the linker comprises a structure of formula (I): 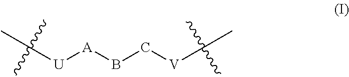
wherein
U is connected to the biopolymer through BG1 such that at least one amide linkage is formed, and U is selected from —N(R¹)— or 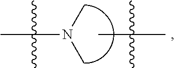
wherein 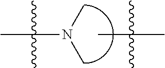
is a nitrogen-containing heteroaryl or a nitrogen-containing heterocyclyl optionally comprising one or more additional heteroatoms selected from N, O or S;
A is selected from a direct bond, alkyl and —(CH₂CH₂O)ₘ—, wherein said alkyl is optionally substituted with one or more R² groups;
B is selected from the group consisting of a direct bond, cycloalkyl, heterocyclyl, aryl, and heteroaryl, wherein each of cycloalkyl, heterocyclyl, aryl and heteroaryl is optionally substituted with one or more R³ groups;
C is selected from a direct bond, alkyl, or —[C(═O)NHCH₂]ₙ—, wherein said alkyl is optionally substituted with one or more R⁴ groups;
V is connected to the therapeutic agent through BG2 such that at least one linkage selected from the group consisting of a direct bond, ester, carbonate, carbamate, —C(═O)NHCH₂O— or —C(═O)OCH₂O— is formed, and V is selected from the group consisting of —C(═O)—, —OC(═O)—, —NHC(═O)—, —C(═O)NHCH₂— and —C(═O)OCH₂—;
R¹ is selected from the group consisting of hydrogen, alkyl, alkenyl and alkynyl;
each of R², R³, and R⁴ is independently selected from the group consisting of halogen, hydroxyl, amino, cyano, alkyl, alkoxyl, and —C(═O)OR⁵;
R⁵ is an alkyl;
m is an integer from 0 to 5; and
n is an integer from 1 to 4,
provided that when the biopolymer is chondroitin sulfate, V is not —C(═O)—.

2. The drug delivery system of claim 1, wherein BG1 is carboxylic group and U is —N(R¹)—, such that an amide linkage is formed.

3. The drug delivery system of claim 1, wherein BG1 is carboxylic group and U is 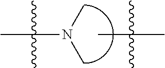
such that an amide linkage is formed.

4. The drug delivery system of claim 3, wherein U is 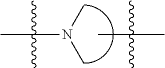
selected from the group consisting of:

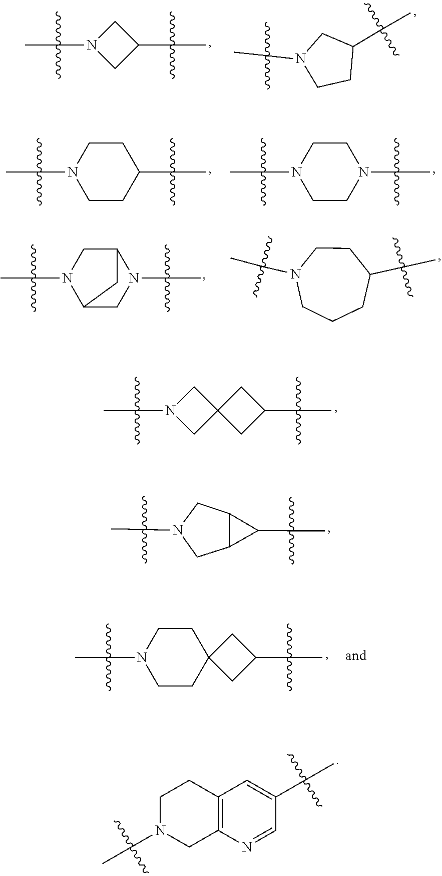

5. The drug delivery system of claim 2, wherein R¹ is hydrogen.

6. The drug delivery system of claim 1, wherein BG2 is a hydroxyl group, V is selected from one of the following:
(a) —C(═O)— which is connected to the therapeutic agent through BG2 such that an ester linkage is formed;
(b) —OC(═O)— which is connected to the therapeutic agent through BG2 such that a carbonate linkage is formed;
(c) —NHC(═O)— which is connected to the therapeutic agent through BG2 such that a carbamate linkage is formed;
(d) —C(═O)NHCH₂— which is connected to the therapeutic agent through BG2 such that —C(═O)NHCH₂O— linkage is formed; or
(e) —C(═O)OCH₂— which is connected to the therapeutic agent through BG2 such that —C(═O)OCH₂O— linkage is formed.

7. The drug delivery system of claim 1, wherein A is a direct bond.

8. The drug delivery system of claim 1, wherein A is alkyl optionally substituted with one or more R² groups.

9. The drug delivery system of claim 8, wherein each R² is independently selected from alkyl or —C(═O)OR⁵.

10. The drug delivery system of claim 1, wherein A is —(CH₂CH₂O)ₘ—.

11. The drug delivery system of claim 1, wherein B is a direct bond.

12. The drug delivery system of claim 1, wherein B is cycloalkyl.

13. The drug delivery system of claim 12, wherein B is selected from the group consisting of cyclobutyl, cyclopentyl, cyclohexyl, and bicycle[2.2.2]octyl.

14. The drug delivery system of claim 1, wherein B is aryl or heteroaryl.

15. The drug delivery system of claim 14, wherein B is selected from the group consisting of phenyl, pyridyl, and furyl.

16. The drug delivery system of claim 1, wherein A is a direct bond, and B is selected from the group consisting of a direct bond, cycloalkyl, and aryl.

17. The drug delivery system of claim 1, wherein A is alkyl optionally substituted with one or more R² groups, and B is selected from the group consisting of a direct bond, aryl, and heteroaryl.

18. The drug delivery system of claim 1, wherein A is —(CH₂CH₂O)ₘ—, B is a direct bond.

19. The drug delivery system of claim 1, wherein C is a direct bond.

20. The drug delivery system of claim 1, wherein C is alkyl optionally substituted with one or more R⁴ groups.

21. The drug delivery system of claim 20, wherein each R⁴ is independently selected from alkyl or —C(═O)OR⁵.

22. The drug delivery system of claim 1, wherein C is —[C(═O)NHCH₂]ₙ—.

23. The drug delivery system of claim 1, wherein A is a direct bond, and B is a direct bond or cycloalkyl, and C is selected from the group consisting of a direct bond or alkyl optionally substituted with one or more R⁴ groups.

24. The drug delivery system of claim 1, wherein A is alkyl optionally substituted with one or more R² groups, B is selected from the group consisting of a direct bond, aryl, and heteroaryl, and C is selected from a direct bond, alkyl optionally substituted with one or more R⁴ groups, or —[C(═O)NHCH₂]ₙ—.

25. The drug delivery system of claim 1, wherein A is —(CH₂CH₂O)ₘ—, B is a direct bond, and C is alkyl optionally substituted with one or more R⁴ groups.

26. The drug delivery system of claim 1, wherein the linker comprises a structure of formula (Ia) to (Id): 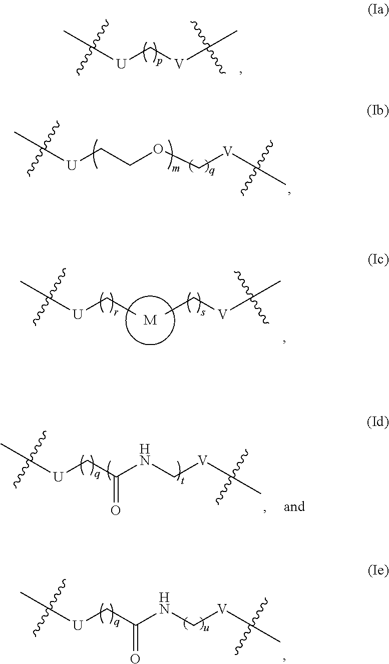
wherein,
U and V are as defined in claim 1;
M is selected from the group consisting of cycloalkyl, heterocyclyl, aryl, and heteroaryl, each of which is optionally substituted with one or more R³ groups;
each of 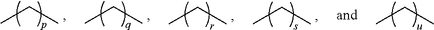
is optionally substituted with one or more group independently selected from alkyl or —C(═O)OCH₃;
p is an integer ranging from 0 to 10;
m and t are independently an integer ranging from 1 to 5;
q, r and s are independently an integer ranging from 0 to 5; and
t is an integer ranging from 1 to 5.

27. The drug delivery system of claim 26, wherein M is selected from the group consisting of cyclobutyl, cyclopentyl, cyclohexyl, bicycle[2.2.2]octyl, phenyl, pyridyl and furyl.

28. The drug delivery system of claim 26, wherein the linker comprises a structure selected from the group consisting of: 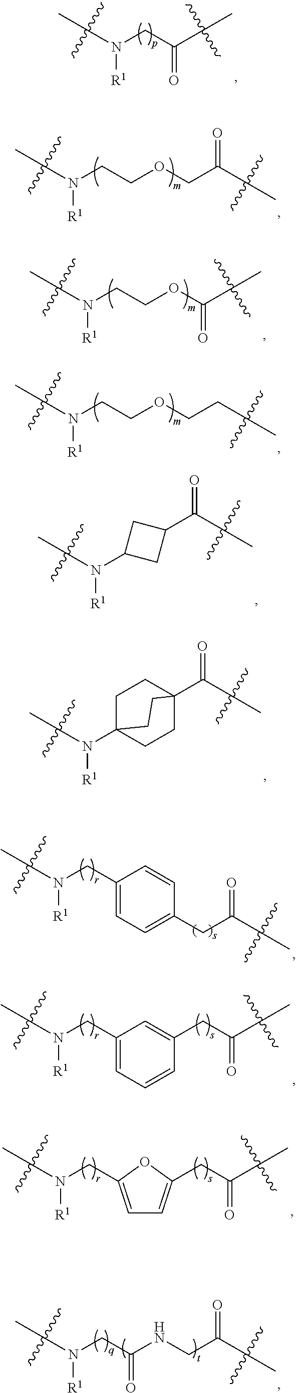 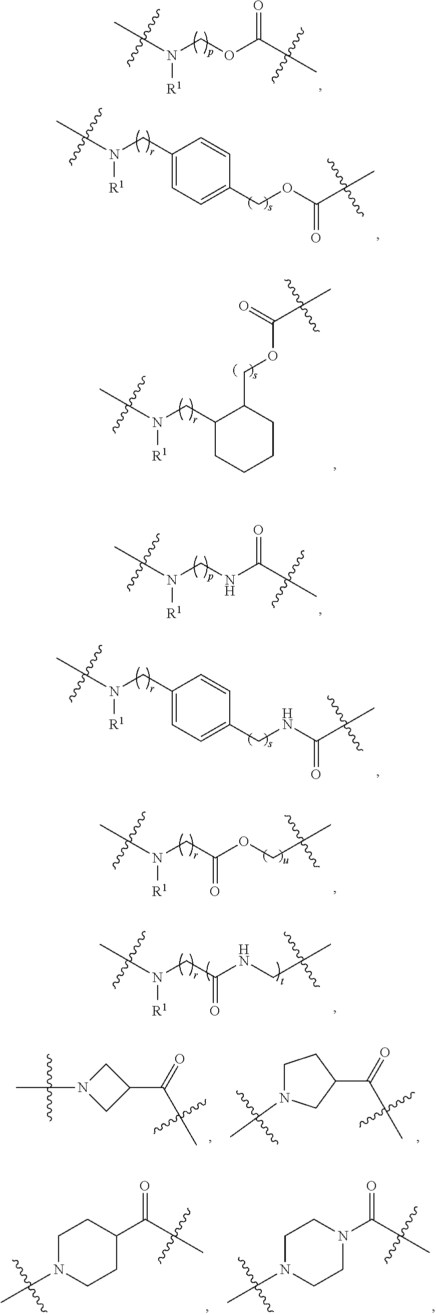 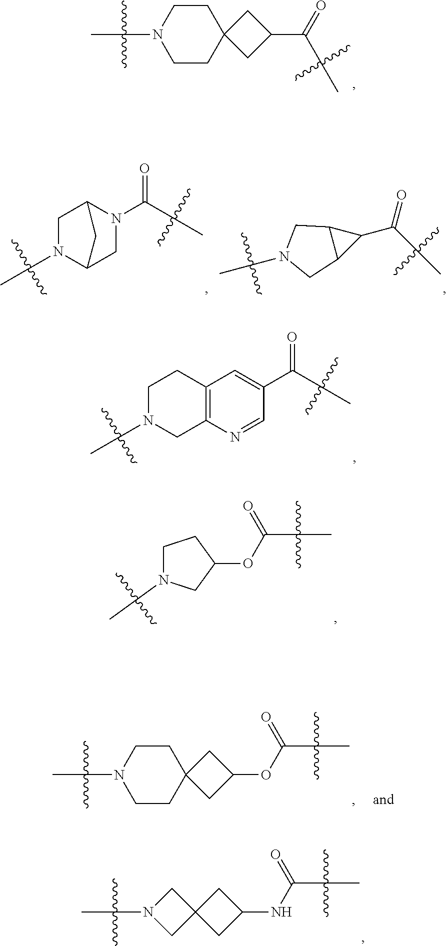
wherein each of 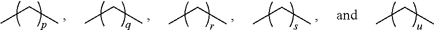
is optionally substituted with one or more group independently selected from alkyl or —C(═O)OCH₃.

29. The drug delivery system of any one of claims 1-28, wherein the biopolymer is selected from the group consisting of hyaluronic acid, chitosan, chondroitin sulfate, or derivatives thereof.

30. The drug delivery system of claim 29, wherein the biopolymer is hyaluronic acid.

31. The drug delivery system of claim 29, wherein the biopolymer is chondroitin sulfate.

32. The drug delivery system of any one of claims 1-31, wherein the therapeutic agent is selected from the group consisting of triamcinolone acetonide, meprednisone, prednisolone, hydrocortisone, cortisone, fluocinonide, methylprednisolone, betamethason, and dexamethasone.

33. The drug delivery system of claim 32, wherein the biopolymer is hyaluronic acid.

34. The drug delivery system of any one of claims 1-33, selected from the group consisting of:

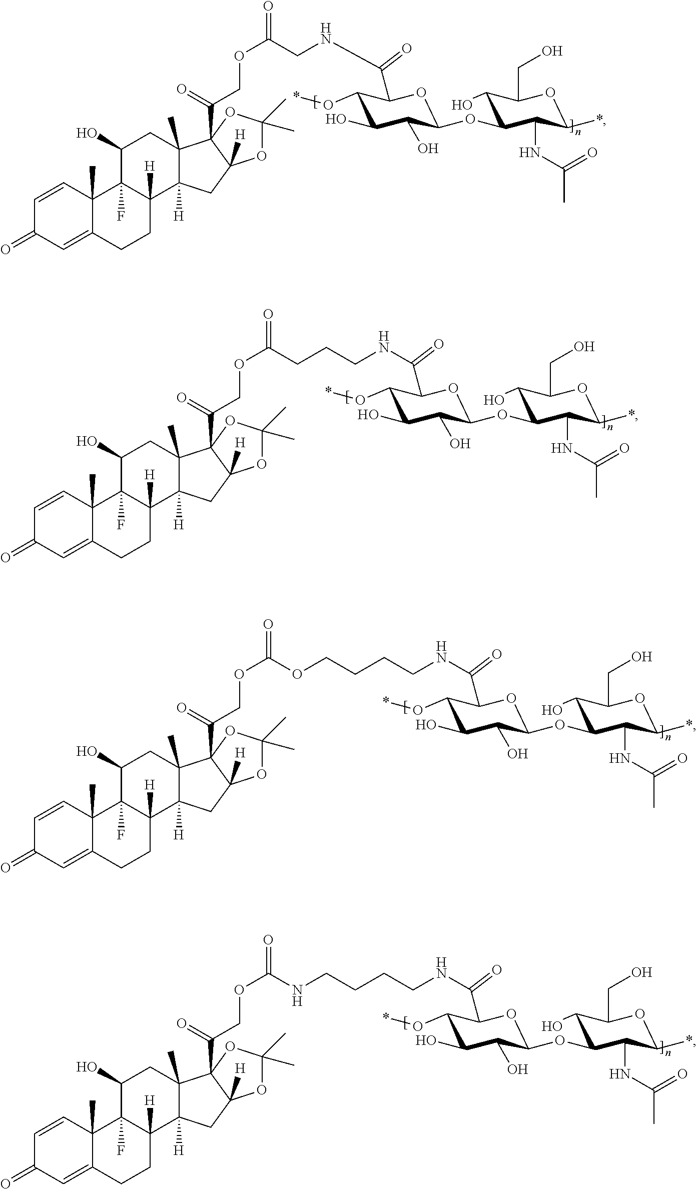

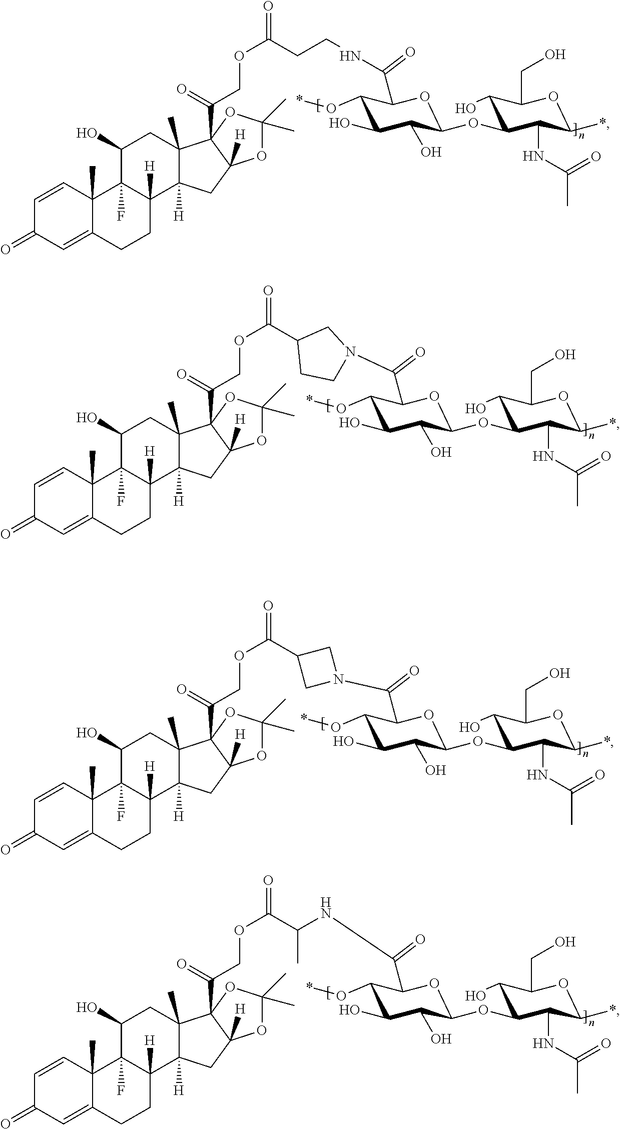

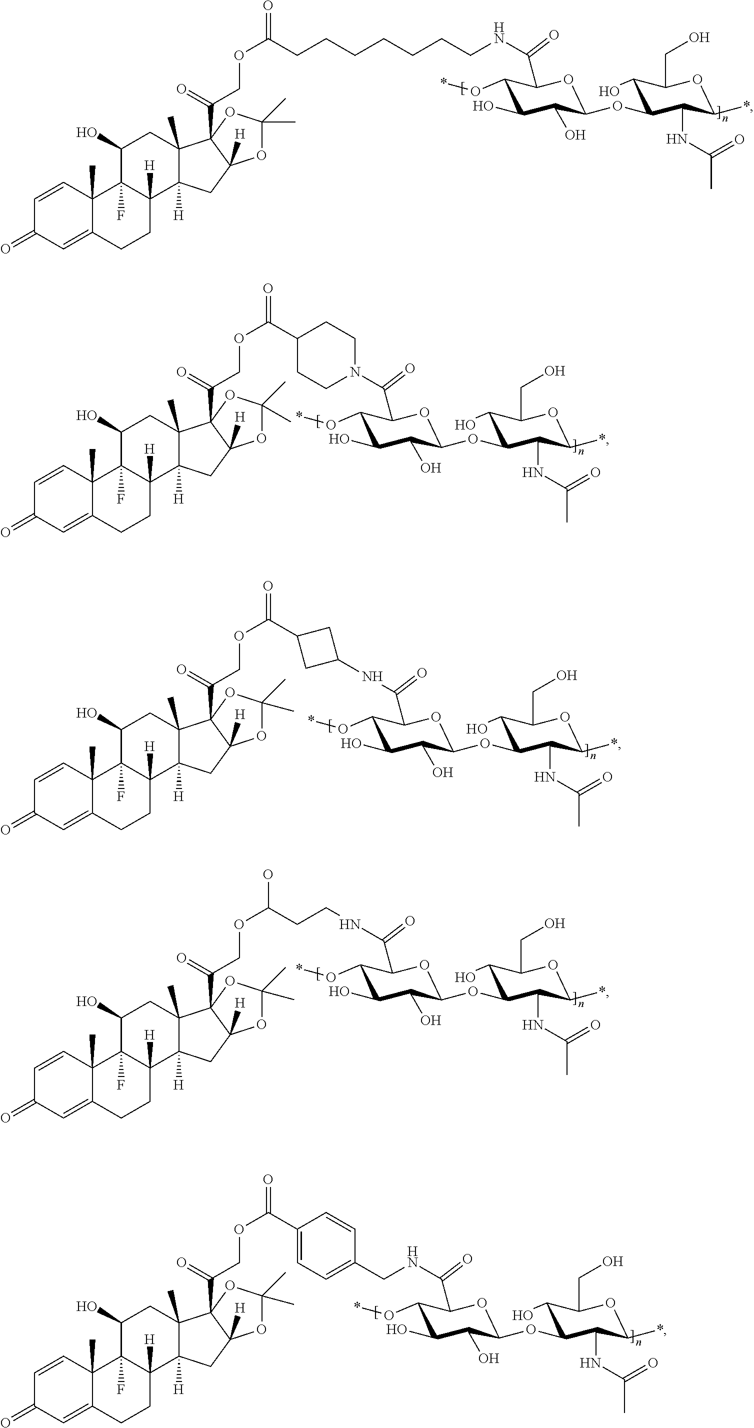

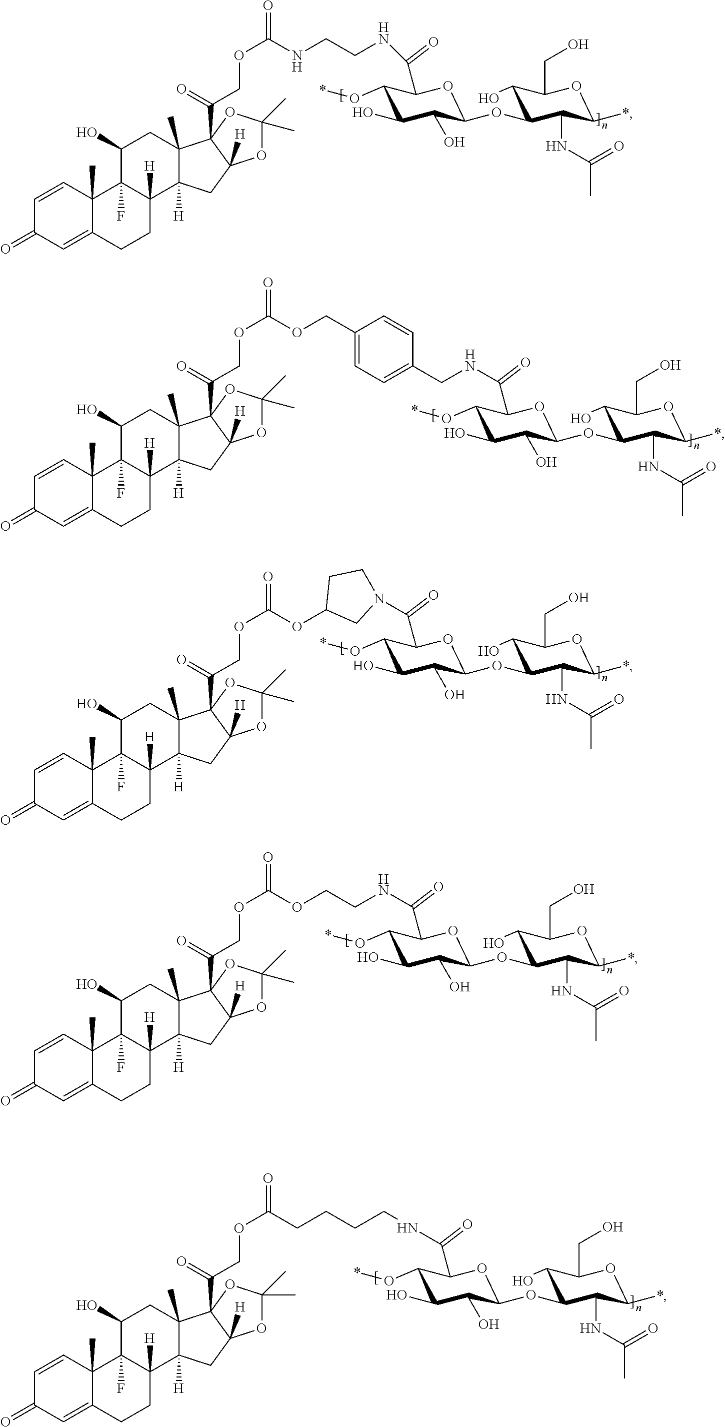

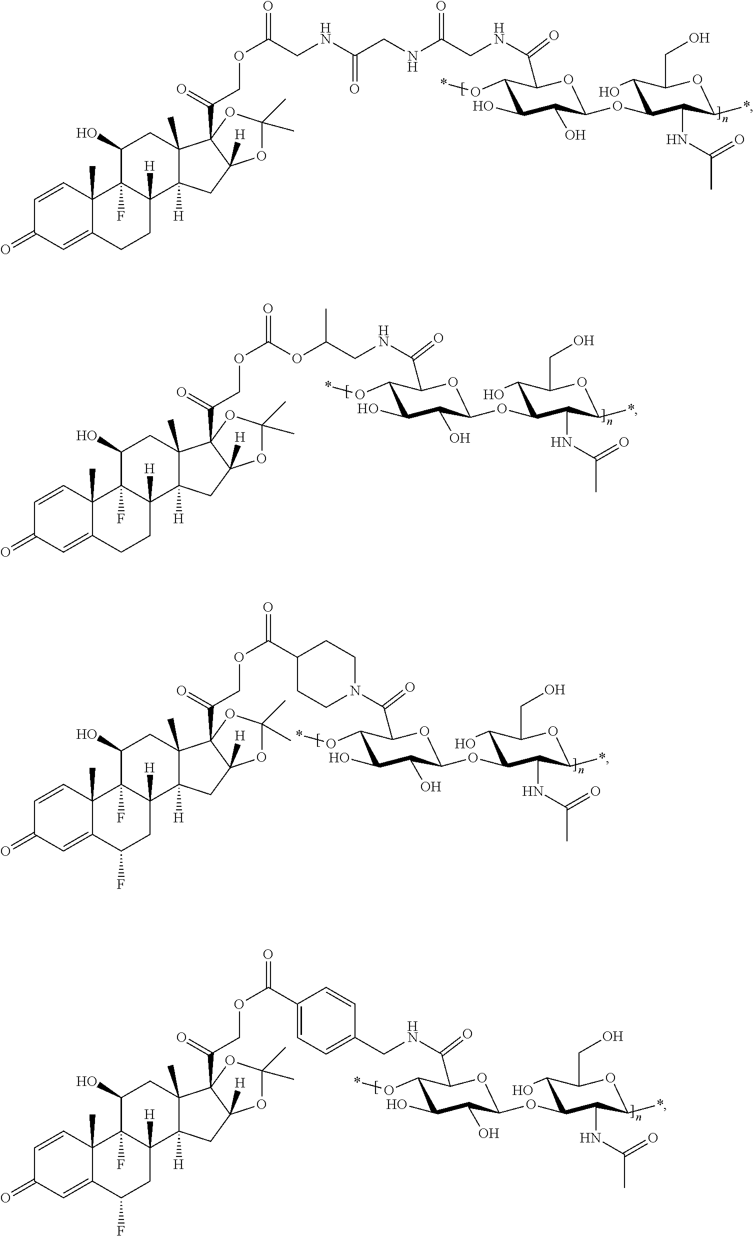

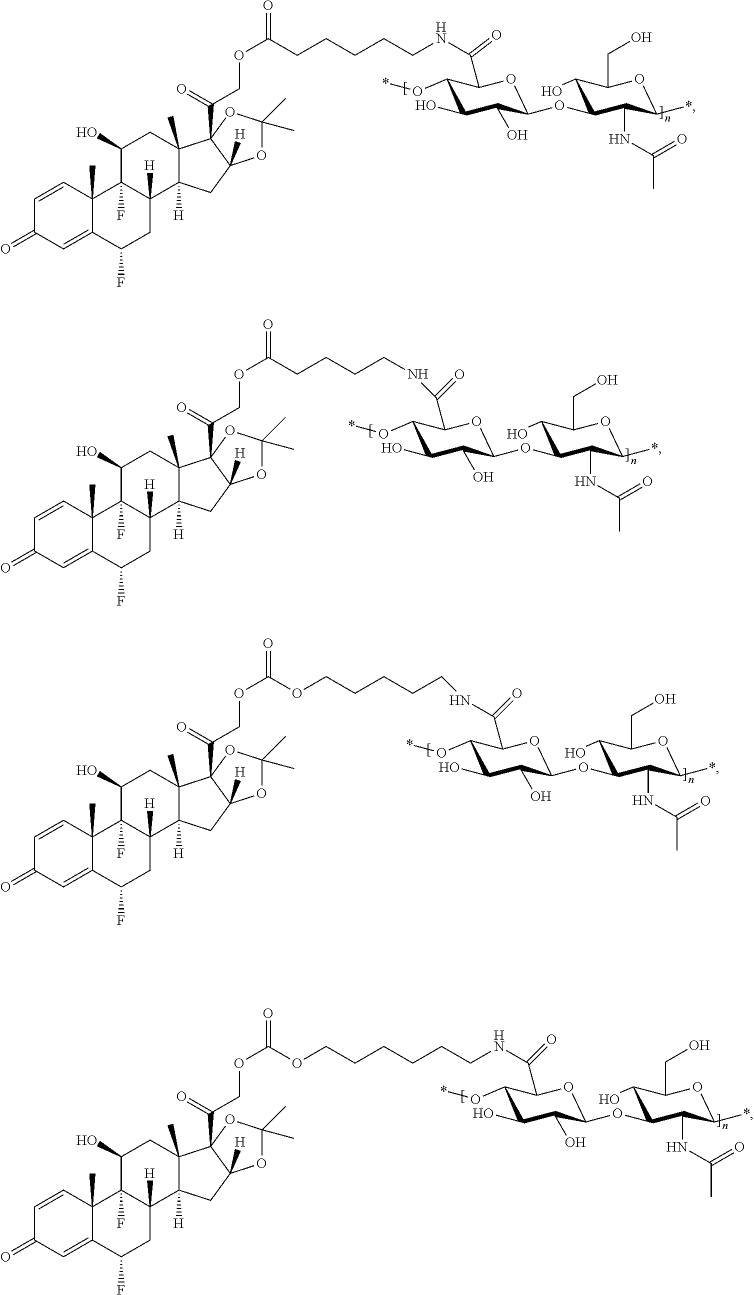

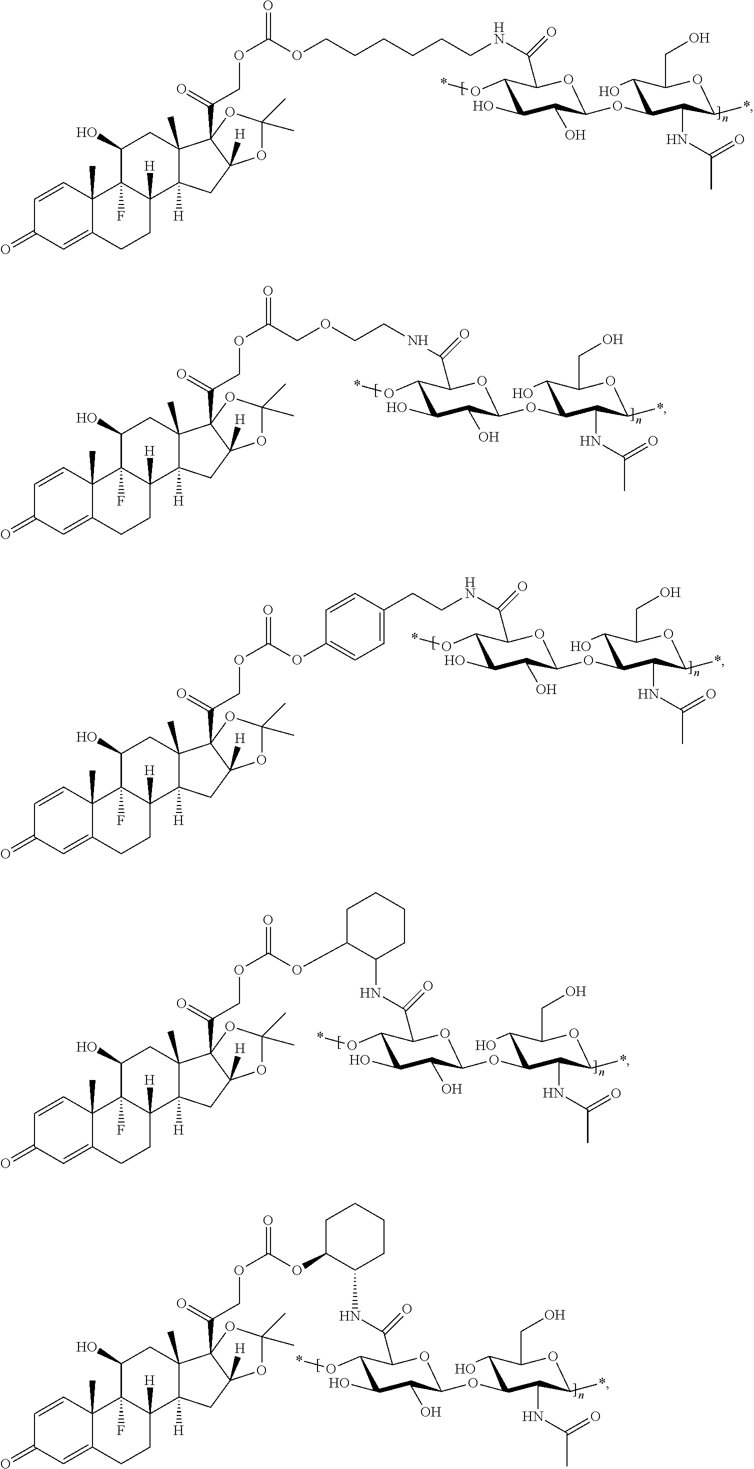

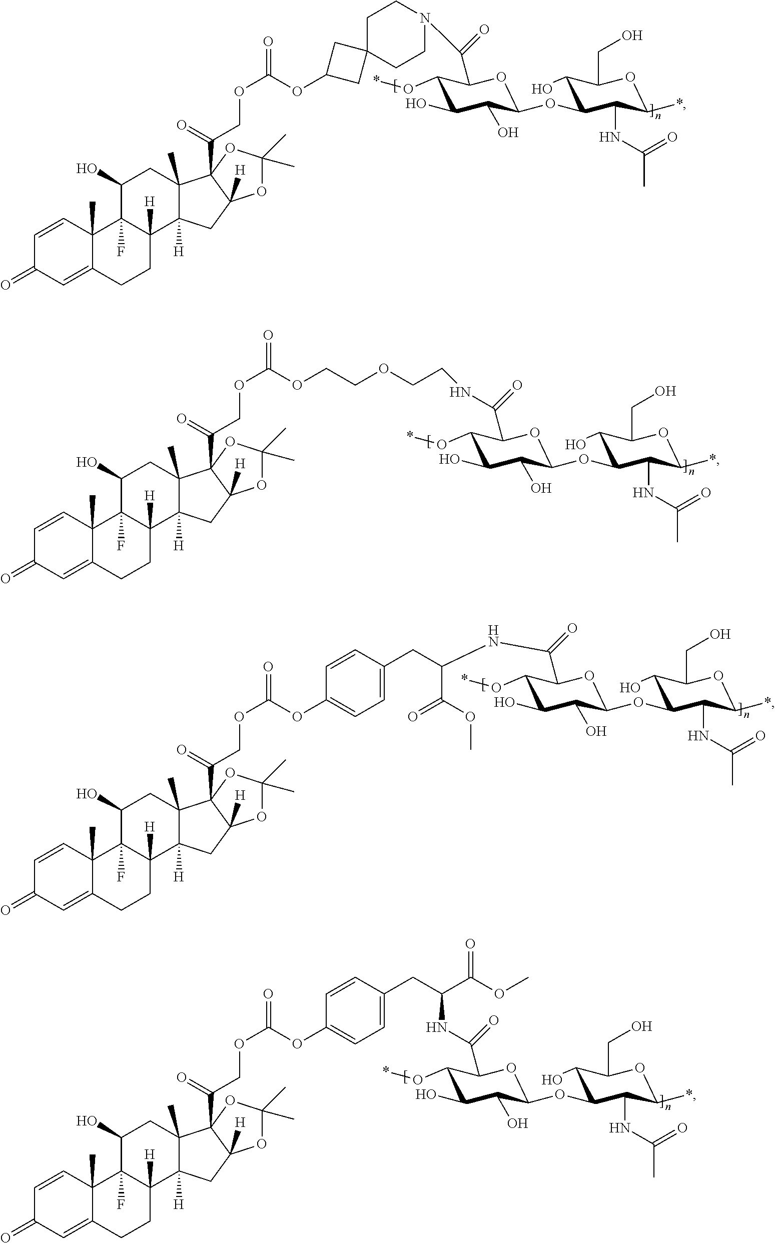

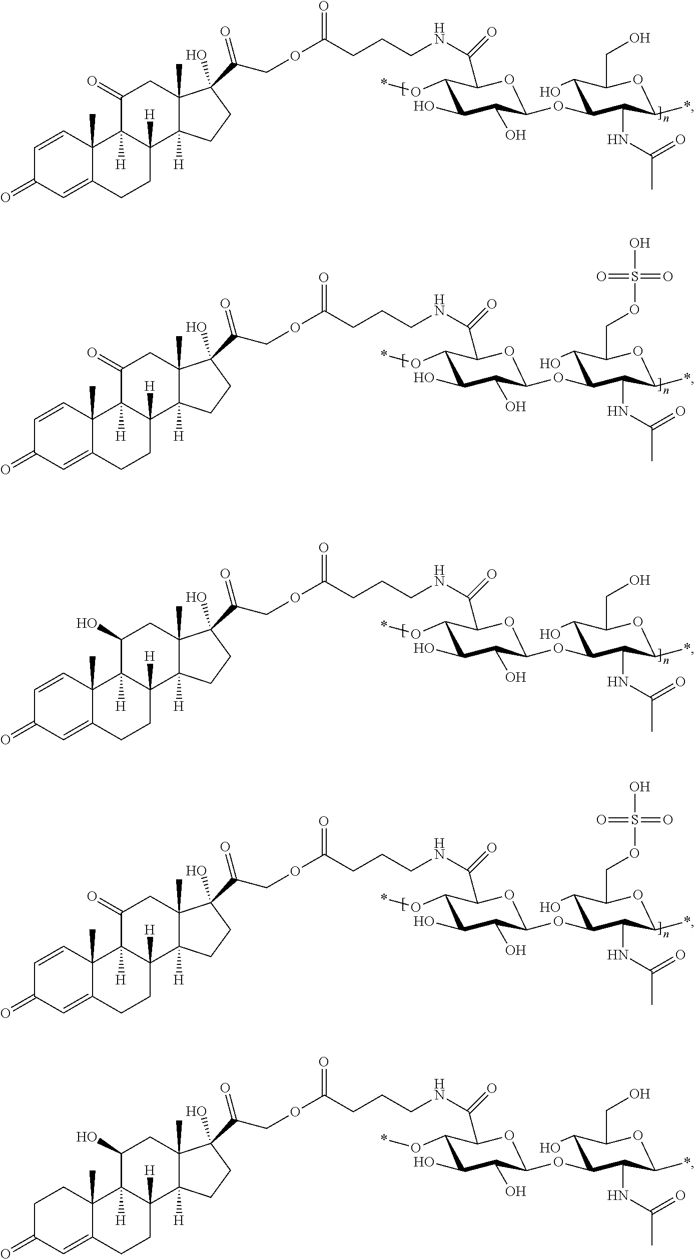

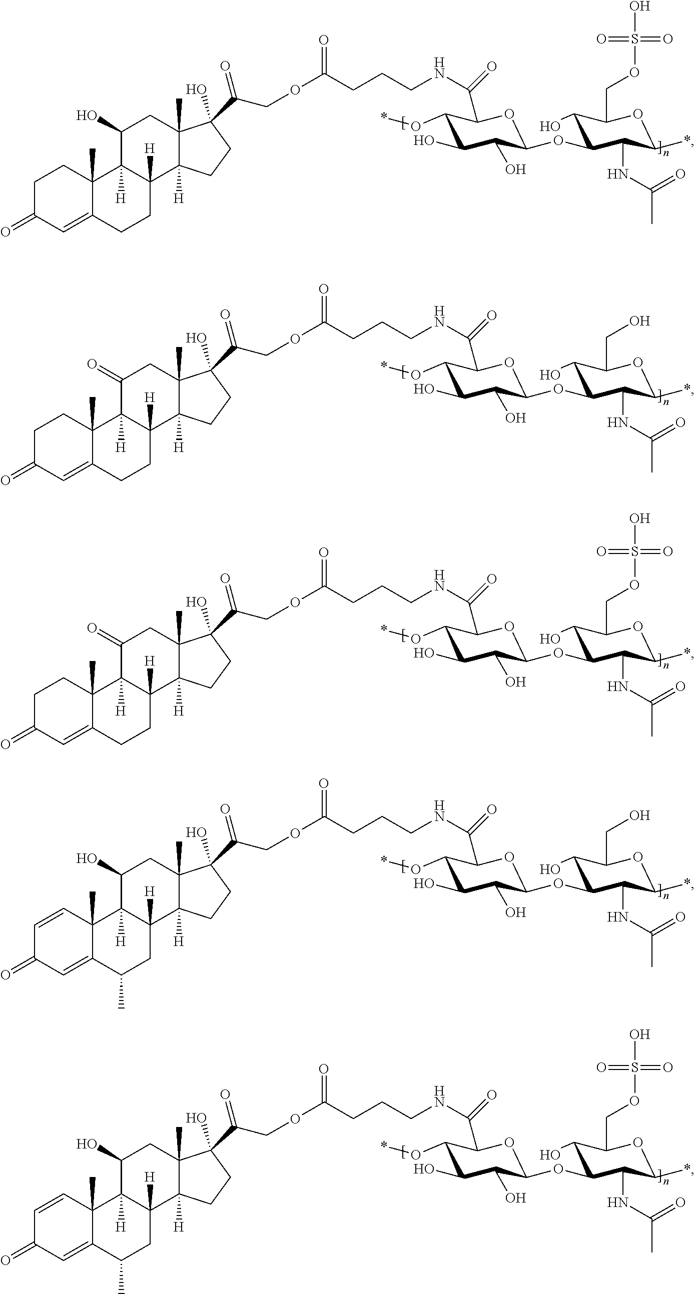

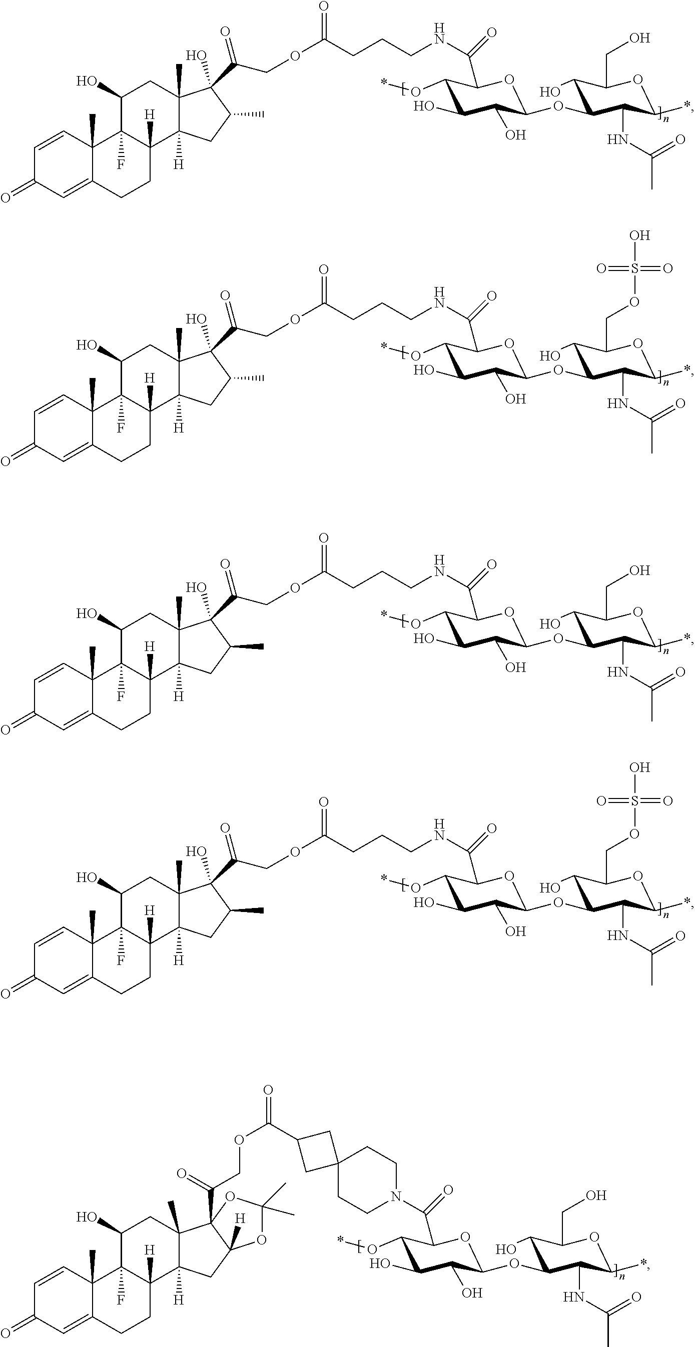

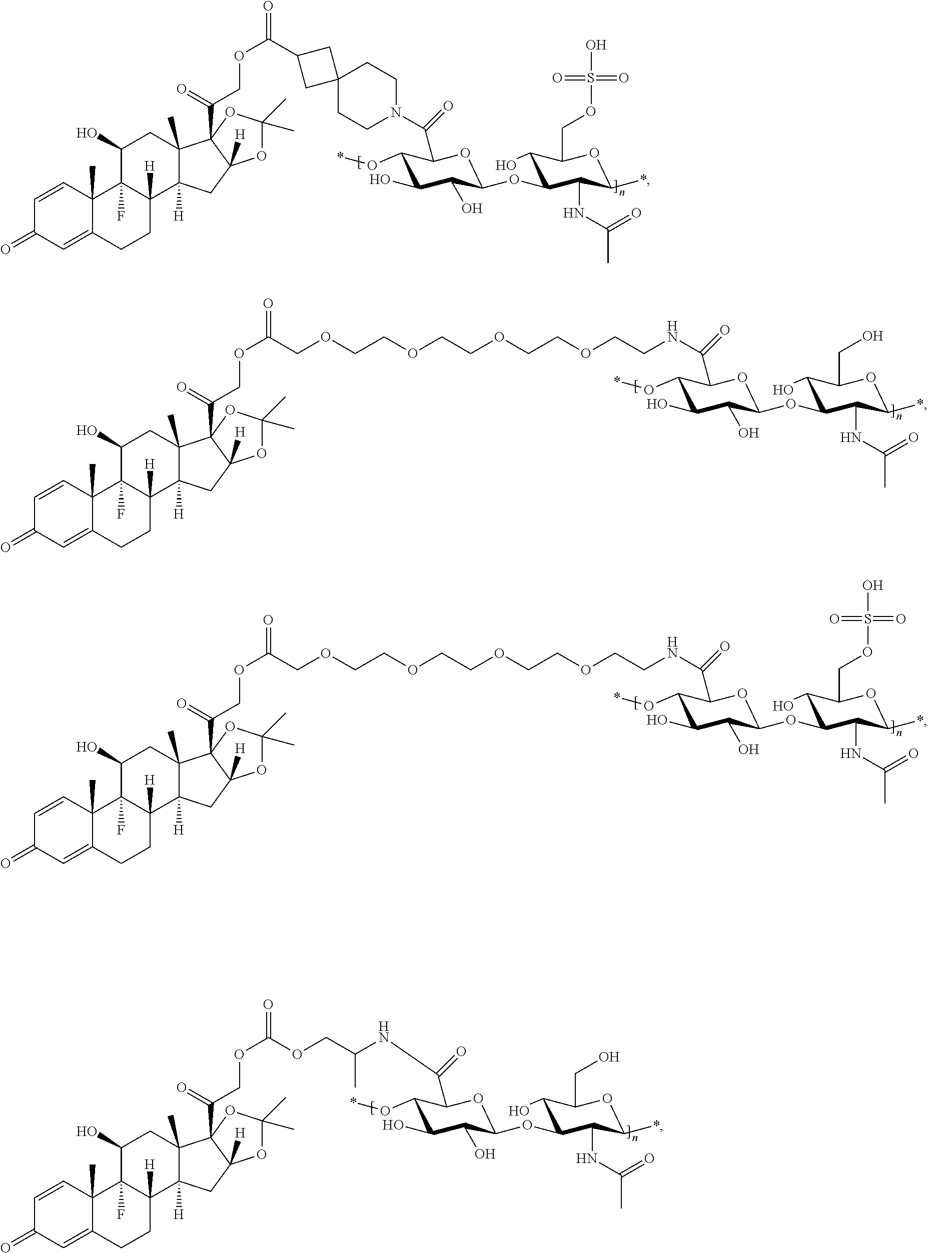

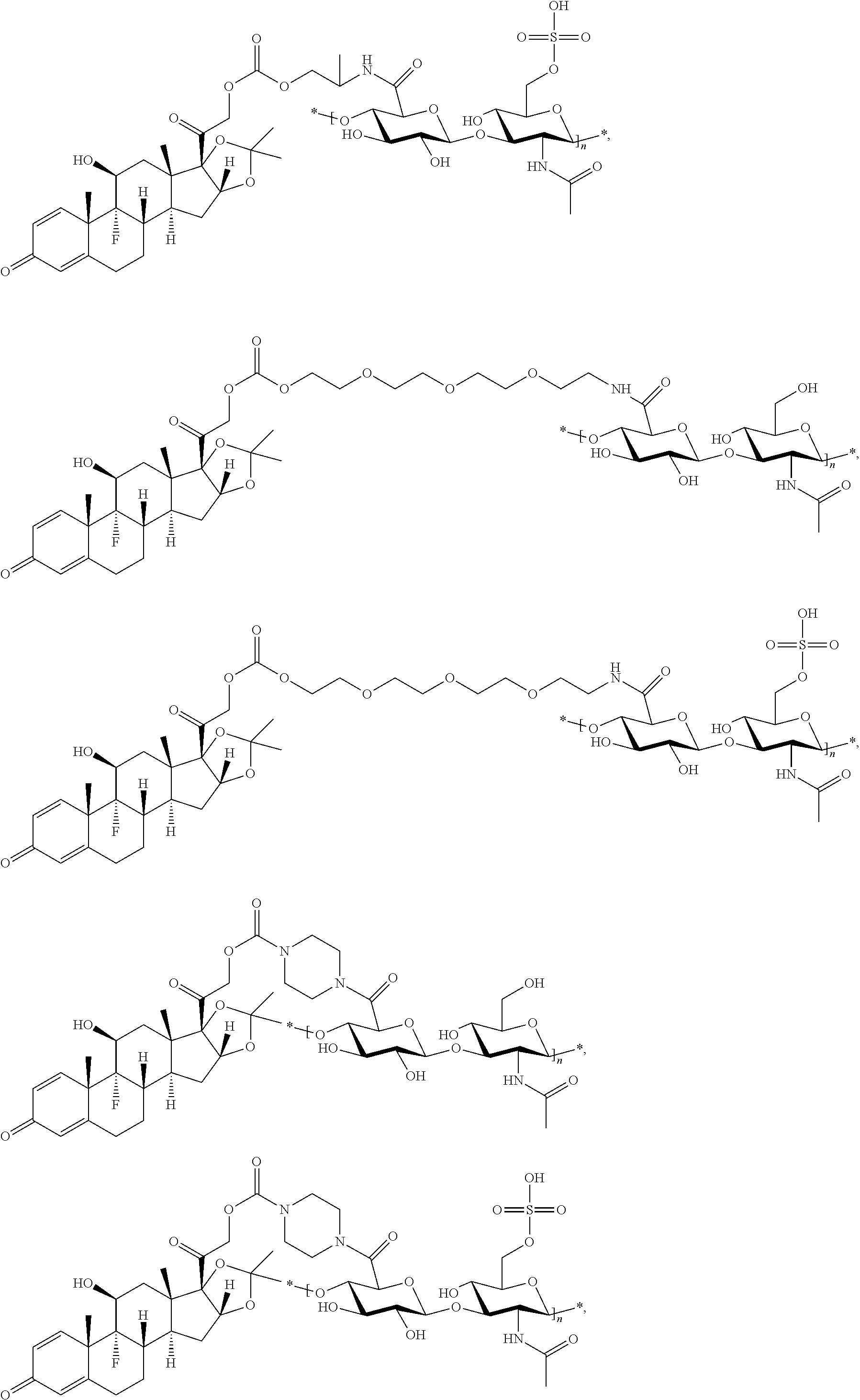

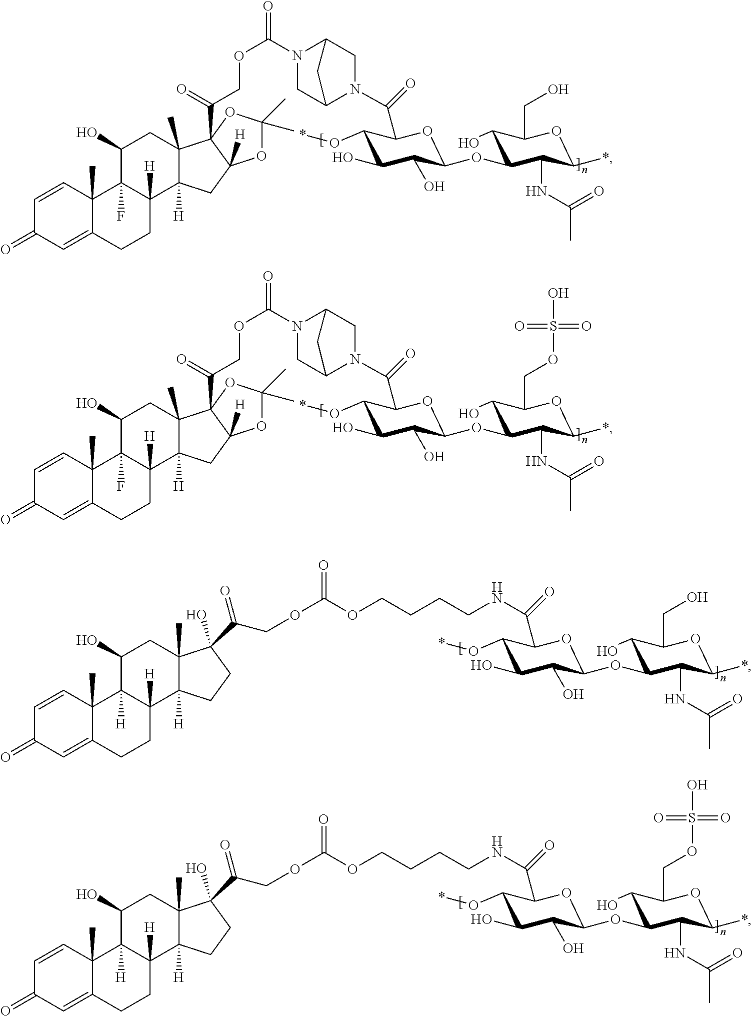

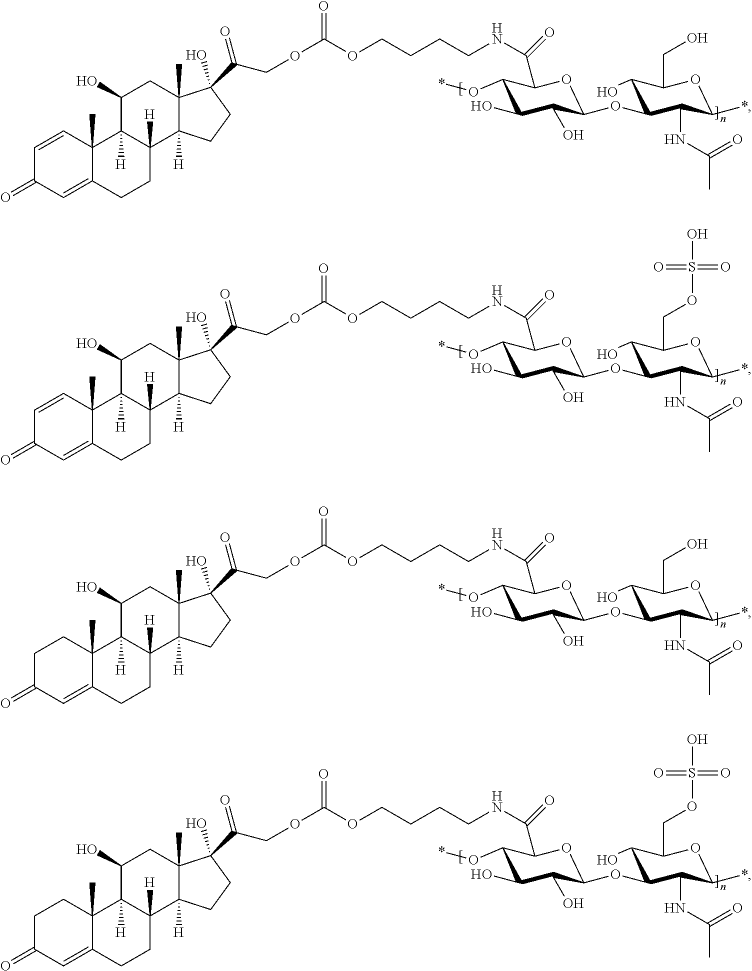

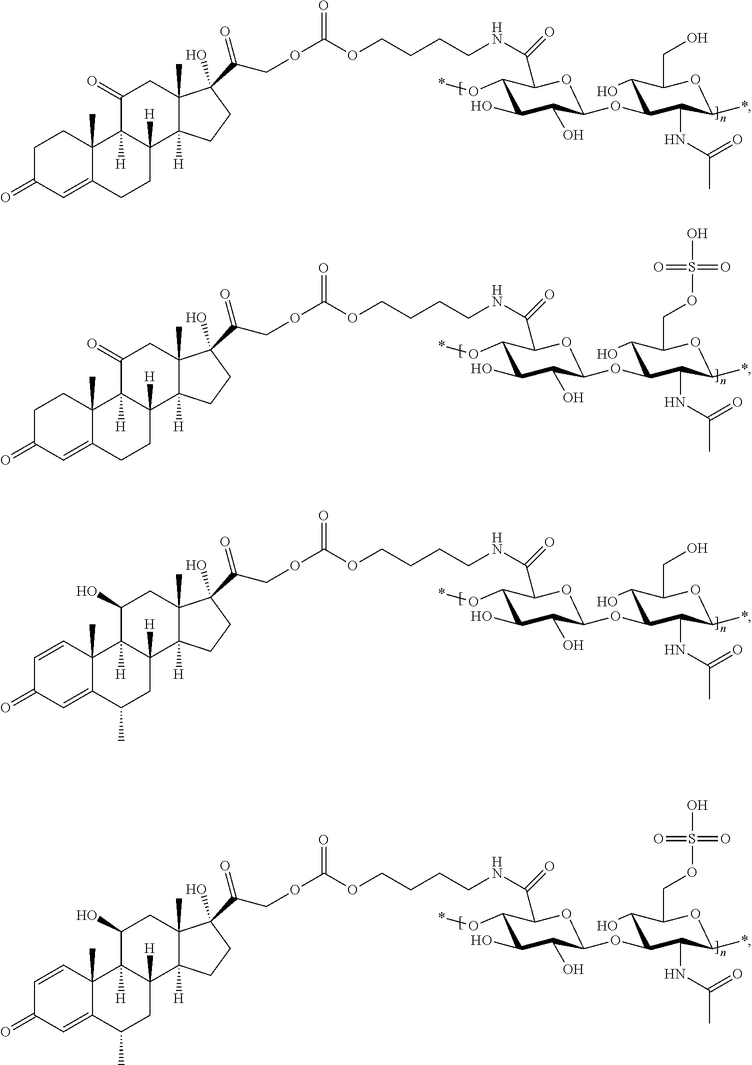

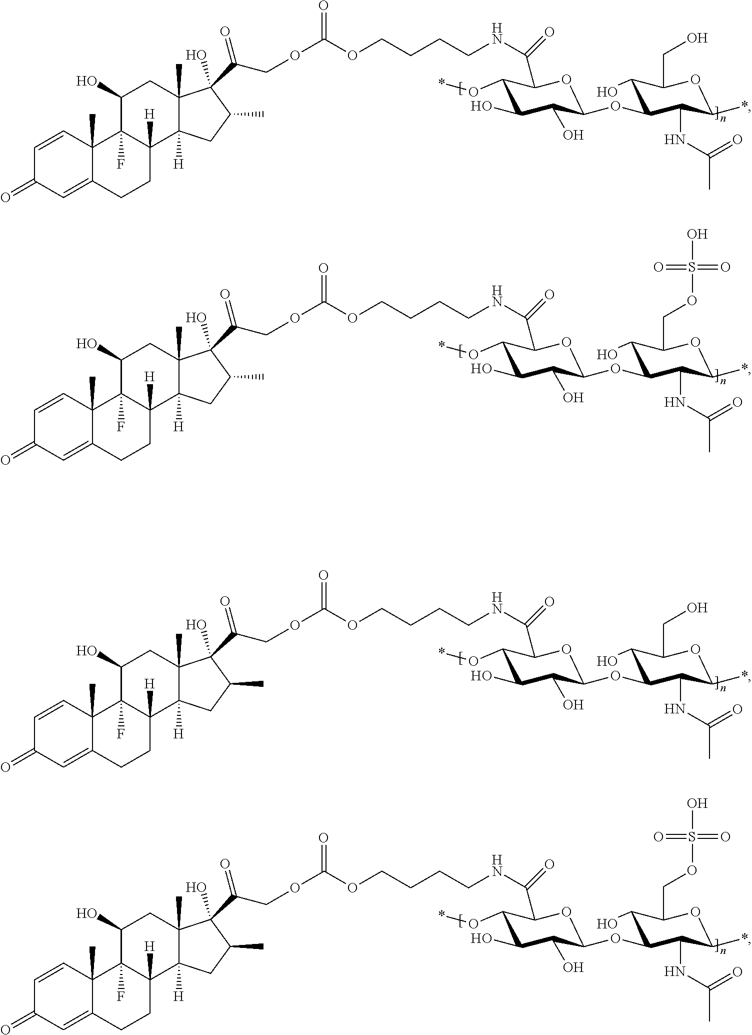

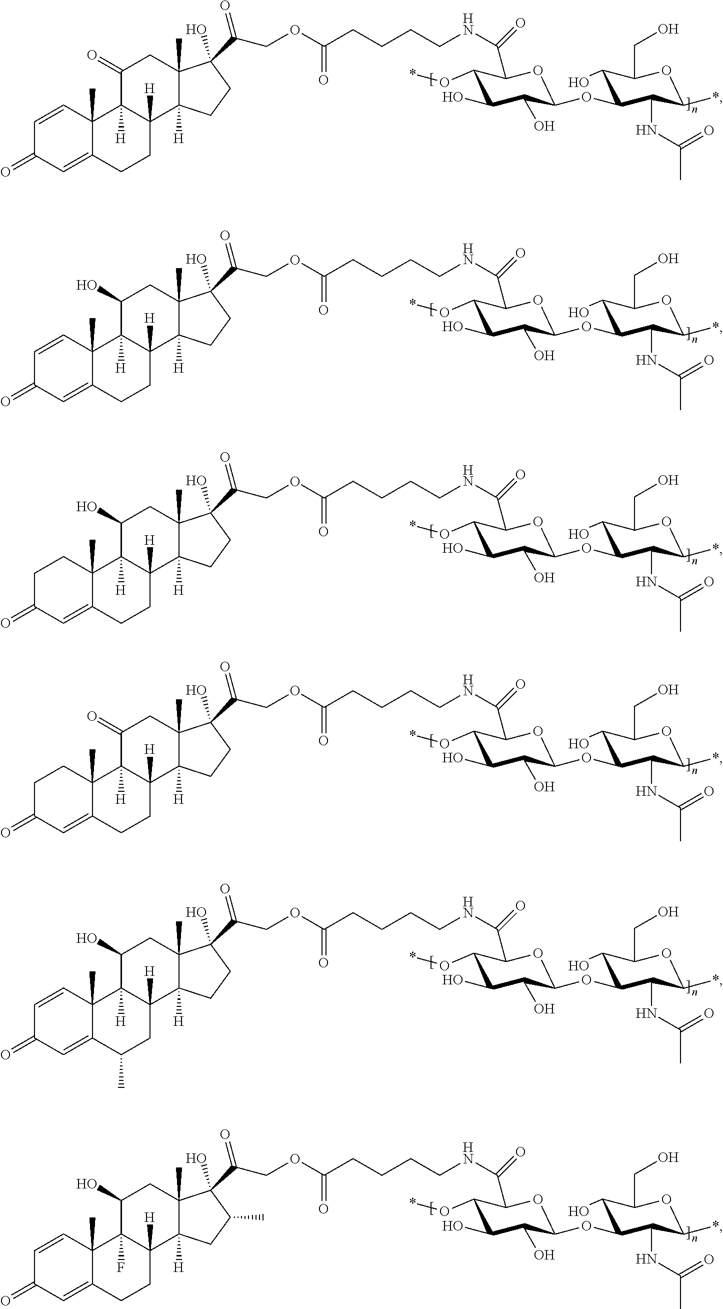

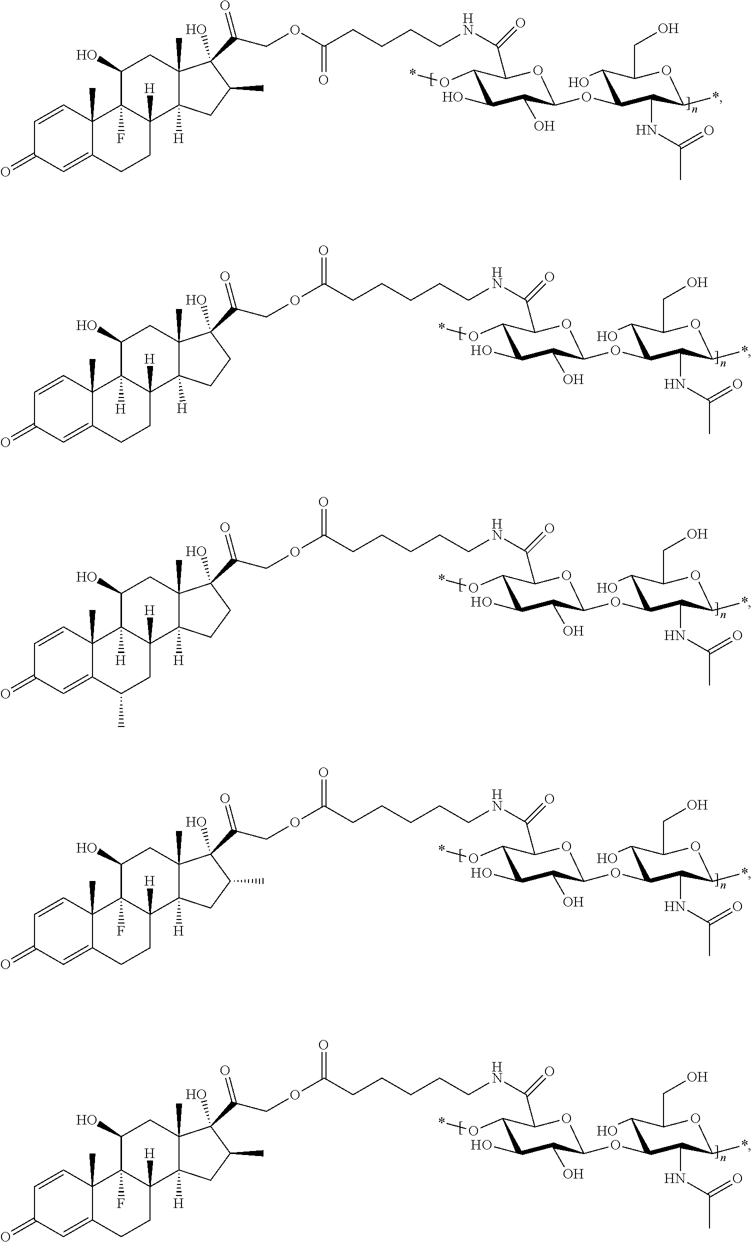

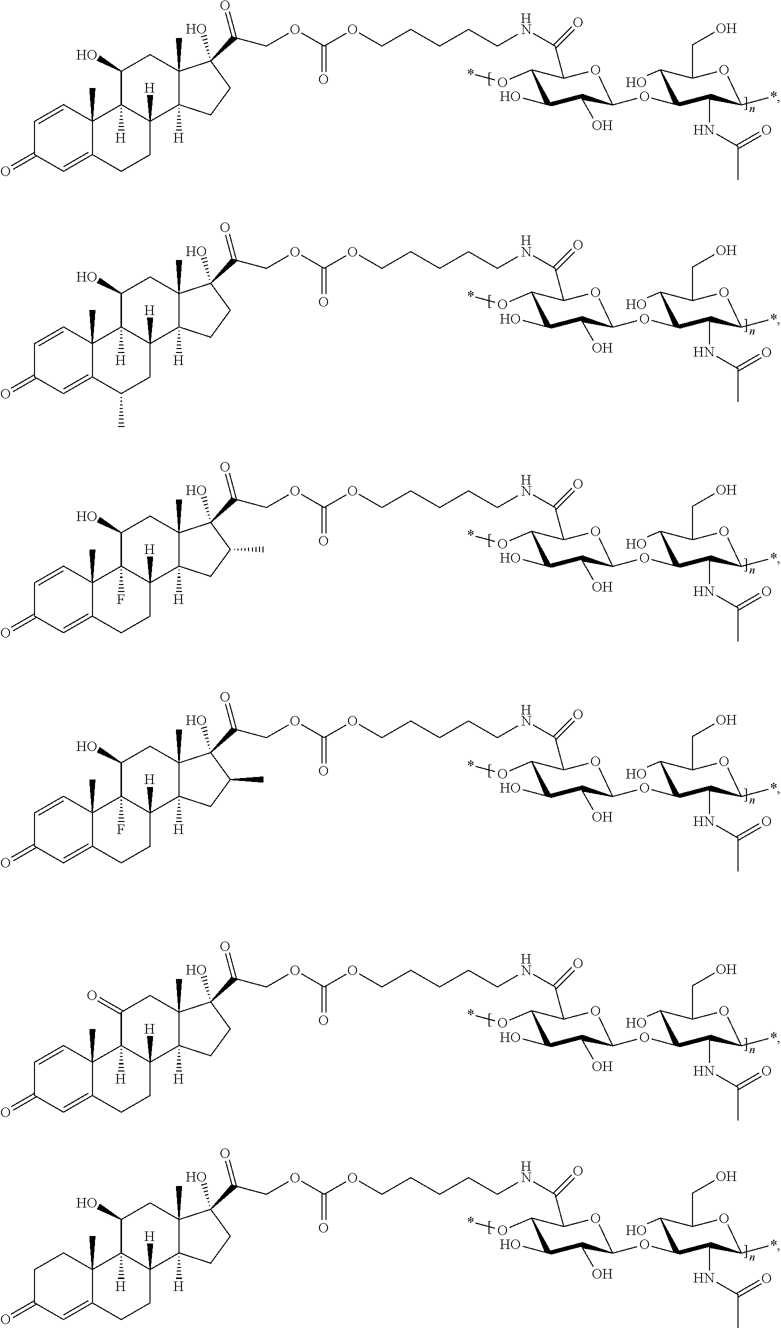

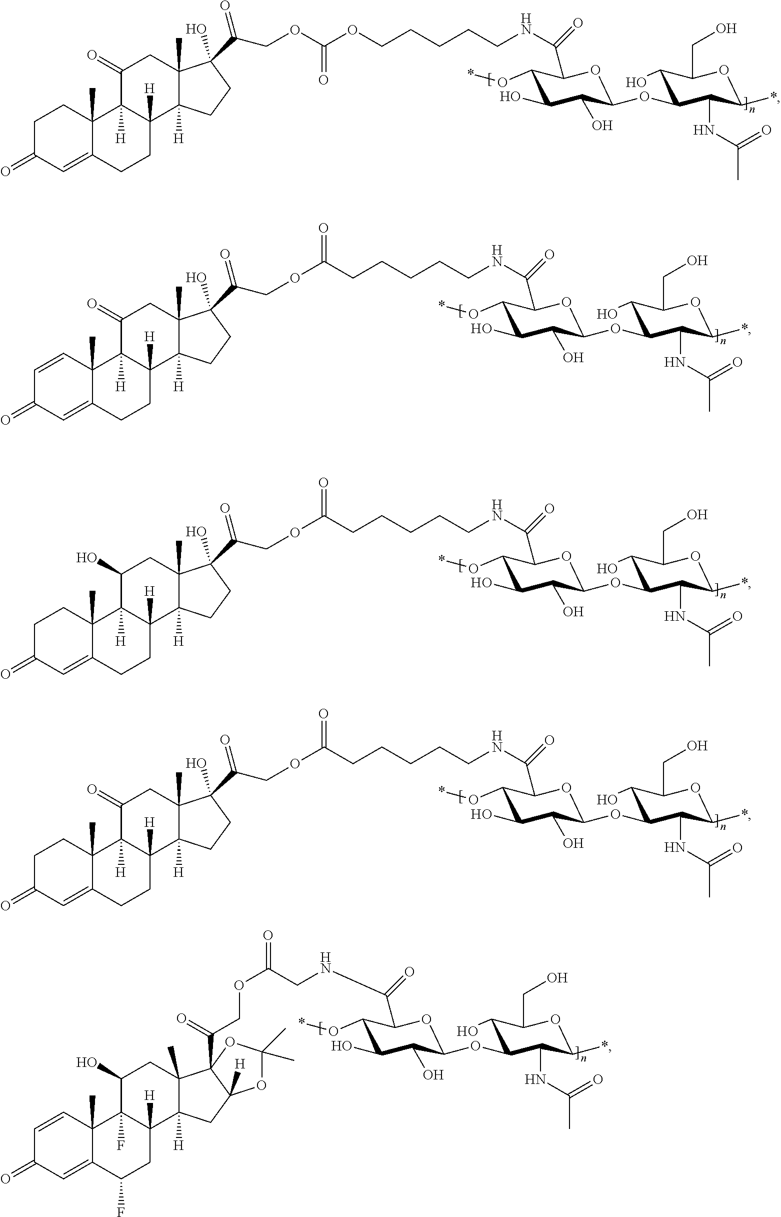

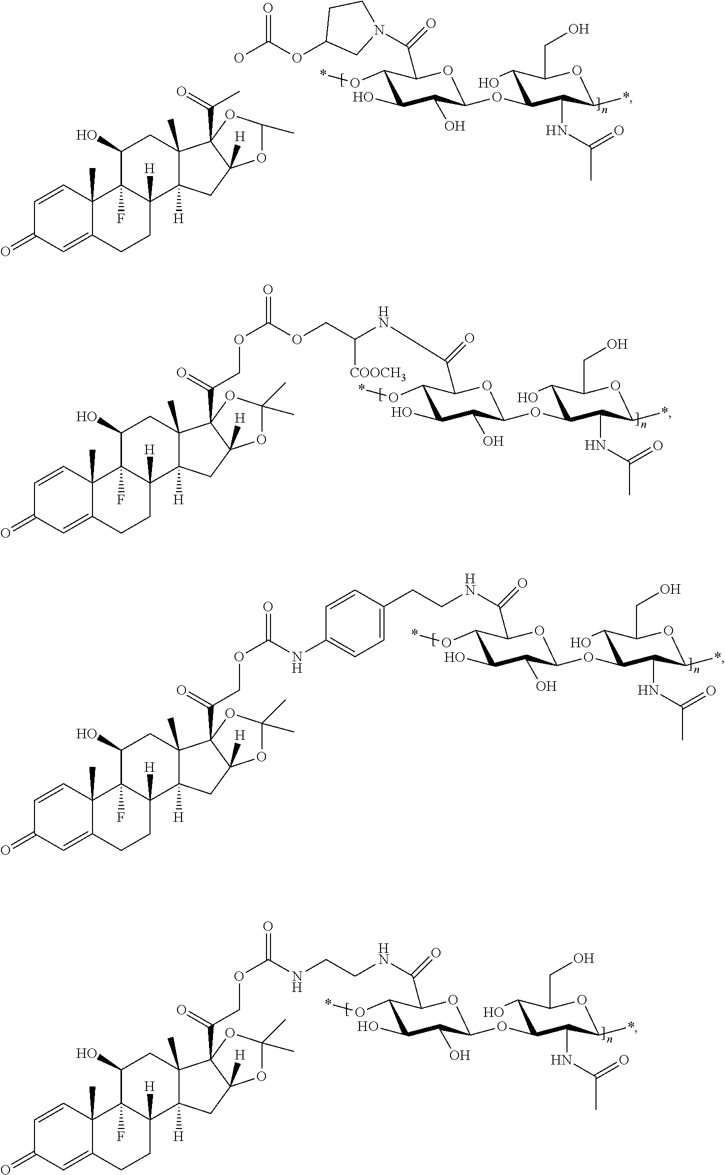

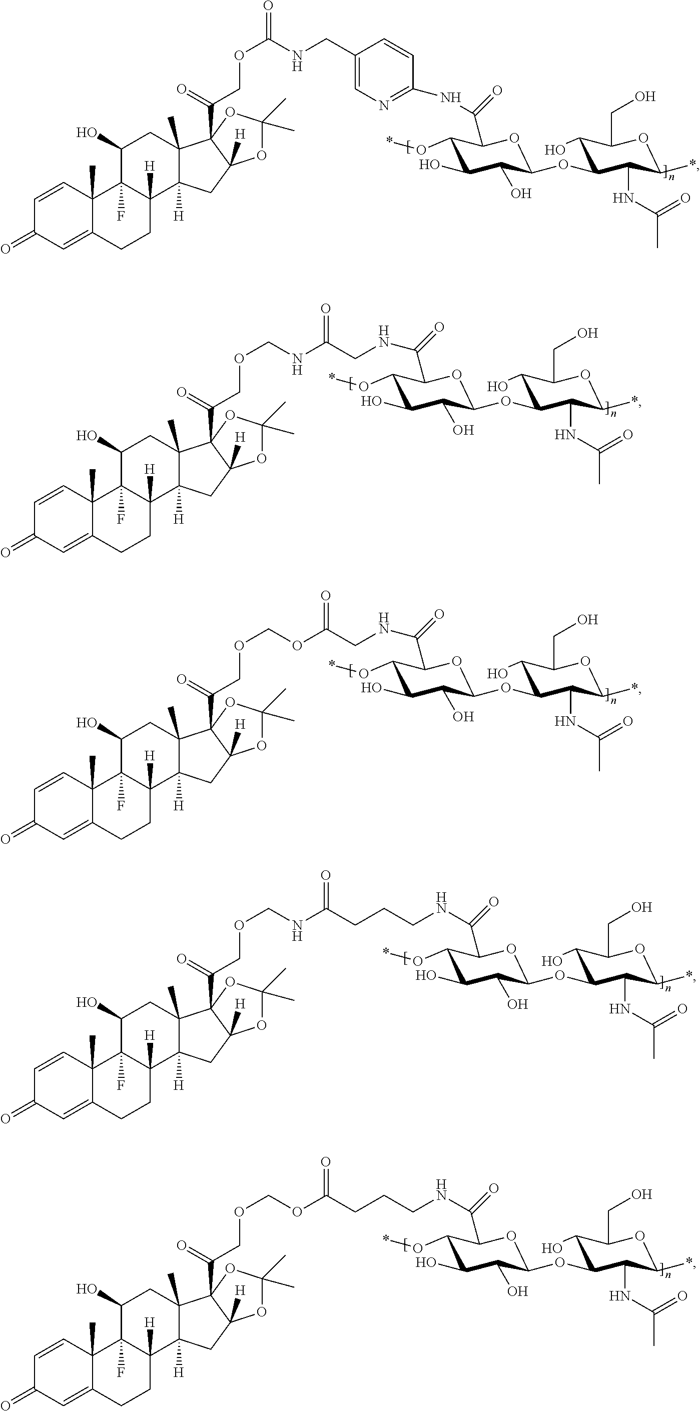

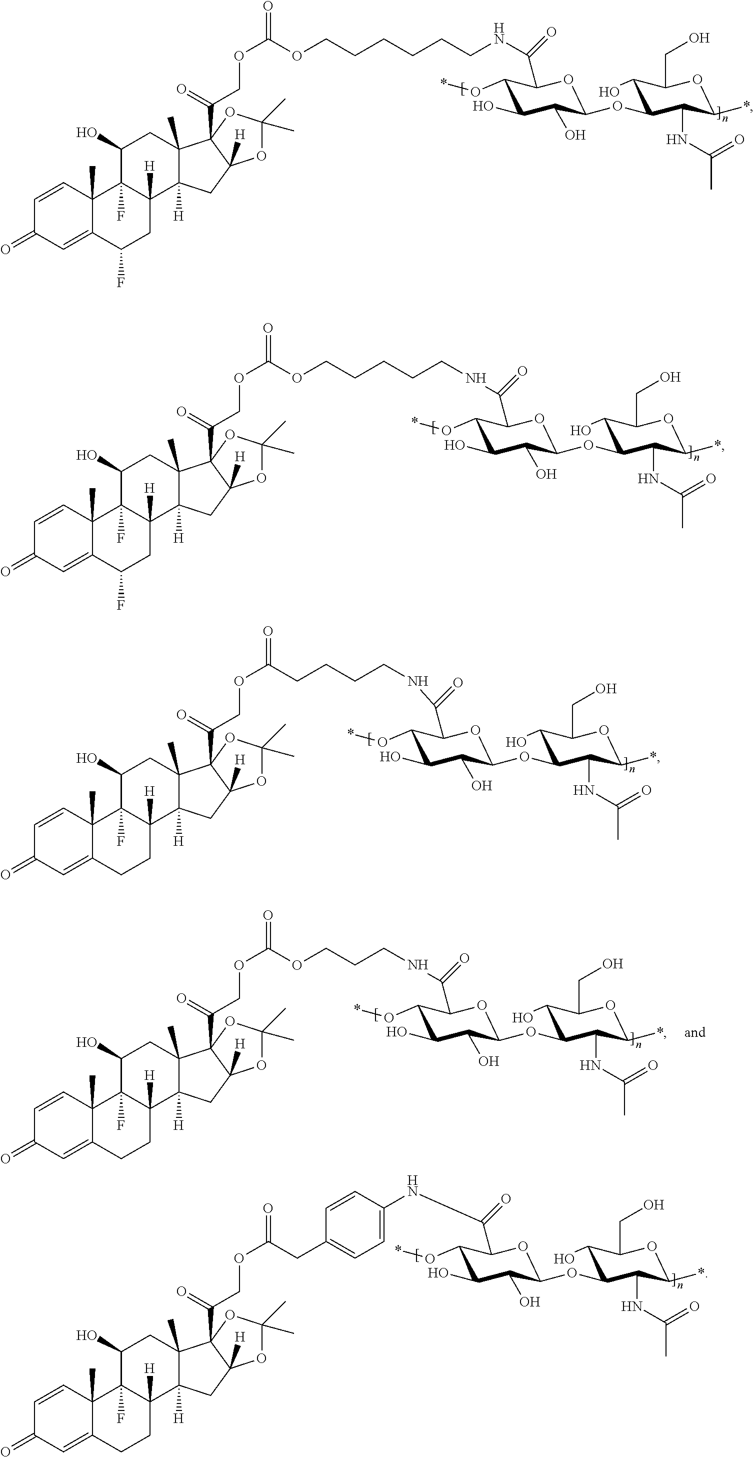

35. The drug delivery system of any one of claims 1-34, wherein the drug delivery system is locally administrated to a subject in need thereof.

36. The drug delivery system of claim 35, wherein the drug delivery system is locally administered to a subject in need thereof via injection.

37. The drug delivery system of claim 35, wherein the drug delivery system is locally administered to a subject in need thereof via oral dosage form.

38. The drug delivery system of claim 35, wherein the drug delivery system is locally administered to a subject in need thereof via inhalation.

39. The drug delivery system of claim 35, wherein the drug delivery system is locally administered to a subject in need thereof via implant.

40. The drug delivery system of claim 35, wherein the drug delivery system is locally administered to a subject in need thereof via topical application.

41. A pharmaceutical composition comprising the drug delivery system according to any one of claims 1-40 and a pharmaceutically acceptable excipient.

42. A method of treating a disorder in a subject in need thereof, the method comprising administering to the subject a therapeutic effective amount of the drug delivery system according to any one of claims 1-40 or the pharmaceutical composition according to claim 41.

43. The method according to claim 42, wherein the disorder is allergic diseases, autoimmune diseases, or inflammatory diseases.

44. The method according to claim 43, wherein the disorder is selected from the group consisting of allergic rhinitis, systemic lupus erythematosus, rheumatism, nephrotic syndrome, autoimmune hemolytic anemia, idiopathic thrombocytopenia purpura, Addison disease, neurodermatitis, cutaneous pruritus, tendinitis, inflammation, respiratory disease, osteoarthritis, Neovascular (Wet) Age-Related Macular Degeneration (AMD), Macular Edema Following Retinal Vein Occlusion (RVO), Diabetic Macular Edema (DME), Diabetic Retinopathy (DR), Myopic Choroidal Neovascularization (mCNV), Uveitic Macular Edema (UME), dermatitis, psoriasis, chronic obstructive pulmonary disease, and asthma.

45. The method according to claim 43, wherein the disorder is selected from the group consisting of tendinitis, osteoarthritis, Neovascular (Wet) Age-Related Macular Degeneration (AMD), Diabetic Macular Edema (DME), Uveitic Macular Edema (UME), dermatitis, psoriasis, chronic obstructive pulmonary disease, and asthma.
